(19) United States
(12) Patent Application Publication
(10) Pub. No.: US 2022/0056106 A1
(43) Pub. Date: Feb. 24, 2022

(54) METHODS, SYSTEMS, AND KITS FOR TREATING INFLAMMATORY DISEASE TARGETING IL18R1

(71) Applicant: Cedars-Sinai Medical Center, Los Angeles, CA (US)

(72) Inventors: Janine BILSBOROUGH, Adelaide (AU); Stephan R. TARGAN, Santa Monica, CA (US); Dermot P. MCGOVERN, Los Angeles, CA (US)

(21) Appl. No.: 17/396,581

(22) Filed: Aug. 6, 2021

Related U.S. Application Data
(63) Continuation of application No. PCT/US2020/017212, filed on Feb. 7, 2020.
(60) Provisional application No. 62/802,828, filed on Feb. 8, 2019.
(60) Provisional application No. 62/815,223, filed on Mar. 7, 2019.

(51) Int. Cl. C07K 14/715; C07K 14/47; C07K 16/28; A61P 37/02

(52) U.S. Cl. CPC C07K 14/7155; C07K 14/4702; A61K 38/00; A61P 37/02; C07K 16/2875

(57) ABSTRACT

Described herein are methods, systems, compositions, and kits useful for the diagnosis and/or treatment of a disease or condition in a subject. The present disclosure relates to methods and systems for identifying and stratifying patients suitable for treatment with an IL18R1 modulator, as described herein.

### CROSS-REFERENCE

This application is a continuation of International Application No. PCT/US2020/017212, filed Feb. 7, 2020, which claims the benefit of U.S. Provisional Application No. 62/802,828 filed Feb. 8, 2019 and U.S. Provisional Application No. 62/815,223 filed Mar. 7, 2019, which are incorporated by reference herein in their entirety.

### SEQUENCE LISTING

This application contains a Sequence Listing which has been submitted electronically in ASCII format and is hereby incorporated by reference in its entirety. Said ASCII copy, created on Aug. 6, 2021, is named 52388-751_301_SL.txt and is 19,985 bytes in size.

### SUMMARY

Inflammatory bowel diseases (IBD) is a heterogeneous group of chronic, relapsing inflammatory disorders of the gastrointestinal (GI) tract affecting more than 3 million adults in the United States, according to the most recent Centers for Disease and Prevention (CDC) survey. The two most common manifestations of IBD are Crohn's disease (CD) and ulcerative colitis (UC). Each of these forms of IBD has various subclinical phenotypes that manifest in certain IBD patients. For example, the chronic inflammation of the GI tract caused by CD and UC leads to the formation of scar tissue (fibrosis) and stenosis (fibrostenosis) in the intestinal wall in some IBD patients, that is largely unresponsive to current therapeutic interventions. For these patients endoscopic or surgical treatment is often the only treatment available.

IBD, including CD and UC, is characterized by an uncontrolled activity of the immune response within the intestinal mucosal, which depends on genetic susceptibility to developing the IBD, subclinical phenotypes of IBD, as well as to various stimuli related to IBD pathogenesis (e.g., intestinal microbiome). Genome Wide Association Studies (GWAS) have enabled scientists to identify genetic variants in certain IBD susceptibility gene loci useful for the selection of IBD patients for treatment with targeted therapeutic strategies, and identifying drugable targets in the development of novel therapies.

Few treatment options are available to patients that suffer from IBD. Current therapeutic regimens include one or more of anti-inflammatory medication (e.g., corticosteroids) and immunomodulatory therapy (e.g., anti-TNF therapy). However, nearly half of all patients treated with an anti-TNF therapy do not respond to the induction of the therapy, or experience a loss of response to the treatment after a period of time, during which, disease severity has progressed significantly. Therefore, there remains a significant need for targeted and effective treatment options that respond to the underlying immunopathogenesis of IBD.

Interleukin 18 (IL18) is a member of the IL-1 cytokine family, which consists of eleven members that play important roles in regulating inflammation, including IL-1 alpha, IL-1 beta, IL-1ra, IL-18, IL-33, IL-36Ra, IL-36 alpha, IL-36 beta, IL-36 gamma, IL-37, and IL-38. While most of these cytokines are biologically active as full-length molecules, activation and secretion of IL-1 beta and IL18 requires inflammasome/Caspase-1-dependent processing. IL18R1 specifically binds IL18, and is essential for IL18 mediated signal transduction. In normal tissues IL-18 can be found in whole blood and in epithelium of the lung and small intestine.

The IL18 receptor (IL-18R) is composed of two subunits, IL18 receptor alpha (IL-18Rα) and IL18Rβ (or IL-18Rap), both of which consist of three extracellular immunoglobulin-like domains and one intracellular Toll/IL-1 receptor (TIR) domain. Ligand binding triggers receptor heterodimerization and initiates downstream signaling events via the two TIR domains, which recruit MyD88 and signal through IL-1R-associated kinases (IRAK) to initiate p38 MAPK and NF mediated responses.

IL-18 expression is increased in mucosal biopsies of IBD patients compared with those of control patients, and increased in inflamed versus noninflamed intestinal tissues of IBD patients. IL18 in IBD may be found in intestinal epithelial cells, and lamina propria (LP) macrophages and dendritic cells in more severe disease state. High levels of IL18 expression in lymphoid follicles in the LP of CD patients has been found in close association with CD4+ T cells.

IL18 is critical in driving the pathologic breakdown of barrier integrity in a model of colitis. Deletion of IL18 or IL18R1 in intestinal epithelial cells conferred protection from colitis and mucosal damage in mice. In contrast, deletion of the IL18 negative regulator IL18 bp resulted in severe colitis associated with loss of mature goblet cells. Colitis and goblet cell loss were rescued in IL18 bp(−/−); IL18r(A/EC) mice, demonstrating that colitis severity is controlled at the level of IL18 signaling in intestinal epithelial cells. IL18 inhibited goblet cell maturation by regulating the transcriptional program instructing goblet cell development. These results inform on the mechanism of goblet cell dysfunction underlying the pathology of ulcerative colitis.

IL18 has a pathogenic role in the intestine in murine models of CD4+ T cell mediated colitis. In addition, in mice lacking key inflammasome components that regulate the processing and secretion of IL-18, IL-18 may provide a tissue-protective role following injury to the intestinal epithelium, indicating that the role of IL-18 in intestinal immune regulation may be variable.

IL18R1 expression in vivo is enhanced on both effector and regulatory CD4+ T cells in the intestinal lamina propria, with Th17 cells exhibiting particularly high levels. During steady state, intestinal epithelial cells (IEC) constitutively secrete IL18 that acts directly on IL18R1-expressing CD4+ T cells to limit colonic Th17 cell differentiation, in part by antagonizing IL1R1-signalling. In addition, IL18R1 signaling has been shown to be critical for Foxp3+Treg cell mediated control of intestinal inflammation, where it promotes expression of Treg effector molecules. Thus, IL18R1 may be a promising target for the treatment of disease or conditions associated with inflammation, and cellular immunity, such as inflammatory bowel disease (e.g., IBD, CD, UC).

Aspects disclosed herein are practical applications of patient selection criteria useful for the identification and selection of subjects suitable for treatment with a particular therapeutic agent to treat one or more inflammatory diseases or disorders described herein (e.g., inflammatory bowel diseases), or subclinical phenotypes thereof. In some instances, the patient selection criteria are useful for the identification and selection of subjects not suitable for a treatment with a standard therapy (e.g., anti-TNF therapy, corticosteroid, thiopurine). In some instances, the patient selection criteria comprise polymorphisms or aberrations of a gene or gene expression product. In some instances the patient selection criteria comprise single nucleotide variants (SNVs), single nucleotide polymorphisms (SNPs), insertion or deletions (indels), and the like. In some instances, expression of a gene expression product (e.g., a biomarker), alone, or in combination with the polymorphisms are useful as patient selection criteria. In some instances, the polymorphism or aberration is located at a gene or genetic locus encoding at least a part of the target of the therapeutic agent (e.g., IL18R1). In some instances, that polymorphism or aberration is associated with an increase or a decrease in the expression of the target gene expression product. For example, disclosed herein is overlapping eQTL and subclinical phenotype association data suggesting the minor allele for eQTL in UC rectum is associated with upregulation of gene expression. The minor allele is also associated with risk for colonic disease and time to first surgery but is protective for smoking history. This data is consistent with the idea that increased IL18R1 expression is associated with disease in UC.

Aspects disclosed herein provide methods of treating or preventing a disease or condition in a subject, the method comprising administering a modulator of Interleukin 18 Receptor 1 (IL18R1) activity or expression to the subject, provided a genotype is detected in a sample obtained the subject. In some embodiments, the genotype is detected with an assay comprising polymerase chain reaction (PCR), quantitative reverse-transcription PCR (qPCR), automated sequencing, genotype array, or a combination thereof. In some embodiments, the modulator of IL18R1 activity or expression comprises an agonist or a partial agonist of IL18R1. In some embodiments, the modulator of IL18R1 activity or expression comprises an antagonist or partial antagonist of IL18R1. In some embodiments, the agonist or partial agonist comprises an antibody or antigen-binding fragment, peptide, small molecule. In some embodiments, the modulator of IL18R1 activity or expression comprises an inverse agonist. In some embodiments, the modulator of IL18R1 activity or expression comprises a positive allosteric modulator (PAM). In some embodiments, the modulator of IL18R1 activity or expression comprises a negative allosteric modulator (NAM). In some embodiments, the modulator of IL18R1 activity or expression comprises a small molecule that binds to IL18R1 or IL18, or both. In some embodiments, the modulator of IL18R1 activity or expression comprises an antibody or antigen binding fragment that binds to IL18R1 or IL18, or both. In some embodiments, the modulator of IL18R1 activity or expression comprises recombinant IL18R1 peptide, or a recombinant IL18 peptide. In some embodiments, the modulator of IL18R1 activity or expression comprises a recombinant peptide comprising an amino acid sequence that is about 70%, 75%, 80%, 85%, 90%, 91%, 92%, 93%, 94%, 95%, 96%, 97%, 98%, 99%, or 100% homologous to SEQ ID NO: 8 or SEQ ID NO: 9. In some embodiments, the modulator of IL18R1 activity or expression comprises a recombinant peptide comprising an amino acid sequence that is about 70%, 75%, 80%, 85%, 90%, 91%, 92%, 93%, 94%, 95%, 96%, 97%, 98%, 99%, or 100% homologous to SEQ ID NO: 8 or SEQ ID NO: 9, and wherein the amino acid sequence is truncated at the N-terminal and/or C-terminal ends of the peptide. In some embodiments, the modulator of IL18R1 activity or expression comprises a fusion, a conjugate, or both. In some embodiments, the modulator of IL18R1 activity or expression comprises an agonist or an antagonist of IL18 signaling. In some embodiments, the modulator of IL18R1 activity or expression comprises an agonist or an antagonist of IL18-IL18R1 binding. In some embodiments, the genotype is homozygous or heterozygous. In some embodiments, the disease or condition comprises and inflammatory, fibrostenotic, and/or fibrotic disease or condition. In some embodiments, the inflammatory, fibrostenotic, and/or fibrotic disease or condition comprises inflammatory bowel disease (IBD), Crohn's disease (CD), perianal CD, ulcerative colitis (UC), multiple sclerosis (MS), rheumatoid arthritis (RA), primary sclerosing cholangitis (PSC), Pancolitis, primary billary cihrosis, asthma, Proctitis, Iritis, intestinal fibrosis, pulmonary fibrosis, or intestinal fibrostenosis. In some embodiments, the sample comprises whole blood, plasma, serum, or biopsy tissue. In some embodiments, the subject is mammal. In some embodiments, the subject is human. In some embodiments, the subject is non-responsive to an induction of anti-Tumor Necrosis Factor (TNF) therapy, or lost response to the anti-TNF therapy after a period of time during treatment. In some embodiments, the inflammatory, fibrostenotic, and/or fibrotic disease is refractory. In some embodiments, the genotype comprises one or more single nucleotide polymorphisms (SNPs) or indels at rs1921622, rs2287037, rs1974675, rs2041739, rs76362690, rs2287037, or rs80256362, a SNP in linkage disequilibrium (LD) therewith, or any combination thereof. In some embodiments, the SNP at rs1921622 comprises an “A” or a “G” on a forward DNA strand encoding the SNP. In some embodiments, the SNP at rs2287037 comprises an “A” or a “G” on a reverse DNA strand encoding the SNP. The method of claim 30, wherein the SNP at rs10213846 comprises a “G” or a “T” allele. In some embodiments, the SNP at rs1974675 comprises a “C” or a “T” on a reverse DNA strand encoding the SNP. In some embodiments, the SNP at rs2041739 comprises an “A” or a “G” on a reverse DNA strand encoding the SNP. In some embodiments, the SNP at rs76362690 comprises an “A” or a “G” on a forward DNA strand encoding the SNP. In some embodiments, the SNP at rs2287037 comprises an “A” or a “G” on a reverse DNA strand encoding the SNP. In some embodiments, the SNP at rs80256362 comprises an “A” or a “G” on a forward DNA strand encoding the SNP. In some embodiments, the Indel at rs1921622 is within SEQ ID NO: 1. In some embodiments, the SNP at rs2287037 is within SEQ ID NO: 2. In some embodiments, the SNP at rs1974675 is within SEQ ID NO: 3. In some embodiments, the SNP at rs2041739 is within s SEQ ID NO: 4. In some embodiments, the SNP at rs76362690 is within SEQ ID NO: 5. In some embodiments, the SNP at rs2287037 is within SEQ ID NO: 6. In some embodiments, the SNP at rs80256362 is within SEQ ID NO: 7. In some embodiments, LD is defined by an r² value of at least 0.80, 0.85, 0.90, 0.95, or 1.0. In some embodiments, the genotype is associated with a risk that a subject has, or will develop, inflammatory bowel disease (IBD), Crohn's disease (CD), or ulcerative colitis (UC), as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰. In some embodiments, the genotype is associated with a risk that the subject has, or will develop, a subclinical phenotype of the disease or condition as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰. In some embodiments, the subclinical phenotype comprises stricturing, penetrating, or stricturing and penetrating, disease phenotypes. In some embodiments, the genotype comprises one or more SNPs in linkage disequilibrium with rs1921622 as determined by an r² value of at least about 0.80, about 0.85, about 0.90, about 0.95, or about 1.0.

Aspects disclosed herein provide methods of reducing or ablating activity or expression of Interleukin 18 Receptor 1(IL18R1) in a subject, the method comprising administering a modulator of IL18R1 to the subject, provided a genotype is detected in a sample obtained from the subject. In some embodiments, the genotype is detected with an assay comprising polymerase chain reaction (PCR), quantitative reverse-transcription PCR (qPCR), automated sequencing, genotype array, or a combination thereof. In some embodiments, the modulator of IL18R1 activity or expression comprises an agonist or a partial agonist of IL18R1. In some embodiments, the modulator of IL18R1 activity or expression comprises an antagonist or partial antagonist of IL18R1. In some embodiments, the agonist or partial agonist comprises an antibody or antigen-binding fragment, peptide, small molecule. In some embodiments, the modulator of IL18R1 activity or expression comprises an inverse agonist. In some embodiments, the modulator of IL18R1 activity or expression comprises a positive allosteric modulator (PAM). In some embodiments, the modulator of IL18R1 activity or expression comprises a negative allosteric modulator (NAM). In some embodiments, the modulator of IL18R1 activity or expression comprises a small molecule that binds to IL18R1 or IL18, or both. In some embodiments, the modulator of IL18R1 activity or expression comprises an antibody or antigen binding fragment that binds to IL18R1 or IL18, or both. In some embodiments, the modulator of IL18R1 activity or expression comprises recombinant IL18R1 peptide, or a recombinant IL18 peptide. In some embodiments, the modulator of IL18R1 activity or expression comprises a recombinant peptide comprising an amino acid sequence that is about 70%, 75%, 80%, 85%, 90%, 91%, 92%, 93%, 94%, 95%, 96%, 97%, 98%, 99%, or 100% homologous to SEQ ID NO: 8 or SEQ ID NO: 9. In some embodiments, the modulator of IL18R1 activity or expression comprises a recombinant peptide comprising an amino acid sequence that is about 70%, 75%, 80%, 85%, 90%, 91%, 92%, 93%, 94%, 95%, 96%, 97%, 98%, 99%, or 100% homologous to SEQ ID NO: 8 or SEQ ID NO: 9, and wherein the amino acid sequence is truncated at the N-terminal and/or C-terminal ends of the peptide. In some embodiments, the modulator of IL18R1 activity or expression comprises a fusion, a conjugate, or both. In some embodiments, the modulator of IL18R1 activity or expression comprises an agonist or an antagonist of IL18 signaling. In some embodiments, the modulator of IL18R1 activity or expression comprises an agonist or an antagonist of IL18-IL18R1 binding. In some embodiments, the genotype is homozygous or heterozygous. In some embodiments, the disease or condition comprises and inflammatory, fibrostenotic, and/or fibrotic disease or condition. In some embodiments, the inflammatory, fibrostenotic, and/or fibrotic disease or condition comprises inflammatory bowel disease (IBD), Crohn's disease (CD), perianal CD, ulcerative colitis (UC), multiple sclerosis (MS), rheumatoid arthritis (RA), primary sclerosing cholangitis (PSC), Pancolitis, primary billary cihrosis, asthma, Proctitis, Iritis, intestinal fibrosis, pulmonary fibrosis, or intestinal fibrostenosis. In some embodiments, the sample comprises whole blood, plasma, serum, or biopsy tissue. In some embodiments, the subject is mammal. In some embodiments, the subject is human. In some embodiments, the subject is non-responsive to an induction of anti-Tumor Necrosis Factor (TNF) therapy, or lost response to the anti-TNF therapy after a period of time during treatment. In some embodiments, the inflammatory, fibrostenotic, and/or fibrotic disease is refractory. In some embodiments, the genotype comprises one or more single nucleotide polymorphisms (SNPs) or indels at rs1921622, rs2287037, rs1974675, rs2041739, rs76362690, rs2287037, or rs80256362, a SNP in linkage disequilibrium (LD) therewith, or any combination thereof. In some embodiments, the SNP at rs1921622 comprises an “A” or a “G” on a forward DNA strand encoding the SNP. In some embodiments, the SNP at rs2287037 comprises an “A” or a “G” on a reverse DNA strand encoding the SNP. The method of claim 30, wherein the SNP at rs10213846 comprises a “G” or a “T” allele. In some embodiments, the SNP at rs1974675 comprises a “C” or a “T” on a reverse DNA strand encoding the SNP. In some embodiments, the SNP at rs2041739 comprises an “A” or a “G” on a reverse DNA strand encoding the SNP. In some embodiments, the SNP at rs76362690 comprises an “A” or a “G” on a forward DNA strand encoding the SNP. In some embodiments, the SNP at rs2287037 comprises an “A” or a “G” on a reverse DNA strand encoding the SNP. In some embodiments, the SNP at rs80256362 comprises an “A” or a “G” on a forward DNA strand encoding the SNP. In some embodiments, the Indel at rs1921622 is within SEQ ID NO: 1. In some embodiments, the SNP at rs2287037 is within SEQ ID NO: 2. In some embodiments, the SNP at rs1974675 is within SEQ ID NO: 3. In some embodiments, the SNP at rs2041739 is within s SEQ ID NO: 4. In some embodiments, the SNP at rs76362690 is within SEQ ID NO: 5. In some embodiments, the SNP at rs2287037 is within SEQ ID NO: 6. In some embodiments, the SNP at rs80256362 is within SEQ ID NO: 7. In some embodiments, LD is defined by an r² value of at least 0.80, 0.85, 0.90, 0.95, or 1.0. In some embodiments, the genotype is associated with a risk that a subject has, or will develop, inflammatory bowel disease (IBD), Crohn's disease (CD), or ulcerative colitis (UC), as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰. In some embodiments, the genotype is associated with a risk that the subject has, or will develop, a subclinical phenotype of the disease or condition as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰. In some embodiments, the subclinical phenotype comprises stricturing, penetrating, or stricturing and penetrating, disease phenotypes. In some embodiments, the genotype comprises one or more SNPs in linkage disequilibrium with rs1921622 as determined by an r² value of at least about 0.80, about 0.85, about 0.90, about 0.95, or about 1.0.

Aspects disclosed herein provide methods of treating or preventing a disease or condition in a subject, the method comprising: obtaining a sample from a subject; detecting a presence or an absence of a genotype in the sample obtained from the subject; and administering to the subject a modulator of Interleukin 18 Receptor 1(IL18R1) activity or expression to the subject, provided the presence of the genotype is detected in the sample obtained from the subject. In some embodiments, the genotype is detected with an assay comprising polymerase chain reaction (PCR), quantitative reverse-transcription PCR (qPCR), automated sequencing, genotype array, or a combination thereof. In some embodiments, the modulator of IL18R1 activity or expression comprises an agonist or a partial agonist of IL18R1. In some embodiments, the modulator of IL18R1 activity or expression comprises an antagonist or partial antagonist of IL18R1. In some embodiments, the agonist or partial agonist comprises an antibody or antigen-binding fragment, peptide, small molecule. In some embodiments, the modulator of IL18R1 activity or expression comprises an inverse agonist. In some embodiments, the modulator of IL18R1 activity or expression comprises a positive allosteric modulator (PAM). In some embodiments, the modulator of IL18R1 activity or expression comprises a negative allosteric modulator (NAM). In some embodiments, the modulator of IL18R1 activity or expression comprises a small molecule that binds to IL18R1 or IL18, or both. In some embodiments, the modulator of IL18R1 activity or expression comprises an antibody or antigen binding fragment that binds to IL18R1 or IL18, or both. In some embodiments, the modulator of IL18R1 activity or expression comprises recombinant IL18R1 peptide, or a recombinant IL18 peptide. In some embodiments, the modulator of IL18R1 activity or expression comprises a recombinant peptide comprising an amino acid sequence that is about 70%, 75%, 80%, 85%, 90%, 91%, 92%, 93%, 94%, 95%, 96%, 97%, 98%, 99%, or 100% homologous to SEQ ID NO: 8 or SEQ ID NO: 9. In some embodiments, the modulator of IL18R1 activity or expression comprises a recombinant peptide comprising an amino acid sequence that is about 70%, 75%, 80%, 85%, 90%, 91%, 92%, 93%, 94%, 95%, 96%, 97%, 98%, 99%, or 100% homologous to SEQ ID NO: 8 or SEQ ID NO: 9, and wherein the amino acid sequence is truncated at the N-terminal and/or C-terminal ends of the peptide. In some embodiments, the modulator of IL18R1 activity or expression comprises a fusion, a conjugate, or both. In some embodiments, the modulator of IL18R1 activity or expression comprises an agonist or an antagonist of IL18 signaling. In some embodiments, the modulator of IL18R1 activity or expression comprises an agonist or an antagonist of IL18-IL18R1 binding. In some embodiments, the genotype is homozygous or heterozygous. In some embodiments, the disease or condition comprises and inflammatory, fibrostenotic, and/or fibrotic disease or condition. In some embodiments, the inflammatory, fibrostenotic, and/or fibrotic disease or condition comprises inflammatory bowel disease (IBD), Crohn's disease (CD), perianal CD, ulcerative colitis (UC), multiple sclerosis (MS), rheumatoid arthritis (RA), primary sclerosing cholangitis (PSC), Pancolitis, primary billary cihrosis, asthma, Proctitis, Iritis, intestinal fibrosis, pulmonary fibrosis, or intestinal fibrostenosis. In some embodiments, the sample comprises whole blood, plasma, serum, or biopsy tissue. In some embodiments, the subject is mammal. In some embodiments, the subject is human. In some embodiments, the subject is non-responsive to an induction of anti-Tumor Necrosis Factor (TNF) therapy, or lost response to the anti-TNF therapy after a period of time during treatment. In some embodiments, the inflammatory, fibrostenotic, and/or fibrotic disease is refractory. In some embodiments, the genotype comprises one or more single nucleotide polymorphisms (SNPs) or indels at rs1921622, rs2287037, rs1974675, rs2041739, rs76362690, rs2287037, or rs80256362, a SNP in linkage disequilibrium (LD) therewith, or any combination thereof. In some embodiments, the SNP at rs1921622 comprises an “A” or a “G” on a forward DNA strand encoding the SNP. In some embodiments, the SNP at rs2287037 comprises an “A” or a “G” on a reverse DNA strand encoding the SNP. The method of claim 30, wherein the SNP at rs10213846 comprises a “G” or a “T” allele. In some embodiments, the SNP at rs1974675 comprises a “C” or a “T” on a reverse DNA strand encoding the SNP. In some embodiments, the SNP at rs2041739 comprises an “A” or a “G” on a reverse DNA strand encoding the SNP. In some embodiments, the SNP at rs76362690 comprises an “A” or a “G” on a forward DNA strand encoding the SNP. In some embodiments, the SNP at rs2287037 comprises an “A” or a “G” on a reverse DNA strand encoding the SNP. In some embodiments, the SNP at rs80256362 comprises an “A” or a “G” on a forward DNA strand encoding the SNP. In some embodiments, the Indel at rs1921622 is within SEQ ID NO: 1. In some embodiments, the SNP at rs2287037 is within SEQ ID NO: 2. In some embodiments, the SNP at rs1974675 is within SEQ ID NO: 3. In some embodiments, the SNP at rs2041739 is within s SEQ ID NO: 4. In some embodiments, the SNP at rs76362690 is within SEQ ID NO: 5. In some embodiments, the SNP at rs2287037 is within SEQ ID NO: 6. In some embodiments, the SNP at rs80256362 is within SEQ ID NO: 7. In some embodiments, LD is defined by an r² value of at least 0.80, 0.85, 0.90, 0.95, or 1.0. In some embodiments, the genotype is associated with a risk that a subject has, or will develop, inflammatory bowel disease (IBD), Crohn's disease (CD), or ulcerative colitis (UC), as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰. In some embodiments, the genotype is associated with a risk that the subject has, or will develop, a subclinical phenotype of the disease or condition as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰. In some embodiments, the subclinical phenotype comprises stricturing, penetrating, or stricturing and penetrating, disease phenotypes. In some embodiments, the genotype comprises one or more SNPs in linkage disequilibrium with rs1921622 as determined by an r² value of at least about 0.80, about 0.85, about 0.90, about 0.95, or about 1.0.

Aspects disclosed herein provide methods of reducing, ablating, increasing, or activating, an activity or expression of Interleukin 18 Receptor 1 (IL18R1) in a subject, the method comprising: obtaining a sample from a subject; detecting a presence or an absence of a genotype in the sample obtained from the subject; and administering to the subject a modulator of IL18R1 activity or expression to the subject, provided the presence of the genotype is detected in the sample obtained from the subject. In some embodiments, the genotype is detected with an assay comprising polymerase chain reaction (PCR), quantitative reverse-transcription PCR (qPCR), automated sequencing, genotype array, or a combination thereof. In some embodiments, the modulator of IL18R1 activity or expression comprises an agonist or a partial agonist of IL18R1. In some embodiments, the modulator of IL18R1 activity or expression comprises an antagonist or partial antagonist of IL18R1. In some embodiments, the agonist or partial agonist comprises an antibody or antigen-binding fragment, peptide, small molecule. In some embodiments, the modulator of IL18R1 activity or expression comprises an inverse agonist. In some embodiments, the modulator of IL18R1 activity or expression comprises a positive allosteric modulator (PAM). In some embodiments, the modulator of IL18R1 activity or expression comprises a negative allosteric modulator (NAM). In some embodiments, the modulator of IL18R1 activity or expression comprises a small molecule that binds to IL18R1 or IL18, or both. In some embodiments, the modulator of IL18R1 activity or expression comprises an antibody or antigen binding fragment that binds to IL18R1 or IL18, or both. In some embodiments, the modulator of IL18R1 activity or expression comprises recombinant IL18R1 peptide, or a recombinant IL18 peptide. In some embodiments, the modulator of IL18R1 activity or expression comprises a recombinant peptide comprising an amino acid sequence that is about 70%, 75%, 80%, 85%, 90%, 91%, 92%, 93%, 94%, 95%, 96%, 97%, 98%, 99%, or 100% homologous to SEQ ID NO: 8 or SEQ ID NO: 9. In some embodiments, the modulator of IL18R1 activity or expression comprises a recombinant peptide comprising an amino acid sequence that is about 70%, 75%, 80%, 85%, 90%, 91%, 92%, 93%, 94%, 95%, 96%, 97%, 98%, 99%, or 100% homologous to SEQ ID NO: 8 or SEQ ID NO: 9, and wherein the amino acid sequence is truncated at the N-terminal and/or C-terminal ends of the peptide. In some embodiments, the modulator of IL18R1 activity or expression comprises a fusion, a conjugate, or both. In some embodiments, the modulator of IL18R1 activity or expression comprises an agonist or an antagonist of IL18 signaling. In some embodiments, the modulator of IL18R1 activity or expression comprises an agonist or an antagonist of IL18-IL18R1 binding. In some embodiments, the genotype is homozygous or heterozygous. In some embodiments, the disease or condition comprises and inflammatory, fibrostenotic, and/or fibrotic disease or condition. In some embodiments, the inflammatory, fibrostenotic, and/or fibrotic disease or condition comprises inflammatory bowel disease (IBD), Crohn's disease (CD), perianal CD, ulcerative colitis (UC), multiple sclerosis (MS), rheumatoid arthritis (RA), primary sclerosing cholangitis (PSC), Pancolitis, primary billary cihrosis, asthma, Proctitis, Iritis, intestinal fibrosis, pulmonary fibrosis, or intestinal fibrostenosis. In some embodiments, the sample comprises whole blood, plasma, serum, or biopsy tissue. In some embodiments, the subject is mammal. In some embodiments, the subject is human. In some embodiments, the subject is non-responsive to an induction of anti-Tumor Necrosis Factor (TNF) therapy, or lost response to the anti-TNF therapy after a period of time during treatment. In some embodiments, the inflammatory, fibrostenotic, and/or fibrotic disease is refractory. In some embodiments, the genotype comprises one or more single nucleotide polymorphisms (SNPs) or indels at rs1921622, rs2287037, rs1974675, rs2041739, rs76362690, rs2287037, or rs80256362, a SNP in linkage disequilibrium (LD) therewith, or any combination thereof. In some embodiments, the SNP at rs1921622 comprises an “A” or a “G” on a forward DNA strand encoding the SNP. In some embodiments, the SNP at rs2287037 comprises an “A” or a “G” on a reverse DNA strand encoding the SNP. The method of claim 30, wherein the SNP at rs10213846 comprises a “G” or a “T” allele. In some embodiments, the SNP at rs1974675 comprises a “C” or a “T” on a reverse DNA strand encoding the SNP. In some embodiments, the SNP at rs2041739 comprises an “A” or a “G” on a reverse DNA strand encoding the SNP. In some embodiments, the SNP at rs76362690 comprises an “A” or a “G” on a forward DNA strand encoding the SNP. In some embodiments, the SNP at rs2287037 comprises an “A” or a “G” on a reverse DNA strand encoding the SNP. In some embodiments, the SNP at rs80256362 comprises an “A” or a “G” on a forward DNA strand encoding the SNP. In some embodiments, the Indel at rs1921622 is within SEQ ID NO: 1. In some embodiments, the SNP at rs2287037 is within SEQ ID NO: 2. In some embodiments, the SNP at rs1974675 is within SEQ ID NO: 3. In some embodiments, the SNP at rs2041739 is within s SEQ ID NO: 4. In some embodiments, the SNP at rs76362690 is within SEQ ID NO: 5. In some embodiments, the SNP at rs2287037 is within SEQ ID NO: 6. In some embodiments, the SNP at rs80256362 is within SEQ ID NO: 7. In some embodiments, LD is defined by an r² value of at least 0.80, 0.85, 0.90, 0.95, or 1.0. In some embodiments, the genotype is associated with a risk that a subject has, or will develop, inflammatory bowel disease (IBD), Crohn's disease (CD), or ulcerative colitis (UC), as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰. In some embodiments, the genotype is associated with a risk that the subject has, or will develop, a subclinical phenotype of the disease or condition as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰. In some embodiments, the subclinical phenotype comprises stricturing, penetrating, or stricturing and penetrating, disease phenotypes. In some embodiments, the genotype comprises one or more SNPs in linkage disequilibrium with rs1921622 as determined by an r² value of at least about 0.80, about 0.85, about 0.90, about 0.95, or about 1.0.

Aspects disclosed herein provide methods of diagnosing a disease or condition in a subject, the method comprising: obtaining a sample from a subject; detecting a presence or an absence of a genotype in the sample obtained from the subject; and diagnosing the disease or condition in the subject, provided the presence of the genotype is detected in the sample obtained from the subject. In some embodiments, the genotype is detected with an assay comprising polymerase chain reaction (PCR), quantitative reverse-transcription PCR (qPCR), automated sequencing, genotype array, or a combination thereof. In some embodiments, wherein the methods further comprise administering to the subject a modulator or IL18R1 activity or expression. In some embodiments, the modulator of IL18R1 activity or expression comprises an agonist or a partial agonist of IL18R1. In some embodiments, the modulator of IL18R1 activity or expression comprises an antagonist or partial antagonist of IL18R1. In some embodiments, the agonist or partial agonist comprises an antibody or antigen-binding fragment, peptide, small molecule. In some embodiments, the modulator of IL18R1 activity or expression comprises an inverse agonist. In some embodiments, the modulator of IL18R1 activity or expression comprises a positive allosteric modulator (PAM). In some embodiments, the modulator of IL18R1 activity or expression comprises a negative allosteric modulator (NAM). In some embodiments, the modulator of IL18R1 activity or expression comprises a small molecule that binds to IL18R1 or IL18, or both. In some embodiments, the modulator of IL18R1 activity or expression comprises an antibody or antigen binding fragment that binds to IL18R1 or IL18, or both. In some embodiments, the modulator of IL18R1 activity or expression comprises recombinant IL18R1 peptide, or a recombinant IL18 peptide. In some embodiments, the modulator of IL18R1 activity or expression comprises a recombinant peptide comprising an amino acid sequence that is about 70%, 75%, 80%, 85%, 90%, 91%, 92%, 93%, 94%, 95%, 96%, 97%, 98%, 99%, or 100% homologous to SEQ ID NO: 8 or SEQ ID NO: 9. In some embodiments, the modulator of IL18R1 activity or expression comprises a recombinant peptide comprising an amino acid sequence that is about 70%, 75%, 80%, 85%, 90%, 91%, 92%, 93%, 94%, 95%, 96%, 97%, 98%, 99%, or 100% homologous to SEQ ID NO: 8 or SEQ ID NO: 9, and wherein the amino acid sequence is truncated at the N-terminal and/or C-terminal ends of the peptide. In some embodiments, the modulator of IL18R1 activity or expression comprises a fusion, a conjugate, or both. In some embodiments, the modulator of IL18R1 activity or expression comprises an agonist or an antagonist of IL18 signaling. In some embodiments, the modulator of IL18R1 activity or expression comprises an agonist or an antagonist of IL18-IL18R1 binding. In some embodiments, the genotype is homozygous or heterozygous. In some embodiments, the disease or condition comprises and inflammatory, fibrostenotic, and/or fibrotic disease or condition. In some embodiments, the inflammatory, fibrostenotic, and/or fibrotic disease or condition comprises inflammatory bowel disease (IBD), Crohn's disease (CD), perianal CD, ulcerative colitis (UC), multiple sclerosis (MS), rheumatoid arthritis (RA), primary sclerosing cholangitis (PSC), Pancolitis, primary billary cihrosis, asthma, Proctitis, Iritis, intestinal fibrosis, pulmonary fibrosis, or intestinal fibrostenosis. In some embodiments, the sample comprises whole blood, plasma, serum, or biopsy tissue. In some embodiments, the subject is mammal. In some embodiments, the subject is human. In some embodiments, the subject is non-responsive to an induction of anti-Tumor Necrosis Factor (TNF) therapy, or lost response to the anti-TNF therapy after a period of time during treatment. In some embodiments, the inflammatory, fibrostenotic, and/or fibrotic disease is refractory. In some embodiments, the genotype comprises one or more single nucleotide polymorphisms (SNPs) or indels at rs1921622, rs2287037, rs1974675, rs2041739, rs76362690, rs2287037, or rs80256362, a SNP in linkage disequilibrium (LD) therewith, or any combination thereof. In some embodiments, the SNP at rs1921622 comprises an “A” or a “G” on a forward DNA strand encoding the SNP. In some embodiments, the SNP at rs2287037 comprises an “A” or a “G” on a reverse DNA strand encoding the SNP. The method of claim 30, wherein the SNP at rs10213846 comprises a “G” or a “T” allele. In some embodiments, the SNP at rs1974675 comprises a “C” or a “T” on a reverse DNA strand encoding the SNP. In some embodiments, the SNP at rs2041739 comprises an “A” or a “G” on a reverse DNA strand encoding the SNP. In some embodiments, the SNP at rs76362690 comprises an “A” or a “G” on a forward DNA strand encoding the SNP. In some embodiments, the SNP at rs2287037 comprises an “A” or a “G” on a reverse DNA strand encoding the SNP. In some embodiments, the SNP at rs80256362 comprises an “A” or a “G” on a forward DNA strand encoding the SNP. In some embodiments, the Indel at rs1921622 is within SEQ ID NO: 1. In some embodiments, the SNP at rs2287037 is within SEQ ID NO: 2. In some embodiments, the SNP at rs1974675 is within SEQ ID NO: 3. In some embodiments, the SNP at rs2041739 is within s SEQ ID NO: 4. In some embodiments, the SNP at rs76362690 is within SEQ ID NO: 5. In some embodiments, the SNP at rs2287037 is within SEQ ID NO: 6. In some embodiments, the SNP at rs80256362 is within SEQ ID NO: 7. In some embodiments, LD is defined by an r² value of at least 0.80, 0.85, 0.90, 0.95, or 1.0. In some embodiments, the genotype is associated with a risk that a subject has, or will develop, inflammatory bowel disease (IBD), Crohn's disease (CD), or ulcerative colitis (UC), as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰. In some embodiments, the genotype is associated with a risk that the subject has, or will develop, a subclinical phenotype of the disease or condition as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰. In some embodiments, the subclinical phenotype comprises stricturing, penetrating, or stricturing and penetrating, disease phenotypes. In some embodiments, the genotype comprises one or more SNPs in linkage disequilibrium with rs1921622 as determined by an r² value of at least about 0.80, about 0.85, about 0.90, about 0.95, or about 1.0.

Aspects disclosed herein provide methods of determining whether a subject is at risk for developing a disease or condition, in a subject, the method comprising: obtaining a sample from a subject; detecting a presence or an absence of a genotype in the sample obtained from the subject; and determining the subject is at risk for developing the disease or condition, provided the presence of the genotype is detected in the sample obtained from the subject. In some embodiments, the genotype is detected with an assay comprising polymerase chain reaction (PCR), quantitative reverse-transcription PCR (qPCR), automated sequencing, genotype array, or a combination thereof. In some embodiments, wherein the methods further comprise administering to the subject a modulator or IL18R1 activity or expression. In some embodiments, the modulator of IL18R1 activity or expression comprises an agonist or a partial agonist of IL18R1. In some embodiments, the modulator of IL18R1 activity or expression comprises an antagonist or partial antagonist of IL18R1. In some embodiments, the agonist or partial agonist comprises an antibody or antigen-binding fragment, peptide, small molecule. In some embodiments, the modulator of IL18R1 activity or expression comprises an inverse agonist. In some embodiments, the modulator of IL18R1 activity or expression comprises a positive allosteric modulator (PAM). In some embodiments, the modulator of IL18R1 activity or expression comprises a negative allosteric modulator (NAM). In some embodiments, the modulator of IL18R1 activity or expression comprises a small molecule that binds to IL18R1 or IL18, or both. In some embodiments, the modulator of IL18R1 activity or expression comprises an antibody or antigen binding fragment that binds to IL18R1 or IL18, or both. In some embodiments, the modulator of IL18R1 activity or expression comprises recombinant IL18R1 peptide, or a recombinant IL18 peptide. In some embodiments, the modulator of IL18R1 activity or expression comprises a recombinant peptide comprising an amino acid sequence that is about 70%, 75%, 80%, 85%, 90%, 91%, 92%, 93%, 94%, 95%, 96%, 97%, 98%, 99%, or 100% homologous to SEQ ID NO: 8 or SEQ ID NO: 9. In some embodiments, the modulator of IL18R1 activity or expression comprises a recombinant peptide comprising an amino acid sequence that is about 70%, 75%, 80%, 85%, 90%, 91%, 92%, 93%, 94%, 95%, 96%, 97%, 98%, 99%, or 100% homologous to SEQ ID NO: 8 or SEQ ID NO: 9, and wherein the amino acid sequence is truncated at the N-terminal and/or C-terminal ends of the peptide. In some embodiments, the modulator of IL18R1 activity or expression comprises a fusion, a conjugate, or both. In some embodiments, the modulator of IL18R1 activity or expression comprises an agonist or an antagonist of IL18 signaling. In some embodiments, the modulator of IL18R1 activity or expression comprises an agonist or an antagonist of IL18-IL18R1 binding. In some embodiments, the genotype is homozygous or heterozygous. In some embodiments, the disease or condition comprises and inflammatory, fibrostenotic, and/or fibrotic disease or condition. In some embodiments, the inflammatory, fibrostenotic, and/or fibrotic disease or condition comprises inflammatory bowel disease (IBD), Crohn's disease (CD), perianal CD, ulcerative colitis (UC), multiple sclerosis (MS), rheumatoid arthritis (RA), primary sclerosing cholangitis (PSC), Pancolitis, primary billary cihrosis, asthma, Proctitis, Iritis, intestinal fibrosis, pulmonary fibrosis, or intestinal fibrostenosis. In some embodiments, the sample comprises whole blood, plasma, serum, or biopsy tissue. In some embodiments, the subject is mammal. In some embodiments, the subject is human. In some embodiments, the subject is non-responsive to an induction of anti-Tumor Necrosis Factor (TNF) therapy, or lost response to the anti-TNF therapy after a period of time during treatment. In some embodiments, the inflammatory, fibrostenotic, and/or fibrotic disease is refractory. In some embodiments, the genotype comprises one or more single nucleotide polymorphisms (SNPs) or indels at rs1921622, rs2287037, rs1974675, rs2041739, rs76362690, rs2287037, or rs80256362, a SNP in linkage disequilibrium (LD) therewith, or any combination thereof. In some embodiments, the SNP at rs1921622 comprises an “A” or a “G” on a forward DNA strand encoding the SNP. In some embodiments, the SNP at rs2287037 comprises an “A” or a “G” on a reverse DNA strand encoding the SNP. The method of claim 30, wherein the SNP at rs10213846 comprises a “G” or a “T” allele. In some embodiments, the SNP at rs1974675 comprises a “C” or a “T” on a reverse DNA strand encoding the SNP. In some embodiments, the SNP at rs2041739 comprises an “A” or a “G” on a reverse DNA strand encoding the SNP. In some embodiments, the SNP at rs76362690 comprises an “A” or a “G” on a forward DNA strand encoding the SNP. In some embodiments, the SNP at rs2287037 comprises an “A” or a “G” on a reverse DNA strand encoding the SNP. In some embodiments, the SNP at rs80256362 comprises an “A” or a “G” on a forward DNA strand encoding the SNP. In some embodiments, the Indel at rs1921622 is within SEQ ID NO: 1. In some embodiments, the SNP at rs2287037 is within SEQ ID NO: 2. In some embodiments, the SNP at rs1974675 is within SEQ ID NO: 3. In some embodiments, the SNP at rs2041739 is within s SEQ ID NO: 4. In some embodiments, the SNP at rs76362690 is within SEQ ID NO: 5. In some embodiments, the SNP at rs2287037 is within SEQ ID NO: 6. In some embodiments, the SNP at rs80256362 is within SEQ ID NO: 7. In some embodiments, LD is defined by an r² value of at least 0.80, 0.85, 0.90, 0.95, or 1.0. In some embodiments, the genotype is associated with a risk that a subject has, or will develop, inflammatory bowel disease (IBD), Crohn's disease (CD), or ulcerative colitis (UC), as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰. In some embodiments, the genotype is associated with a risk that the subject has, or will develop, a subclinical phenotype of the disease or condition as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰. In some embodiments, the subclinical phenotype comprises stricturing, penetrating, or stricturing and penetrating, disease phenotypes. In some embodiments, the genotype comprises one or more SNPs in linkage disequilibrium with rs1921622 as determined by an r² value of at least about 0.80, about 0.85, about 0.90, about 0.95, or about 1.0.

Aspects disclosed herein provide methods of determining whether a subject is suitable for treatment of a disease or condition with a modulator of Interleukin 18 Receptor 1(IL18R1) activity or expression, the method comprising: obtaining a sample from a subject; detecting a presence or an absence of a genotype in the sample obtained from the subject; and determining the subject is suitable for treatment of the disease or condition with a modulator of IL18R1, provided the presence of the genotype is detected in the sample obtained from the subject. In some embodiments, the genotype is detected with an assay comprising polymerase chain reaction (PCR), quantitative reverse-transcription PCR (qPCR), automated sequencing, genotype array, or a combination thereof. In some embodiments, wherein the methods further comprise administering to the subject a modulator or IL18R1 activity or expression. In some embodiments, the modulator of IL18R1 activity or expression comprises an agonist or a partial agonist of IL18R1. In some embodiments, the modulator of IL18R1 activity or expression comprises an antagonist or partial antagonist of IL18R1. In some embodiments, the agonist or partial agonist comprises an antibody or antigen-binding fragment, peptide, small molecule. In some embodiments, the modulator of IL18R1 activity or expression comprises an inverse agonist. In some embodiments, the modulator of IL18R1 activity or expression comprises a positive allosteric modulator (PAM). In some embodiments, the modulator of IL18R1 activity or expression comprises a negative allosteric modulator (NAM). In some embodiments, the modulator of IL18R1 activity or expression comprises a small molecule that binds to IL18R1 or IL18, or both. In some embodiments, the modulator of IL18R1 activity or expression comprises an antibody or antigen binding fragment that binds to IL18R1 or IL18, or both. In some embodiments, the modulator of IL18R1 activity or expression comprises recombinant IL18R1 peptide, or a recombinant IL18 peptide. In some embodiments, the modulator of IL18R1 activity or expression comprises a recombinant peptide comprising an amino acid sequence that is about 70%, 75%, 80%, 85%, 90%, 91%, 92%, 93%, 94%, 95%, 96%, 97%, 98%, 99%, or 100% homologous to SEQ ID NO: 8 or SEQ ID NO: 9. In some embodiments, the modulator of IL18R1 activity or expression comprises a recombinant peptide comprising an amino acid sequence that is about 70%, 75%, 80%, 85%, 90%, 91%, 92%, 93%, 94%, 95%, 96%, 97%, 98%, 99%, or 100% homologous to SEQ ID NO: 8 or SEQ ID NO: 9, and wherein the amino acid sequence is truncated at the N-terminal and/or C-terminal ends of the peptide. In some embodiments, the modulator of IL18R1 activity or expression comprises a fusion, a conjugate, or both. In some embodiments, the modulator of IL18R1 activity or expression comprises an agonist or an antagonist of IL18 signaling. In some embodiments, the modulator of IL18R1 activity or expression comprises an agonist or an antagonist of IL18-IL18R1 binding. In some embodiments, the genotype is homozygous or heterozygous. In some embodiments, the disease or condition comprises and inflammatory, fibrostenotic, and/or fibrotic disease or condition. In some embodiments, the inflammatory, fibrostenotic, and/or fibrotic disease or condition comprises inflammatory bowel disease (IBD), Crohn's disease (CD), perianal CD, ulcerative colitis (UC), multiple sclerosis (MS), rheumatoid arthritis (RA), primary sclerosing cholangitis (PSC), Pancolitis, primary billary cihrosis, asthma, Proctitis, Iritis, intestinal fibrosis, pulmonary fibrosis, or intestinal fibrostenosis. In some embodiments, the sample comprises whole blood, plasma, serum, or biopsy tissue. In some embodiments, the subject is mammal. In some embodiments, the subject is human. In some embodiments, the subject is non-responsive to an induction of anti-Tumor Necrosis Factor (TNF) therapy, or lost response to the anti-TNF therapy after a period of time during treatment. In some embodiments, the inflammatory, fibrostenotic, and/or fibrotic disease is refractory. In some embodiments, the genotype comprises one or more single nucleotide polymorphisms (SNPs) or indels at rs1921622, rs2287037, rs1974675, rs2041739, rs76362690, rs2287037, or rs80256362, a SNP in linkage disequilibrium (LD) therewith, or any combination thereof. In some embodiments, the SNP at rs1921622 comprises an “A” or a “G” on a forward DNA strand encoding the SNP. In some embodiments, the SNP at rs2287037 comprises an “A” or a “G” on a reverse DNA strand encoding the SNP. The method of claim 30, wherein the SNP at rs10213846 comprises a “G” or a “T” allele. In some embodiments, the SNP at rs1974675 comprises a “C” or a “T” on a reverse DNA strand encoding the SNP. In some embodiments, the SNP at rs2041739 comprises an “A” or a “G” on a reverse DNA strand encoding the SNP. In some embodiments, the SNP at rs76362690 comprises an “A” or a “G” on a forward DNA strand encoding the SNP. In some embodiments, the SNP at rs2287037 comprises an “A” or a “G” on a reverse DNA strand encoding the SNP. In some embodiments, the SNP at rs80256362 comprises an “A” or a “G” on a forward DNA strand encoding the SNP. In some embodiments, the Indel at rs1921622 is within SEQ ID NO: 1. In some embodiments, the SNP at rs2287037 is within SEQ ID NO: 2. In some embodiments, the SNP at rs1974675 is within SEQ ID NO: 3. In some embodiments, the SNP at rs2041739 is within s SEQ ID NO: 4. In some embodiments, the SNP at rs76362690 is within SEQ ID NO: 5. In some embodiments, the SNP at rs2287037 is within SEQ ID NO: 6. In some embodiments, the SNP at rs80256362 is within SEQ ID NO: 7. In some embodiments, LD is defined by an r² value of at least 0.80, 0.85, 0.90, 0.95, or 1.0. In some embodiments, the genotype is associated with a risk that a subject has, or will develop, inflammatory bowel disease (IBD), Crohn's disease (CD), or ulcerative colitis (UC), as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰. In some embodiments, the genotype is associated with a risk that the subject has, or will develop, a subclinical phenotype of the disease or condition as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰. In some embodiments, the subclinical phenotype comprises stricturing, penetrating, or stricturing and penetrating, disease phenotypes. In some embodiments, the genotype comprises one or more SNPs in linkage disequilibrium with rs1921622 as determined by an r² value of at least about 0.80, about 0.85, about 0.90, about 0.95, or about 1.0.

Aspects disclosed herein provide methods for processing or analyzing a sample obtained from a subject, the method comprising: obtaining a sample from a subject; subjecting the sample to an assay by sequencing, genotype array, and/or nucleic acid amplification, to yield a data set comprising data corresponding to a presence or an absence of a genotype; in a programmed computer, inputting said data from (b) to a trained algorithm to determine whether the subject is at risk of developing, a disease or disorder, wherein the trained algorithm is trained with a plurality of training samples, and wherein said sample is independent of said plurality of training samples; and electronically outputting a report comprising the determination for the subject. In some embodiments, wherein (c) comprises calculating a polygenic risk score (PRS), and the PRS comprises a normalized weighted sum of a number of risk alleles within the genotype present in the subject with weights proportional to a beta value of association between the genotype with the disease or condition. In some embodiments, the data set of (b) further comprises data corresponding to a presence or an absence of a surrogate genotype, provided an absence of a genotype is detected. In some embodiments, the surrogate genotype is in linkage disequilibrium with the absent genotype as determined by an r² value of at least about, 0.8, about 0.85, about 0.90, about 0.95, or about 1.0. In some embodiments, the report is configured to display the determination of the subject on a user interface of an electronic device. In some embodiments, the electronic device comprises a personal electronic device belonging to the subject. In some embodiments, the methods comprise administering to the subject a modulator or IL18R1 activity or expression, provided the subject is determined to be at risk of having, or developing, the disease or condition. In some embodiments, the modulator of IL18R1 activity or expression comprises an agonist or a partial agonist of IL18R1. In some embodiments, the modulator of IL18R1 activity or expression comprises an antagonist or partial antagonist of IL18R1. In some embodiments, the agonist or partial agonist comprises an antibody or antigen-binding fragment, peptide, small molecule. In some embodiments, the modulator of IL18R1 activity or expression comprises an inverse agonist. In some embodiments, the modulator of IL18R1 activity or expression comprises a positive allosteric modulator (PAM). In some embodiments, the modulator of IL18R1 activity or expression comprises a negative allosteric modulator (NAM). In some embodiments, the modulator of IL18R1 activity or expression comprises a small molecule that binds to IL18R1 or IL18, or both. In some embodiments, the modulator of IL18R1 activity or expression comprises an antibody or antigen binding fragment that binds to IL18R1 or IL18, or both. In some embodiments, the modulator of IL18R1 activity or expression comprises recombinant IL18R1 peptide, or a recombinant IL18 peptide. In some embodiments, the modulator of IL18R1 activity or expression comprises a recombinant peptide comprising an amino acid sequence that is about 70%, 75%, 80%, 85%, 90%, 91%, 92%, 93%, 94%, 95%, 96%, 97%, 98%, 99%, or 100% homologous to SEQ ID NO: 8 or SEQ ID NO: 9. In some embodiments, the modulator of IL18R1 activity or expression comprises a recombinant peptide comprising an amino acid sequence that is about 70%, 75%, 80%, 85%, 90%, 91%, 92%, 93%, 94%, 95%, 96%, 97%, 98%, 99%, or 100% homologous to SEQ ID NO: 8 or SEQ ID NO: 9, and wherein the amino acid sequence is truncated at the N-terminal and/or C-terminal ends of the peptide. In some embodiments, the modulator of IL18R1 activity or expression comprises a fusion, a conjugate, or both. In some embodiments, the modulator of IL18R1 activity or expression comprises an agonist or an antagonist of IL18 signaling. In some embodiments, the modulator of IL18R1 activity or expression comprises an agonist or an antagonist of IL18-IL18R1 binding. In some embodiments, the genotype is homozygous or heterozygous. In some embodiments, the disease or condition comprises and inflammatory, fibrostenotic, and/or fibrotic disease or condition. In some embodiments, the inflammatory, fibrostenotic, and/or fibrotic disease or condition comprises inflammatory bowel disease (IBD), Crohn's disease (CD), perianal CD, ulcerative colitis (UC), multiple sclerosis (MS), rheumatoid arthritis (RA), primary sclerosing cholangitis (PSC), Pancolitis, primary billary cihrosis, asthma, Proctitis, Iritis, intestinal fibrosis, pulmonary fibrosis, or intestinal fibrostenosis. In some embodiments, the sample comprises whole blood, plasma, serum, or biopsy tissue. In some embodiments, the subject is mammal. In some embodiments, the subject is human. In some embodiments, the subject is non-responsive to an induction of anti-Tumor Necrosis Factor (TNF) therapy, or lost response to the anti-TNF therapy after a period of time during treatment. In some embodiments, the inflammatory, fibrostenotic, and/or fibrotic disease is refractory. In some embodiments, the genotype comprises one or more single nucleotide polymorphisms (SNPs) or indels at rs1921622, rs2287037, rs1974675, rs2041739, rs76362690, rs2287037, or rs80256362, a SNP in linkage disequilibrium (LD) therewith, or any combination thereof. In some embodiments, the SNP at rs1921622 comprises an “A” or a “G” on a forward DNA strand encoding the SNP. In some embodiments, the SNP at rs2287037 comprises an “A” or a “G” on a reverse DNA strand encoding the SNP. The method of claim 30, wherein the SNP at rs10213846 comprises a “G” or a “T” allele. In some embodiments, the SNP at rs1974675 comprises a “C” or a “T” on a reverse DNA strand encoding the SNP. In some embodiments, the SNP at rs2041739 comprises an “A” or a “G” on a reverse DNA strand encoding the SNP. In some embodiments, the SNP at rs76362690 comprises an “A” or a “G” on a forward DNA strand encoding the SNP. In some embodiments, the SNP at rs2287037 comprises an “A” or a “G” on a reverse DNA strand encoding the SNP. In some embodiments, the SNP at rs80256362 comprises an “A” or a “G” on a forward DNA strand encoding the SNP. In some embodiments, the Indel at rs1921622 is within SEQ ID NO: 1. In some embodiments, the SNP at rs2287037 is within SEQ ID NO: 2. In some embodiments, the SNP at rs1974675 is within SEQ ID NO: 3. In some embodiments, the SNP at rs2041739 is within s SEQ ID NO: 4. In some embodiments, the SNP at rs76362690 is within SEQ ID NO: 5. In some embodiments, the SNP at rs2287037 is within SEQ ID NO: 6. In some embodiments, the SNP at rs80256362 is within SEQ ID NO: 7. In some embodiments, LD is defined by an r² value of at least 0.80, 0.85, 0.90, 0.95, or 1.0. In some embodiments, the genotype is associated with a risk that a subject has, or will develop, inflammatory bowel disease (IBD), Crohn's disease (CD), or ulcerative colitis (UC), as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰. In some embodiments, the genotype is associated with a risk that the subject has, or will develop, a subclinical phenotype of the disease or condition as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰. In some embodiments, the subclinical phenotype comprises stricturing, penetrating, or stricturing and penetrating, disease phenotypes. In some embodiments, the genotype comprises one or more SNPs in linkage disequilibrium with rs1921622 as determined by an r² value of at least about 0.80, about 0.85, about 0.90, about 0.95, or about 1.0. In some embodiments, the genotype comprises at least about 1, 2, 3, 4, 5, 6, 7, 8, 9, 10, 11, 12, 13, or 14, single nucleotide polymorphism (SNP) or indels.

Aspects disclosed herein provide methods for processing or analyzing a sample obtained from a subject, the method comprising: obtaining a sample from a subject; subjecting the sample to an assay by sequencing, genotype array, and/or nucleic acid amplification, to yield a data set comprising data corresponding to a presence or an absence of a genotype; in a programmed computer, inputting said data from (b) to a trained algorithm to determine a likelihood that the subject is suitable for treatment of a disease or disorder with an agonist of IL18R1, wherein the trained algorithm is trained with a plurality of training samples, and wherein said sample is independent of said plurality of training samples; and electronically outputting a report comprising the determination for the subject. In some embodiments, wherein (c) comprises calculating a polygenic risk score (PRS), and the PRS comprises a normalized weighted sum of a number of risk alleles within the genotype present in the subject with weights proportional to a beta value of association between the genotype with the disease or condition. In some embodiments, the data set of (b) further comprises data corresponding to a presence or an absence of a surrogate genotype, provided an absence of a genotype is detected. In some embodiments, the surrogate genotype is in linkage disequilibrium with the absent genotype as determined by an r² value of at least about, 0.8, about 0.85, about 0.90, about 0.95, or about 1.0. In some embodiments, the report is configured to display the determination of the subject on a user interface of an electronic device. In some embodiments, the electronic device comprises a personal electronic device belonging to the subject. In some embodiments, the methods comprise administering to the subject a modulator or IL18R1 activity or expression, provided the subject is determined to be at risk of having, or developing, the disease or condition. In some embodiments, the modulator of IL18R1 activity or expression comprises an agonist or a partial agonist of IL18R1. In some embodiments, the modulator of IL18R1 activity or expression comprises an antagonist or partial antagonist of IL18R1. In some embodiments, the agonist or partial agonist comprises an antibody or antigen-binding fragment, peptide, small molecule. In some embodiments, the modulator of IL18R1 activity or expression comprises an inverse agonist. In some embodiments, the modulator of IL18R1 activity or expression comprises a positive allosteric modulator (PAM). In some embodiments, the modulator of IL18R1 activity or expression comprises a negative allosteric modulator (NAM). In some embodiments, the modulator of IL18R1 activity or expression comprises a small molecule that binds to IL18R1 or IL18, or both. In some embodiments, the modulator of IL18R1 activity or expression comprises an antibody or antigen binding fragment that binds to IL18R1 or IL18, or both. In some embodiments, the modulator of IL18R1 activity or expression comprises recombinant IL18R1 peptide, or a recombinant IL18 peptide. In some embodiments, the modulator of IL18R1 activity or expression comprises a recombinant peptide comprising an amino acid sequence that is about 70%, 75%, 80%, 85%, 90%, 91%, 92%, 93%, 94%, 95%, 96%, 97%, 98%, 99%, or 100% homologous to SEQ ID NO: 8 or SEQ ID NO: 9. In some embodiments, the modulator of IL18R1 activity or expression comprises a recombinant peptide comprising an amino acid sequence that is about 70%, 75%, 80%, 85%, 90%, 91%, 92%, 93%, 94%, 95%, 96%, 97%, 98%, 99%, or 100% homologous to SEQ ID NO: 8 or SEQ ID NO: 9, and wherein the amino acid sequence is truncated at the N-terminal and/or C-terminal ends of the peptide. In some embodiments, the modulator of IL18R1 activity or expression comprises a fusion, a conjugate, or both. In some embodiments, the modulator of IL18R1 activity or expression comprises an agonist or an antagonist of IL18 signaling. In some embodiments, the modulator of IL18R1 activity or expression comprises an agonist or an antagonist of IL18-IL18R1 binding. In some embodiments, the genotype is homozygous or heterozygous. In some embodiments, the disease or condition comprises and inflammatory, fibrostenotic, and/or fibrotic disease or condition. In some embodiments, the inflammatory, fibrostenotic, and/or fibrotic disease or condition comprises inflammatory bowel disease (IBD), Crohn's disease (CD), perianal CD, ulcerative colitis (UC), multiple sclerosis (MS), rheumatoid arthritis (RA), primary sclerosing cholangitis (PSC), Pancolitis, primary billary cihrosis, asthma, Proctitis, Iritis, intestinal fibrosis, pulmonary fibrosis, or intestinal fibrostenosis. In some embodiments, the sample comprises whole blood, plasma, serum, or biopsy tissue. In some embodiments, the subject is mammal. In some embodiments, the subject is human. In some embodiments, the subject is non-responsive to an induction of anti-Tumor Necrosis Factor (TNF) therapy, or lost response to the anti-TNF therapy after a period of time during treatment. In some embodiments, the inflammatory, fibrostenotic, and/or fibrotic disease is refractory. In some embodiments, the genotype comprises one or more single nucleotide polymorphisms (SNPs) or indels at rs1921622, rs2287037, rs1974675, rs2041739, rs76362690, rs2287037, or rs80256362, a SNP in linkage disequilibrium (LD) therewith, or any combination thereof. In some embodiments, the SNP at rs1921622 comprises an “A” or a “G” on a forward DNA strand encoding the SNP. In some embodiments, the SNP at rs2287037 comprises an “A” or a “G” on a reverse DNA strand encoding the SNP. The method of claim 30, wherein the SNP at rs10213846 comprises a “G” or a “T” allele. In some embodiments, the SNP at rs1974675 comprises a “C” or a “T” on a reverse DNA strand encoding the SNP. In some embodiments, the SNP at rs2041739 comprises an “A” or a “G” on a reverse DNA strand encoding the SNP. In some embodiments, the SNP at rs76362690 comprises an “A” or a “G” on a forward DNA strand encoding the SNP. In some embodiments, the SNP at rs2287037 comprises an “A” or a “G” on a reverse DNA strand encoding the SNP. In some embodiments, the SNP at rs80256362 comprises an “A” or a “G” on a forward DNA strand encoding the SNP. In some embodiments, the Indel at rs1921622 is within SEQ ID NO: 1. In some embodiments, the SNP at rs2287037 is within SEQ ID NO: 2. In some embodiments, the SNP at rs1974675 is within SEQ ID NO: 3. In some embodiments, the SNP at rs2041739 is within s SEQ ID NO: 4. In some embodiments, the SNP at rs76362690 is within SEQ ID NO: 5. In some embodiments, the SNP at rs2287037 is within SEQ ID NO: 6. In some embodiments, the SNP at rs80256362 is within SEQ ID NO: 7. In some embodiments, LD is defined by an r² value of at least 0.80, 0.85, 0.90, 0.95, or 1.0. In some embodiments, the genotype is associated with a risk that a subject has, or will develop, inflammatory bowel disease (IBD), Crohn's disease (CD), or ulcerative colitis (UC), as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰. In some embodiments, the genotype is associated with a risk that the subject has, or will develop, a subclinical phenotype of the disease or condition as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰. In some embodiments, the subclinical phenotype comprises stricturing, penetrating, or stricturing and penetrating, disease phenotypes. In some embodiments, the genotype comprises one or more SNPs in linkage disequilibrium with rs1921622 as determined by an r² value of at least about 0.80, about 0.85, about 0.90, about 0.95, or about 1.0. In some embodiments, the genotype comprises at least about 1, 2, 3, 4, 5, 6, 7, 8, 9, 10, 11, 12, 13, or 14, single nucleotide polymorphism (SNP) or indels.

Aspects disclosed herein provide methods of treating a subject in need thereof with a modulator of interleukin 18 receptor 1 (IL18R1) activity or expression, wherein the subject has moderate to severe Crohn's disease (CD), and wherein the subject has a genotype characterized by the presence of one or more SNPs. In some embodiments, the one or more SNPs comprises a SNP listed in Table 1. In some embodiments, the single nucleotide polymorphism is associated with structuring. In some embodiments, the stricturing is isolated to an ileocolonic region of an intestine. In some embodiments, the one or more SNPs comprises a SNP listed in Table 2. In some embodiments, the single nucleotide polymorphism is associated with a risk of a subject developing morphological defects in ileal Paneth cells. In some embodiments, the one or more SNPs comprises a SNP listed in Table 3.

Aspects disclosed herein provide methods of treating a subject in need thereof with a modulator of interleukin 18 receptor 1 (IL18R1) activity or expression, wherein the subject has moderate to severe inflammatory bowel disease (IBD), and wherein the subject has a genotype characterized by the presence of one or more SNPs. In some embodiments, the one or more SNPs comprises a SNP listed in Table 4.

Aspects disclosed herein provide methods of treating a subject in need thereof with a modulator of interleukin 18 receptor 1 (IL18R1) activity or expression, wherein the subject has moderate to severe ulcerative colitis, and wherein the subject has a genotype characterized by the presence of one or more SNPs. In some embodiments, the one or more SNPs comprises a SNP listed in Table 5.

### BRIEF DESCRIPTION OF THE DRAWINGS

FIG. 1A to FIG. 1QQQQQQQ illustrate meta-analysis of IL18R1 single nucleotide polymorphism in association with Crohn's disease (CD), inflammatory bowel disease (IBD), or ulcerative colitis (UC), and various subclinical phenotypes of CD, IBD, and UC.

### DETAILED DESCRIPTION OF THE DISCLOSURE

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### Certain Terminologies

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value.

As used herein “consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed disclosure, such as compositions for treating skin disorders like acne, eczema, psoriasis, and rosacea.

The terms “homologous,” “homology,” or “percent homology” are used herein to generally mean an amino acid sequence or a nucleic acid sequence having the same, or similar sequence to a reference sequence. Percent homology of sequences can be determined using the most recent version of BLAST, as of the filing date of this application.

The terms “increased,” or “increase” are used herein to generally mean an increase by a statically significant amount. In some embodiments, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms, “decreased” or “decrease” are used herein generally to mean a decrease by a statistically significant amount. In some embodiments, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

The term “subject” encompasses mammals. Non-limiting examples of mammal include, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human. The term “animal” as used herein comprises human beings and non-human animals. In one embodiment, a “non-human animal” is a mammal, for example a rodent such as rat or a mouse.

The term “gene,” as used herein, refers to a segment of nucleic acid that encodes an individual protein or RNA (also referred to as a “coding sequence” or “coding region”), optionally together with associated regulatory region such as promoter, operator, terminator and the like, which may be located upstream or downstream of the coding sequence.

The term “genetic variant” as used herein refers to an aberration in (e.g., a mutation), or of (e.g., copy number variation), a nucleic acid sequence, as compared to the nucleic acid sequence in a reference population. In some embodiments, the genetic variant is common in the reference population. In some embodiments, the genetic variant is rare in the reference population.

The term, “genotype” as disclosed herein, refers to the chemical composition of polynucleotide sequences within the genome of an individual. In some embodiments, the genotype comprises a single nucleotide polymorphism (SNP), or and indel (insertion or deletion, of a nucleobase within a polynucleotide sequence). In some embodiments, a genotype for a particular SNP, or indel is heterozygous. In some embodiments, a genotype for a particular SNP, or indel is homozygous.

The term, “single nucleotide polymorphism” or “SNP,” as disclosed herein, refers to a variation in a single nucleotide within a polynucleotide sequence. The variation of an SNV may have multiple different forms. The usage of the term “single nucleotide polymorphism” or “SNP” should not imply any limit on the frequency with which each variation occurs. A single form of an SNP is referred to as an “allele.” An SNP can be mono-, bi-, tri, or tetra-allelic. An SNP may include a “risk allele,” a “protective allele,” or neither. By way of example, a reference polynucleotide sequence reading 5′ to 3′ is TTACG. A SNP at allele position 3 (of 5′-TTACG-3′) comprise a substitution of the reference allele, “A” to a non-reference allele, “C.” If the “C” allele of the SNP is associated with an increased probability of developing a phenotypic trait, the allele is considered a “risk” allele. However, the same SNP may also comprise a substitution of the “A” allele to a “T” allele at position 3. If the T allele of the SNP is associated with a decreased probability of developing a phenotypic trait, the allele is considered a “protective” allele. In some embodiments, the SNP is represented by an “rs” number, which refers to the accession of reference cluster of one more submitted SNPs in the dbSNP bioinformatics database as of the filing date of this patent application, and which is included within a sequence that comprises the total number of nucleobases from 5′ to 3′. In some embodiments, a SNP may be further defined by the position of the SNP (nucleobase) within the dbSNP sequence, the position of which is always with reference to 5′ length of the sequence plus 1. In some embodiments, a SNP is defined as the genomic position in a reference genome and the allele change (e.g. chromosome 7 at position 234,123,567 from G allele to A allele in the reference human genome build 37). In some embodiments, the SNP is defined as the genomic position identified with [brackets] or an “N” in a sequence disclosed herein.

The term, “indel,” as disclosed herein, refers to an insertion, or a deletion, of a nucleobase within a polynucleotide sequence. An indel can be mono-, bi-, tri, or tetra-allelic. An indel may be “risk,” a “protective,” or neither, for a phenotypic trait. In some embodiments, the indel is represented by an “rs” number, which refers to the accession of reference cluster of one more submitted indels in the dbSNP bioinformatics database as of the filing date of this patent application, and which is included in a sequence that comprises the total number of nucleobases from 5′ to 3′. In some embodiments, an indel may be further defined by the position of the insertion/deletion within the dbSNP sequence, the position of which is always with reference to the 5′ length of the sequence plus 1. In some embodiments, an indel is defined as the genomic position in a reference genome and the allele change. In some embodiments, the indel is defined as the genomic position identified with [brackets] or an “N” in a sequence disclosed herein.

“Haplotype” as used herein, encompasses a group of one or more genotypes, SNPs, or indels, which tend to be inherited together in a reference population. In some embodiments, a haplotype comprises particular SNPs, or indels, and any SNP, or indel in linkage disequilibrium therewith.

“Linkage disequilibrium,” or “LD,” as used herein refers to the non-random association of alleles or indels in different gene loci in a given population. LD may be defined by a D′ value corresponding to the difference between an observed and expected allele or indel frequencies in the population (D=Pab−PaPb), which is scaled by the theoretical maximum value of D. LD may be defined by an r² value corresponding to the difference between an observed and expected unit of risk frequencies in the population (D=Pab−PaPb), which is scaled by the individual frequencies of the different loci. In some embodiments, D′ comprises at least 0.20. In some embodiments, r² comprises at least 0.70.

The terms “treat,” “treating,” and “treatment” as used herein refers to alleviating or abrogating a disorder, disease, or condition; or one or more of the symptoms associated with the disorder, disease, or condition; or alleviating or eradicating a cause of the disorder, disease, or condition itself. Desirable effects of treatment can include, but are not limited to, preventing occurrence or recurrence of disease, alleviation of symptoms, diminishing any direct or indirect pathological consequences of the disease, preventing metastasis, decreasing the rate of disease progression, amelioration or palliation of the disease state and remission or improved prognosis.

The term “therapeutically effective amount” refers to the amount of a compound or therapy that, when administered, is sufficient to prevent development of, or alleviate to some extent, one or more of the symptoms of a disorder, disease, or condition of the disease; or the amount of a compound that is sufficient to elicit biological or medical response of a cell, tissue, system, animal, or human that is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. A component can be “pharmaceutically acceptable” in the sense of being compatible with the other ingredients of a pharmaceutical formulation. It can also be suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004).

The term “pharmaceutical composition” refers to a mixture of a compound disclosed herein with other chemical components, such as diluents or carriers. The pharmaceutical composition can facilitate administration of the compound to an organism. Multiple techniques of administering a compound exist in the art including, but not limited to, oral, injection, aerosol, parenteral, and topical administration.

The term “inflammatory bowel disease” or “IBD” as used herein refers to gastrointestinal disorders of the gastrointestinal tract. Non-limiting examples of IBD include, Crohn's disease (CD), ulcerative colitis (UC), indeterminate colitis (IC), microscopic colitis, diversion colitis, Behcet's disease, and other inconclusive forms of IBD. In some instances, IBD comprises fibrosis, fibrostenosis, stricturing and/or penetrating disease, obstructive disease, or a disease that is refractory (e.g., mrUC, refractory CD), perianal CD, or other complicated forms of IBD.

Non-limiting examples of “sample” include any material from which nucleic acids and/or proteins can be obtained. As non-limiting examples, this includes whole blood, peripheral blood, plasma, serum, saliva, mucus, urine, semen, lymph, fecal extract, cheek swab, cells or other bodily fluid or tissue, including but not limited to tissue obtained through surgical biopsy or surgical resection. In various embodiments, the sample comprises tissue from the large and/or small intestine. In various embodiments, the large intestine sample comprises the cecum, colon (the ascending colon, the transverse colon, the descending colon, and the sigmoid colon), rectum and/or the anal canal. In some embodiments, the small intestine sample comprises the duodenum, jejunum, and/or the ileum. Alternatively, a sample can be obtained through primary patient derived cell lines, or archived patient samples in the form of preserved samples, or fresh frozen samples.

The term “biomarker” comprises a measurable substance in a subject whose presence, level, or activity, is indicative of a phenomenon (e.g., phenotypic expression or activity; disease, condition, subclinical phenotype of a disease or condition, infection; or environmental stimuli). In some embodiments, a biomarker comprises a gene, or gene expression product. In some embodiments, a biomarker comprises a cytokine (e.g., IL-1α, IL-1β, IL-2, IL-3, IL-4, IL-5, IL-6, IL-8, IL-9, IL-10, IL-13, IL-17, IL-17F, IL-22, TNF-α, TNF-β, IFN-α1/-α2, IFN-β, IFN-γ, TNFSF superfamily: TNF, TL1A, FasL, LIGHT, TRAIL, and TWEAK). In some embodiments, a biomarker comprises a cell type (e.g., Natural Killer (NK) cells, T cells, Effector T cells (Teff), Regulatory T cells (Treg) B cells, T helper (Th) cells, cluster of differentiation (CD) cells, innate lymphoid cells (ILC), antigen-presenting cells (APC), monocytes Paneth cells, granulocytes, dendritic cells, and macrophages).

The term “serological marker,” as used herein refers to a type of biomarker representing an antigenic response in a subject that may be detected in the serum of the subject. In some embodiments, a serological comprises an antibody against various fungal antigens. Non-limiting examples of a serological marker comprise anti-Saccharomyces cerevisiae antibody (ASCA), an anti-neutrophil cytoplasmic antibody (ANCA), E. coli outer membrane porin protein C (OmpC), anti-Malassezia restricta antibody, anti-Malassezia pachydermatis antibody, anti-Malassezia furfur antibody, anti-Malassezia globasa antibody, anti-Cladosporium albicans antibody, anti-laminaribiose antibody (ALCA), anti-chitobioside antibody (ACCA), anti-laminarin antibody, anti-chitin antibody, pANCA antibody, anit-I2 antibody, and anti-Cbirl flagellin antibody.

The term “microbiome” and its variation used herein describe the populations and interactions of the bacteria, fungi, protists, and virus that align the gastrointestinal tract of a subject. A subject afflicted with IBD may possess presence, absence, excess, diminished, or a combination thereof of a microbiome s compared to a healthy subject. Non-limiting examples of bacteria associated with IBD includes strains, sub-strains, and enterotypes of enterobacteriacease, pasteurellaceae, fusobacteriacease, neisseriaceae, veillonellaceae, gemellaceae, bacteriodales, clostridales, erysipelotrichaeceae, bifidobacteriaceae Bacteroides, Faecalibacterium, Roseburia, Blautia, Ruminococcus, Coprococcus, Streptococcus, Dorea, Blautia, Ruminococcus, Lactobacillus, Enterococcus, Streptococcus, Escherichia coli, Fusobacterium nucleatum, Haemophilus parainfluenzae (pasteurellaceae), Veillonella parvula, Eikenella corrodens (neisseriaceae), and Gemella moribillum, Bacteroides vulgatus, Bacteroides caccae, Bifidobacterium bifidum, Bifidobacterium longum, Bifidobacterium adolescentis, Bifidobacterium dentum, Blautia hansenii, Ruminococcus gnavus, Clostridium nexile, Faecalibacterium prausnitzii, Ruminococcus torques, Clostridium bolteae, Eubacterium rectale, Roseburia intestinalis, Coprococcus comes, Actinomyces, Lactococcus, Roseburia, Streptococcus, Blautia, Dialister, Desulfovibrio, Escherichia, Lactobacillus, Coprococcus, Clostridium, Bifidobacterium, Klebsiella, Granulicatella, Eubacterium, Anaerostipes, Parabacteroides, Coprobacillus, Gordonibacter, Collinsella, Bacteroides, Faecalibacterium, Anaerotruncus, Alistipes, Haemophilus, Anaerococcus, Veillonella, Arevotella, Akkermansia, Bilophila, Sutterella, Eggerthella, Holdemania, Gemella, Peptoniphilus, Rothia, Enterococcus, Pediococcus, Citrobacter, Odoribacter, Enterobacteria, Fusobacterium, and Proteus. Non-limiting examples of viruses associated with IBD include picovirinae, Lactococcus phage, Cellulophaga phage, Bacteroides phage, C2 like virus, Enterococcus phage, caudivurales, Cellulophaga phage, phiCD119 like virus, Croceibacter phage, Clostridium phage, spounavirinae, Riemerella phage, lambda like virus, Bacillus phage, terenvirinae, Lactobacillus phage, Enterobacteria phage, Thermoanaerobacterium phage, Strepcoccus phage, and Pseudomonas phage. Non-limiting examples of fungi genera associated with IBD includes Malassezia, Cladosporium, Aureobasidium, Fusarium, Candida, Pichia, Saccharomyces, and Escherichia.

The term “medically refractory,” or “refractory,” as used herein, refers to the failure of a standard treatment to induce remission of a disease. In some embodiments, the disease comprises an inflammatory disease disclosed herein. A non-limiting example of refractory inflammatory disease includes refractory Crohn's disease, and refractory ulcerative colitis (e.g., mrUC). Non-limiting examples of standard treatment include glucocorticosteriods, anti-TNF therapy, anti-a4-b7 therapy (vedolizumab), anti-IL12p40 therapy (ustekinumab), Thalidomide, and Cytoxin.

The term “anti-tumor necrosis factor (TNF) non-response,” or “anti-TNF non-response,” as used herein, refers to a subject not responding to the induction of an anti-TNF therapy (primary non-response), or loss of response during maintenance after a successful induction of the anti-TNF therapy (secondary loss of response). In some embodiments, the induction of the anti-TNF therapy comprises 1, 2, 3, 4, or 5, doses of the therapy. In some embodiments, loss of response is characterized by a reappearance of symptoms consistent with a flare after an initial response to the anti-TNF therapy.

### Methods

Disease or Condition

Aspects disclosed herein provide methods of treating, diagnosing, prognosing, or monitoring, a disease or condition. In some cases, the disease or condition comprises an inflammatory disease, fibrostenotic disease, and/or fibrotic disease. Non-limiting examples of inflammatory diseases include diseases of the gastrointestinal (GI) tract, liver, gallbladder, and joints. In some cases, the inflammatory disease inflammatory bowel disease (IBD), Crohn's disease (CD), or ulcerative colitis, systemic lupus erythematosus (SLE), multiple sclerosis (MS), asthma, celiac disease, primary billary cihrosis (PBC), or rheumatoid arthritis. A subject may suffer from fibrosis, fibrostenosis, or a fibrotic disease, either isolated or in combination with an inflammatory disease. An exemplary fibrotic disease is primary sclerosing cholangitis (PSC).

In some instances, the disease or condition is refractory, which refers a quality of the disease or condition such that there is an observed failure of a standard treatment to induce remission of a disease or condition. Non-limiting examples of refractory inflammatory disease include refractory Crohn's disease, and medically refractory ulcerative colitis (e.g., mrUC). Non-limiting examples of standard treatment include glucocorticosteriods, anti-TNF therapy, anti-a4-b7 therapy (vedolizumab), anti-IL12p40 therapy (ustekinumab), Thalidomide, and Cytoxin. In some instances, the refractory disease or condition is characterized by an increase in colitis, inflammation, fibrosis, fibrostenosis, stricturing, penetrating, obstructive, or otherwise complicated, disease of the GI tract.

Subject

Disclosed herein, in some embodiments, are methods of treating, diagnosing, prognosing, or monitoring, a disease or condition in a subject. In some instances, the subject is a mammal. In some embodiments, the subject comprises a mouse, rat, guinea pig, rabbit, chimpanzee, or farm animal. In some instances, the subject is human. In some instances, the subject is diagnosed with the disease or condition disclosed herein. Non-limiting methods for diagnosis using existing indices and scoring systems include Crohn's Disease Activity Index (CDAI), Ulcerative Colitis Disease Activity Index (UCDAI), guidelines from American College of Gastroenterology (ACG) and European Crohn's and Colitis Organization (ECCO), patient-reported outcomes (PRO-2), Harvey-Bradshaw Index, Van Hess Index, Perianal Disease Activity Index (PDAI), Rachmilewitz score, Mayo score, Powell-Tuck index, Patient Simple Clinical Colitis Activity Index (P-SCCAI), Lichtiger index, Seo index, Inflammatory Bowel Disease Questionnaire (IBDQ), Manitoba IBD Index, Crohn's Disease Endoscopic Index of Severity (CDEIS), Simple Endoscopic Score for Crohn Disease (SES-CD), Lewis score (capsule endoscopy), Rutgeert's Score, and the Montreal Classification, and IBD questionnaire. In some instances, the subject is not diagnosed with the disease or condition. In some instances, the subject is suffering from a symptom related to a disease or condition disclosed herein (e.g., abdominal pain, cramping, diarrhea, rectal bleeding, fever, weight loss, fatigue, loss of appetite, dehydration, and malnutrition, anemia, or ulcers).

In some embodiments, the subject is susceptible to, or is inflicted with, thiopurine toxicity, or a disease caused by thiopurine toxicity (such as pancreatitis or leukopenia). In further embodiments provided, the subject is, or is suspected of being, non-responsive to a standard treatment (e.g., anti-TNF alpha therapy, anti-a4-b7 therapy (vedolizumab), anti-IL12p40 therapy (ustekinumab), Thalidomide, or Cytoxin). In some cases, the subject is not responsive to the induction of said therapy. In some cases, the subject loses response to said standard treatment after a period of time during treatment.

Interleukin 18 Receptor 1 (IL18R1)

Interleukin 18 Receptor 1 (IL18R1) (UniProtKB: Q13478) is encoded by the gene IL18R1 (Entrez Gene: 8809), which is a cytokine receptor that belongs to the interleukin 1 receptor family. This receptor specifically binds interleukin 18 (IL18), and is essential for IL18 mediated signal transduction. IFN-alpha and IL12 are reported to induce the expression of this receptor in NK and T cells. This gene along with four other members of the interleukin 1 receptor family, including IL1R2, IL1R1, ILRL2 (IL-1Rrp2), and IL1RL1 (T1/ST2), forma gene cluster on chromosome 2q.

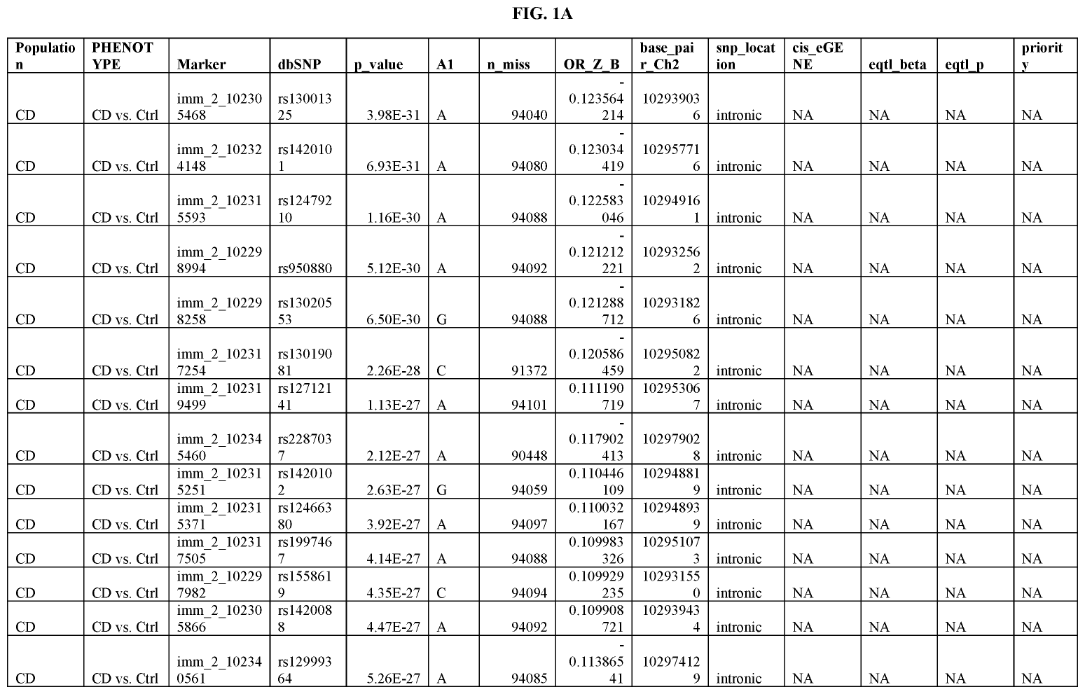

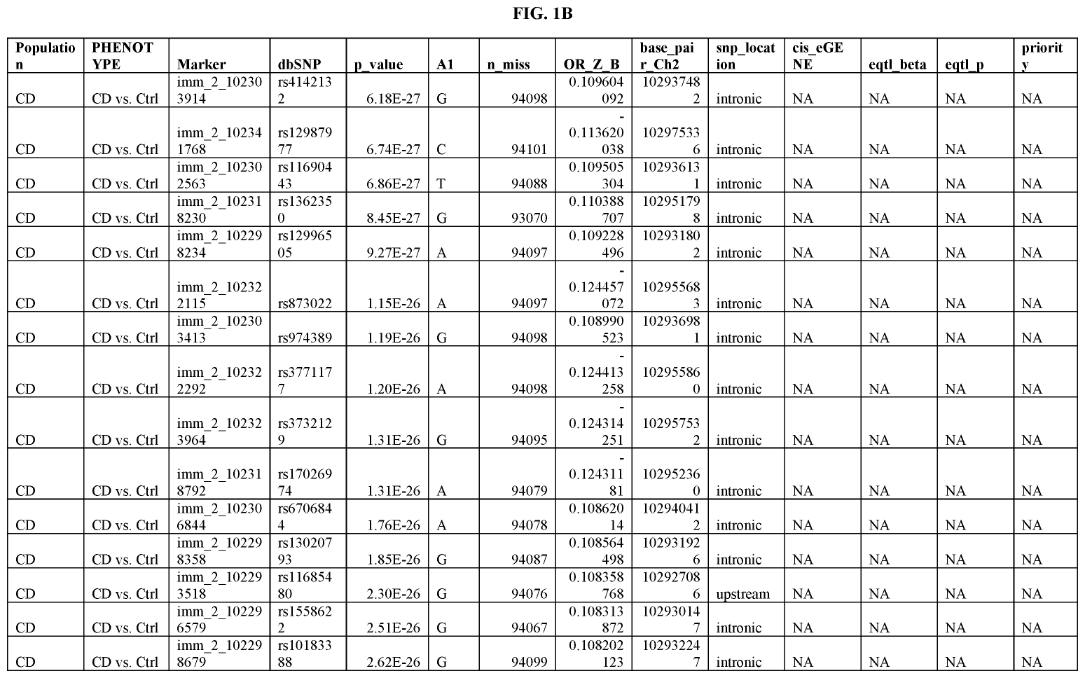

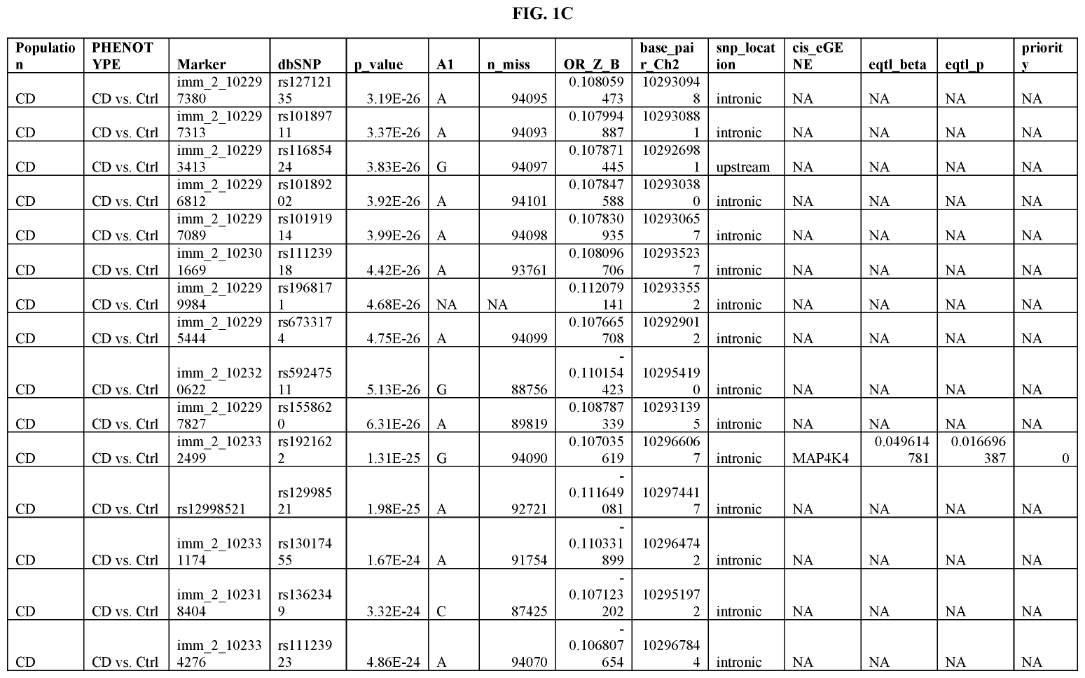

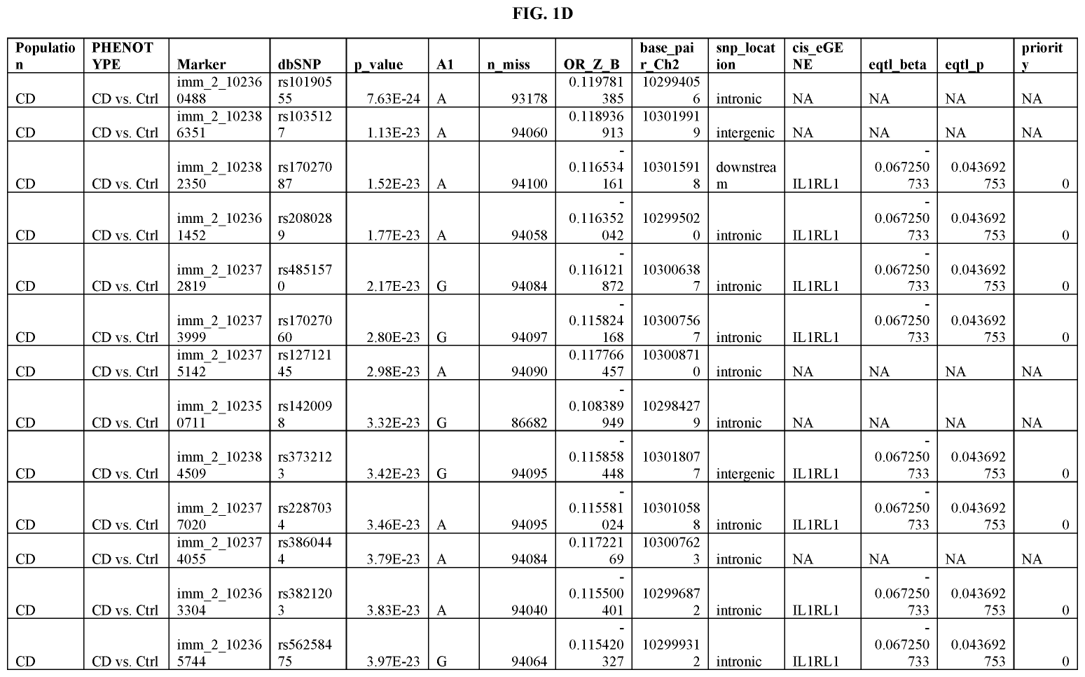

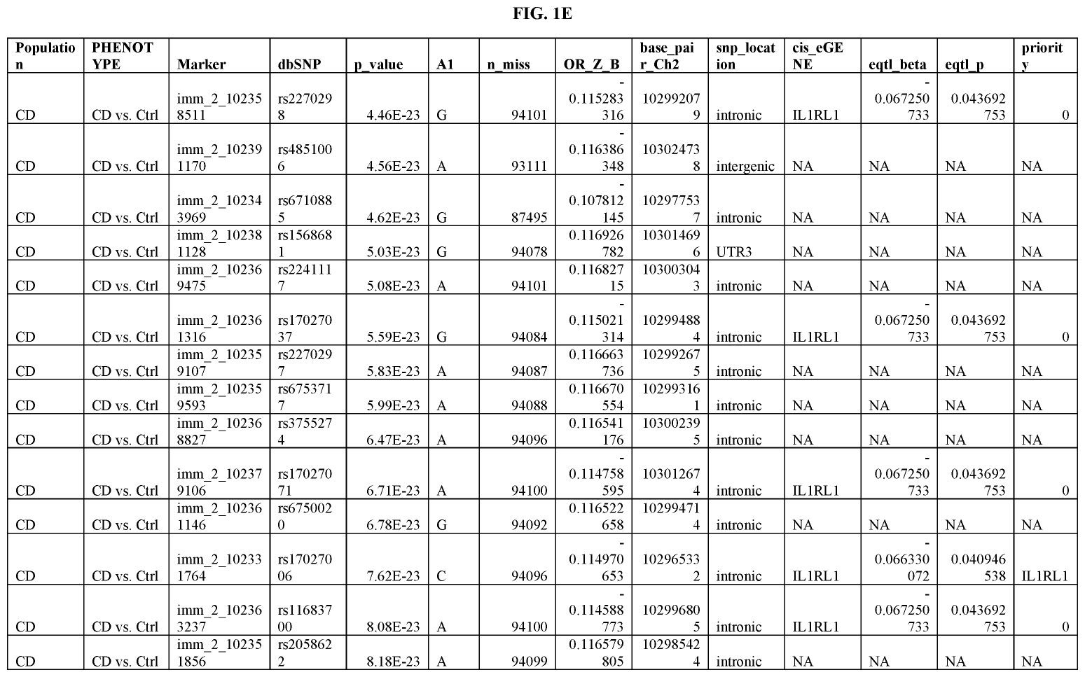

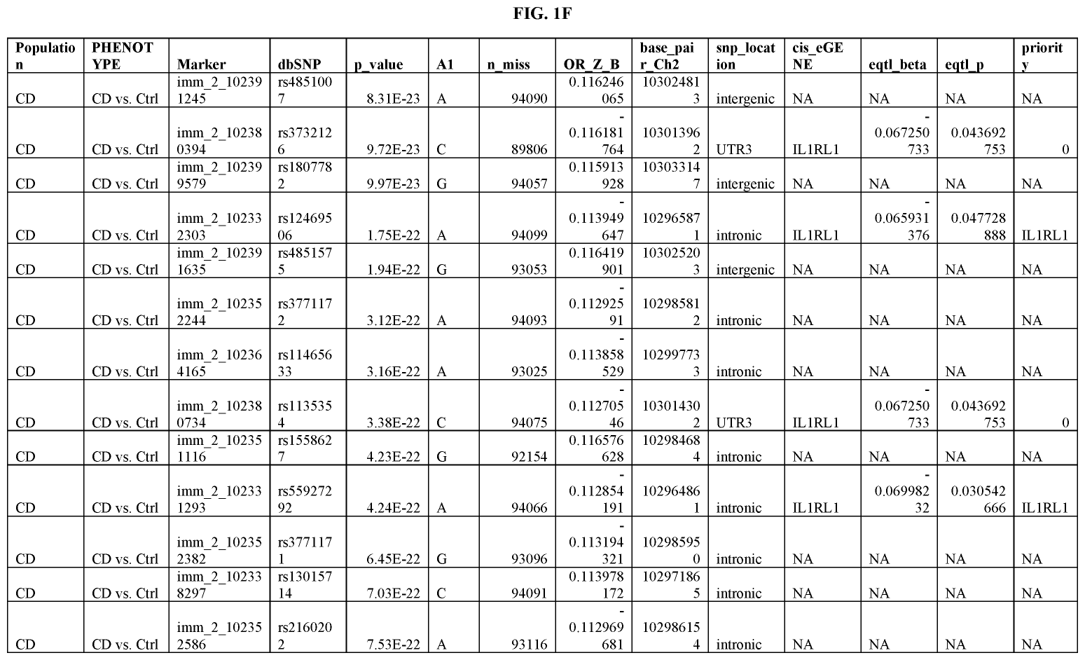

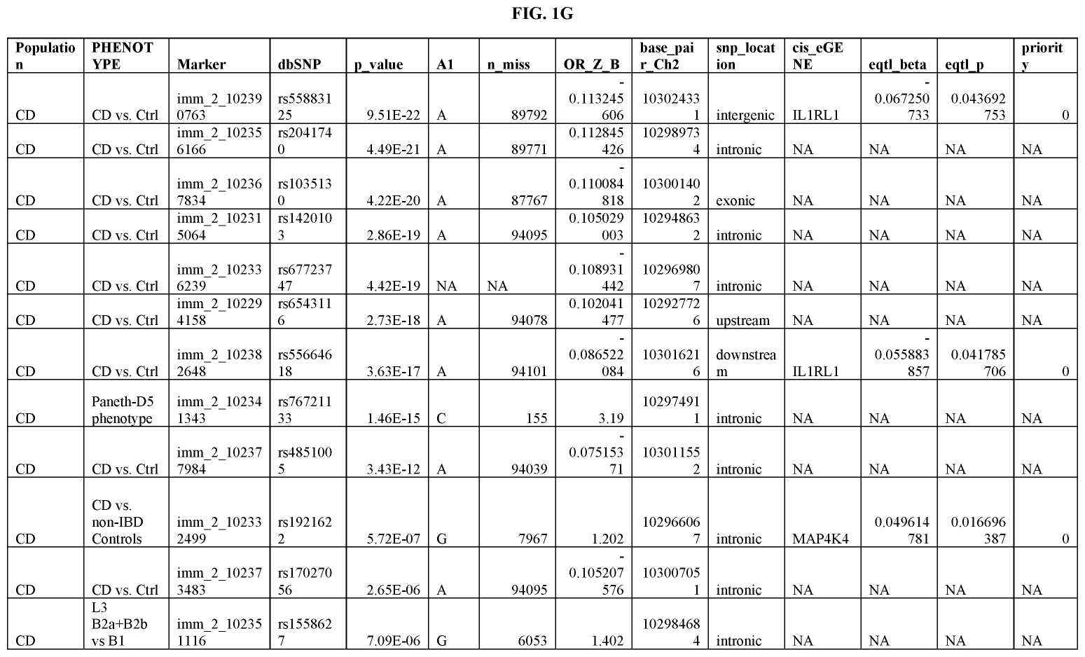

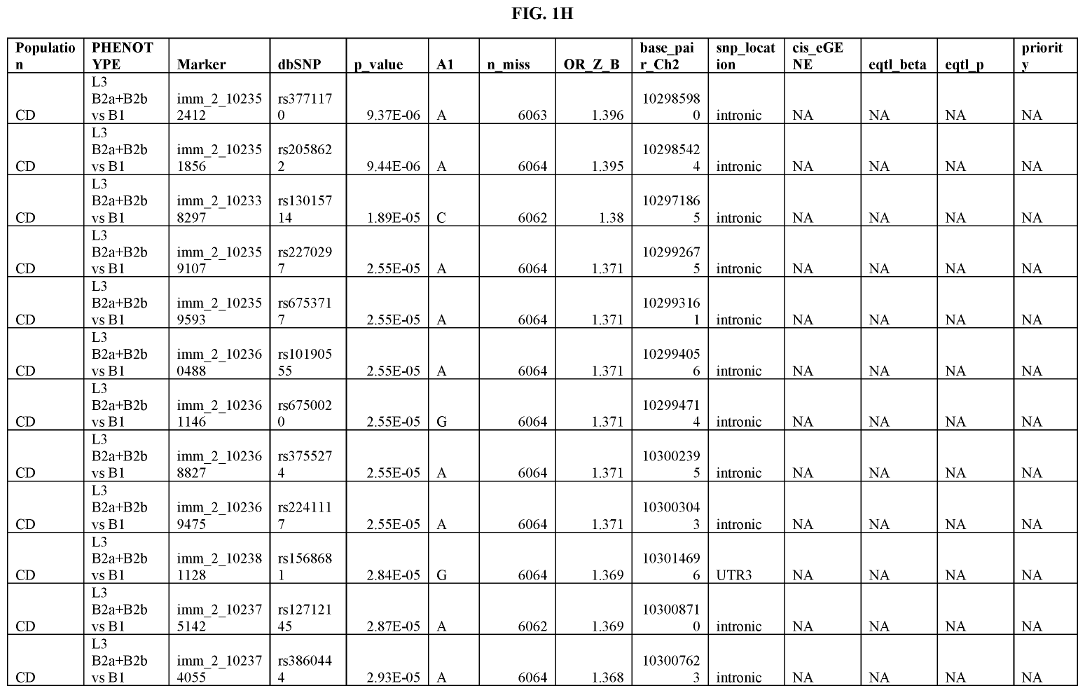

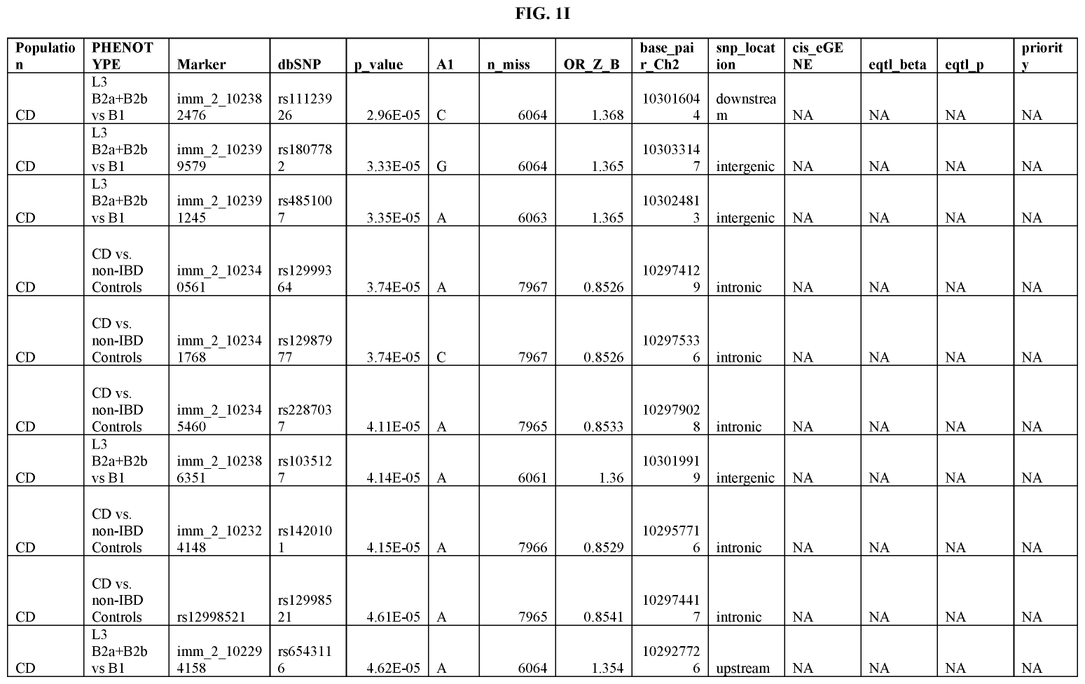

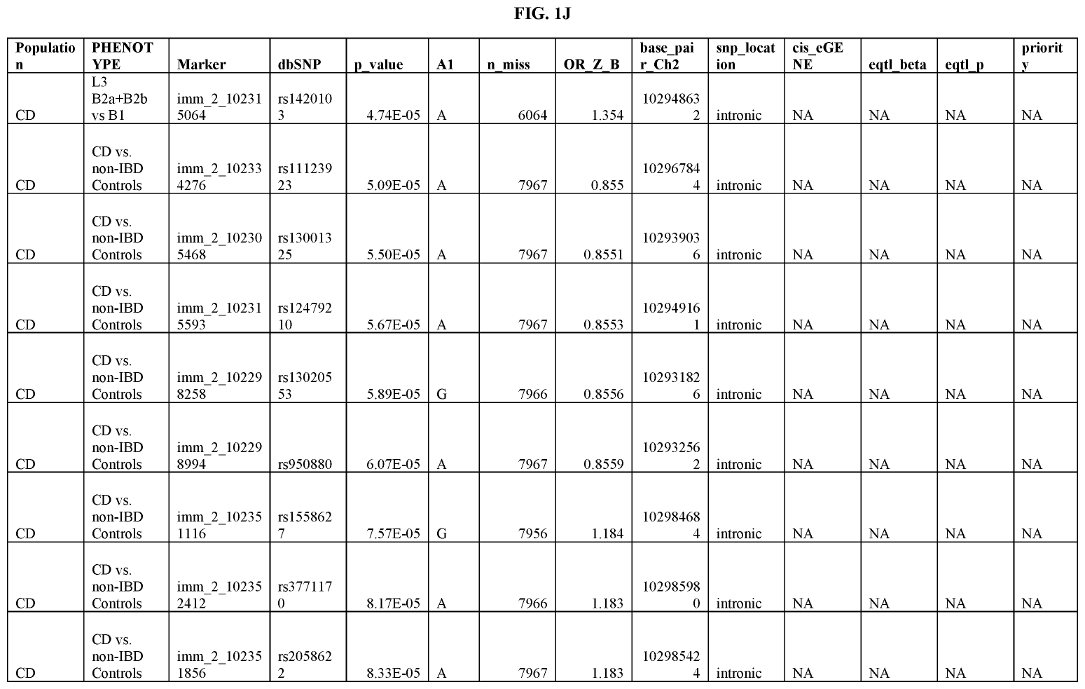

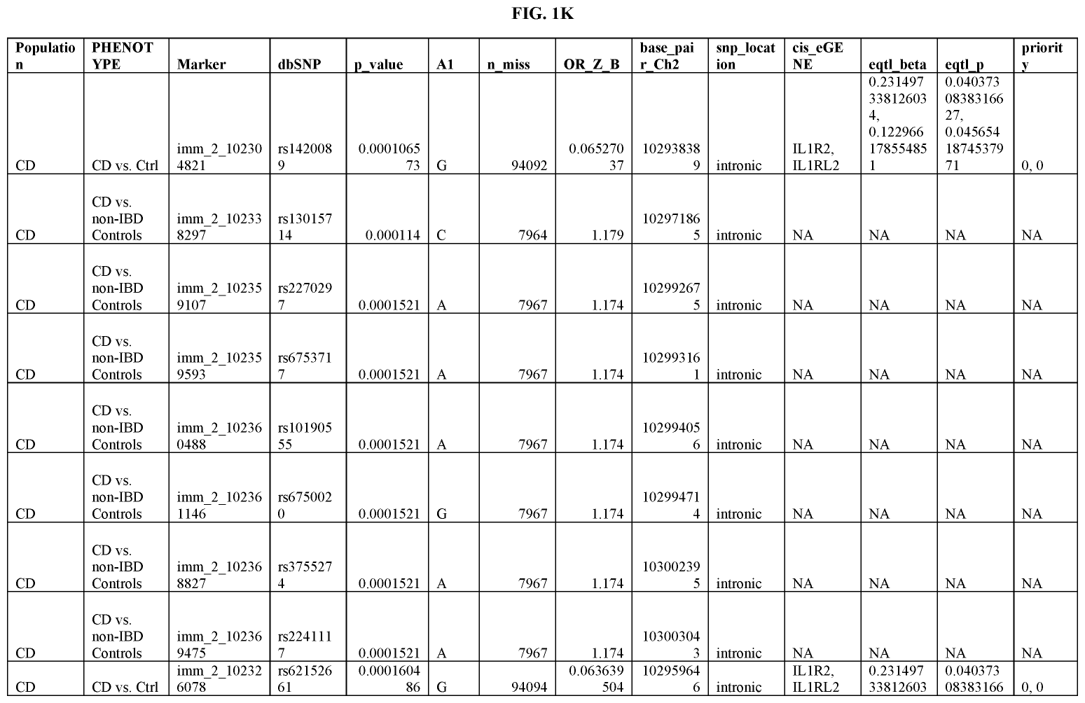

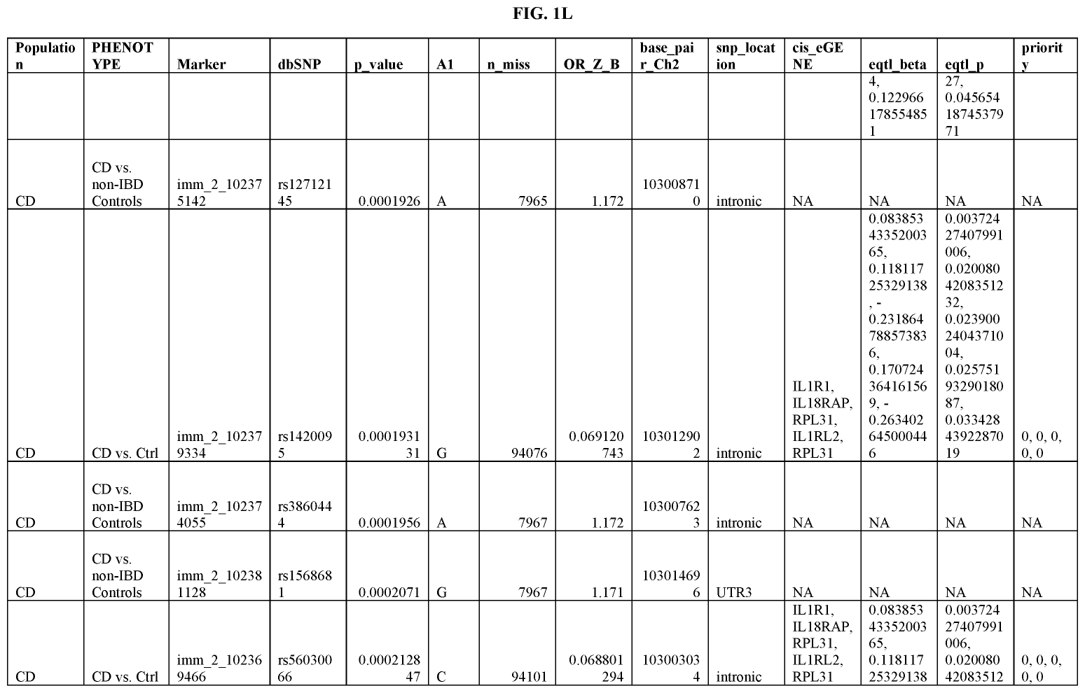

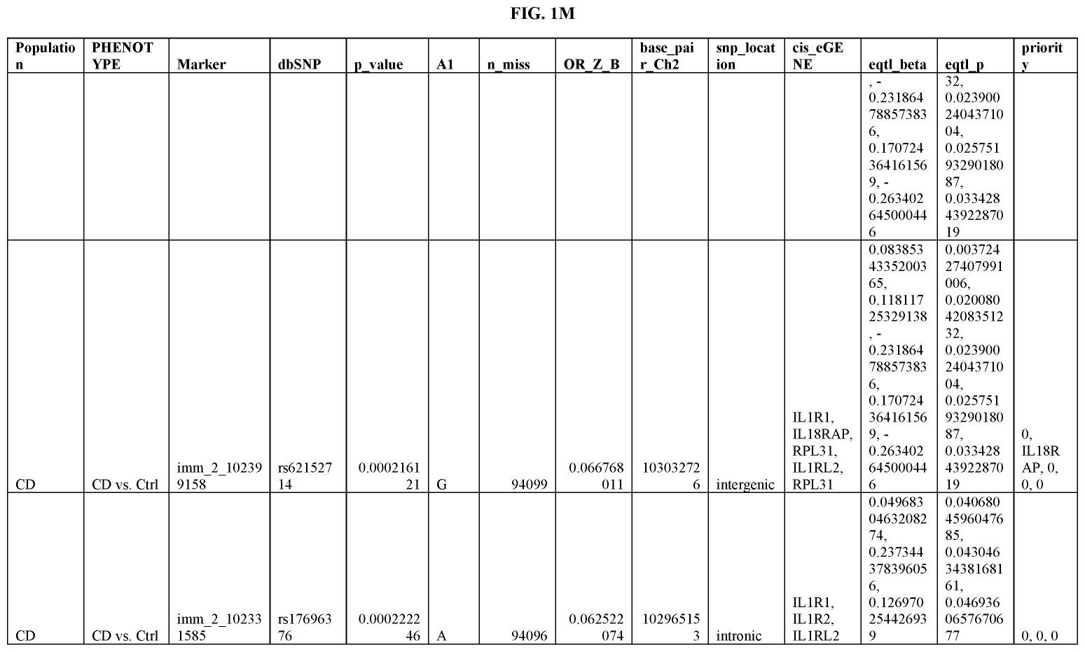

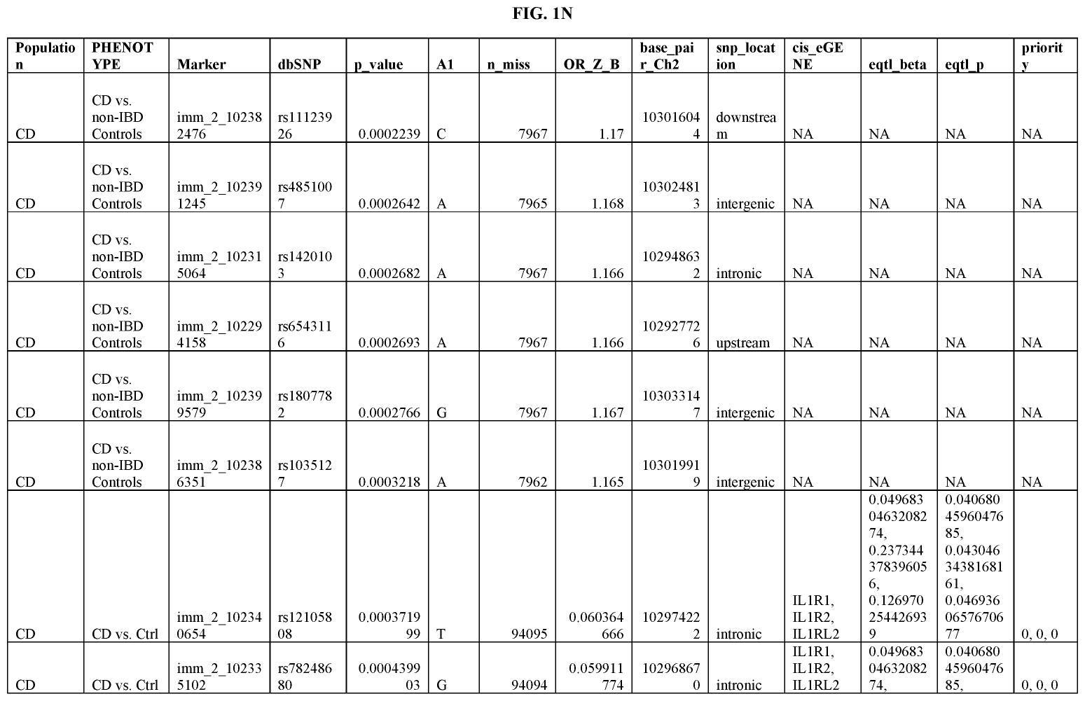

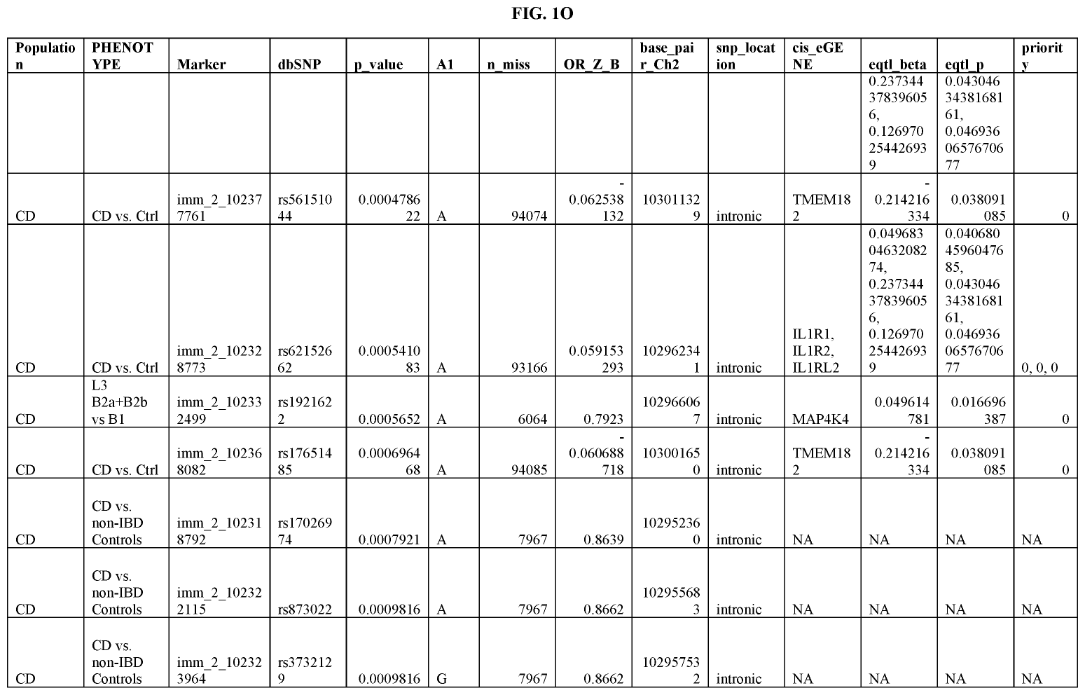

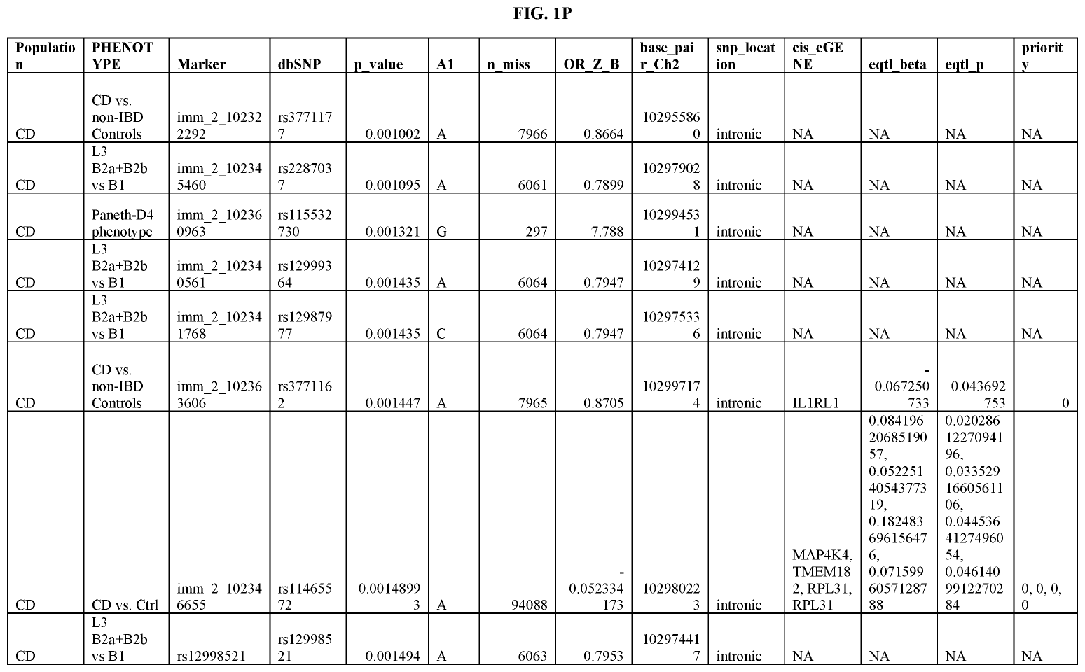

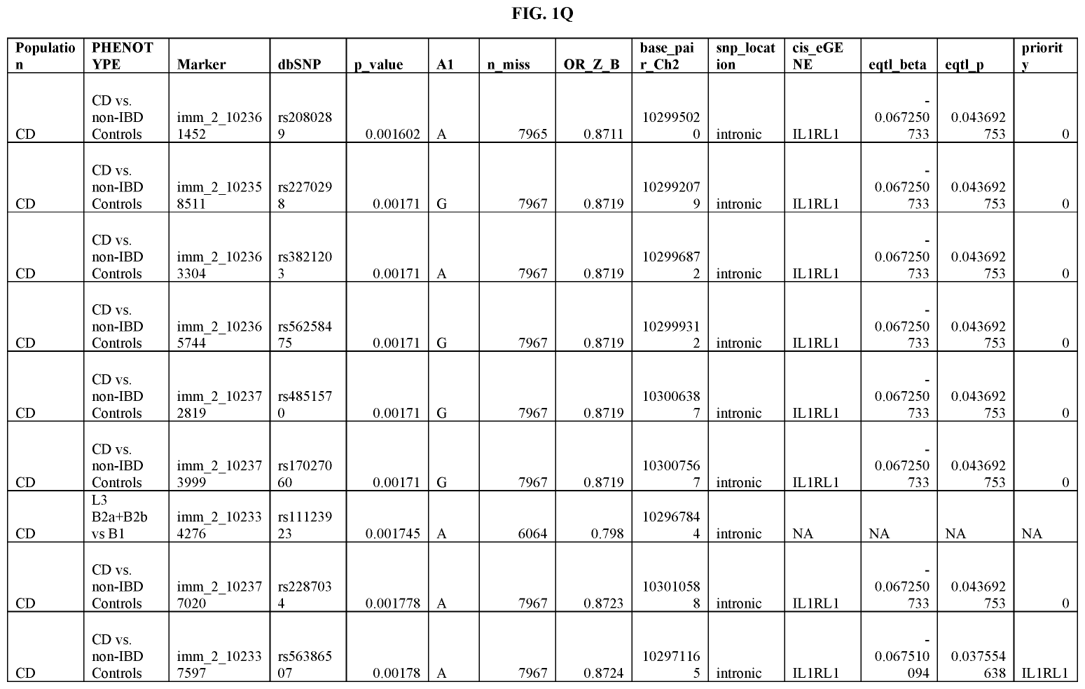

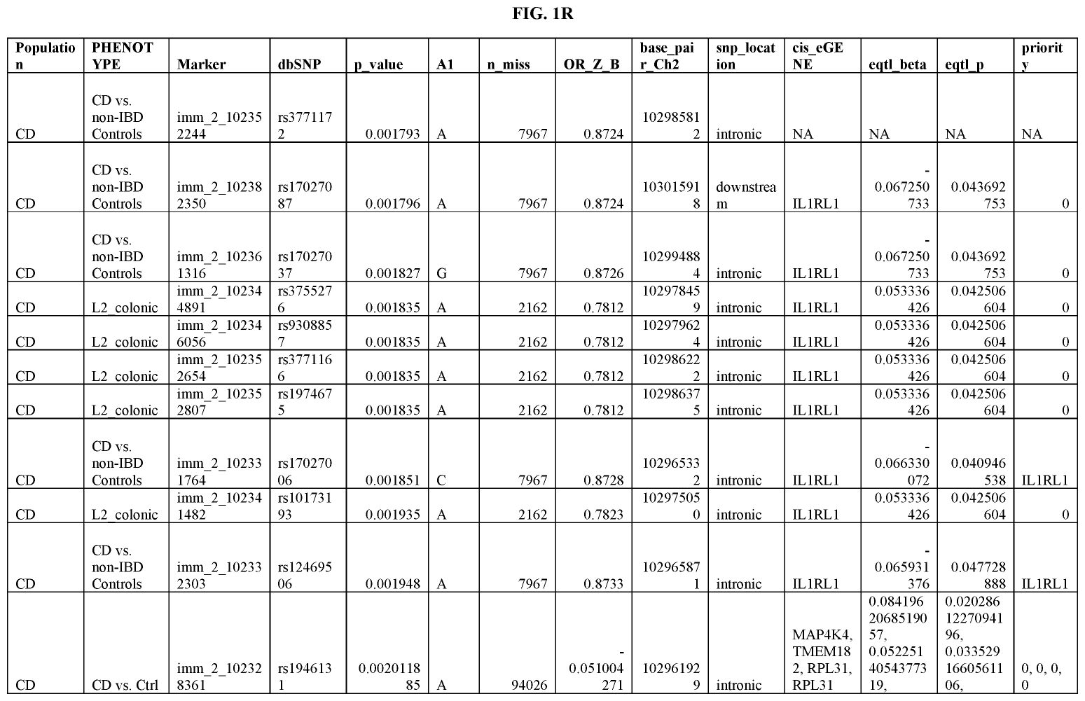

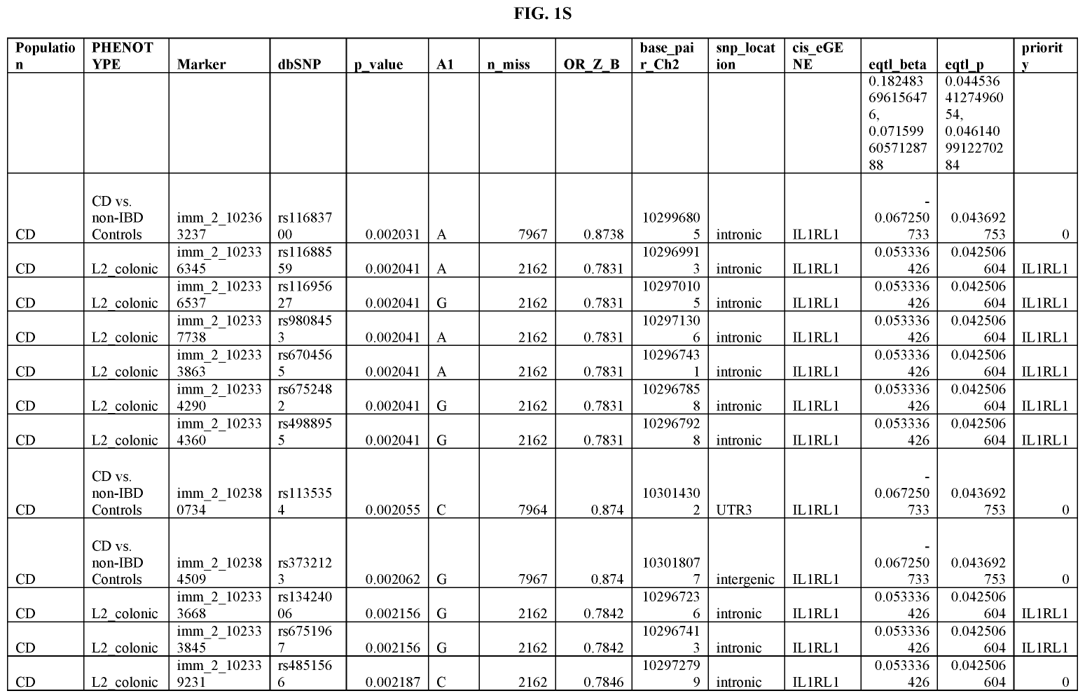

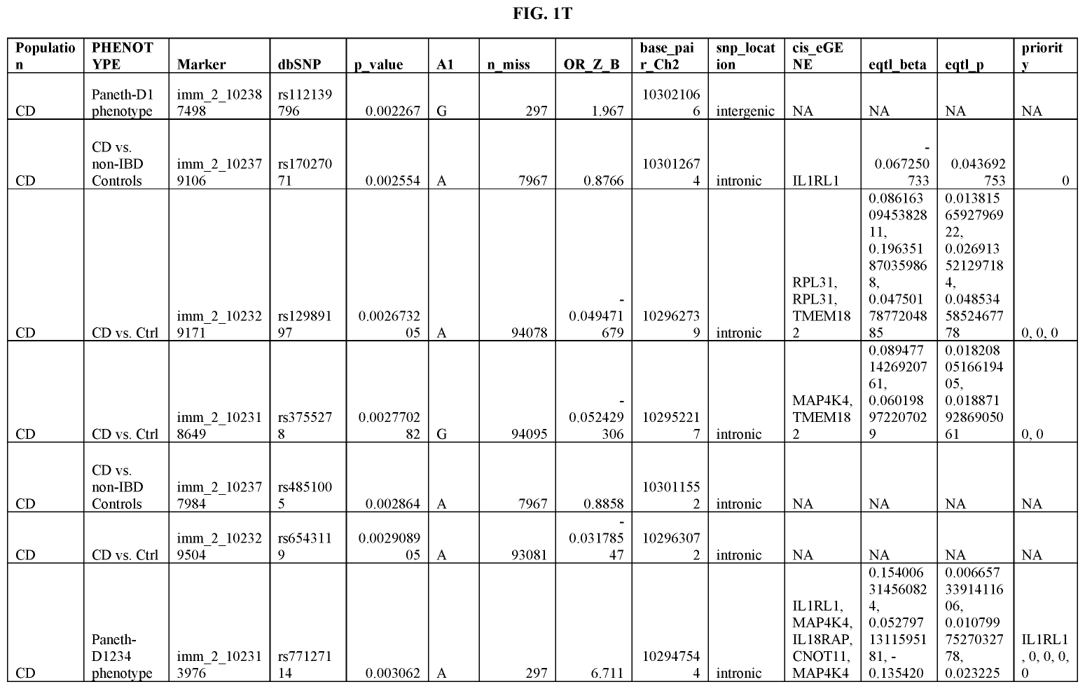

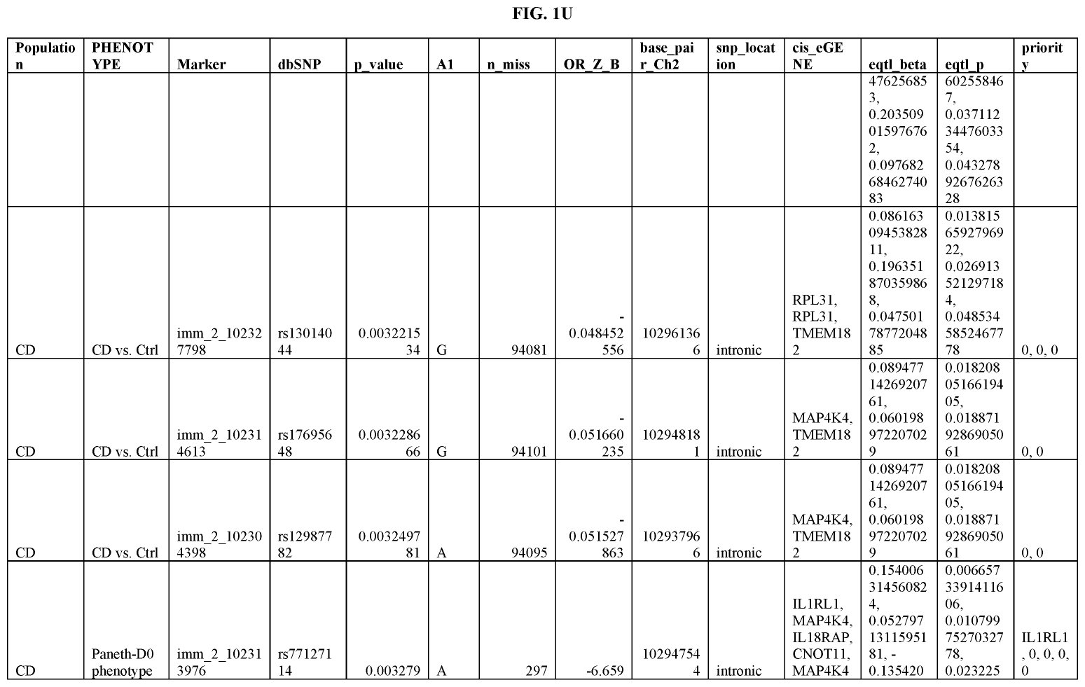

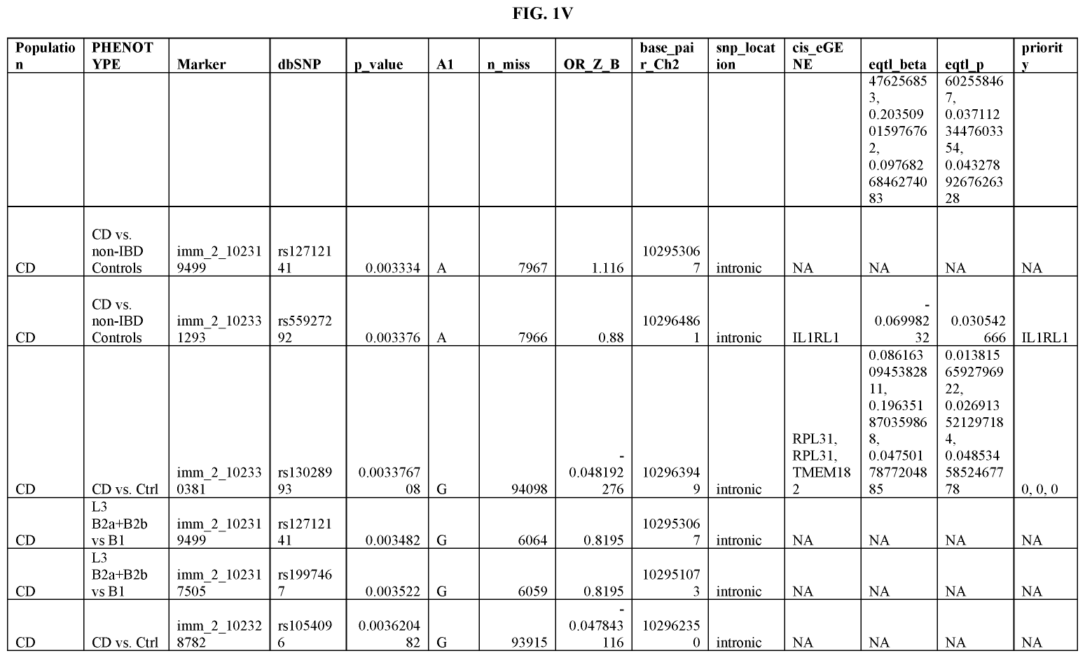

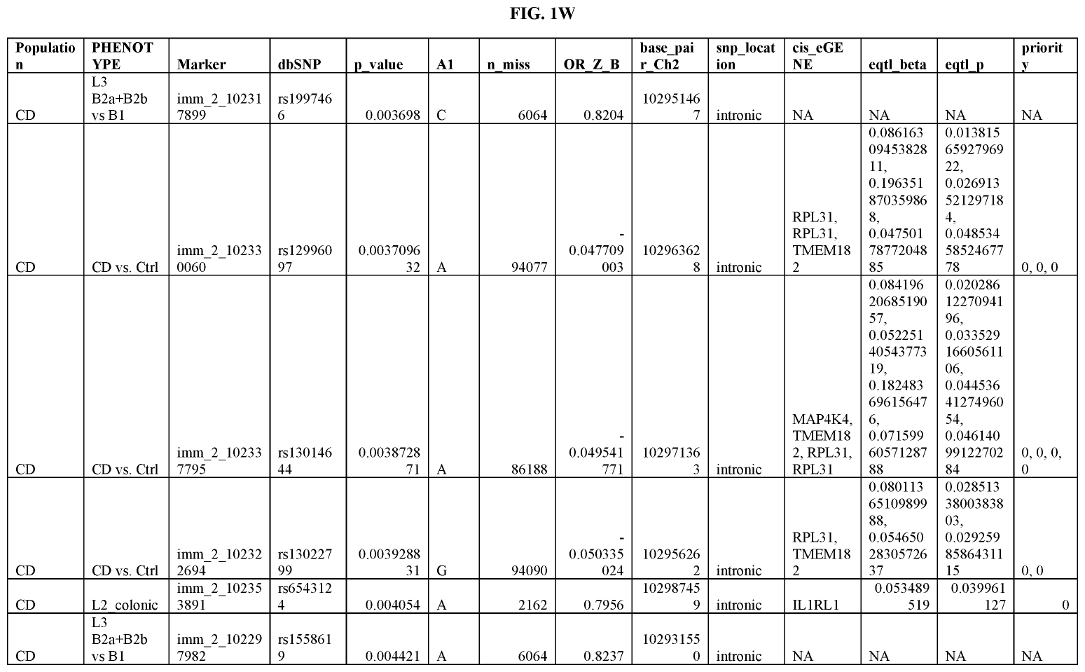

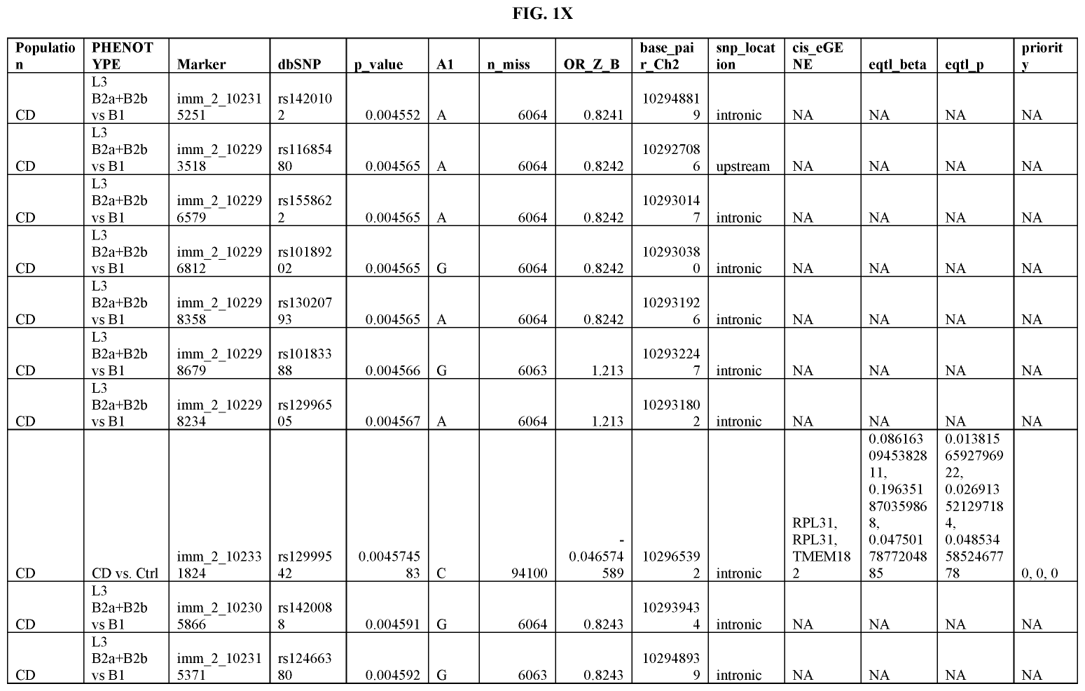

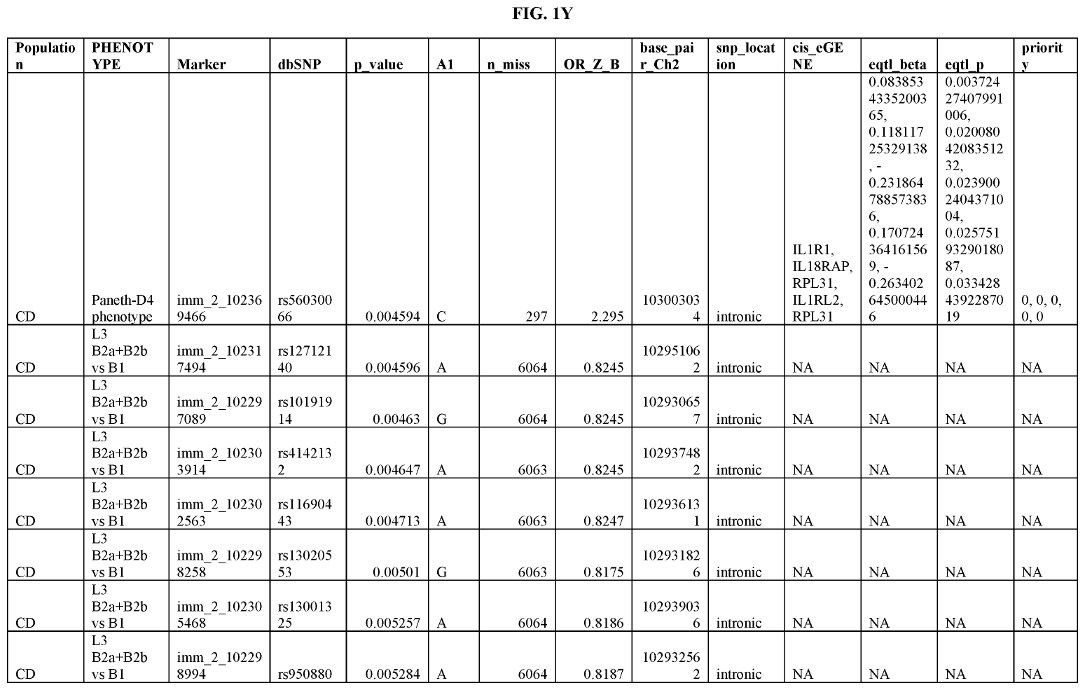

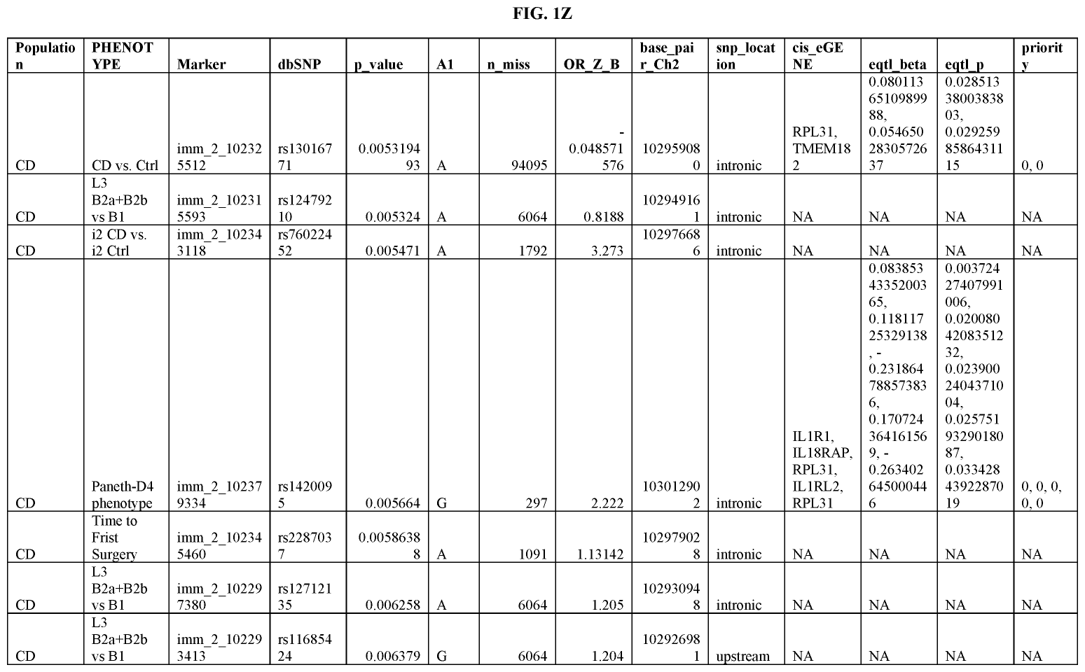

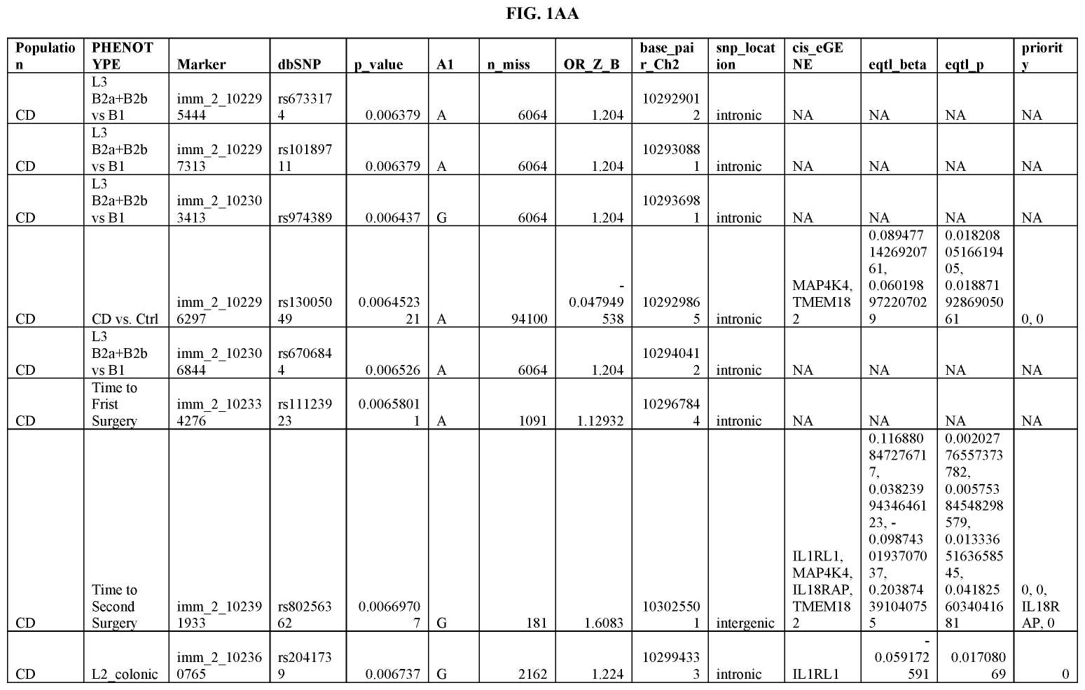

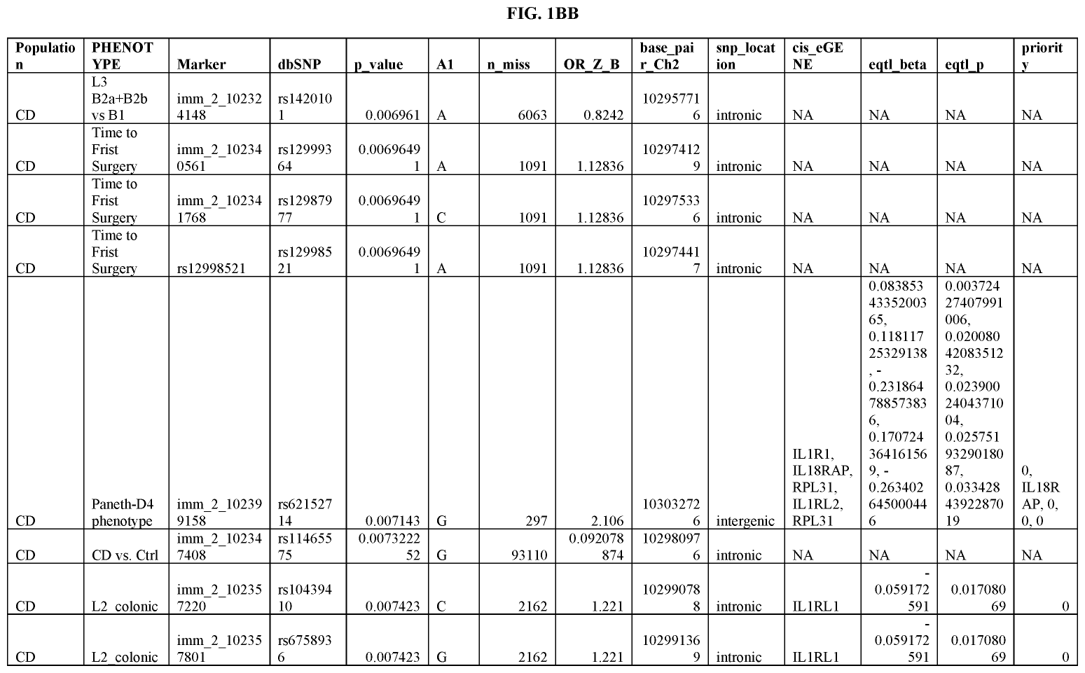

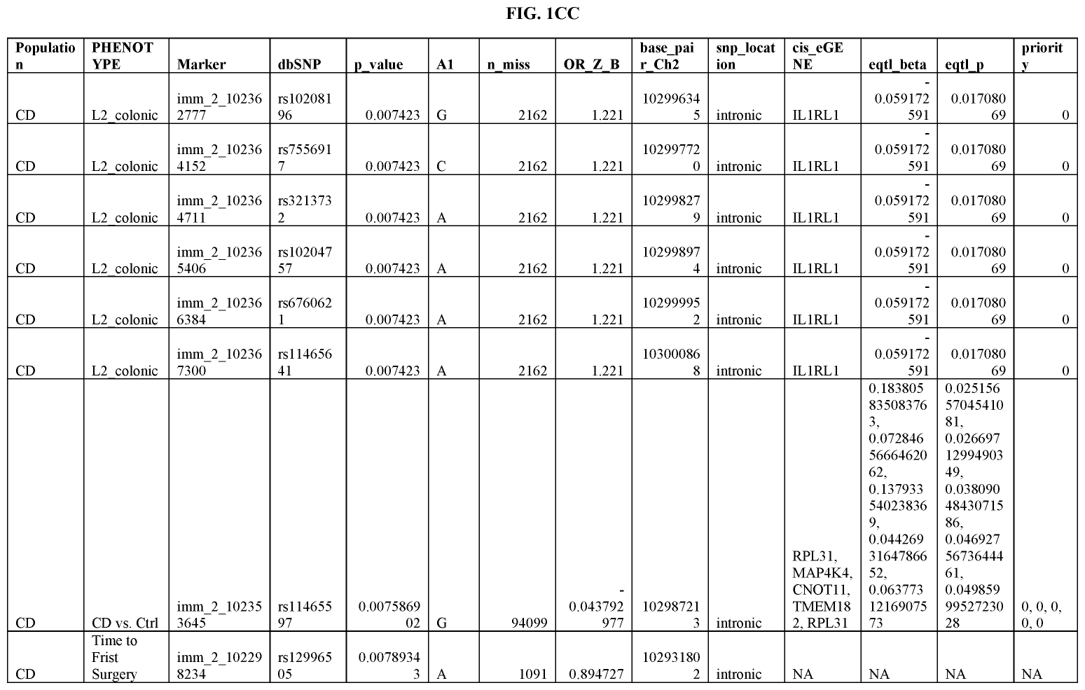

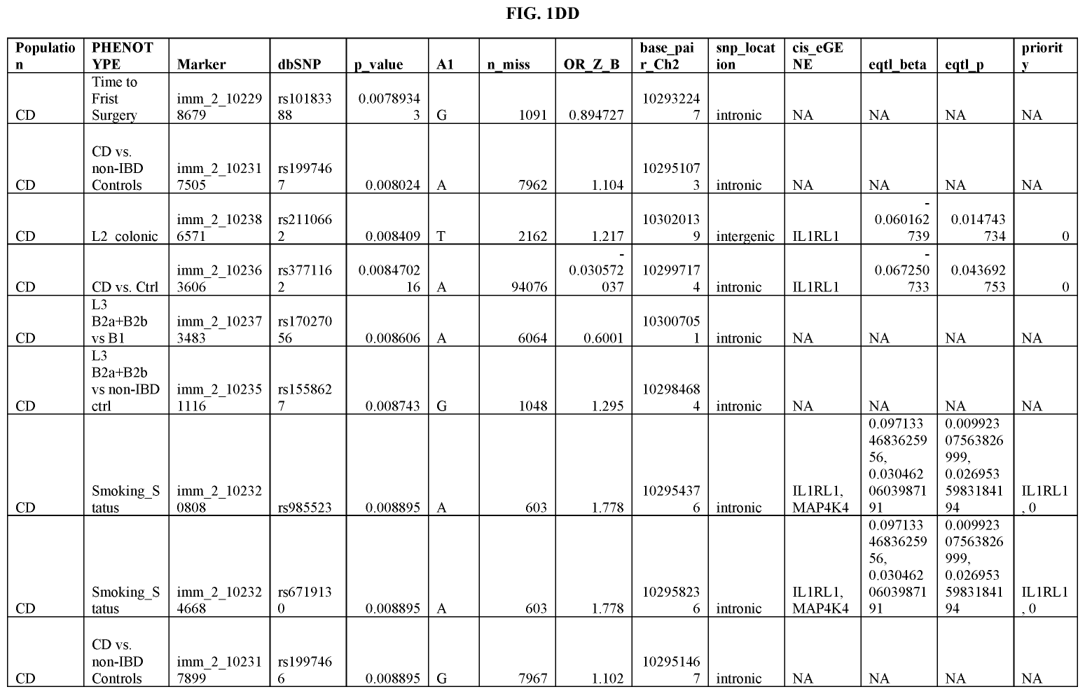

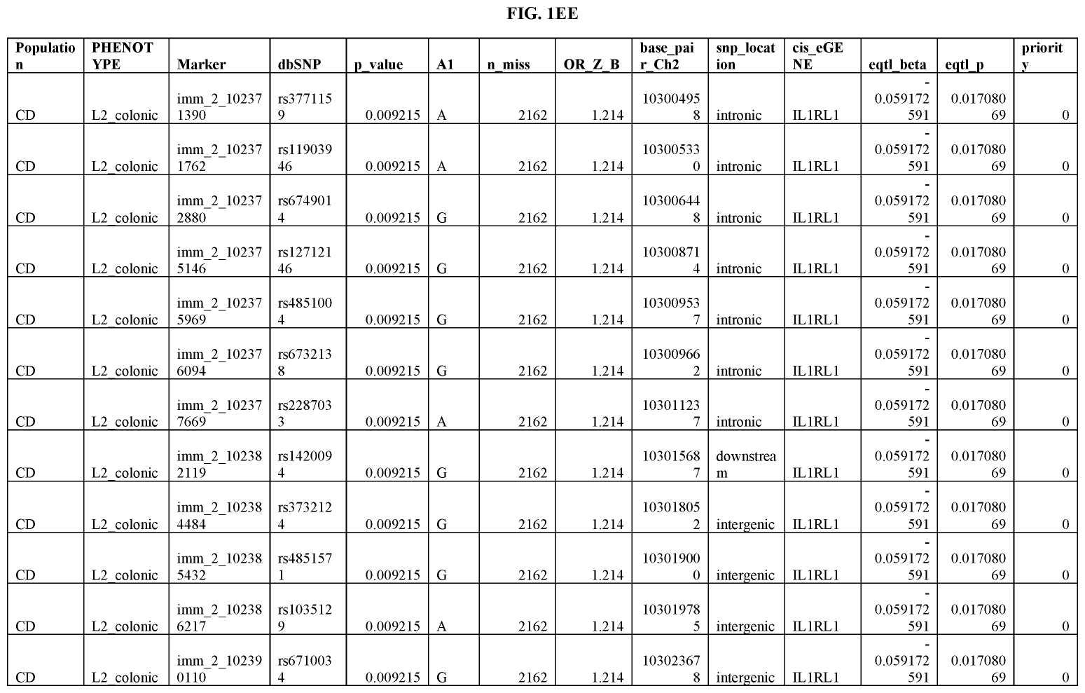

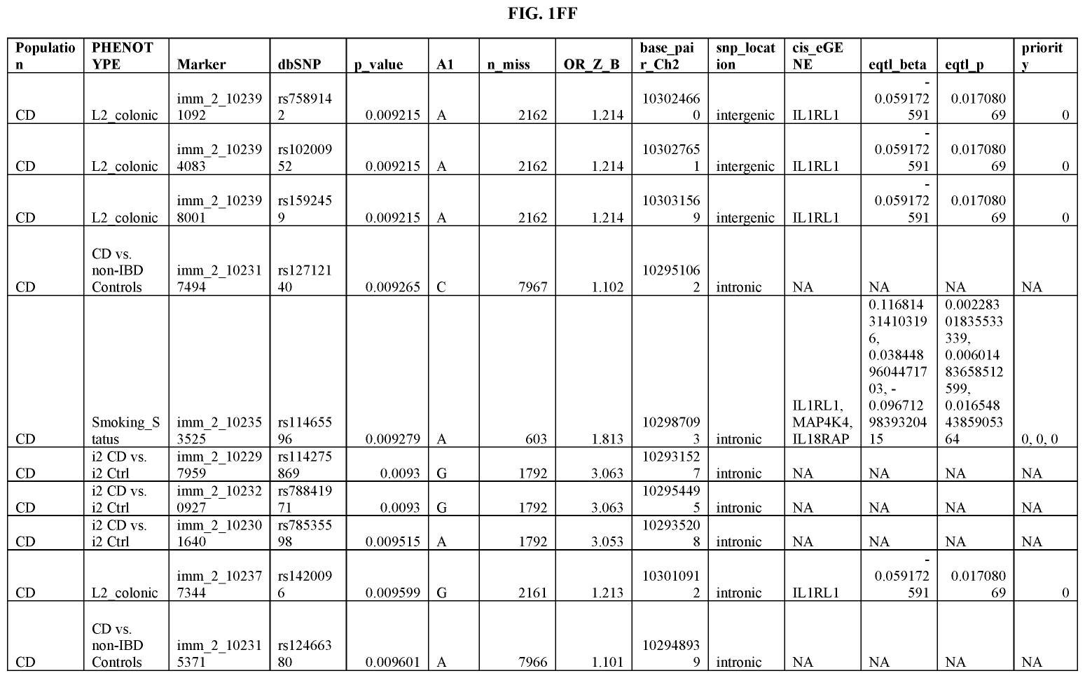

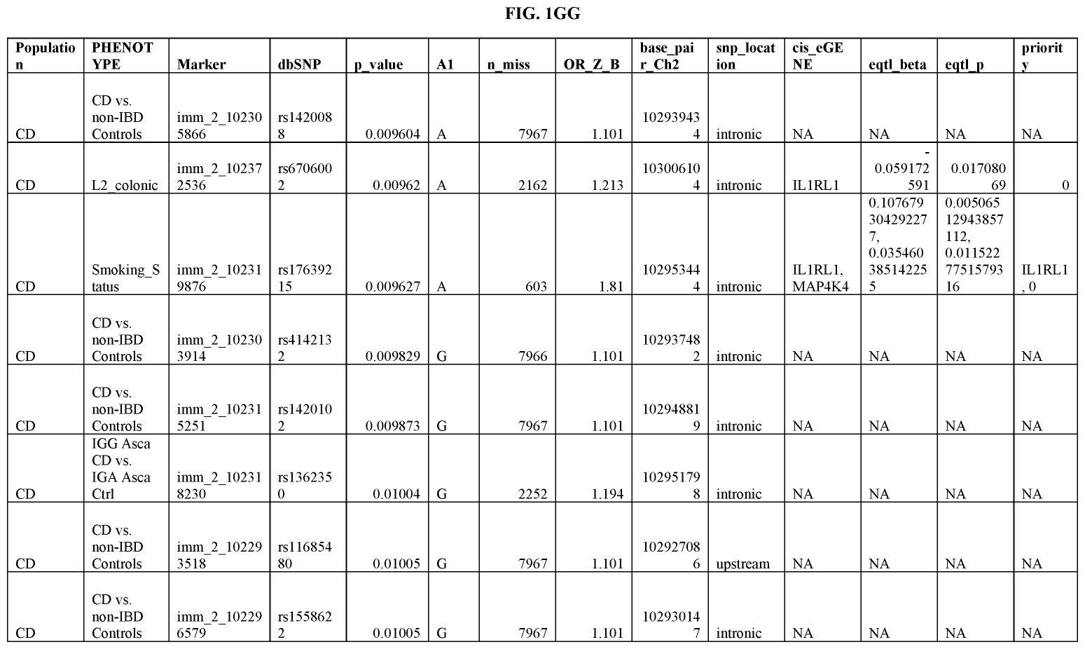

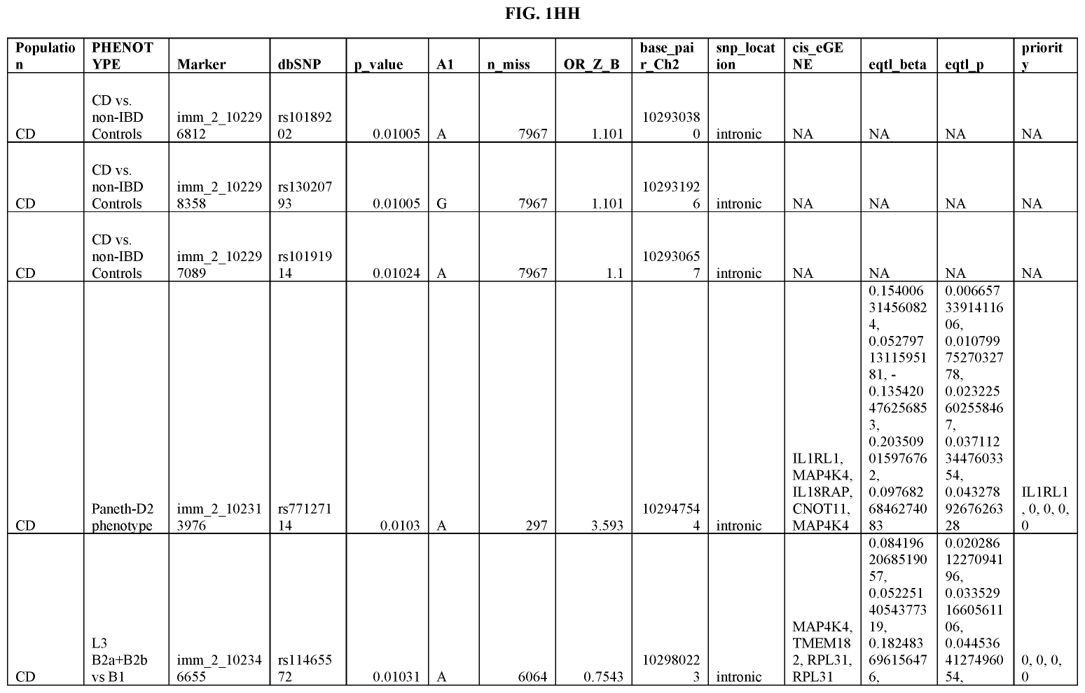

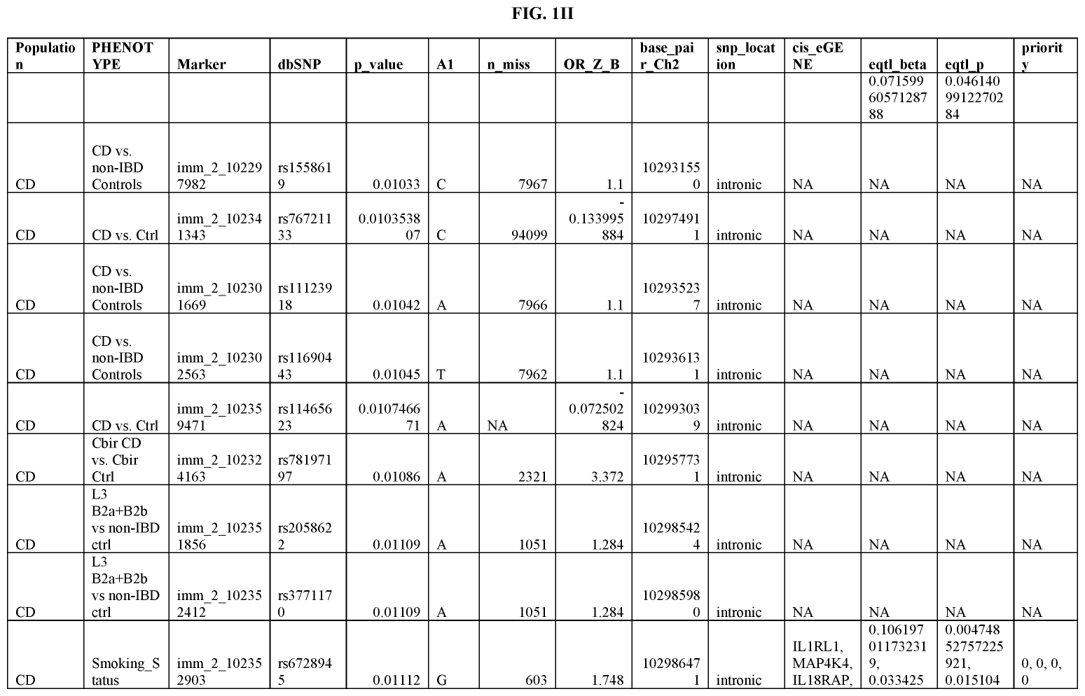

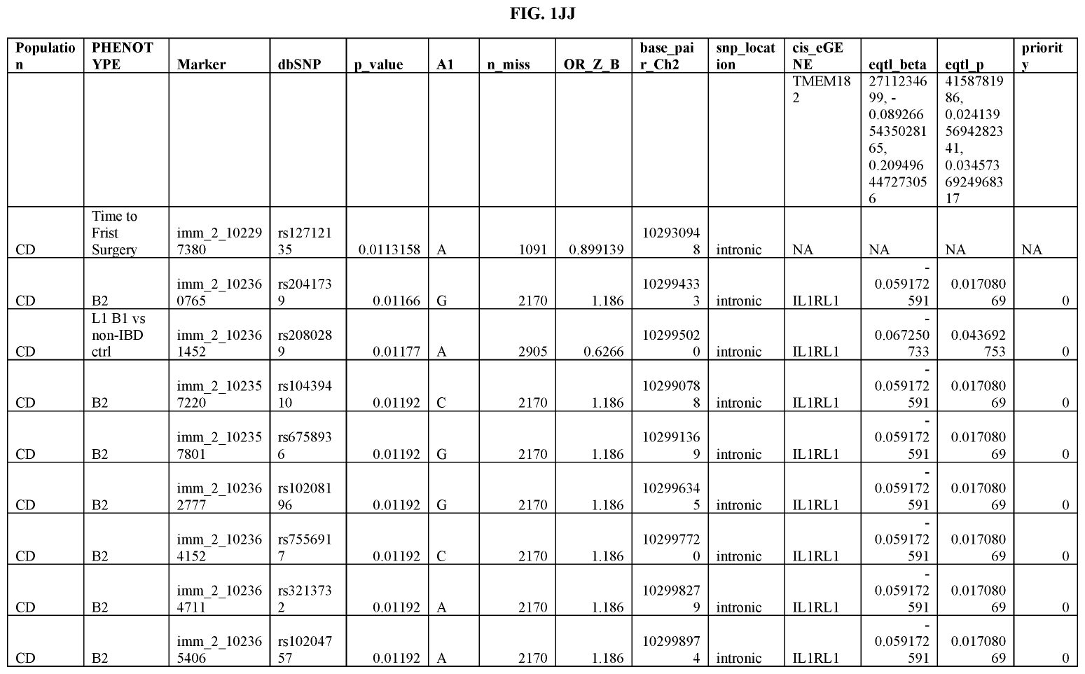

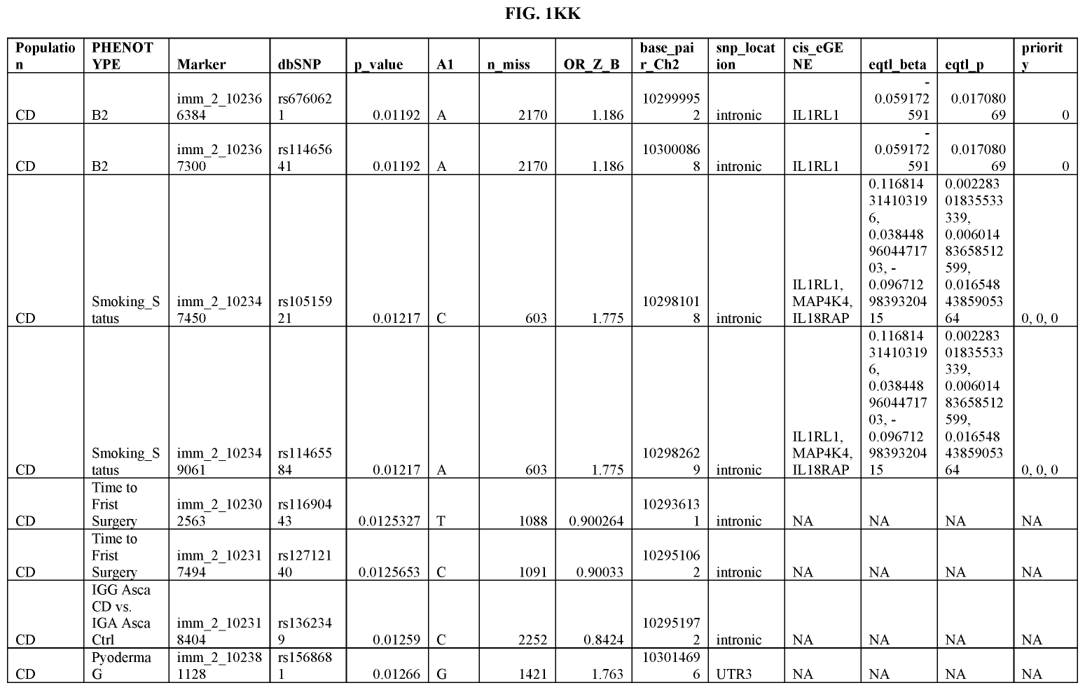

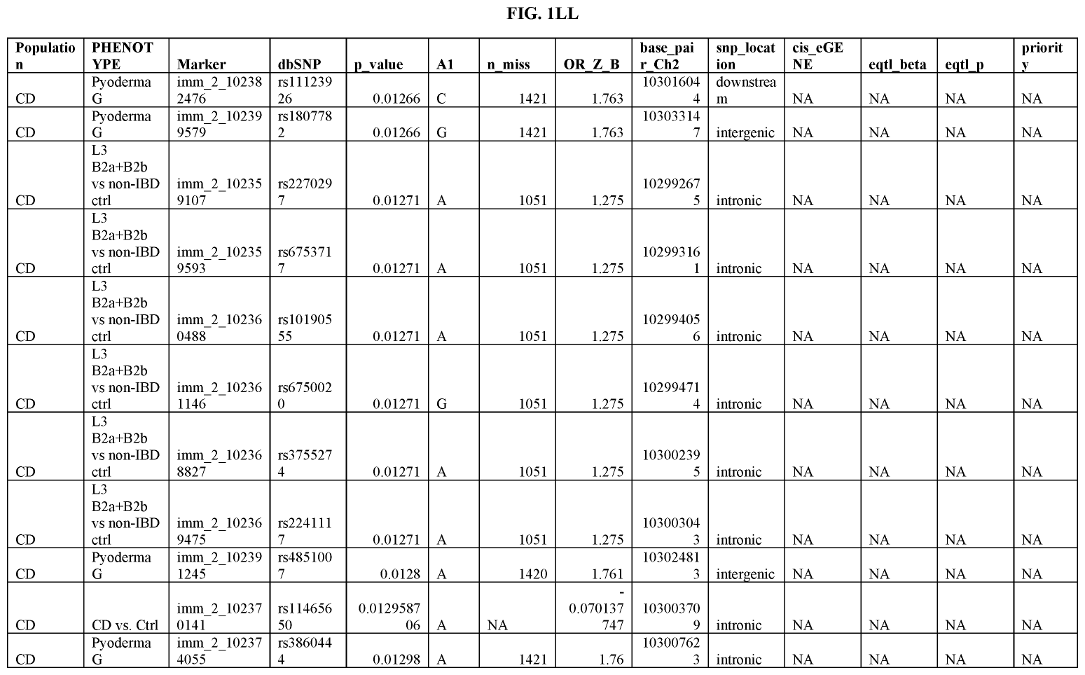

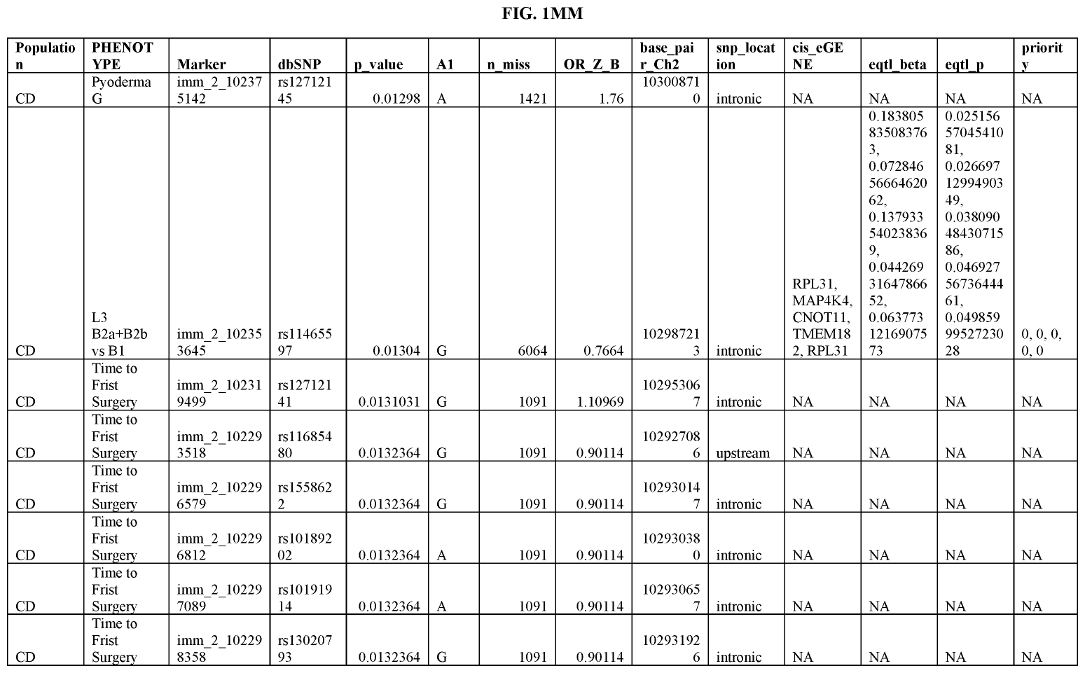

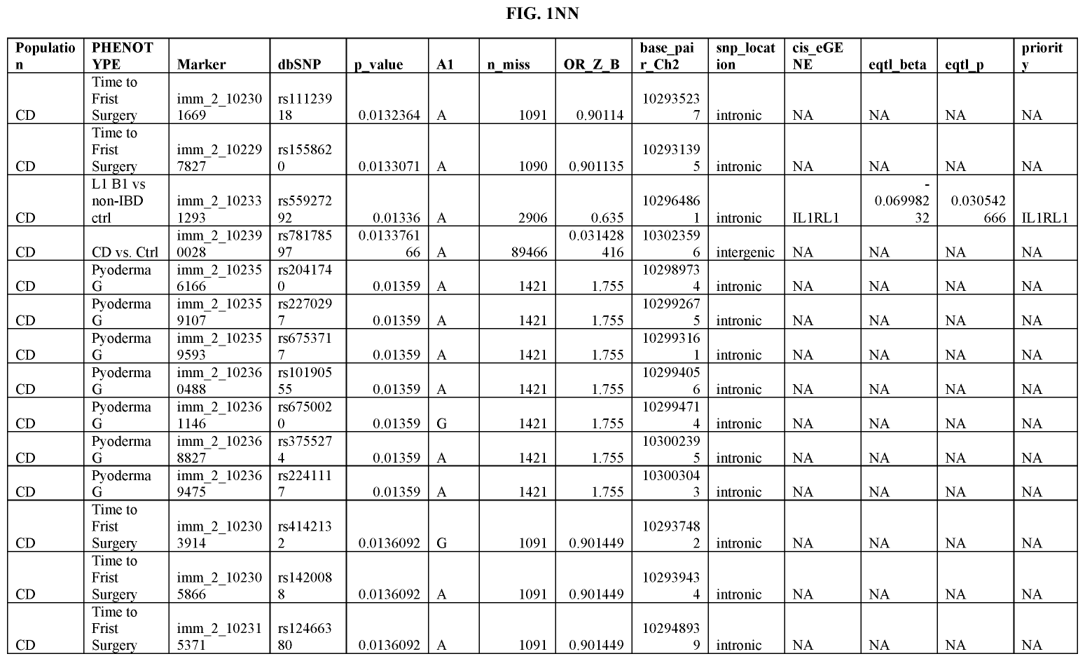

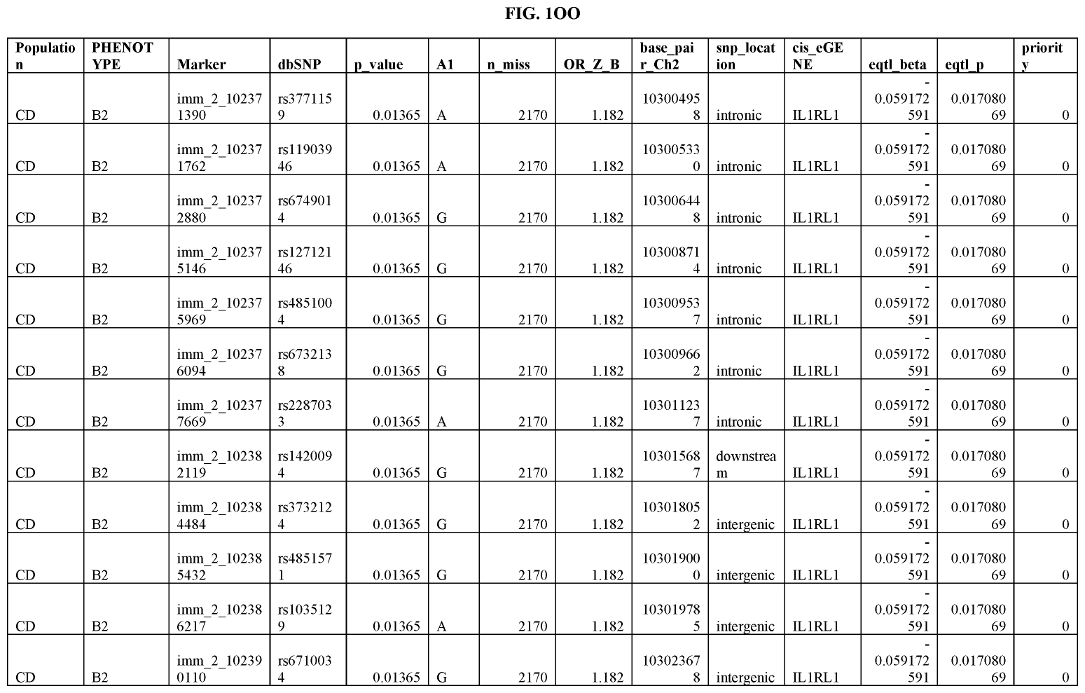

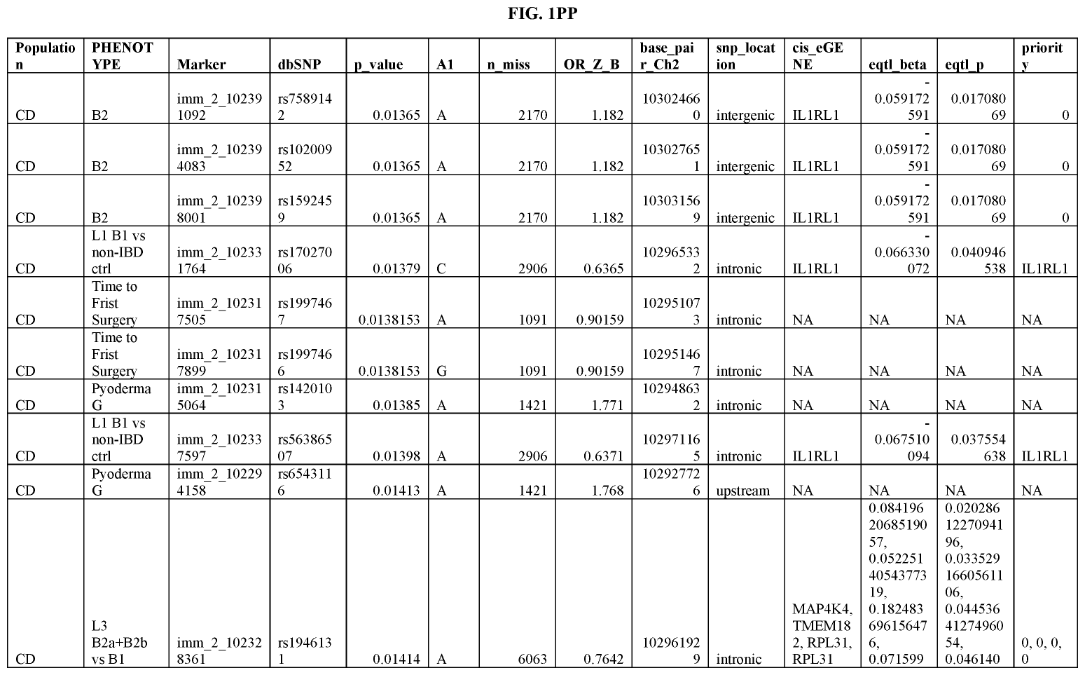

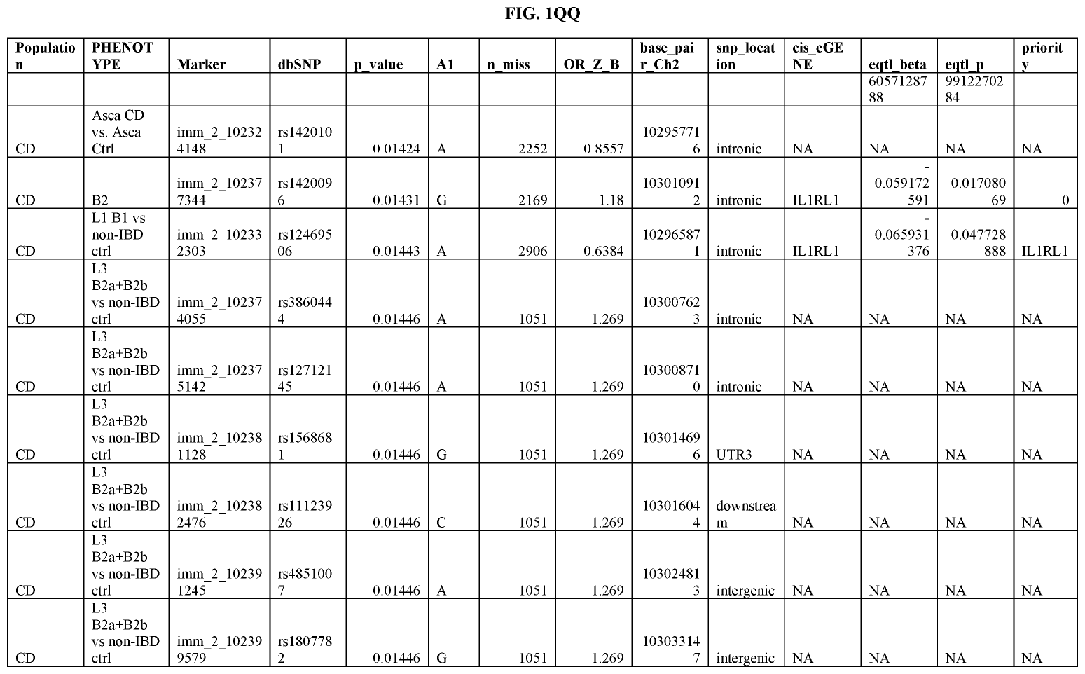

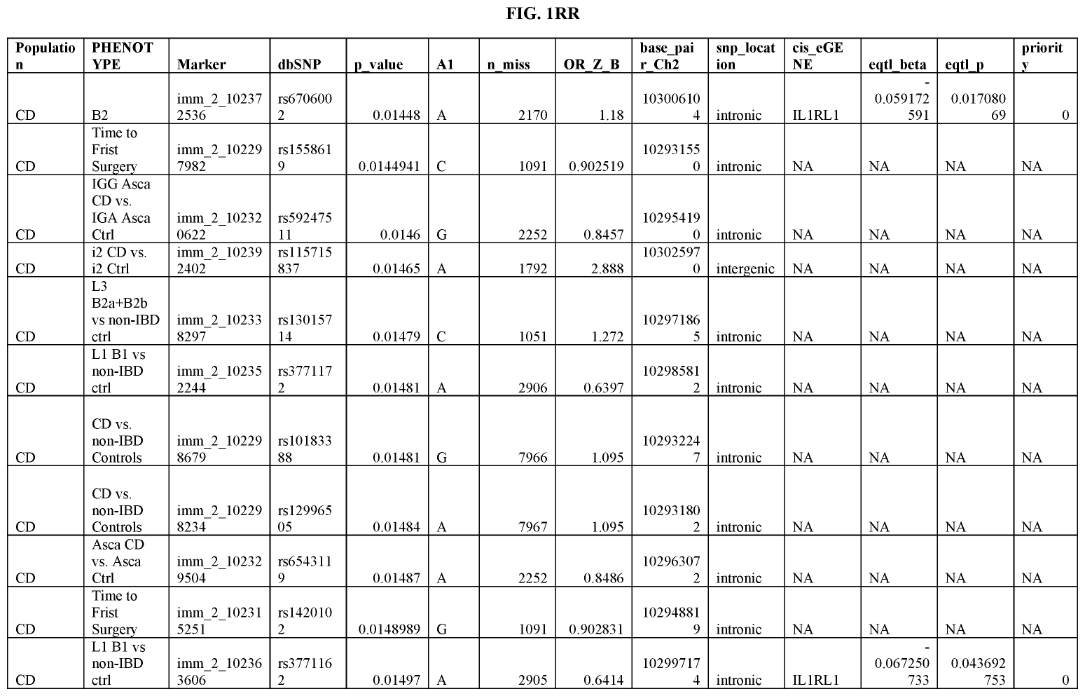

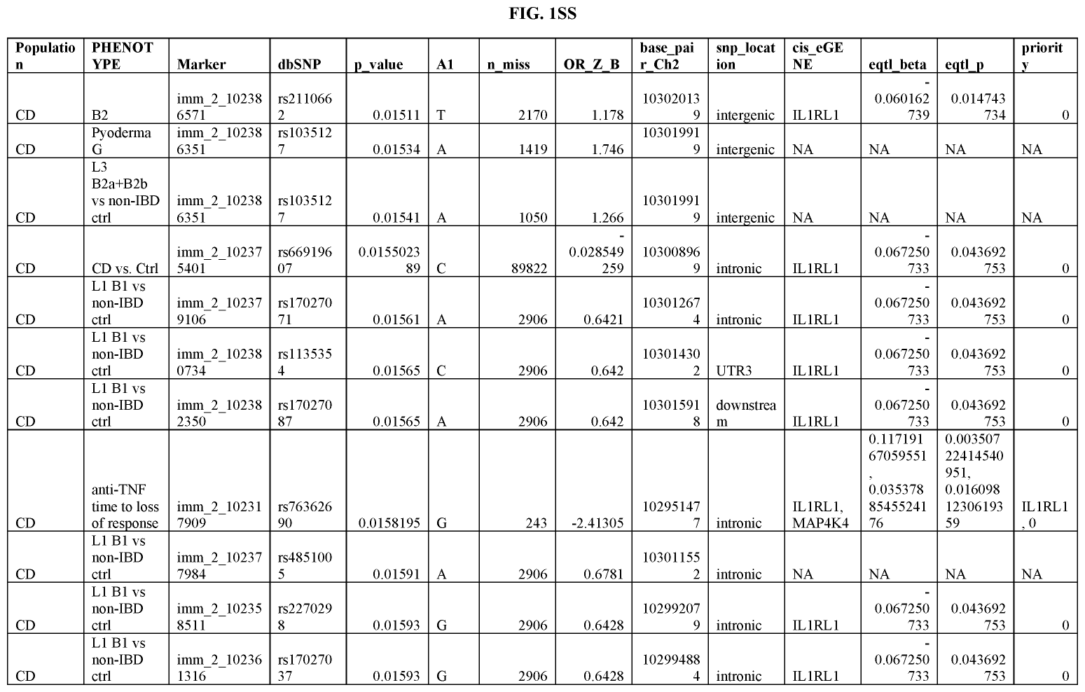

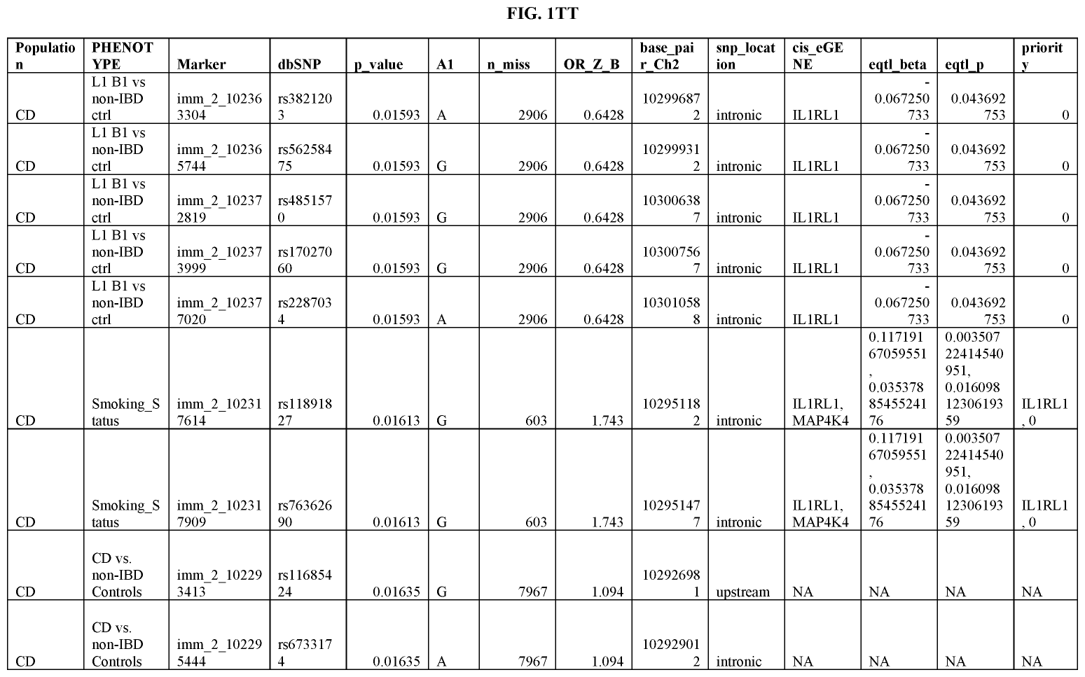

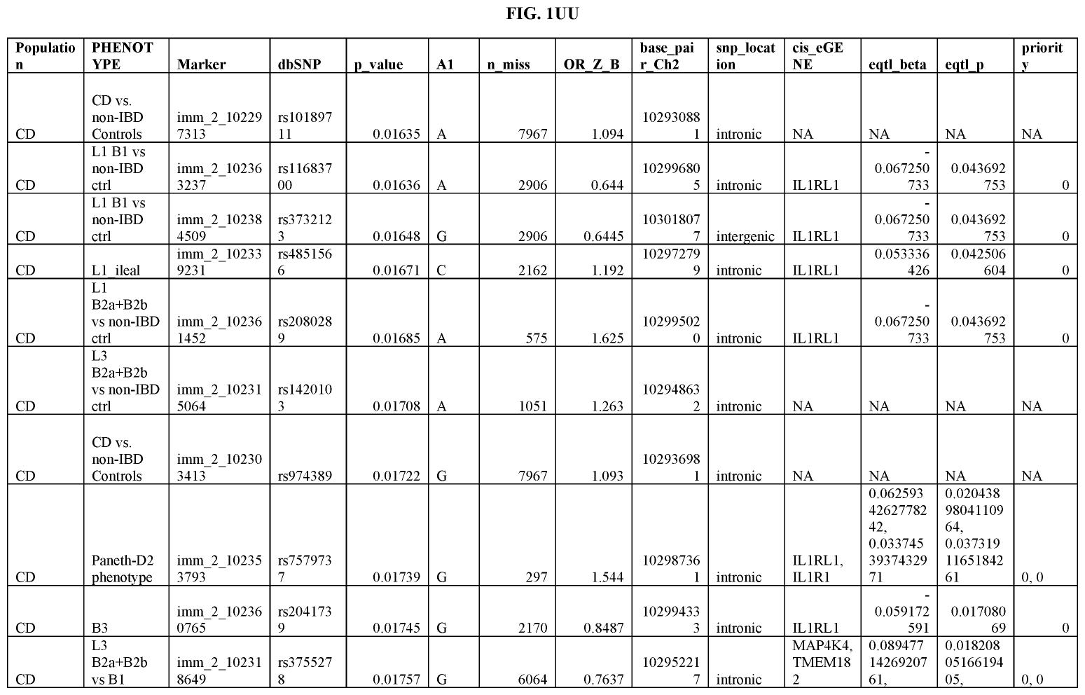

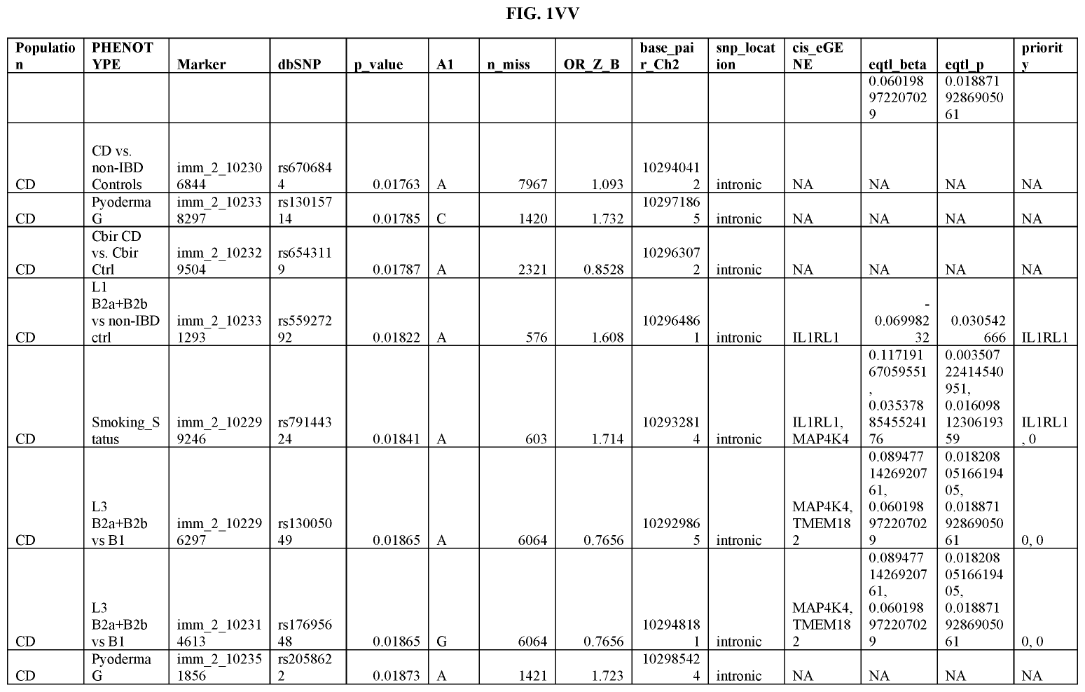

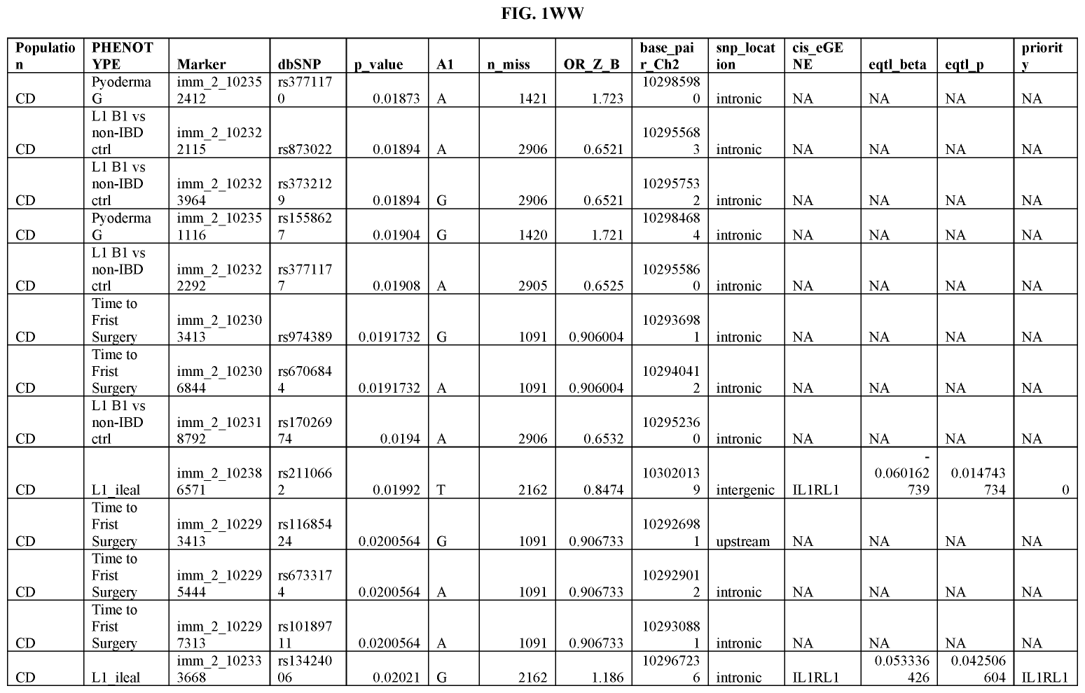

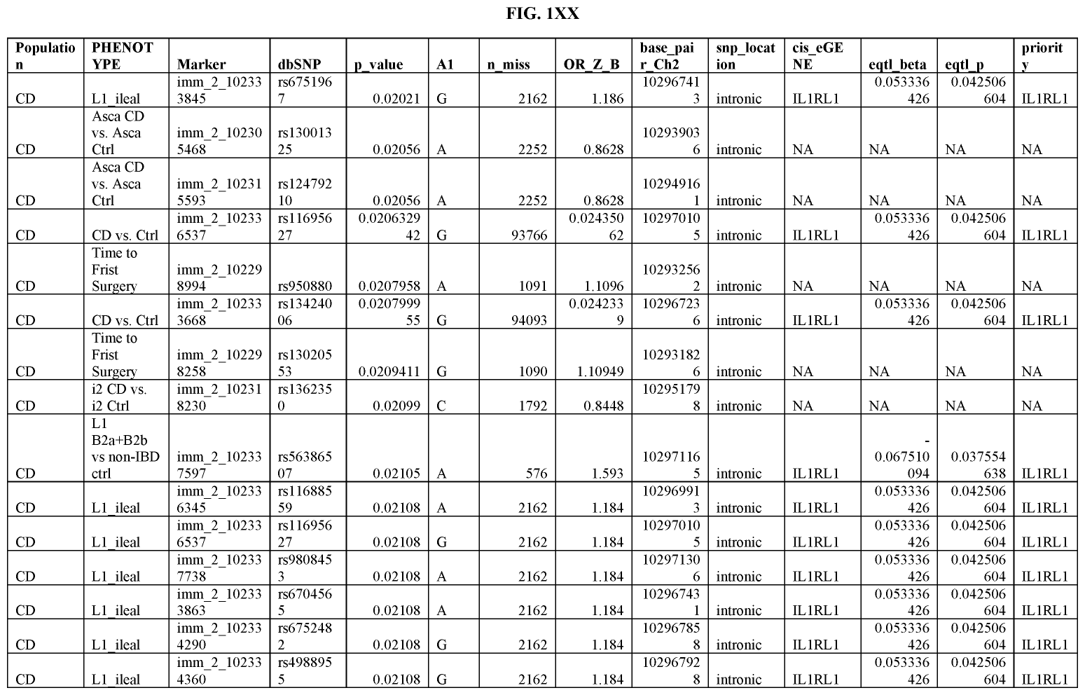

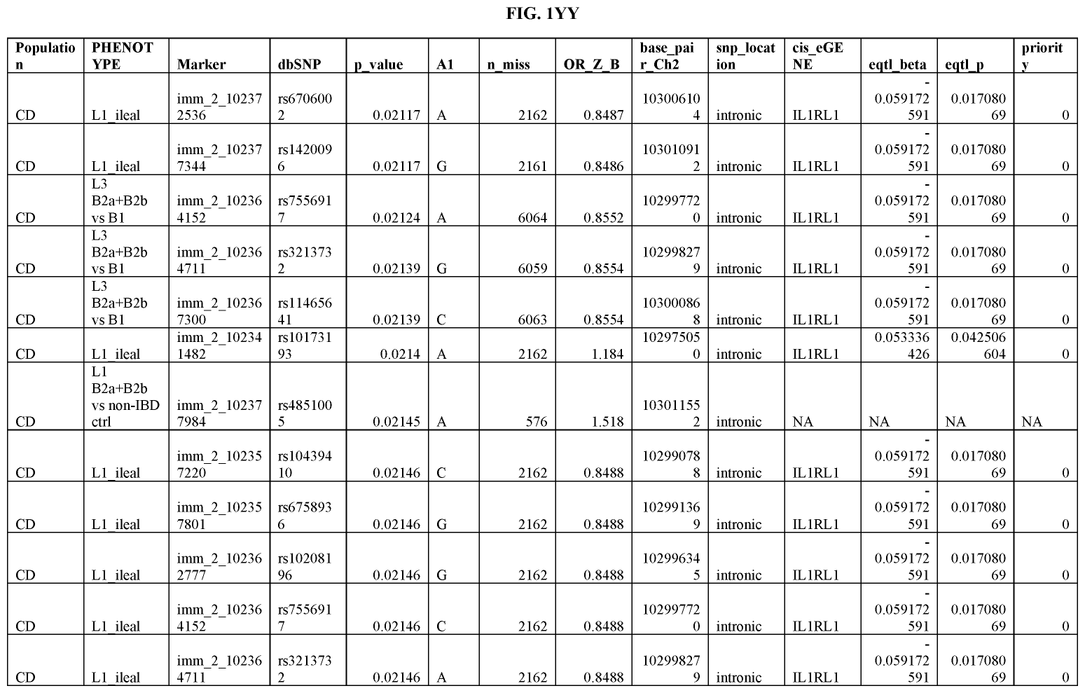

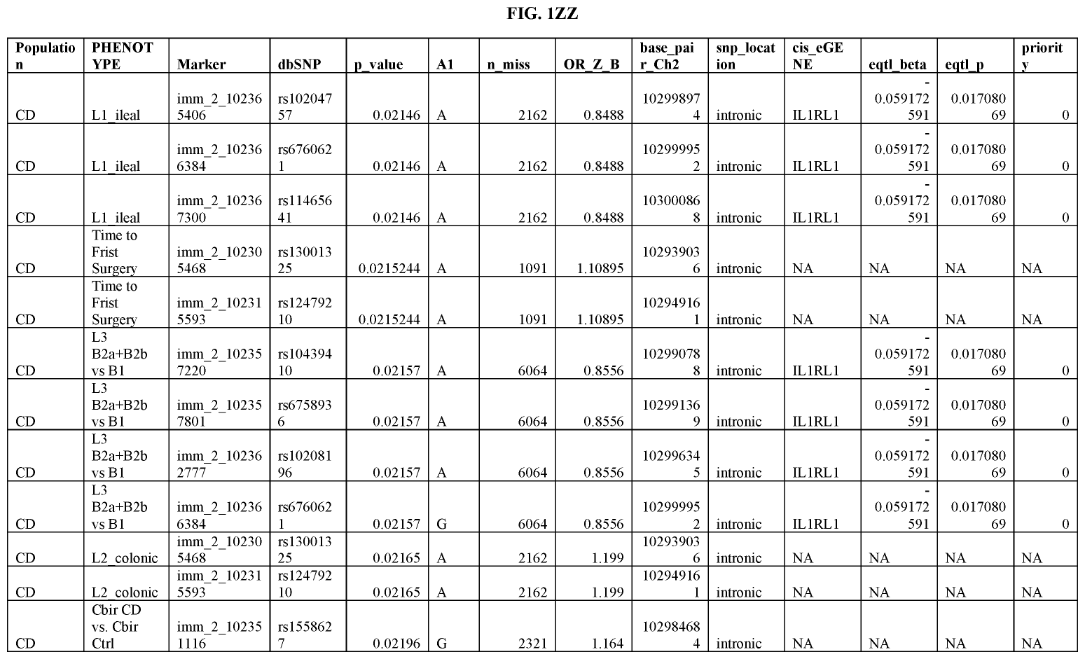

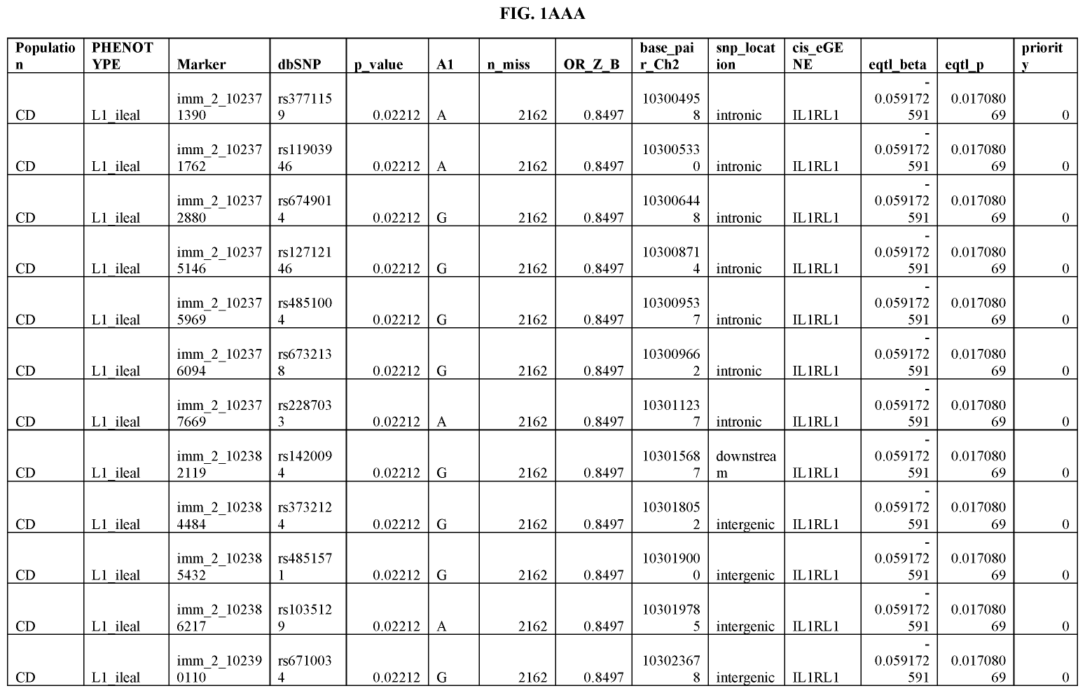

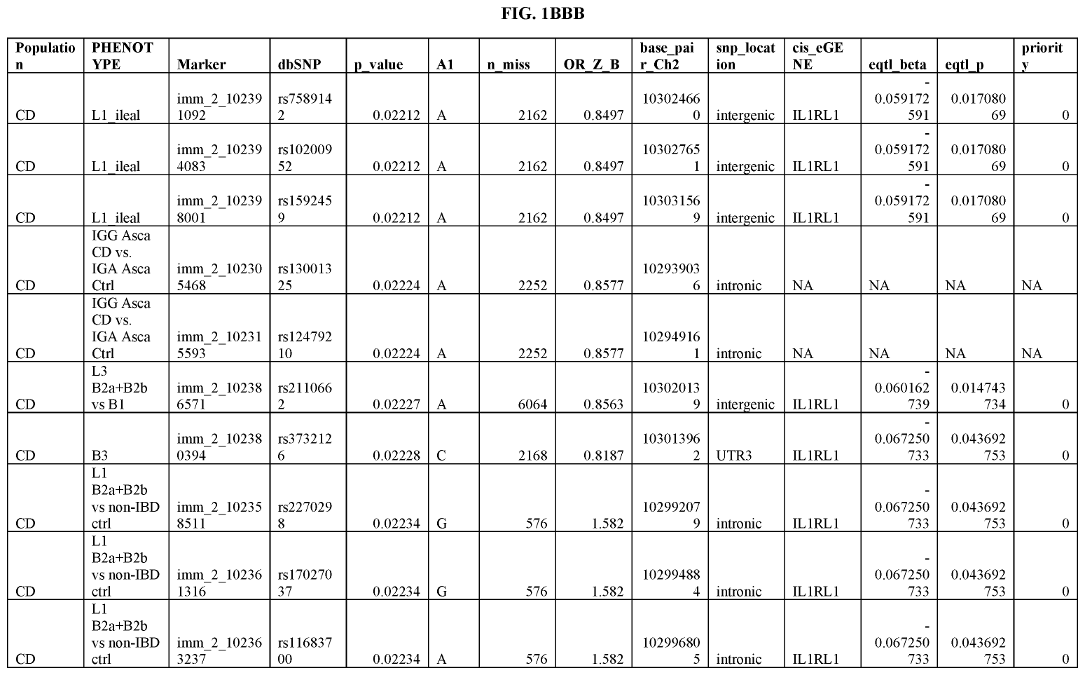

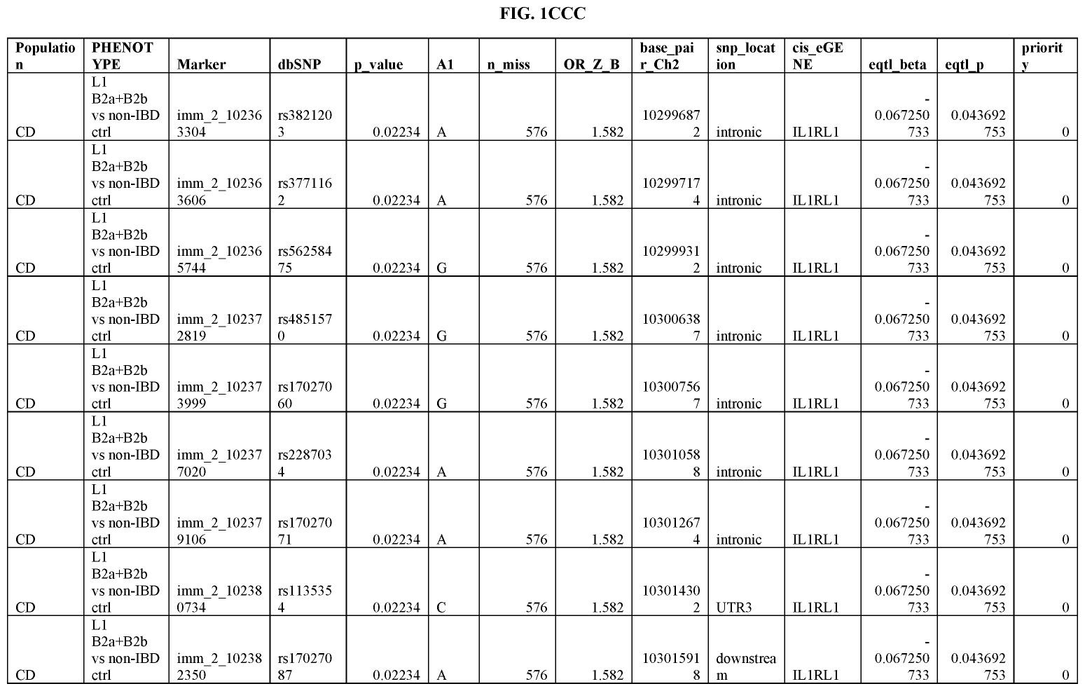

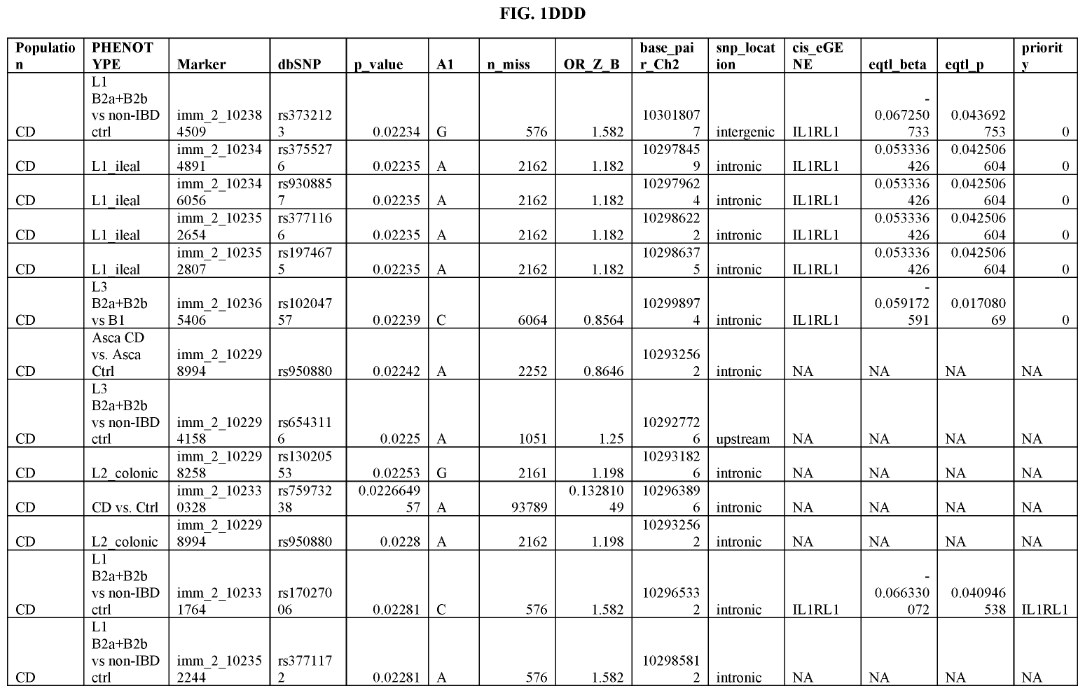

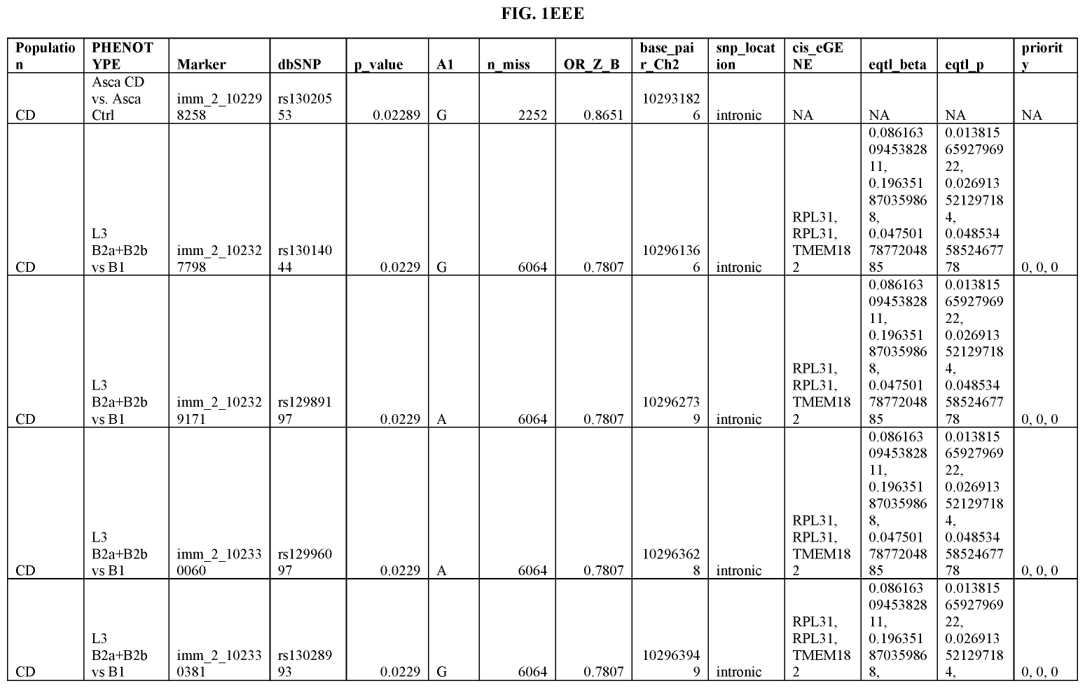

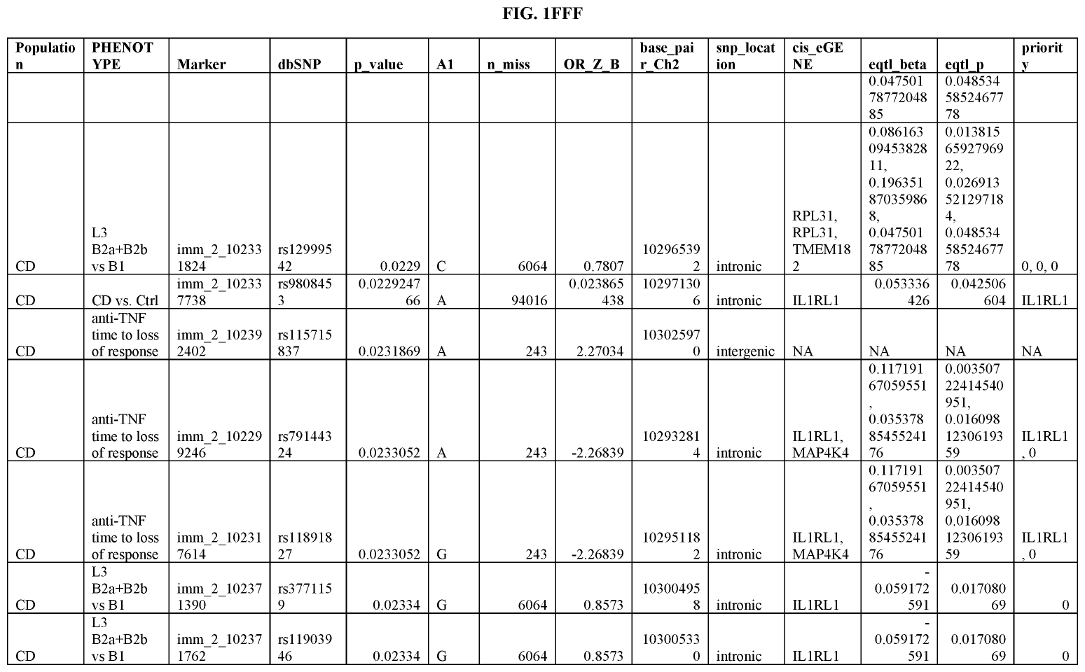

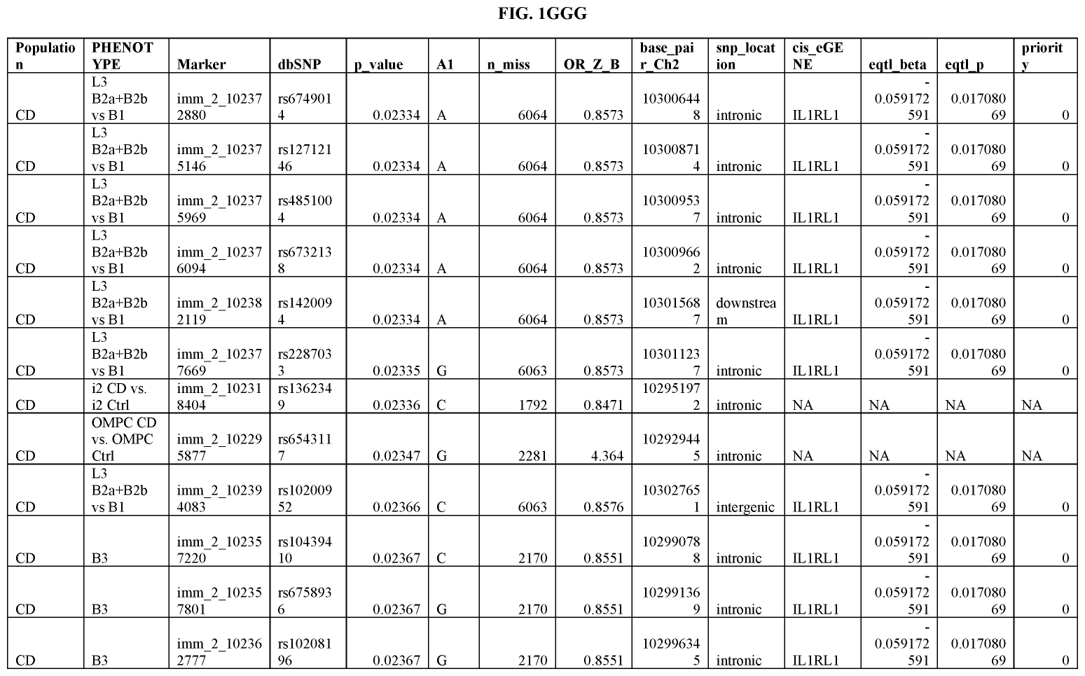

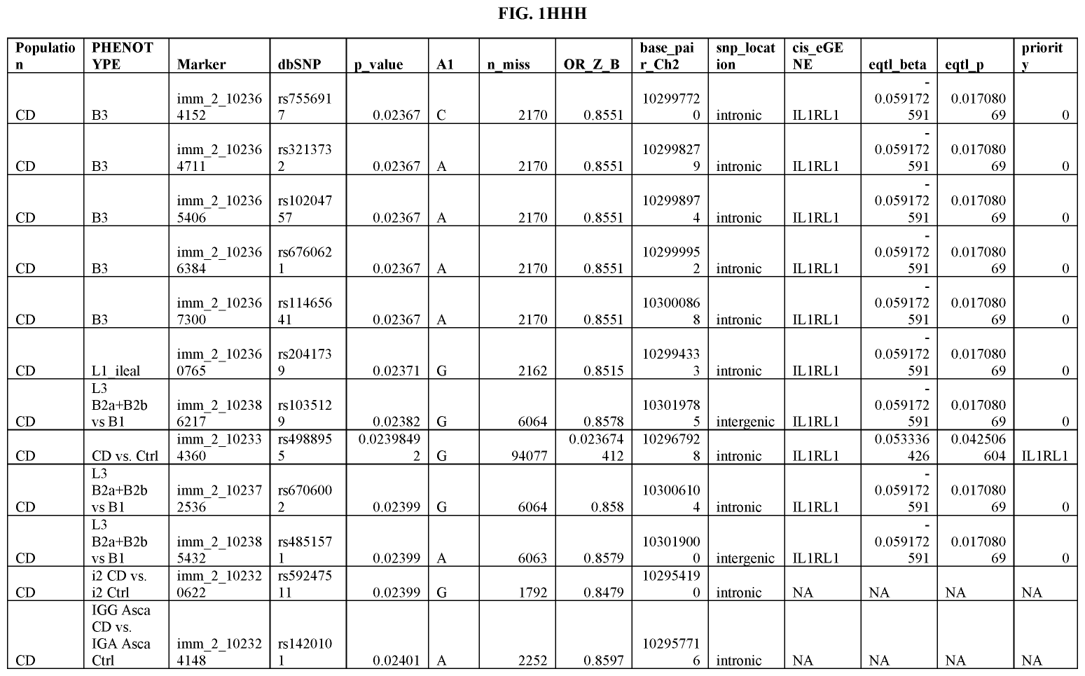

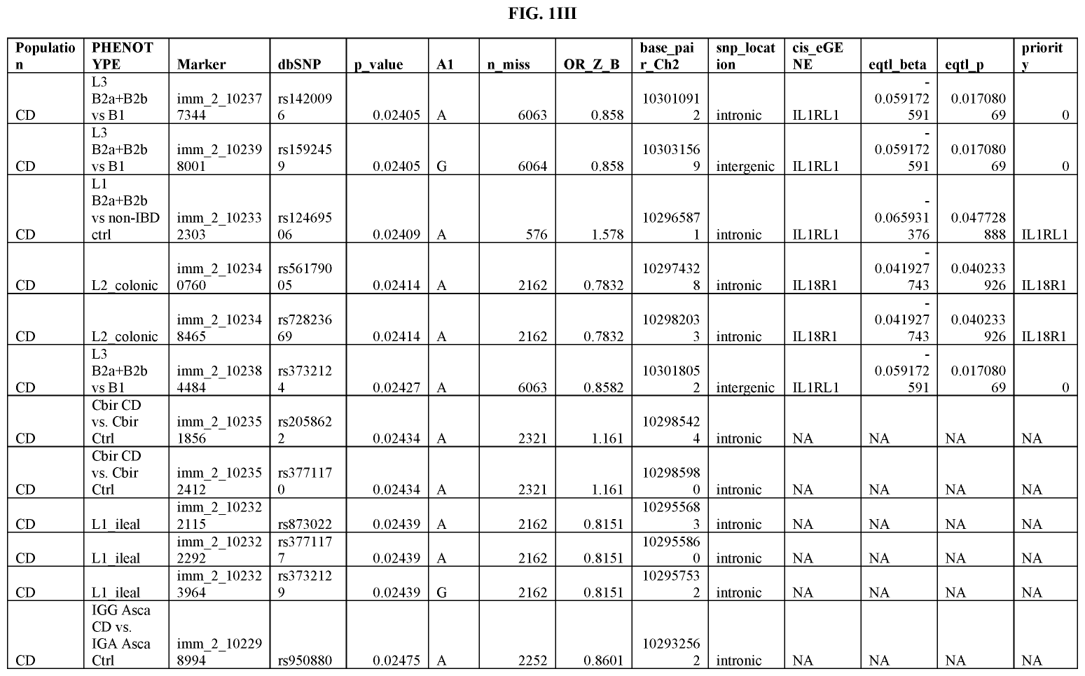

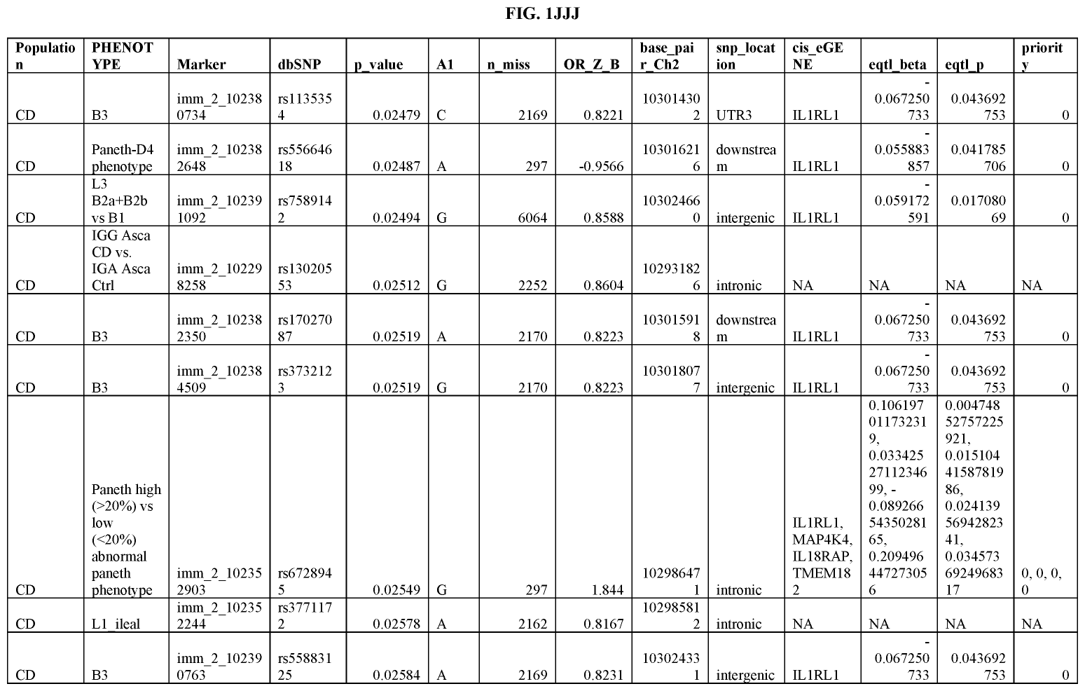

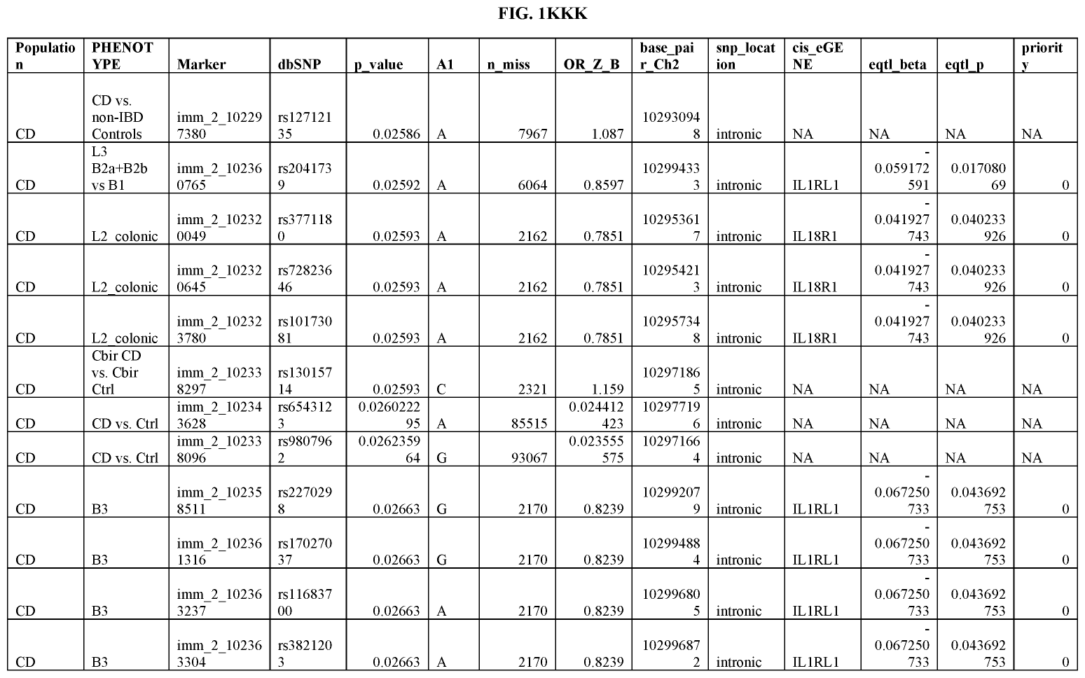

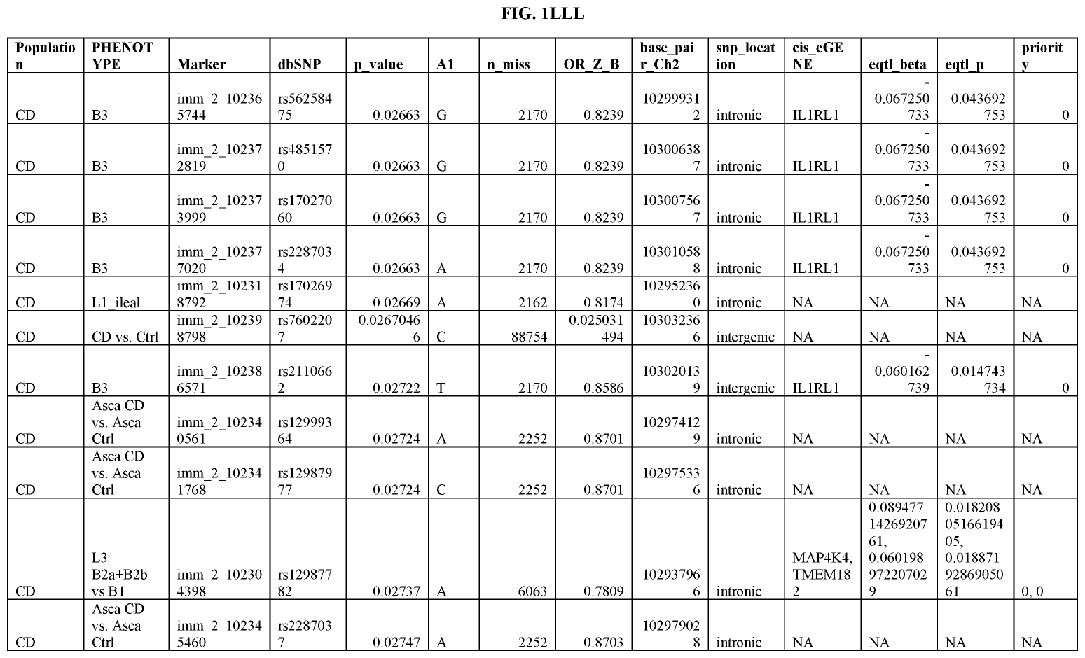

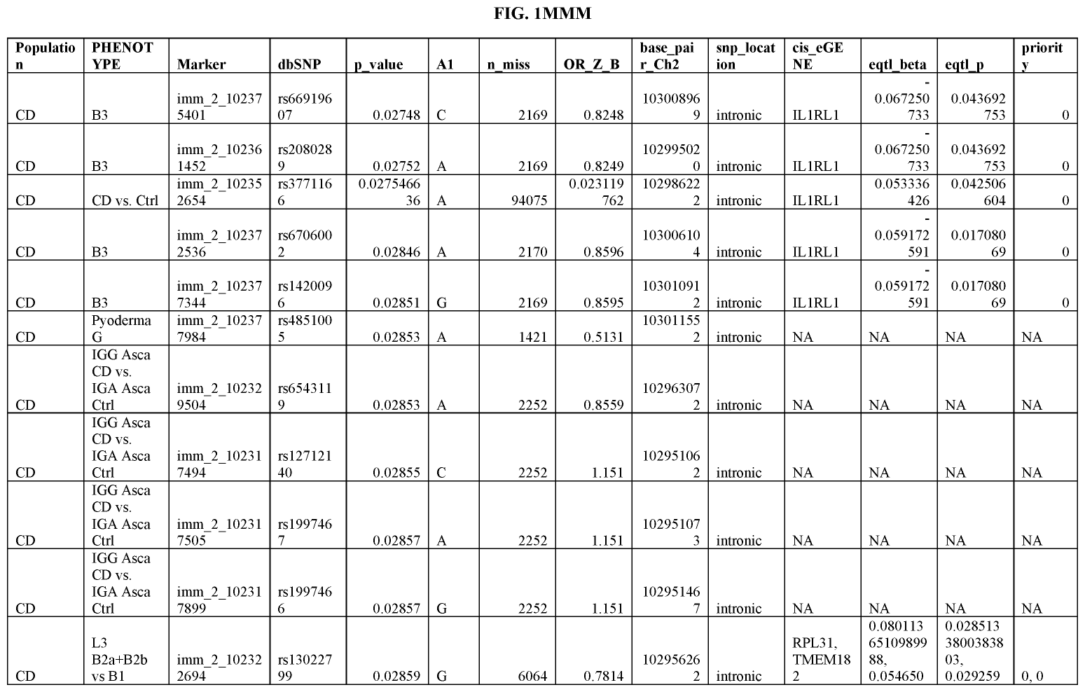

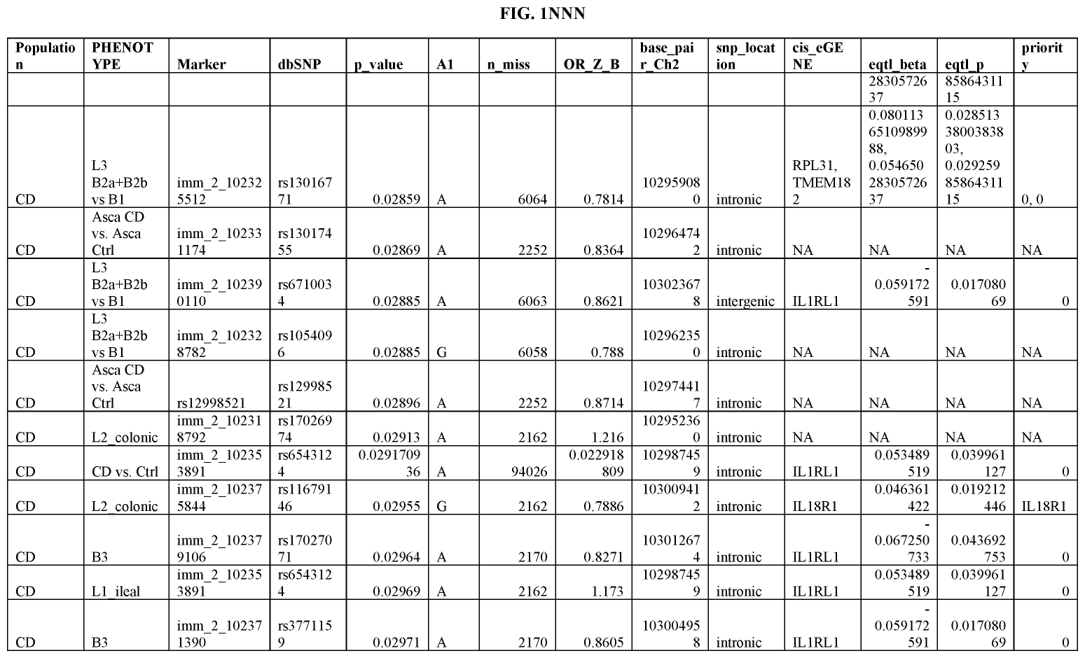

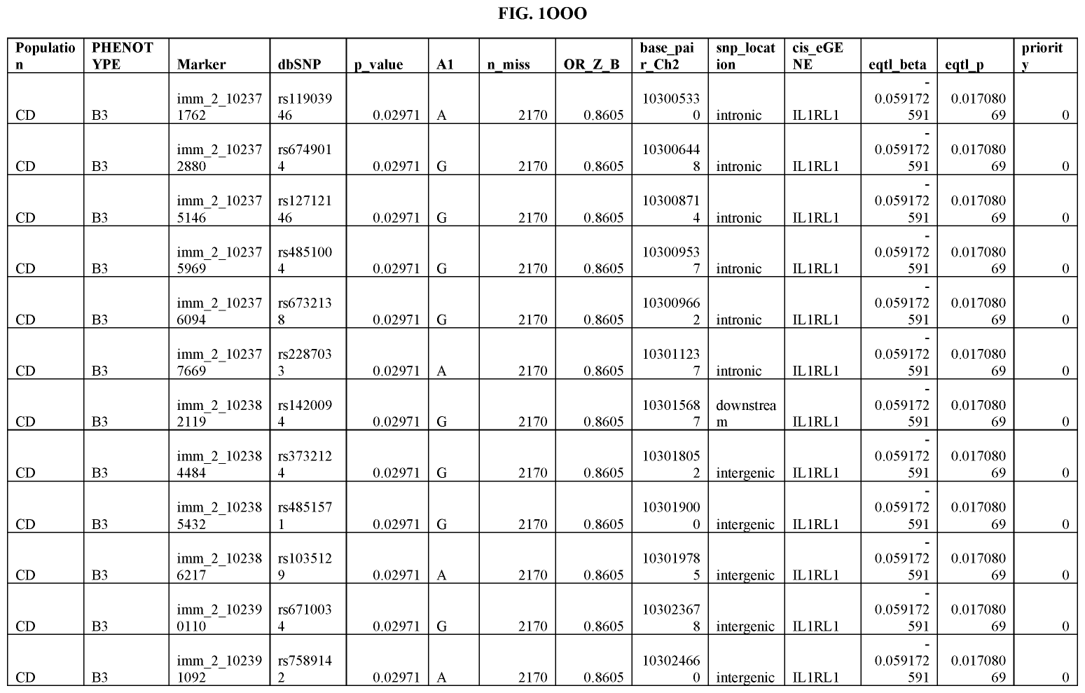

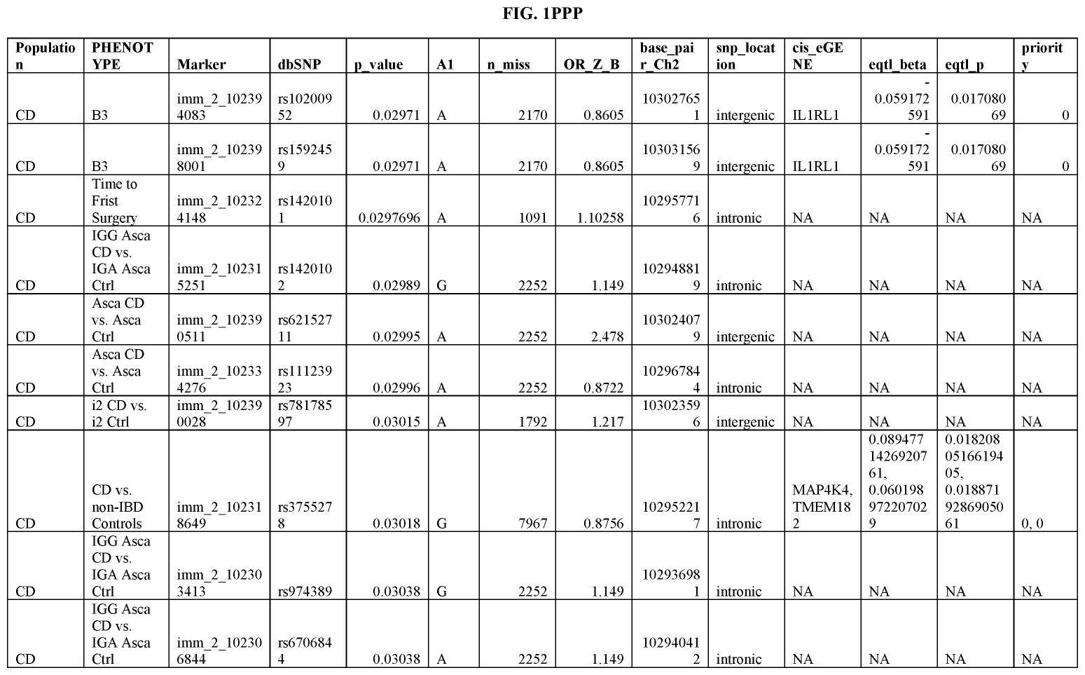

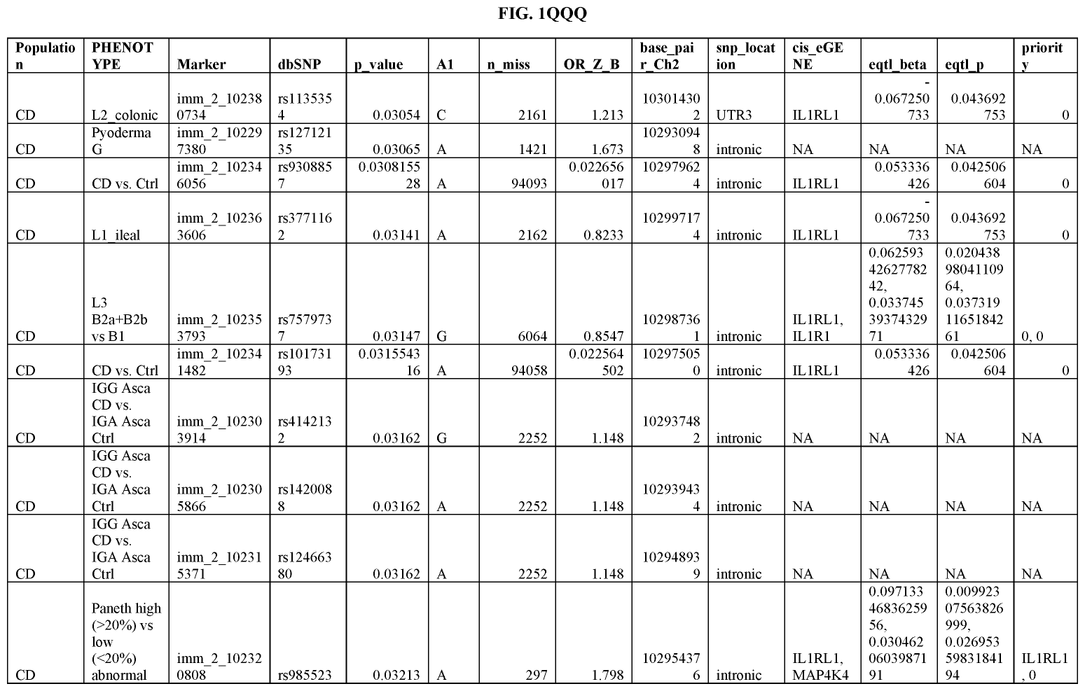

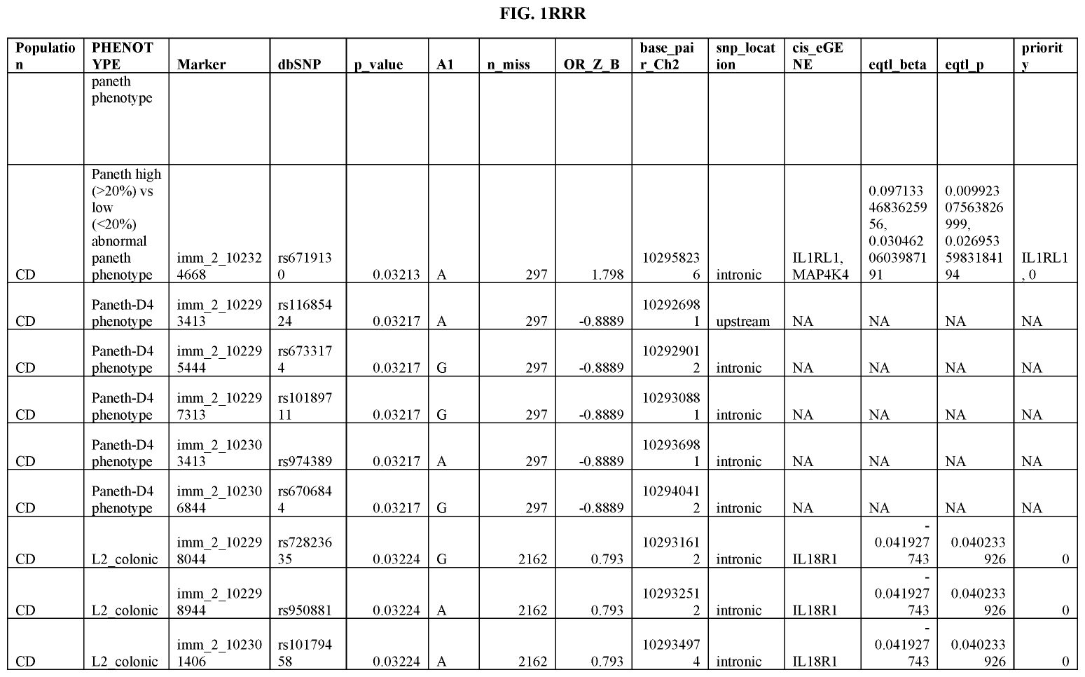

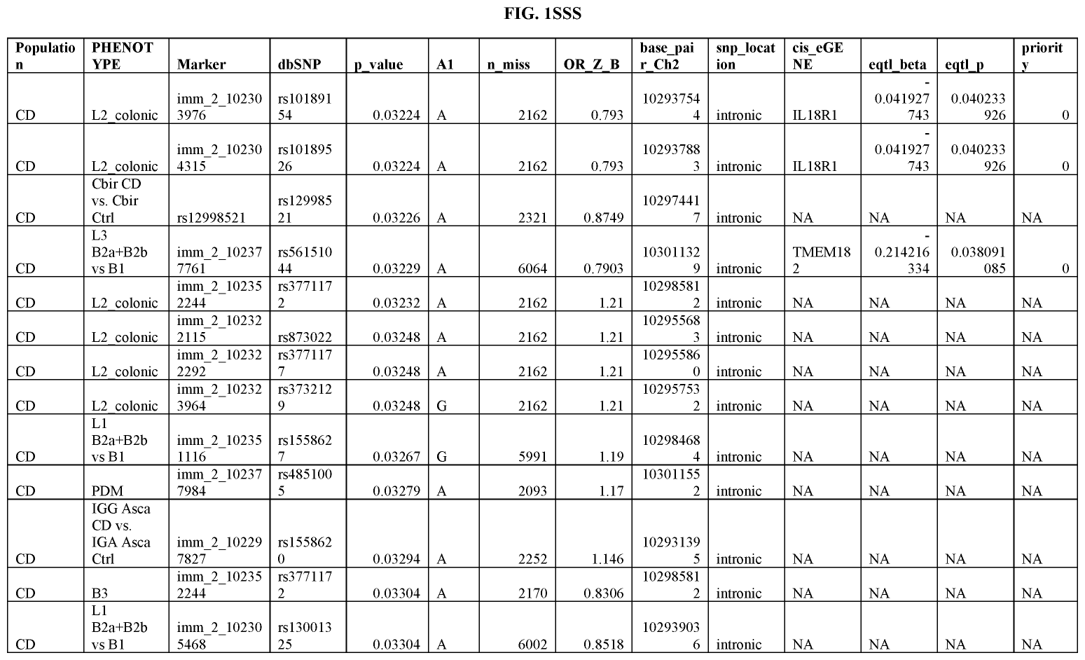

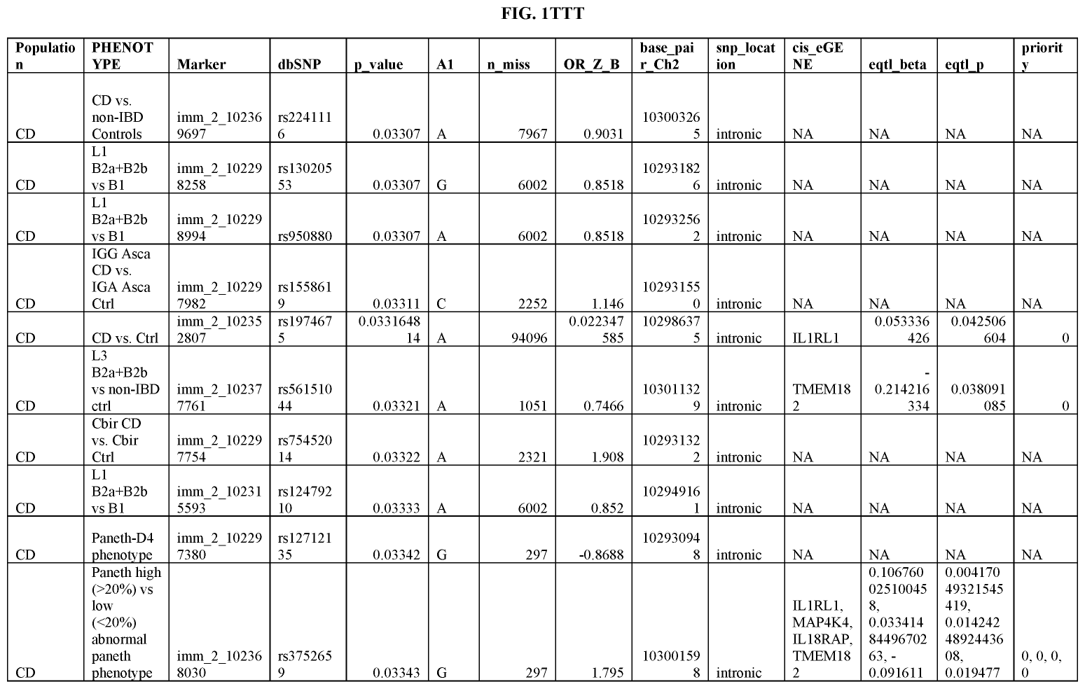

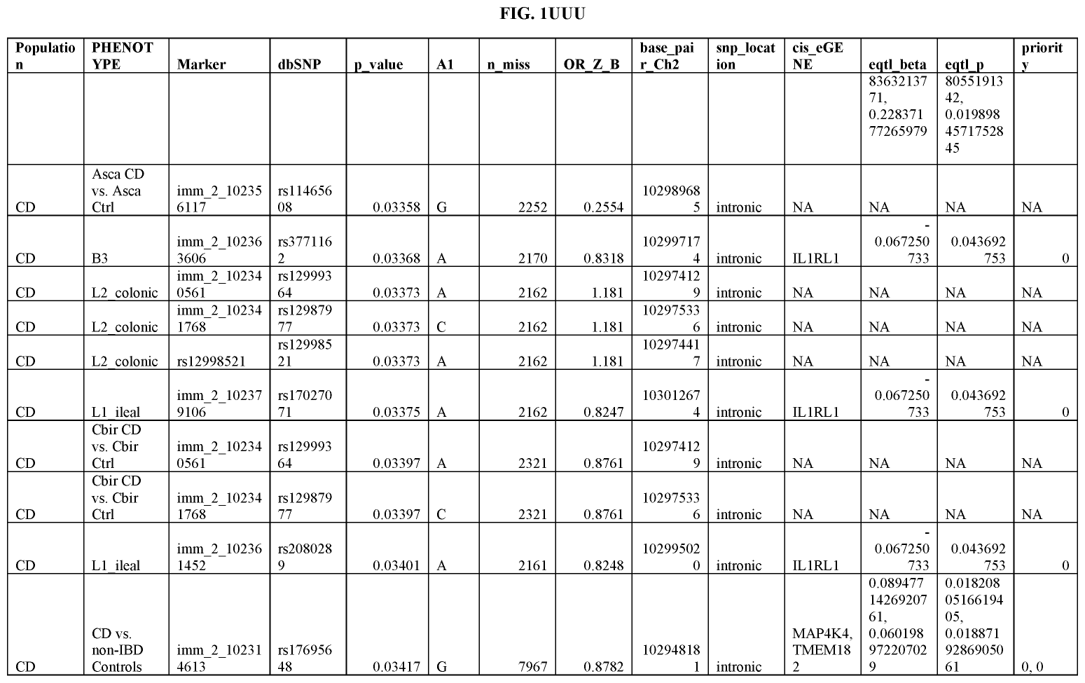

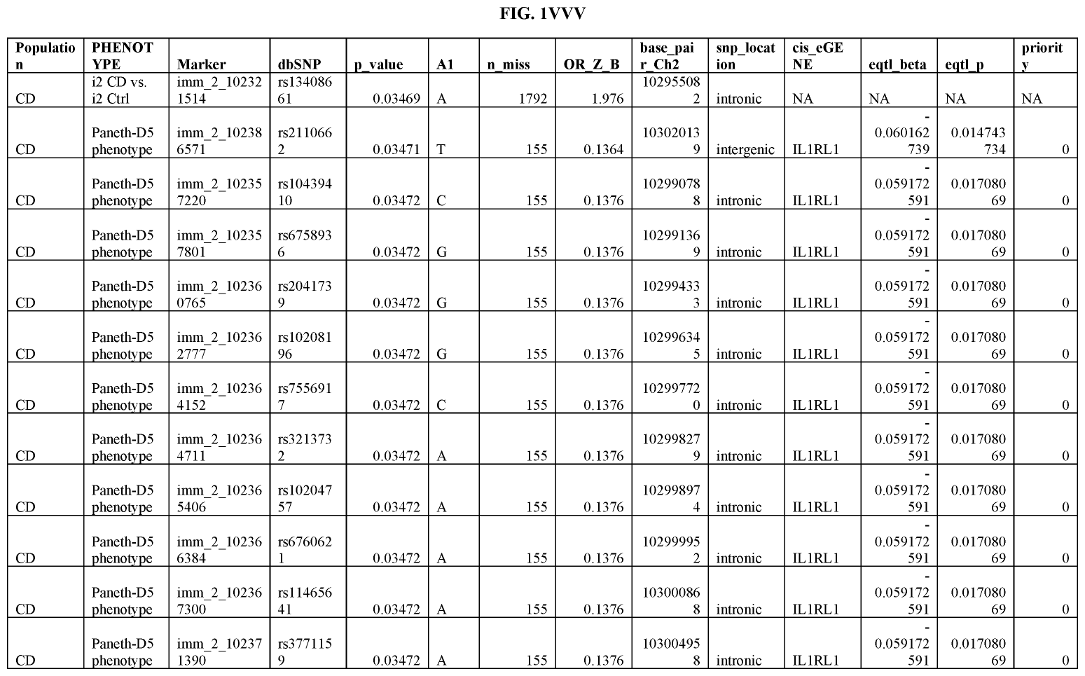

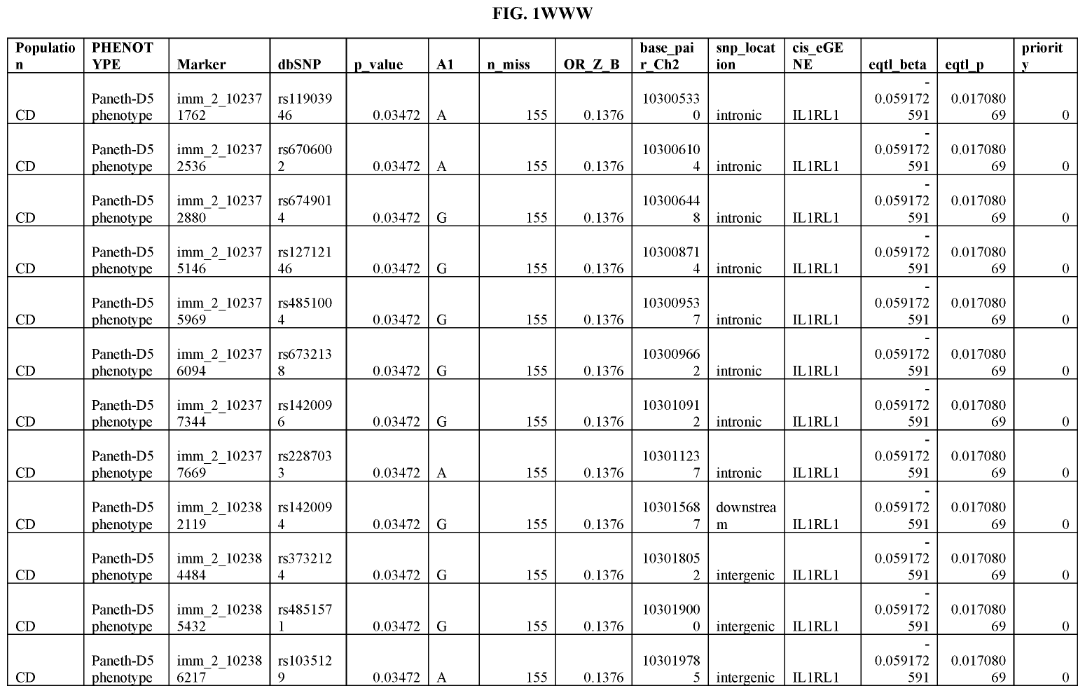

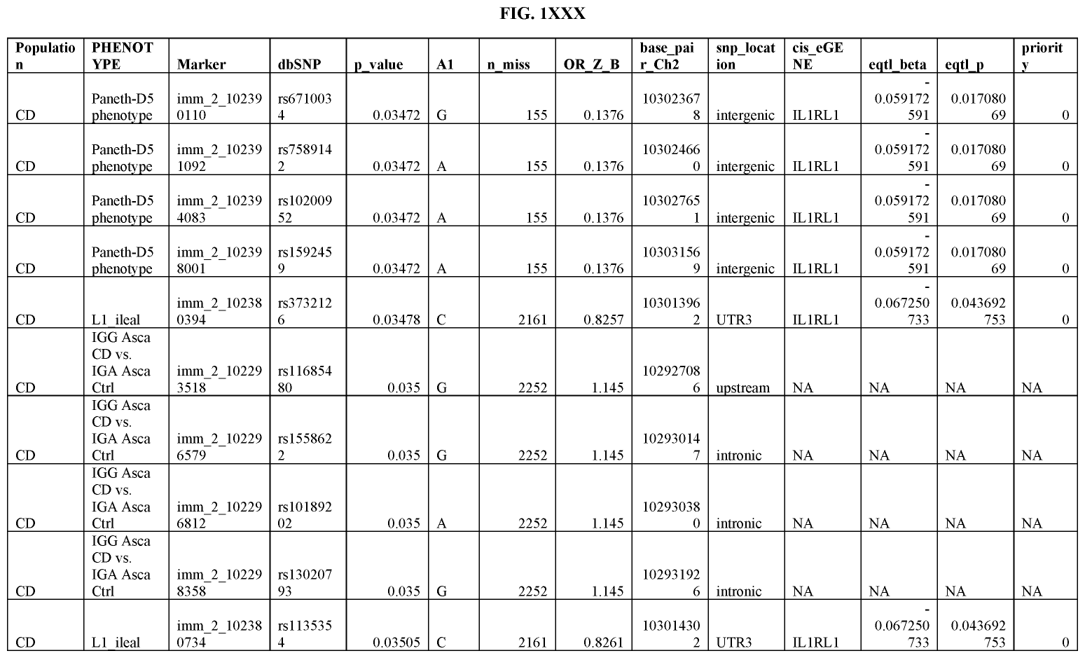

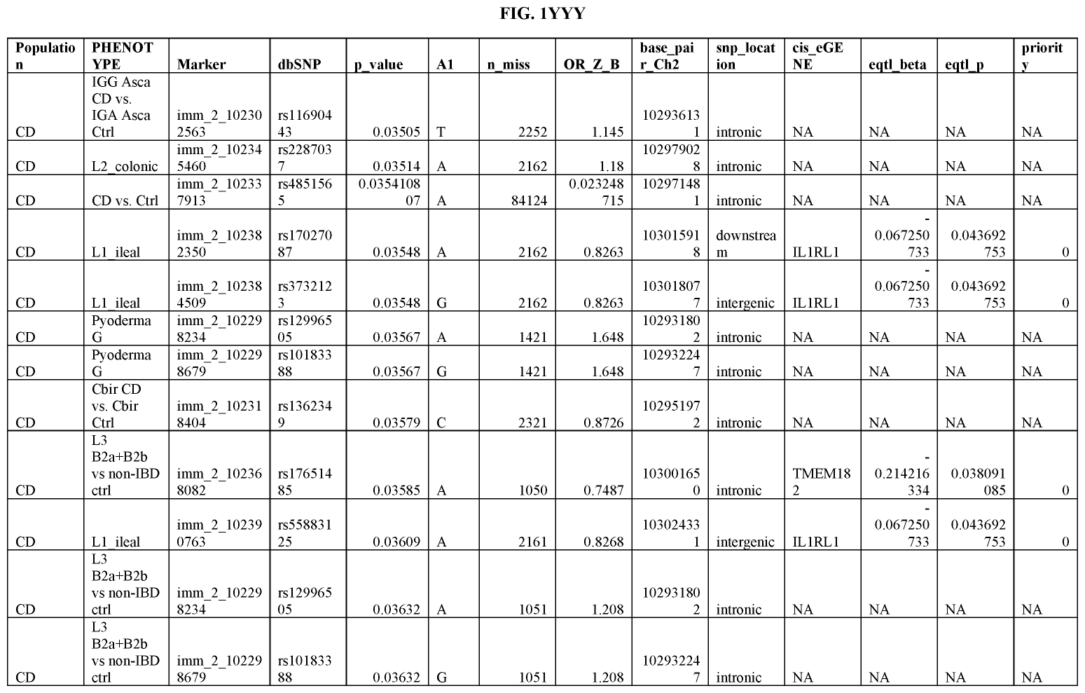

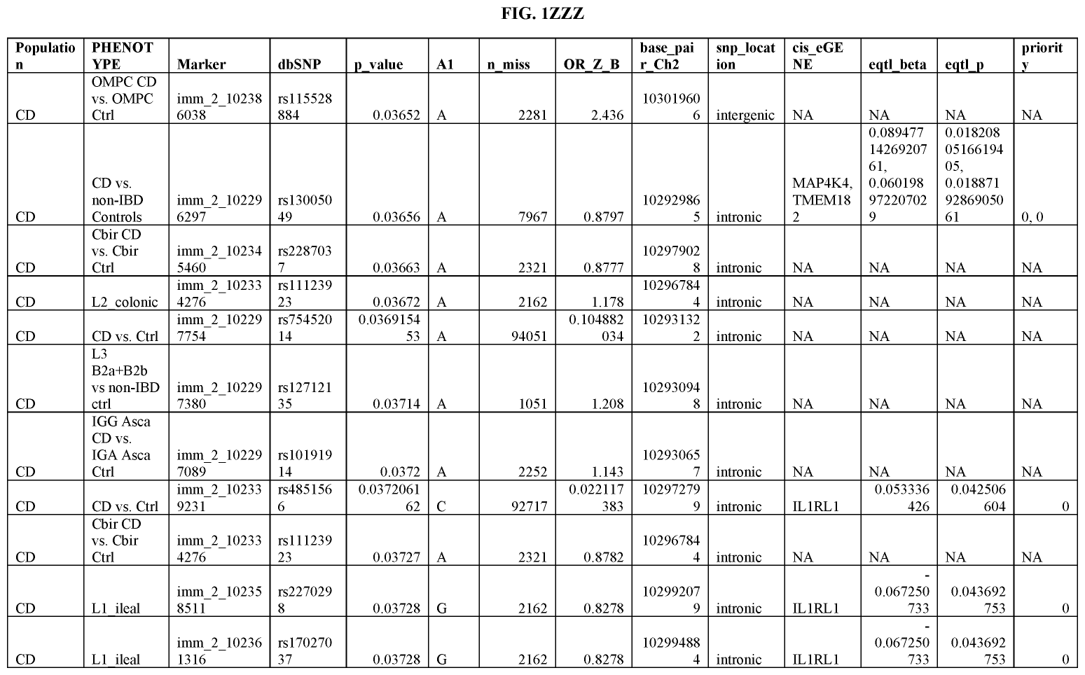

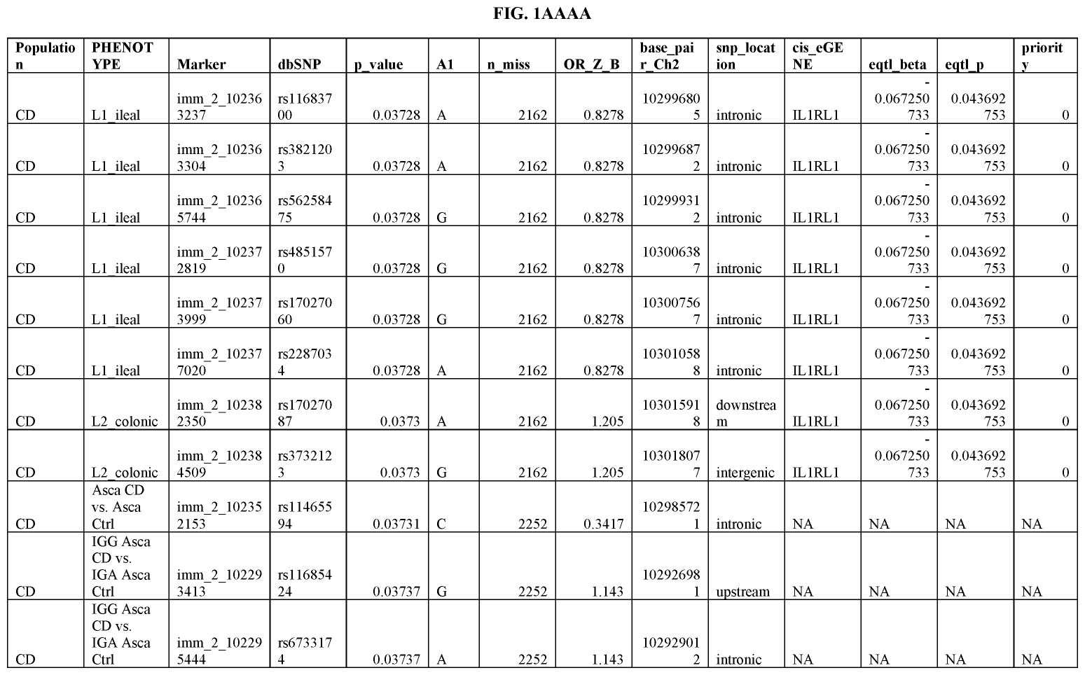

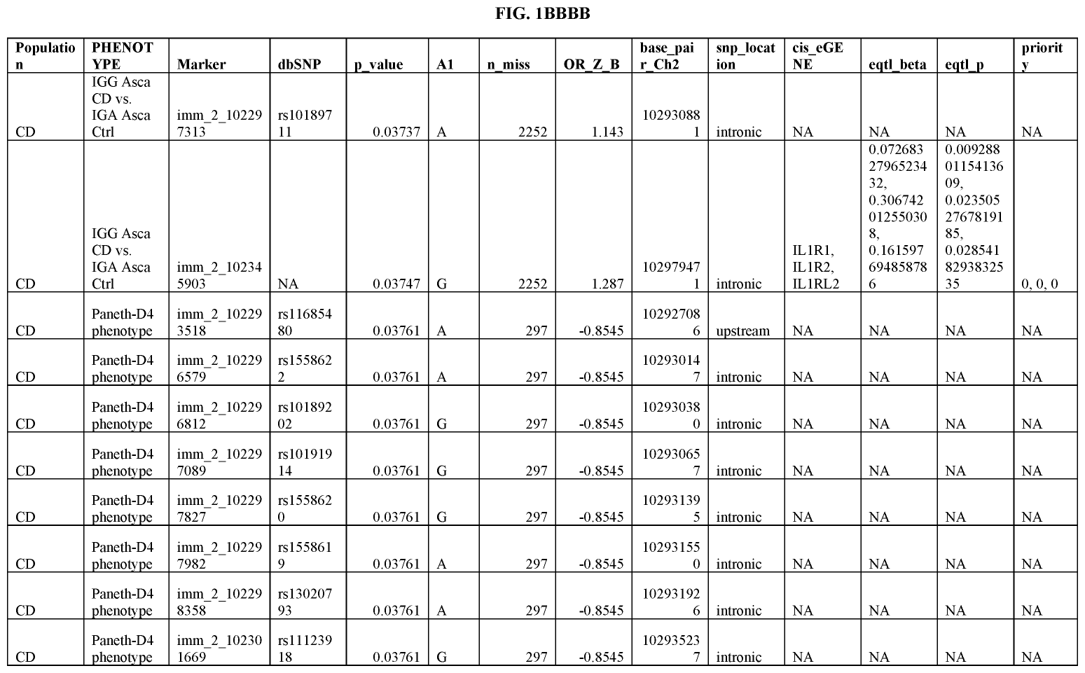

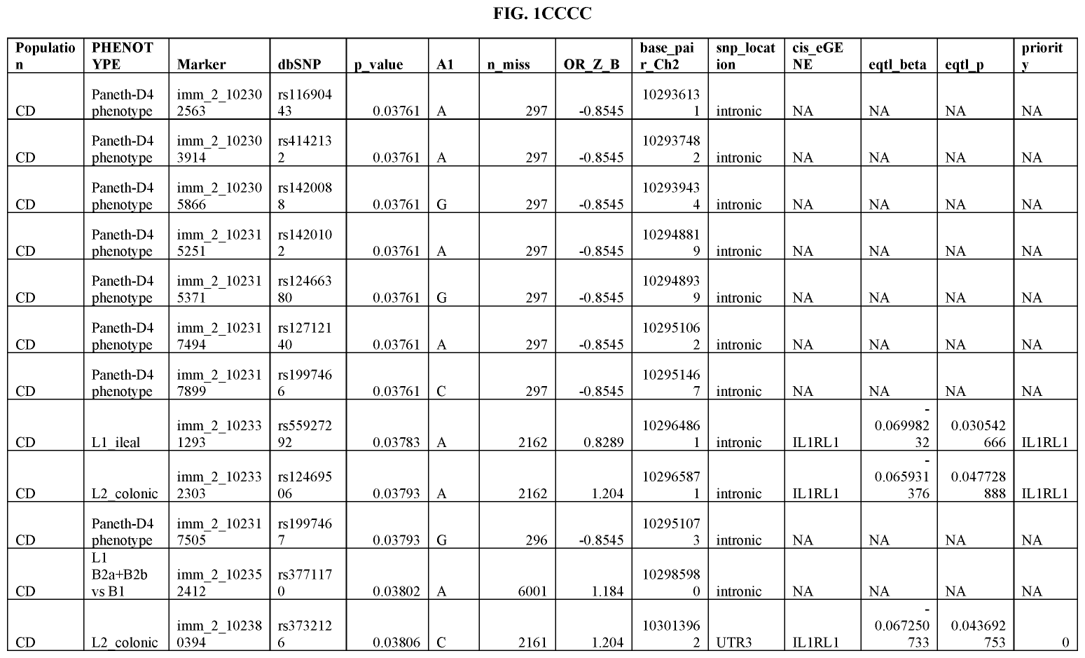

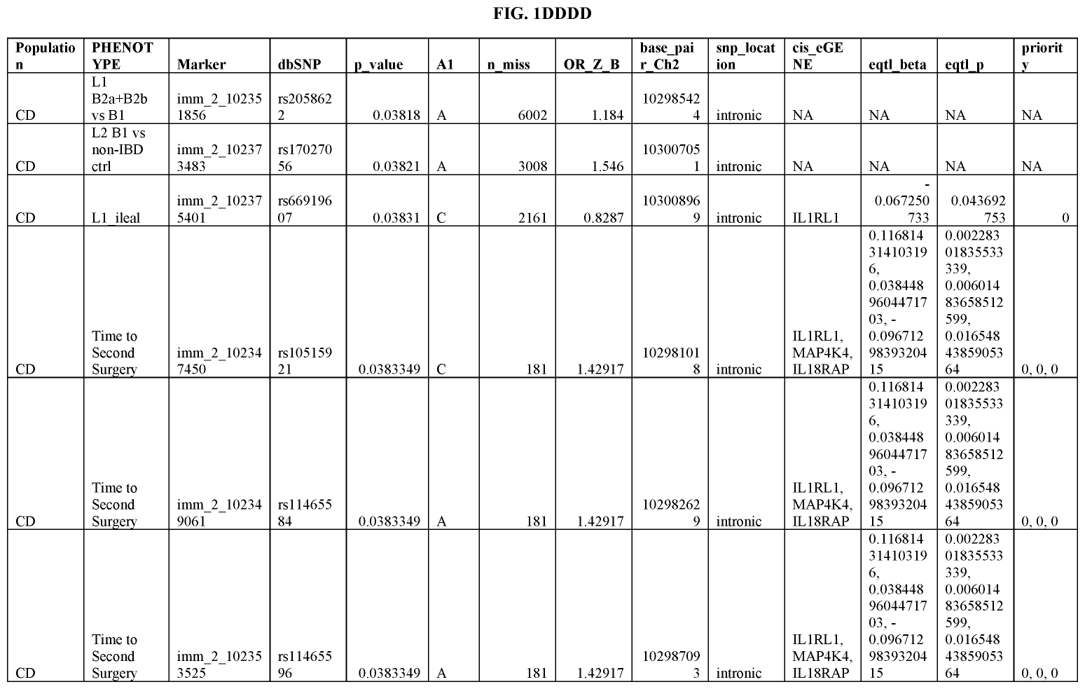

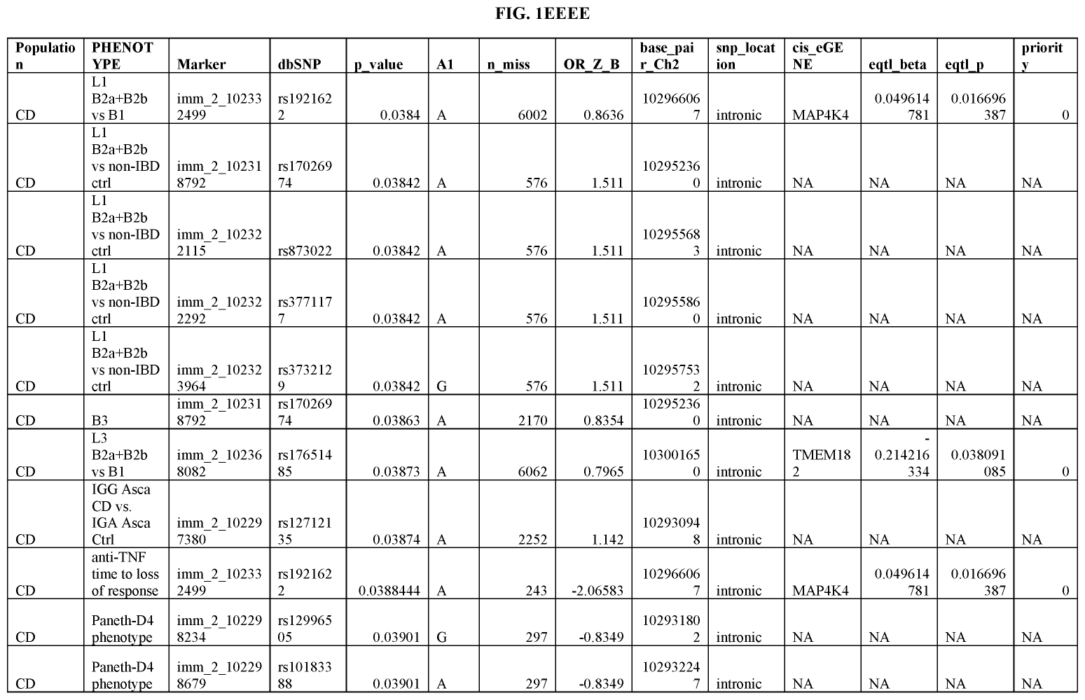

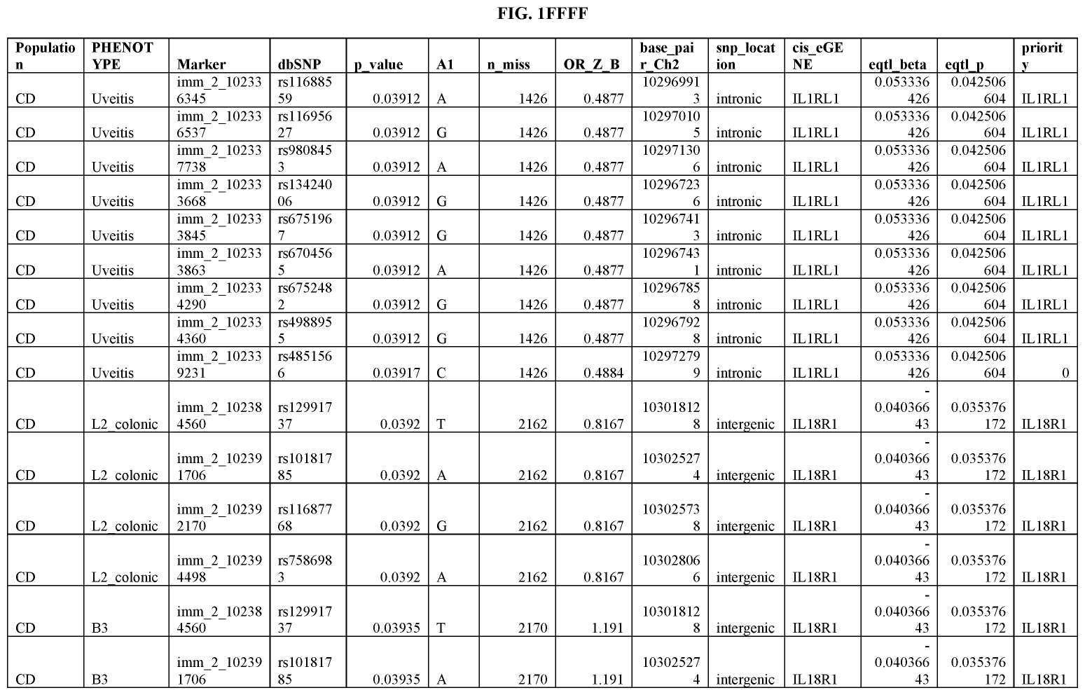

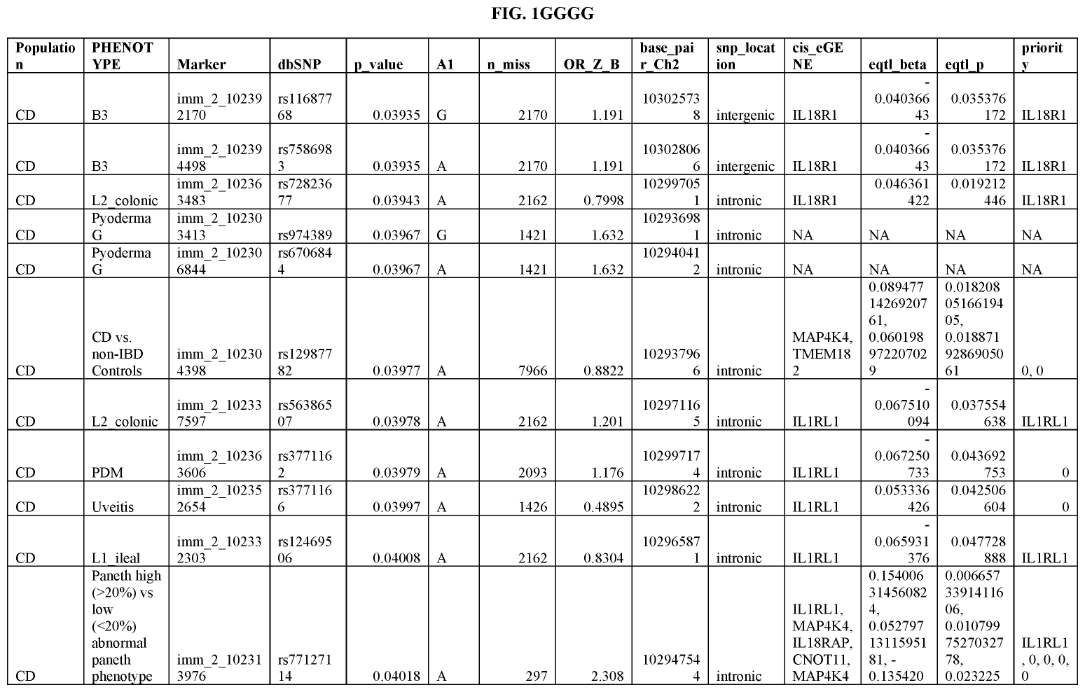

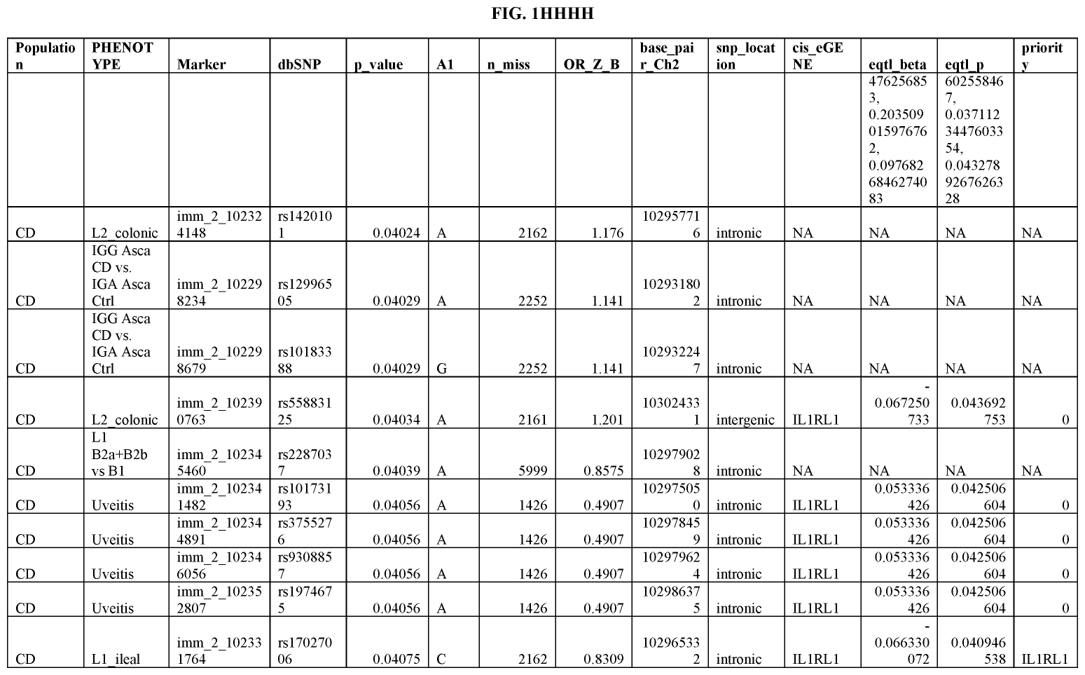

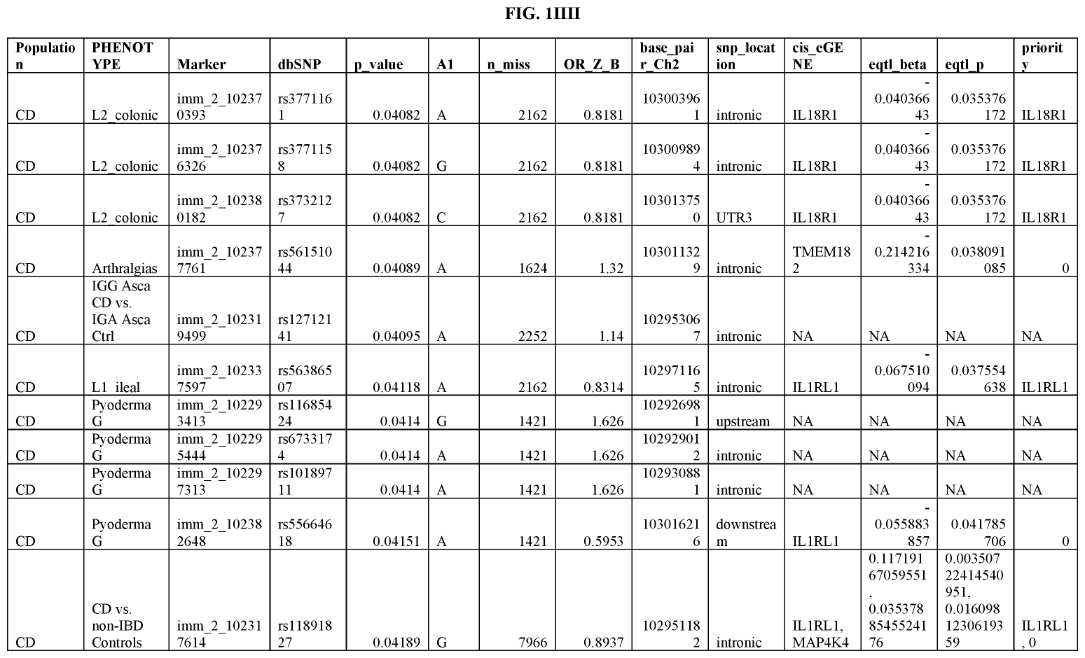

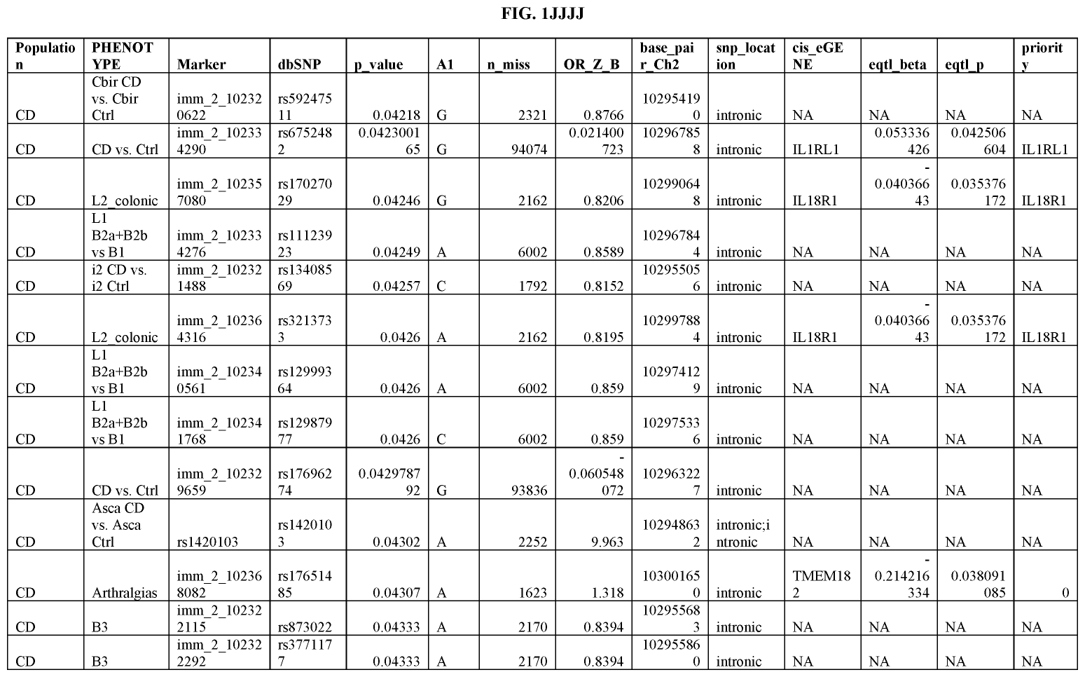

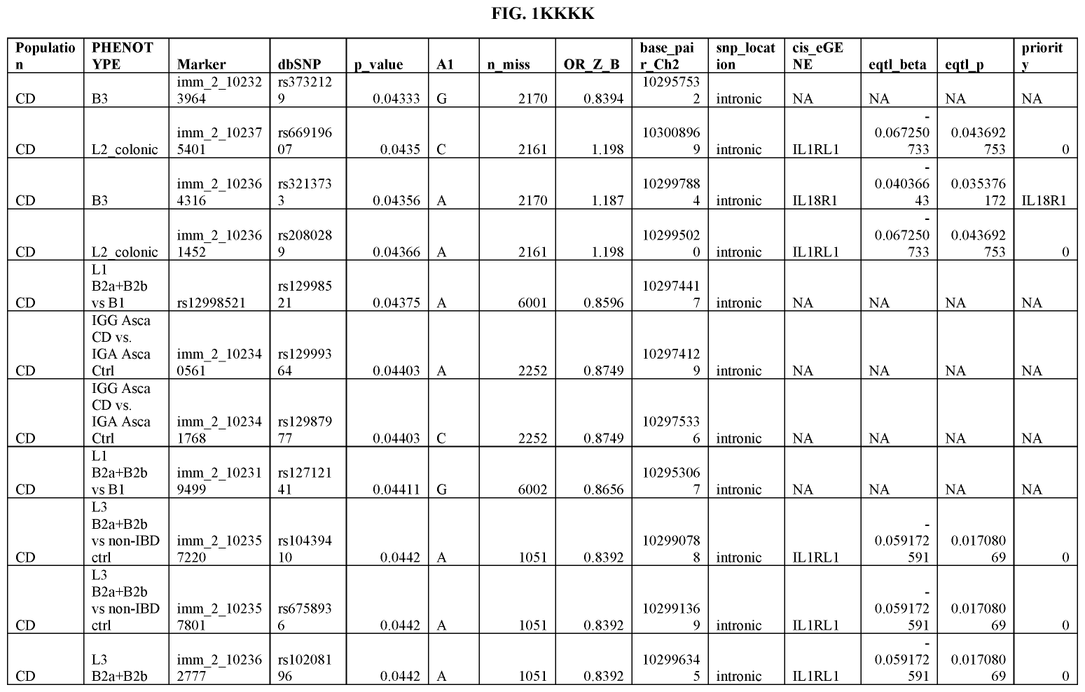

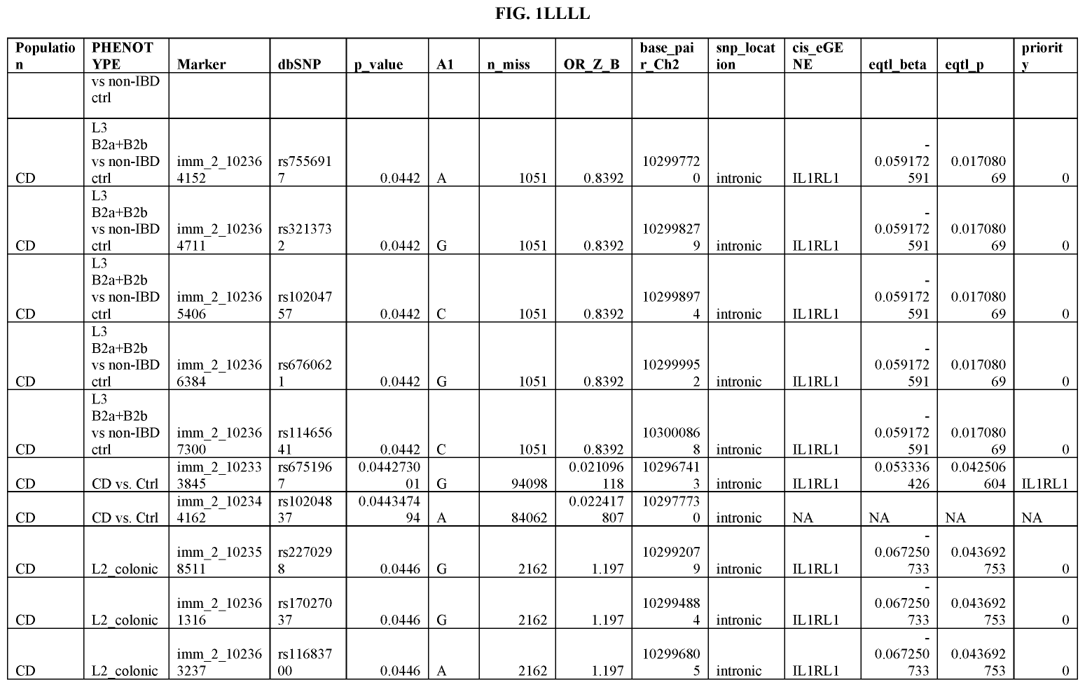

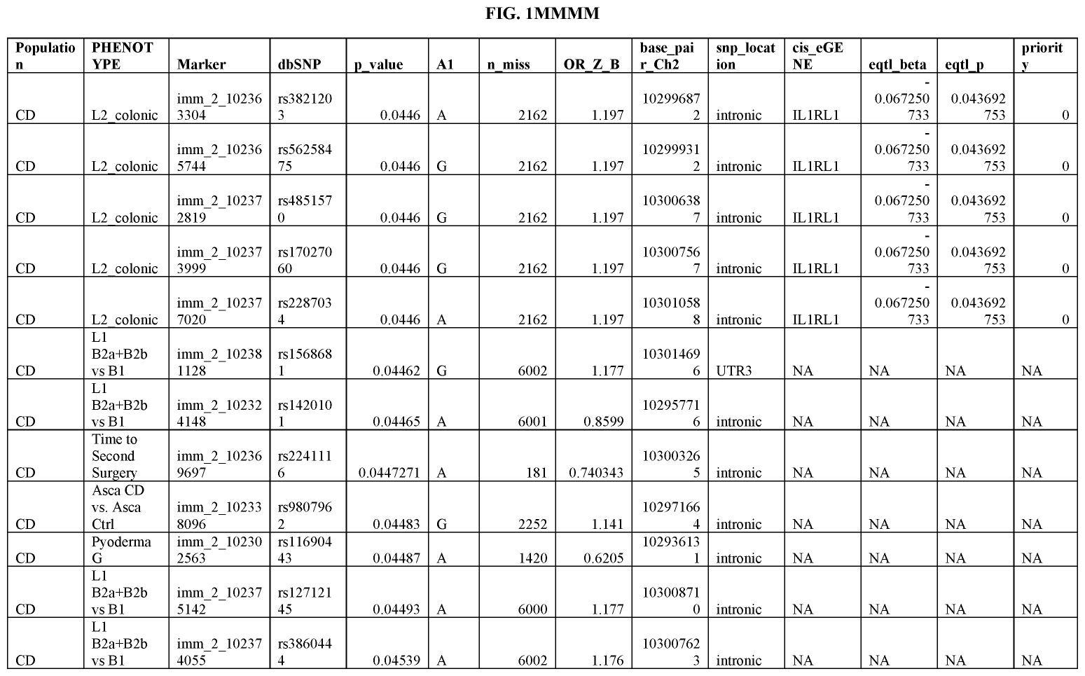

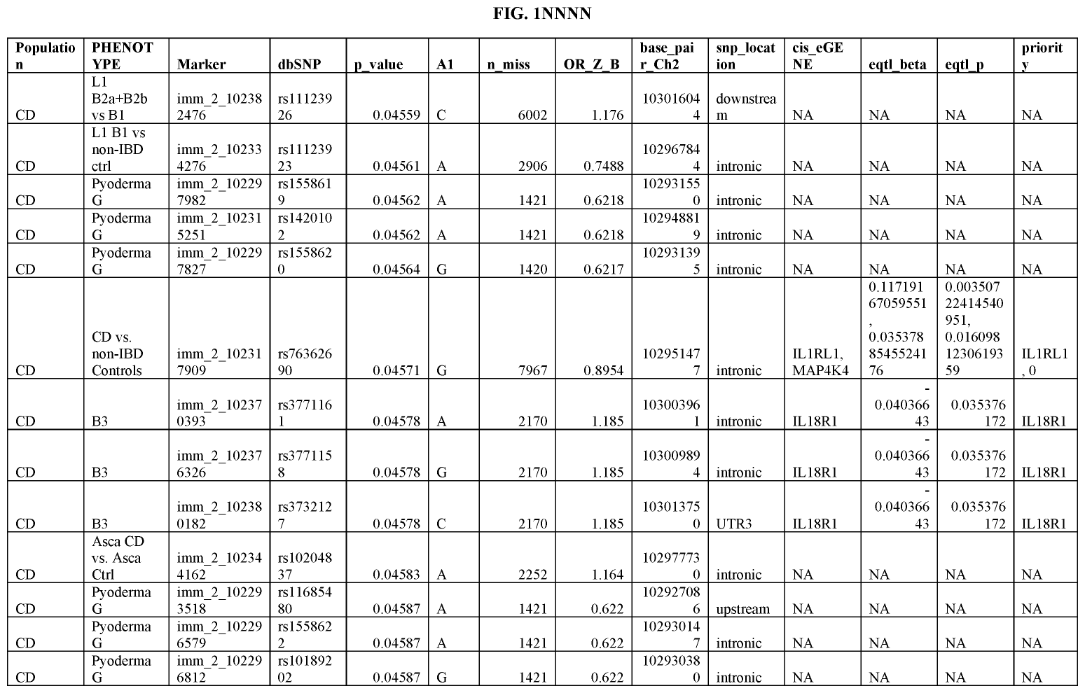

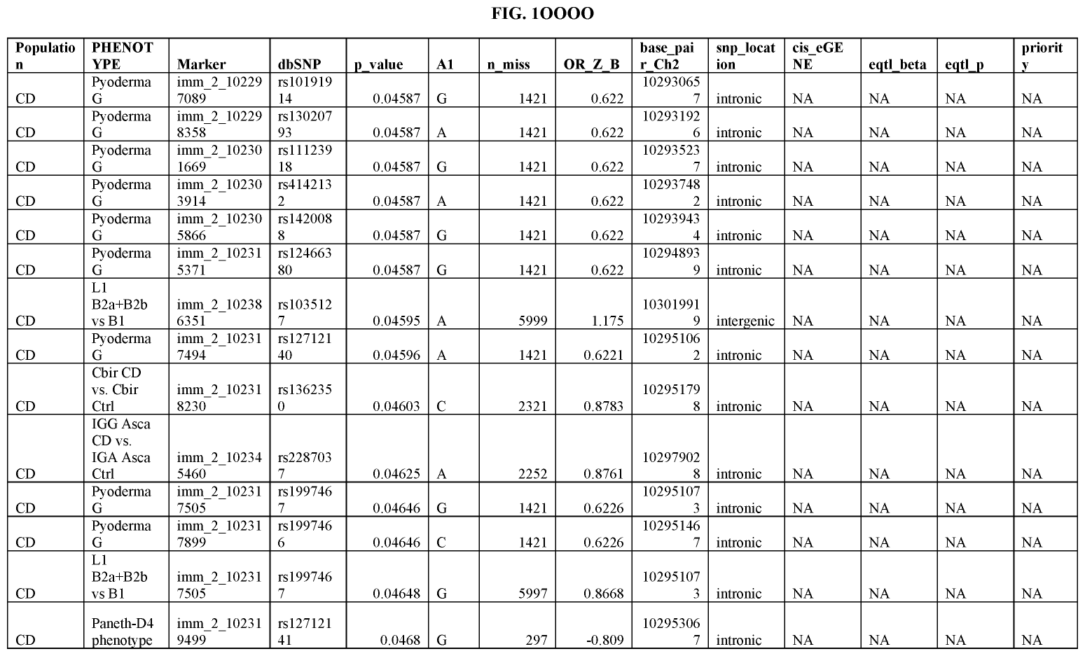

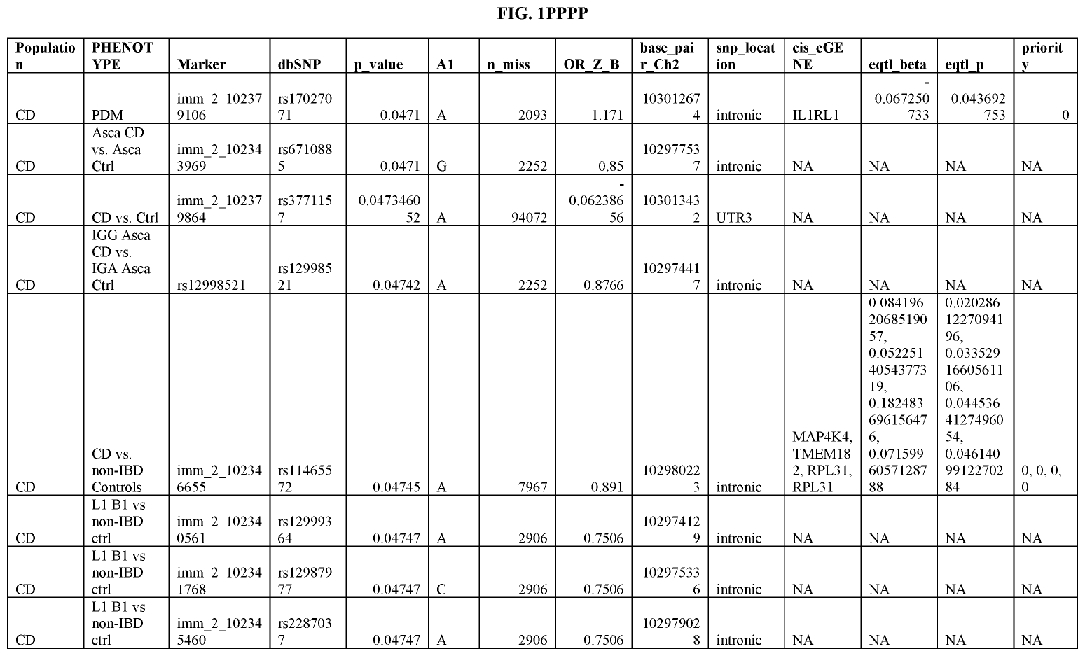

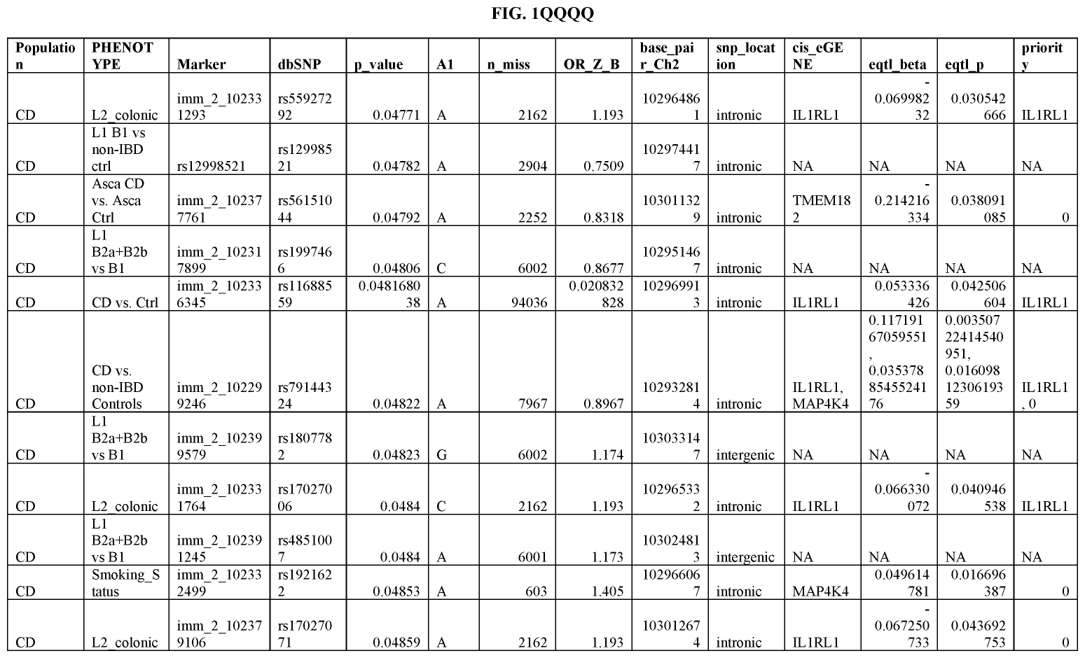

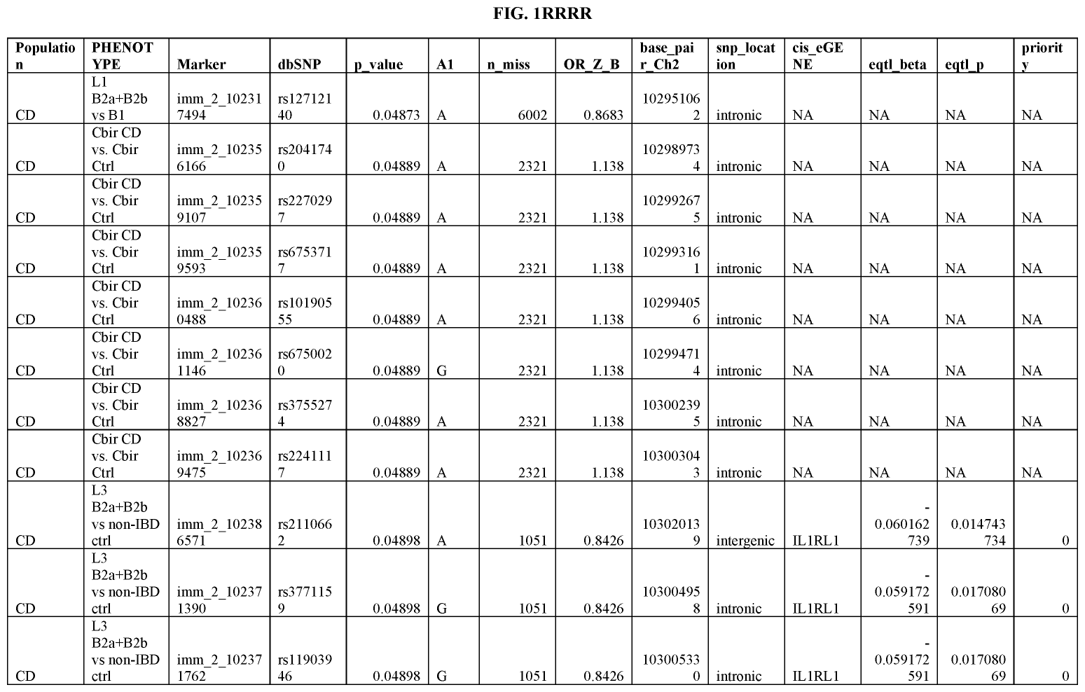

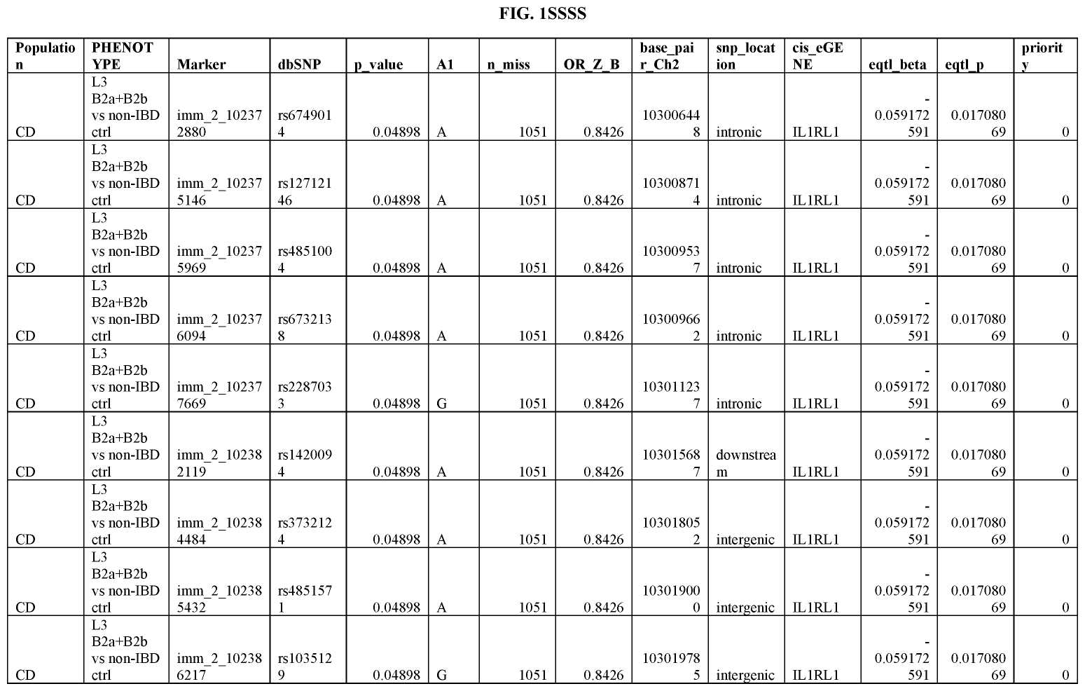

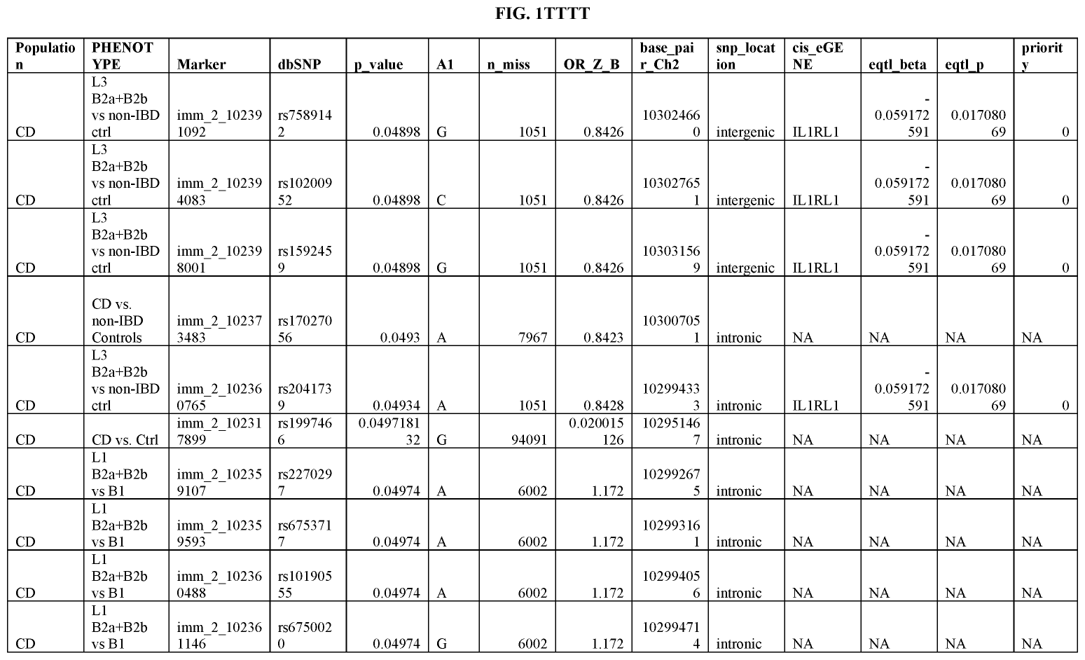

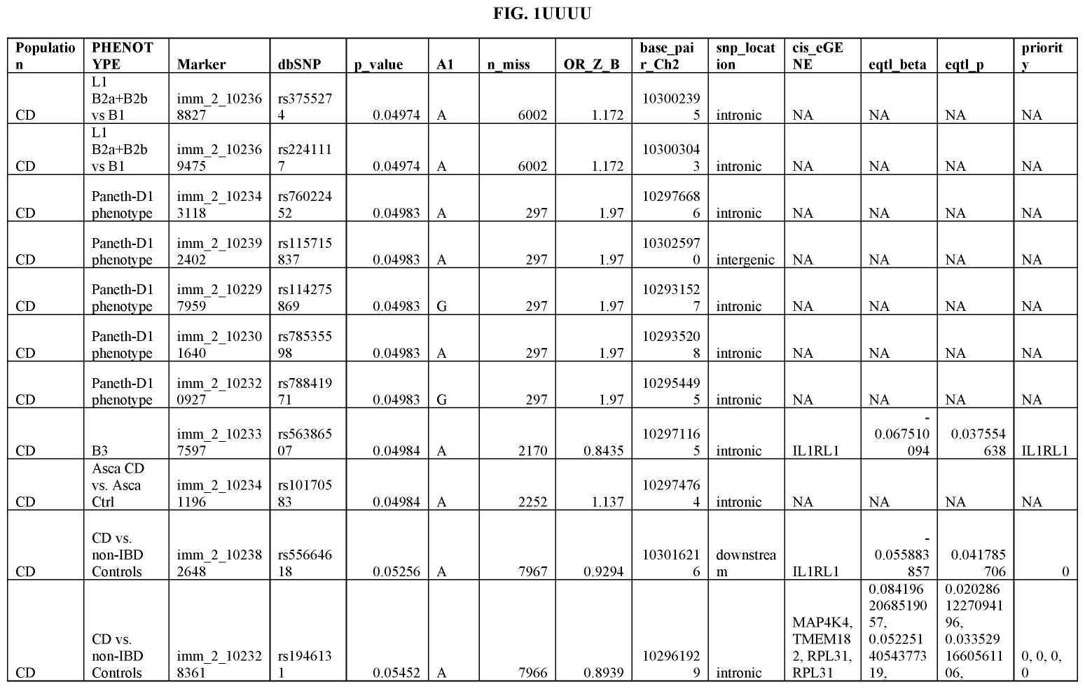

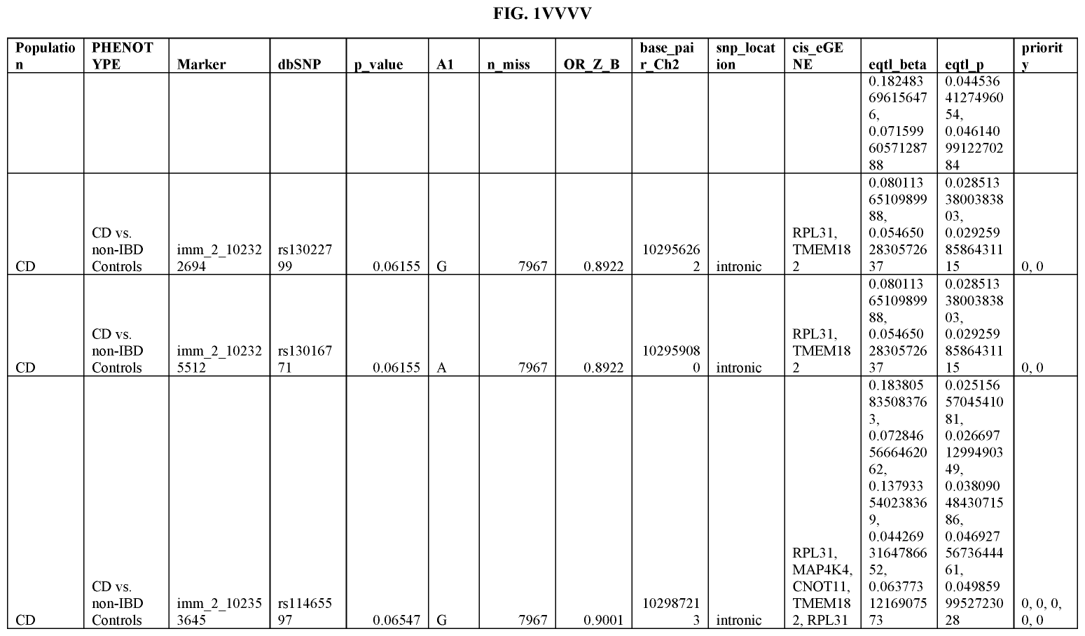

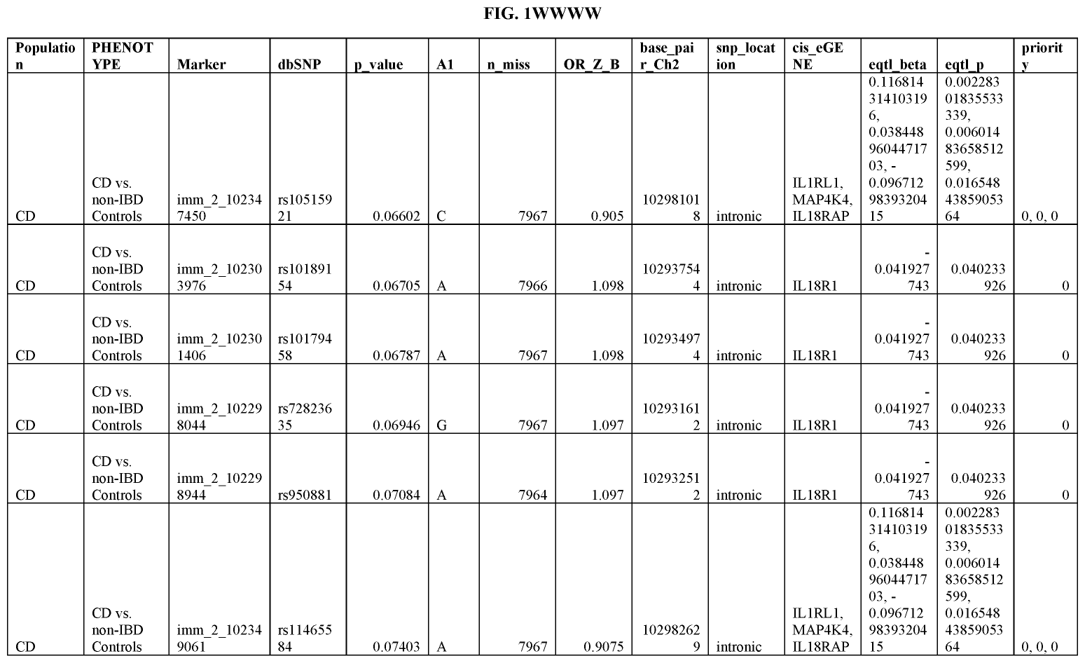

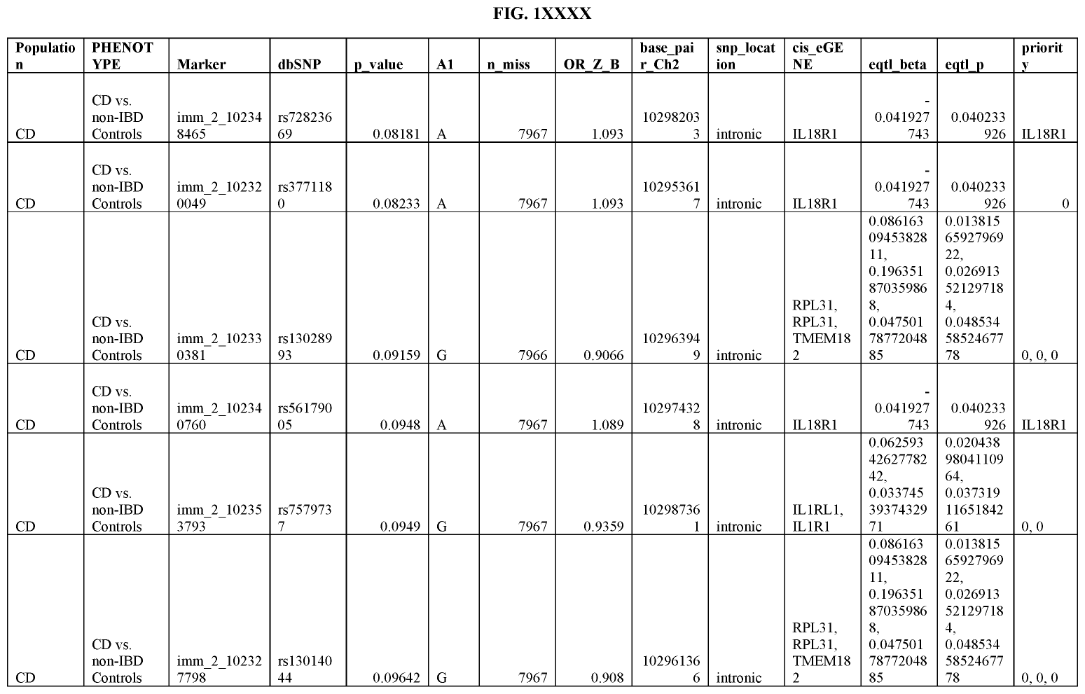

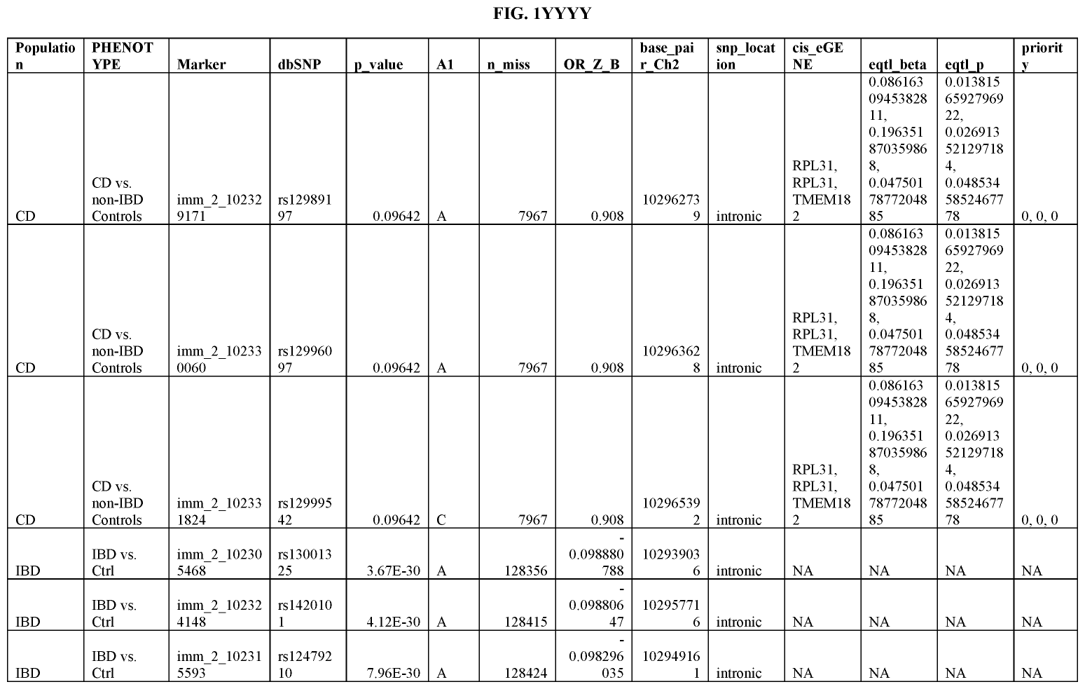

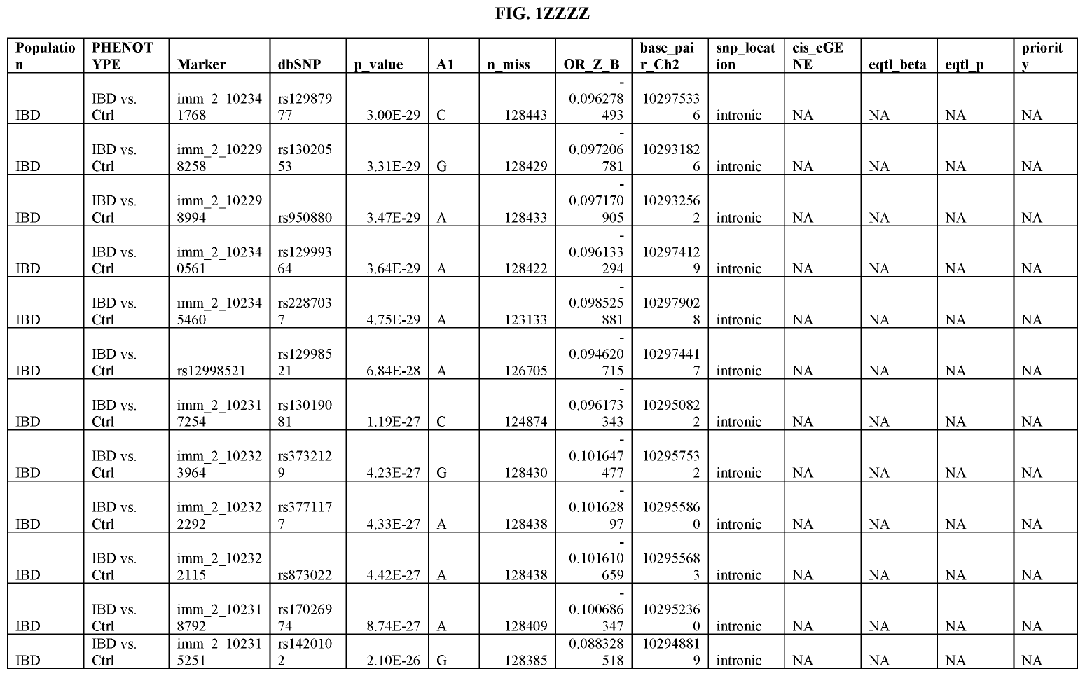

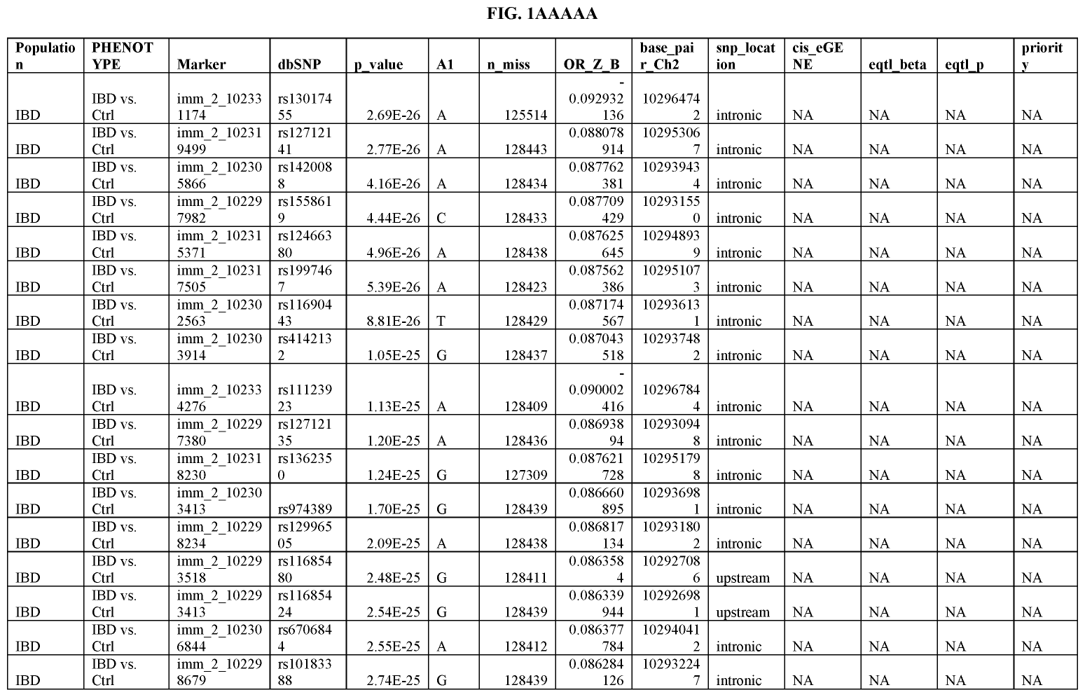

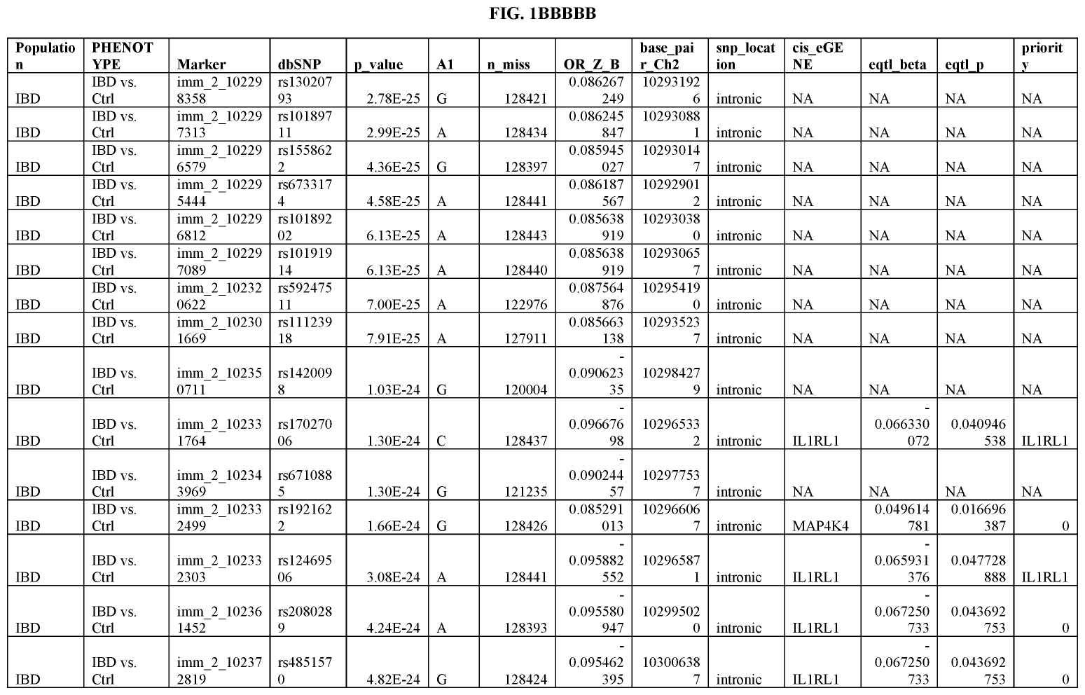

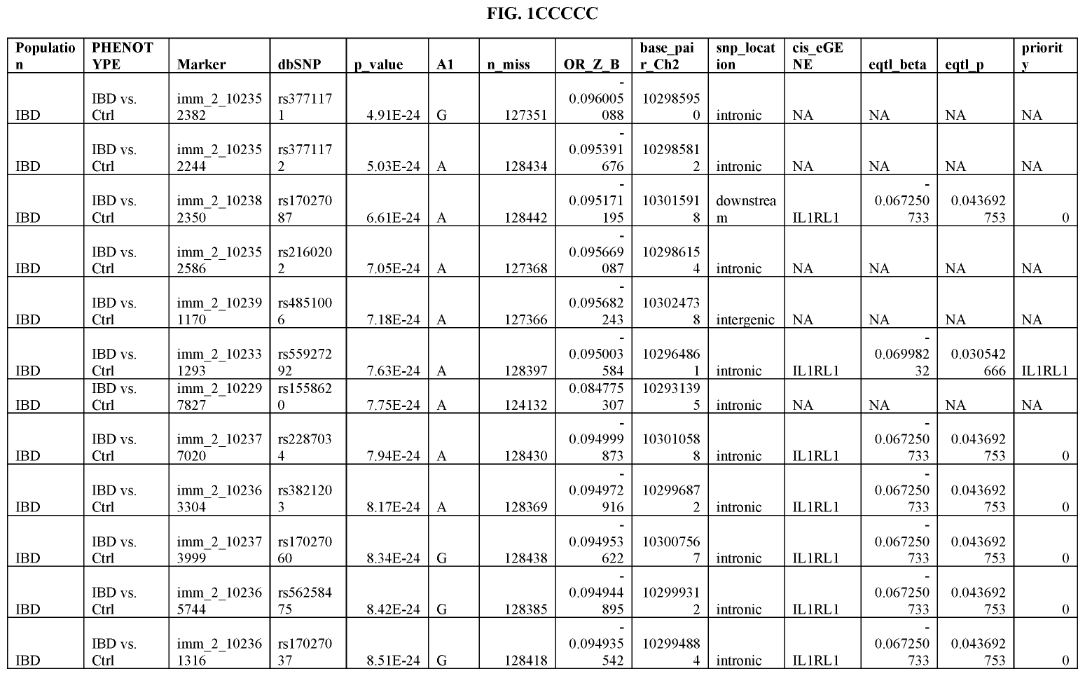

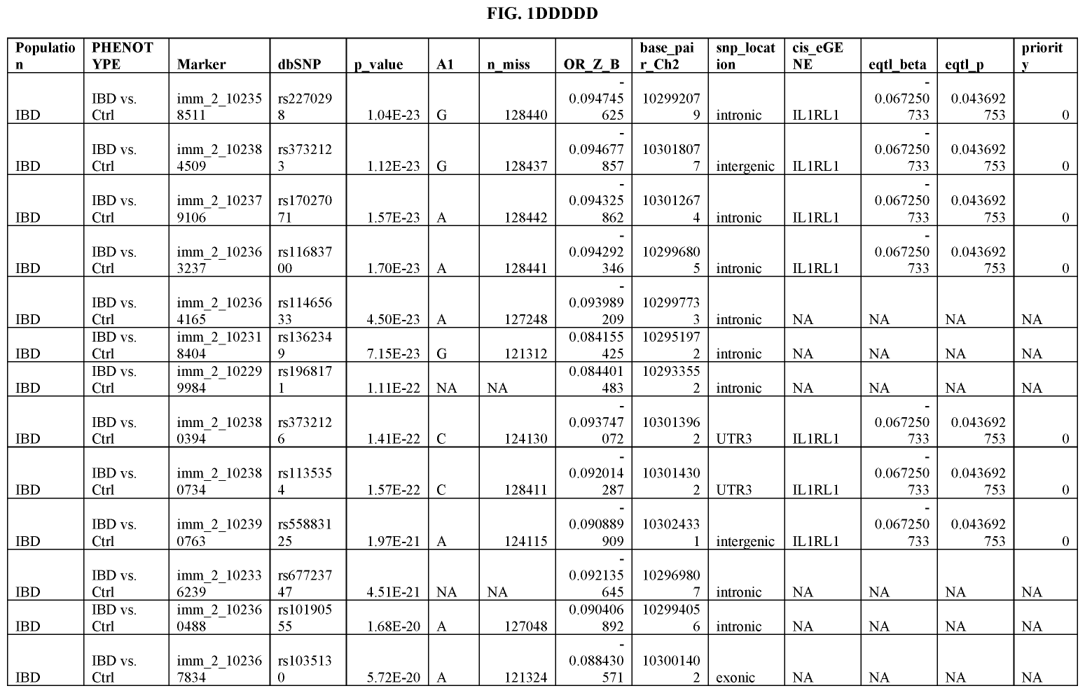

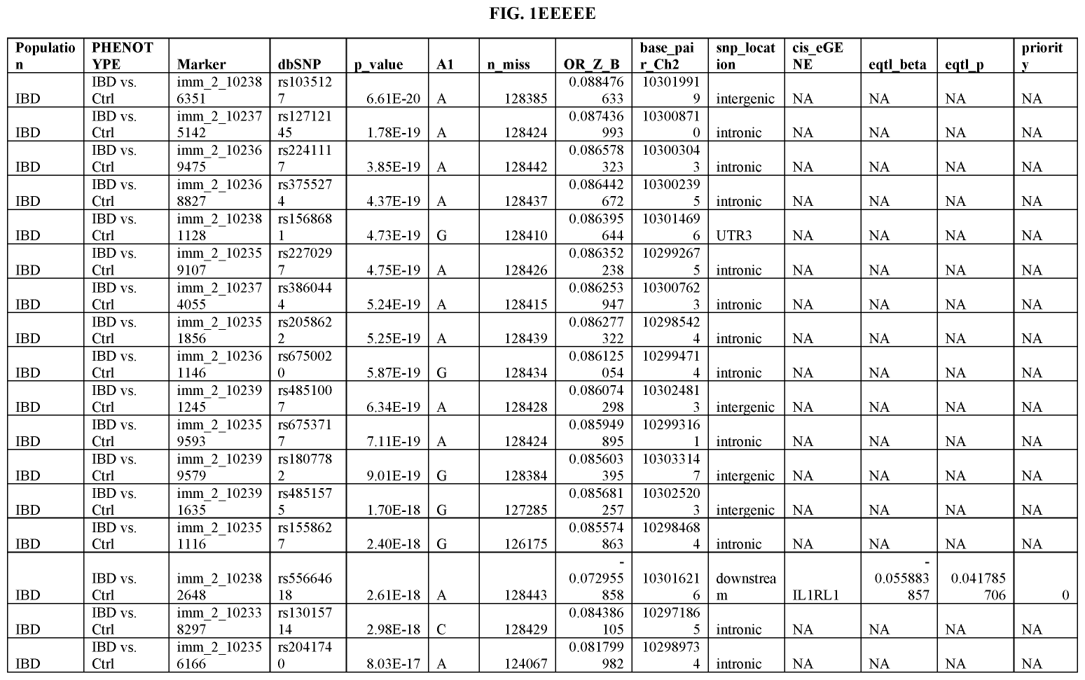

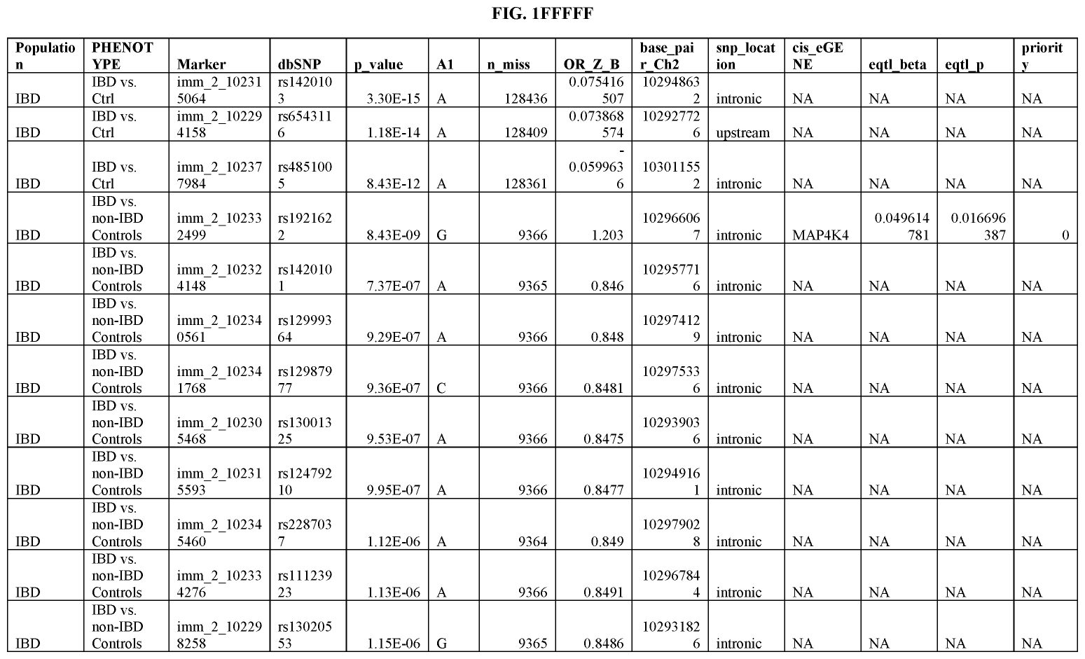

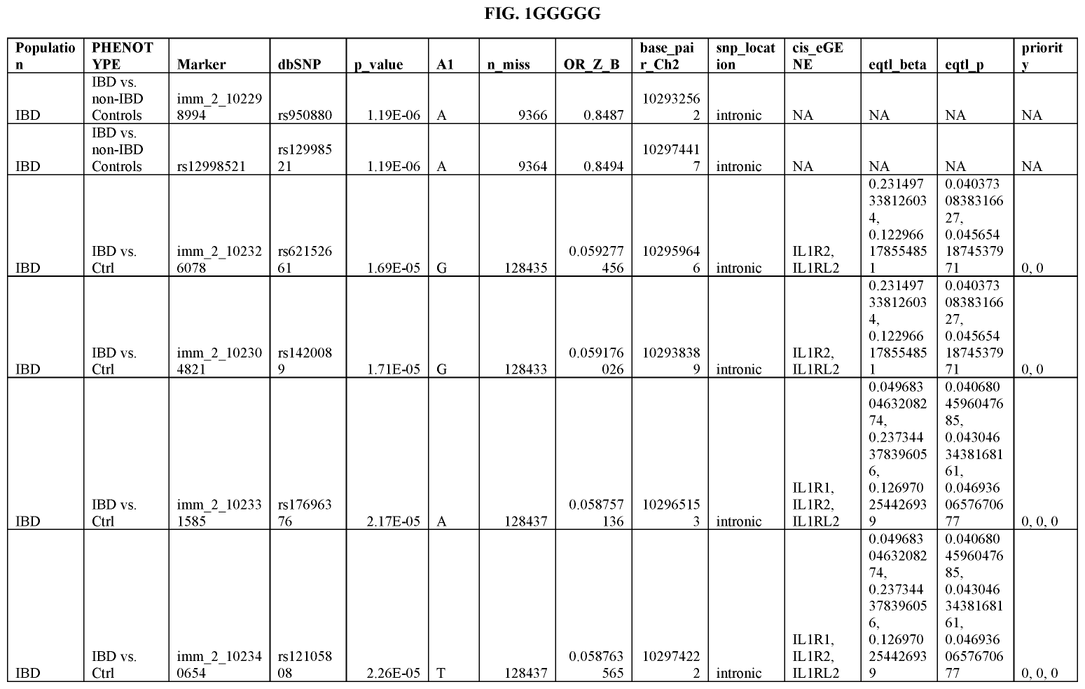

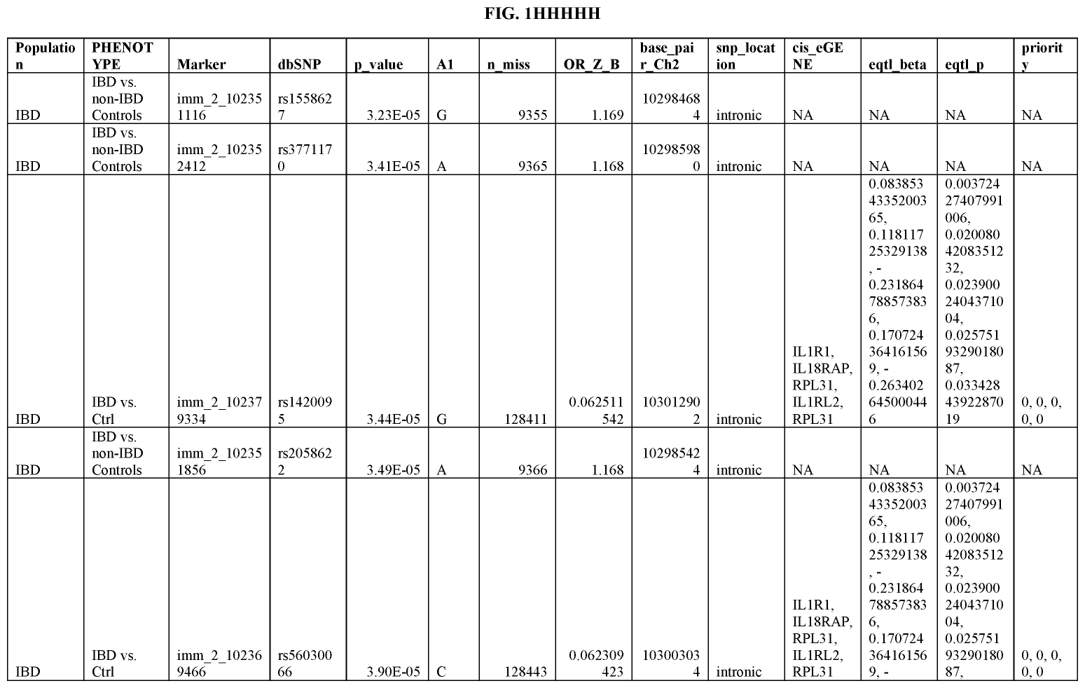

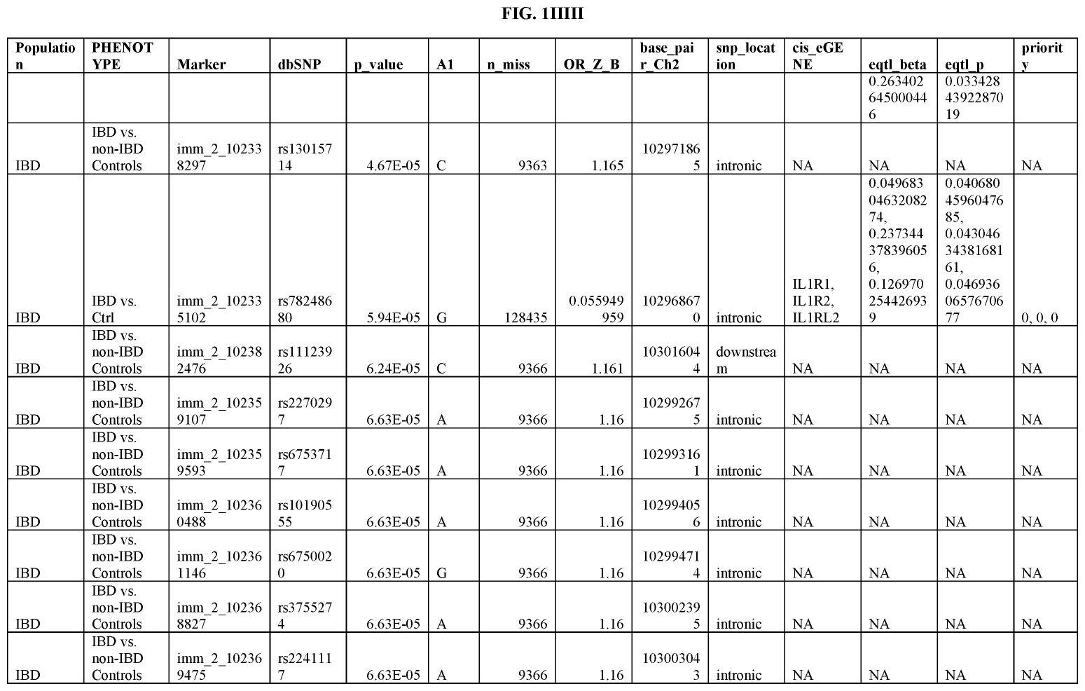

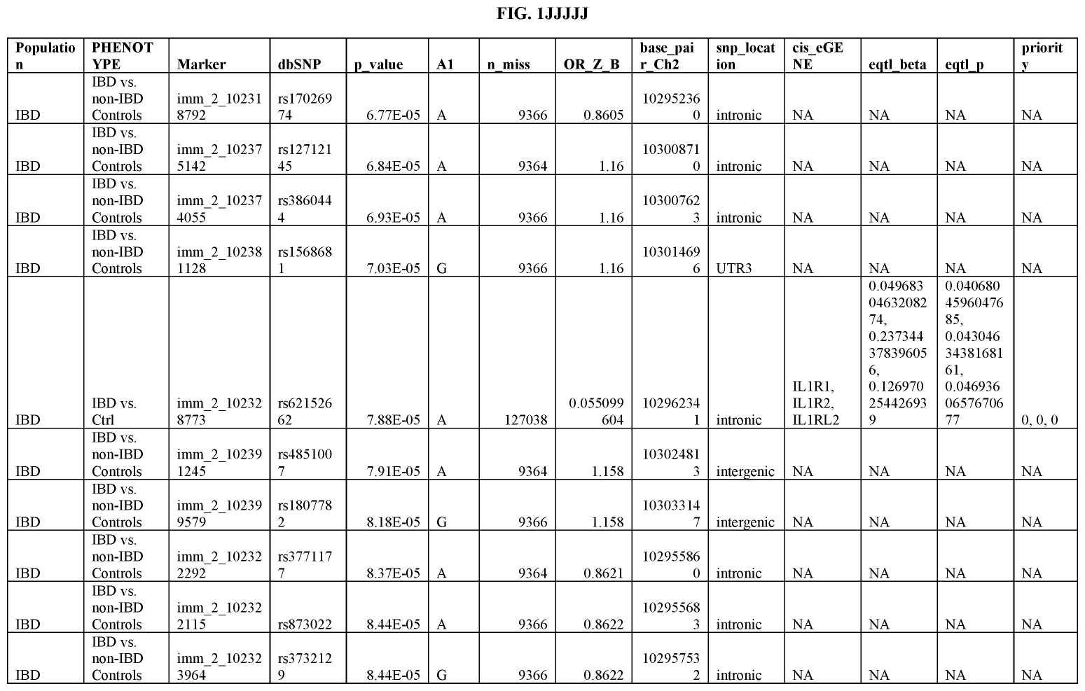

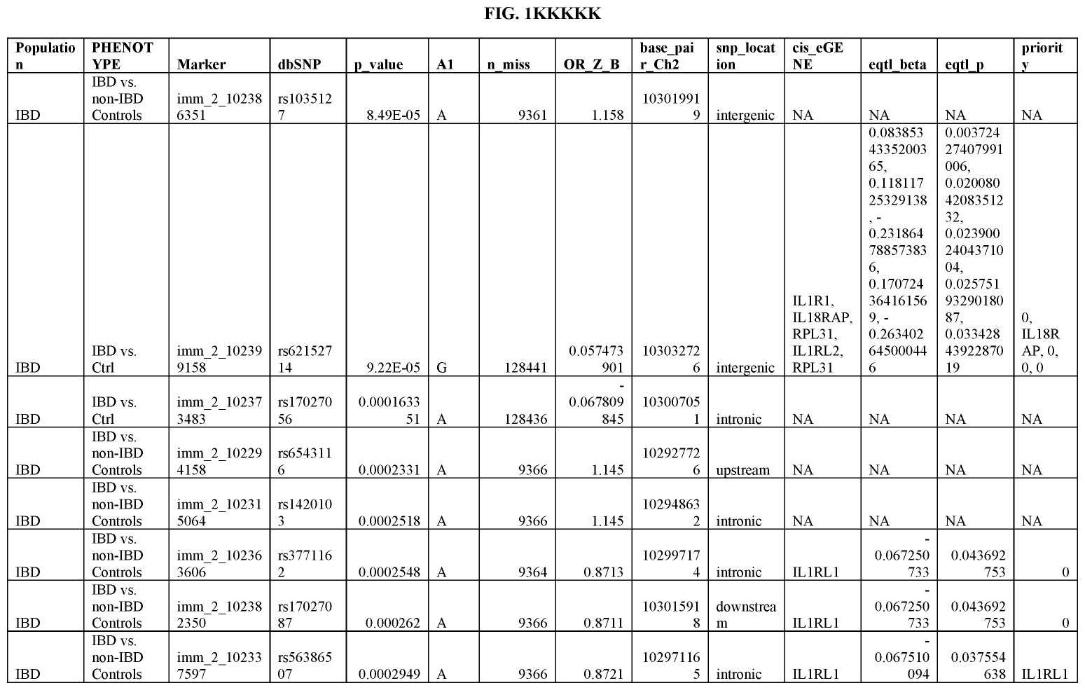

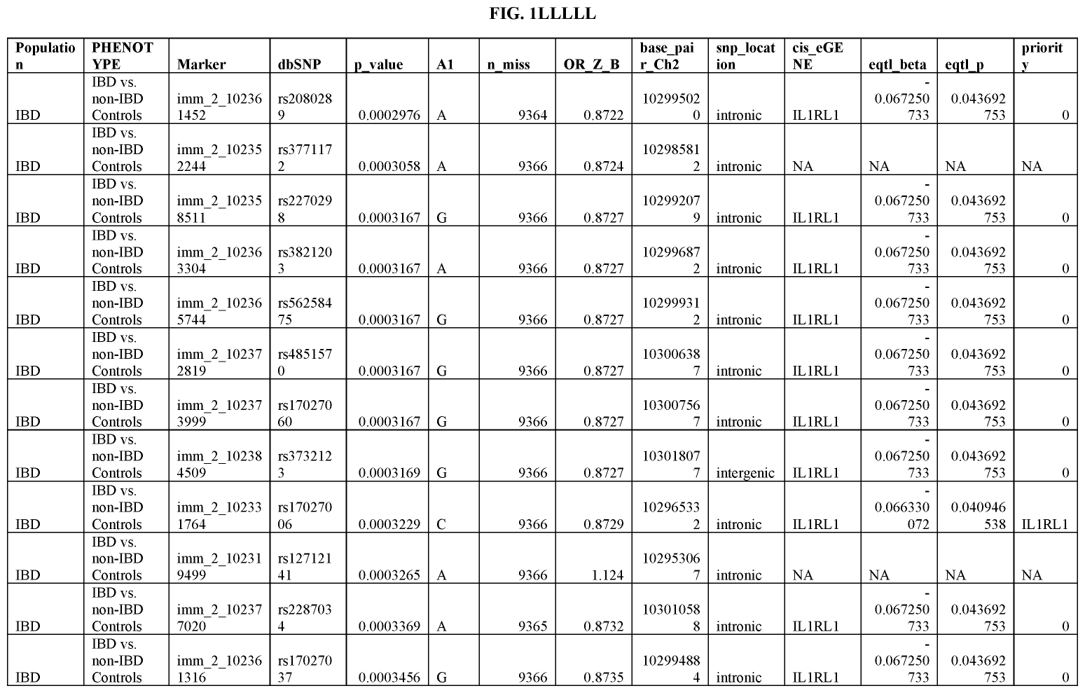

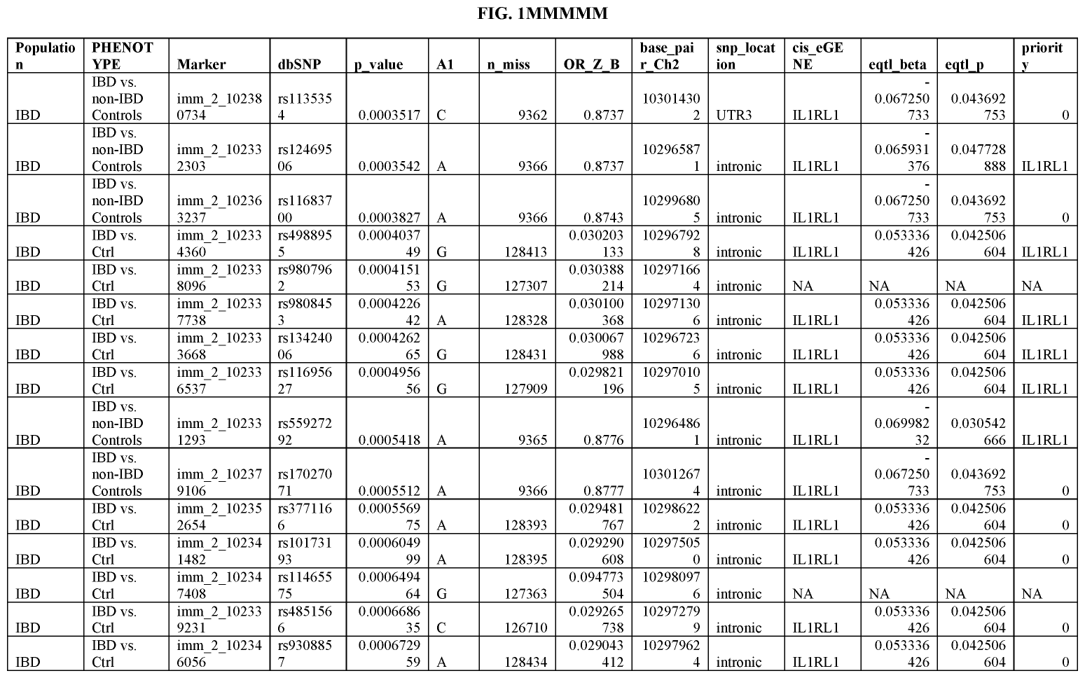

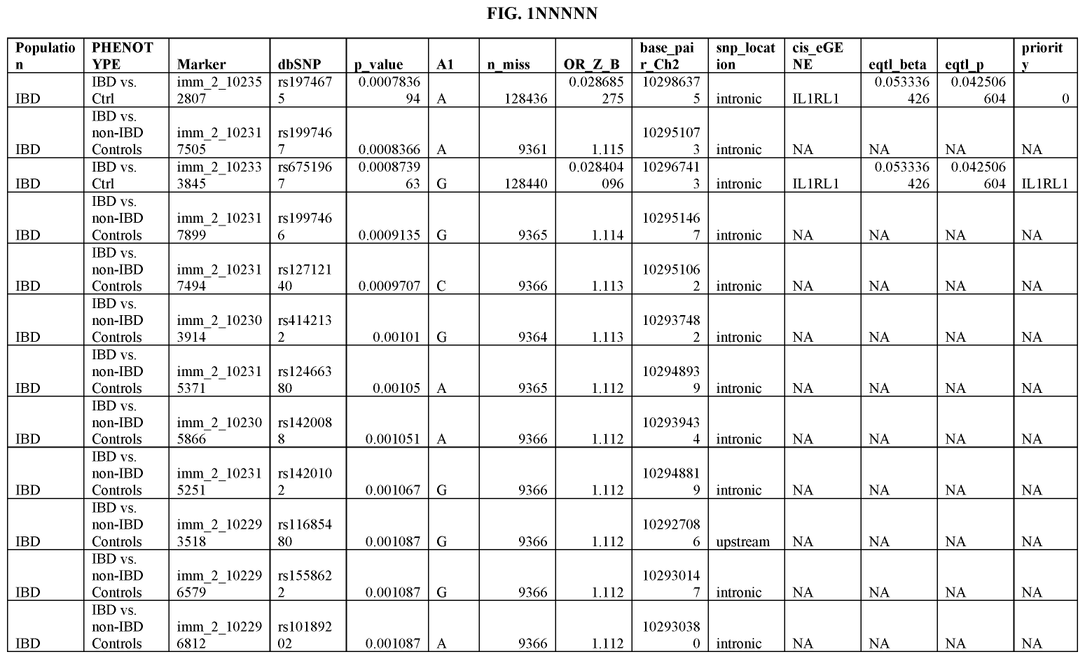

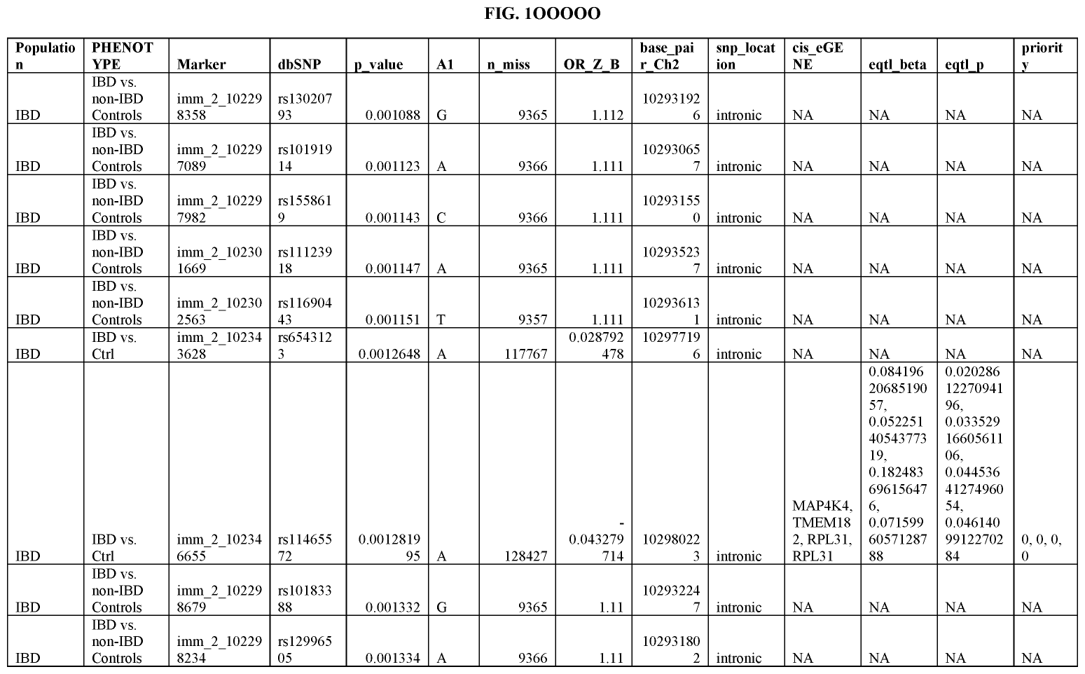

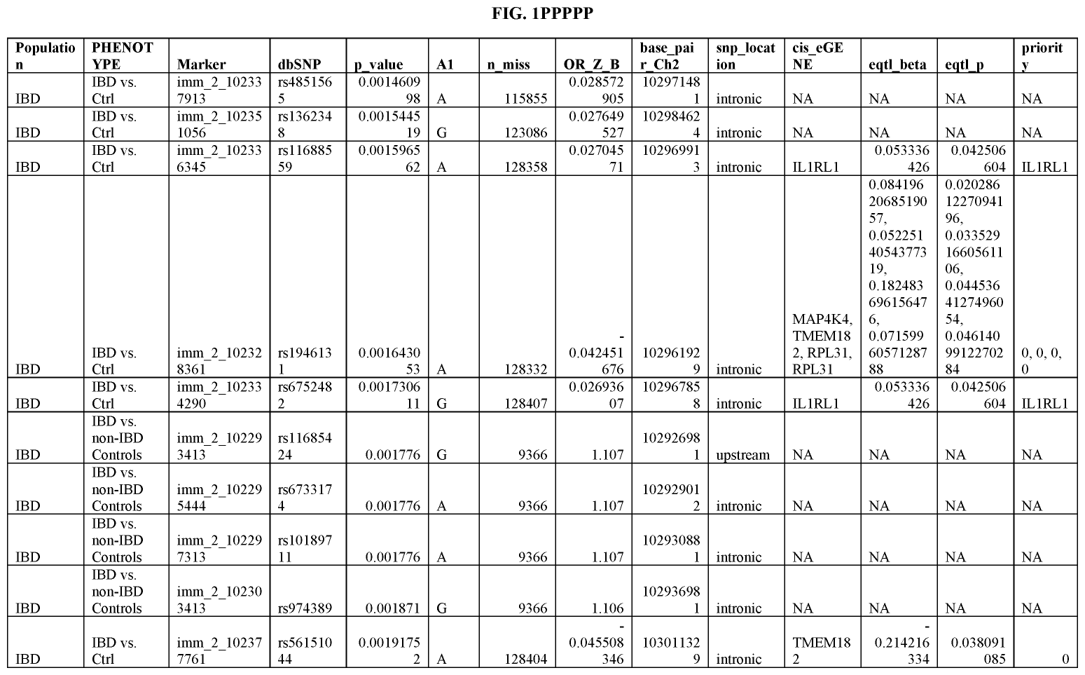

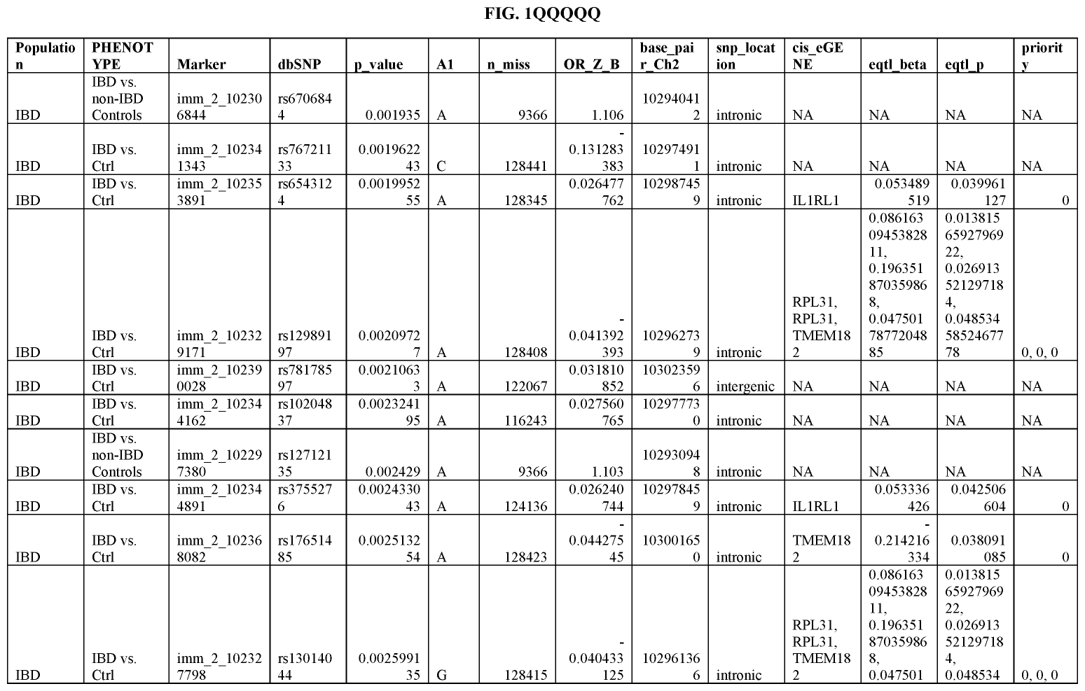

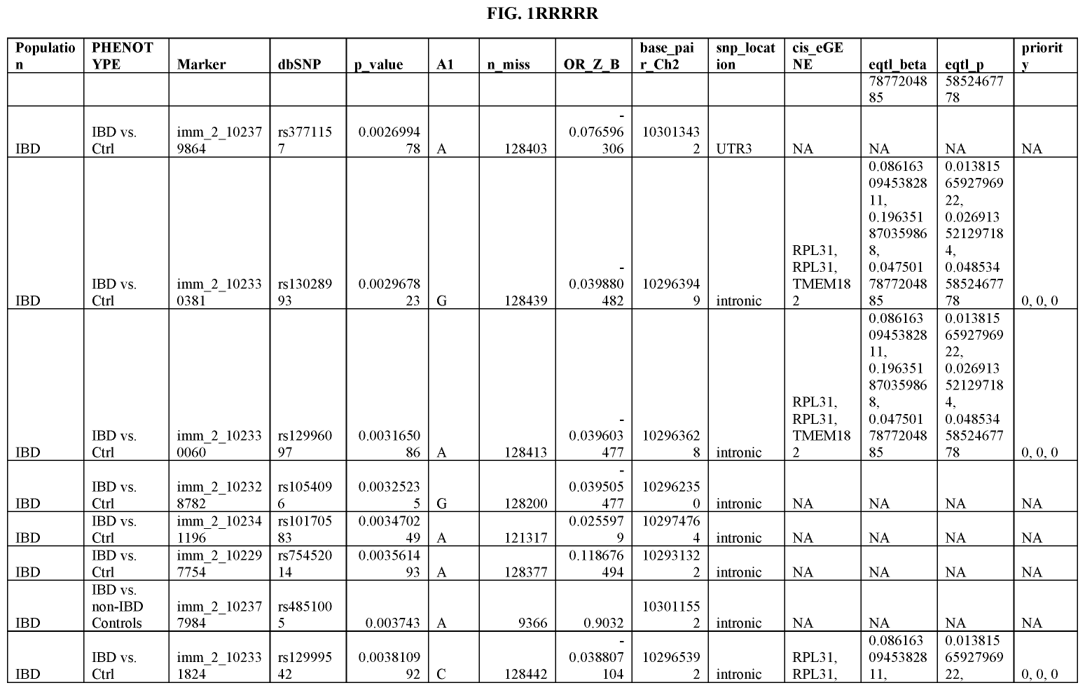

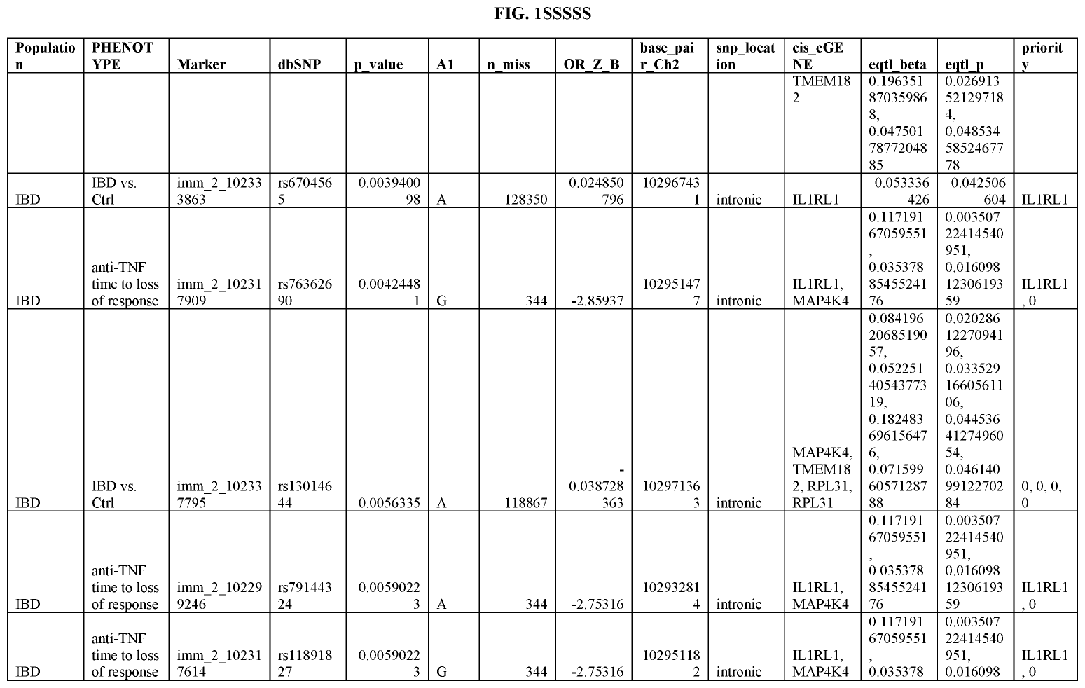

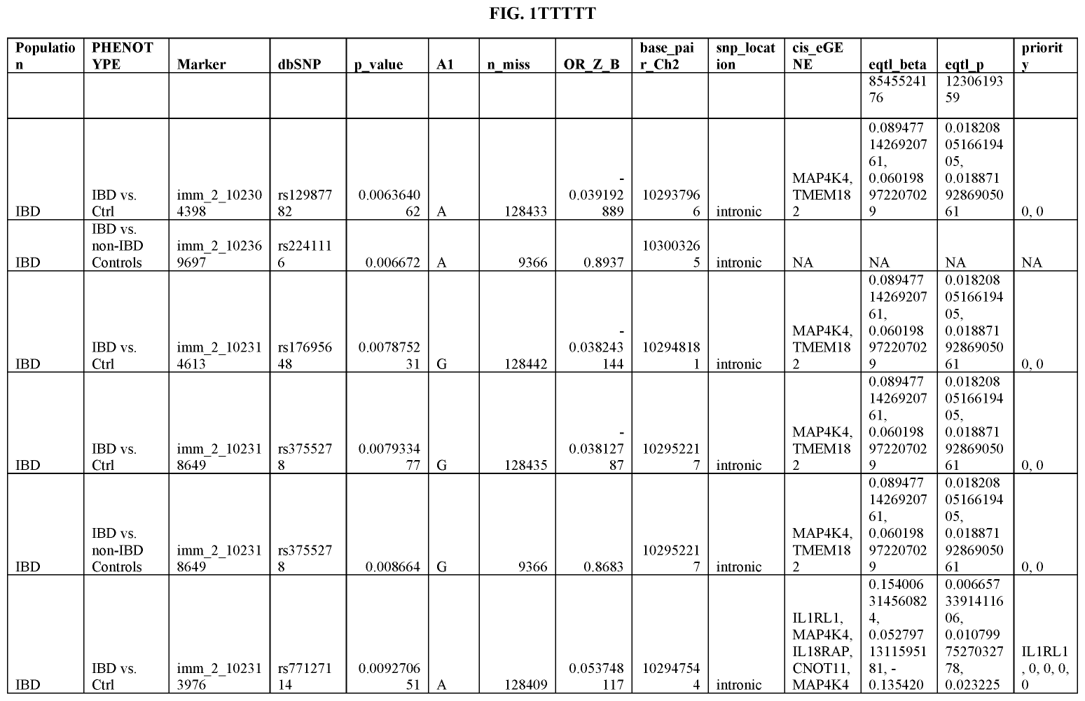

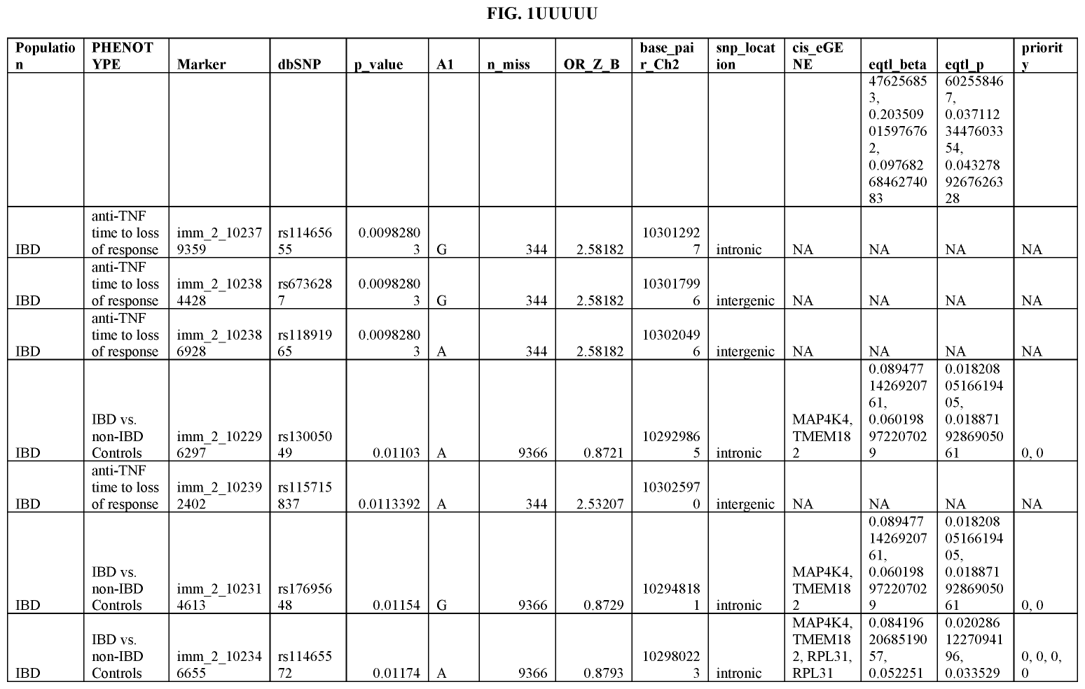

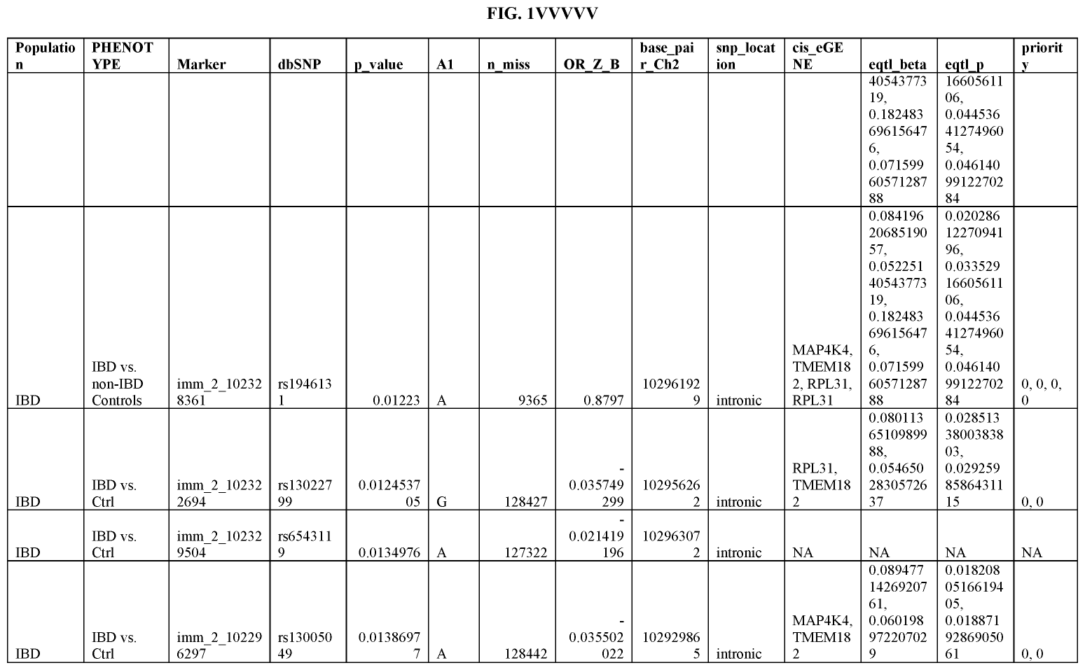

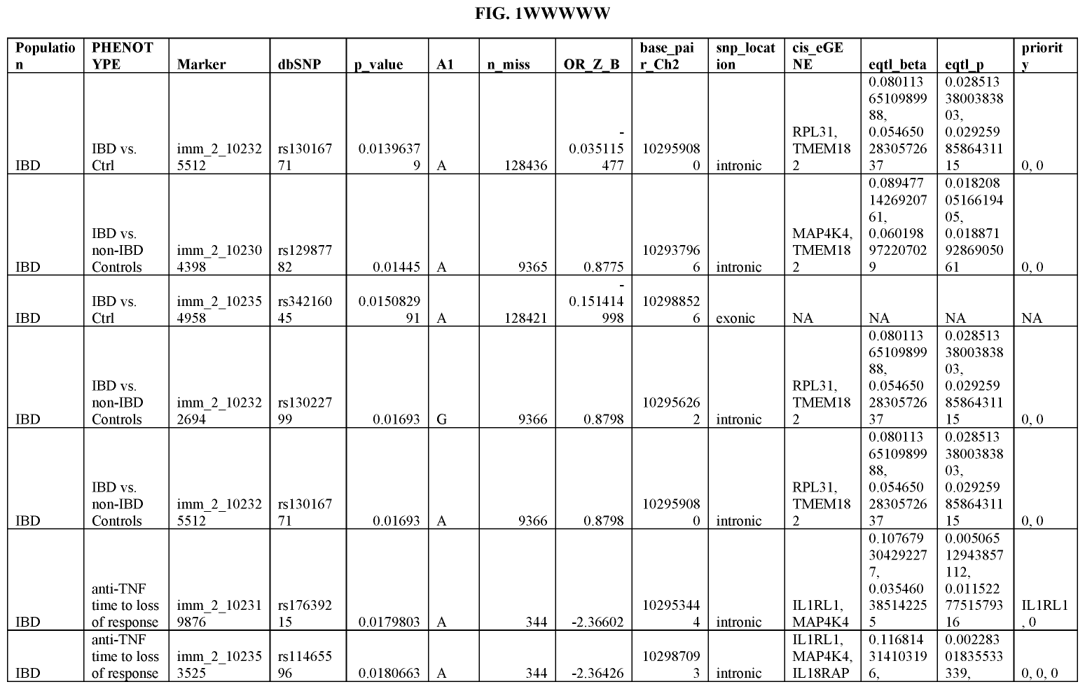

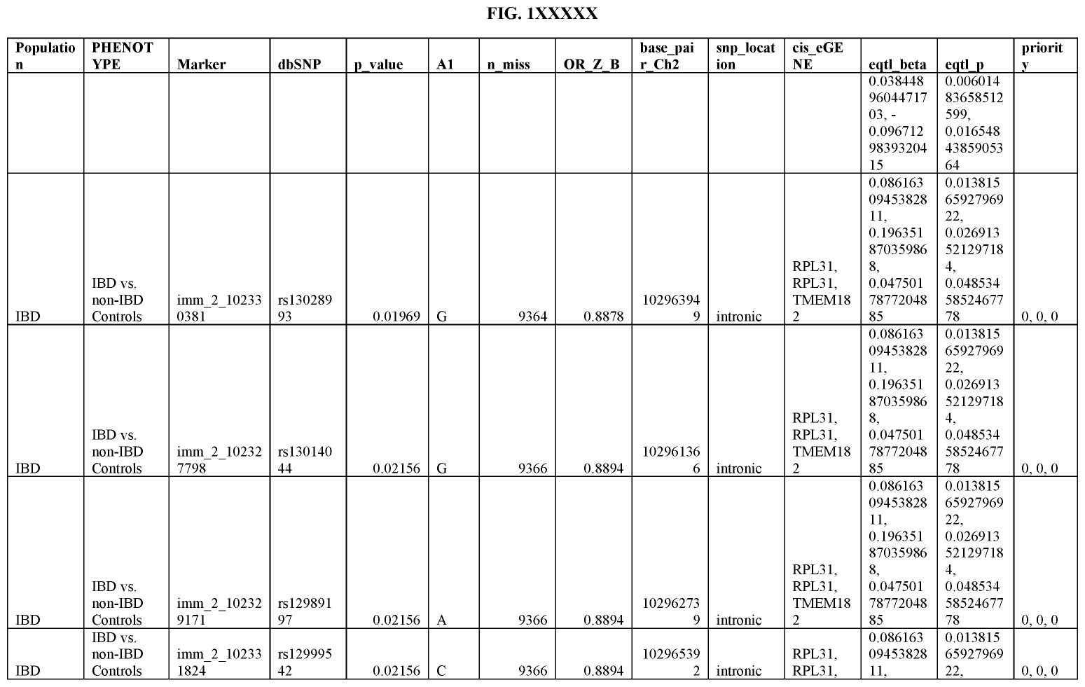

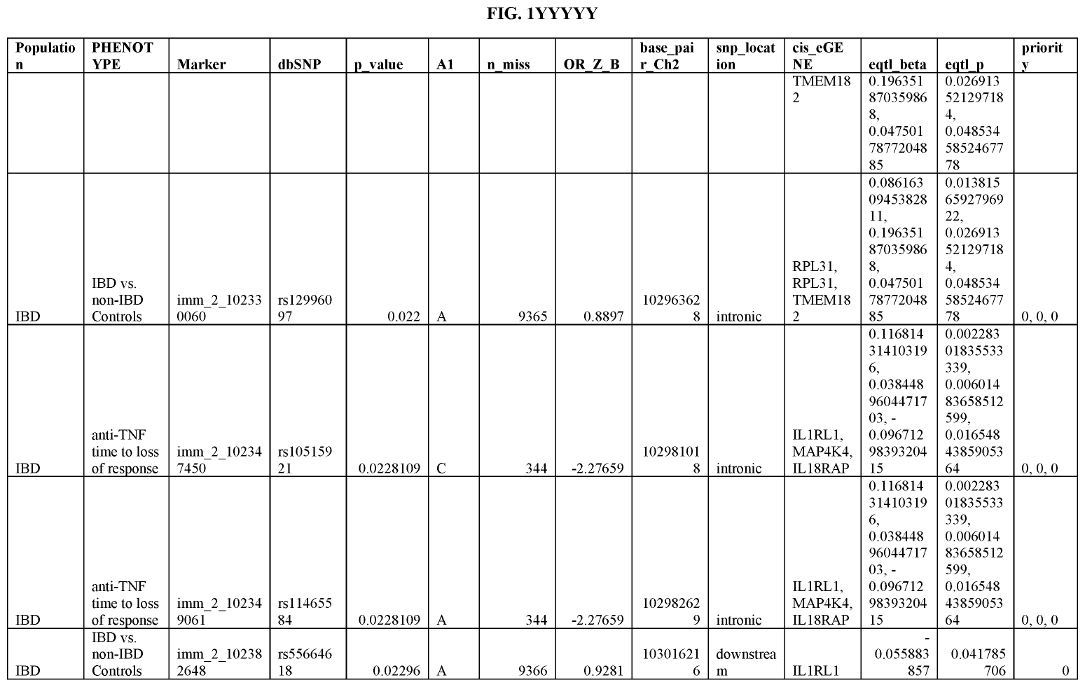

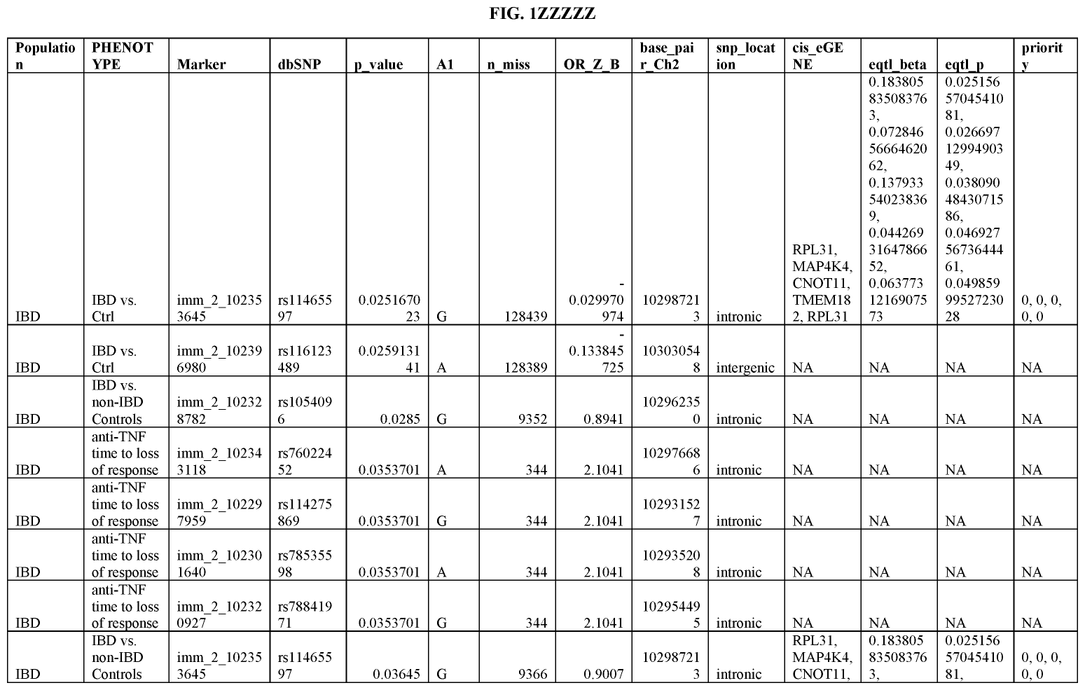

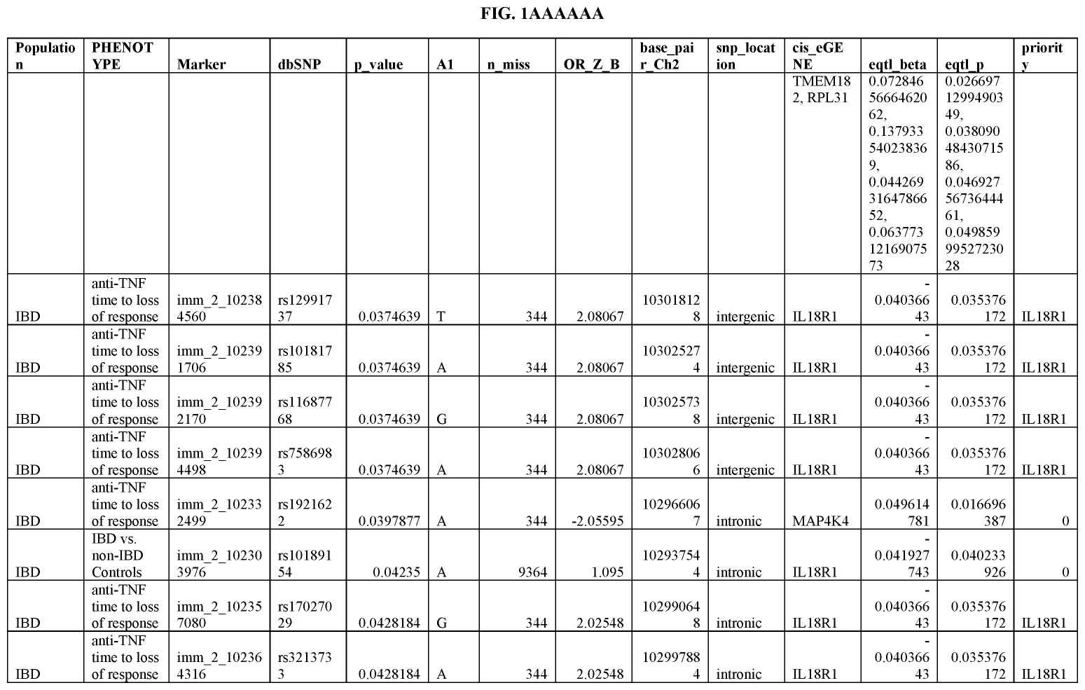

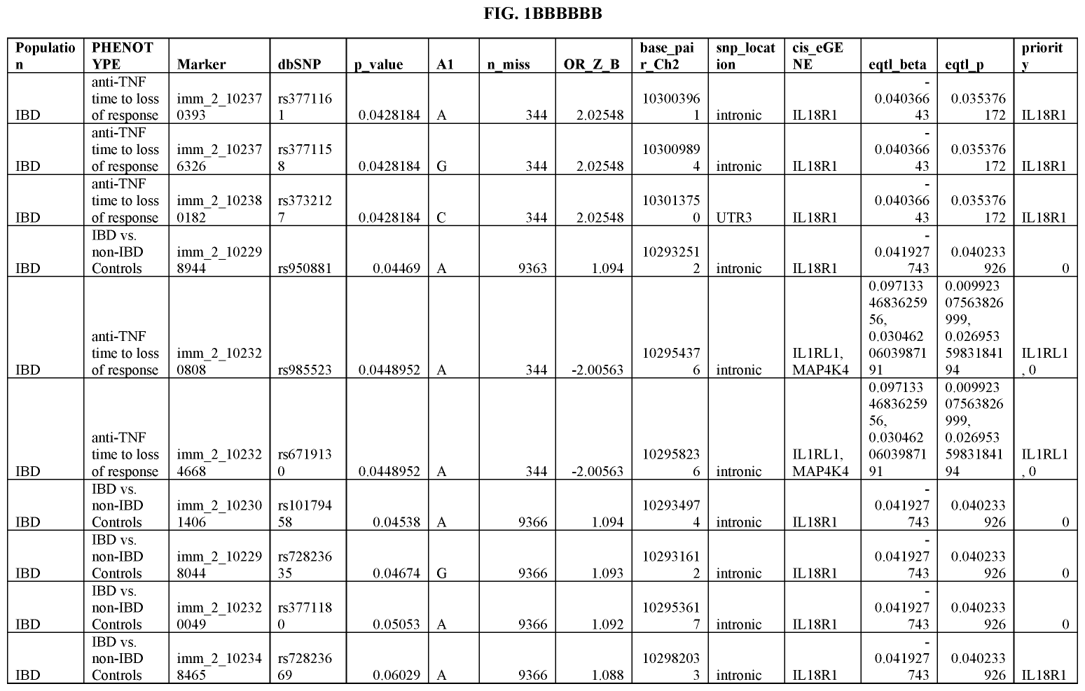

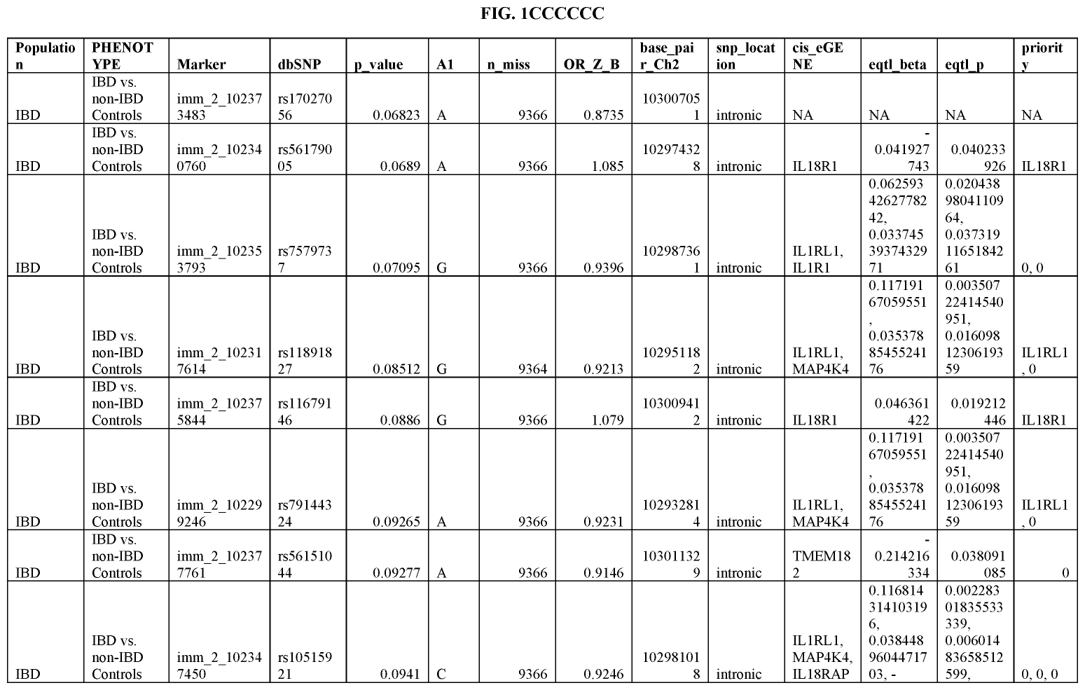

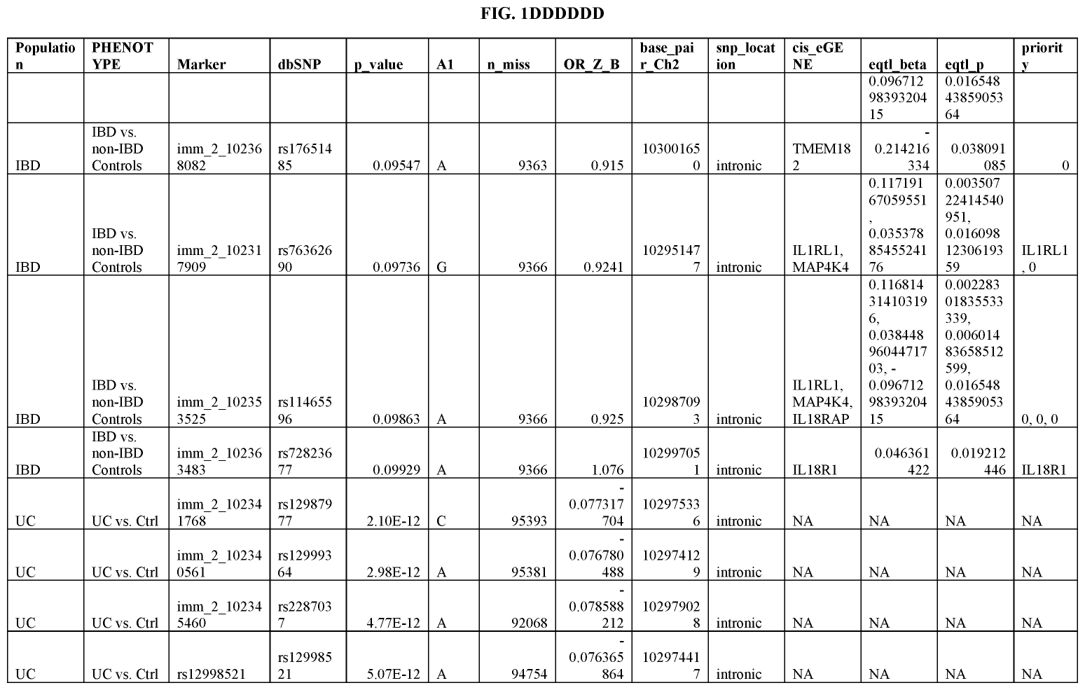

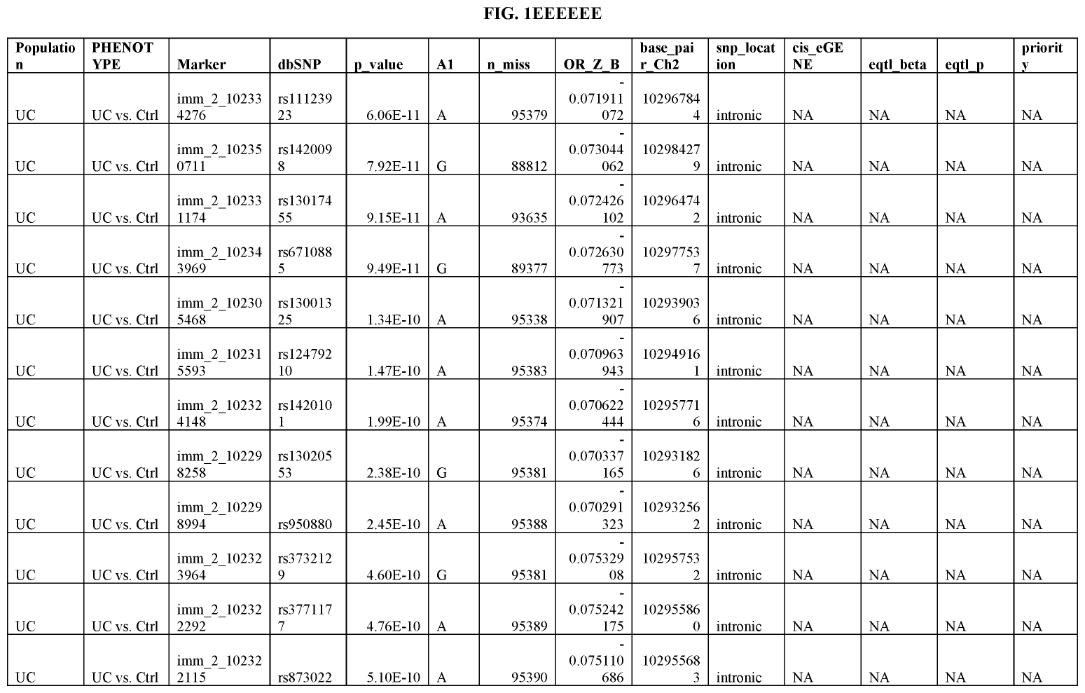

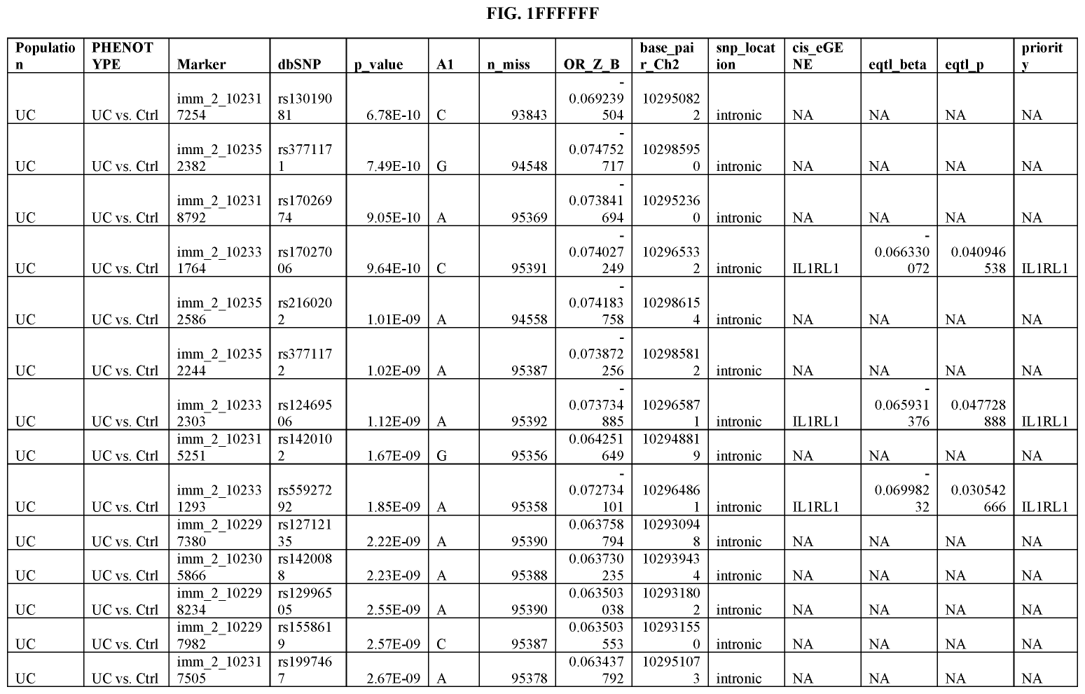

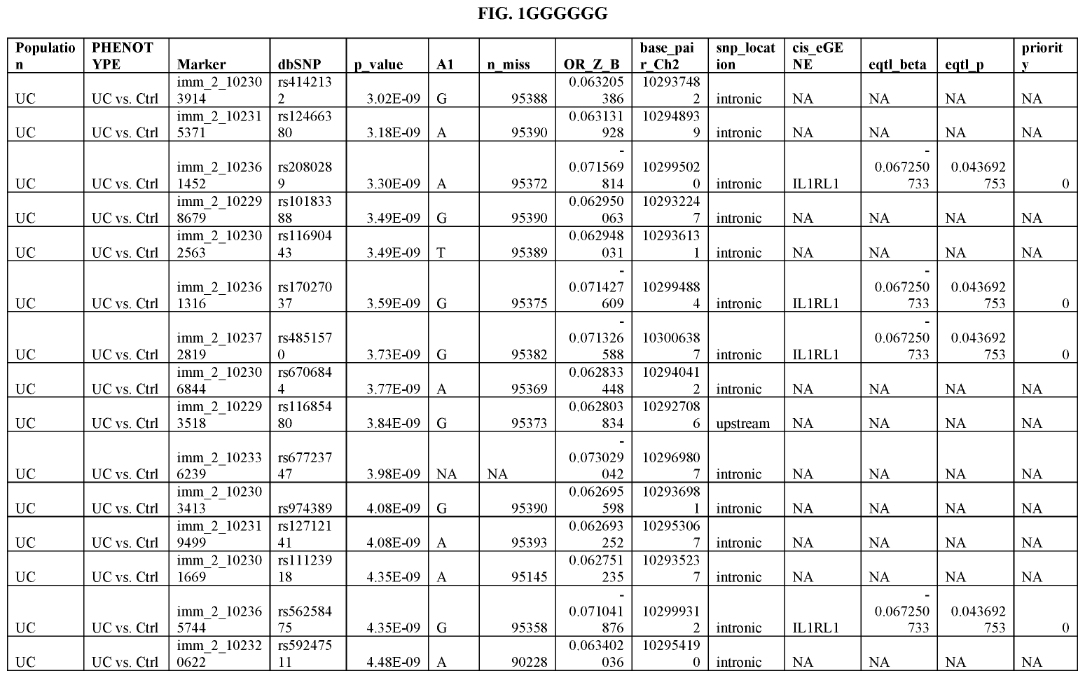

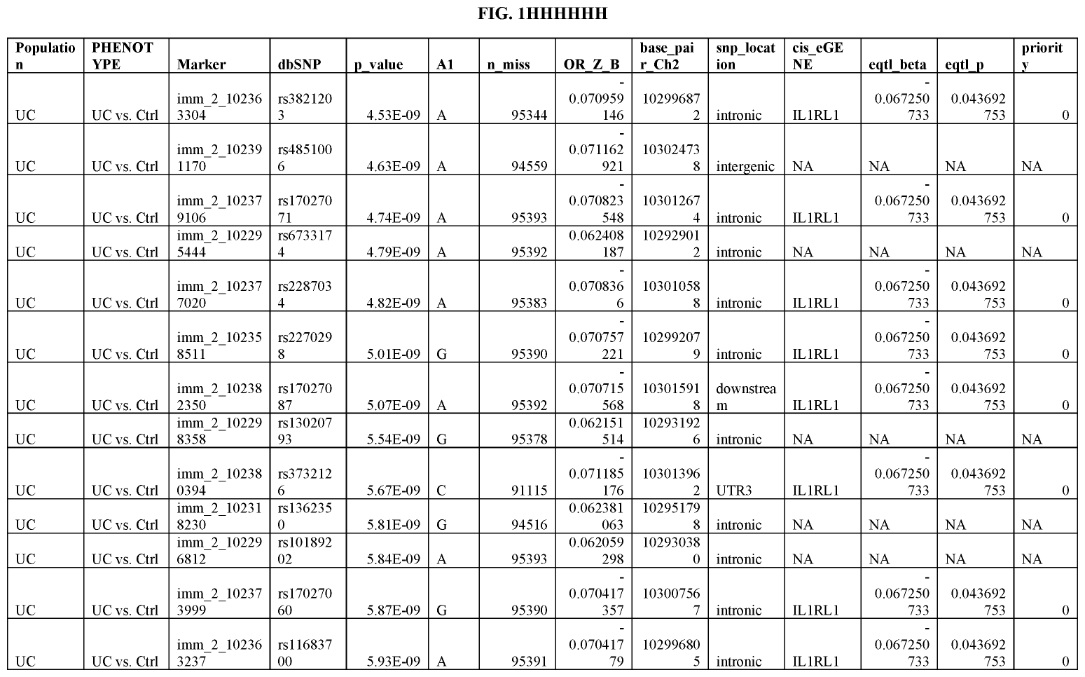

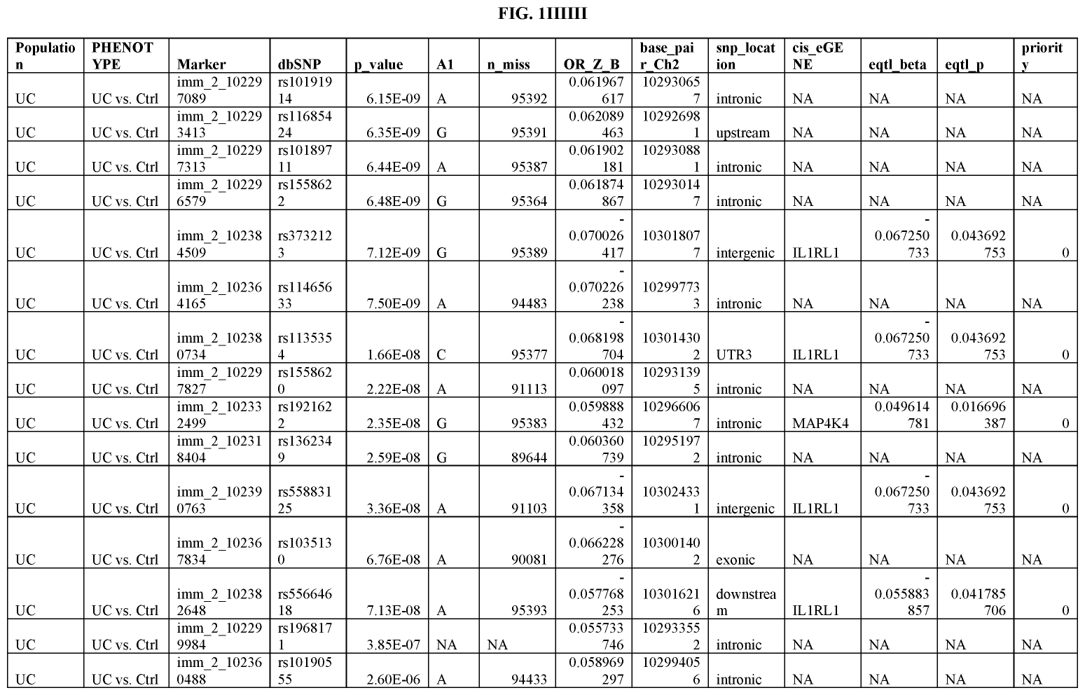

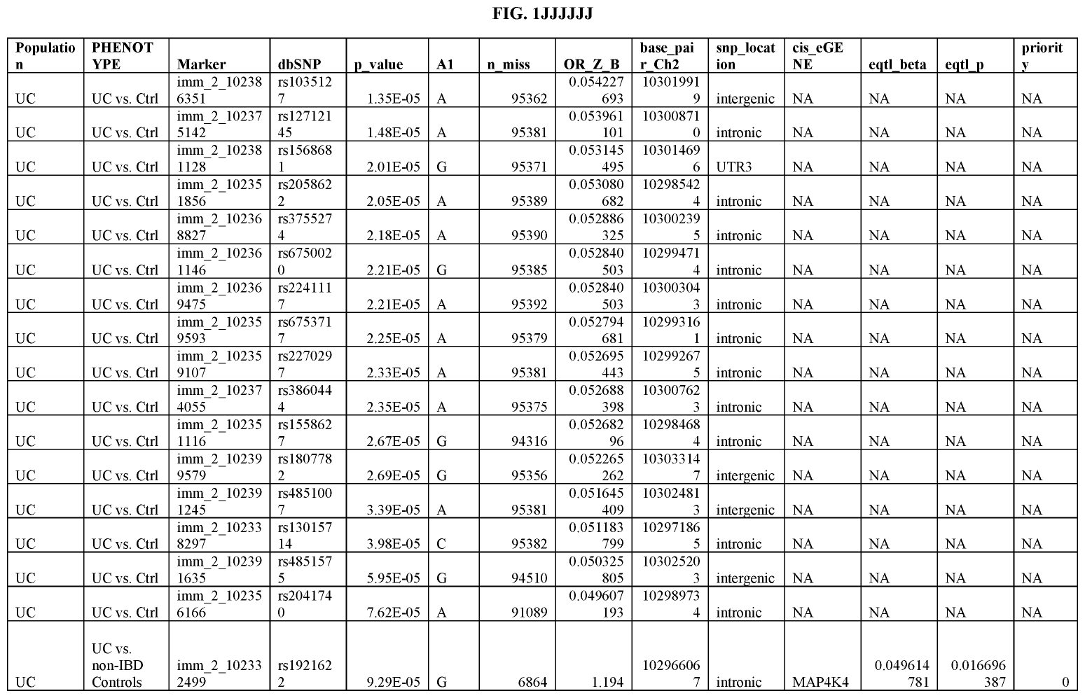

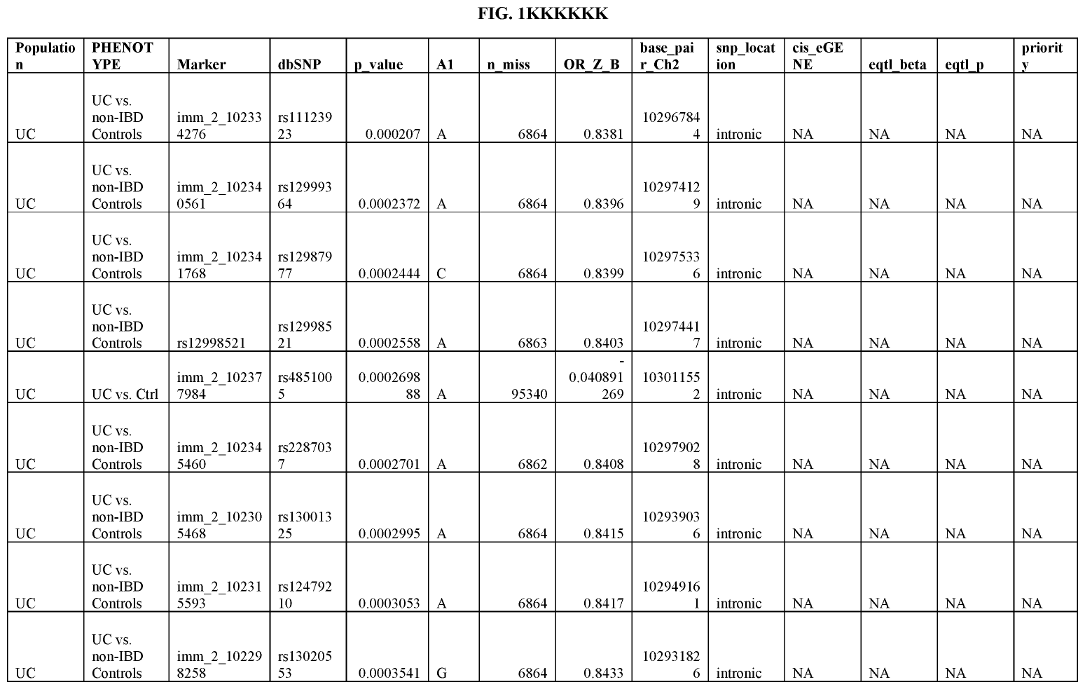

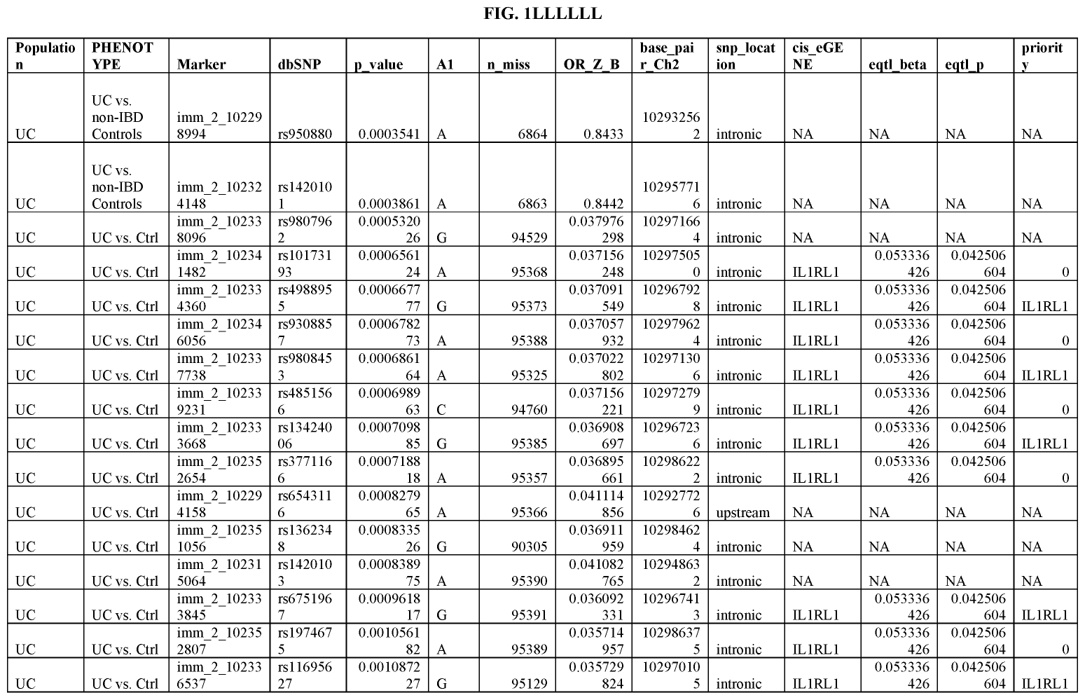

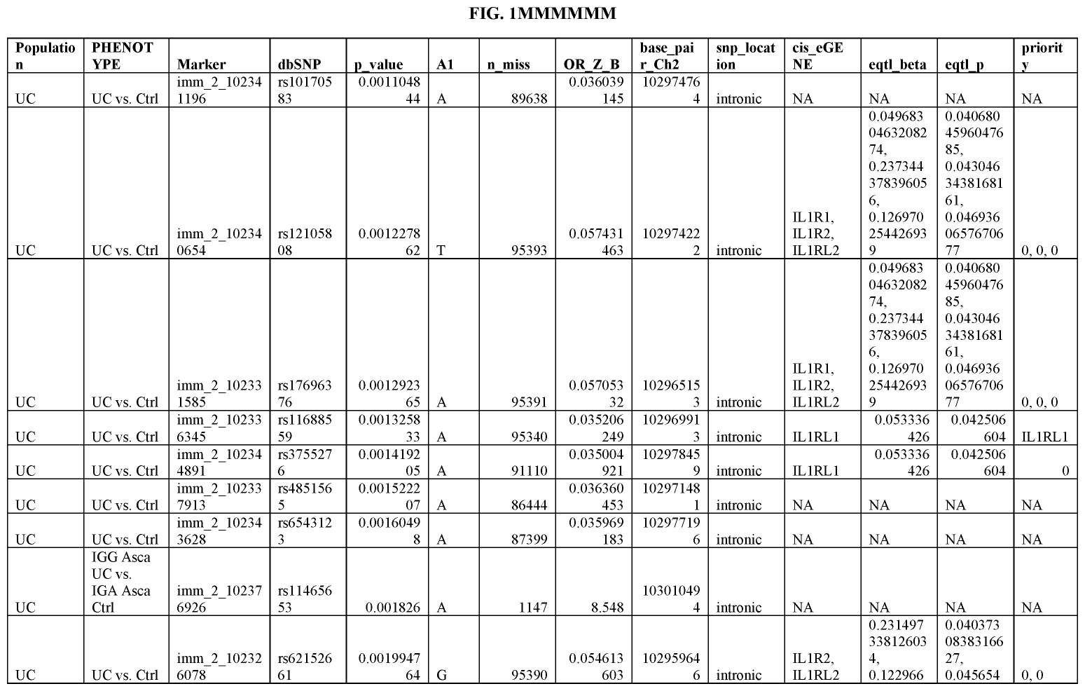

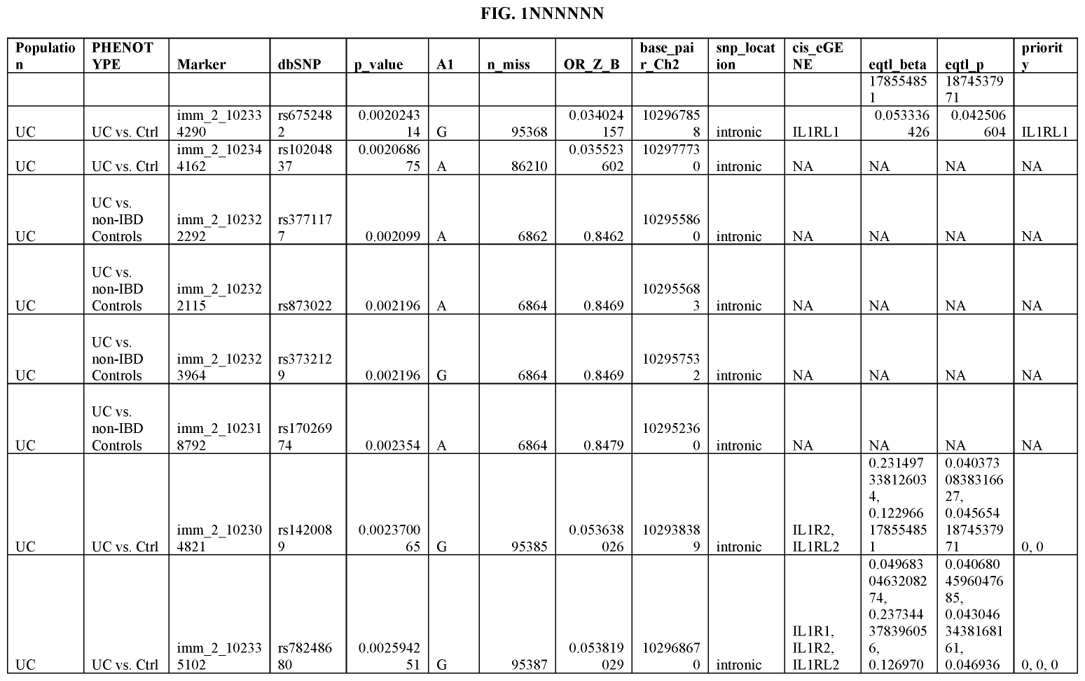

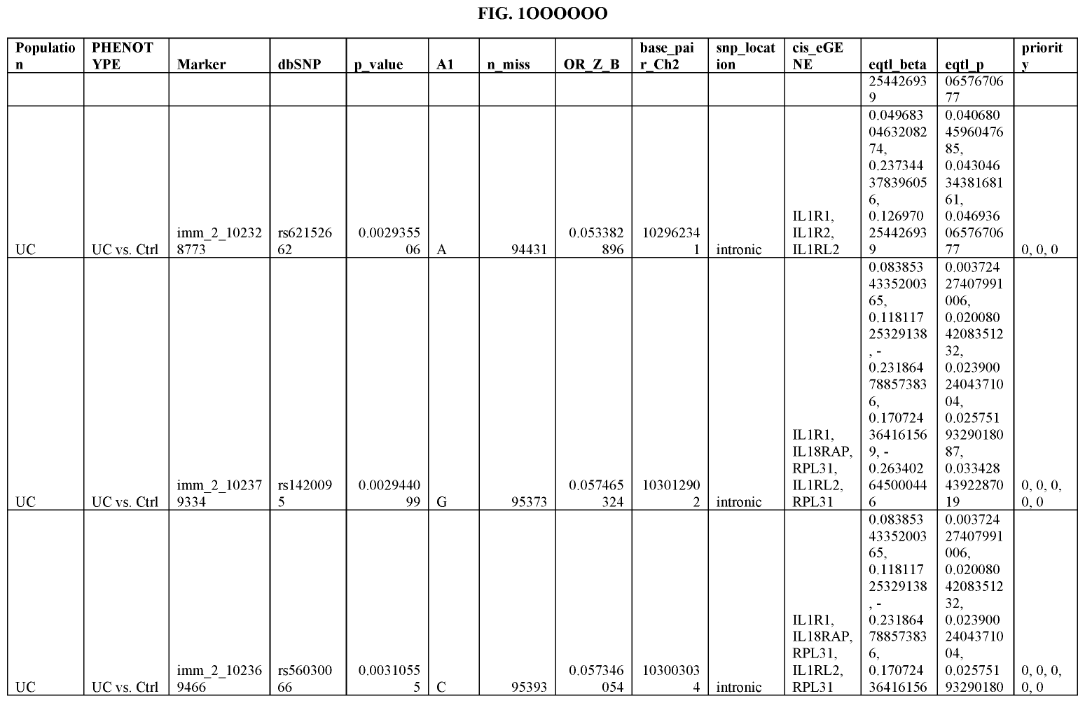

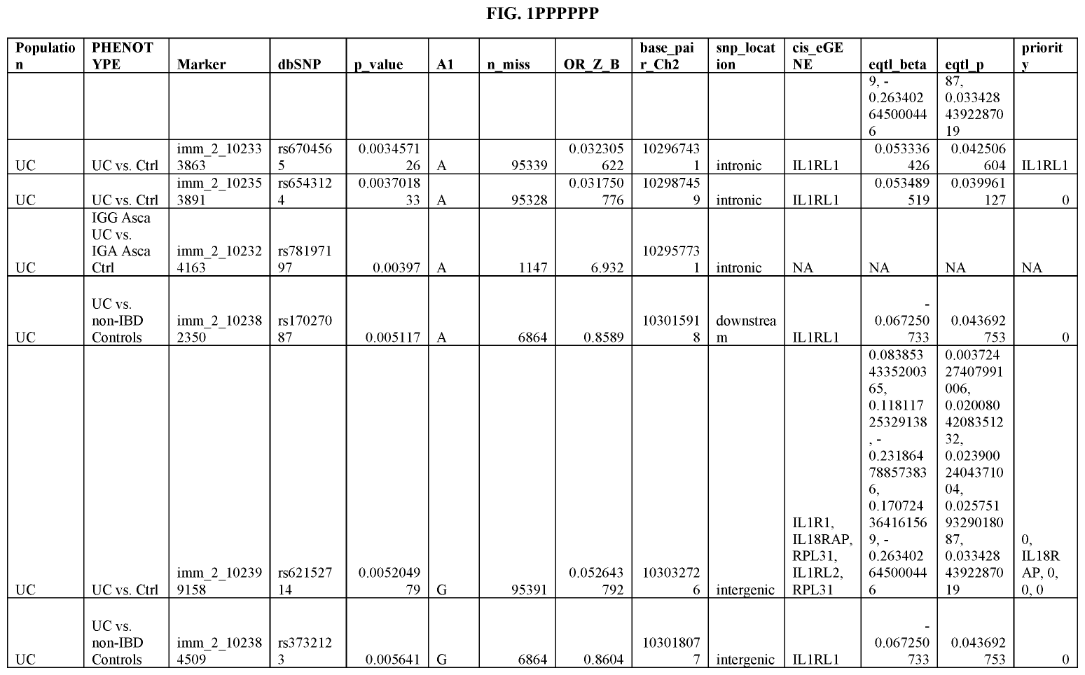

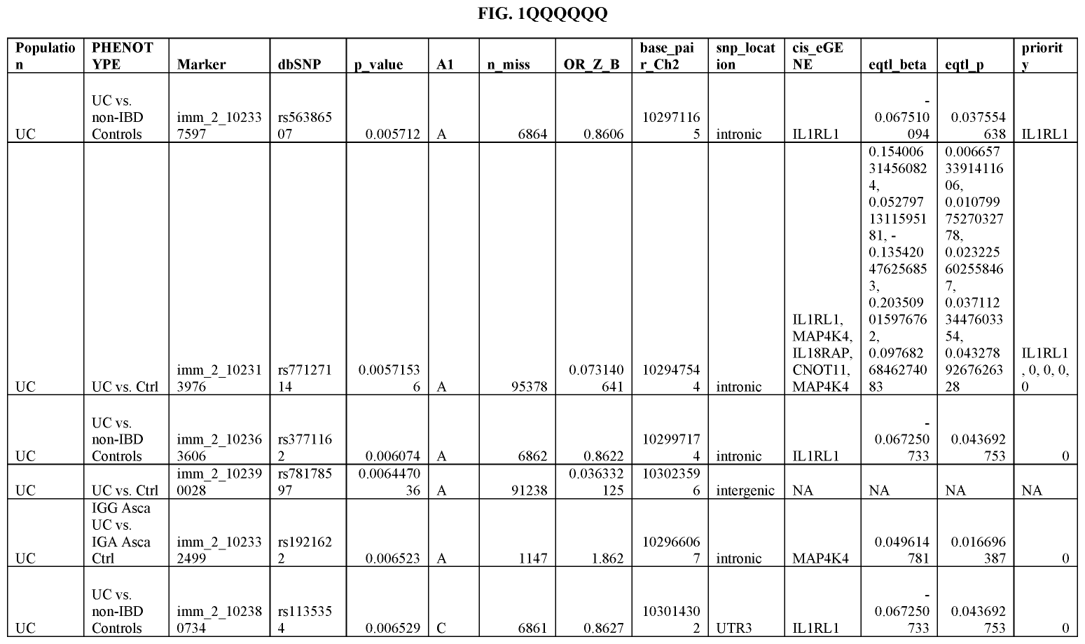

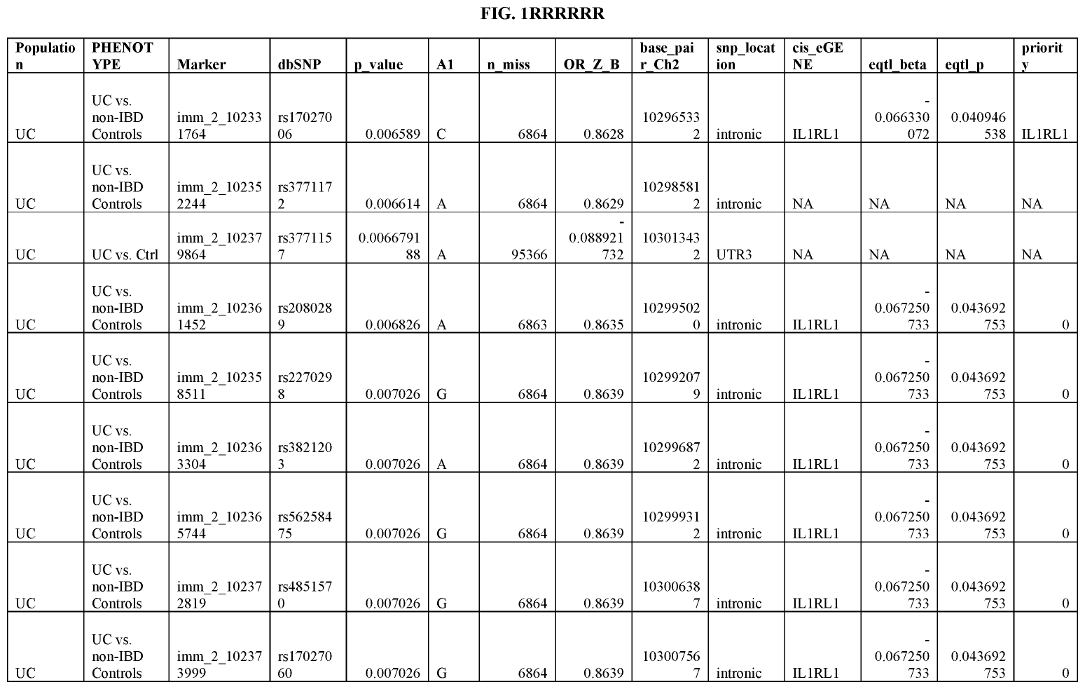

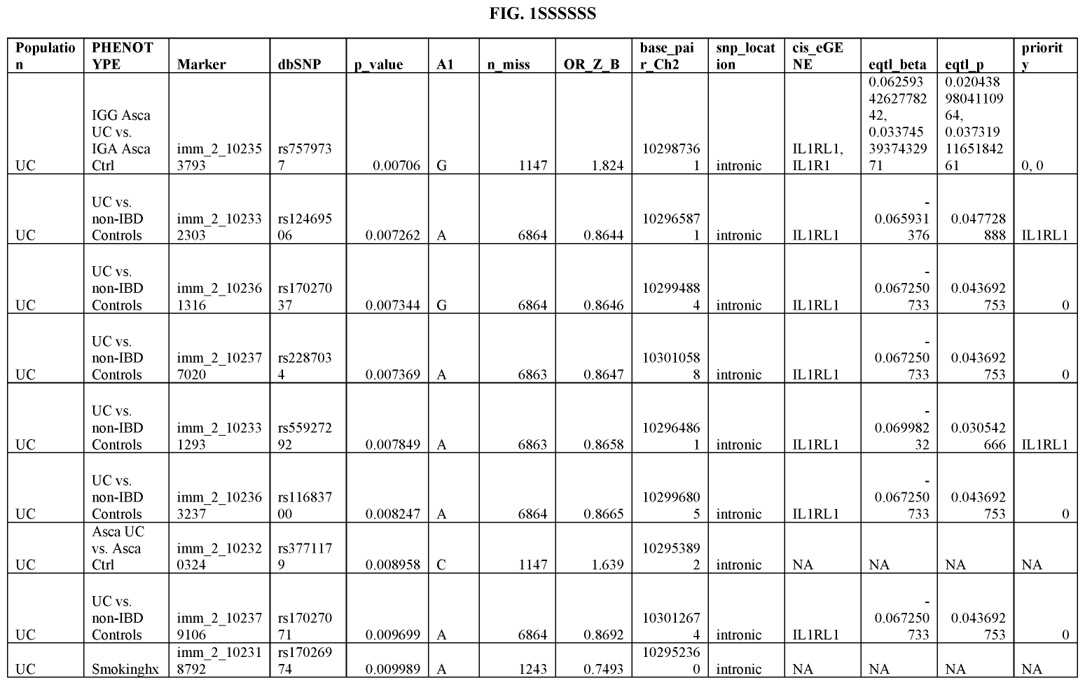

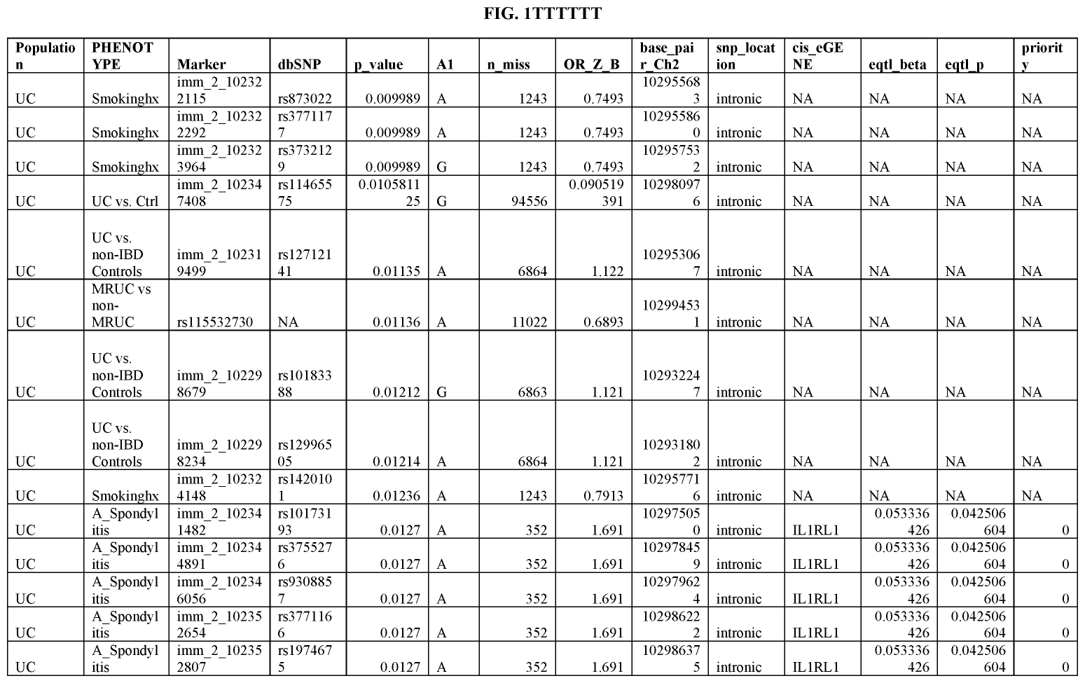

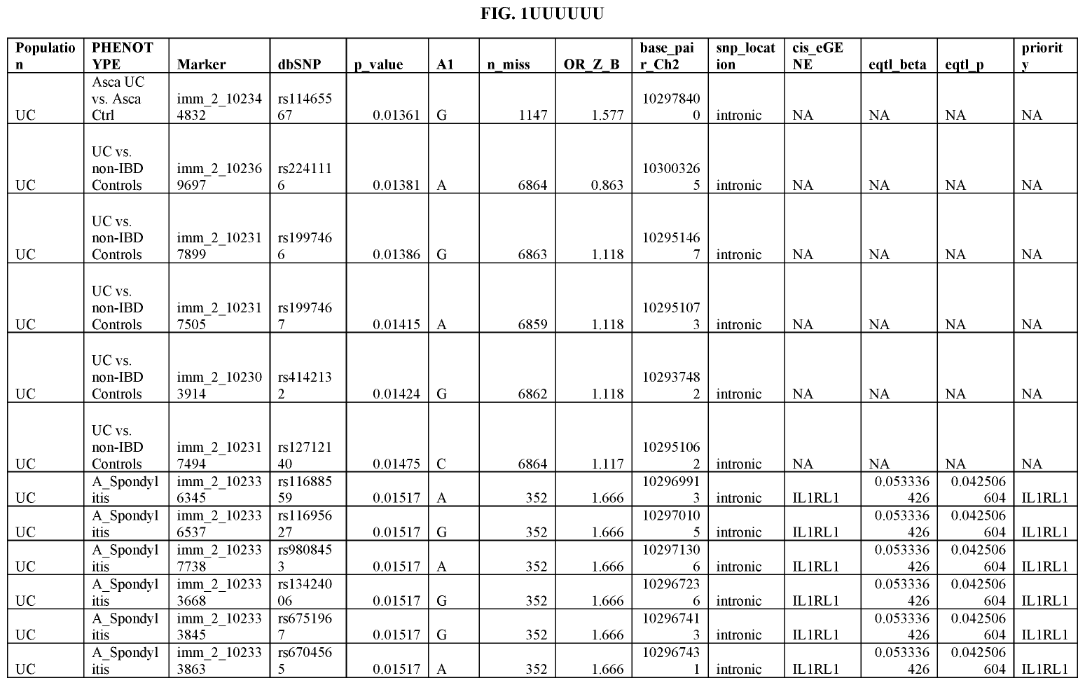

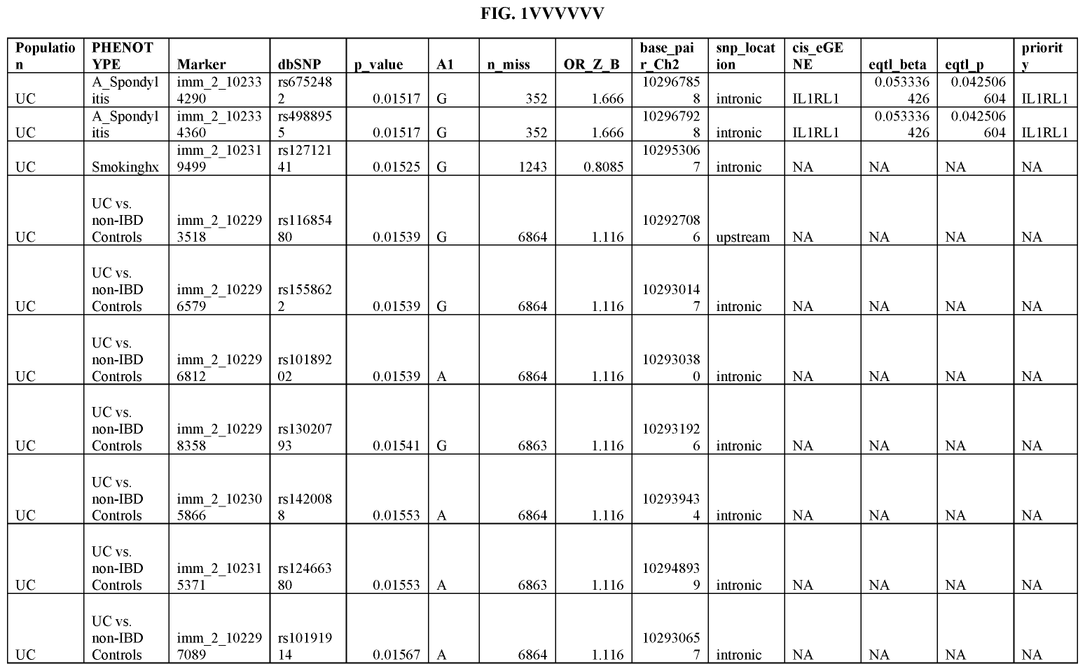

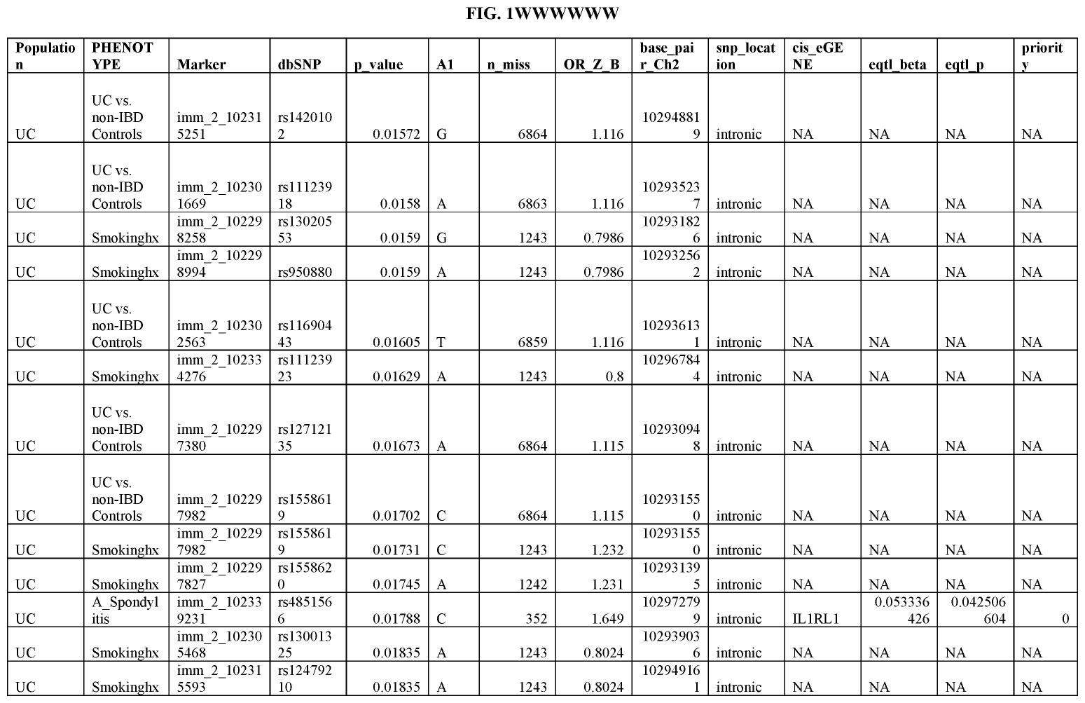

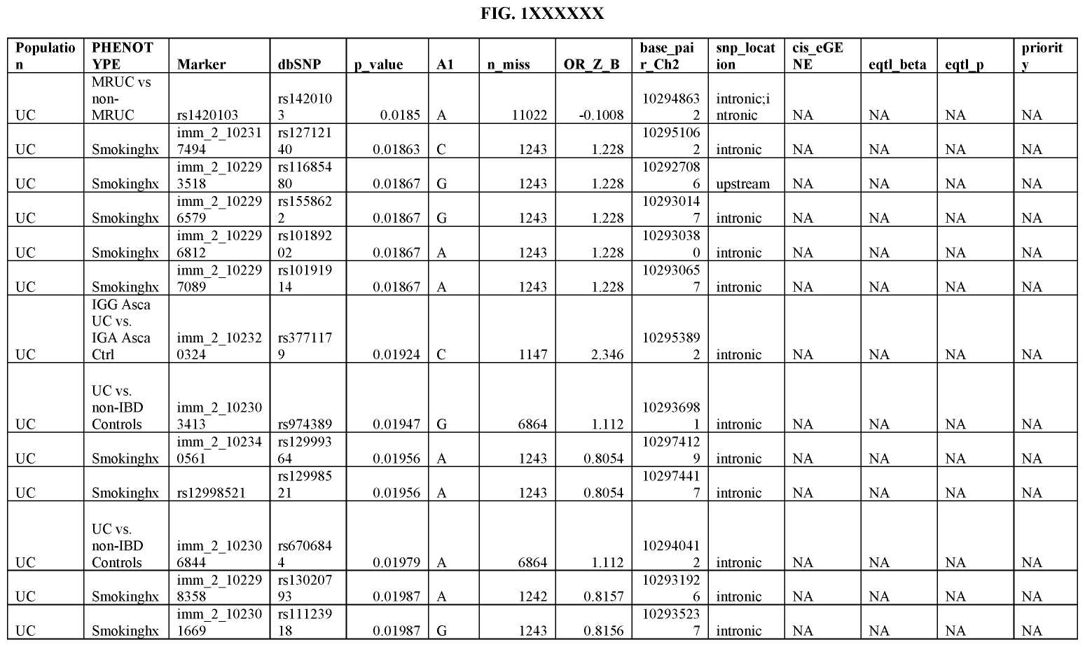

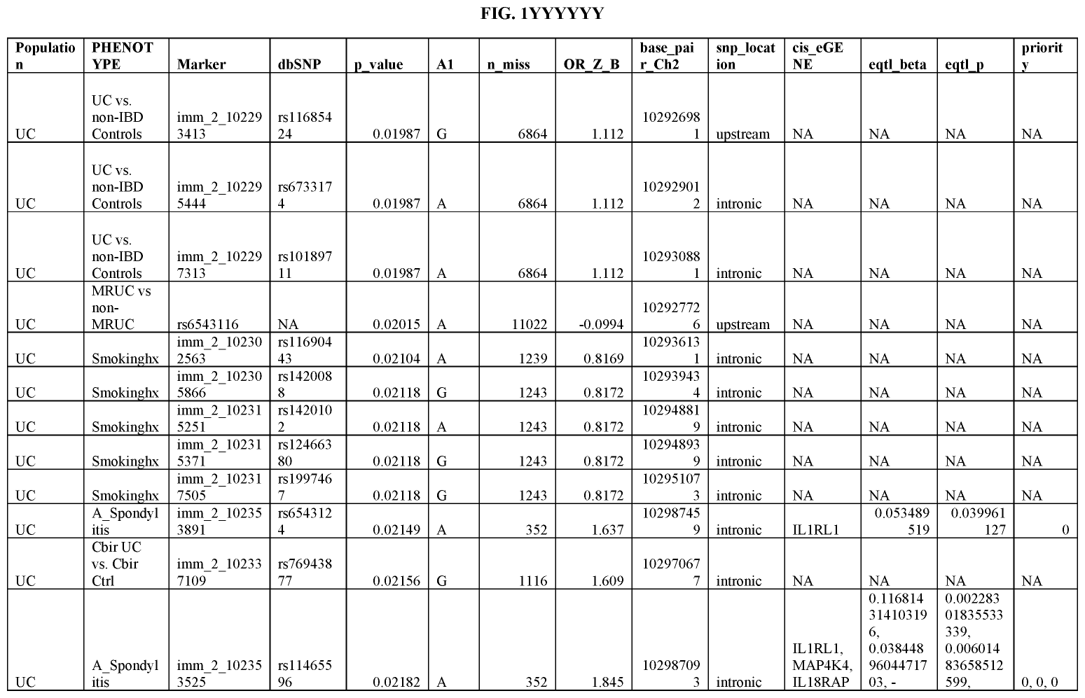

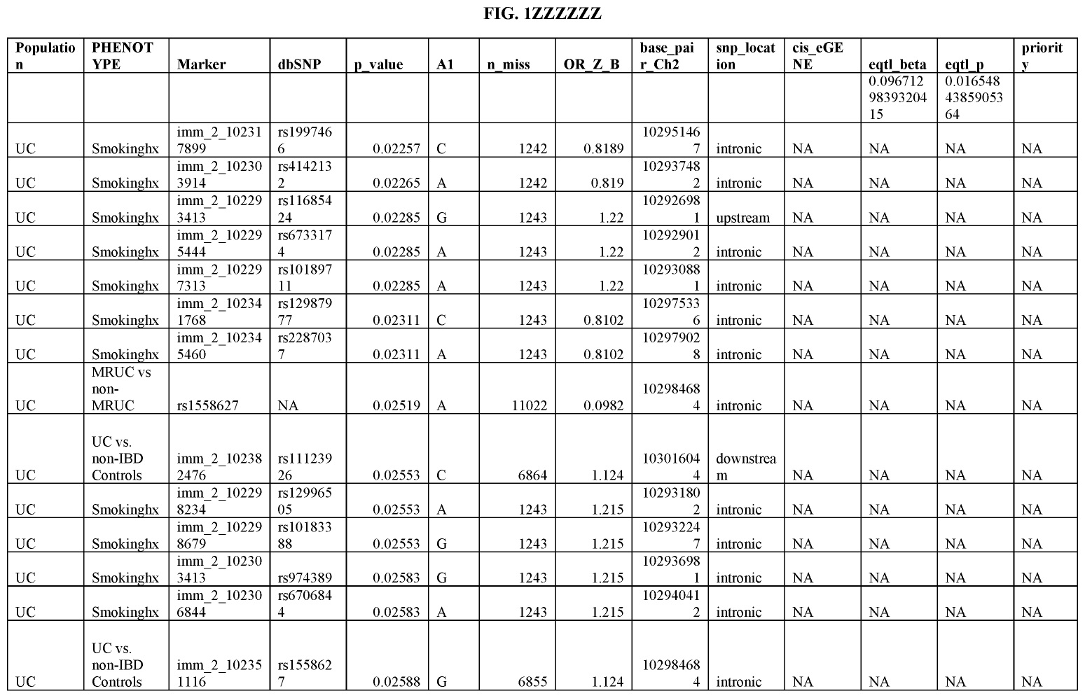

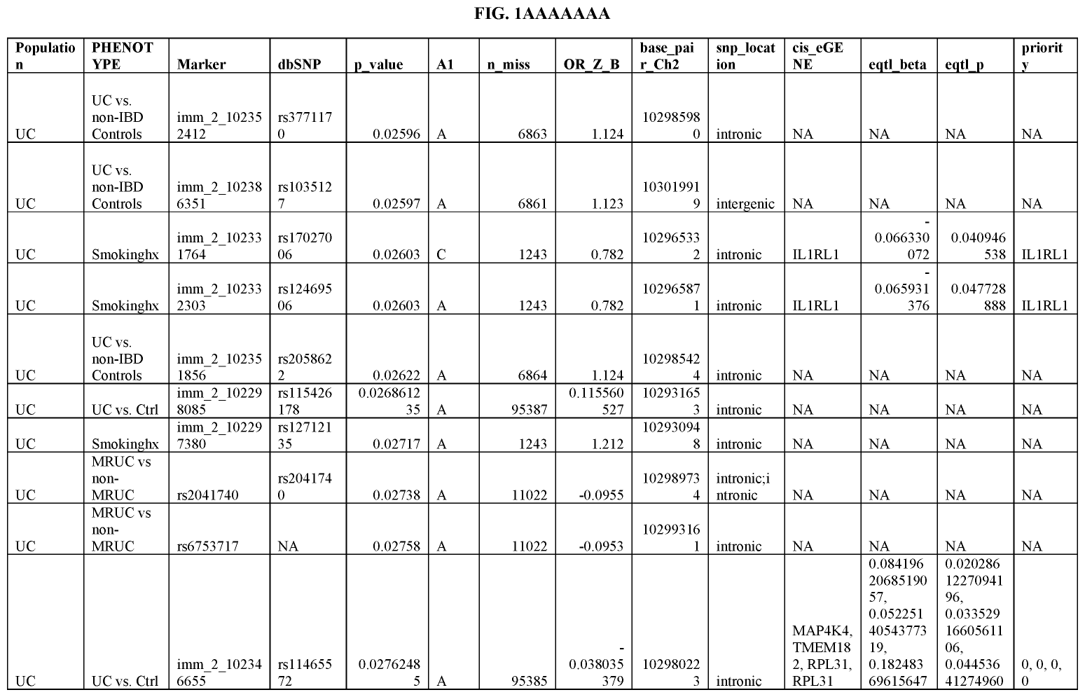

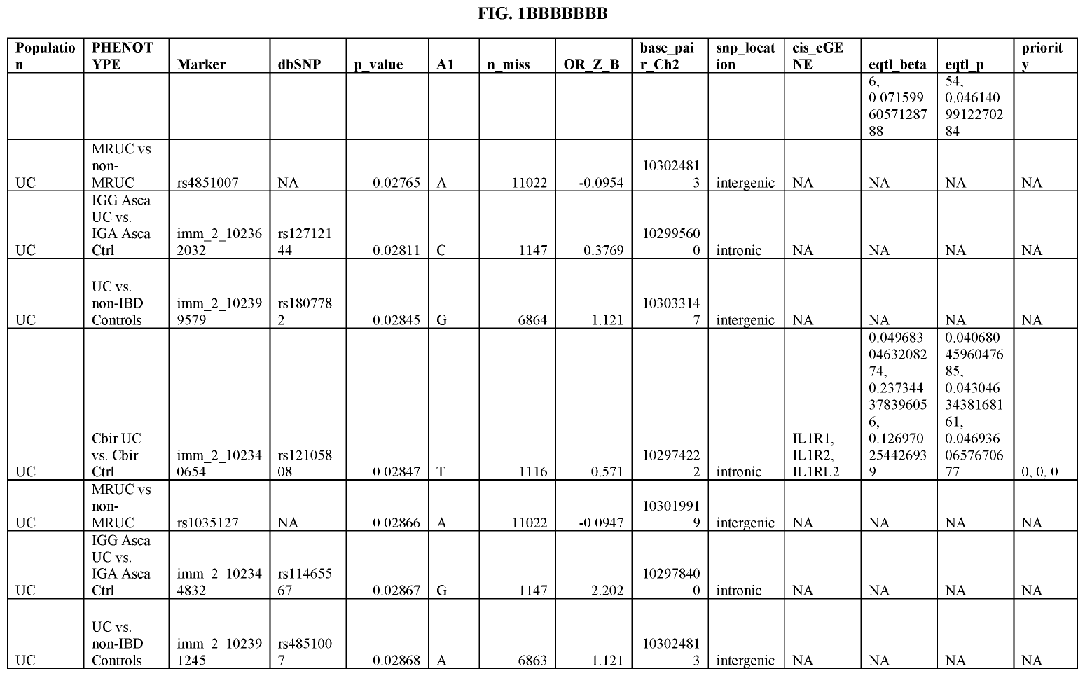

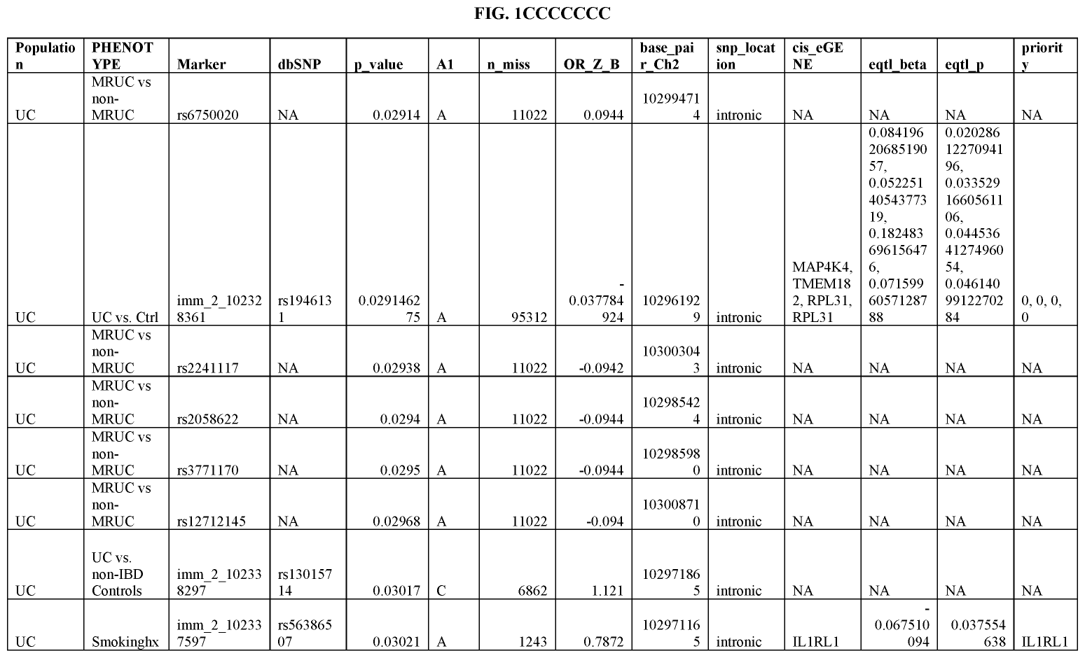

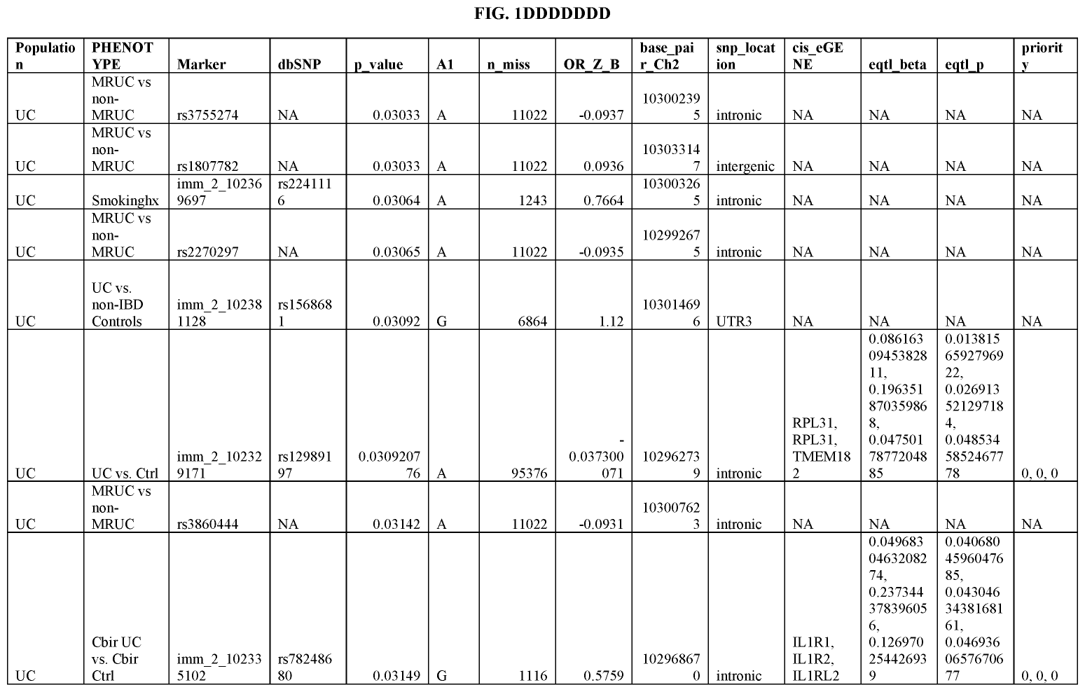

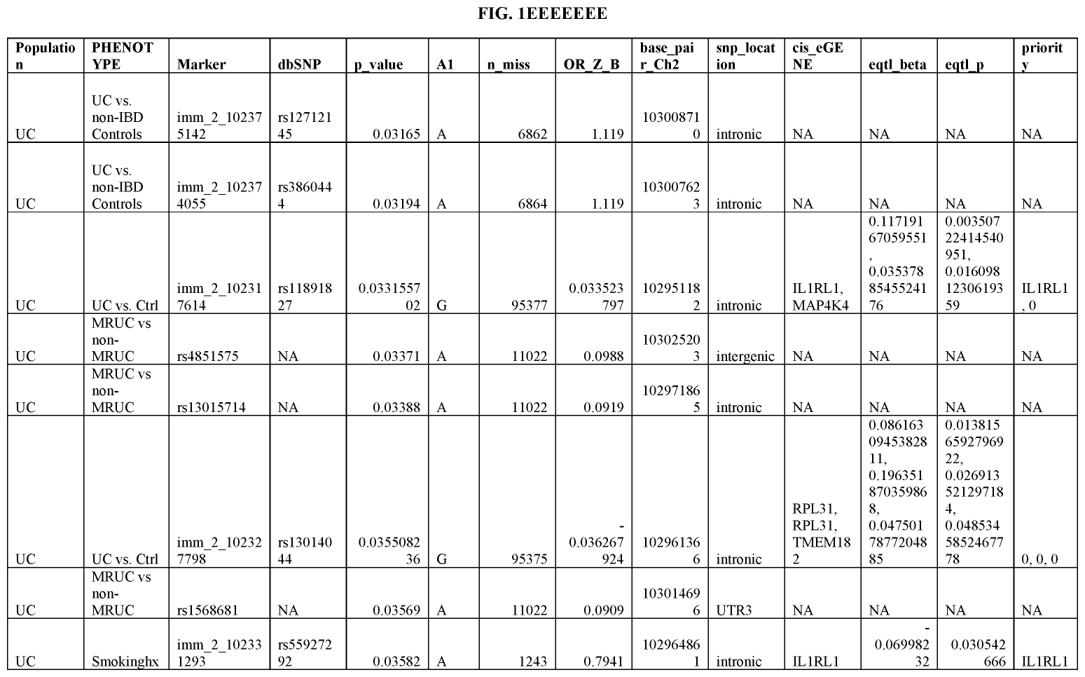

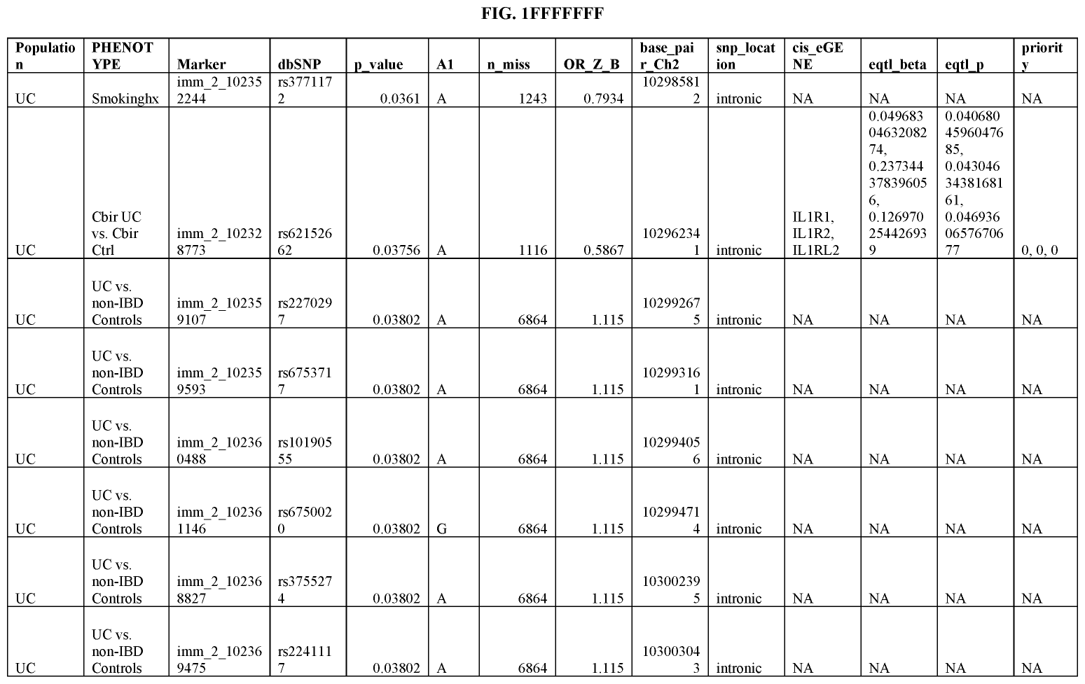

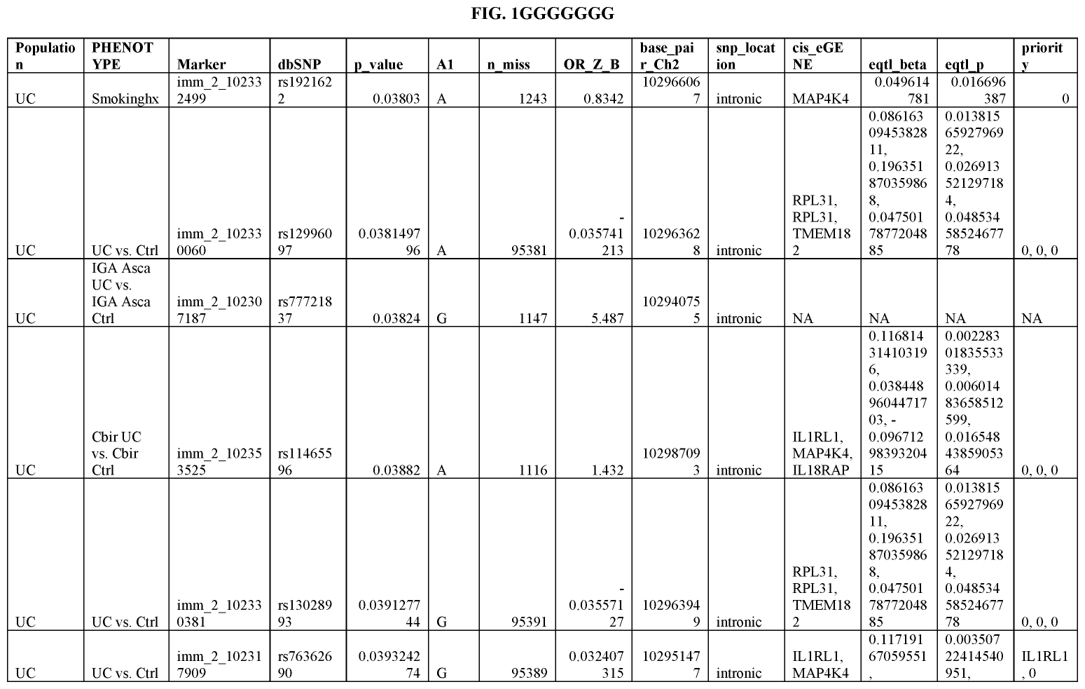

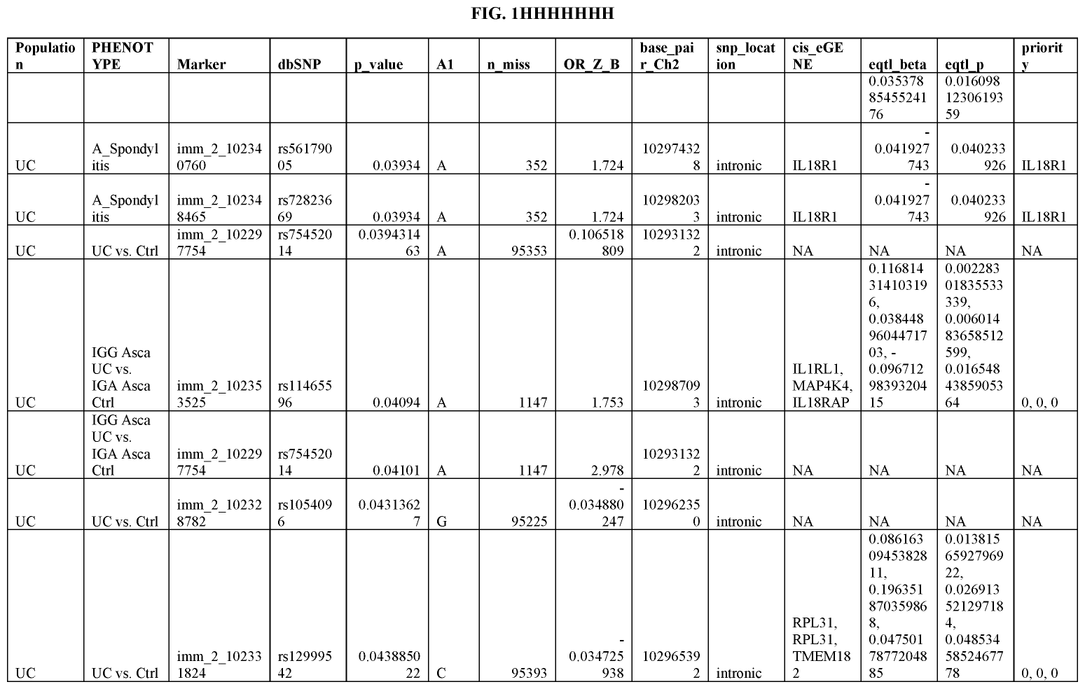

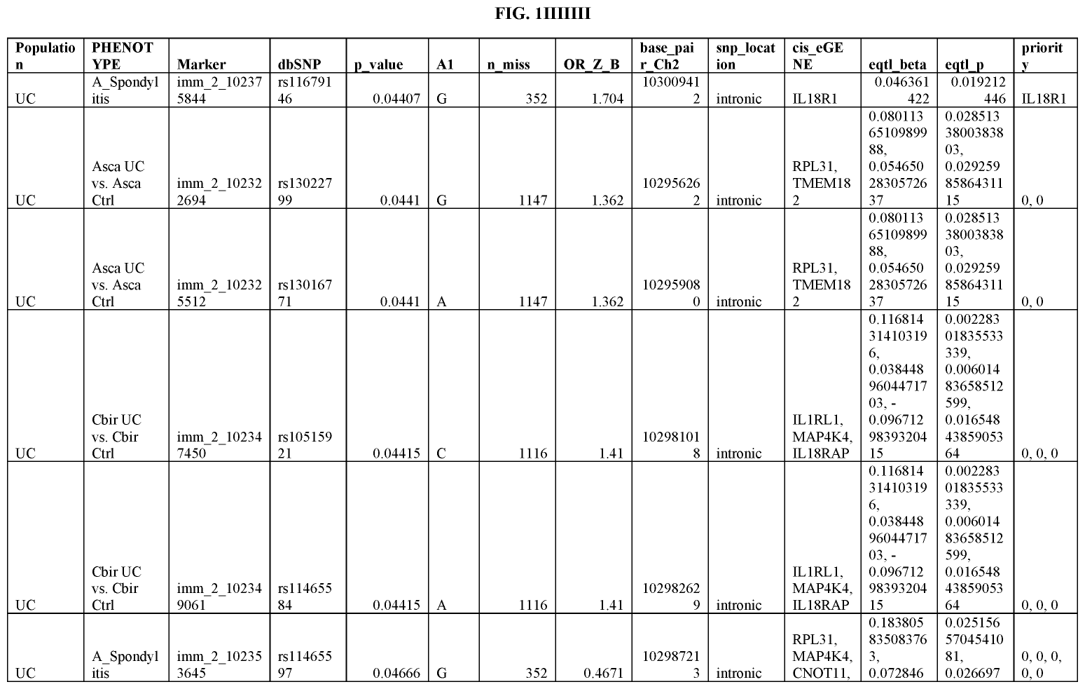

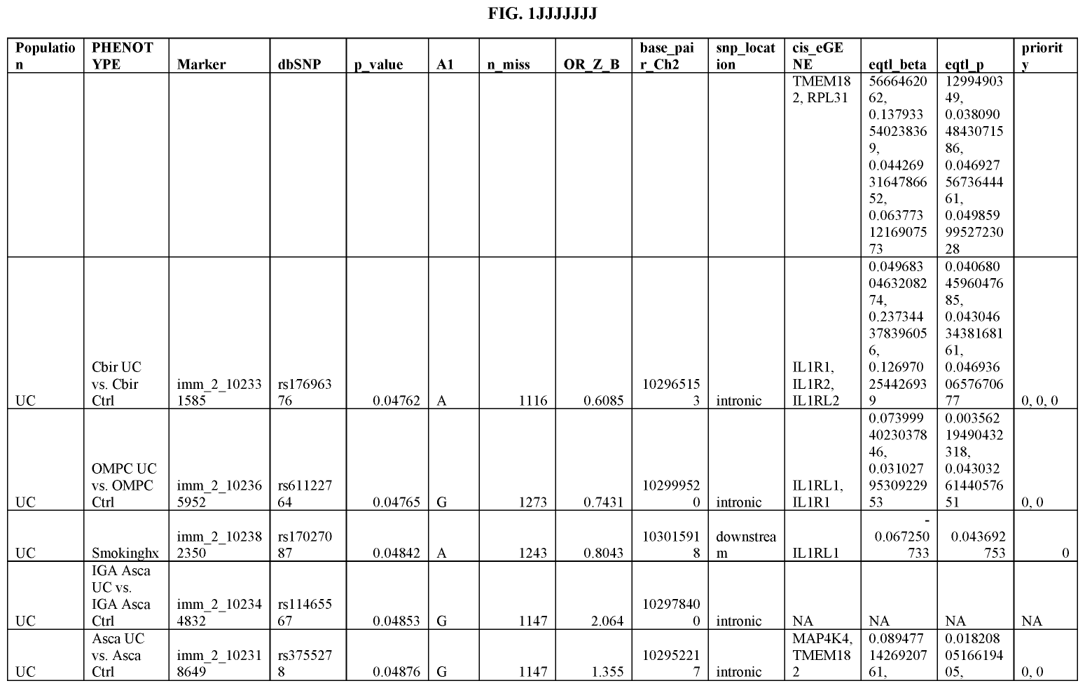

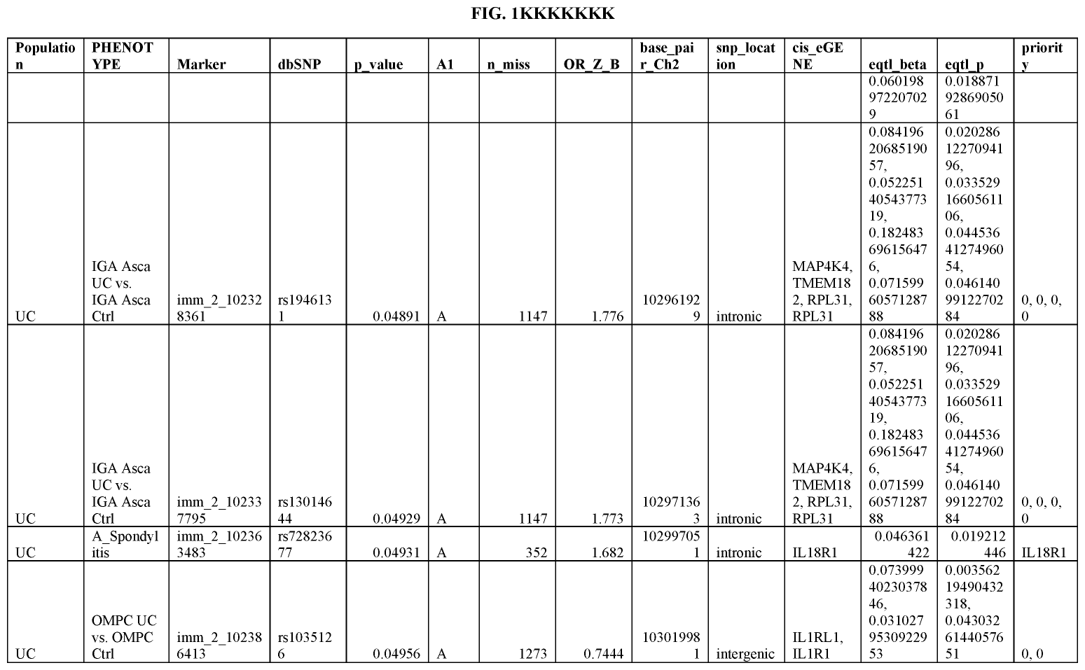

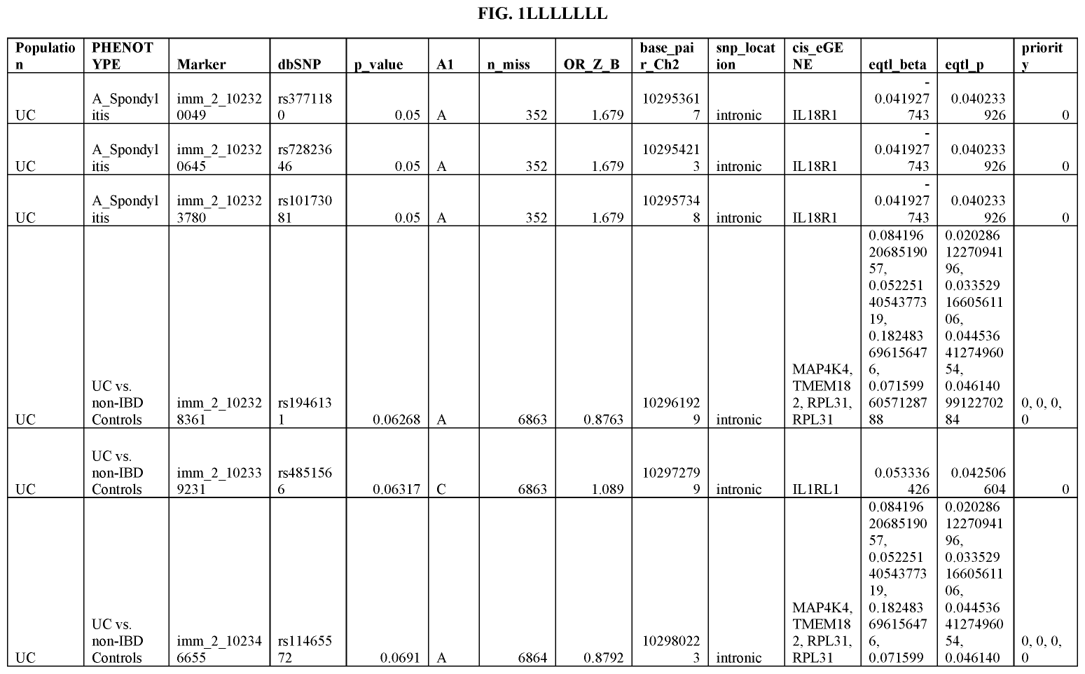

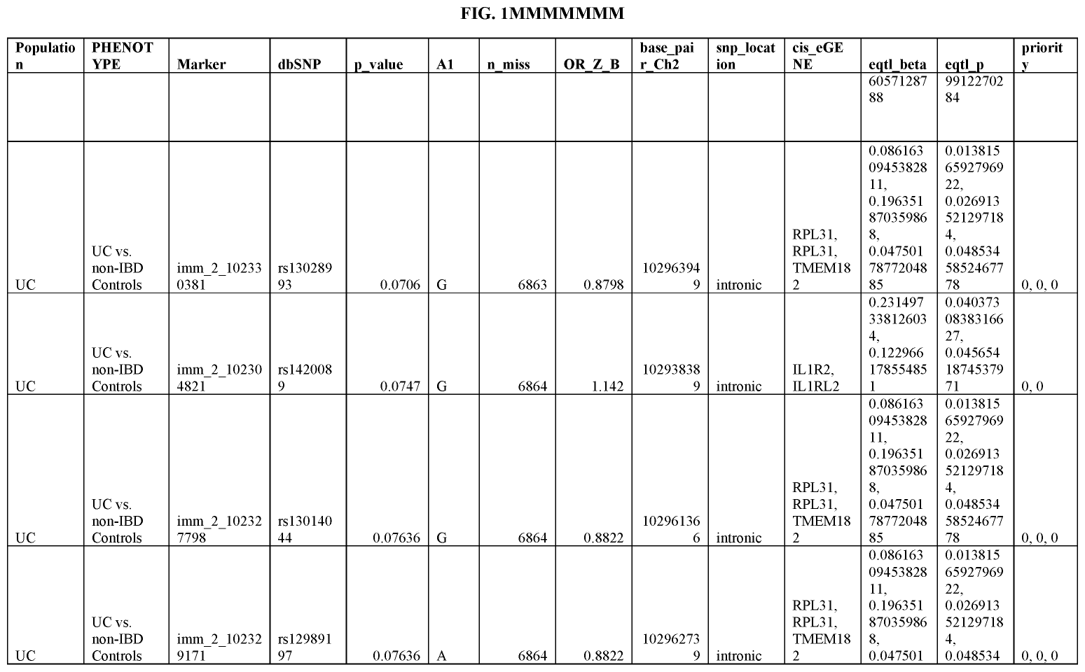

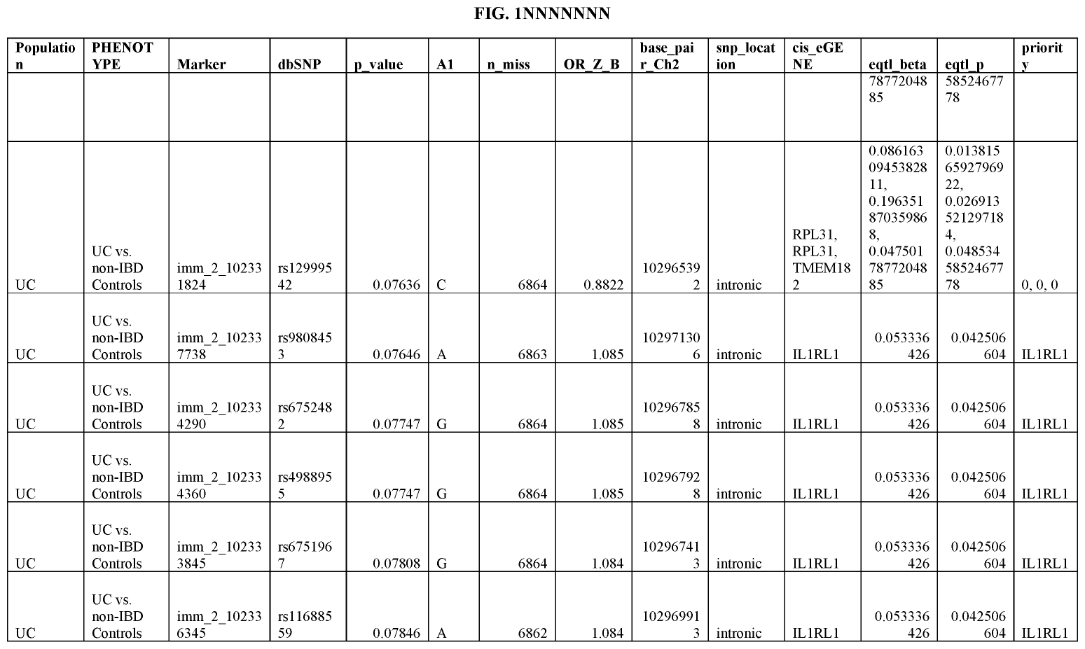

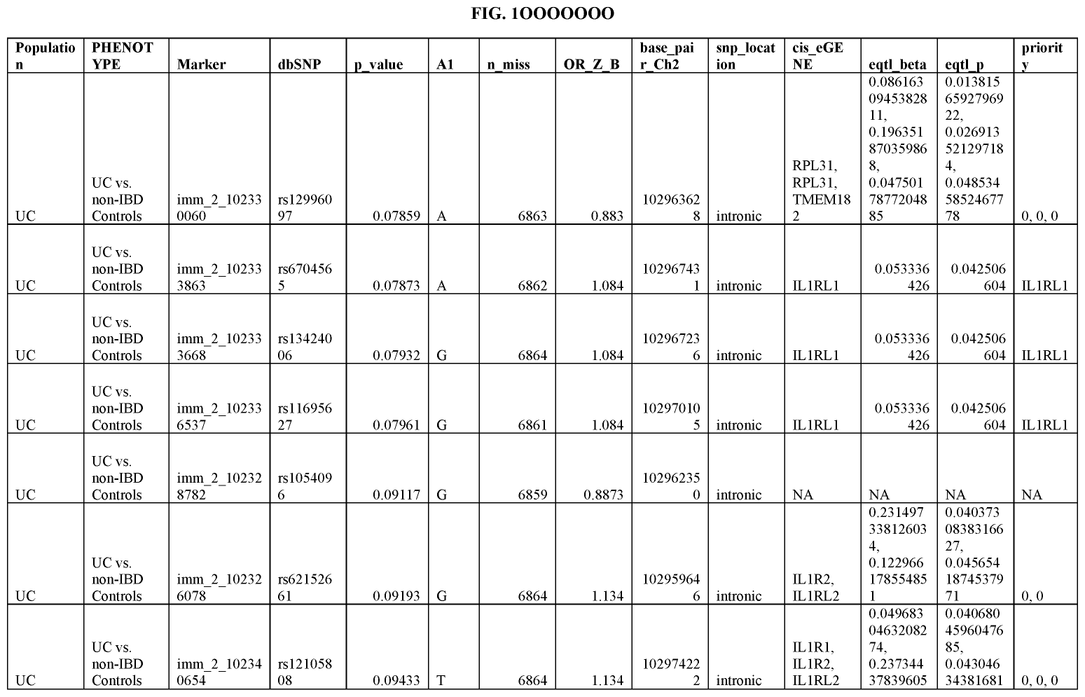

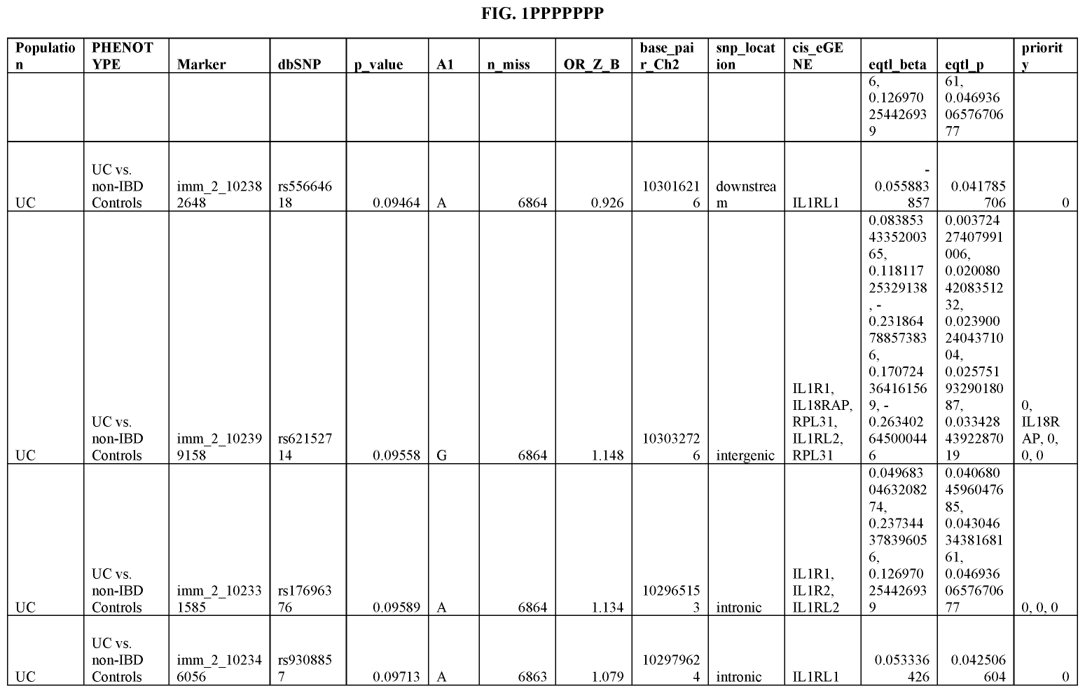

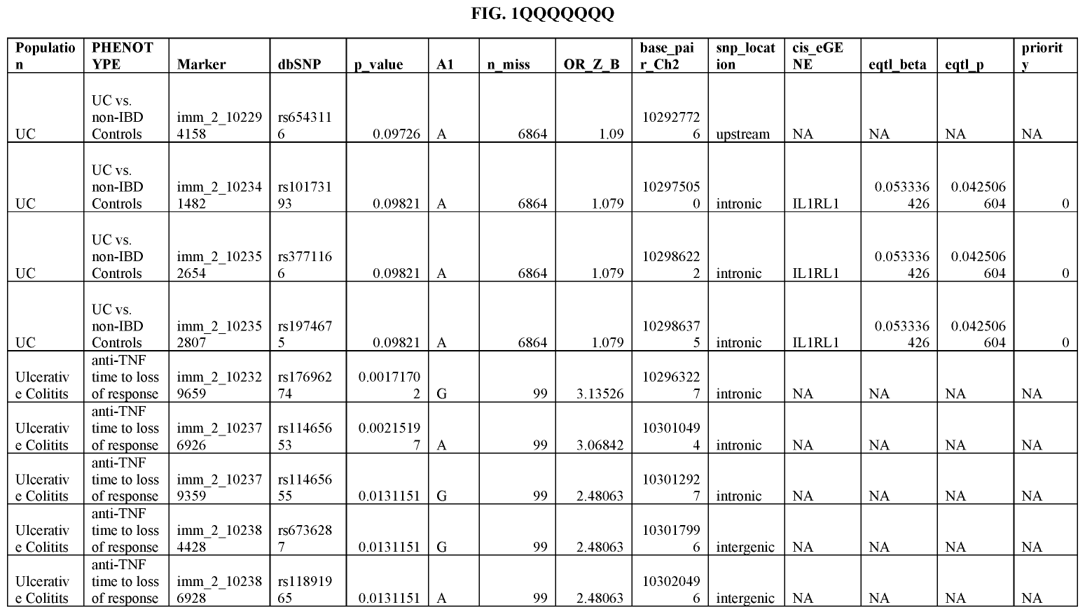

Disclosed herein are genotypes comprising one or more single nucleotide polymorphisms (SNPs or indels (insertion/deletion) at the IL18R1 genetic locus (e.g., IL18R1 risk genotype), according to the following embodiments:
1. A IL18R1 risk genotype comprising one or more SNPs and/or indels at the IL18R1 genetic locus.
2. The IL18R1 risk genotype of embodiment 1, comprising one or more SNPs and/or indels at rs13001325, rs1420101, rs12479210, rs950880, rs13020553, rs13019081, rs12712141, rs2287037, rs1420102, rs12466380, rs1997467, rs1558619, rs1420088, rs12999364, rs4142132, rs12987977, rs11690443, rs1362350, rs12996505, rs873022, rs974389, rs3771177, rs3732129, rs17026974, rs6706844, rs13020793, rs11685480, rs1558622, rs10183388, rs12712135, rs10189711, rs11685424, rs10189202, rs10191914, rs11123918, rs1968171, rs6733174, rs59247511, rs1558620, rs1921622, rs12998521, rs13017455, rs1362349, rs11123923, rs10190555, rs1035127, rs17027087, rs2080289, rs4851570, rs17027060, rs12712145, rs1420098, rs3732123, rs2287034, rs3860444, rs3821203, rs56258475, rs2270298, rs4851006, rs6710885, rs1568681, rs2241117, rs17027037, rs2270297, rs6753717, rs3755274, rs17027071, rs6750020, rs17027006, rs11683700, rs2058622, rs4851007, rs3732126, rs1807782, rs12469506, rs4851575, rs3771172, rs11465633, rs1135354, rs1558627, rs55927292, rs3771171, rs13015714, rs2160202, rs55883125, rs2041740, rs1035130, rs1420103, rs67723747, rs6543116, rs55664618, rs4851005, rs17027056, rs1420089, rs62152661, rs1420095, rs56030066, rs62152714, rs17696376, rs12105808, rs78248680, rs56151044, rs62152662, rs17651485, rs3771170, rs11123926, rs76721133, rs4988955, rs9807962, rs9808453, rs13424006, rs11695627, rs3771166, rs10173193, rs11465575, rs4851566, rs9308857, rs1974675, rs6751967, rs3771162, rs56386507, rs1997466, rs12712140, and rs1362348, or a SNP and/or indel in linkage disequilibrium therewith, or a combination thereof
3. The IL18R1 risk genotype of any previous embodiment, comprising one or more SNPs and/or indels at rs13001325.
4. The IL18R1 risk genotype of any previous embodiment, comprising one or more SNPs and/or indels at rs1420101.
5. The IL18R1 risk genotype of any previous embodiment, comprising one or more SNPs and/or indels at rs12479210.
6. The IL18R1 risk genotype of any previous embodiment, comprising one or more SNPs and/or indels at rs950880.
7. The IL18R1 risk genotype of any previous embodiment, comprising one or more SNPs and/or indels at rs13020553.
8. The IL18R1 risk genotype of any previous embodiment, comprising one or more SNPs and/or indels at rs13019081.
9. The IL18R1 risk genotype of any previous embodiment, comprising one or more SNPs and/or indels at rs12712141.
10. The IL18R1 risk genotype of any previous embodiment, comprising one or more SNPs and/or indels at rs2287037.
11. The IL18R1 risk genotype of any previous embodiment, comprising one or more SNPs and/or indels at rs1420102.
12. The IL18R1 risk genotype of any previous embodiment, comprising one or more SNPs and/or indels at rs12466380.
13. The IL18R1 risk genotype of any previous embodiment, comprising one or more SNPs and/or indels at rs1997467.
14. The IL18R1 risk genotype of any previous embodiment, comprising one or more SNPs and/or indels at rs1558619.
15. The IL18R1 risk genotype of any previous embodiment, comprising one or more SNPs and/or indels at rs1420088.
16. The IL18R1 risk genotype of any previous embodiment, comprising one or more SNPs and/or indels at rs12999364.
17. The IL18R1 risk genotype of any previous embodiment, comprising one or more SNPs and/or indels at rs4142132.
18. The IL18R1 risk genotype of any previous embodiment, comprising one or more SNPs and/or indels at rs12987977.
19. The IL18R1 risk genotype of any previous embodiment, comprising one or more SNPs and/or indels at rs11690443.
20. The IL18R1 risk genotype of any previous embodiment, comprising one or more SNPs and/or indels at rs1362350.
21. The IL18R1 risk genotype of any previous embodiment, comprising one or more SNPs and/or indels at rs12996505.
22. The IL18R1 risk genotype of any previous embodiment, comprising one or more SNPs and/or indels at rs873022.
23. The IL18R1 risk genotype of any previous embodiment, comprising one or more SNPs and/or indels at rs974389.
24. The IL18R1 risk genotype of any previous embodiment, comprising one or more SNPs and/or indels at rs3771177.
25. The IL18R1 risk genotype of any previous embodiment, comprising one or more SNPs and/or indels at rs3732129.
26. The IL18R1 risk genotype of any previous embodiment, comprising one or more SNPs and/or indels at rs17026974.
27. The IL18R1 risk genotype of any previous embodiment, comprising one or more SNPs and/or indels at rs6706844.
28. The IL18R1 risk genotype of any previous embodiment, comprising one or more SNPs and/or indels at rs13020793.
29. The IL18R1 risk genotype of any previous embodiment, comprising one or more SNPs and/or indels at rs11685480.
30. The IL18R1 risk genotype of any previous embodiment, comprising one or more SNPs and/or indels at rs1558622.
31. The IL18R1 risk genotype of any previous embodiment, comprising one or more SNPs and/or indels at rs10183388.
32. The IL18R1 risk genotype of any previous embodiment, comprising one or more SNPs and/or indels at rs12712135.
33. The IL18R1 risk genotype of any previous embodiment, comprising one or more SNPs and/or indels at rs10189711.
34. The IL18R1 risk genotype of any previous embodiment, comprising one or more SNPs and/or indels at rs11685424.
35. The IL18R1 risk genotype of any previous embodiment, comprising one or more SNPs and/or indels at rs10189202.
36. The IL18R1 risk genotype of any previous embodiment, comprising one or more SNPs and/or indels at rs10191914.
37. The IL18R1 risk genotype of any previous embodiment, comprising one or more SNPs and/or indels at rs11123918.
38. The IL18R1 risk genotype of any previous embodiment, comprising one or more SNPs and/or indels at rs1968171.
39. The IL18R1 risk genotype of any previous embodiment, comprising one or more SNPs and/or indels at rs6733174.
40. The IL18R1 risk genotype of any previous embodiment, comprising one or more SNPs and/or indels at rs59247511.
41. The IL18R1 risk genotype of any previous embodiment, comprising one or more SNPs and/or indels at rs1558620.
42. The IL18R1 risk genotype of any previous embodiment, comprising one or more SNPs and/or indels at rs1921622.
43. The IL18R1 risk genotype of any previous embodiment, comprising one or more SNPs and/or indels at rs12998521.
44. The IL18R1 risk genotype of any previous embodiment, comprising one or more SNPs and/or indels at rs13017455.
45. The IL18R1 risk genotype of any previous embodiment, comprising one or more SNPs and/or indels at rs1362349.
46. The IL18R1 risk genotype of any previous embodiment, comprising one or more SNPs and/or indels at rs11123923.
47. The IL18R1 risk genotype of any previous embodiment, comprising one or more SNPs and/or indels at rs10190555.
48. The IL18R1 risk genotype of any previous embodiment, comprising one or more SNPs and/or indels at rs1035127.
49. The IL18R1 risk genotype of any previous embodiment, comprising one or more SNPs and/or indels at rs17027087.
50. The IL18R1 risk genotype of any previous embodiment, comprising one or more SNPs and/or indels at rs2080289.
51. The IL18R1 risk genotype of any previous embodiment, comprising one or more SNPs and/or indels at rs4851570.
52. The IL18R1 risk genotype of any previous embodiment, comprising one or more SNPs and/or indels at rs17027060.
53. The IL18R1 risk genotype of any previous embodiment, comprising one or more SNPs and/or indels at rs12712145.
54. The IL18R1 risk genotype of any previous embodiment, comprising one or more SNPs and/or indels at rs1420098.
55. The IL18R1 risk genotype of any previous embodiment, comprising one or more SNPs and/or indels at rs3732123.
56. The IL18R1 risk genotype of any previous embodiment, comprising one or more SNPs and/or indels at rs2287034.
57. The IL18R1 risk genotype of any previous embodiment, comprising one or more SNPs and/or indels at rs3860444.
58. The IL18R1 risk genotype of any previous embodiment, comprising one or more SNPs and/or indels at rs3821203.
59. The IL18R1 risk genotype of any previous embodiment, comprising one or more SNPs and/or indels at rs56258475.
60. The IL18R1 risk genotype of any previous embodiment, comprising one or more SNPs and/or indels at rs2270298.
61. The IL18R1 risk genotype of any previous embodiment, comprising one or more SNPs and/or indels at rs4851006.
62. The IL18R1 risk genotype of any previous embodiment, comprising one or more SNPs and/or indels at rs6710885.
63. The IL18R1 risk genotype of any previous embodiment, comprising one or more SNPs and/or indels at rs1568681.
64. The IL18R1 risk genotype of any previous embodiment, comprising one or more SNPs and/or indels at rs2241117.
65. The IL18R1 risk genotype of any previous embodiment, comprising one or more SNPs and/or indels at rs17027037.
66. The IL18R1 risk genotype of any previous embodiment, comprising one or more SNPs and/or indels at rs2270297.
67. The IL18R1 risk genotype of any previous embodiment, comprising one or more SNPs and/or indels at rs6753717.
68. The IL18R1 risk genotype of any previous embodiment, comprising one or more SNPs and/or indels at rs3755274.
69. The IL18R1 risk genotype of any previous embodiment, comprising one or more SNPs and/or indels at rs17027071.
70. The IL18R1 risk genotype of any previous embodiment, comprising one or more SNPs and/or indels at rs6750020.
71. The IL18R1 risk genotype of any previous embodiment, comprising one or more SNPs and/or indels at rs17027006.
72. The IL18R1 risk genotype of any previous embodiment, comprising one or more SNPs and/or indels at rs11683700.
73. The IL18R1 risk genotype of any previous embodiment, comprising one or more SNPs and/or indels at rs2058622.
74. The IL18R1 risk genotype of any previous embodiment, comprising one or more SNPs and/or indels at rs4851007.
75. The IL18R1 risk genotype of any previous embodiment, comprising one or more SNPs and/or indels at rs3732126.
76. The IL18R1 risk genotype of any previous embodiment, comprising one or more SNPs and/or indels at rs1807782.
77. The IL18R1 risk genotype of any previous embodiment, comprising one or more SNPs and/or indels at rs12469506.
78. The IL18R1 risk genotype of any previous embodiment, comprising one or more SNPs and/or indels at rs4851575.
79. The IL18R1 risk genotype of any previous embodiment, comprising one or more SNPs and/or indels at rs3771172.
80. The IL18R1 risk genotype of any previous embodiment, comprising one or more SNPs and/or indels at rs11465633.
81. The IL18R1 risk genotype of any previous embodiment, comprising one or more SNPs and/or indels at rs1135354.
82. The IL18R1 risk genotype of any previous embodiment, comprising one or more SNPs and/or indels at rs1558627.
83. The IL18R1 risk genotype of any previous embodiment, comprising one or more SNPs and/or indels at rs55927292.
84. The IL18R1 risk genotype of any previous embodiment, comprising one or more SNPs and/or indels at rs3771171.
85. The IL18R1 risk genotype of any previous embodiment, comprising one or more SNPs and/or indels at rs13015714.
86. The IL18R1 risk genotype of any previous embodiment, comprising one or more SNPs and/or indels at rs2160202.
87. The IL18R1 risk genotype of any previous embodiment, comprising one or more SNPs and/or indels at rs55883125.
88. The IL18R1 risk genotype of any previous embodiment, comprising one or more SNPs and/or indels at rs2041740.
89. The IL18R1 risk genotype of any previous embodiment, comprising one or more SNPs and/or indels at rs1035130.
90. The IL18R1 risk genotype of any previous embodiment, comprising one or more SNPs and/or indels at rs1420103.
91. The IL18R1 risk genotype of any previous embodiment, comprising one or more SNPs and/or indels at rs67723747.
92. The IL18R1 risk genotype of any previous embodiment, comprising one or more SNPs and/or indels at rs6543116.
93. The IL18R1 risk genotype of any previous embodiment, comprising one or more SNPs and/or indels at rs55664618.
94. The IL18R1 risk genotype of any previous embodiment, comprising one or more SNPs and/or indels at rs4851005.
95. The IL18R1 risk genotype of any previous embodiment, comprising one or more SNPs and/or indels at rs17027056.
96. The IL18R1 risk genotype of any previous embodiment, comprising one or more SNPs and/or indels at rs1420089.
97. The IL18R1 risk genotype of any previous embodiment, comprising one or more SNPs and/or indels at rs62152661.
98. The IL18R1 risk genotype of any previous embodiment, comprising one or more SNPs and/or indels at rs1420095.
99. The IL18R1 risk genotype of any previous embodiment, comprising one or more SNPs and/or indels at rs56030066.
100. The IL18R1 risk genotype of any previous embodiment, comprising one or more SNPs and/or indels at rs62152714.
101. The IL18R1 risk genotype of any previous embodiment, comprising one or more SNPs and/or indels at rs17696376.
102. The IL18R1 risk genotype of any previous embodiment, comprising one or more SNPs and/or indels at rs12105808.
103. The IL18R1 risk genotype of any previous embodiment, comprising one or more SNPs and/or indels at rs78248680.
104. The IL18R1 risk genotype of any previous embodiment, comprising one or more SNPs and/or indels at rs56151044.
105. The IL18R1 risk genotype of any previous embodiment, comprising one or more SNPs and/or indels at rs62152662.
106. The IL18R1 risk genotype of any previous embodiment, comprising one or more SNPs and/or indels at rs17651485.
107. The IL18R1 risk genotype of any previous embodiment, comprising one or more SNPs and/or indels at rs3771170.
108. The IL18R1 risk genotype of any previous embodiment, comprising one or more SNPs and/or indels at rs11123926.
109. The IL18R1 risk genotype of any previous embodiment, comprising one or more SNPs and/or indels at rs76721133.
110. The IL18R1 risk genotype of any previous embodiment, comprising one or more SNPs and/or indels at rs4988955.
111. The IL18R1 risk genotype of any previous embodiment, comprising one or more SNPs and/or indels at rs9807962.
112. The IL18R1 risk genotype of any previous embodiment, comprising one or more SNPs and/or indels at rs9808453.
113. The IL18R1 risk genotype of any previous embodiment, comprising one or more SNPs and/or indels at rs13424006.
114. The IL18R1 risk genotype of any previous embodiment, comprising one or more SNPs and/or indels at rs11695627.
115. The IL18R1 risk genotype of any previous embodiment, comprising one or more SNPs and/or indels at rs3771166.
116. The IL18R1 risk genotype of any previous embodiment, comprising one or more SNPs and/or indels at rs10173193.
117. The IL18R1 risk genotype of any previous embodiment, comprising one or more SNPs and/or indels at rs11465575.
118. The IL18R1 risk genotype of any previous embodiment, comprising one or more SNPs and/or indels at rs4851566.
119. The IL18R1 risk genotype of any previous embodiment, comprising one or more SNPs and/or indels at rs9308857.
120. The IL18R1 risk genotype of any previous embodiment, comprising one or more SNPs and/or indels at rs1974675.
121. The IL18R1 risk genotype of any previous embodiment, comprising one or more SNPs and/or indels at rs6751967.
122. The IL18R1 risk genotype of any previous embodiment, comprising one or more SNPs and/or indels at rs3771162.
123. The IL18R1 risk genotype of any previous embodiment, comprising one or more SNPs and/or indels at rs56386507.
124. The IL18R1 risk genotype of any previous embodiment, comprising one or more SNPs and/or indels at rs1997466.
125. The IL18R1 risk genotype of any previous embodiment, comprising one or more SNPs and/or indels at rs12712140.
126. The IL18R1 risk genotype of any previous embodiment, comprising one or more SNPs and/or indels at rs1362348.
127. The IL18R1 risk genotype of any previous embodiment, comprising one or more SNPs and/or indels at a SNP listed in Table 1.
128. The IL18R1 risk genotype of embodiment 127, wherein the one or more SNP and/or indels is associated with stricturing.
129. The IL18R1 risk genotype of embodiment 128, wherein the stricturing is isolated to an ileocolonic region of an intestine.
130. The IL18R1 risk genotype of any previous embodiment, comprising one or more SNPs and/or indels at a SNP listed in Table 2.
131. The IL18R1 risk genotype of embodiment 130, wherein the one or more SNPs and/or indels is associated with a risk of a subject developing morphological defects in ileal Paneth cells.
132. The IL18R1 risk genotype of any previous embodiment, comprising one or more SNPs and/or indels at a SNP listed in Table 3.
133. The IL18R1 risk genotype of any previous embodiment, comprising one or more SNPs and/or indels at a SNP listed in Table 4.
134. The IL18R1 risk genotype of any previous embodiment, comprising one or more SNPs and/or indels at a SNP listed in Table 5.
135. The IL18R1 risk genotype of any previous embodiments, comprising one or more SNPs and/or indels at a SNP listed in FIG. 1A to FIG. 1QQQQQQQ.
136. The IL18R1 risk genotype of any previous embodiments, comprising one or more SNPs or indels at rs1921622, rs2287037, rs1974675, rs2041739, rs76362690, rs2287037, or rs80256362, or a SNP or indel in linkage disequilibrium (LD) therewith.
137. The IL18R1 risk genotype of any previous embodiment, wherein the SNP at rs1921622 comprises an “A” or a “G” on a forward DNA strand encoding the SNP.
138. The IL18R1 risk genotype of any previous embodiments, wherein the SNP at rs2287037 comprises an “A” or a “G” on a reverse DNA strand encoding the SNP.
139. The IL18R1 risk genotype of any previous embodiments, wherein the SNP at rs1974675 comprises a “C” or a “T” on a reverse DNA strand encoding the SNP.
140. The IL18R1 risk genotype of any previous embodiments, wherein the SNP at rs2041739 comprises an “A” or a “G” on a reverse DNA strand encoding the SNP.
141. The IL18R1 risk genotype of any previous embodiments, wherein the SNP at rs76362690 comprises an “A” or a “G” on a forward DNA strand encoding the SNP.
142. The IL18R1 risk genotype of any previous embodiments, wherein the SNP at rs2287037 comprises an “A” or a “G” on a reverse DNA strand encoding the SNP.
143. The IL18R1 risk genotype of any previous embodiments, wherein the SNP at rs80256362 comprises an “A” or a “G” on a forward DNA strand encoding the SNP.
144. The IL18R1 risk genotype of any previous embodiments, comprising 1, 2, 3, 4, 5, 6, 7, 8, 9, 10, 11, 12, 13, 14, or 15 SNPs and/or indels.
145. The IL18R1 risk genotype of any previous embodiments, wherein the genotype is associated with a risk that a subject has, or will develop, inflammatory bowel disease (IBD), Crohn's disease (CD), or ulcerative colitis (UC), as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰.
146. The IL18R1 risk genotype of any previous embodiments, wherein the genotype is associated with a risk that the subject has, or will develop, a subclinical phenotype of the disease or condition as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰.
147. The IL18R1 risk genotype of any previous embodiments, wherein the genotype comprises one or more SNPs and/or indels in linkage disequilibrium with rs1921622 as determined by an r² value of at least about 0.80, about 0.85, about 0.90, about 0.95, or about 1.0
148. The IL18R1 risk genotype of any previous embodiments, wherein the genotype comprises SEQ ID NO: 1.
149. The IL18R1 risk genotype of any previous embodiments, wherein the genotype comprises SEQ ID NO: 2.
150. The IL18R1 risk genotype of any previous embodiments, wherein the genotype comprises SEQ ID NO: 3.
151. The IL18R1 risk genotype of any previous embodiments, wherein the genotype comprises SEQ ID NO: 4.
152. The IL18R1 risk genotype of any previous embodiments, wherein the genotype comprises SEQ ID NO: 5.
153. The IL18R1 risk genotype of any previous embodiments, wherein the genotype comprises SEQ ID NO: 6.
154. The IL18R1 risk genotype of any previous embodiments, wherein the genotype comprises SEQ ID NO: 7.
155. The IL18R1 risk genotype of embodiment 1498, wherein the “N” within SEQ ID NO: 1 comprises an “A” or a “G.”
156. The IL18R1 risk genotype of embodiment 149, wherein the “N” within SEQ ID NO: 2 comprises an “A” or a “G.”
157. The IL18R1 risk genotype of embodiment 150, wherein the “N” within SEQ ID NO: 3 comprises a “C” or a “T.”
158. The IL18R1 risk genotype of embodiment 151, wherein the “N” within SEQ ID NO: 4 comprises an “A” or a “G.”
159. The IL18R1 risk genotype of embodiment 152, wherein the “N” within SEQ ID NO: 5 comprises an “A” or a “G.”
160. The IL18R1 risk genotype of embodiment 153, wherein the “N” within SEQ ID NO: 6 comprises an “A” or a “G.”
161. The IL18R1 risk genotype of embodiment 154, wherein the “N” within SEQ ID NO: 7 comprises an “A” or a “G.”

Clinical and Subclinical Phenotype Associations

In some instances, a presence or an absence of a SNP and/or indel in a subject is associated with a particular phenotype (e.g., disease or condition), or subclinical phenotype, such as those described herein. In some embodiments, the method comprises determining whether the subject has an allele associated with the disease or condition (“risk allele”). Non-limiting examples of clinical phenotypes include inflammatory bowel disease (IBD), Crohn's disease (CD), ulcerative colitis (UC), multiple sclerosis (MS), primary sclerosing cholangitis (PSC), Pancolitis (e.g., UC which affects the entire large intestine), Proctitis (e.g., inflammation of the rectum), Iritis (e.g., inflammation of the iris), Thrombosis (e.g., formation of blood clot inside a blood vessel), Uveitis (e.g., inflammation of the eye, of the uvea), Spondylitis (e.g., inflammation of the spine), arthralgias (e.g., inflammation of the joints), nodosum, perianal Crohn's disease (pCD), Psoriasis (e.g., inflammation of the skin), asthma, Celiacs disease, primary billary cihrosis, and oral ulcers. In some instances, a SNP disclosed herein is associated with IBD. In some instances, a SNP disclosed herein is associated with CD. In some instances, a SNP disclosed herein is associated with UC. In some instances, the SNP disclosed herein is associated with MS. In some instances, the SNP disclosed herein is associated with PSC. In some instances, the SNP disclosed herein is associated with Pancolitis. In some instances, the SNP disclosed herein is associated with Proctitis. In some instances, the SNP disclosed herein is associated with Iritis. In some instances, the SNP disclosed herein is associated with Thrombosis. In some instances, the SNP disclosed herein is associated with Uveitis. In some instances, the SNP disclosed herein is associated with Spondylitis. In some instances, the SNP disclosed herein is associated with arthralgias. In some instances, the althralgia comprises rheumatoid arthritis (RA). In some instances, the SNP disclosed herein is associated with nodosum. In some instances, the SNP disclosed herein is associated with pCD. In some instances, the SNP disclosed herein is associated with Psoriasis. In some instances, the SNP disclosed herein is associated with oral ulcers. In some instances, a SNP is associated with clinical phenotype (e.g., one or the diseases disclosed herein) in a particular location of the intestine. In some instances, the location comprises the ileal, ileocolonic, or colonic region of the intestine, or a combination thereof.

Disclosed herein are SNPs associated with a subclinical phenotype of a disease or disorder disclosed herein. A subclinical phenotype may be a specific diagnosable disease or condition, or metric to measure disease progression that is characteristic of severe or unusual forms of disease. Non-limiting examples of IBD subclinical phenotypes include, but are not limited to, non-stricturing disease, stricturing disease, stricturing and penetrating disease, a time to first surgery, a time to a second surgery, Paneth cell morphologies, and non-response or loss of response to one or more standard therapies. Non-limiting examples of standard therapy of inflammatory disease include glucocorticosteriods, anti-TNF therapy, anti-a4-b7 therapy (vedolizumab), anti-IL12p40 therapy (ustekinumab), Thalidomide, and Cytoxin. In some instances, a SNP is associated with non-response or loss of response to anti-TNF therapy. In some embodiments, a presence of one or more SNPs in a sample obtained from a subject is indicative that the subject has, or will develop, non-response or loss of response to an anti-TNF therapy. Paneth cell morphological phenotypes were determined using the classification set forth in VanDussen et al., “Genetic Variants Synthesize the Produce Paneth Cell Phenotypes That Define Subtypes of Crohn's Disease,” Gastroenterology 2014; 146:200-209.

Disclosed herein, in some embodiments, are SNPs associated with a time to a first surgery and/or time to a second surgery. Time to a first surgery, and time to second surgery, are subclinical phenotypes used to identify subjects at risk for severe forms of disease. In the context of inflammatory bowel disease, a time to first surgery may be a time from a symptom of the inflammatory bowel disease to a surgery. The time to first surgery may be a time from first diagnosis of the IBD to a time of a first surgery. The time to second surgery may be a time from a first surgery to the time of a second surgery. The first and/or second surgery may comprise surgery on at least a portion of the gastrointestinal tract of the subject. Non-limiting surgeries include an intestinal resection, colectomy, perianal surgery, and stricturoplasty. The symptom may be a symptom described herein. The portion of the gastrointestinal tract may be selected from the anus, the colon, the large intestine, the small intestine, the stomach, and the esophagus.

Disclosed herein, in some embodiments, are SNPs that are associated with a faster progression to surgery, as compared to an individual who does not carry the SNP. A faster progression to surgery is indicative of complicated disease, often resistant to therapy. In some embodiments, a presence of one or more SNPs in a sample obtained from a subject is indicative that the subject has, or will develop, complicated disease behavior characterized by a faster progression to a first and/or second surgery. A “first surgery,” as disclosed herein, refers to the first surgical treatment (e.g., colectomy or resection) of a disease or disorder described herein in a subject. A “second surgery,” as used here, refers to the second surgical treatment of the same disease or disorder in the subject. In some instances, a SNP disclosed herein is associated with a first time from a first symptom of the inflammatory bowel disease to a first surgery. In some instances, a SNP disclosed herein is associated with a first time from a diagnosis of the inflammatory bowel disease to a first surgery. In some instances, a SNP disclosed herein is associated with a time from an age to a first surgery. The first time may be about one year to about fifteen years. The first time may be about two years to about twelve years. The first time may be about four years to about ten years. The first time may be about four years to about eight years. In some instances, the time to a first colectomy for a subject with mrUC comprises less than 60 months.

In some instances, a SNP disclosed herein is associated with a second time from a first surgery to a second surgery. The second time may be about one year to about fifteen years. The second time may be about two years to about twelve years. The second time may be about four years to about ten years. The second time may be about four years to about eight years. The time to first surgery for patients carrying a risk allele may be about three years to about nine years. The time to first surgery for patients carrying a risk allele may be about four years to about eight years. The time to first surgery for patients for a risk allele may be about three years to about seven years. The time to first surgery for patients for a risk allele may be about seven years. The time to first surgery for patients homozygous for a non-risk minor allele may be about ten years. The time to first surgery for patients homozygous for a non-risk minor allele may be greater than about ten years. The time to first surgery for patients homozygous for a non-risk minor allele may be at least about ten years.

Serological Marker Association

Disclosed herein, in some embodiments are SNP is associated with the expression of serological markers. In some instances, a presence of one or more SNPs in a sample obtained from a subject is indicative that the subject has, or will develop, a disease or condition or subtype of the disease or condition, associated with a presence of a microbiome. Non-limiting examples of serological markers include anti-Saccharomyces cerevisiae (ASCA) anti-laminaribioside (ALCA), anti-chitobioside (ACCA), anti-mannobioside (AMCA), anti-laminarin (anti-L) and anti-chitin (anti-C), anti-outer membrane porin C (anti-OmpC), anti-Cbirl flagellin and anti-I2 antibody, and anti-neutrophil cytoplasmic autoantibodies (ANCA). In some instances, the association between a SNP and associated serological marker with an inflammatory disease or condition disclosed herein is stronger than the association between the SNP alone. In some instances, the presence of a serological marker in combination with the SNP is predictive of the inflammatory disease or condition.

In some instances, a SNP disclosed herein is associated with stricturing disease, penetrating disease, or a combination of stricturing and penetrating disease. Stricturing may be described as the presence of a stricture or narrowed region of the intestine. The stricture may comprise scar tissue. In some instances, a SNP disclosed herein is associated with penetrating. Penetrating may be described as the presence of a fistula. Fistulae may occur between sections of the bowel or between the bowel and skin. In some instances, the SNP is associated with stricturing, penetrating, and/or stricturing and penetrating disease is localized in the ileum, colon, or ileocolonic region of the intestine. In some instances, the SNP is associated with medically refractory disease, characterized by the failure of a standard treatment to induce remission of a disease in a subject. In some embodiments, the disease comprises an inflammatory disease disclosed herein. A non-limiting example of refractory inflammatory disease includes refractory Crohn's disease, and refractory ulcerative colitis (mrUC).

Expression Quantitative Trait Loci

Disclosed herein, in some embodiments, are genotypes comprising one or more SNPs associated with an increase or a decrease in eQTL expression. In some instances, the SNP occurs in an expression quantitative trait locus (eQTL). Expression quantitative trait loci are genomic loci that affect expression of an mRNA or protein. In some instances, a SNP in an eQTL results in increased IL18R1 expression. In some instances, a SNP in an eQTL results in decreased IL18R1 expression. In some instances, the eQTL is a local eQTL, e.g., within the gene locus. In some instances, the eQTL is a distant eQTL, e.g., outside of the gene locus. In some instances, the eQTL is on a different chromosome than the IL18R1 locus, referred to herein as a trans eQTL. In some instances, the eQTL is on the same chromosome as the IL18R1 locus, referred to herein as a cis eQTL. In some instances, the cis gene comprises a gene listed in FIG. 1A to FIG. 1QQQQQQQ.

In some instances, the cis gene comprises one or more of Mitogen-Activated Protein Kinase Kinase Kinase Kinase 4 (MAP4K4), Interleukin 1 Receptor Like 1 (IL1RL1), Transmembrane Protein 182 (TMEM182), and Interleukin 18 Receptor Accessory Protein (IL18RAP). In some instances, the SNP is associated with an increase in expression of MAP4K4. In some instances, the SNP is associated with an increase in expression of IL1RL1. In some instances, the SNP is associated with an increase in expression of TMEM182. In some instances, the SNP is associated with an increase in expression of IL18RAP. In some instances, the SNP is associated with an decrease in expression of MAP4K4. In some instances, the SNP is associated with an decrease in expression of IL1RL1. In some instances, the SNP is associated with an decrease in expression of TMEM182. In some instances, the SNP is associated with an decrease in expression of IL18RAP. In some instances the “increase” or the “decrease” is with reference to a level of the cis gene in a reference population. In some instances, the reference population is a “control” group of individuals who are not diseased.

In some instances, the eQTL is tissue-independent. In some instances, the eQTL is tissue-dependent. In some instances, methods disclosed herein comprise assaying for or detecting a SNP in an eQTL of rectum tissue. In some instances, methods disclosed herein comprise assaying for or detecting a SNP in an eQTL of anal tissue. In some instances, methods disclosed herein comprise assaying for or detecting a SNP in an eQTL of colon tissue. In some instances, methods disclosed herein comprising assaying for or detecting a SNP in an eQTL of the small intestine tissue. In some instances, methods disclosed herein comprise assaying for or detecting a SNP in an eQTL of intestinal tissue. In some instances, methods disclosed herein comprise assaying for or detecting a SNP in an eQTL of stomach tissue. In some instances, methods disclosed herein comprise assaying for or detecting a SNP in an eQTL of esophageal tissue. QTL mapping may be performed by analysis of variance (ANOVA), standard interval mapping, composite interval mapping, and family-based pedigree mapping. In some instances, the SNP is associated with an increase in expression of IL1RL1 in the small intestine tissue. In some instances, the SNP is associated with an increase in expression of MAP4K4 in the small intestine tissue. In some instances, the SNP is associated with an increase in expression of TMEM182in the small intestine tissue. In some instances, the SNP is associated with an increase in expression of IL18RAP in the small intestine tissue. In some instances, the SNP is associated with a decrease in expression of IL1RL1 in the small intestine tissue. In some instances, the SNP is associated with a decrease in expression of MAP4K4 in the small intestine tissue in the small intestine tissue. In some instances, the SNP is associated with a decrease in expression of TMEM182in the small intestine tissue. In some instances, the SNP is associated with a decrease in expression of IL18RAP in the small intestine tissue. In some instances, the SNP is associated with an increase in expression of IL1RL1 in the colon tissue. In some instances, the SNP is associated with an increase in expression of MAP4K4 in the colon tissue. In some instances, the SNP is associated with an increase in expression of TMEM182in the colon tissue. In some instances, the SNP is associated with an increase in expression of IL18RAP in the colon tissue. In some instances, the SNP is associated with a decrease in expression of IL1RL1 in the colon tissue. In some instances, the SNP is associated with a decrease in expression of MAP4K4 in the colon tissue in the colon tissue. In some instances, the SNP is associated with a decrease in expression of TMEM182in the colon tissue. In some instances, the SNP is associated with a decrease in expression of IL18RAP in the colon tissue.

The combination of an eQTL and an association between a SNP and an IBD may allow one to determine how gene expression is related to risk of disease. By way of non-limiting example, rs1921622 has both eQTL and an association with stricturing disease with evidence of penetrating disease in the colon of subjects with CD. eQTL shows that the major allele is associated with an upregulation (Cis_Beta=0.04961; eqtl_p value=0.01669) of the MAP4K4 mRNA in small bowel tissue and the association shows that major allele is associated with a risk of stricturing disease with evidence of penetrating disease in the colon of subjects with CD. In other cases the major or minor allele may code for downregulation of the gene and the minor allele might be the risk allele.

Method of Detection

Disclosed herein, in some embodiments, are methods of detecting a presence, absence, or level, of a genotype (e.g., IL18R1 risk genotype) or biomarker in a sample obtained from a subject. In some instances, the methods of detection disclosed herein are useful for the diagnosis, prognosis, monitoring of disease progression, selection for treatment, monitoring of treatment, and/or treatment of inflammatory bowel disease (e.g., Crohn's disease, ulcerative colitis, and the like) disclosed herein.

In some embodiments, methods of detecting a presence, absence, or level of a genotype or biomarker in the sample obtained from the subject involve detecting a nucleic acid sequence. In some cases, the nucleic acid sequence comprises deoxyribonucleic acid (DNA). In some instances, the nucleic acid sequence comprises a denatured DNA molecule or fragment thereof. In some instances, the nucleic acid sequence comprises DNA selected from: genomic DNA, viral DNA, mitochondrial DNA, plasmid DNA, amplified DNA, circular DNA, circulating DNA, cell-free DNA, or exosomal DNA. In some instances, the DNA is single-stranded DNA (ssDNA), double-stranded DNA, denaturing double-stranded DNA, synthetic DNA, and combinations thereof. The circular DNA may be cleaved or fragmented. In some instances, the nucleic acid sequence comprises ribonucleic acid (RNA). In some instances, the nucleic acid sequence comprises fragmented RNA. In some instances, the nucleic acid sequence comprises partially degraded RNA. In some instances, the nucleic acid sequence comprises a microRNA or portion thereof. In some instances, the nucleic acid sequence comprises an RNA molecule or a fragmented RNA molecule (RNA fragments) selected from: a microRNA (miRNA), a pre-miRNA, a pri-miRNA, a mRNA, a pre-mRNA, a viral RNA, a viroid RNA, a virusoid RNA, circular RNA (circRNA), a ribosomal RNA (rRNA), a transfer RNA (tRNA), a pre-tRNA, a long non-coding RNA (lncRNA), a small nuclear RNA (snRNA), a circulating RNA, a cell-free RNA, an exosomal RNA, a vector-expressed RNA, an RNA transcript, a synthetic RNA, and combinations thereof.

Disclosed herein, in some embodiments, the genotype or biomarker is detected by subjecting a sample obtained from the subject to a nucleic acid-based detection assay. In some instances, the nucleic acid-based detection assay comprises quantitative polymerase chain reaction (qPCR), gel electrophoresis (including for e.g., Northern or Southern blot), immunochemistry, in situ hybridization such as fluorescent in situ hybridization (FISH), cytochemistry, or sequencing. In some embodiments, the sequencing technique comprises next generation sequencing. In some embodiments, the methods involve a hybridization assay such as fluorogenic qPCR (e.g., TaqMan™, SYBR green, SYBR green I, SYBR green II, SYBR gold, ethidium bromide, methylene blue, Pyronin Y, DAPI, acridine orange, Blue View or phycoerythrin), which involves a nucleic acid amplification reaction with a specific primer pair, and hybridization of the amplified nucleic acid probes comprising a detectable moiety or molecule that is specific to a target nucleic acid sequence. In some instances, a number of amplification cycles for detecting a target nucleic acid in a qPCR assay is about 5 to about 30 cycles. In some instances, the number of amplification cycles for detecting a target nucleic acid is at least about 5 cycles. In some instances, the number of amplification cycles for detecting a target nucleic acid is at most about 30 cycles. In some instances, the number of amplification cycles for detecting a target nucleic acid is about 5 to about 10, about 5 to about 15, about 5 to about 20, about 5 to about 25, about 5 to about 30, about 10 to about 15, about 10 to about 20, about 10 to about 25, about 10 to about 30, about 15 to about 20, about 15 to about 25, about 15 to about 30, about 20 to about 25, about 20 to about 30, or about 25 to about 30 cycles. For TaqMan™ methods, the probe may be a hydrolysable probe comprising a fluorophore and quencher that is hydrolyzed by DNA polymerase when hybridized to a target nucleic acid. In some cases, the presence of a target nucleic acid is determined when the number of amplification cycles to reach a threshold value is less than 30, 29, 28, 27, 26, 25, 24, 23, 22, 21, or 20 cycles. In some instances, hybridization may occur at standard hybridization temperatures, e.g., between about 35° C. and about 65° C. in a standard PCR buffer.

An additional exemplary nucleic acid-based detection assay comprises the use of nucleic acid probes conjugated or otherwise immobilized on a bead, multi-well plate, or other substrate, wherein the nucleic acid probes are configured to hybridize with a target nucleic acid sequence. In some instances, the nucleic acid probe is specific to one or more genetic variants disclosed herein is used. In some instances, the nucleic acid probe specific to a SNP or SNV comprises a nucleic acid probe sequence sufficiently complementary to a risk or protective allele of interest, such that hybridization is specific to the risk or protective allele. In some instances, the nucleic acid probe specific to an indel comprises a nucleic acid probe sequence sufficiently complementary to an insertion of a nucleobase within a polynucleotide sequence flanking the insertion, such that hybridization is specific to the indel. In some instances, the nucleic acid probe specific to an indel comprises a probe sequence sufficiently complementary to a polynucleotide sequence flanking a deletion of a nucleobase within the polynucleotide sequence, such that hybridization is specific to the indel. In some instances, the nucleic acid probe specific to a biomarker comprises a nucleic acid probe sequence sufficiently complementary to the polynucleotide sequence of the biomarker. In some instances, the biomarker comprises a transcribed polynucleotide sequence (e.g., RNA, cDNA). In some embodiments, the nucleic acid probe can be, for example, a full-length cDNA, or a portion thereof, such as an oligonucleotide of at least about 7, 8, 9, 10, 11, 12, 13, 14, 15, 20, 25, 30, 35, 40, 45, or 50 nucleotides in length and sufficient to specifically hybridize under standard hybridization conditions to the target nucleic acid sequence. In some embodiments, the target nucleic acid sequence is immobilized on a solid surface and contacted with a probe, for example by running the isolated target nucleic acid sequence on an agarose gel and transferring the target nucleic acid sequence from the gel to a membrane, such as nitrocellulose. In some embodiments, the probe(s) are immobilized on a solid surface, for example, in an Affymetrix gene chip array, and the probe(s) are contacted with the target nucleic acid sequence. The present disclosure provides exemplary probes that are hybridizable to a target nucleic acid sequence comprising one or more single nucleotide polymorphisms (SNPS) at rs13001325, rs1420101, rs12479210, rs950880, rs13020553, rs13019081, rs12712141, rs2287037, rs1420102, rs12466380, rs1997467, rs1558619, rs1420088, rs12999364, rs4142132, rs12987977, rs11690443, rs1362350, rs12996505, rs873022, rs974389, rs3771177, rs3732129, rs17026974, rs6706844, rs13020793, rs11685480, rs1558622, rs10183388, rs12712135, rs10189711, rs11685424, rs10189202, rs10191914, rs11123918, rs1968171, rs6733174, rs59247511, rs1558620, rs1921622, rs12998521, rs13017455, rs1362349, rs11123923, rs10190555, rs1035127, rs17027087, rs2080289, rs4851570, rs17027060, rs12712145, rs1420098, rs3732123, rs2287034, rs3860444, rs3821203, rs56258475, rs2270298, rs4851006, rs6710885, rs1568681, rs2241117, rs17027037, rs2270297, rs6753717, rs3755274, rs17027071, rs6750020, rs17027006, rs11683700, rs2058622, rs4851007, rs3732126, rs1807782, rs12469506, rs4851575, rs3771172, rs11465633, rs1135354, rs1558627, rs55927292, rs3771171, rs13015714, rs2160202, rs55883125, rs2041740, rs1035130, rs1420103, rs67723747, rs6543116, rs55664618, rs4851005, rs17027056, rs1420089, rs62152661, rs1420095, rs56030066, rs62152714, rs17696376, rs12105808, rs78248680, rs56151044, rs62152662, rs17651485, rs3771170, rs11123926, rs76721133, rs4988955, rs9807962, rs9808453, rs13424006, rs11695627, rs3771166, rs10173193, rs11465575, rs4851566, rs9308857, rs1974675, rs6751967, rs3771162, rs56386507, rs1997466, rs12712140, and/or rs1362348. In some instances, the probe comprises at least about 10 nucleic acids within SEQ ID NOS: 1-7, or reverse complement thereof, including the nucleobase indicated with an “N”. In some embodiments, the “N” within SEQ ID NO: 1 comprises an “A” or a “G.” In some embodiments, the “N” within SEQ ID NO: 2 comprises an “A” or a “G.” In some embodiments, the “N” within SEQ ID NO: 3 comprises a “C” or a “T.” In some embodiments, the “N” within SEQ ID NO: 4 comprises an “A” or a “G.” In some embodiments, the “N” within SEQ ID NO: 5 comprises an “A” or a “G.” In some embodiments, the “N” within SEQ ID NO: 6 comprises an “A” or a “G.” In some embodiments, the “N” within SEQ ID NO: 7 comprises an “A” or a “G.”

In some embodiments, the term “probe” with regards to nucleic acids, refers to any nucleic acid molecule that is capable of selectively binding to a specifically intended target nucleic acid sequence. In some instances, probes are specifically designed to be labeled, for example, with a radioactive label, a fluorescent label, an enzyme, a chemiluminescent tag, a colorimetric tag, or other labels or tags that are known in the art. In some instances, the fluorescent label comprises a fluorophore. In some instances, the fluorophore is an aromatic or heteroaromatic compound. In some instances, the fluorophore is a pyrene, anthracene, naphthalene, acridine, stilbene, benzoxaazole, indole, benzindole, oxazole, thiazole, benzothiazole, canine, carbocyanine, salicylate, anthranilate, xanthenes dye, coumarin. Exemplary xanthene dyes include, e.g., fluorescein and rhodamine dyes. Fluorescein and rhodamine dyes include, but are not limited to 6-carboxyfluorescein (FAM), 2′7′-dimethoxy-4′5′-dichloro-6-carboxyfluorescein (JOE), tetrachlorofluorescein (TET), 6-carboxyrhodamine (R6G), N,N,N; N′-tetramethyl-6-carboxyrhodamine (TAMRA), 6-carboxy-X-rhodamine (ROX). Suitable fluorescent probes also include the naphthylamine dyes that have an amino group in the alpha or beta position. For example, naphthylamino compounds include 1-dimethylaminonaphthyl-5-sulfonate, 1-anilino-8-naphthalene sulfonate and 2-p-toluidinyl-6-naphthalene sulfonate, 5-(2′-aminoethyl)aminonaphthalene-1-sulfonic acid (EDANS). Exemplary coumarins include, e.g., 3-phenyl-7-isocyanatocoumarin; acridines, such as 9-isothiocyanatoacridine and acridine orange; N-(p-(2-benzoxazolyl)phenyl) maleimide; cyanines, such as, e.g., indodicarbocyanine 3 (Cy3), indodicarbocyanine 5 (Cy5), indodicarbocyanine 5.5 (Cy5.5), 3-(−carboxy-pentyl)-3′-ethyl-5,5′-dimethyloxacarbocyanine (CyA); 1H, 5H, 11H, 15H-Xantheno[2,3,4-ij: 5,6,7-i′j′]diquinolizin-18-ium, 9-[2 (or 4)-[[[6-[2,5-dioxo-1-pyrrolidinyl)oxy]-6-oxohexyl]amino]sulfonyl]-4 (or 2)-sulfophenyl]-2,3, 6,7, 12,13, 16,17-octahydro-inner salt (TR or Texas Red); or BODIPY™ dyes. In some cases, the probe comprises FAM as the dye label.

Disclosed herein, in some embodiments, a genotype or biomarker is detected by subjecting a sample obtained from the subject to a nucleic acid amplification assay. In some instances, the amplification assay comprises polymerase chain reaction (PCR), qPCR, self-sustained sequence replication, transcriptional amplification system, Q-Beta Replicase, rolling circle replication, or any suitable other nucleic acid amplification technique. A suitable nucleic acid amplification technique is configured to amplify a region of a nucleic acid sequence comprising one or more genetic risk variants disclosed herein. In some instances, the amplification assays requires primers. The nucleic acid sequence for the genetic risk variants and/or genes known or provided herein is sufficient to enable one of skill in the art to select primers to amplify any portion of the gene or genetic variants. A DNA sample suitable as a primer may be obtained, e.g., by polymerase chain reaction (PCR) amplification of genomic DNA, fragments of genomic DNA, fragments of genomic DNA ligated to adaptor sequences or cloned sequences. A person of skill in the art would utilize computer programs to design of primers with the desired specificity and optimal amplification properties, such as Oligo version 7.0 (National Biosciences). Controlled robotic systems are useful for isolating and amplifying nucleic acids and can be used.

In some embodiments, detecting the biomarker or genotype of the subject comprises sequencing genetic material obtained from a biological sample from the subject. Sequencing can be performed with any appropriate sequencing technology, including but not limited to single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis. Sequencing methods also include next-generation sequencing, e.g., modern sequencing technologies such as Illumina sequencing (e.g., Solexa), Roche 454 sequencing, Ion torrent sequencing, and SOLiD sequencing. In some cases, next-generation sequencing involves high-throughput sequencing methods. Additional sequencing methods available to one of skill in the art may also be employed.

In some instances, a number of nucleotides that are sequenced are at least 5, 10, 15, 20, 25, 30, 35, 40, 45, 50, 100, 150, 200, 300, 400, 500, 2000, 4000, 6000, 8000, 10000, 20000, 50000, 100000, or more than 100000 nucleotides. In some instances, the number of nucleotides sequenced is in a range of about 1 to about 100000 nucleotides, about 1 to about 10000 nucleotides, about 1 to about 1000 nucleotides, about 1 to about 500 nucleotides, about 1 to about 300 nucleotides, about 1 to about 200 nucleotides, about 1 to about 100 nucleotides, about 5 to about 100000 nucleotides, about 5 to about 10000 nucleotides, about 5 to about 1000 nucleotides, about 5 to about 500 nucleotides, about 5 to about 300 nucleotides, about 5 to about 200 nucleotides, about 5 to about 100 nucleotides, about 10 to about 100000 nucleotides, about 10 to about 10000 nucleotides, about 10 to about 1000 nucleotides, about 10 to about 500 nucleotides, about 10 to about 300 nucleotides, about 10 to about 200 nucleotides, about 10 to about 100 nucleotides, about 20 to about 100000 nucleotides, about 20 to about 10000 nucleotides, about 20 to about 1000 nucleotides, about 20 to about 500 nucleotides, about 20 to about 300 nucleotides, about 20 to about 200 nucleotides, about 20 to about 100 nucleotides, about 30 to about 100000 nucleotides, about 30 to about 10000 nucleotides, about 30 to about 1000 nucleotides, about 30 to about 500 nucleotides, about 30 to about 300 nucleotides, about 30 to about 200 nucleotides, about 30 to about 100 nucleotides, about 50 to about 100000 nucleotides, about 50 to about 10000 nucleotides, about 50 to about 1000 nucleotides, about 50 to about 500 nucleotides, about 50 to about 300 nucleotides, about 50 to about 200 nucleotides, or about 50 to about 100 nucleotides.

Disclosed herein, in some embodiments, are methods for detecting a transcriptomic risk signature or transcriptomic risk profile in a sample obtained from the subject. In some embodiments, the presence, level, or activity of two or more biomarkers in a sample is determined by detecting a transcribed or reverse transcribed polynucleotide, or portion thereof (e.g., mRNA, or cDNA), of a target gene making up the transcriptomic risk signature or transcriptomic risk profile. Any suitable method of detecting a biomarker, such as those disclosed herein, may be utilized to detect a transcriptomic risk signature or transcriptomic risk profile, such as those disclosed herein. A transcriptomic risk signature or transcriptomic risk profile can also be detected at the protein level, using a detection reagent that detects the protein product encoded by the mRNA of the biomarker, directly or indirectly, such the detection reagents disclosed herein.

Disclosed herein, in some embodiments, genetic material is extracted from a sample obtained from a subject, e.g., a sample of blood or serum. In certain embodiments where nucleic acids are extracted, the nucleic acids are extracted using any technique that does not interfere with subsequent analysis. In certain embodiments, this technique uses alcohol precipitation using ethanol, methanol or isopropyl alcohol. In certain embodiments, this technique uses phenol, chloroform, or any combination thereof. In certain embodiments, this technique uses cesium chloride. In certain embodiments, this technique uses sodium, potassium or ammonium acetate or any other salt commonly used to precipitate DNA. In certain embodiments, this technique utilizes a column or resin based nucleic acid purification scheme such as those commonly sold commercially, one non-limiting example would be the GenElute Bacterial Genomic DNA Kit available from Sigma Aldrich. In certain embodiments, after extraction the nucleic acid is stored in water, Tris buffer, or Tris-EDTA buffer before subsequent analysis. In an exemplary embodiment, the nucleic acid material is extracted in water. In some cases, extraction does not comprise nucleic acid purification. In certain embodiments, RNA may be extracted from cells using RNA extraction techniques including, for example, using acid phenol/guanidine isothiocyanate extraction (RNAzol B; Biogenesis), RNeasy RNA preparation kits (Qiagen) or PAXgene (PreAnalytix, Switzerland).

In some embodiments, methods of detecting a presence, absence, or level of a target protein (e.g., biomarker) in the sample obtained from the subject involve detecting protein activity or expression. A target protein may be detected by use of an antibody-based assay, where an antibody specific to the target protein is utilized. In some embodiments, antibody-based detection methods utilize an antibody that binds to any region of target protein. An exemplary method of analysis comprises performing an enzyme-linked immunosorbent assay (ELISA). The ELISA assay may be a sandwich ELISA or a direct ELISA. Another exemplary method of analysis comprises a single molecule array, e.g., Simoa. Other exemplary methods of detection include immunohistochemistry and lateral flow assay. Additional exemplary methods for detecting target protein include, but are not limited to, gel electrophoresis, capillary electrophoresis, high performance liquid chromatography (HPLC), thin layer chromatography (TLC), hyperdiffusion chromatography, and the like, or various immunological methods such as fluid or gel precipitation reactions, immunodiffusion (single or double), immunoelectrophoresis, radioimmunoassay (RIA), immunofluorescent assays, and Western blotting. In some embodiments, antibodies, or antibody fragments, are used in methods such as Western blots or immunofluorescence techniques to detect the expressed proteins. The antibody or protein can be immobilized on a solid support for Western blots and immunofluorescence techniques. Suitable solid phase supports or carriers include any support capable of binding an antigen or an antibody. Exemplary supports or carriers include glass, polystyrene, polypropylene, polyethylene, dextran, nylon, amylases, natural and modified celluloses, polyacrylamides, gabbros, and magnetite.

In some cases, a target protein may be detected by detecting binding between the target protein and a binding partner of the target protein. In some cases, the target protein comprises IL18R1. Non-limiting examples of binding partners of IL18R1 include IL18. Exemplary methods of analysis of protein-protein binding comprise performing an assay in vivo or in vitro, or ex vivo. In some instances, the method of analysis comprises an assay such as a co-immunoprecipitation (co-IP), pull-down, crosslinking protein interaction analysis, labeled transfer protein interaction analysis, or Far-western blot analysis, FRET based assay, including, for example FRET-FLIM, a yeast two-hybrid assay, BiFC, or split luciferase assay.

Disclosed herein, in some embodiments, are methods of detecting a presence or a level of one or more serological markers in a sample obtained from a subject. In some embodiments, the one or more serological markers comprises comprise anti-Saccharomyces cerevisiae antibody (ASCA), an anti-neutrophil cytoplasmic antibody (ANCA), E. coli outer membrane porin protein C (OmpC), anti-Malassezia restricta antibody, anti-Malassezia pachydermatis antibody, anti-Malassezia furfur antibody, anti-Malassezia globasa antibody, anti-Cladosporium albicans antibody, anti-laminaribiose antibody (ALCA), anti-chitobioside antibody (ACCA), anti-laminarin antibody, anti-chitin antibody, pANCA antibody, anit-I2 antibody, and anti-Cbirl flagellin antibody. In some embodiments, the antibodies comprises immunoglobulin A (IgA), immunoglobulin G (IgG), immunoglobulin E (IgE), or immunoglobulin M (IgM), immunoglobulin D (IgD), or a combination thereof. Any suitable method for detecting a target protein or biomarker disclosed herein may be used to detect a presence, absence, or level of a serological marker. In some embodiments, the presence or the level of the one or more serological markers is detected using an enzyme-linked immunosorbent assay (ELISA), a single molecule array (Simoa), immunohistochemistry, internal transcribed spacer (ITS) sequencing, or any combination thereof. In some embodiments, the ELISA is a fixed leukocyte ELISA. In some embodiments, the ELISA is a fixed neutrophil ELISA. A fixed leukocyte or neutrophil ELISA may be useful for the detection of certain serological markers, such as those described in Saxon et al., A distinct subset of antineutrophil cytoplasmic antibodies is associated with inflammatory bowel disease, J. Allergy Clin. Immuno. 86:2; 202-210 (August 1990). In some embodiments, ELISA units (EU) are used to measure positivity of a presence or level of a serological marker (e.g., seropositivity), which reflects a percentage of a standard or reference value. In some embodiments, the standard comprises pooled sera obtained from well-characterized patient population (e.g., diagnosed with the same disease or condition the subject has, or is suspected of having) reported as being seropositive for the serological marker of interest. In some embodiments, the control or reference value comprises 10, 20, 30, 40, 50, 60, 70, 80, 90, or 100 EU. In some instances, a quartile sum scores are calculated using, for example, the methods reported in Landers C J, Cohavy O, Misra R. et al., Selected loss of tolerance evidenced by Crohn's disease-associated immune responses to auto- and microbial antigens. Gastroenterology (2002)123:689-699.

Methods of Diagnosis and Prognosis

Disclosed herein, in some embodiments, are methods of diagnosing a disease or condition in a subject. In some cases, the disease or condition comprises an inflammatory disease, fibrostenotic disease, and/or fibrotic disease. Non-limiting examples of inflammatory diseases include diseases of the GI tract, liver, gallbladder, and joints. In some cases, the inflammatory disease IBD, CD, UC, systemic lupus erythematosus (SLE), or rheumatoid arthritis. In some embodiments, the disease or condition comprises fibrosis, fibrostenosis, or a fibrotic disease, either isolated or in combination with an inflammatory disease. An exemplary fibrotic disease is PSC. In some embodiments, a subtype of the disease or condition is diagnosed in the subject. Non-limiting examples of subtypes of IBD include, stricturing disease, penetrating disease, stricturing and penetrating disease, obstructive disease, refractory disease, or another complicated form of IBD. In some instances, the subject is diagnosed with, or predicted to develop, one disease or condition, two disease or conditions, three disease or conditions, or more.

Disclosed herein, in some embodiments, are methods of diagnosing a disease or condition in a subject comprising: (a) obtaining a sample from a subject; (b) subjecting the sample to an assay configured to detect a presence, absence, or level, of one or more IL18R1 risk genotypes; (c) diagnosing the subject with the disease or condition, provided the presence, absence, or level of one or more IL18R1 risk genotypes is detected in the sample obtained from the subject. In some embodiments, the one or more IL18R1 risk genotypes is detected using one or more methods of detection, kits and/or compositions disclosed herein. In some embodiments, the subject is treated by administering a therapeutically effective amount of a therapeutic agent and/or additional agent disclosed herein to the subject, provided the subject is diagnosed with the disease or condition. In some embodiments, the therapeutic agent comprises an antagonist of IL18R1. In some embodiments, the one or more IL18R1 risk genotypes is associated with Crohn's disease. In some embodiments, the one or more IL18R1 risk genotypes is associated with inflammatory bowel disease. In some embodiments, the one or more IL18R1 risk genotypes is associated with ulcerative colitis.

Disclosed herein, in some embodiments, are methods of predicting whether a subject will develop a disease or condition, the method comprising: (a) obtaining a sample from a subject; (b) subjecting the sample to an assay configured to detect a presence, absence, or level, of one or more IL18R1 risk genotypes; (c) predicting that the subject will develop the disease or condition, provided the presence, absence, or level of the one or more IL18R1 risk genotypes is detected in the sample obtained from the subject. In some embodiments, the one or more IL18R1 risk genotypes is detected using one or more methods of detection, kits and/or compositions disclosed herein. In some embodiments, the subject is treated by administering a therapeutically effective amount of a therapeutic agent and/or additional agent disclosed herein to the subject, provided the subject is predicted to develop the disease or condition. In some embodiments, the therapeutic agent comprises an antagonist of IL18R1. In some embodiments, the one or more IL18R1 risk genotypes is associated with Crohn's disease. In some embodiments, the one or more IL18R1 risk genotypes is associated with inflammatory bowel disease. In some embodiments, the one or more IL18R1 risk genotypes is associated with ulcerative colitis.

In some embodiments, the one or more IL18R1 risk genotypes comprises a SNP provided in FIG. 1A to FIG. 1QQQQQQQ. In some instances, the one or more IL18R1 risk genotypes is provided in Tables 1-5. In some instances, the one or more SNPs of IL18R1 comprise rs13001325, rs1420101, rs12479210, rs950880, rs13020553, rs13019081, rs12712141, rs2287037, rs1420102, rs12466380, rs1997467, rs1558619, rs1420088, rs12999364, rs4142132, rs12987977, rs11690443, rs1362350, rs12996505, rs873022, rs974389, rs3771177, rs3732129, rs17026974, rs6706844, rs13020793, rs11685480, rs1558622, rs10183388, rs12712135, rs10189711, rs11685424, rs10189202, rs10191914, rs11123918, rs1968171, rs6733174, rs59247511, rs1558620, rs1921622, rs12998521, rs13017455, rs1362349, rs11123923, rs10190555, rs1035127, rs17027087, rs2080289, rs4851570, rs17027060, rs12712145, rs1420098, rs3732123, rs2287034, rs3860444, rs3821203, rs56258475, rs2270298, rs4851006, rs6710885, rs1568681, rs2241117, rs17027037, rs2270297, rs6753717, rs3755274, rs17027071, rs6750020, rs17027006, rs11683700, rs2058622, rs4851007, rs3732126, rs1807782, rs12469506, rs4851575, rs3771172, rs11465633, rs1135354, rs1558627, rs55927292, rs3771171, rs13015714, rs2160202, rs55883125, rs2041740, rs1035130, rs1420103, rs67723747, rs6543116, rs55664618, rs4851005, rs17027056, rs1420089, rs62152661, rs1420095, rs56030066, rs62152714, rs17696376, rs12105808, rs78248680, rs56151044, rs62152662, rs17651485, rs3771170, rs11123926, rs76721133, rs4988955, rs9807962, rs9808453, rs13424006, rs11695627, rs3771166, rs10173193, rs11465575, rs4851566, rs9308857, rs1974675, rs6751967, rs3771162, rs56386507, rs1997466, rs12712140, and/or rs1362348, or a SNP in linkage disequilibrium therewith.

Methods of Characterizing a Subtype of a Disease or Condition

Disclosed herein, in some embodiments, are methods of characterizing a disease or condition, or a subtype of a disease or condition. In some cases, the disease or condition comprises an inflammatory disease, fibrostenotic disease, and/or fibrotic disease. Non-limiting examples of inflammatory diseases include diseases of the GI tract, liver, gallbladder, and joints. In some cases, the inflammatory disease IBD, CD, UC, systemic lupus erythematosus (SLE), or rheumatoid arthritis. In some embodiments, the disease or condition comprises fibrosis, fibrostenosis, or a fibrotic disease, either isolated or in combination with an inflammatory disease. An exemplary fibrotic disease is PSC. Non-limiting examples of subtypes of IBD include, stricturing disease, penetrating disease, stricturing and penetrating disease, obstructive disease, refractory disease, or another complicated form of IBD.

Disclosed herein, in some embodiments, are methods of characterizing a disease or condition, or a subtype of a disease or condition comprising: (a) obtaining a sample from a subject; (b) subjecting the sample to an assay configured to detect a presence, absence, or level, of one or more IL18R1 risk genotypes; (c) characterizing the disease or condition as being associated with at least one of non-stricturing and non-penetrating, stricturing, and penetrating, provided the presence, absence, or level of one or more IL18R1 risk genotypes is detected in the sample obtained from the subject. In some embodiments, the one or more IL18R1 risk genotypes is detected using one or more methods of detection, kits and/or compositions disclosed herein. In some embodiments, the subject is treated by administering a therapeutically effective amount of a therapeutic agent and/or additional agent disclosed herein to the subject, provided the subject is disease or condition is characterized as being associated with at least one of non-stricturing and non-penetrating, stricturing, and penetrating. In some embodiments, the therapeutic agent comprises a modulator of IL18R1.

Disclosed herein, in some embodiments, are methods of characterizing a disease or condition, or a subtype of a disease or condition comprising: (a) obtaining a sample from a subject; (b) subjecting the sample to an assay configured to detect a presence, absence, or level, of one or more IL18R1 risk genotypes; (c) characterizing the disease or condition as being associated with at least one of non-stricturing and non-penetrating, stricturing, and penetrating that is isolated to an ileum, ileocolonic region of an intestine, or colon, provided the presence, absence, or level of one or more IL18R1 risk genotypes is detected in the sample obtained from the subject. In some embodiments, the one or more IL18R1 risk genotypes is detected using one or more methods of detection, kits and/or compositions disclosed herein. In some embodiments, the subject is treated by administering a therapeutically effective amount of a therapeutic agent and/or additional agent disclosed herein to the subject, provided the subject is disease or condition is characterized as being associated with the at least one of non-stricturing and non-penetrating, stricturing, and penetrating is isolated to an ileum, ileocolonic region of an intestine, or colon. In some embodiments, the therapeutic agent comprises a modulator of IL18R1.

Disclosed herein, in some embodiments, are methods of characterizing a disease or condition, or a subtype of a disease or condition comprising: (a) obtaining a sample from a subject; (b) subjecting the sample to an assay configured to detect a presence, absence, or level, of one or more IL18R1 risk genotypes; (c) characterizing the disease or condition as being associated with morphological defects in ileal Paneth cells, provided the presence, absence, or level of one or more IL18R1 risk genotypes is detected in the sample obtained from the subject. In some embodiments, the one or more IL18R1 risk genotypes is detected using one or more methods of detection, kits and/or compositions disclosed herein. In some embodiments, the subject is treated by administering a therapeutically effective amount of a therapeutic agent and/or additional agent disclosed herein to the subject, provided the subject is disease or condition is characterized as being associated with morphological defects in ileal Paneth cells. In some embodiments, the therapeutic agent comprises an modulator of IL18R1.

In some embodiments, the one or more IL18R1 risk genotypes comprises a SNP provided in FIG. 1A to FIG. 1QQQQQQQ. In some instances, the one or more IL18R1 risk genotypes is provided in Tables 1-5. In some instances, the one or more SNPs of IL18R1 comprise rs13001325, rs1420101, rs12479210, rs950880, rs13020553, rs13019081, rs12712141, rs2287037, rs1420102, rs12466380, rs1997467, rs1558619, rs1420088, rs12999364, rs4142132, rs12987977, rs11690443, rs1362350, rs12996505, rs873022, rs974389, rs3771177, rs3732129, rs17026974, rs6706844, rs13020793, rs11685480, rs1558622, rs10183388, rs12712135, rs10189711, rs11685424, rs10189202, rs10191914, rs11123918, rs1968171, rs6733174, rs59247511, rs1558620, rs1921622, rs12998521, rs13017455, rs1362349, rs11123923, rs10190555, rs1035127, rs17027087, rs2080289, rs4851570, rs17027060, rs12712145, rs1420098, rs3732123, rs2287034, rs3860444, rs3821203, rs56258475, rs2270298, rs4851006, rs6710885, rs1568681, rs2241117, rs17027037, rs2270297, rs6753717, rs3755274, rs17027071, rs6750020, rs17027006, rs11683700, rs2058622, rs4851007, rs3732126, rs1807782, rs12469506, rs4851575, rs3771172, rs11465633, rs1135354, rs1558627, rs55927292, rs3771171, rs13015714, rs2160202, rs55883125, rs2041740, rs1035130, rs1420103, rs67723747, rs6543116, rs55664618, rs4851005, rs17027056, rs1420089, rs62152661, rs1420095, rs56030066, rs62152714, rs17696376, rs12105808, rs78248680, rs56151044, rs62152662, rs17651485, rs3771170, rs11123926, rs76721133, rs4988955, rs9807962, rs9808453, rs13424006, rs11695627, rs3771166, rs10173193, rs11465575, rs4851566, rs9308857, rs1974675, rs6751967, rs3771162, rs56386507, rs1997466, rs12712140, and/or rs1362348, or a SNP in linkage disequilibrium therewith

Methods of Treatment

Disclosed herein, in some embodiments, are methods of treating a disease or condition, or a symptom of the disease or condition, in a subject, comprising administrating of therapeutic effective amount of one or more therapeutic agents to the subject. In some embodiments, the one or more therapeutic agents is administered to the subject alone (e.g., standalone therapy). In some embodiments, the one or more therapeutic agents is administered in combination with an additional agent. In some embodiments, the therapeutic agent is a first-line therapy for the disease or condition. In some embodiments, the therapeutic agent is a second-line, third-line, or fourth-line therapy, for the disease or condition.

Therapeutic Agent

Disclosed herein, in some embodiments, are therapeutic agents useful for the treatment of a disease or condition, or symptom of the disease or condition, disclosed herein. In some embodiments, the therapeutic agent comprises a modulator, agonist, and/or antagonist of interleukin 18 receptor 1 (IL18R1).

Methods disclosed herein may comprise and/or utilize a therapeutic agent or use thereof, wherein the therapeutic agent is effective to modify expression and/or activity of IL18R1 (e.g., modulator of IL18R1). Therapeutic agents that modify expression and/or activity of IL18R1 may also be referred to herein as IL18R1-targeting agents. Alternatively or additionally, compositions, kits and methods disclosed herein may comprise and/or utilize a therapeutic agent or use thereof, wherein the therapeutic agent modifies expression and/or activity of a protein that functions upstream or downstream of a pathway that involves IL18R1. In some embodiments, the modulator of IL18R1 is effective to increase or activate the activity or expression of IL18R1 in the subject (e.g., agonist or partial agonist). In some embodiments, the modulator of IL18R1 is effective to decrease or reduce the activity or expression of IL18R1 (e.g., antagonist or partial antagonist).

In some embodiments, the IL18R1 modulator is an antibody, an antigen binding fragment, a RNA interfering agent (RNAi), a small interfering RNA (siRNA), a short hairpin RNA (shRNA), a microRNA (miRNA), an antisense oligonucleotide, a peptide, a peptidomimetic, a small molecule, or an aptamer.

In some instances, the therapeutic agent is an antagonist of IL18R1. In some instances, the antagonist acts as an inverse agonist. In some instances, the therapeutic agent is an allosteric modulator of IL18R1. Methods disclosed herein may comprise administering IL18R1-targeting agents alone. In other instances, methods disclosed herein may comprise administering IL18R1-targeting agents along with another therapeutic agent disclosed herein, a nutritional-based therapy, a nature-based therapy, a diet-based therapy, or a combination thereof.

In some instances, the subject has a SNP that is associated with, or causes, an increased expression of IL18R1. In some instances, the subject has a SNP that is associated with, or causes increased activity of IL18R1. In some instances, the SNP is associated with, or causes and increase expression of IL18R1. In some instances, the SNP is associated with, or causes an increase activity of IL18R1. In these instances, it may be suitable to use an IL18R1 antagonist to bring IL18R1 activity back to a normal level, e.g., that of a person without the IBD of the subject.

In some instances, the subject has a SNP that is associated with, or causes decreased expression of IL18R1. In some instances, the subject has a SNP is associated with, or causes, decreased activity of IL18R1. In some instances, the SNP is associated with, or causes, a decrease in expression of IL18R1. In some instances, the SNP is associated with, or causes, decreased activity of IL18R1. In these instances, it may be suitable to use an IL18R1 agonist to bring IL18R1 activity back to a normal level, e.g., that of a person without the IBD of the subject.

In some instances, the therapeutic agent is a small molecule drug. By way of non-limiting example, a small molecule drug may be a chemical compound. In some instances, the therapeutic agent is a large molecule drug. Large molecule drugs generally comprise a peptide or nucleic acid. By way of non-limiting example, the large molecule drug may comprise an antibody or antigen binding antibody fragment. In some instances, the therapeutic agent comprises a small molecule and a large molecule. By way of non-limiting example, the therapeutic agent may comprise an antibody-drug conjugate.

In some instances, the therapeutic agent is a small molecule that binds IL18R1. In some instances, the small molecule that binds IL18R1 is an IL18R1 agonist. In some instances, the small molecule that binds IL18R1 is an IL18R1 partial agonist. In some instances, the small molecule that binds IL18R1 is an IL18R1 antagonist. In some instances, the small molecule that binds IL18R1 is an IL18R1 partial agonist.

In some instances, the therapeutic agent is a modulator of IL18R1 binding protein. For example, the therapeutic agent is a modulator of IL18. In some instances, the modulator of IL18 is an antibody, an antigen binding fragment, a RNA interfering agent (RNAi), a small interfering RNA (siRNA), a short hairpin RNA (shRNA), a microRNA (miRNA), an antisense oligonucleotide, a peptide, a peptidomimetic, a small molecule, or an aptamer.

In some embodiments, the agonist of IL18R1 comprises an IL18R1 polypeptide. In some embodiments, the IL18R1 polypeptide comprises a human IL18R1 protein (huIL18R1), or a homolog thereof. In some instances the polypeptide is an antagonist, agonist or modulator (e.g., allosteric modulator, orthosteric modulator) of IL18R1. In some embodiments, the IL18R1 polypeptide comprises a recombinant IL18R1 polypeptide. In some embodiments, the recombinant huIL18R1 precursor protein comprises SEQ ID NO: 8)(MNCRELPLTLWVLISVSTAESCTSRPHITVVEGEPFYLKHCSCSLAHEIETTYKSWYKSSGSQE HVELNPRSSSRIALHDCVLEFWPVELNDTGSYFFQMKNYTQKWKLNVIRRNKHSCFTERQVT SKIVEVKKFFQITCENSYYQTLVNSTSLYKNCKKLLLENNKNPTIKKNAEFEDQGYYSCVHFL HHNGKLFNITKTFNMVEDRSNIVPVLLGPKLNHVAVELGKNVRLNCSALLNEEDVIYWMFG EENGSDPNIHEEKEMRIMTPEGKWHASKVLRIENIGESNLNVLYNCTVASTGGTDTKSFILVR KADMADIPGHVFTRGMIIAVLILVAVVCLVTVCVIYRVDLVLFYRHLTRRDETLTDGKTYDA FVSYLKECRPENGEEHTFAVEILPRVLEKHFGYKLCIFERDVVPGGAVVDEIHSLIEKSRRLIIV LSKSYMSNEVRYELESGLHEALVERKIKIILIEFTPVTDFTFLPQSLKLLKSHRVLKWKADKSL SYNSRFWKNLLYLMPAKTVKPGRDEPEVLPVLSES), which is the amino acid sequence of human IL18R1 protein (NCBI Reference Sequence No. NP 003846.1). In some embodiments, the IL18R1 polypeptide comprises a recombinant IL18R1 polypeptide. In some embodiments, the huIL18R1 comprises an amino acid sequence about 99%, 98%, 97%, 96%, 95%, 94%, 93%, 92%, 91%, or 90% homologous to SEQ ID NO: 8. In some embodiments, the recombinant huIL18R1 precursor protein comprises SEQ ID NO: 9 (MNCKKLLLENNKNPTIKKNAEFEDQGYYSCVHFLHHNGKLFNITKTFNITIVEDRSNIVPVLL GPKLNHVAVELGKNVRLNCSALLNEEDVIYWMFGEENGSDPNIHEEKEMRIMTPEGKWHAS KVLRIENIGESNLNVLYNCTVAS TGGTDTKSFILVRKDMADIPGHVFTRGMIIAVLILVAVVCL VTVCVIYRVDLVLFYRHLTRRDETLTDGKTYDAFVSYLKECRPENGEEHTFAVEILPRVLEK HFGYKLCIFERDVVPGGAVVDEIHSLIEKSRRLIIVLSKSYMSNEVRYELESGLHEALVERKIKI ILIEFTPVTDFTFLPQSLKLLKSHRVLKWKADKSLSYNSRFWKNLLYLMPAKTVKPGRDEPEV LPVLSES), which is the amino acid sequence of human IL18R1 (NCBI Reference Sequence No. NP_001269328). In some embodiments, the huIL18R1 comprises an amino acid sequence about 99%, 98%, 97%, 96%, 95%, 94%, 93%, 92%, 91%, or 90% homologous to SEQ ID NO: 9.

In some instances, the IL18R1 polypeptide is truncated. In some instances, the truncation is an N-terminal deletion. In other instances, the truncation is a C-terminal deletion. In additional instances, the truncation comprises both N-terminal and C-terminal deletions. For example, the truncation can be a deletion of at least or about 1, 2, 3, 4, 5, 6, 7, 8, 9, 10, 11, 12, 13, 14, 15, 20, or more residues from either the N-terminus or the C-terminus, or both termini. In some cases, the IL18R1 polypeptide comprises an N-terminal deletion of at least or about 1, 2, 3, 4, 5, 6, 7, 8, 9, 10, 11, 12, 13, 14, 15, 20, or more residues. In some cases, the IL18R1 polypeptide comprises an N-terminal deletion of at least or about 1, 2, 3, 4, 5, 6, 7, 8, 9, or 10 residues. In some cases, the IL18R1 polypeptide comprises an N-terminal deletion of at least or about 2 residues. In some cases, the IL18R1 polypeptide comprises an N-terminal deletion of at least or about 3 residues. In some cases, the IL18R1 polypeptide comprises an N-terminal deletion of at least or about 4 residues. In some cases, the IL18R1 polypeptide comprises an N-terminal deletion of at least or about 5 residues. In some cases, the IL18R1 polypeptide comprises an N-terminal deletion of at least or about 6 residues. In some cases, the IL18R1 polypeptide comprises an N-terminal deletion of at least or about 7 residues. In some cases, the IL18R1 polypeptide comprises an N-terminal deletion of at least or about 8 residues. In some cases, the IL18R1 polypeptide comprises an N-terminal deletion of at least or about 9 residues. In some cases, the IL18R1 polypeptide comprises an N-terminal deletion of at least or about 10 residues.

In some embodiments, the IL18R1 polypeptide has an enhanced plasma half-life. In some instances, the plasma half-life comprises at least 30 minutes, 45 minutes, 60 minutes, 75 minutes, or 90 minutes, 2 hours, 3 hours, 4 hours, 5 hours, 6 hours, 7 hours, 8 hours, 9 hours, 10 hours, 11 hours, 12 hours, 18 hours, 24 hours, 36 hours, 48 hours, 3 days, 4 days, 5 days, 6 days, 7 days, 10 days, 12 days, 14 days, 21 days, 28 days, 30 days, or longer than the plasma half-life of the wild-type IL18R1 protein.

In some embodiments, the IL18R1 polypeptide is a conjugate. In some embodiments, the IL18R1 conjugate comprises an IL18R1 polypeptide comprising at least one amino acid and a conjugating moiety bound to the at least one 1 amino acid. In some embodiments, the at least one amino acid is located proximal to the N-terminus (e.g., proximal to the N-terminal residue). For example, the at least one amino acid is located optionally within the first 10, 20, 30, 40, or 50 residues from the N-terminus. In some cases, the at least one amino acid is located at the N-terminus (i.e., the at least one amino acid is the N-terminal residue of the IL18R1 polypeptide). In other embodiments, the at least one amino acid is located proximal to the C-terminus (e.g., proximal to the C-terminal residue). For example, the at least one amino acid is located optionally within the first 10, 20, 30, 40, or 50 residues from the C-terminus. In some cases, the at least one amino acid is located at the C-terminus (i.e., the at least one amino acid is the C-terminal residue of the IL18R1 polypeptide). In some instances, the IL18R1 conjugate has an enhanced plasma half-life, such as the half-lives described herein. In some embodiments, the IL18R1 conjugate is functionally active (e.g., retains activity). In some embodiments, the IL18R1 conjugate is not functionally active (e.g., devoid of activity). In some embodiments, the conjugating moiety comprises a polymer comprising Polyethylene glycol (PEG).

In some embodiments, the IL18R1 polypeptide is fused with a second polypeptide. In some embodiments, the second polypeptide comprises a polypeptide with a long plasma half-life relative to the plasma half-life of the IL18R1 polypeptide. In some embodiments, the second polypeptide comprises an antibody or antibody fragment. In some embodiments, the antibody or antibody fragment comprises an IgG1, IgG2, IgG4, IgG3, or IgE. In some embodiments, the IgG is an Fc. In some embodiments, the IgG Fc is human. In some instances, the long plasma half-life polypeptide comprises HSA, transferrin, IgA monomer, Retinol-binding protein, Factor H, Factor XIII, C-reactive protein, Factor IX, Fibrinogen, IFN-alpha, Pentameric IgM, IL-2, or Thyroglobulin.

Dosages and Routes of Administration

In general, methods disclosed herein comprise administering a therapeutic agent by oral administration. However, in some instances, methods comprise administering a therapeutic agent by intraperitoneal injection. In some instances, methods comprise administering a therapeutic agent in the form of an anal suppository. In some instances, methods comprise administering a therapeutic agent by intravenous (“i.v.”) administration. It is conceivable that one may also administer therapeutic agents disclosed herein by other routes, such as subcutaneous injection, intramuscular injection, intradermal injection, transdermal injection percutaneous administration, intranasal administration, intralymphatic injection, rectal administration intragastric administration, or any other suitable parenteral administration. In some embodiments, routes for local delivery closer to site of injury or inflammation are preferred over systemic routes. Routes, dosage, time points, and duration of administrating therapeutics may be adjusted. In some embodiments, administration of therapeutics is prior to, or after, onset of either, or both, acute and chronic symptoms of the disease or condition.

An effective dose and dosage of therapeutics to prevent or treat the disease or condition disclosed herein is defined by an observed beneficial response related to the disease or condition, or symptom of the disease or condition. Beneficial response comprises preventing, alleviating, arresting, or curing the disease or condition, or symptom of the disease or condition (e.g., reduced instances of diarrhea, rectal bleeding, weight loss, and size or number of intestinal lesions or strictures, reduced fibrosis or fibrogenesis, reduced fibrostenosis, reduced inflammation). In some embodiments, the beneficial response may be measured by detecting a measurable improvement in the presence, level, or activity, of biomarkers, transcriptomic risk profile, or intestinal microbiome in the subject. An “improvement,” as used herein refers to shift in the presence, level, or activity towards a presence, level, or activity, observed in normal individuals (e.g. individuals who do not suffer from the disease or condition). In instances wherein the therapeutic agent is not therapeutically effective or is not providing a sufficient alleviation of the disease or condition, or symptom of the disease or condition, then the dosage amount and/or route of administration may be changed, or an additional agent may be administered to the subject, along with the therapeutic agent. In some embodiments, as a patient is started on a regimen of a therapeutic agent, the patient is also weaned off (e.g., step-wise decrease in dose) a second treatment regimen.

Suitable dose and dosage administrated to a subject is determined by factors including, but no limited to, the particular therapeutic agent, disease condition and its severity, the identity (e.g., weight, sex, age) of the subject in need of treatment, and can be determined according to the particular circumstances surrounding the case, including, e.g., the specific agent being administered, the route of administration, the condition being treated, and the subject or host being treated. In general, however, doses employed for adult human treatment are typically in the range of 0.01 mg-5000 mg per day. In one aspect, doses employed for adult human treatment are from about 1 mg to about 1000 mg per day. In one embodiment, the desired dose is conveniently presented in a single dose or in divided doses administered simultaneously (or over a short period of time) or at appropriate intervals, for example as two, three, four or more sub-doses per day. Non-limiting examples of effective dosages of for oral delivery of a therapeutic agent include between about 0.1 mg/kg and about 100 mg/kg of body weight per day, and preferably between about 0.5 mg/kg and about 50 mg/kg of body weight per day. In other instances, the oral delivery dosage of effective amount is about 1 mg/kg and about 10 mg/kg of body weight per day of active material. Non-limiting examples of effective dosages for intravenous administration of the therapeutic agent include at a rate between about 0.01 to 100 pmol/kg body weight/min. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the therapeutic agent used, the disease or condition to be treated, the mode of administration, the requirements of the individual subject, the severity of the disease or condition being treated, and the judgment of the practitioner.

In some embodiments, the administration of the therapeutic agent is hourly, once every 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours 22 hours, 23 hours, 1 day, 2 days, 3 days, 4 days, 5 days, 6 days, 7 days, 8 days, 9 days, 10 days, 11 days, 12 days, 13 days, 14 days, 15 days, 1 month, 2 months, 3 months, 4 months, 5 months, 6 months, 7 months, 8 months, 9 months, 10 months, 11 months, 1 year, 2 years, 3 years, 4 years, or 5 years, or 10 years. The effective dosage ranges may be adjusted based on subject's response to the treatment. Some routes of administration will require higher concentrations of effective amount of therapeutics than other routes.

In certain embodiments wherein the patient's condition does not improve, upon the doctor's discretion the administration of therapeutic agent is administered chronically, that is, for an extended period of time, including throughout the duration of the patient's life in order to ameliorate or otherwise control or limit the symptoms of the patient's disease or condition. In certain embodiments wherein a patient's status does improve, the dose of therapeutic agent being administered may be temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. In certain embodiments, the dose of drug being administered may be temporarily reduced or temporarily suspended for a certain length of time (i.e., a “drug diversion”). In specific embodiments, the length of the drug diversion is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug diversion is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. After a suitable length of time, the normal dosing schedule is optionally reinstated.

In some embodiments, once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

Toxicity and therapeutic efficacy of such therapeutic regimens are determined by standard pharmaceutical procedures in cell cultures or experimental animals, including, but not limited to, the determination of the LD50 and the ED50. The dose ratio between the toxic and therapeutic effects is the therapeutic index and it is expressed as the ratio between LD50 and ED50. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the therapeutic agent described herein lies within a range of circulating concentrations that include the ED50 with minimal toxicity. In certain embodiments, the daily dosage range and/or the unit dosage amount varies within this range depending upon the dosage form employed and the route of administration utilized.

Additional Therapeutic

A therapeutic agent may be used alone or in combination with an additional therapeutic agent. In some cases, an “additional therapeutic agent” as used herein is administered alone. The therapeutic agents may be administered together or sequentially. The combination therapies may be administered within the same day, or may be administered one or more days, weeks, months, or years apart. In some cases, a therapeutic agent provided herein is administered if the subject is determined to be non-responsive to a first line of therapy, e.g., such as TNF inhibitor. Such determination may be made by treatment with the first line therapy and monitoring of disease state and/or diagnostic determination that the subject would be non-responsive to the first line therapy.

In some embodiments, the additional therapeutic agent comprises an anti-TNF therapy, e.g., an anti-TNFα therapy. In some embodiments, the additional therapeutic agent comprises a second-line treatment to an anti-TNF therapy. In some embodiments, the additional therapeutic agent comprises an immunosuppressant, or a class of drugs that suppress, or reduce, the strength of the immune system. In some embodiments, the immunosuppressant is an antibody. Non-limiting examples of immunosuppressant therapeutic agents include STELARA® (ustekinumab) azathioprine (AZA), 6-mercaptopurine (6-MP), methotrexate, cyclosporin A. (CsA).

In some embodiments, the additional therapeutic agent comprises a selective anti-inflammatory drug, or a class of drugs that specifically target pro-inflammatory molecules in the body. In some embodiments, the anti-inflammatory drug comprises an antibody. In some embodiments, the anti-inflammatory drug comprises a small molecule. Non-limiting examples of anti-inflammatory drugs include ENTYVIO (vedolizumab), corticosteroids, aminosalicylates, mesalamine, balsalazide (Colazal) and olsalazine (Dipentum).

In some embodiments, the additional therapeutic agent comprises a stem cell therapy. The stem cell therapy may be embryonic or somatic stem cells. The stem cells may be isolated from a donor (allogeneic) or isolated from the subject (autologous). The stem cells may be expanded adipose-derived stem cells (eASCs), hematopoietic stem cells (HSCs), mesenchymal stem (stromal) cells (MSCs), or induced pluripotent stem cells (iPSCs) derived from the cells of the subject. In some embodiments, the therapeutic agent comprises Cx601/Alofisel® (darvadstrocel).

In some embodiments, the additional therapeutic agent comprises a small molecule. The small molecule may be used to treat inflammatory diseases or conditions, or fibrostenonic or fibrotic disease. Non-limiting examples of small molecules include Otezla® (apremilast), alicaforsen, or ozanimod (RPC-1063).

In some embodiments, the additional therapeutic agent comprises an agonist of TL1A, JAK1, GPR35, ADCY7, IFNG, TNFSF8, PFKFB3, SKAP2 GPR65, SPRED2, IL18RAP, GSDMB, and gene expression products from genes implicated in the pathogenesis of inflammatory, fibrotic, or fibrostenotic disease. The therapeutic agent may be an allosteric modulator of TL1A, JAK1, GPR35, ADCY7, IFNG, TNFSF8, PFKFB3, SKAP2 GPR65, SPRED2, IL18RAP, GSDMB, and gene expression products from genes implicated in the pathogenesis of inflammatory, fibrotic, or fibrostenotic disease.

In some embodiments, the additional therapeutic agent comprises an antagonist. The antagonist may comprise an inhibitor of the activity or expression of TL1A, JAK1, GPR35, ADCY7, IFNG, TNFSF8, PFKFB3, SKAP2 GPR65, SPRED2, IL18R1, GSDMB, and gene expression products from genes implicated in the pathogenesis of inflammatory, fibrotic, or fibrostenotic disease. Non-limiting examples of JAK1 inhibitors include Ruxolitinib (INCB018424), S-Ruxolitinib (INCB018424), Baricitinib (LY3009104, INCB028050), Filgotinib (GLPG0634), Momelotinib (CYT387), Cerdulatinib (PRT062070, PRT2070), LY2784544, NVP-BSK805, 2HCl, Tofacitinib (CP-690550, Tasocitinib), XL019, Pacritinib (SB1518), or ZM 39923 HCl.

In some embodiments the additional therapeutic agent comprises an inhibitor of TL1A expression or activity. In some cases, the inhibitor of TL1A expression or activity is effective to inhibit TL1A-DR3 binding. In some embodiments, the inhibitor of TL1A expression or activity comprises an allosteric modulator of TL1A. An allosteric modulator of TL1A may indirectly influence the effects TL1A on DR3, or TR6/DcR3 on TL1A or DR3. The inhibitor of TL1A expression or activity may be a direct inhibitor or indirect inhibitor. Non-limiting examples of an inhibitor of TL1A expression include RNA to protein TL1A translation inhibitors, antisense oligonucleotides targeting the TNFSF15 mRNA (such as miRNAs, or siRNA), epigenetic editing (such as targeting the DNA-binding domain of TNFSF15, or post-translational modifications of histone tails and/or DNA molecules). Non-limiting examples of an inhibitor of TL1A activity include antagonists to the TL1A receptors, (DR3 and TR6/DcR3), antagonists to TL1A antigen, and antagonists to gene expression products involved in TL1A mediated disease. Antagonists as disclosed herein, may include, but are not limited to, an anti-TL1A antibody, an anti-TL1A-binding antibody fragment, or a small molecule. The small molecule may be a small molecule that binds to TL1A or DR3. The anti-TL1A antibody may be monoclonal or polyclonal. The anti-TL1A antibody may be humanized or chimeric. The anti-TL1A antibody may be a fusion protein. The anti-TL1A antibody may be a blocking anti-TL1A antibody. A blocking antibody blocks binding between two proteins, e.g., a ligand and its receptor. Therefore, a TL1A blocking antibody includes an antibody that prevents binding of TL1A to DR3 or TR6/DcR3 receptors. In anon-limiting example, the TL1A blocking antibody binds to DR3. In another example, the TL1A blocking antibody binds to DcR3. In some cases, the TL1A antibody is an anti-TL1A antibody that specifically binds to TL1A.

In some embodiments the additional therapeutic agent comprises an inhibitor of CD30L expression or activity. The inhibitor of CD30L expression or activity may be a direct inhibitor or indirect inhibitor. Non-limiting examples of an inhibitor of CD30L expression include RNA to protein TL1A translation inhibitors, antisense oligonucleotides targeting the mRNA (such as miRNAs, or siRNA), epigenetic editing (such as targeting the DNA-binding domain of CD30L, or post-translational modifications of histone tails and/or DNA molecules). In some embodiments, the CD30L inhibitor is an anti-CD30L antibody. The anti-CD30L antibody may be monoclonal or polyclonal. The anti-CD30L antibody may be humanized or chimeric.

In some instances, the additional therapeutic agent comprises administering to the subject an active agent that modulates CARD9 activity or expression. In various embodiments, the inhibitor of CARD9 activity or expression comprises a CARD9 antibody, a small molecule, a direct inhibitor of CARD9, an indirect inhibitor of CARD9, an allosteric modulator of CARD9, an anti-CARD9 antibody or antibody fragment, antibody or antibody fragment that specifically binds to Rubicon, an anti-ripartite Motif Containing 62 (TRIM62) antibody or antibody fragment, an antibody or antibody fragment that specifically binds to B Cell CLL/Lymphoma 10 (BCL10), an inhibitor of CARD9-Rubicon interaction, an inhibitor of CARD9-Tripartite Motif Containing 62 (TRIM62) interaction, an inhibitor of CARD9-B Cell CLL/Lymphoma 10 (BCL10) interaction, a small molecule that specifically binds CARD9 a small molecule that specifically binds to Rubicon, a small molecule that specifically binds to Tripartite Motif Containing 62 (TRIM62), a small molecule that specifically binds to B Cell CLL/Lymphoma 10 (BCL10), an inhibitor of CARD9-Rubicon interaction, an inhibitor of CARD9-Tripartite Motif Containing 62 (TRIM62) interaction, an inhibitor of B Cell CLL/Lymphoma 10 (BCL10)-CARD9 interaction, or a combination thereof. In some other embodiments, the inhibitor of CARD9 activity or expression comprises the small molecule inhibitor BRD5529, BRD4203, BRD8991, BRD4098 or a combination thereof. In some embodiments, the CARD9 antibody recognizes the total CARD9 protein. In other embodiments, the CARD9 antibody recognizes 90%, 80%, 70%, 60%, 50%, 40%, 30%, 20%, or 10% of the total CARD9 protein. In some embodiments, the modulator of CARD9 comprises a stem cell therapy. The stem cell therapy may be embryonic or somatic stem cells. The stem cells may be isolated from a donor (allogeneic) or isolated from the subject (autologous). The stem cells may be expanded adipose-derived stem cells (eASCs), hematopoietic stem cells (HSCs), mesenchymal stem (stromal) cells (MSCs), or induced pluripotent stem cells (iPSCs) derived from the cells of the subject.

In some embodiments, the additional therapeutic agent comprises administering to the subject an antibody or antibody fragment, a small molecule, an allosteric modulator, an agonist, an antagonist, a direct modulator of Dectin-1A, an indirect modulator of Dectin-1A, or a combination thereof. In other embodiments, the treatment is an inhibitor of C-type lectin-like receptors. In various embodiments, the agonist is soluble β-glucan antagonist laminarin. In various other embodiments, the antagonist is soluble β-glucan antagonist laminarin. In some embodiments, the antibody binds to the C-type lectin-like receptors. In some embodiments, the Dectin-1 antibody recognizes the total Dectin-1 protein. In other embodiments, the Dectin-1 antibody recognizes 90%, 80%, 70%, 60%, 50%, 40%, 30%, 20%, or 10% of the total Dectine-1A protein. In some embodiments, the modulator of Dectin-1A comprises a stem cell therapy. The stem cell therapy may be embryonic or somatic stem cells. The stem cells may be isolated from a donor (allogeneic) or isolated from the subject (autologous). The stem cells may be expanded adipose-derived stem cells (eASCs), hematopoietic stem cells (HSCs), mesenchymal stem (stromal) cells (MSCs), or induced pluripotent stem cells (iPSCs) derived from the cells of the subject.

In some instances, the additional therapeutic agent comprises administering to the subject an antimycotic agent. In some instances, the antimycotic agent comprises an active agent that inhibits growth of a fungus. In some instances, the antimycotic agent comprises an active agent that kills a fungus. In some embodiments, the antimycotic agent comprises polyene, an azole, an echinocandin, an flucytosine, an allylamine, a tolnaftate, or griseofulvin, or a combination thereof. In other embodiments, the azole comprises triazole, imidazole, clotrimazole, ketoconazole, itraconazole, terconazole, oxiconazole, miconazole, econazole, tioconazole, voriconazole, fluconazole, isavuconazole, itraconazole, pramiconazole, ravuconazole, or posaconazole. In some other embodiments, the polyene comprises amphotericin B, nystatin, or natamycin. In yet other embodiments, the echinocandin comprises caspofungin, anidulafungin, or micafungin. In various other embodiments, the allylamine comprises naftifine or terbinafine.

Pharmaceutical Composition

A pharmaceutical composition, as used herein, refers to a mixture of a therapeutic agent, with other chemical components (i.e. pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. Optionally, the compositions include two or more therapeutic agent (e.g., one or more therapeutic agents and one or more additional agents) as discussed herein. In practicing the methods of treatment or use provided herein, therapeutically effective amounts of therapeutic agents described herein are administered in a pharmaceutical composition to a mammal having a disease, disorder, or condition to be treated, e.g., an inflammatory disease, fibrostenotic disease, and/or fibrotic disease. In some embodiments, the mammal is a human. A therapeutically effective amount can vary widely depending on the severity of the disease, the age and relative health of the subject, the potency of the therapeutic agent used and other factors. The therapeutic agents can be used singly or in combination with one or more therapeutic agents as components of mixtures.

The pharmaceutical formulations described herein are administered to a subject by appropriate administration routes, including but not limited to, intravenous, intraarterial, oral, parenteral, buccal, topical, transdermal, rectal, intramuscular, subcutaneous, intraosseous, transmucosal, inhalation, or intraperitoneal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Pharmaceutical compositions including a therapeutic agent are manufactured in a conventional manner, such as, by way of example only, by means of conventional mixing, dissolving, granulating, dragee-making, levigating, emulsifying, encapsulating, entrapping or compression processes.

The pharmaceutical compositions may include at least a therapeutic agent as an active ingredient in free-acid or free-base form, or in a pharmaceutically acceptable salt form. In addition, the methods and pharmaceutical compositions described herein include the use of N-oxides (if appropriate), crystalline forms, amorphous phases, as well as active metabolites of these compounds having the same type of activity. In some embodiments, therapeutic agents exist in unsolvated form or in solvated forms with pharmaceutically acceptable solvents such as water, ethanol, and the like. The solvated forms of the therapeutic agents are also considered to be disclosed herein.

In some embodiments, a therapeutic agent exists as a tautomer. All tautomers are included within the scope of the agents presented herein. As such, it is to be understood that a therapeutic agent or a salt thereof may exhibit the phenomenon of tautomerism whereby two chemical compounds that are capable of facile interconversion by exchanging a hydrogen atom between two atoms, to either of which it forms a covalent bond. Since the tautomeric compounds exist in mobile equilibrium with each other they may be regarded as different isomeric forms of the same compound.

In some embodiments, a therapeutic agent exists as an enantiomer, diastereomer, or other steroisomeric form. The agents disclosed herein include all enantiomeric, diastereomeric, and epimeric forms as well as mixtures thereof.

In some embodiments, therapeutic agents described herein may be prepared as prodrugs. A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a therapeutic agent described herein, which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the therapeutic agent. In certain embodiments, a prodrug is enzymatically metabolized by one or more steps or processes to the biologically, pharmaceutically or therapeutically active form of the therapeutic agent.

Prodrug forms of the therapeutic agents, wherein the prodrug is metabolized in vivo to produce an agent as set forth herein are included within the scope of the claims. Prodrug forms of the herein described therapeutic agents, wherein the prodrug is metabolized in vivo to produce an agent as set forth herein are included within the scope of the claims. In some cases, some of the therapeutic agents described herein may be a prodrug for another derivative or active compound. In some embodiments described herein, hydrazones are metabolized in vivo to produce a therapeutic agent.

In certain embodiments, compositions provided herein include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In some embodiments, formulations described herein benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

The pharmaceutical compositions described herein are formulated into any suitable dosage form, including but not limited to, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In one aspect, a therapeutic agent as discussed herein, e.g., therapeutic agent is formulated into a pharmaceutical composition suitable for intramuscular, subcutaneous, or intravenous injection. In one aspect, formulations suitable for intramuscular, subcutaneous, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles include water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. In some embodiments, formulations suitable for subcutaneous injection also contain additives such as preserving, wetting, emulsifying, and dispensing agents. Prevention of the growth of microorganisms can be ensured by various antibacterial and antifungal agents, such as parabens, chlorobutanol, phenol, sorbic acid, and the like. In some cases it is desirable to include isotonic agents, such as sugars, sodium chloride, and the like. Prolonged absorption of the injectable pharmaceutical form can be brought about by the use of agents delaying absorption, such as aluminum monostearate and gelatin.

For intravenous injections or drips or infusions, a therapeutic agent described herein is formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations include aqueous or nonaqueous solutions, preferably with physiologically compatible buffers or excipients. Such excipients are known.

Parenteral injections may involve bolus injection or continuous infusion. Formulations for injection may be presented in unit dosage form, e.g., in ampoules or in multi-dose containers, with an added preservative. The pharmaceutical composition described herein may be in a form suitable for parenteral injection as a sterile suspensions, solutions or emulsions in oily or aqueous vehicles, and may contain formulatory agents such as suspending, stabilizing and/or dispersing agents. In one aspect, the active ingredient is in powder form for constitution with a suitable vehicle, e.g., sterile pyrogen-free water, before use.

For administration by inhalation, a therapeutic agent is formulated for use as an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the therapeutic agent described herein and a suitable powder base such as lactose or starch.

Representative intranasal formulations are described in, for example, U.S. Pat. Nos. 4,476,116, 5,116,817 and 6,391,452. Formulations that include a therapeutic agent are prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are known to those skilled in the preparation of nasal dosage forms and some of these can be found in REMINGTON: THE SCIENCE AND PRACTICE OF PHARMACY, 21st edition, 2005. The choice of suitable carriers is dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present. Preferably, the nasal dosage form should be isotonic with nasal secretions.

Pharmaceutical preparations for oral use are obtained by mixing one or more solid excipient with one or more of the therapeutic agents described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are added, such as the cross-linked croscarmellose sodium, polyvinylpyrrolidone, agar, or alginic acid or a salt thereof such as sodium alginate. In some embodiments, dyestuffs or pigments are added to the tablets or dragee coatings for identification or to characterize different combinations of active therapeutic agent doses.

In some embodiments, pharmaceutical formulations of a therapeutic agent are in the form of a capsules, including push-fit capsules made of gelatin, as well as soft, sealed capsules made of gelatin and a plasticizer, such as glycerol or sorbitol. The push-fit capsules contain the active ingredients in admixture with filler such as lactose, binders such as starches, and/or lubricants such as talc or magnesium stearate and, optionally, stabilizers. In soft capsules, the active therapeutic agent is dissolved or suspended in suitable liquids, such as fatty oils, liquid paraffin, or liquid polyethylene glycols. In some embodiments, stabilizers are added. A capsule may be prepared, for example, by placing the bulk blend of the formulation of the therapeutic agent inside of a capsule. In some embodiments, the formulations (non-aqueous suspensions and solutions) are placed in a soft gelatin capsule. In other embodiments, the formulations are placed in standard gelatin capsules or non-gelatin capsules such as capsules comprising HPMC. In other embodiments, the formulation is placed in a sprinkle capsule, wherein the capsule is swallowed whole or the capsule is opened and the contents sprinkled on food prior to eating.

All formulations for oral administration are in dosages suitable for such administration. In one aspect, solid oral dosage forms are prepared by mixing a therapeutic agent with one or more of the following: antioxidants, flavoring agents, and carrier materials such as binders, suspending agents, disintegration agents, filling agents, surfactants, solubilizers, stabilizers, lubricants, wetting agents, and diluents. In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder, a capsule, solid dispersion, solid solution, bioerodible dosage form, controlled release formulations, pulsatile release dosage forms, multiparticulate dosage forms, beads, pellets, granules. In other embodiments, the pharmaceutical formulation is in the form of a powder. Compressed tablets are solid dosage forms prepared by compacting the bulk blend of the formulations described above. In various embodiments, tablets will include one or more flavoring agents. In other embodiments, the tablets will include a film surrounding the final compressed tablet. In some embodiments, the film coating can provide a delayed release of a therapeutic agent from the formulation. In other embodiments, the film coating aids in patient compliance (e.g., Opadry® coatings or sugar coating). Film coatings including Opadry® typically range from about 1% to about 3% of the tablet weight. In some embodiments, solid dosage forms, e.g., tablets, effervescent tablets, and capsules, are prepared by mixing particles of a therapeutic agent with one or more pharmaceutical excipients to form a bulk blend composition. The bulk blend is readily subdivided into equally effective unit dosage forms, such as tablets, pills, and capsules. In some embodiments, the individual unit dosages include film coatings. These formulations are manufactured by conventional formulation techniques.

In another aspect, dosage forms include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents. Exemplary useful microencapsulation materials include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG, HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, Eudragit® NE30D, and Eudragit® NE 40D, cellulose acetate phthalate, sepifilms such as mixtures of HPMC and stearic acid, cyclodextrins, and mixtures of these materials.

Liquid formulation dosage forms for oral administration are optionally aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to therapeutic agent the liquid dosage forms optionally include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further includes a crystal-forming inhibitor.

In some embodiments, the pharmaceutical formulations described herein are self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase is optionally added just prior to administration, which ensures stability of an unstable or hydrophobic active ingredient. Thus, the SEDDS provides an effective delivery system for oral and parenteral delivery of hydrophobic active ingredients. In some embodiments, SEDDS provides improvements in the bioavailability of hydrophobic active ingredients. Methods of producing self-emulsifying dosage forms include, but are not limited to, for example, U.S. Pat. Nos. 5,858,401, 6,667,048, and 6,960,563.

Buccal formulations that include a therapeutic agent are administered using a variety of formulations known in the art. For example, such formulations include, but are not limited to, U.S. Pat. Nos. 4,229,447, 4,596,795, 4,755,386, and 5,739,136. In addition, the buccal dosage forms described herein can further include a bioerodible (hydrolysable) polymeric carrier that also serves to adhere the dosage form to the buccal mucosa. For buccal or sublingual administration, the compositions may take the form of tablets, lozenges, or gels formulated in a conventional manner.

For intravenous injections, a therapeutic agent is optionally formulated in aqueous solutions, preferably in physiologically compatible buffers such as Hank's solution, Ringer's solution, or physiological saline buffer. For transmucosal administration, penetrants appropriate to the barrier to be permeated are used in the formulation. For other parenteral injections, appropriate formulations include aqueous or nonaqueous solutions, preferably with physiologically compatible buffers or excipients.

Parenteral injections optionally involve bolus injection or continuous infusion. Formulations for injection are optionally presented in unit dosage form, e.g., in ampoules or in multi dose containers, with an added preservative. In some embodiments, a pharmaceutical composition described herein is in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of an agent that modulates the activity of a carotid body in water soluble form. Additionally, suspensions of an agent that modulates the activity of a carotid body are optionally prepared as appropriate, e.g., oily injection suspensions.

Conventional formulation techniques include, e.g., one or a combination of methods: (1) dry mixing, (2) direct compression, (3) milling, (4) dry or non-aqueous granulation, (5) wet granulation, or (6) fusion. Other methods include, e.g., spray drying, pan coating, melt granulation, granulation, fluidized bed spray drying or coating (e.g., wurster coating), tangential coating, top spraying, tableting, extruding and the like.

Suitable carriers for use in the solid dosage forms described herein include, but are not limited to, acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include, but are not limited to, lactose, calcium carbonate, calcium phosphate, dibasic calcium phosphate, calcium sulfate, microcrystalline cellulose, cellulose powder, dextrose, dextrates, dextran, starches, pregelatinized starch, hydroxypropylmethycellulose (HPMC), hydroxypropylmethycellulose phthalate, hydroxypropylmethylcellulose acetate stearate (HPMCAS), sucrose, xylitol, lactitol, mannitol, sorbitol, sodium chloride, polyethylene glycol, and the like.

Suitable disintegrants for use in the solid dosage forms described herein include, but are not limited to, natural starch such as corn starch or potato starch, a pregelatinized starch, or sodium starch glycolate, a cellulose such as methylcrystalline cellulose, methylcellulose, microcrystalline cellulose, croscarmellose, or a cross-linked cellulose, such as cross-linked sodium carboxymethylcellulose, cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a gum such as agar, guar, locust bean, Karaya, pectin, or tragacanth, sodium starch glycolate, bentonite, sodium lauryl sulfate, sodium lauryl sulfate in combination starch, and the like.

Binders impart cohesiveness to solid oral dosage form formulations: for powder filled capsule formulation, they aid in plug formation that can be filled into soft or hard shell capsules and for tablet formulation, they ensure the tablet remaining intact after compression and help assure blend uniformity prior to a compression or fill step. Materials suitable for use as binders in the solid dosage forms described herein include, but are not limited to, carboxymethylcellulose, methylcellulose, hydroxypropylmethylcellulose, hydroxypropylmethylcellulose acetate stearate, hydroxyethylcellulose, hydroxypropylcellulose, ethylcellulose, and microcrystalline cellulose, microcrystalline dextrose, amylose, magnesium aluminum silicate, polysaccharide acids, bentonites, gelatin, polyvinylpyrrolidone/vinyl acetate copolymer, crospovidone, povidone, starch, pregelatinized starch, tragacanth, dextrin, a sugar, such as sucrose, glucose, dextrose, molasses, mannitol, sorbitol, xylitol, lactose, a natural or synthetic gum such as acacia, tragacanth, ghatti gum, mucilage of isapol husks, starch, polyvinylpyrrolidone, larch arabogalactan, polyethylene glycol, waxes, sodium alginate, and the like.

In general, binder levels of 20-70% are used in powder-filled gelatin capsule formulations. Binder usage level in tablet formulations varies whether direct compression, wet granulation, roller compaction, or usage of other excipients such as fillers which itself can act as moderate binder. Binder levels of up to 70% in tablet formulations is common.

Suitable lubricants or glidants for use in the solid dosage forms described herein include, but are not limited to, stearic acid, calcium hydroxide, talc, corn starch, sodium stearyl fumerate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include, but are not limited to, sugars (including lactose, sucrose, and dextrose), polysaccharides (including dextrates and maltodextrin), polyols (including mannitol, xylitol, and sorbitol), cyclodextrins and the like.

Suitable wetting agents for use in the solid dosage forms described herein include, for example, oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, vitamin E TPGS and the like.

Suitable surfactants for use in the solid dosage forms described herein include, for example, sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like.

Suitable suspending agents for use in the solid dosage forms described here include, but are not limited to, polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 5400, vinyl pyrrolidone/vinyl acetate copolymer (S630), sodium carboxymethylcellulose, methylcellulose, hydroxy-propylmethylcellulose, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Suitable antioxidants for use in the solid dosage forms described herein include, for example, e.g., butylated hydroxytoluene (BHT), sodium ascorbate, and tocopherol.

It should be appreciated that there is considerable overlap between additives used in the solid dosage forms described herein. Thus, the above-listed additives should be taken as merely exemplary, and not limiting, of the types of additives that can be included in solid dosage forms of the pharmaceutical compositions described herein. The amounts of such additives can be readily determined by one skilled in the art, according to the particular properties desired.

In various embodiments, the particles of a therapeutic agents and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the formulation into the gastrointestinal fluid.

In other embodiments, a powder including a therapeutic agent is formulated to include one or more pharmaceutical excipients and flavors. Such a powder is prepared, for example, by mixing the therapeutic agent and optional pharmaceutical excipients to form a bulk blend composition. Additional embodiments also include a suspending agent and/or a wetting agent. This bulk blend is uniformly subdivided into unit dosage packaging or multi-dosage packaging units.

In still other embodiments, effervescent powders are also prepared. Effervescent salts have been used to disperse medicines in water for oral administration.

In some embodiments, the pharmaceutical dosage forms are formulated to provide a controlled release of a therapeutic agent. Controlled release refers to the release of the therapeutic agent from a dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a subject over an extended period of time according to a predetermined profile. Such release rates can provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

In some embodiments, the solid dosage forms described herein are formulated as enteric coated delayed release oral dosage forms, i.e., as an oral dosage form of a pharmaceutical composition as described herein which utilizes an enteric coating to affect release in the small intestine or large intestine. In one aspect, the enteric coated dosage form is a compressed or molded or extruded tablet/mold (coated or uncoated) containing granules, powder, pellets, beads or particles of the active ingredient and/or other composition components, which are themselves coated or uncoated. In one aspect, the enteric coated oral dosage form is in the form of a capsule containing pellets, beads or granules, which include a therapeutic agent that are coated or uncoated.

Any coatings should be applied to a sufficient thickness such that the entire coating does not dissolve in the gastrointestinal fluids at pH below about 5, but does dissolve at pH about 5 and above. Coatings are typically selected from any of the following: Shellac—this coating dissolves in media of pH >7; Acrylic polymers—examples of suitable acrylic polymers include methacrylic acid copolymers and ammonium methacrylate copolymers. The Eudragit series E, L, S, RL, RS and NE (Rohm Pharma) are available as solubilized in organic solvent, aqueous dispersion, or dry powders. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in stomach and dissolve in the intestine; Poly Vinyl Acetate Phthalate (PVAP)-PVAP dissolves in pH >5, and it is much less permeable to water vapor and gastric fluids. Conventional coating techniques such as spray or pan coating are employed to apply coatings. The coating thickness must be sufficient to ensure that the oral dosage form remains intact until the desired site of topical delivery in the intestinal tract is reached.

In other embodiments, the formulations described herein are delivered using a pulsatile dosage form. A pulsatile dosage form is capable of providing one or more immediate release pulses at predetermined time points after a controlled lag time or at specific sites. Exemplary pulsatile dosage forms and methods of their manufacture are disclosed in U.S. Pat. Nos. 5,011,692, 5,017,381, 5,229,135, 5,840,329 and 5,837,284. In one embodiment, the pulsatile dosage form includes at least two groups of particles, (i.e. multiparticulate) each containing the formulation described herein. The first group of particles provides a substantially immediate dose of a therapeutic agent upon ingestion by a mammal. The first group of particles can be either uncoated or include a coating and/or sealant. In one aspect, the second group of particles comprises coated particles. The coating on the second group of particles provides a delay of from about 2 hours to about 7 hours following ingestion before release of the second dose. Suitable coatings for pharmaceutical compositions are described herein or known in the art.

In some embodiments, pharmaceutical formulations are provided that include particles of a therapeutic agent and at least one dispersing agent or suspending agent for oral administration to a subject. The formulations may be a powder and/or granules for suspension, and upon admixture with water, a substantially uniform suspension is obtained.

In some embodiments, particles formulated for controlled release are incorporated in a gel or a patch or a wound dressing.

In one aspect, liquid formulation dosage forms for oral administration and/or for topical administration as a wash are in the form of aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to the particles of a therapeutic agent, the liquid dosage forms include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions can further include a crystalline inhibitor.

In some embodiments, the liquid formulations also include inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, sodium lauryl sulfate, sodium doccusate, cholesterol, cholesterol esters, taurocholic acid, phosphotidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like.

Furthermore, pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, pharmaceutical compositions optionally include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In one embodiment, the aqueous suspensions and dispersions described herein remain in a homogenous state, as defined in The USP Pharmacists' Pharmacopeia (2005 edition, chapter 905), for at least 4 hours. In one embodiment, an aqueous suspension is re-suspended into a homogenous suspension by physical agitation lasting less than 1 minute. In still another embodiment, no agitation is necessary to maintain a homogeneous aqueous dispersion.

Examples of disintegrating agents for use in the aqueous suspensions and dispersions include, but are not limited to, a starch, e.g., a natural starch such as corn starch or potato starch, a pregelatinized starch, or sodium starch glycolate; a cellulose such as methylcrystalline cellulose, methylcellulose, croscarmellose, or a cross-linked cellulose, such as cross-linked sodium carboxymethylcellulose, cross-linked carboxymethylcellulose, or cross-linked croscarmellose; a cross-linked starch such as sodium starch glycolate; a cross-linked polymer such as crospovidone; a cross-linked polyvinylpyrrolidone; alginate such as alginic acid or a salt of alginic acid such as sodium alginate; a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein include, for example, hydrophilic polymers, electrolytes, Tween® 60 or 80, PEG, polyvinylpyrrolidone, and the carbohydrate-based dispersing agents such as, for example, hydroxypropylcellulose and hydroxypropyl cellulose ethers, hydroxypropyl methylcellulose and hydroxypropyl methylcellulose ethers, carboxymethylcellulose sodium, methylcellulose, hydroxyethylcellulose, hydroxypropylmethyl-cellulose phthalate, hydroxypropylmethyl-cellulose acetate stearate, noncrystalline cellulose, magnesium aluminum silicate, triethanolamine, polyvinyl alcohol (PVA), polyvinylpyrrolidone/vinyl acetate copolymer, 4-(1,1,3,3-tetramethylbutyl)-phenol polymer with ethylene oxide and formaldehyde (also known as tyloxapol), poloxamers; and poloxamines. In other embodiments, the dispersing agent is selected from a group not comprising one of the following agents: hydrophilic polymers; electrolytes; Tween® 60 or 80; PEG; polyvinylpyrrolidone (PVP); hydroxypropylcellulose and hydroxypropyl cellulose ethers; hydroxypropyl methylcellulose and hydroxypropyl methylcellulose ethers; carboxymethylcellulose sodium; methylcellulose; hydroxyethylcellulose; hydroxypropylmethyl-cellulose phthalate; hydroxypropylmethyl-cellulose acetate stearate; non-crystalline cellulose; magnesium aluminum silicate; triethanolamine; polyvinyl alcohol (PVA); 4-(1,1,3,3-tetramethylbutyl)-phenol polymer with ethylene oxide and formaldehyde; poloxamers; or poloxamines.

Wetting agents suitable for the aqueous suspensions and dispersions described herein include, but are not limited to, cetyl alcohol, glycerol monostearate, polyoxyethylene sorbitan fatty acid esters (e.g., the commercially available Tweens® such as e.g., Tween 20® and Tween 80®, and polyethylene glycols, oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphotidylcholine and the like.

Suitable preservatives for the aqueous suspensions or dispersions described herein include, for example, potassium sorbate, parabens (e.g., methylparaben and propylparaben), benzoic acid and its salts, other esters of parahydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

Suitable viscosity enhancing agents for the aqueous suspensions or dispersions described herein include, but are not limited to, methyl cellulose, xanthan gum, carboxymethyl cellulose, hydroxypropyl cellulose, hydroxypropylmethyl cellulose, Plasdon® S-630, carbomer, polyvinyl alcohol, alginates, acacia, chitosans and combinations thereof. The concentration of the viscosity enhancing agent will depend upon the agent selected and the viscosity desired.

Examples of sweetening agents suitable for the aqueous suspensions or dispersions described herein include, for example, acacia syrup, acesulfame K, alitame, aspartame, chocolate, cinnamon, citrus, cocoa, cyclamate, dextrose, fructose, ginger, glycyrrhetinate, Glycyrrhiza (licorice) syrup, monoammonium glyrrhizinate (MagnaSweet®), malitol, mannitol, menthol, neohesperidine DC, neotame, Prosweet® Powder, saccharin, sorbitol, stevia, sucralose, sucrose, sodium saccharin, saccharin, aspartame, acesulfame potassium, mannitol, sucralose, tagatose, thaumatin, vanilla, xylitol, or any combination thereof.

In some embodiments, a therapeutic agent is prepared as transdermal dosage form. In some embodiments, the transdermal formulations described herein include at least three components: (1) a therapeutic agent; (2) a penetration enhancer; and (3) an optional aqueous adjuvant. In some embodiments the transdermal formulations include additional components such as, but not limited to, gelling agents, creams and ointment bases, and the like. In some embodiments, the transdermal formulation is presented as a patch or a wound dressing. In some embodiments, the transdermal formulation further include a woven or non-woven backing material to enhance absorption and prevent the removal of the transdermal formulation from the skin. In other embodiments, the transdermal formulations described herein can maintain a saturated or supersaturated state to promote diffusion into the skin.

In one aspect, formulations suitable for transdermal administration of a therapeutic agent described herein employ transdermal delivery devices and transdermal delivery patches and can be lipophilic emulsions or buffered, aqueous solutions, dissolved and/or dispersed in a polymer or an adhesive. In one aspect, such patches are constructed for continuous, pulsatile, or on demand delivery of pharmaceutical agents. Still further, transdermal delivery of the therapeutic agents described herein can be accomplished by means of iontophoretic patches and the like. In one aspect, transdermal patches provide controlled delivery of a therapeutic agent. In one aspect, transdermal devices are in the form of a bandage comprising a backing member, a reservoir containing the therapeutic agent optionally with carriers, optionally a rate controlling barrier to deliver the therapeutic agent to the skin of the host at a controlled and predetermined rate over a prolonged period of time, and means to secure the device to the skin.

In further embodiments, topical formulations include gel formulations (e.g., gel patches which adhere to the skin). In some of such embodiments, a gel composition includes any polymer that forms a gel upon contact with the body (e.g., gel formulations comprising hyaluronic acid, pluronic polymers, poly(lactic-co-glycolic acid (PLGA)-based polymers or the like). In some forms of the compositions, the formulation comprises a low-melting wax such as, but not limited to, a mixture of fatty acid glycerides, optionally in combination with cocoa butter which is first melted. Optionally, the formulations further comprise a moisturizing agent.

In certain embodiments, delivery systems for pharmaceutical therapeutic agents may be employed, such as, for example, liposomes and emulsions. In certain embodiments, compositions provided herein can also include an mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, a therapeutic agent described herein may be administered topically and can be formulated into a variety of topically administrable compositions, such as solutions, suspensions, lotions, gels, pastes, medicated sticks, balms, creams or ointments. Such pharmaceutical therapeutic agents can contain solubilizers, stabilizers, tonicity enhancing agents, buffers and preservatives.

### Kits and Compositions

Compositions

Disclosed herein, in some embodiments, are compositions useful for the detection of a genotype or biomarker in a sample obtained from a subject according to the methods described herein. Aspects disclosed herein provide compositions comprises a polynucleotide sequence comprising at least 10 but less than 50 contiguous nucleotides encoding one or more IL18R1 risk genotypes comprising rs13001325, rs1420101, rs12479210, rs950880, rs13020553, rs13019081, rs12712141, rs2287037, rs1420102, rs12466380, rs1997467, rs1558619, rs1420088, rs12999364, rs4142132, rs12987977, rs11690443, rs1362350, rs12996505, rs873022, rs974389, rs3771177, rs3732129, rs17026974, rs6706844, rs13020793, rs11685480, rs1558622, rs10183388, rs12712135, rs10189711, rs11685424, rs10189202, rs10191914, rs11123918, rs1968171, rs6733174, rs59247511, rs1558620, rs1921622, rs12998521, rs13017455, rs1362349, rs11123923, rs10190555, rs1035127, rs17027087, rs2080289, rs4851570, rs17027060, rs12712145, rs1420098, rs3732123, rs2287034, rs3860444, rs3821203, rs56258475, rs2270298, rs4851006, rs6710885, rs1568681, rs2241117, rs17027037, rs2270297, rs6753717, rs3755274, rs17027071, rs6750020, rs17027006, rs11683700, rs2058622, rs4851007, rs3732126, rs1807782, rs12469506, rs4851575, rs3771172, rs11465633, rs1135354, rs1558627, rs55927292, rs3771171, rs13015714, rs2160202, rs55883125, rs2041740, rs1035130, rs1420103, rs67723747, rs6543116, rs55664618, rs4851005, rs17027056, rs1420089, rs62152661, rs1420095, rs56030066, rs62152714, rs17696376, rs12105808, rs78248680, rs56151044, rs62152662, rs17651485, rs3771170, rs11123926, rs76721133, rs4988955, rs9807962, rs9808453, rs13424006, rs11695627, rs3771166, rs10173193, rs11465575, rs4851566, rs9308857, rs1974675, rs6751967, rs3771162, rs56386507, rs1997466, rs12712140, and/or rs1362348, or reverse complements thereof, wherein the contiguous polynucleotide sequence comprises a detectable molecule. In various embodiments, the detectable molecule comprises a fluorophore. In other embodiments, the polynucleotide sequences further comprise a quencher.

Also disclosed herein are compositions comprising an antibody or antigen-binding fragment that specifically binds to IL18R1, wherein the antibody or antigen-binding fragment comprises a detectable molecule. In various embodiments, the antibody comprises a monoclonal antibody, a chimeric antibody, a CDR-grafted antibody, a Fab, a Fab′, a F(ab′)2, a Fv, a disulfide linked Fv, a scFv, a single domain antibody, a diabody, a multispecific antibody, a dual specific antibody, an anti-idiotypic antibody, or a bispecific antibody. In some embodiments, the antibody or antigen-binding fragment comprises an IgG antibody, an IgM antibody, and/or an IgE antibody. In some embodiments, the detectable molecule comprises a fluorophore. In some embodiments, the antibody or antigen-binding fragment is conjugated to a paramagnetic particle (e.g., bead).

Kits

Disclosed herein, in some embodiments, are kits useful for to detect the genotypes and/or biomarkers disclosed herein. In some embodiments, the kits disclosed herein may be used to diagnose and/or treat a disease or condition in a subject; or select a patient for treatment and/or monitor a treatment disclosed herein. In some embodiments, the kit comprises the compositions described herein, which can be used to perform the methods described herein. Kits comprise an assemblage of materials or components, including at least one of the compositions. Thus, in some embodiments the kit contains a composition including of the pharmaceutical composition, for the treatment of IBD. In other embodiments, the kits contains all of the components necessary and/or sufficient to perform an assay for detecting and measuring IBD markers, including all controls, directions for performing assays, and any necessary software for analysis and presentation of results.

In some instances, the kits described herein comprise components comprising the compositions described herein for detecting the presence, absence, and/or quantity of a target nucleic acid (e.g., IL18R1, IL18R1 SNPs) and/or protein (e.g., IL18R1) described herein. In some embodiments, the kit further comprises components for detecting the presence, absence, and/or quantity of a serological marker described herein. In some embodiments, the kit comprises the compositions (e.g., primers, probes, antibodies) described herein. The disclosure provides kits suitable for assays such as enzyme-linked immunosorbent assay (ELISA), single-molecular array (Simoa), PCR, and qPCR. The exact nature of the components configured in the kit depends on its intended purpose. For example, some embodiments are configured for the purpose of treating a disease or condition disclosed herein (e.g., IBD, CD, UC) in a subject. In some embodiments, the kit is configured particularly for the purpose of treating mammalian subjects. In some embodiments, the kit is configured particularly for the purpose of treating human subjects. In further embodiments, the kit is configured for veterinary applications, treating subjects such as, but not limited to, farm animals, domestic animals, and laboratory animals. In some embodiments, the kit is configured to select a subject for a therapeutic agent, such as those disclosed herein. In some embodiments, the kit is configured to select a subject for treatment with Crohn's disease, inflammatory bowel disease, or ulcerative colitis.

In some embodiments, the kit is used to detect a IL18R1 risk genotype in a sample obtained from a subject in need thereof. In some instances, the IL18R1 risk genotype comprises one or more SNPs and/or indels selected at rs1921622, rs2287037, rs1974675, rs2041739, rs76362690, rs2287037, or rs80256362, or a SNP in linkage disequilibrium therewith. In some instances, the SNP comprises a risk allele which may be a minor allele, a major allele, or an insertion/deletion of a nucleobase. In some instances, the SNP at rs1921622 comprises an “A” or a “G” on a forward DNA strand encoding the SNP. In some instances, the SNP at rs2287037 comprises an “A” or a “G” on a reverse DNA strand encoding the SNP. In some instances, the SNP at rs1974675 comprises a “C” or a “T” on a reverse DNA strand encoding the SNP. In some instances, the SNP at rs2041739 comprises an “A” or a “G” on a reverse DNA strand encoding the SNP. In some instances, the SNP at rs76362690 comprises an “A” or a “G” on a forward DNA strand encoding the SNP. In some instances, the SNP at rs2287037 comprises an “A” or a “G” on a reverse DNA strand encoding the SNP. In some instances, the SNP at rs80256362 comprises an “A” or a “G” on a forward DNA strand encoding the SNP. In some instances, the IL18R1 risk genotype 1, 2, 3, 4, 5, 6, 7, 8, 9, 10, 11, 12, 13, 14, or 15 SNPs and/or indels. In some instances, if one or more of the above SNPs and/or indels is detected, the subject is “positive” for the IL18R1 risk genotype. In some instances, the kit comprises a pharmaceutical composition comprising a modulator of IL18R1 described herein (e.g., antagonist of IL18R1). In some instances, the subject is administered the antagonist of IL18R1 provided the subject tests “positive” for the IL18R1 risk genotype. In some instances, if one or more of the above SNPs and/or indels is detected, the subject is diagnosed with, or predicted to develop, moderate to severe form of inflammatory bowel disease (IBD, UC, CD), or a subtype of IBD, UC, or CD (e.g., mrUC, stricturing and/or penetrating disease, anti-TNF non-response or loss of response, and the like).

In some instances, the kit is used to determine a level of a gene expression product expressed from a cis gene described herein (e.g. Mitogen-Activated Protein Kinase Kinase Kinase Kinase 4 (MAP4K4), Interleukin 1 Receptor Like 1 (IL1RL1), Transmembrane Protein 182 (TMEM182), and Interleukin 18 Receptor Accessory Protein (IL18RAP), and the like) in a sample obtained from a subject in need thereof. In some instances, the level of the gene expression product detected using the kit described herein is high relative to a normal individual. In some instances, the level of the gene expression product detected using the kit described herein is low relative to a normal individual. In some instances, the subject is administered the agonist of IL18R1 provided the subject has a high or a low expression of the gene expression product. In some instances, if one or more gene expression products is detected, the subject is diagnosed with, or predicted to develop, moderate to severe form of inflammatory bowel disease (IBD, UC, CD), or a subtype of IBD, UC, or CD (e.g., mrUC, stricturing and/or penetrating disease, anti-TNF non-response or loss of response, and the like).

In some instances, the kit is used to detect both the one or more SNPs or indels described above and one or more gene expression product expressed from a cis gene described herein. In some instances, the subject is administered the agonist of IL18R1 provided the subject has a high or a low expression of the gene expression product, and tests “positive” for the IL18R1 risk genotype. In some instances, if the high or low level of the one or more gene expression products is detected and if the subject tests “positive” for the IL18R1 risk genotype, the subject is diagnosed with, or predicted to develop moderate to severe form of inflammatory bowel disease (IBD, UC, CD), or a subtype of IBD, UC, or CD (e.g., mrUC, stricturing and/or penetrating disease, anti-TNF non-response or loss of response, and the like).

Instructions for use may be included in the kit. Optionally, the kit also contains other useful components, such as, diluents, buffers, pharmaceutically acceptable carriers, syringes, catheters, applicators, pipetting or measuring tools, bandaging materials or other useful paraphernalia. The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as compositions and the like. The packaging material is constructed by well-known methods, preferably to provide a sterile, contaminant-free environment. The packaging materials employed in the kit are those customarily utilized in gene expression assays and in the administration of treatments. As used herein, the term “package” refers to a suitable solid matrix or material such as glass, plastic, paper, foil, and the like, capable of holding the individual kit components. Thus, for example, a package can be a glass vial or prefilled syringes used to contain suitable quantities of the pharmaceutical composition. The packaging material has an external label which indicates the contents and/or purpose of the kit and its components.

### Systems

Disclosed herein, in some embodiments, is a system for detecting a particular SNP in IL18R1 in a subject. The system is configured to implement the methods described in this disclosure, including, but not limited to, detecting the presence of a particular CD subtype to determine whether the subject is suitable for treatment with a particular therapy.

In some embodiments, disclosed herein is a system for detecting one or more SNPs in IL18R1 in a subject, comprising: (a) a computer processing device, optionally connected to a computer network; and (b) a software module executed by the computer processing device to analyze a target nucleic acid sequence of one or more IL18R1 risk genotypes in a sample from a subject. In some instances, the one or more IL18R1 risk genotypes comprises rs13001325, rs1420101, rs12479210, rs950880, rs13020553, rs13019081, rs12712141, rs2287037, rs1420102, rs12466380, rs1997467, rs1558619, rs1420088, rs12999364, rs4142132, rs12987977, rs11690443, rs1362350, rs12996505, rs873022, rs974389, rs3771177, rs3732129, rs17026974, rs6706844, rs13020793, rs11685480, rs1558622, rs10183388, rs12712135, rs10189711, rs11685424, rs10189202, rs10191914, rs11123918, rs1968171, rs6733174, rs59247511, rs1558620, rs1921622, rs12998521, rs13017455, rs1362349, rs11123923, rs10190555, rs1035127, rs17027087, rs2080289, rs4851570, rs17027060, rs12712145, rs1420098, rs3732123, rs2287034, rs3860444, rs3821203, rs56258475, rs2270298, rs4851006, rs6710885, rs1568681, rs2241117, rs17027037, rs2270297, rs6753717, rs3755274, rs17027071, rs6750020, rs17027006, rs11683700, rs2058622, rs4851007, rs3732126, rs1807782, rs12469506, rs4851575, rs3771172, rs11465633, rs1135354, rs1558627, rs55927292, rs3771171, rs13015714, rs2160202, rs55883125, rs2041740, rs1035130, rs1420103, rs67723747, rs6543116, rs55664618, rs4851005, rs17027056, rs1420089, rs62152661, rs1420095, rs56030066, rs62152714, rs17696376, rs12105808, rs78248680, rs56151044, rs62152662, rs17651485, rs3771170, rs11123926, rs76721133, rs4988955, rs9807962, rs9808453, rs13424006, rs11695627, rs3771166, rs10173193, rs11465575, rs4851566, rs9308857, rs1974675, rs6751967, rs3771162, rs56386507, rs1997466, rs12712140, and/or rs1362348, or SNP in linkage disequilibrium therewith. In some instances, the system comprises a central processing unit (CPU), memory (e.g., random access memory, flash memory), electronic storage unit, computer program, communication interface to communicate with one or more other systems, and any combination thereof. In some instances, the system is coupled to a computer network, for example, the Internet, intranet, and/or extranet that is in communication with the Internet, a telecommunication, or data network. In some embodiments, the system comprises a storage unit to store data and information regarding any aspect of the methods described in this disclosure. Various aspects of the system are a product or article or manufacture.

One feature of a computer program includes a sequence of instructions, executable in the digital processing device's CPU, written to perform a specified task. In some embodiments, computer readable instructions are implemented as program modules, such as functions, features, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions are combined or distributed as desired in various environments. In some instances, a computer program comprises one sequence of instructions or a plurality of sequences of instructions. A computer program may be provided from one location. A computer program may be provided from a plurality of locations. In some embodiment, a computer program includes one or more software modules. In some embodiments, a computer program includes, in part or in whole, one or more web applications, one or more mobile applications, one or more standalone applications, one or more web browser plug-ins, extensions, add-ins, or add-ons, or combinations thereof

Web Application

In some embodiments, a computer program includes a web application. In light of the disclosure provided herein, those of skill in the art will recognize that a web application may utilize one or more software frameworks and one or more database systems. A web application, for example, is created upon a software framework such as Microsoft® .NET or Ruby on Rails (RoR). A web application, in some instances, utilizes one or more database systems including, by way of non-limiting examples, relational, non-relational, feature oriented, associative, and XML database systems. Suitable relational database systems include, by way of non-limiting examples, Microsoft® SQL Server, mySQL™, and Oracle®. Those of skill in the art will also recognize that a web application may be written in one or more versions of one or more languages. In some embodiments, a web application is written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). A web application may integrate enterprise server products such as IBM® Lotus Domino®. A web application may include a media player element. A media player element may utilize one or more of many suitable multimedia technologies including, by way of non-limiting examples, Adobe® Flash®, HTML 5, Apple® QuickTime®, Microsoft® Silverlight®, Java™, and Unity®.

Mobile Application

In some instances, a computer program includes a mobile application provided to a mobile digital processing device. The mobile application may be provided to a mobile digital processing device at the time it is manufactured. The mobile application may be provided to a mobile digital processing device via the computer network described herein.

A mobile application is created by techniques known to those of skill in the art using hardware, languages, and development environments known to the art. Those of skill in the art will recognize that mobile applications may be written in several languages. Suitable programming languages include, by way of non-limiting examples, C, C++, C#, Featureive-C, Java™, Javascript, Pascal, Feature Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments may be available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry R SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry R App World, App Store for Palm devices, App Catalog for webOS, Windows® Marketplace for Mobile, Ovi Store for Nokia® devices, Samsung® Apps, and Nintendo® DSi Shop.

Standalone Application

In some embodiments, a computer program includes a standalone application, which is a program that may be run as an independent computer process, not an add-on to an existing process, e.g., not a plug-in. Those of skill in the art will recognize that standalone applications are sometimes compiled. In some instances, a compiler is a computer program(s) that transforms source code written in a programming language into binary feature code such as assembly language or machine code. Suitable compiled programming languages include, by way of non-limiting examples, C, C++, Featureive-C, COBOL, Delphi, Eiffel, Java™, Lisp, Python™, Visual Basic, and VB .NET, or combinations thereof. Compilation may be often performed, at least in part, to create an executable program. In some instances, a computer program includes one or more executable complied applications.

Web Browser Plug-in

A computer program, in some aspects, includes a web browser plug-in. In computing, a plug-in, in some instances, is one or more software components that add specific functionality to a larger software application. Makers of software applications may support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. The toolbar may comprise one or more web browser extensions, add-ins, or add-ons. The toolbar may comprise one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB .NET, or combinations thereof.

In some embodiments, Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. The web browser, in some instances, is a mobile web browser. Mobile web browsers (also called mircrobrowsers, mini-browsers, and wireless browsers) may be designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

Software Modules

The medium, method, and system disclosed herein comprise one or more softwares, servers, and database modules, or use of the same. In view of the disclosure provided herein, software modules may be created by techniques known to those of skill in the art using machines, software, and languages known to the art. The software modules disclosed herein may be implemented in a multitude of ways. In some embodiments, a software module comprises a file, a section of code, a programming feature, a programming structure, or combinations thereof. A software module may comprise a plurality of files, a plurality of sections of code, a plurality of programming features, a plurality of programming structures, or combinations thereof. By way of non-limiting examples, the one or more software modules comprise a web application, a mobile application, and/or a standalone application. Software modules may be in one computer program or application. Software modules may be in more than one computer program or application. Software modules may be hosted on one machine. Software modules may be hosted on more than one machine. Software modules may be hosted on cloud computing platforms. Software modules may be hosted on one or more machines in one location. Software modules may be hosted on one or more machines in more than one location.

Databases

The medium, method, and system disclosed herein comprise one or more databases, or use of the same. In view of the disclosure provided herein, those of skill in the art will recognize that many databases are suitable for storage and retrieval of geologic profile, operator activities, division of interest, and/or contact information of royalty owners. Suitable databases include, by way of non-limiting examples, relational databases, non-relational databases, feature oriented databases, feature databases, entity-relationship model databases, associative databases, and XML, databases. In some embodiments, a database is internet-based. In some embodiments, a database is web-based. In some embodiments, a database is cloud computing-based. A database may be based on one or more local computer storage devices.

Data Transmission

The subject matter described herein, including methods for detecting a particular CD subtype, are configured to be performed in one or more facilities at one or more locations. Facility locations are not limited by country and include any country or territory. In some instances, one or more steps are performed in a different country than another step of the method. In some instances, one or more steps for obtaining a sample are performed in a different country than one or more steps for detecting the presence or absence of a particular CD subtype from a sample. In some embodiments, one or more method steps involving a computer system are performed in a different country than another step of the methods provided herein. In some embodiments, data processing and analyses are performed in a different country or location than one or more steps of the methods described herein. In some embodiments, one or more articles, products, or data are transferred from one or more of the facilities to one or more different facilities for analysis or further analysis. An article includes, but is not limited to, one or more components obtained from a subject, e.g., processed cellular material. Processed cellular material includes, but is not limited to, cDNA reverse transcribed from RNA, amplified RNA, amplified cDNA, sequenced DNA, isolated and/or purified RNA, isolated and/or purified DNA, and isolated and/or purified polypeptide. Data includes, but is not limited to, information regarding the stratification of a subject, and any data produced by the methods disclosed herein. In some embodiments of the methods and systems described herein, the analysis is performed and a subsequent data transmission step will convey or transmit the results of the analysis.

In some embodiments, any step of any method described herein is performed by a software program or module on a computer. In additional or further embodiments, data from any step of any method described herein is transferred to and from facilities located within the same or different countries, including analysis performed in one facility in a particular location and the data shipped to another location or directly to an individual in the same or a different country. In additional or further embodiments, data from any step of any method described herein is transferred to and/or received from a facility located within the same or different countries, including analysis of a data input, such as genetic or processed cellular material, performed in one facility in a particular location and corresponding data transmitted to another location, or directly to an individual, such as data related to the diagnosis, prognosis, responsiveness to therapy, or the like, in the same or different location or country.

Business Methods Utilizing a Computer

The methods described herein may utilize one or more computers. The computer may be used for managing customer and sample information such as sample or customer tracking, database management, analyzing molecular profiling data, analyzing cytological data, storing data, billing, marketing, reporting results, storing results, or a combination thereof. The computer may include a monitor or other graphical interface for displaying data, results, billing information, marketing information (e.g. demographics), customer information, or sample information. The computer may also include means for data or information input. The computer may include a processing unit and fixed or removable media or a combination thereof. The computer may be accessed by a user in physical proximity to the computer, for example via a keyboard and/or mouse, or by a user that does not necessarily have access to the physical computer through a communication medium such as a modem, an internet connection, a telephone connection, or a wired or wireless communication signal carrier wave. In some cases, the computer may be connected to a server or other communication device for relaying information from a user to the computer or from the computer to a user. In some cases, the user may store data or information obtained from the computer through a communication medium on media, such as removable media. It is envisioned that data relating to the methods can be transmitted over such networks or connections for reception and/or review by a party. The receiving party can be but is not limited to an individual, a health care provider or a health care manager. In one embodiment, a computer-readable medium includes a medium suitable for transmission of a result of an analysis of a biological sample, such as exosome bio-signatures. The medium can include a result regarding an exosome bio-signature of a subject, wherein such a result is derived using the methods described herein.

The entity obtaining a diagnosis, prognosis, or selecting a patient for a treatment with an antagonist of IL18R1 may enter sample information into a database for the purpose of one or more of the following: inventory tracking, assay result tracking, order tracking, customer management, customer service, billing, and sales. Sample information may include, but is not limited to: customer name, unique customer identification, customer associated medical professional, indicated assay or assays, assay results, adequacy status, indicated adequacy tests, medical history of the individual, preliminary diagnosis, suspected diagnosis, sample history, insurance provider, medical provider, third party testing center or any information suitable for storage in a database. Sample history may include but is not limited to: age of the sample, type of sample, method of acquisition, method of storage, or method of transport.

The database may be accessible by a customer, medical professional, insurance provider, or other third party. Database access may take the form of electronic communication such as a computer or telephone. The database may be accessed through an intermediary such as a customer service representative, business representative, consultant, independent testing center, or medical professional. The availability or degree of database access or sample information, such as assay results, may change upon payment of a fee for products and services rendered or to be rendered. The degree of database access or sample information may be restricted to comply with generally accepted or legal requirements for patient or customer confidentiality.

### Embodiments

1. A method of treating or preventing a disease or condition in a subject, the method comprising administering a modulator of interleukin 18 receptor 1 (IL18R1) activity or expression to the subject, provided the subject has a genotype characterized by the presence of one or more SNPs provided in Tables 1-5.
2. A method of reducing activity or expression of interleukin 18 receptor 1 (IL18R1) in a subject having a genotype characterized by the presence of one or more SNPs provided in Tables 1-5, the method comprising administering to the subject a modulator of IL18R1.
3. The method of embodiment 1 or embodiment 2, further comprising determining the genotype of the subject.
4. The method of embodiment 3, provided that determining the genotype of the subject comprises determining the presence or absence of the one or more SNPs provided Tables 1-5.
5. The method of any previous embodiment, wherein the genotype is detected with an assay comprising polymerase chain reaction (PCR), quantitative reverse-transcription PCR (qPCR), automated sequencing, genotype array, or a combination thereof
6. The method of any previous embodiment, provided that the subject does not comprise the minor allele shown in Tables 1-5.
7. The method of any previous embodiment, provided that the disease or condition is inflammatory bowel disease (IBD).
8. The method of embodiment 7, provided that the IBD comprises Crohn's disease and/or ulcerative colitis.
9. The method of any previous embodiment, provided that the one or more SNPs comprises rs13001325, rs1420101, rs12479210, rs950880, rs13020553, rs13019081, rs12712141, rs2287037, rs1420102, rs12466380, rs1997467, rs1558619, rs1420088, rs12999364, rs4142132, rs12987977, rs11690443, rs1362350, rs12996505, rs873022, rs974389, rs3771177, rs3732129, rs17026974, rs6706844, rs13020793, rs11685480, rs1558622, rs10183388, rs12712135, rs10189711, rs11685424, rs10189202, rs10191914, rs11123918, rs1968171, rs6733174, rs59247511, rs1558620, rs1921622, rs12998521, rs13017455, rs1362349, rs11123923, rs10190555, rs1035127, rs17027087, rs2080289, rs4851570, rs17027060, rs12712145, rs1420098, rs3732123, rs2287034, rs3860444, rs3821203, rs56258475, rs2270298, rs4851006, rs6710885, rs1568681, rs2241117, rs17027037, rs2270297, rs6753717, rs3755274, rs17027071, rs6750020, rs17027006, rs11683700, rs2058622, rs4851007, rs3732126, rs1807782, rs12469506, rs4851575, rs3771172, rs11465633, rs1135354, rs1558627, rs55927292, rs3771171, rs13015714, rs2160202, rs55883125, rs2041740, rs1035130, rs1420103, rs67723747, rs6543116, rs55664618, rs4851005, rs17027056, rs1420089, rs62152661, rs1420095, rs56030066, rs62152714, rs17696376, rs12105808, rs78248680, rs56151044, rs62152662, rs17651485, rs3771170, rs11123926, rs76721133, rs4988955, rs9807962, rs9808453, rs13424006, rs11695627, rs3771166, rs10173193, rs11465575, rs4851566, rs9308857, rs1974675, rs6751967, rs3771162, rs56386507, rs1997466, rs12712140, and rs1362348, or a SNP in linkage disequilibrium therewith, or a combination thereof.
10. The method of any previous embodiment, provided that the one or more SNPs comprises rs13001325.
11. The method of any previous embodiment, provided that the one or more SNPs comprises rs1420101.
12. The method of any previous embodiment, provided that the one or more SNPs comprises rs12479210.
13. The method of any previous embodiment, provided that the one or more SNPs comprises rs950880.
14. The method of any previous embodiment, provided that the one or more SNPs comprises rs13020553.
15. The method of any previous embodiment, provided that the one or more SNPs comprises rs13019081.
16. The method of any previous embodiment, provided that the one or more SNPs comprises rs12712141.
17. The method of any previous embodiment, provided that the one or more SNPs comprises rs2287037.
18. The method of any previous embodiment, provided that the one or more SNPs comprises rs1420102.
19. The method of any previous embodiment, provided that the one or more SNPs comprises rs12466380.
20. The method of any previous embodiment, provided that the one or more SNPs comprises rs1997467.
21. The method of any previous embodiment, provided that the one or more SNPs comprises rs1558619.
22. The method of any previous embodiment, provided that the one or more SNPs comprises rs1420088.
23. The method of any previous embodiment, provided that the one or more SNPs comprises rs12999364.
24. The method of any previous embodiment, provided that the one or more SNPs comprises rs4142132.
25. The method of any previous embodiment, provided that the one or more SNPs comprises rs12987977.
26. The method of any previous embodiment, provided that the one or more SNPs comprises rs11690443.
27. The method of any previous embodiment, provided that the one or more SNPs comprises rs1362350.
28. The method of any previous embodiment, provided that the one or more SNPs comprises rs12996505.
29. The method of any previous embodiment, provided that the one or more SNPs comprises rs873022.
30. The method of any previous embodiment, provided that the one or more SNPs comprises rs974389.
31. The method of any previous embodiment, provided that the one or more SNPs comprises rs3771177.
32. The method of any previous embodiment, provided that the one or more SNPs comprises rs3732129.
33. The method of any previous embodiment, provided that the one or more SNPs comprises rs17026974.
34. The method of any previous embodiment, provided that the one or more SNPs comprises rs6706844.
35. The method of any previous embodiment, provided that the one or more SNPs comprises rs13020793.
36. The method of any previous embodiment, provided that the one or more SNPs comprises rs11685480.
37. The method of any previous embodiment, provided that the one or more SNPs comprises rs1558622.
38. The method of any previous embodiment, provided that the one or more SNPs comprises rs10183388.
39. The method of any previous embodiment, provided that the one or more SNPs comprises rs12712135.
40. The method of any previous embodiment, provided that the one or more SNPs comprises rs10189711.
41. The method of any previous embodiment, provided that the one or more SNPs comprises rs11685424.
42. The method of any previous embodiment, provided that the one or more SNPs comprises rs10189202.
43. The method of any previous embodiment, provided that the one or more SNPs comprises rs10191914.
44. The method of any previous embodiment, provided that the one or more SNPs comprises rs11123918.
45. The method of any previous embodiment, provided that the one or more SNPs comprises rs1968171.
46. The method of any previous embodiment, provided that the one or more SNPs comprises rs6733174.
47. The method of any previous embodiment, provided that the one or more SNPs comprises rs59247511.
48. The method of any previous embodiment, provided that the one or more SNPs comprises rs1558620.
49. The method of any previous embodiment, provided that the one or more SNPs comprises rs1921622.
50. The method of any previous embodiment, provided that the one or more SNPs comprises rs12998521.
51. The method of any previous embodiment, provided that the one or more SNPs comprises rs13017455.
52. The method of any previous embodiment, provided that the one or more SNPs comprises rs1362349.
53. The method of any previous embodiment, provided that the one or more SNPs comprises rs11123923.
54. The method of any previous embodiment, provided that the one or more SNPs comprises rs10190555.
55. The method of any previous embodiment, provided that the one or more SNPs comprises rs1035127.
56. The method of any previous embodiment, provided that the one or more SNPs comprises rs17027087.
57. The method of any previous embodiment, provided that the one or more SNPs comprises rs2080289.
58. The method of any previous embodiment, provided that the one or more SNPs comprises rs4851570.
59. The method of any previous embodiment, provided that the one or more SNPs comprises rs17027060.
60. The method of any previous embodiment, provided that the one or more SNPs comprises rs12712145.
61. The method of any previous embodiment, provided that the one or more SNPs comprises rs1420098.
62. The method of any previous embodiment, provided that the one or more SNPs comprises rs3732123.
63. The method of any previous embodiment, provided that the one or more SNPs comprises rs2287034.
64. The method of any previous embodiment, provided that the one or more SNPs comprises rs3860444.
65. The method of any previous embodiment, provided that the one or more SNPs comprises rs3821203.
66. The method of any previous embodiment, provided that the one or more SNPs comprises rs56258475.
67. The method of any previous embodiment, provided that the one or more SNPs comprises rs2270298.
68. The method of any previous embodiment, provided that the one or more SNPs comprises rs4851006.
69. The method of any previous embodiment, provided that the one or more SNPs comprises rs6710885.
70. The method of any previous embodiment, provided that the one or more SNPs comprises rs1568681.
71. The method of any previous embodiment, provided that the one or more SNPs comprises rs2241117.
72. The method of any previous embodiment, provided that the one or more SNPs comprises rs17027037.
73. The method of any previous embodiment, provided that the one or more SNPs comprises rs2270297.
74. The method of any previous embodiment, provided that the one or more SNPs comprises rs6753717.
75. The method of any previous embodiment, provided that the one or more SNPs comprises rs3755274.
76. The method of any previous embodiment, provided that the one or more SNPs comprises rs17027071.
77. The method of any previous embodiment, provided that the one or more SNPs comprises rs6750020.
78. The method of any previous embodiment, provided that the one or more SNPs comprises rs17027006.
79. The method of any previous embodiment, provided that the one or more SNPs comprises rs11683700.
80. The method of any previous embodiment, provided that the one or more SNPs comprises rs2058622.
81. The method of any previous embodiment, provided that the one or more SNPs comprises rs4851007.
82. The method of any previous embodiment, provided that the one or more SNPs comprises rs3732126.
83. The method of any previous embodiment, provided that the one or more SNPs comprises rs1807782.
84. The method of any previous embodiment, provided that the one or more SNPs comprises rs12469506.
85. The method of any previous embodiment, provided that the one or more SNPs comprises rs4851575.
86. The method of any previous embodiment, provided that the one or more SNPs comprises rs3771172.
87. The method of any previous embodiment, provided that the one or more SNPs comprises rs11465633.
88. The method of any previous embodiment, provided that the one or more SNPs comprises rs1135354.
89. The method of any previous embodiment, provided that the one or more SNPs comprises rs1558627.
90. The method of any previous embodiment, provided that the one or more SNPs comprises rs55927292.
91. The method of any previous embodiment, provided that the one or more SNPs comprises rs3771171.
92. The method of any previous embodiment, provided that the one or more SNPs comprises rs13015714.
93. The method of any previous embodiment, provided that the one or more SNPs comprises rs2160202.
94. The method of any previous embodiment, provided that the one or more SNPs comprises rs55883125.
95. The method of any previous embodiment, provided that the one or more SNPs comprises rs2041740.
96. The method of any previous embodiment, provided that the one or more SNPs comprises rs1035130.
97. The method of any previous embodiment, provided that the one or more SNPs comprises rs1420103.
98. The method of any previous embodiment, provided that the one or more SNPs comprises rs67723747.
99. The method of any previous embodiment, provided that the one or more SNPs comprises rs6543116.
100. The method of any previous embodiment, provided that the one or more SNPs comprises rs55664618.
101. The method of any previous embodiment, provided that the one or more SNPs comprises rs4851005.
102. The method of any previous embodiment, provided that the one or more SNPs comprises rs17027056.
103. The method of any previous embodiment, provided that the one or more SNPs comprises rs1420089.
104. The method of any previous embodiment, provided that the one or more SNPs comprises rs62152661.
105. The method of any previous embodiment, provided that the one or more SNPs comprises rs1420095.
106. The method of any previous embodiment, provided that the one or more SNPs comprises rs56030066.
107. The method of any previous embodiment, provided that the one or more SNPs comprises rs62152714.
108. The method of any previous embodiment, provided that the one or more SNPs comprises rs17696376.
109. The method of any previous embodiment, provided that the one or more SNPs comprises rs12105808.
110. The method of any previous embodiment, provided that the one or more SNPs comprises rs78248680.
111. The method of any previous embodiment, provided that the one or more SNPs comprises rs56151044.
112. The method of any previous embodiment, provided that the one or more SNPs comprises rs62152662.
113. The method of any previous embodiment, provided that the one or more SNPs comprises rs17651485.
114. The method of any previous embodiment, provided that the one or more SNPs comprises rs3771170.
115. The method of any previous embodiment, provided that the one or more SNPs comprises rs11123926.
116. The method of any previous embodiment, provided that the one or more SNPs comprises rs76721133.
117. The method of any previous embodiment, provided that the one or more SNPs comprises rs4988955.
118. The method of any previous embodiment, provided that the one or more SNPs comprises rs9807962.
119. The method of any previous embodiment, provided that the one or more SNPs comprises rs9808453.
120. The method of any previous embodiment, provided that the one or more SNPs comprises rs13424006.
121. The method of any previous embodiment, provided that the one or more SNPs comprises rs11695627.
122. The method of any previous embodiment, provided that the one or more SNPs comprises rs3771166.
123. The method of any previous embodiment, provided that the one or more SNPs comprises rs10173193.
124. The method of any previous embodiment, provided that the one or more SNPs comprises rs11465575.
125. The method of any previous embodiment, provided that the one or more SNPs comprises rs4851566.
126. The method of any previous embodiment, provided that the one or more SNPs comprises rs9308857.
127. The method of any previous embodiment, provided that the one or more SNPs comprises rs1974675.
128. The method of any previous embodiment, provided that the one or more SNPs comprises rs6751967.
129. The method of any previous embodiment, provided that the one or more SNPs comprises rs3771162.
130. The method of any previous embodiment, provided that the one or more SNPs comprises rs56386507.
131. The method of any previous embodiment, provided that the one or more SNPs comprises rs1997466.
132. The method of any previous embodiment, provided that the one or more SNPs comprises rs12712140.
133. The method of any previous embodiment, provided that the one or more SNPs comprises rs1362348.
134. The method of any previous embodiment, provided that the subject has CD.
135. The method of embodiment 134, provided that the one or more SNPs comprises a SNP listed in Table 1.
136. The method of embodiment 134, wherein the single nucleotide polymorphism is associated with structuring.
137. The method of embodiment 136, wherein the stricturing is isolated to an ileocolonic region of an intestine.
138. The method of embodiment 136 or 137, provided that the one or more SNPs comprises a SNP listed in Table 2.
139. The method of embodiment 134, wherein the single nucleotide polymorphism is associated with a risk of a subject developing morphological defects in ileal Paneth cells.
140. The method of embodiment 139, provided that the one or more SNPs comprises a SNP listed in Table 3.
141. The method of any previous embodiment, provided that the subject has IBD.
142. The method of embodiment 141, provided that the one or more SNPs comprises a SNP listed in Table 4.
143. The method of any previous embodiment, provided that the subject has UC.
144. The method of embodiment 143, provided that the one or more SNPs comprises a SNP listed in Table 5.
145. The method of any previous embodiments, provided that the one or more SNPs comprises a SNP listed in FIG. 1A to FIG. 1QQQQQQQ.

### Further Embodiments

1. A computer system for evaluating a biological sample from a subject, the system comprising:
   a) a central computing environment;
   b) an input device operatively connected to said central computing environment, wherein said input device is configured to receive a presence or absence of a genotype that correlates with a disease state in the biological sample;
   c) a trained algorithm executed by said central computing environment, wherein the trained algorithm is configured to use the presence or absence of the genotype to classify said biological sample as a disease or normal sample at an accuracy of at least 85%; and
   d) an output device operatively connected to said central computing environment, wherein said output device is configured to provide information on the classification to a user.
2. The computer system of embodiment 1, wherein the disease state comprises an inflammatory, fibrostenotic, and/or fibrotic disease or condition.
3. The computer system of embodiment 1 or embodiment 2, wherein the disease state comprises inflammatory bowel disease (IBD), Crohn's disease (CD), perianal CD, ulcerative colitis (UC), intestinal fibrosis, pulmonary fibrosis, or intestinal fibrostenosis.
4. The computer system of any previous embodiment, wherein the biological sample comprises whole blood, plasma, serum, or tissue.
5. The computer system of any previous embodiment, wherein the genotype comprises one or more single nucleotide polymorphisms (SNPs) at rs1921622, rs2287037, rs1974675, rs2041739, rs76362690, rs2287037, or rs80256362, a SNP in linkage disequilibrium (LD) therewith, or any combination thereof.
6. The computer system of any previous embodiment, wherein the SNP at rs1921622 comprises an “A” or a “G” on a forward DNA strand encoding the SNP.
7. The computer system of any previous embodiment, wherein the SNP at rs2287037 comprises an “A” or a “G” on a reverse DNA strand encoding the SNP. In some cases, the SNP is rs10213846 and comprises a “G” or a “T” allele.
8. The computer system of any previous embodiment, wherein the SNP at rs1974675 comprises a “C” or a “T” on a reverse DNA strand encoding the SNP.
9. The computer system of any previous embodiment, wherein the SNP at rs2041739 comprises an “A” or a “G” on a reverse DNA strand encoding the SNP.
10. The computer system of any previous embodiment, wherein the SNP at rs76362690 comprises an “A” or a “G” on a forward DNA strand encoding the SNP.
11. The computer system of any previous embodiment, wherein the SNP at rs2287037 comprises an “A” or a “G” on a reverse DNA strand encoding the SNP.
12. The computer system of any previous embodiment, wherein the SNP at rs80256362 comprises an “A” or a “G” on a forward DNA strand encoding the SNP.
13. The computer system of any previous embodiment, wherein the Indel at rs1921622 is within SEQ ID NO: 1.
14. The computer system of any previous embodiment, wherein the SNP at rs2287037 is within SEQ ID NO: 2.
15. The computer system of any previous embodiment, wherein the SNP at rs1974675 is within SEQ ID NO: 3.
16. The computer system of any previous embodiment, wherein the SNP at rs2041739 is within s SEQ ID NO: 4.
17. The computer system of any previous embodiment, wherein the SNP at rs76362690 is within SEQ ID NO: 5.
18. The computer system of any previous embodiment, wherein the SNP at rs2287037 is within SEQ ID NO: 6.
19. The computer system of any previous embodiment, wherein the SNP at rs80256362 is within SEQ ID NO: 7.
20. The computer system of embodiment 5, where LD is defined by an r² value of at least 0.80, 0.85, 0.90, 0.95, or 1.0.
21. The computer system of any previous embodiment, wherein the genotype comprises one or more single nucleotide polymorphisms (SNPs) located at a gene comprising IL18R1.
22. The computer system of any previous embodiment, wherein the genotype is associated with a risk that a subject has, or will develop, inflammatory bowel disease (IBD), Crohn's disease (CD), or ulcerative colitis (UC), as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰.
23. The computer system of any previous embodiment, wherein said output device provides a report summarizing said information on said classification.
24. The computer system of any previous embodiment, wherein said report comprises a recommendation for treatment of said disease state.
25. The computer system of embodiment 24, wherein the treatment comprises administration of a modulator of IL18R1 activity or expression.
26. The computer system of embodiment 25, wherein the modulator of IL18R1 activity or expression comprises an agonist or a partial agonist of IL18R1.
27. The computer system of embodiment 25, wherein the modulator of IL18R1 activity or expression comprises an antagonist or partial antagonist of IL18R1.
28. The computer system of embodiment 26, wherein the agonist or partial agonist comprises an antibody or antigen-binding fragment, peptide, small molecule.
29. The computer system of embodiments 26-27, wherein the modulator of IL18R1 activity or expression comprises an inverse agonist.
30. The computer system of embodiment 25, wherein the modulator of IL18R1 activity or expression comprises a positive allosteric modulator (PAM).
31. The computer system of embodiment 25, wherein the modulator of IL18R1 activity or expression comprises a negative allosteric modulator (NAM).
32. The computer system of embodiments 25-31, wherein the modulator of IL18R1 activity or expression comprises a small molecule that binds to IL18R1 or IL18, or both.
33. The computer system of embodiments 25-31, wherein the modulator of IL18R1 activity or expression comprises an antibody or antigen binding fragment that binds to IL18R1 or IL18, or both.
34. The computer system of embodiments 25-31, wherein the modulator of IL18R1 activity or expression comprises recombinant IL18R1 peptide, or a recombinant IL18 peptide.
35. The computer system of embodiment 25, wherein the modulator of IL18R1 activity or expression comprises a recombinant peptide comprising an amino acid sequence that is about 70%, 75%, 80%, 85%, 90%, 91%, 92%, 93%, 94%, 95%, 96%, 97%, 98%, 99%, or 100% homologous to SEQ ID NO: 8 or SEQ ID NO: 9.
36. The computer system of embodiment 25, wherein the modulator of IL18R1 activity or expression comprises a recombinant peptide comprising an amino acid sequence that is about 70%, 75%, 80%, 85%, 90%, 91%, 92%, 93%, 94%, 95%, 96%, 97%, 98%, 99%, or 100% homologous to SEQ ID NO: 8 or SEQ ID NO: 9, and wherein the amino acid sequence is truncated at the N-terminal and/or C-terminal ends of the peptide.
37. The computer system of embodiments 25-36, wherein the modulator of IL18R1 activity or expression comprises a fusion, a conjugate, or both
38. The computer system of embodiments 25-38, wherein the modulator of IL18R1 activity or expression comprises an agonist or an antagonist of IL18 signaling.
39. The computer system of embodiments 25-38, wherein the modulator of IL18R1 activity or expression comprises an agonist or an antagonist of IL18-IL18R1 binding.
40. The computer system of any preceding embodiment, wherein said genotype is determined with an assay comprising polymerase chain reaction (PCR), quantitative reverse-transcription PCR (qPCR), automated sequencing, genotype array, or a combination thereof
41. Use of a composition comprising one or more binding agents for generating a report that classifies a biological sample from as subject as disease or non-disease, wherein the one or more binding agents specifically bind to one or more polymorphisms of one or more genes selected from MAP4K4, IL1RL1, TMEM182, and IL18RAP, or their complement.
42. The use of embodiment 41, wherein generating the report further comprises:
   (a) providing the biological sample from the subject;
   (b) assaying the biological sample from the subject for detecting the presence of the one or more polymorphisms of the one or more genes;
   (c) generating the report based on the result of step (b); and
   (d) determining whether said subject has or is likely to have the disease based on the results of step (b).
43. The use of embodiment 41 or 42, wherein the disease state comprises an inflammatory, fibrostenotic, and/or fibrotic disease or condition.
44. The use of embodiment 41 or embodiment 42, wherein the disease state comprises inflammatory bowel disease (IBD), Crohn's disease (CD), perianal CD, ulcerative colitis (UC), intestinal fibrosis, pulmonary fibrosis, or intestinal fibrostenosis.
45. The use of any of embodiments 41-44, wherein the biological sample comprises whole blood, plasma, serum, or tissue.
46. The use of embodiment 42, wherein the assaying step (b) comprises: (a) contacting the biological sample with the one or more binding agents that specifically bind to the one or more polymorphisms; and (b) determining whether the biological sample specifically binds to said one or more binding agents, wherein binding of the biological sample to the one or more binding agents indicates the presence of the polymorphism in the subject.
47. The use of embodiment 46, wherein the detecting assaying step (b) comprises sequencing of the biological sample.
48. The use of embodiment 46, wherein the detecting assaying step (b) comprises quantifying the amount of DNA comprising the one or more polymorphs.
49. The use of embodiment 48, wherein the quantifying comprises PCR.
50. The use of embodiment 49, wherein the PCR comprises real-time PCR.
51. The use of embodiment 48, wherein the quantifying comprises hybridization.
52. A composition comprising one or more binding agents that specifically bind to the one or more polymorphisms of one or more genes selected from MAP4K4, IL1RL1, TMEM182, and IL18RAP, or their complement, wherein the one or more binding agents are selected to classify a biological sample as disease or non-disease.
53. The composition of embodiment 52, wherein the one or more binding agents comprise oligonucleotides.
54. The composition of embodiment 53, wherein the oligonucleotides comprise RNA or DNA.
55. The composition of embodiment 52, wherein the one or more binding agents comprise aptamers, antibodies, peptide nucleic acids, or pyranosyl RNA.
56. The composition of embodiment 52, wherein the one or more polymorphisms comprise deletions or duplications.
57. A kit for diagnosing detecting a disease or condition in a subject, the kit comprising: (a) at least one binding agent that specifically binds to the one or more polymorphisms of one or more genes selected from the group consisting of MAP4K4, IL1RL1, TMEM182, and IL18RAP, or their complement, wherein the at least one binding agent is selected to detect a disease or non-disease state; and (b) reagents for detecting binding of said at least one binding agent to a DNA sample from a subject.
58. The kit of embodiment 57, wherein the composition comprises binding agents for at most 10,000 genes.
59. The kit of embodiment 57, wherein the at least one binding agent comprises at least one oligonucleotide.
60. The kit of embodiment 57, wherein the at least one binding agent comprises at least one aptamer, antibody, peptide nucleic acid, or pyranosyl RNA.
61. The kit of embodiment 57, wherein the at least one binding agent is labelled with a detectable label.
62. The kit of embodiment 57, wherein the at least one binding agent is immobilized to a surface.
63. A system for generating a report that classifies a biological sample a disease or non-disease, comprising: (a) a computer system that (i) generates a molecular profile of a DNA sample based upon the presence of one or more polymorphisms of one or more genes selected from MAP4K4, IL1RL1, TMEM182, and IL18RAP, or their complement, and (ii) generates a report that classifies the biological sample based on said molecular profile; and (b) a computer screen that displays said report.
64. The system of embodiment 63, wherein the presence of one or more polymorphisms is based on the result of an assay of said DNA sample, which result is entered into a database.
65. The system of embodiment 64, further comprising an input for said result.

### EXAMPLES

### Example 1. IL18R1 SNPs Associated with Crohn's Disease

Subjects were recruited and diagnosed as having or not having inflammatory bowel disease (IBD). Some IBD patients were further characterized as having Crohn's disease (CD) or ulcerative colitis (UC). Diagnosis was based on standard endoscopic, histologic, and/or radiographic features. Select subjects were further characterized based on genetic and/or phenotypic traits.

Blood samples were collected from the subjects at the time of enrollment. Genotyping was performed on the samples using Illumina ImmunoChip (Illumina, San Diego, Calif.) per manufacturer's protocol. Markers were excluded from analysis based on: Hardy-Weinberg Equilibrium p≤1.0E-5; genotyping rate <95%; minor allele frequency <1%. Related individuals (Pi-hat scores >0.25) were identified using identity-by-descent and excluded from analysis (PLINK). Admixture was used to generate ethnicity proportion estimations for all individuals. Only subjects identified by admixture as Caucasian (proportion <0.75) were included in the analysis.

Multiple large-scale case-control association studies involving CD, UC, and IBD, and subclinical phenotypes of CD, UC, and IBD, in populations using gene-based single nucleotide polymorphism (SNP) markers were performed. The studies included subjects recruited at the Cedars-Sinai Inflammatory Bowel Disease Centers. The studies also included GWAS data derived from the International Inflammatory Bowel Diseases Genetic Consortium (IIBDGC) and cohorts from DeLange et al., “Genome-wide association study implicates immune activation of multiple integrin genes in inflammatory bowel disease,” Nature Genetics. Vol. 49, No. 2 (February 2017). Results showed that SNPs at the IL18R1 locus provided in Table 1 are significantly associated with Crohn's Disease.

Genotyping data was produced from tissue samples from patients diagnosed with IBD and healthy patients without IBD. eQTL mapping was performed on these samples. Transcriptomic data was generated on the tissue. Briefly, uninflamed tissue from formalin-fixed paraffin-embedded (FFPE) resection margins of subjects requiring surgery at Cedars-Sinai Medical Center for Crohn's disease was identified. Whole-thickness ileal tissue was scraped from the FFPE tissue sections followed by RNA extraction using the RNeasy FFPE kit (Qiagen) according to the manufacturer's instructions. The Transplex Whole Transcriptome Amplification kit (WTA2; Sigma) was used for cDNA synthesis and amplification. Subsequent purification of the cDNA product was performed with the PCR Purification kit (Qiagen). Sample quality was confirmed using the Agilent Bioanalyzer. For samples passing quality control, Cy5 labeling with the ULST Fluorescent Labeling kit (Kreatech) and hybridization (performed in duplicate for each sample) to Whole Human Genome 4×44k Microarrays (Agilent) was performed.

Single channel microarray expression data extracted using Agilent feature extraction software was received from Genome Technology Access Center at Washington University in St. Louis. Raw expression data available in technical duplicates was normalized using LIMMA package implemented in R version 3.2.2. The expression data preprocessing included background correction of the expression data, followed by log 2-transformation and quantile-normalization. Unsupervised hierarchical clustering of expression data was used to remove outlier subjects. eQTL mapping was implemented in Matrix eQTL R package using the available expression and genotype data from patients. Associations between genotype and probe expression level were performed using a linear regression model with additive genotype effects. All associations were adjusted for gender, age and population sub-structure using the first two principal components. Gene bounds were defined using a 1 Mb window around the transcription start position of given gene as obtained from UCSC Genome Browser. For cis-eQTL mapping, a 1 Mb cis distance from gene bounds was used. Cis-eQTLs were defined as association signals from SNPs located within 1 Mb from each of the gene bounds. False discovery rates (FDR) were estimated to correct for multiple testing using Matrix eQTL according to the Benjamini and Hochberg method. Note that FDR calculation in matrix eQTL does not take into account “linkage disequilibrium” between the SNPs and may be overly stringent. A negative beta value indicates a decrease in IL18R1 gene expression. A positive beta value indicates an increase in IL18R1 gene expression.

Tables 1-5 provide meta-analysis of SNPs and/or indels considered predictive of disease (IBD, CD, UC), phenotype, and/or suitability to treatment with a modulator of IL18R1 (p value cutoff of 1.0 E-3). FIG. 1A to FIG. 1QQQQQQQ provides the entire meta-analysis, without p value cutoff. These results show strong associations between the SNPs listed in the tables, and the associated diseases (IBD, CD, UC) and phenotypes. The results also show that particular SNPs are associated with decreased or increased expression of IL18R1, MAP4K4, IL1RL1, TMEM182, and IL18RAP in patients diagnosed with IBD, CD, or UC having the provided phenotypes. Table 6 and Table 7 provide a description of the headers of Tables 1-5, and FIG. 1A to FIG. 1QQQQQQQ.

To determine Paneth genotype, genotyping data was collected from patients with Crohn's Disease (CD) with morphological defects of ileal Paneth cells, as determined using the classification set forth in VanDussen et al., “Genetic Variants Synthesize the Produce Paneth Cell Phenotypes That Define Subtypes of Crohn's Disease,” Gastroenterology 2014; 146:200-209.

TABLE 1
IL18R1 SNPs Significantly Correlated with Crohn's disease (CD)

| PHENO- | | | | | n_ | OR_ | base_pair_ | cis_ | | | |
|---|---|---|---|---|---|---|---|---|---|---|---|
| TYPE | Marker | dbSNP | p_value | A1 | miss | Z_B | CH2 | eGENE | entl_beta | entl_p | priority |
| CD vs. | imm_2_ | rs13001325 | <1.0E−3 | A | 94040 | −0.1236 | 102939036 | NA | NA | NA | NA |
| Ctrl | 102305468 | | | | | | | | | | |
| CD vs. | imm_2_ | rs1420101 | <1.0E−3 | A | 94080 | −0.1230 | 102957716 | NA | NA | NA | NA |
| Ctrl | 102324148 | | | | | | | | | | |
| CD vs. | imm_2_ | rs12479210 | <1.0E−3 | A | 94088 | −0.1226 | 102949161 | NA | NA | NA | NA |
| Ctrl | 102315593 | | | | | | | | | | |
| CD vs. | imm_2_ | rs950880 | <1.0E−3 | A | 94092 | −0.1212 | 102932562 | NA | NA | NA | NA |
| Ctrl | 102298994 | | | | | | | | | | |
| CD vs. | imm_2_ | rs13020553 | <1.0E−3 | G | 94088 | −0.1213 | 102931826 | NA | NA | NA | NA |
| Ctrl | 102298258 | | | | | | | | | | |
| CD vs. | imm_2_ | rs13019081 | <1.0E−3 | C | 91372 | −0.1206 | 102950822 | NA | NA | NA | NA |
| Ctrl | 102317254 | | | | | | | | | | |
| CD vs. | imm_2_ | rs12712141 | <1.0E−3 | A | 94101 | 0.1112 | 102953067 | NA | NA | NA | NA |
| Ctrl | 102319499 | | | | | | | | | | |
| CD vs. | imm_2_ | rs2287037 | <1.0E−3 | A | 90448 | −0.1179 | 102979028 | NA | NA | NA | NA |
| Ctrl | 102345460 | | | | | | | | | | |
| CD vs. | imm_2_ | rs1420102 | <1.0E−3 | G | 94059 | 0.1104 | 102948819 | NA | NA | NA | NA |
| Ctrl | 102315251 | | | | | | | | | | |
| CD vs. | imm_2_ | rs12466380 | <1.0E−3 | A | 94097 | 0.1100 | 102948939 | NA | NA | NA | NA |
| Ctrl | 102315371 | | | | | | | | | | |
| CD vs. | imm_2_ | rs1997467 | <1.0E−3 | A | 94088 | 0.1100 | 102951073 | NA | NA | NA | NA |
| Ctrl | 102317505 | | | | | | | | | | |
| CD vs. | imm_2_ | rs1558619 | <1.0E−3 | C | 94094 | 0.1099 | 102931550 | NA | NA | NA | NA |
| Ctrl | 102297982 | | | | | | | | | | |
| CD vs. | imm_2_ | rs1420088 | <1.0E−3 | A | 94092 | 0.1099 | 102939434 | NA | NA | NA | NA |
| Ctrl | 102305866 | | | | | | | | | | |
| CD vs. | imm_2_ | rs12999364 | <1.0E−3 | A | 94085 | −0.1139 | 102974129 | NA | NA | NA | NA |
| Ctrl | 102340561 | | | | | | | | | | |
| CD vs. | imm_2_ | rs4142132 | <1.0E−3 | G | 94098 | 0.1096 | 102937482 | NA | NA | NA | NA |
| Ctrl | 102303914 | | | | | | | | | | |
| CD vs. | imm_2_ | rs12987977 | <1.0E−3 | C | 94101 | −0.1136 | 102975336 | NA | NA | NA | NA |
| Ctrl | 102341768 | | | | | | | | | | |
| CD vs. | imm_2_ | rs11690443 | <1.0E−3 | T | 94088 | 0.1095 | 102936131 | NA | NA | NA | NA |
| Ctrl | 102302563 | | | | | | | | | | |
| CD vs. | imm_2_ | rs1362350 | <1.0E−3 | G | 93070 | 0.1104 | 102951798 | NA | NA | NA | NA |
| Ctrl | 102318230 | | | | | | | | | | |
| CD vs. | imm_2_ | rs12996505 | <1.0E−3 | A | 94097 | 0.1092 | 102931802 | NA | NA | NA | NA |
| Ctrl | 102298234 | | | | | | | | | | |
| CD vs. | imm_2_ | rs873022 | <1.0E−3 | A | 94097 | −0.1245 | 102955683 | NA | NA | NA | NA |
| Ctrl | 102322115 | | | | | | | | | | |
| CD vs. | imm_2_ | rs974389 | <1.0E−3 | G | 94098 | 0.1090 | 102936981 | NA | NA | NA | NA |
| Ctrl | 102303413 | | | | | | | | | | |
| CD vs. | imm_2_ | rs3771177 | <1.0E−3 | A | 94098 | −0.1244 | 102955860 | NA | NA | NA | NA |
| Ctrl | 102322292 | | | | | | | | | | |
| CD vs. | imm_2_ | rs3732129 | <1.0E−3 | G | 94095 | −0.1243 | 102957532 | NA | NA | NA | NA |
| Ctrl | 102323964 | | | | | | | | | | |
| CD vs. | imm_2_ | rs17026974 | <1.0E−3 | A | 94079 | −0.1243 | 102952360 | NA | NA | NA | NA |
| Ctrl | 102318792 | | | | | | | | | | |
| CD vs. | imm_2_ | rs6706844 | <1.0E−3 | A | 94078 | 0.1086 | 102940412 | NA | NA | NA | NA |
| Ctrl | 102306844 | | | | | | | | | | |
| CD vs. | imm_2_ | rs13020793 | <1.0E−3 | G | 94087 | 0.1086 | 102931926 | NA | NA | NA | NA |
| Ctrl | 102298358 | | | | | | | | | | |
| CD vs. | imm_2_ | rs11685480 | <1.0E−3 | G | 94076 | 0.1084 | 102927086 | NA | NA | NA | NA |
| Ctrl | 102293518 | | | | | | | | | | |
| CD vs. | imm_2_1 | rs1558622 | <1.0E−3 | G | 94067 | 0.1083 | 102930147 | NA | NA | NA | NA |
| Ctrl | 02296579 | | | | | | | | | | |
| CD vs. | imm_2_ | rs10183388 | <1.0E−3 | G | 94099 | 0.1082 | 102932247 | NA | NA | NA | NA |
| Ctrl | 102298679 | | | | | | | | | | |
| CD vs. | imm_2_ | rs12712135 | <1.0E−3 | A | 94095 | 0.1081 | 102930948 | NA | NA | NA | NA |
| Ctrl | 102297380 | | | | | | | | | | |
| CD vs. | imm_2_ | rs10189711 | <1.0E−3 | A | 94093 | 0.1080 | 102930881 | NA | NA | NA | NA |
| Ctrl | 102297313 | | | | | | | | | | |
| CD vs. | imm_2_ | rs11685424 | <1.0E−3 | G | 94097 | 0.1079 | 102926981 | NA | NA | NA | NA |
| Ctrl | 102293413 | | | | | | | | | | |
| CD vs. | imm_2_ | rs10189202 | <1.0E−3 | A | 94101 | 0.1078 | 102930380 | NA | NA | NA | NA |
| Ctrl | 102296812 | | | | | | | | | | |
| CD vs. | imm_2_ | rs10191914 | <1.0E−3 | A | 94098 | 0.1078 | 102930657 | NA | NA | NA | NA |
| Ctrl | 102297089 | | | | | | | | | | |
| CD vs. | imm_2_ | rs11123918 | <1.0E−3 | A | 93761 | 0.1081 | 102935237 | NA | NA | NA | NA |
| Ctrl | 102301669 | | | | | | | | | | |
| CD vs. | imm_2_ | rs1968171 | <1.0E−3 | NA | NA | 0.1121 | 102933552 | NA | NA | NA | NA |
| Ctrl | 102299984 | | | | | | | | | | |
| CD vs. | imm_2_ | rs6733174 | <1.0E−3 | A | 94099 | 0.1077 | 102929012 | NA | NA | NA | NA |
| Ctrl | 102295444 | | | | | | | | | | |
| CD vs. | imm_2_ | rs59247511 | <1.0E−3 | G | 88756 | −0.1102 | 102954190 | NA | NA | NA | NA |
| Ctrl | 102320622 | | | | | | | | | | |
| CD vs. | imm_2_ | rs1558620 | <1.0E−3 | A | 89819 | 0.1088 | 102931395 | NA | NA | NA | NA |
| Ctrl | 102297827 | | | | | | | | | | |
| CD vs. | imm_2_ | rs1921622 | <1.0E−3 | G | 94090 | 0.1070 | 102966067 | MAP- | 0.0496 | 0.0167 | 0 |
| Ctrl | 102332499 | | | | | | | 4K4 | | | |
| CD vs. | rs12998521 | rs12998521 | <1.0E−3 | A | 92721 | −0.1116 | 102974417 | NA | NA | NA | NA |
| Ctrl | | | | | | | | | | | |
| CD vs. | imm_2_ | rs13017455 | <1.0E−3 | A | 91754 | −0.1103 | 102964742 | NA | NA | NA | NA |
| Ctrl | 102331174 | | | | | | | | | | |
| CD vs. | imm_2_ | rs1362349 | <1.0E−3 | C | 87425 | −0.1071 | 102951972 | NA | NA | NA | NA |
| Ctrl | 102318404 | | | | | | | | | | |
| CD vs. | imm_2_ | rs11123923 | <1.0E−3 | A | 94070 | −0.1068 | 102967844 | NA | NA | NA | NA |
| Ctrl | 102334276 | | | | | | | | | | |
| CD vs. | imm_2_ | rs10190555 | <1.0E−3 | A | 93178 | 0.1198 | 102994056 | NA | NA | NA | NA |
| Ctrl | 102360488 | | | | | | | | | | |
| CD vs. | imm_2_ | rs1035127 | <1.0E−3 | A | 94060 | 0.1189 | 103019919 | NA | NA | NA | NA |
| Ctrl | 102386351 | | | | | | | | | | |
| CD vs. | imm_2_ | rs17027087 | <1.0E−3 | A | 94100 | −0.1165 | 103015918 | IL1RL1 | −0.0673 | 0.0437 | 0 |
| Ctrl | 102382350 | | | | | | | | | | |
| CD vs. | imm_2_ | rs2080289 | <1.0E−3 | A | 94058 | −0.1164 | 102995020 | IL1RL1 | −0.0673 | 0.0437 | 0 |
| Ctrl | 102361452 | | | | | | | | | | |
| CD vs. | imm_2_ | rs4851570 | <1.0E−3 | G | 94084 | −0.1161 | 103006387 | IL1RL1 | −0.0673 | 0.0437 | 0 |
| Ctrl | 102372819 | | | | | | | | | | |
| CD vs. | imm_2_ | rs17027060 | <1.0E−3 | G | 94097 | −0.1158 | 103007567 | IL1RL1 | −0.0673 | 0.0437 | 0 |
| Ctrl | 102373999 | | | | | | | | | | |
| CD vs. | imm_2_ | rs12712145 | <1.0E−3 | A | 94090 | 0.1178 | 103008710 | NA | NA | NA | NA |
| Ctrl | 102375142 | | | | | | | | | | |
| CD vs. | imm_2_ | rs1420098 | <1.0E−3 | G | 86682 | −0.1084 | 102984279 | NA | NA | NA | NA |
| Ctrl | 102350711 | | | | | | | | | | |
| CD vs. | imm_2_ | rs3732123 | <1.0E−3 | G | 94095 | −0.1159 | 103018077 | IL1RL1 | −0.0673 | 0.0437 | 0 |
| Ctrl | 102384509 | | | | | | | | | | |
| CD vs. | imm_2_ | rs2287034 | <1.0E−3 | A | 94095 | −0.1156 | 103010588 | IL1RL1 | −0.0673 | 0.0437 | 0 |
| Ctrl | 102377020 | | | | | | | | | | |
| CD vs. | imm_2_ | rs3860444 | <1.0E−3 | A | 94084 | 0.1172 | 103007623 | NA | NA | NA | NA |
| Ctrl | 102374055 | | | | | | | | | | |
| CD vs. | imm_2_ | rs3821203 | <1.0E−3 | A | 94040 | −0.1155 | 102996872 | IL1RL1 | −0.0673 | 0.0437 | 0 |
| Ctrl | 102363304 | | | | | | | | | | |
| CD vs. | imm_2_ | rs56258475 | <1.0E−3 | G | 94064 | −0.1154 | 102999312 | IL1RL1 | −0.0673 | 0.0437 | 0 |
| Ctrl | 102365744 | | | | | | | | | | |
| CD vs. | imm_2_ | rs2270298 | <1.0E−3 | G | 94101 | −0.1153 | 102992079 | IL1RL1 | −0.0673 | 0.0437 | 0 |
| Ctrl | 102358511 | | | | | | | | | | |
| CD vs. | imm_2_ | rs4851006 | <1.0E−3 | A | 93111 | −0.1164 | 103024738 | NA | NA | NA | NA |
| Ctrl | 102391170 | | | | | | | | | | |
| CD vs. | imm_2_ | rs6710885 | <1.0E−3 | G | 87495 | −0.1078 | 102977537 | NA | NA | NA | NA |
| Ctrl | 102343969 | | | | | | | | | | |
| CD vs. | imm_2_ | rs1568681 | <1.0E−3 | G | 94078 | 0.1169 | 103014696 | NA | NA | NA | NA |
| Ctrl | 102381128 | | | | | | | | | | |
| CD vs. | imm_2_ | rs2241117 | <1.0E−3 | A | 94101 | 0.1168 | 103003043 | NA | NA | NA | NA |
| Ctrl | 102369475 | | | | | | | | | | |
| CD vs. | imm_2_ | rs17027037 | <1.0E−3 | G | 94084 | −0.1150 | 102994884 | IL1RL1 | −0.0673 | 0.0437 | 0 |
| Ctrl | 102361316 | | | | | | | | | | |
| CD vs. | imm_2_ | rs2270297 | <1.0E−3 | A | 94087 | 0.1167 | 102992675 | NA | NA | NA | NA |
| Ctrl | 102359107 | | | | | | | | | | |
| CD vs. | imm_2_ | rs6753717 | <1.0E−3 | A | 94088 | 0.1167 | 102993161 | NA | NA | NA | NA |
| Ctrl | 102359593 | | | | | | | | | | |
| CD vs. | imm_2_ | rs3755274 | <1.0E−3 | A | 94096 | 0.1165 | 103002395 | NA | NA | NA | NA |
| Ctrl | 102368827 | | | | | | | | | | |
| CD vs. | imm_2_ | rs17027071 | <1.0E−3 | A | 94100 | −0.1148 | 103012674 | IL1RL1 | −0.0673 | 0.0437 | 0 |
| Ctrl | 102379106 | | | | | | | | | | |
| CD vs. | imm_2_ | rs6750020 | <1.0E−3 | G | 94092 | 0.1165 | 102994714 | NA | NA | NA | NA |
| Ctrl | 102361146 | | | | | | | | | | |
| CD vs. | imm_2_ | rs17027006 | <1.0E−3 | C | 94096 | −0.1150 | 102965332 | IL1RL1 | −0.0663 | 0.0409 | IL1RL1 |
| Ctrl | 102331764 | | | | | | | | | | |
| CD vs. | imm_2_ | rs11683700 | <1.0E−3 | A | 94100 | −0.1146 | 102996805 | IL1RL1 | −0.0673 | 0.0437 | 0 |
| Ctrl | 102363237 | | | | | | | | | | |
| CD vs. | imm_2_ | rs2058622 | <1.0E−3 | A | 94099 | 0.1166 | 102985424 | NA | NA | NA | NA |
| Ctrl | 102351856 | | | | | | | | | | |
| CD vs. | imm_2_ | rs4851007 | <1.0E−3 | A | 94090 | 0.1162 | 103024813 | NA | NA | NA | NA |
| Ctrl | 102391245 | | | | | | | | | | |
| CD vs. | imm_2_ | rs3732126 | <1.0E−3 | C | 89806 | −0.1162 | 103013962 | IL1RL1 | −0.0673 | 0.0437 | 0 |
| Ctrl | 102380394 | | | | | | | | | | |
| CD vs. | imm_2_ | rs1807782 | <1.0E−3 | G | 94057 | 0.1159 | 103033147 | NA | NA | NA | NA |
| Ctrl | 102399579 | | | | | | | | | | |
| CD vs. | imm_2_ | rs12469506 | <1.0E−3 | A | 94099 | −0.1139 | 102965871 | IL1RL1 | −0.0659 | 0.0477 | IL1RL1 |
| Ctrl | 102332303 | | | | | | | | | | |
| CD vs. | imm_2_ | rs4851575 | <1.0E−3 | G | 93053 | 0.1164 | 103025203 | NA | NA | NA | NA |
| Ctrl | 102391635 | | | | | | | | | | |
| CD vs. | imm_2_ | rs3771172 | <1.0E−3 | A | 94093 | −0.1129 | 102985812 | NA | NA | NA | NA |
| Ctrl | 102352244 | | | | | | | | | | |
| CD vs. | imm_2_ | rs11465633 | <1.0E−3 | A | 93025 | −0.1139 | 102997733 | NA | NA | NA | NA |
| Ctrl | 102364165 | | | | | | | | | | |
| CD vs. | imm_2_ | rs1135354 | <1.0E−3 | C | 94075 | −0.1127 | 103014302 | IL1RL1 | −0.0673 | 0.0437 | 0 |
| Ctrl | 102380734 | | | | | | | | | | |
| CD vs. | imm_2_ | rs1558627 | <1.0E−3 | G | 92154 | 0.1166 | 102984684 | NA | NA | NA | NA |
| Ctrl | 102351116 | | | | | | | | | | |
| CD vs. | imm_2_ | rs55927292 | <1.0E−3 | A | 94066 | −0.1129 | 102964861 | IL1RL1 | −0.0700 | 0.0305 | IL1RL1 |
| Ctrl | 102331293 | | | | | | | | | | |
| CD vs. | imm_2_ | rs3771171 | <1.0E−3 | G | 93096 | −0.1132 | 102985950 | NA | NA | NA | NA |
| Ctrl | 102352382 | | | | | | | | | | |
| CD vs. | imm_2_ | rs13015714 | <1.0E−3 | C | 94091 | 0.1140 | 102971865 | NA | NA | NA | NA |
| Ctrl | 102338297 | | | | | | | | | | |
| CD vs. | imm_2_ | rs2160202 | <1.0E−3 | A | 93116 | −0.1130 | 102986154 | NA | NA | NA | NA |
| Ctrl | 102352586 | | | | | | | | | | |
| CD vs. | imm_2_ | rs55883125 | <1.0E−3 | A | 89792 | −0.1132 | 103024331 | IL1RL1 | −0.0673 | 0.0437 | 0 |
| Ctrl | 102390763 | | | | | | | | | | |
| CD vs. | imm_2_ | rs2041740 | <1.0E−3 | A | 89771 | 0.1128 | 102989734 | NA | NA | NA | NA |
| Ctrl | 102356166 | | | | | | | | | | |
| CD vs. | imm_2_ | rs1035130 | <1.0E−3 | A | 87767 | −0.1101 | 103001402 | NA | NA | NA | NA |
| Ctrl | 102367834 | | | | | | | | | | |
| CD vs. | imm_2_ | rs1420103 | <1.0E−3 | A | 94095 | 0.1050 | 102948632 | NA | NA | NA | NA |
| Ctrl | 102315064 | | | | | | | | | | |
| CD vs. | imm_2_ | rs67723747 | <1.0E−3 | NA | NA | −0.1089 | 102969807 | NA | NA | NA | NA |
| Ctrl | 102336239 | | | | | | | | | | |
| CD vs. | imm_2_ | rs6543116 | <1.0E−3 | A | 94078 | 0.1020 | 102927726 | NA | NA | NA | NA |
| Ctrl | 102294158 | | | | | | | | | | |
| CD vs. | imm_2_ | rs55664618 | <1.0E−3 | A | 94101 | −0.0865 | 103016216 | IL1RL1 | −0.0559 | 0.0418 | 0 |
| Ctrl | 102382648 | | | | | | | | | | |
| CD vs. | imm_2_ | rs4851005 | <1.0E−3 | A | 94039 | −0.0752 | 103011552 | NA | NA | NA | NA |
| Ctrl | 102377984 | | | | | | | | | | |
| CD vs. | imm_2_ | rs17027056 | <1.0E−3 | A | 94095 | −0.1052 | 103007051 | NA | NA | NA | NA |
| Ctrl | 102373483 | | | | | | | | | | |
| CD vs. | imm_2_ | rs1420089 | <1.0E−3 | G | 94092 | 0.0653 | 102938389 | Th1R2, | 0.231497338126034, | 0.0403730838316627, | 0, 0 |
| Ctrl | 102304821 | | | | | | | IL1RL2 | 0.122966178554851 | 0.0456541874537971 | |
| CD vs. | imm_2_ | rs62152661 | <1.0E−3 | G | 94094 | 0.0636 | 102959646 | Th1R2, | 0.231497338126034, | 0.0403730838316627, | 0, 0 |
| Ctrl | 102326078 | | | | | | | IL1RL2 | 0.122966178554851 | 0.0456541874537971 | |
| CD vs. | imm_2_ | rs1420095 | <1.0E−3 | G | 94076 | 0.0691 | 103012902 | Th1R1, | 0.0838534335200365, | 0.00372427407991006, | 0, 0, 0, |
| Ctrl | 102379334 | | | | | | | IL18- | 0.11811725329138, | 0.0200804208351232, | 0, 0 |
| | | | | | | | | RAP, | −0.231864788573836, | 0.0239002404371004, | |
| | | | | | | | | RPL31, | 0.170724364161569, | 0.0257519329018087, | |
| | | | | | | | | IL1RL2, | −0.263402645000446 | 0.0334284392287019 | |
| | | | | | | | | RPL31 | | | |
| CD vs. | imm_2_ | rs56030066 | <1.0E−3 | C | 94101 | 0.0688 | 103003034 | Th1R1, | 0.0838534335200365, | 0.00372427407991006, | 0, 0, 0, |
| Ctrl | 102369466 | | | | | | | IL18- | 0.11811725329138, | 0.0200804208351232, | 0, 0 |
| | | | | | | | | RAP, | −0.231864788573836, | 0.0239002404371004, | |
| | | | | | | | | RPL31, | 0.170724364161569, | 0.0257519329018087, | |
| | | | | | | | | IL1RL2, | −0.263402645000446 | 0.0334284392287019 | |
| | | | | | | | | RPL31 | | | |
| CD vs. | imm_2_ | rs62152714 | <1.0E−3 | G | 94099 | 0.0668 | 103032726 | Th1R1, | 0.0838534335200365, | 0.00372427407991006, | 0, |
| Ctrl | 102399158 | | | | | | | IL18- | 0.11811725329138, | 0.0200804208351232, | IL18- |
| | | | | | | | | RAP, | −0.231864788573836, | 0.0239002404371004, | RAP, |
| | | | | | | | | RPL31, | 0.170724364161569, | 0.0257519329018087, | 0, 0, 0 |
| | | | | | | | | IL1RL2, | −0.263402645000446 | 0.0334284392287019 | |
| | | | | | | | | RPL31 | | | |
| CD vs. | imm_2_ | rs17696376 | <1.0E−3 | A | 94096 | 0.0625 | 102965153 | Th1R1, | 0.0496830463208274, | 0.0406804596047685, | 0, 0, 0 |
| Ctrl | 102331585 | | | | | | | Th1R2, | 0.237344378396056, | 0.0430463438168161, | |
| | | | | | | | | IL1RL2 | 0.126970254426939 | 0.0469360657670677 | |
| CD vs. | imm_2_ | rs12105808 | <1.0E−3 | T | 94095 | 0.0604 | 102974222 | IL1R1, | 0.0496830463208274, | 0.0406804596047685, | 0, 0, 0 |
| Ctrl | 102340654 | | | | | | | IL1R2, | 0.237344378396056, | 0.0430463438168161, | |
| | | | | | | | | IL1RL2 | 0.126970254426939 | 0.0469360657670677 | |
| CD vs. | imm_2_ | rs78248680 | <1.0E−3 | G | 94094 | 0.0599 | 102968670 | IL1R1, | 0.0496830463208274, | 0.0406804596047685, | 0, 0, 0 |
| Ctrl | 102335102 | | | | | | | IL1R2, | 0.237344378396056, | 0.0430463438168161, | |
| | | | | | | | | IL1RL2 | 0.126970254426939 | 0.0469360657670677 | |
| CD vs. | imm_2_ | rs56151044 | <1.0E−3 | A | 94074 | −0.0625 | 103011329 | TMEM- | −0.2142 | 0.0381 | 0 |
| Ctrl | 102377761 | | | | | | | 182 | | | |
| CD vs. | imm_2_ | rs62152662 | <1.0E−3 | A | 93166 | 0.0592 | 102962341 | IL1R1, | 0.0496830463208274, | 0.0406804596047685, | 0, 0, 0 |
| Ctrl | 102328773 | | | | | | | IL1R2, | 0.237344378396056, | 0.0430463438168161, | |
| | | | | | | | | IL1RL2 | 0.126970254426939 | 0.0469360657670677 | |
| CD vs. | imm_2_ | rs17651485 | <1.0E−3 | A | 94085 | −0.0607 | 103001650 | TMEM- | −0.2142 | 0.0381 | 0 |
| Ctrl | 102368082 | | | | | | | 182 | | | |
| Crohn's | imm_2_ | rs1921622 | <1.0E−3 | G | 7967 | 1.2020 | 102966067 | MAP- | 0.0496 | 0.0167 | 0 |
| Disease | 102332499 | | | | | | | 4K4 | | | |
| vs. non- | | | | | | | | | | | |
| IBD | | | | | | | | | | | |
| Controls | | | | | | | | | | | |
| Crohn's | imm_2_ | rs12999364 | <1.0E−3 | A | 7967 | 0.8526 | 102974129 | NA | NA | NA | NA |
| Disease | 102340561 | | | | | | | | | | |
| vs. non- | | | | | | | | | | | |
| IBD | | | | | | | | | | | |
| Controls | | | | | | | | | | | |
| Crohn's | imm_2_ | rs12987977 | <1.0E−3 | C | 7967 | 0.8526 | 102975336 | NA | NA | NA | NA |
| Disease | 102341768 | | | | | | | | | | |
| vs. non- | | | | | | | | | | | |
| IBD | | | | | | | | | | | |
| Controls | | | | | | | | | | | |
| Crohn's | imm_2_ | rs2287037 | <1.0E−3 | A | 7965 | 0.8533 | 102979028 | NA | NA | NA | NA |
| Disease | 102345460 | | | | | | | | | | |
| vs. non- | | | | | | | | | | | |
| IBD | | | | | | | | | | | |
| Controls | | | | | | | | | | | |
| Crohn's | imm_2_ | rs1420101 | <1.0E−3 | A | 7966 | 0.8529 | 102957716 | NA | NA | NA | NA |
| Disease | 102324148 | | | | | | | | | | |
| vs. non- | | | | | | | | | | | |
| IBD | | | | | | | | | | | |
| Controls | | | | | | | | | | | |
| Crohn's | rs12998521 | rs12998521 | <1.0E−3 | A | 7965 | 0.8541 | 102974417 | NA | NA | NA | NA |
| Disease | | | | | | | | | | | |
| vs. non- | | | | | | | | | | | |
| IBD | | | | | | | | | | | |
| Controls | | | | | | | | | | | |
| Crohn's | imm_2_ | rs11123923 | <1.0E−3 | A | 7967 | 0.8550 | 102967844 | NA | NA | NA | NA |
| Disease | 102334276 | | | | | | | | | | |
| vs. non- | | | | | | | | | | | |
| IBD | | | | | | | | | | | |
| Controls | | | | | | | | | | | |
| Crohn's | imm_2_ | rs13001325 | <1.0E−3 | A | 7967 | 0.8551 | 102939036 | NA | NA | NA | NA |
| Disease | 102305468 | | | | | | | | | | |
| vs. non- | | | | | | | | | | | |
| IBD | | | | | | | | | | | |
| Controls | | | | | | | | | | | |
| Crohn's | imm_2_ | rs12479210 | <1.0E−3 | A | 7967 | 0.8553 | 102949161 | NA | NA | NA | NA |
| Disease | 102315593 | | | | | | | | | | |
| vs. non- | | | | | | | | | | | |
| IBD | | | | | | | | | | | |
| Controls | | | | | | | | | | | |
| Crohn's | imm_2_ | rs13020553 | <1.0E−3 | G | 7966 | 0.8556 | 102931826 | NA | NA | NA | NA |
| Disease | 102298258 | | | | | | | | | | |
| vs. non- | | | | | | | | | | | |
| IBD | | | | | | | | | | | |
| Controls | | | | | | | | | | | |
| Crohn's | imm_2_ | rs950880 | <1.0E−3 | A | 7967 | 0.8559 | 102932562 | NA | NA | NA | NA |
| Disease | 102298994 | | | | | | | | | | |
| vs. non- | | | | | | | | | | | |
| IBD | | | | | | | | | | | |
| Controls | | | | | | | | | | | |
| Crohn's | imm_2_ | rs1558627 | <1.0E−3 | G | 7956 | 1.1840 | 102984684 | NA | NA | NA | NA |
| Disease | 102351116 | | | | | | | | | | |
| vs. non- | | | | | | | | | | | |
| IBD | | | | | | | | | | | |
| Controls | | | | | | | | | | | |
| Crohn's | imm_2_ | rs3771170 | <1.0E−3 | A | 7966 | 1.1830 | 102985980 | NA | NA | NA | NA |
| Disease | 102352412 | | | | | | | | | | |
| vs. non- | | | | | | | | | | | |
| IBD | | | | | | | | | | | |
| Controls | | | | | | | | | | | |
| Crohn's | imm_2_ | rs2058622 | <1.0E−3 | A | 7967 | 1.1830 | 102985424 | NA | NA | NA | NA |
| Disease | 102351856 | | | | | | | | | | |
| vs. non- | | | | | | | | | | | |
| IBD | | | | | | | | | | | |
| Controls | | | | | | | | | | | |
| Crohn's | imm_2_ | rs13015714 | <1.0E−3 | C | 7964 | 1.1790 | 102971865 | NA | NA | NA | NA |
| Disease | 102338297 | | | | | | | | | | |
| vs. non- | | | | | | | | | | | |
| IBD | | | | | | | | | | | |
| Controls | | | | | | | | | | | |
| Crohn's | imm_2_ | rs2270297 | <1.0E−3 | A | 7967 | 1.1740 | 102992675 | NA | NA | NA | NA |
| Disease | 102359107 | | | | | | | | | | |
| vs. non- | | | | | | | | | | | |
| IBD | | | | | | | | | | | |
| Controls | | | | | | | | | | | |
| Crohn's | imm_2_ | rs6753717 | <1.0E−3 | A | 7967 | 1.1740 | 102993161 | NA | NA | NA | NA |
| Disease | 102359593 | | | | | | | | | | |
| vs. non- | | | | | | | | | | | |
| IBD | | | | | | | | | | | |
| Controls | | | | | | | | | | | |
| Crohn's | imm_2_ | rs10190555 | <1.0E−3 | A | 7967 | 1.1740 | 102994056 | NA | NA | NA | NA |
| Disease | 102360488 | | | | | | | | | | |
| vs. non- | | | | | | | | | | | |
| IBD | | | | | | | | | | | |
| Controls | | | | | | | | | | | |
| Crohn's | imm_2_ | rs6750020 | <1.0E−3 | G | 7967 | 1.1740 | 102994714 | NA | NA | NA | NA |
| Disease | 102361146 | | | | | | | | | | |
| vs. non- | | | | | | | | | | | |
| IBD | | | | | | | | | | | |
| Controls | | | | | | | | | | | |
| Crohn's | imm_2_ | rs3755274 | <1.0E−3 | A | 7967 | 1.1740 | 103002395 | NA | NA | NA | NA |
| Disease | 102368827 | | | | | | | | | | |
| vs. non- | | | | | | | | | | | |
| IBD | | | | | | | | | | | |
| Controls | | | | | | | | | | | |
| Crohn's | imm_2_ | rs2241117 | <1.0E−3 | A | 7967 | 1.1740 | 103003043 | NA | NA | NA | NA |
| Disease | 102369475 | | | | | | | | | | |
| vs. non- | | | | | | | | | | | |
| IBD | | | | | | | | | | | |
| Controls | | | | | | | | | | | |
| Crohn's | imm_2_ | rs12712145 | <1.0E−3 | A | 7965 | 1.1720 | 103008710 | NA | NA | NA | NA |
| Disease | 102375142 | | | | | | | | | | |
| vs. non- | | | | | | | | | | | |
| IBD | | | | | | | | | | | |
| Controls | | | | | | | | | | | |
| Crohn's | imm_2_ | rs3860444 | <1.0E−3 | A | 7967 | 1.1720 | 103007623 | NA | NA | NA | NA |
| Disease | 102374055 | | | | | | | | | | |
| vs. non- | | | | | | | | | | | |
| IBD | | | | | | | | | | | |
| Controls | | | | | | | | | | | |
| Crohn's | imm_2_ | rs1568681 | <1.0E−3 | G | 7967 | 1.1710 | 103014696 | NA | NA | NA | NA |
| Disease | 102381128 | | | | | | | | | | |
| vs. non- | | | | | | | | | | | |
| IBD | | | | | | | | | | | |
| Controls | | | | | | | | | | | |
| Crohn's | imm_2_ | rs11123926 | <1.0E−3 | C | 7967 | 1.1700 | 103016044 | NA | NA | NA | NA |
| Disease | 102382476 | | | | | | | | | | |
| vs. non- | | | | | | | | | | | |
| IBD | | | | | | | | | | | |
| Controls | | | | | | | | | | | |
| Crohn's | imm_2_ | rs4851007 | <1.0E−3 | A | 7965 | 1.1680 | 103024813 | NA | NA | NA | NA |
| Disease | 102391245 | | | | | | | | | | |
| vs. non- | | | | | | | | | | | |
| IBD | | | | | | | | | | | |
| Controls | | | | | | | | | | | |
| Crohn's | imm_2_ | rs1420103 | <1.0E−3 | A | 7967 | 1.1660 | 102948632 | NA | NA | NA | NA |
| Disease | 102315064 | | | | | | | | | | |
| vs. non- | | | | | | | | | | | |
| IBD | | | | | | | | | | | |
| Controls | | | | | | | | | | | |
| Crohn's | imm_2_ | rs6543116 | <1.0E−3 | A | 7967 | 1.1660 | 102927726 | NA | NA | NA | NA |
| Disease | 102294158 | | | | | | | | | | |
| vs. non- | | | | | | | | | | | |
| IBD | | | | | | | | | | | |
| Controls | | | | | | | | | | | |
| Crohn's | imm_2_ | rs1807782 | <1.0E−3 | G | 7967 | 1.1670 | 103033147 | NA | NA | NA | NA |
| Disease | 102399579 | | | | | | | | | | |
| vs. non- | | | | | | | | | | | |
| IBD | | | | | | | | | | | |
| Controls | | | | | | | | | | | |
| Crohn's | imm_2_ | rs1035127 | <1.0E−3 | A | 7962 | 1.1650 | 103019919 | NA | NA | NA | NA |
| Disease | 102386351 | | | | | | | | | | |
| vs. non- | | | | | | | | | | | |
| IBD | | | | | | | | | | | |
| Controls | | | | | | | | | | | |
| Crohn's | imm_2_ | rs17026974 | <1.0E−3 | A | 7967 | 0.8639 | 102952360 | NA | NA | NA | NA |
| Disease | 102318792 | | | | | | | | | | |
| vs. non- | | | | | | | | | | | |
| IBD | | | | | | | | | | | |
| Controls | | | | | | | | | | | |
| Crohn's | imm_2_ | rs873022 | >1.0E−3 | A | 7967 | 0.8662 | 102955683 | NA | NA | NA | NA |
| Disease | 102322115 | | | | | | | | | | |
| vs. non- | | | | | | | | | | | |
| IBD | | | | | | | | | | | |
| Controls | | | | | | | | | | | |
| Crohn's | imm_2_ | rs3732129 | >1.0E−3 | G | 7967 | 0.8662 | 102957532 | NA | NA | NA | NA |
| Disease | 102323964 | | | | | | | | | | |
| vs. non- | | | | | | | | | | | |
| IBD | | | | | | | | | | | |
| Controls | | | | | | | | | | | |

### Example 2. IL18R1 SNPs Associated with CD and Stricturing Phenotype Isolated to the Ileocolonic Region of the Intestine

Genotyping data was collected from a cohort of patients diagnosed with Crohn's disease (CD) with stricturing and CD localized at the ileocolonic region of the intestine. Genotyping was performed at Cedars-Sinai Medical Center using the Illumina Immuno-BeadChip array. Markers were excluded from analysis based on: Hardy-Weinberg Equilibrium p≤10⁻⁴; missingness in SNPs of >2%; minor allele frequency <1%. Related individuals (Pi-hat scores >0.25) were identified using identity-by-descent and excluded from analysis (PLINK). Admixture was used to generate ethnicity proportion estimations for all individuals. Only subjects identified by admixture as Caucasian (proportion <0.75) were included in the analysis. Table 2 provides SNPs associated with stricturing with evidence of penetrating and CD localized at the ileocolonic region of the intestine.

TABLE 2
IL18R1 SNPs Associated with CD and Stricturing with Evidence of Penetrating
Phenotypersolated to the Ileocolonic Region of the Intestine (L3 B2a + B2b v. B1)

| | PHENO- | | | | | | | base_pair_ | cis_ | eqtl_ | eqtl_ | |
|---|---|---|---|---|---|---|---|---|---|---|---|---|
| Population | TYPE | Marker | dbSNP | p_value | A1 | n_miss | OR_Z_B | CH2 | eGENE | beta | p | priority |
| CD | L3 | imm_2_102351116 | rs1558627 | <1.0E−3 | G | 6053 | 1.4020 | 102984684 | NA | NA | NA | NA |
| | B2a + | | | | | | | | | | | |
| | B2b vs | | | | | | | | | | | |
| | B1 | | | | | | | | | | | |
| CD | L3 | imm_2_102352412 | rs3771170 | <1.0E−3 | A | 6063 | 1.3960 | 102985980 | NA | NA | NA | NA |
| | B2a + s | | | | | | | | | | | |
| | B2b v | | | | | | | | | | | |
| | B1 | | | | | | | | | | | |
| CD | L3 | imm_2_102351856 | rs2058622 | <1.0E−3 | A | 6064 | 1.3950 | 102985424 | NA | NA | NA | NA |
| | B2a + | | | | | | | | | | | |
| | B2b vs | | | | | | | | | | | |
| | B1 | | | | | | | | | | | |
| CD | L3 | imm_2_102338297 | rs13015714 | <1.0E−3 | C | 6062 | 1.3800 | 102971865 | NA | NA | NA | NA |
| | B2a + | | | | | | | | | | | |
| | B2b vs | | | | | | | | | | | |
| | B1 | | | | | | | | | | | |
| CD | L3 | imm_2_102359107 | rs2270297 | <1.0E−3 | A | 6064 | 1.3710 | 102992675 | NA | NA | NA | NA |
| | B2a + | | | | | | | | | | | |
| | B2b vs | | | | | | | | | | | |
| | B1 | | | | | | | | | | | |
| CD | L3 | imm_2_102359593 | rs675371 7 | <1.0E−3 | A | 6064 | 1.3710 | 102993161 | NA | NA | NA | NA |
| | B2a + | | | | | | | | | | | |
| | B2b vs | | | | | | | | | | | |
| | B1 | | | | | | | | | | | |
| CD | L3 | imm_2_102360488 | rs10190555 | <1.0E−3 | A | 6064 | 1.3710 | 102994056 | NA | NA | NA | NA |
| | B2a + | | | | | | | | | | | |
| | B2b vs | | | | | | | | | | | |
| | B1 | | | | | | | | | | | |
| CD | L3 | imm_2_102361146 | rs6750020 | <1.0E−3 | G | 6064 | 1.3710 | 102994714 | NA | NA | NA | NA |
| | B2a + | | | | | | | | | | | |
| | B2b vs | | | | | | | | | | | |
| | B1 | | | | | | | | | | | |
| CD | L3 | imm_2_102368827 | rs3755274 | <1.0E−3 | A | 6064 | 1.3710 | 103002395 | NA | NA | NA | NA |
| | B2a + | | | | | | | | | | | |
| | B2b vs | | | | | | | | | | | |
| | B1 | | | | | | | | | | | |
| CD | L3 | imm_2_102369475 | rs2241117 | <1.0E−3 | A | 6064 | 1.3710 | 103003043 | NA | NA | NA | NA |
| | B2a + | | | | | | | | | | | |
| | B2b vs | | | | | | | | | | | |
| | B1 | | | | | | | | | | | |
| CD | L3 | imm_2_102381128 | rs1568681 | <1.0E−3 | G | 6064 | 1.3690 | 103014696 | NA | NA | NA | NA |
| | B2a + | | | | | | | | | | | |
| | B2b vs | | | | | | | | | | | |
| | B1 | | | | | | | | | | | |
| CD | L3 | imm_2_102375142 | rs12712145 | <1.0E−3 | A | 6062 | 1.3690 | 103008710 | NA | NA | NA | NA |
| | B2a + | | | | | | | | | | | |
| | B2b vs | | | | | | | | | | | |
| | B1 | | | | | | | | | | | |
| CD | L3 | imm_2_102374055 | rs3860444 | <1.0E−3 | A | 6064 | 1.3680 | 103007623 | NA | NA | NA | NA |
| | B2a + | | | | | | | | | | | |
| | B2b vs | | | | | | | | | | | |
| | B1 | | | | | | | | | | | |
| CD | L3 | imm_2_102382476 | rs11123926 | <1.0E−3 | C | 6064 | 1.3680 | 103016044 | NA | NA | NA | NA |
| | B2a + | | | | | | | | | | | |
| | B2b vs | | | | | | | | | | | |
| | B1 | | | | | | | | | | | |
| CD | L3 | imm_2_102399579 | rs1807782 | <1.0E−3 | G | 6064 | 1.3650 | 103033147 | NA | NA | NA | NA |
| | B2a + | | | | | | | | | | | |
| | B2b vs | | | | | | | | | | | |
| | B1 | | | | | | | | | | | |
| CD | L3 | imm_2_102391245 | rs4851007 | <1.0E−3 | A | 6063 | 1.3650 | 103024813 | NA | NA | NA | NA |
| | B2a + | | | | | | | | | | | |
| | B2b vs | | | | | | | | | | | |
| | B1 | | | | | | | | | | | |
| CD | L3 | imm_2_102386351 | rs1035127 | <1.0E−3 | A | 6061 | 1.3600 | 103019919 | NA | NA | NA | NA |
| | B2a + | | | | | | | | | | | |
| | B2b vs | | | | | | | | | | | |
| | B1 | | | | | | | | | | | |
| CD | L3 | imm_2_102294158 | rs6543116 | <1.0E−3 | A | 6064 | 1.3540 | 102927726 | NA | NA | NA | NA |
| | B2a + | | | | | | | | | | | |
| | B2b vs | | | | | | | | | | | |
| | B1 | | | | | | | | | | | |
| CD | L3 | imm_2_102315064 | rs1420103 | <1.0E−3 | A | 6064 | 1.3540 | 102948632 | NA | NA | NA | NA |
| | B2a + | | | | | | | | | | | |
| | B2b vs | | | | | | | | | | | |
| | B1 | | | | | | | | | | | |
| CD | L3 | imm_2_102332499 | rs1921622 | <1.0E−3 | A | 6064 | 0.7923 | 102966067 | MAP4K4 | 0.0496 | 0.0167 | 0 |
| | B2a + | | | | | | | | | | | |
| | B2b vs | | | | | | | | | | | |
| | B1 | | | | | | | | | | | |

### Example 3. IL18R1 SNPs Significantly Correlated with Morphological Defects of Ileal Paneth Cells

Genotyping data was collected from patients with Crohn's Disease (CD) with morphological defects of ileal Paneth cells, as determined using the classification set forth in VanDussen et al., “Genetic Variants Synthesize the Produce Paneth Cell Phenotypes That Define Subtypes of Crohn's Disease,” Gastroenterology 2014; 146:200-209. Genotyping was performed at Cedars-Sinai Medical Center using the Illumina Immuno-BeadChip array. Markers were excluded from analysis based on: Hardy-Weinberg Equilibrium p≤10⁻⁴; missingness in SNPs of >2%; minor allele frequency <1%. Related individuals (Pi-hat scores >0.25) were identified using identity-by-descent and excluded from analysis (PLINK). Admixture was used to generate ethnicity proportion estimations for all individuals. Only subjects identified by admixture as Caucasian (proportion <0.75) were included in the analysis. Results showed that SNPs at the IL18R1 locus provided in Table 3 are associated with morphological defects of ileal Paneth cells in CD subjects.

TABLE 3
IL18R1 SNPs Significantly Correlated with Defecrs of Ileal Paneth Cells

| | | | | | | n_ | OR_Z_ | base_pair_ | cis_ | eqtl_ | eqtl_ | |
|---|---|---|---|---|---|---|---|---|---|---|---|---|
| Population | PHENOTYPE | Marker | dbSNP | p_value | A1 | miss | B | CH2 | eGENE | beta | p | priority |
| CD | Paneth-D5 | imm_2_102341343 | rs76721133 | <1.0E−3 | C | 155 | 3.1900 | 102974911 | NA | NA | NA | NA |
| | phenotype | | | | | | | | | | | |

### Example 4. IL18R1 SNPs Associated with Inflammatory Bowel Disease

Multiple large-scale case-control association studies involving inflammatory bowel disease (IBD) in populations using gene-based single nucleotide polymorphism (SNP) markers were performed. The studies included subjects recruited at the Cedars-Sinai Inflammatory Bowel Disease Centers. The studies also included GWAS data derived from the International Inflammatory Bowel Diseases Genetic Consortium (IIBDGC) and cohorts from DeLange et al., “Genome-wide association study implicates immune activation of multiple integrin genes in inflammatory bowel disease,” Nature Genetics. Vol. 49, No. 2 (February 2017). Results showed that SNPs at the IL18R1 locus provided in Table 4 are significantly associated with IBD.

TABLE 4
IL18R1 SNPs Significantly Correlated with IBD

| PHENO- | | | | | | OR_ | base_pair_ | cis_ | | | prior- |
|---|---|---|---|---|---|---|---|---|---|---|---|
| TYPE | Marker | dbSNP | p_value | A1 | n_miss | Z_B | CH2 | eGENE | entl_beta | entl_p | ity |
| IBD vs. | imm_2_ | rs13001325 | <1.0E−3 | A | 128356 | −0.0989 | 102939036 | NA | NA | NA | NA |
| Ctrl | 102305468 | | | | | | | | | | |
| IBD vs. | imm_2_ | rs1420101 | <1.0E−3 | A | 128415 | −0.0988 | 102957716 | NA | NA | NA | NA |
| Ctrl | 102324148 | | | | | | | | | | |
| IBD vs. | imm_2_ | rs12479210 | <1.0E−3 | A | 128424 | −0.0983 | 102949161 | NA | NA | NA | NA |
| Ctrl | 102315593 | | | | | | | | | | |
| IBD vs. | imm_2_ | rs12987977 | <1.0E−3 | C | 128443 | −0.0963 | 102975336 | NA | NA | NA | NA |
| Ctrl | 102341768 | | | | | | | | | | |
| IBD vs. | imm_2_ | rs13020553 | <1.0E−3 | G | 128429 | −0.0972 | 102931826 | NA | NA | NA | NA |
| Ctrl | 102298258 | | | | | | | | | | |
| IBD vs. | imm_2_ | rs950880 | <1.0E−3 | A | 128433 | −0.0972 | 102932562 | NA | NA | NA | NA |
| Ctrl | 102298994 | | | | | | | | | | |
| IBD vs. | imm_2_ | rs12999364 | <1.0E−3 | A | 128422 | −0.0961 | 102974129 | NA | NA | NA | NA |
| Ctrl | 102340561 | | | | | | | | | | |
| IBD vs. | imm_2_ | rs2287037 | <1.0E−3 | A | 123133 | −0.0985 | 102979028 | NA | NA | NA | NA |
| Ctrl | 102345460 | | | | | | | | | | |
| IBD vs. | rs12998521 | rs12998521 | <1.0E−3 | A | 126705 | −0.0946 | 102974417 | NA | NA | NA | NA |
| Ctrl | | | | | | | | | | | |
| IBD vs. | imm_2_ | rs13019081 | <1.0E−3 | C | 124874 | −0.0962 | 102950822 | NA | NA | NA | NA |
| Ctrl | 102317254 | | | | | | | | | | |
| IBD vs. | imm_2_ | rs3732129 | <1.0E−3 | G | 128430 | −0.1016 | 102957532 | NA | NA | NA | NA |
| Ctrl | 1 02323964 | | | | | | | | | | |
| IBD vs. | imm_2_ | rs3771177 | <1.0E−3 | A | 128438 | −0.1016 | 102955860 | NA | NA | NA | NA |
| Ctrl | 102322292 | | | | | | | | | | |
| IBD vs. | imm_2_ | rs873022 | <1.0E−3 | A | 128438 | −0.1016 | 102955683 | NA | NA | NA | NA |
| Ctrl | 102322115 | | | | | | | | | | |
| IBD vs. | imm_2_ | rs17026974 | <1.0E−3 | A | 128409 | −0.1007 | 102952360 | NA | NA | NA | NA |
| Ctrl | 102318792 | | | | | | | | | | |
| IBD vs. | imm_2_ | rs1420102 | <1.0E−3 | G | 128385 | 0.0883 | 102948819 | NA | NA | NA | NA |
| Ctrl | 102315251 | | | | | | | | | | |
| IBD vs. | imm_2_ | rs13017455 | <1.0E−3 | A | 125514 | −0.0929 | 102964742 | NA | NA | NA | NA |
| Ctrl | 102331174 | | | | | | | | | | |
| IBD vs. | imm_2_ | rs12712141 | <1.0E−3 | A | 128443 | 0.0881 | 102953067 | NA | NA | NA | NA |
| Ctrl | 102319499 | | | | | | | | | | |
| IBD vs. | imm_2_ | rs1420088 | <1.0E−3 | A | 128434 | 0.0878 | 102939434 | NA | NA | NA | NA |
| Ctrl | 102305866 | | | | | | | | | | |
| IBD vs. | imm_2_ | rs1558619 | <1.0E−3 | C | 128433 | 0.0877 | 102931550 | NA | NA | NA | NA |
| Ctrl | 102297982 | | | | | | | | | | |
| IBD vs. | imm_2_ | rs12466380 | <1.0E−3 | A | 128438 | 0.0876 | 102948939 | NA | NA | NA | NA |
| Ctrl | 102315371 | | | | | | | | | | |
| IBD vs. | imm_2_ | rs1997467 | <1.0E−3 | A | 128423 | 0.0876 | 102951073 | NA | NA | NA | NA |
| Ctrl | 102317505 | | | | | | | | | | |
| IBD vs. | imm_2_ | rs11690443 | <1.0E−3 | T | 128429 | 0.0872 | 102936131 | NA | NA | NA | NA |
| Ctrl | 102302563 | | | | | | | | | | |
| IBD vs. | imm_2_ | rs4142132 | <1.0E−3 | G | 128437 | 0.0870 | 102937482 | NA | NA | NA | NA |
| Ctrl | 102303914 | | | | | | | | | | |
| IBD vs. | imm_2_ | rs11123923 | <1.0E−3 | A | 128409 | −0.0900 | 102967844 | NA | NA | NA | NA |
| Ctrl | 102334276 | | | | | | | | | | |
| IBD vs. | imm_2_ | rs12712135 | <1.0E−3 | A | 128436 | 0.0869 | 102930948 | NA | NA | NA | NA |
| Ctrl | 102297380 | | | | | | | | | | |
| IBD vs. | imm_2_ | rs1362350 | <1.0E−3 | G | 127309 | 0.0876 | 102951798 | NA | NA | NA | NA |
| Ctrl | 102318230 | | | | | | | | | | |
| IBD vs. | imm_2_ | rs974389 | <1.0E−3 | G | 128439 | 0.0867 | 102936981 | NA | NA | NA | NA |
| Ctrl | 102303413 | | | | | | | | | | |
| IBD vs. | imm_2_ | rs12996505 | <1.0E−3 | A | 128438 | 0.0868 | 102931802 | NA | NA | NA | NA |
| Ctrl | 102298234 | | | | | | | | | | |
| IBD vs. | imm_2_ | rs11685480 | <1.0E−3 | G | 128411 | 0.0864 | 102927086 | NA | NA | NA | NA |
| Ctrl | 102293518 | | | | | | | | | | |
| IBD vs. | imm_2_ | rs11685424 | <1.0E−3 | G | 128439 | 0.0863 | 102926981 | NA | NA | NA | NA |
| Ctrl | 102293413 | | | | | | | | | | |
| IBD vs. | imm_2_ | rs6706844 | <1.0E−3 | A | 128412 | 0.0864 | 102940412 | NA | NA | NA | NA |
| Ctrl | 102306844 | | | | | | | | | | |
| IBD vs. | imm_2_ | rs10183388 | <1.0E−3 | G | 128439 | 0.0863 | 102932247 | NA | NA | NA | NA |
| Ctrl | 102298679 | | | | | | | | | | |
| IBD vs. | imm_2_ | rs13020793 | <1.0E−3 | G | 128421 | 0.0863 | 102931926 | NA | NA | NA | NA |
| Ctrl | 102298358 | | | | | | | | | | |
| IBD vs. | imm_2_ | rs10189711 | <1.0E−3 | A | 128434 | 0.0862 | 102930881 | NA | NA | NA | NA |
| Ctrl | 1 02297313 | | | | | | | | | | |
| IBD vs. | imm_2_ | rs1558622 | <1.0E−3 | G | 128397 | 0.0859 | 102930147 | NA | NA | NA | NA |
| Ctrl | 102296579 | | | | | | | | | | |
| IBD vs. | imm_2_ | rs6733174 | <1.0E−3 | A | 128441 | 0.0862 | 102929012 | NA | NA | NA | NA |
| Ctrl | 102295444 | | | | | | | | | | |
| IBD vs. | imm_2_ | rs10189202 | <1.0E−3 | A | 128443 | 0.0856 | 102930380 | NA | NA | NA | NA |
| Ctrl | 102296812 | | | | | | | | | | |
| IBD vs. | imm_2_ | rs10191914 | <1.0E−3 | A | 128440 | 0.0856 | 102930657 | NA | NA | NA | NA |
| Ctrl | 102297089 | | | | | | | | | | |
| IBD vs. | imm_2_ | rs59247511 | <1.0E−3 | A | 122976 | 0.0876 | 102954190 | NA | NA | NA | NA |
| Ctrl | 102320622 | | | | | | | | | | |
| IBD vs. | imm_2_ | rs11123918 | <1.0E−3 | A | 127911 | 0.0857 | 102935237 | NA | NA | NA | NA |
| Ctrl | 102301669 | | | | | | | | | | |
| IBD vs. | imm_2_ | rs1420098 | <1.0E−3 | G | 120004 | −0.0906 | 102984279 | NA | NA | NA | NA |
| Ctrl | 102350711 | | | | | | | | | | |
| IBD vs. | imm_2_ | rs17027006 | <1.0E−3 | C | 128437 | −0.0967 | 102965332 | IL1RL1 | −0.0663 | 0.0409 | IL1- |
| Ctrl | 102331764 | | | | | | | | | | RL1 |
| IBD vs. | imm_2_ | rs6710885 | <1.0E−3 | G | 121235 | −0.0902 | 102977537 | NA | NA | NA | NA |
| Ctrl | 102343969 | | | | | | | | | | |
| IBD vs. | imm_2_ | rs1921622 | <1.0E−3 | G | 128426 | 0.0853 | 102966067 | MAP- | 0.0496 | 0.0167 | 0 |
| Ctrl | 102332499 | | | | | | | 4K4 | | | |
| IBD vs. | imm_2_ | rs12469506 | <1.0E−3 | A | 128441 | −0.0959 | 102965871 | IL1RL1 | −0.0659 | 0.0477 | IL1- |
| Ctrl | 102332303 | | | | | | | | | | RL1 |
| IBD vs. | imm_2_ | rs2080289 | <1.0E−3 | A | 128393 | −0.0956 | 102995020 | IL1RL1 | −0.0673 | 0.0437 | 0 |
| Ctrl | 102361452 | | | | | | | | | | |
| IBD vs. | imm_2_ | rs485157 0 | <1.0E−3 | G | 128424 | −0.0955 | 103006387 | IL1RL1 | −0.0673 | 0.0437 | 0 |
| Ctrl | 102372819 | | | | | | | | | | |
| IBD vs. | imm_2_ | rs3771171 | <1.0E−3 | G | 127351 | −0.0960 | 102985950 | NA | NA | NA | NA |
| Ctrl | 102352382 | | | | | | | | | | |
| IBD vs. | imm_2_ | rs3771172 | <1.0E−3 | A | 128434 | −0.0954 | 102985812 | NA | NA | NA | NA |
| Ctrl | 102352244 | | | | | | | | | | |
| IBD vs. | imm_2_ | rs17027087 | <1.0E−3 | A | 128442 | −0.0952 | 103015918 | IL1RL1 | −0.0673 | 0.0437 | 0 |
| Ctrl | 1 02382350 | | | | | | | | | | |
| IBD vs. | imm_2_ | rs2160202 | <1.0E−3 | A | 127368 | −0.0957 | 102986154 | NA | NA | NA | NA |
| Ctrl | 102352586 | | | | | | | | | | |
| IBD vs. | imm_2_ | rs4851006 | <1.0E−3 | A | 127366 | −0.0957 | 103024738 | NA | NA | NA | NA |
| Ctrl | 102391170 | | | | | | | | | | |
| IBD vs. | imm_2_ | rs55927292 | <1.0E−3 | A | 128397 | −0.0950 | 102964861 | IL1RL1 | −0.0700 | 0.0305 | IL1- |
| Ctrl | 102331293 | | | | | | | | | | RL1 |
| IBD vs. | imm_2_ | rs1558620 | <1.0E−3 | A | 124132 | 0.0848 | 102931395 | NA | NA | NA | NA |
| Ctrl | 102297827 | | | | | | | | | | |
| IBD vs. | imm_2_ | rs2287034 | <1.0E−3 | A | 128430 | −0.0950 | 103010588 | IL1RL1 | −0.0673 | 0.0437 | 0 |
| Ctrl | 102377020 | | | | | | | | | | |
| IBD vs. | imm_2_ | rs3821203 | <1.0E−3 | A | 128369 | −0.0950 | 102996872 | IL1RL1 | −0.0673 | 0.0437 | 0 |
| Ctrl | 102363304 | | | | | | | | | | |
| IBD vs. | imm_2_ | rs17027060 | <1.0E−3 | G | 128438 | −0.0950 | 103007567 | IL1RL1 | −0.0673 | 0.0437 | 0 |
| Ctrl | 102373999 | | | | | | | | | | |
| IBD vs. | imm_2_ | rs56258475 | <1.0E−3 | G | 128385 | −0.0949 | 102999312 | IL1RL1 | −0.0673 | 0.0437 | 0 |
| Ctrl | 102365744 | | | | | | | | | | |
| IBD vs. | imm_2_ | rs17027037 | <1.0E−3 | G | 128418 | −0.0949 | 102994884 | IL1RL1 | −0.0673 | 0.0437 | 0 |
| Ctrl | 102361316 | | | | | | | | | | |
| IBD vs. | imm_2_ | rs2270298 | <1.0E−3 | G | 128440 | −0.0947 | 102992079 | IL1RL1 | −0.0673 | 0.0437 | 0 |
| Ctrl | 102358511 | | | | | | | | | | |
| IBD vs. | imm_2_ | rs3732123 | <1.0E−3 | G | 128437 | −0.0947 | 103018077 | IL1RL1 | −0.0673 | 0.0437 | 0 |
| Ctrl | 102384509 | | | | | | | | | | |
| IBD vs. | imm_2_ | rs17027071 | <1.0E−3 | A | 128442 | −0.0943 | 103012674 | IL1RL1 | −0.0673 | 0.0437 | 0 |
| Ctrl | 102379106 | | | | | | | | | | |
| IBD vs. | imm_2_ | rs11683700 | <1.0E−3 | A | 128441 | −0.0943 | 102996805 | IL1RL1 | −0.0673 | 0.0437 | 0 |
| Ctrl | 102363237 | | | | | | | | | | |
| IBD vs. | imm_2_ | rs11465633 | <1.0E−3 | A | 127248 | −0.0940 | 102997733 | NA | NA | NA | NA |
| Ctrl | 102364165 | | | | | | | | | | |
| IBD vs. | imm_2_ | rs1362349 | <1.0E−3 | G | 121312 | 0.0842 | 102951972 | NA | NA | NA | NA |
| Ctrl | 102318404 | | | | | | | | | | |
| IBD vs. | imm_2_ | rs1968171 | <1.0E−3 | NA | NA | 0.0844 | 102933552 | NA | NA | NA | NA |
| Ctrl | 102299984 | | | | | | | | | | |
| IBD vs. | imm_2_ | rs3732126 | <1.0E−3 | C | 124130 | −0.0937 | 103013962 | IL1RL1 | −0.0673 | 0.0437 | 0 |
| Ctrl | 102380394 | | | | | | | | | | |
| IBD vs. | imm_2_ | rs1135354 | <1.0E−3 | C | 128411 | −0.0920 | 103014302 | IL1RL1 | −0.0673 | 0.0437 | 0 |
| Ctrl | 102380734 | | | | | | | | | | |
| IBD vs. | imm_2_ | rs55883125 | <1.0E−3 | A | 124115 | −0.0909 | 103024331 | IL1RL1 | −0.0673 | 0.0437 | 0 |
| Ctrl | 102390763 | | | | | | | | | | |
| IBD vs. | imm_2_ | rs67723747 | <1.0E−3 | NA | NA | −0.0921 | 102969807 | NA | NA | NA | NA |
| Ctrl | 102336239 | | | | | | | | | | |
| IBD vs. | imm_2_ | rs10190555 | <1.0E−3 | A | 127048 | 0.0904 | 102994056 | NA | NA | NA | NA |
| Ctrl | 102360488 | | | | | | | | | | |
| IBD vs. | imm_2_ | rs1035130 | <1.0E−3 | A | 121324 | −0.0884 | 103001402 | NA | NA | NA | NA |
| Ctrl | 102367834 | | | | | | | | | | |
| IBD vs. | imm_2_ | rs1035127 | <1.0E−3 | A | 128385 | 0.0885 | 103019919 | NA | NA | NA | NA |
| Ctrl | 102386351 | | | | | | | | | | |
| IBD vs. | imm_2_ | rs12712145 | <1.0E−3 | A | 128424 | 0.0874 | 103008710 | NA | NA | NA | NA |
| Ctrl | 102375142 | | | | | | | | | | |
| IBD vs. | imm_2_ | rs2241117 | <1.0E−3 | A | 128442 | 0.0866 | 103003043 | NA | NA | NA | NA |
| Ctrl | 102369475 | | | | | | | | | | |
| IBD vs. | imm_2_ | rs3755274 | <1.0E−3 | A | 128437 | 0.0864 | 103002395 | NA | NA | NA | NA |
| Ctrl | 102368827 | | | | | | | | | | |
| IBD vs. | imm_2_ | rs1568681 | <1.0E−3 | G | 128410 | 0.0864 | 103014696 | NA | NA | NA | NA |
| Ctrl | 102381128 | | | | | | | | | | |
| IBD vs. | imm_2_ | rs227029 | <1.0E−3 | A | 128426 | 0.0864 | 10299267 | NA | NA | NA | NA |
| Ctrl | 102359107 | | | | | | | | | | |
| IBD vs. | imm_2_ | rs3860444 | <1.0E−3 | A | 128415 | 0.0863 | 103007623 | NA | NA | NA | NA |
| Ctrl | 102374055 | | | | | | | | | | |
| IBD vs. | imm_2_ | rs2058622 | <1.0E−3 | A | 128439 | 0.0863 | 102985424 | NA | NA | NA | NA |
| Ctrl | 102351856 | | | | | | | | | | |
| IBD vs. | imm_2_ | rs6750020 | <1.0E−3 | G | 128434 | 0.0861 | 102994714 | NA | NA | NA | NA |
| Ctrl | 102361146 | | | | | | | | | | |
| IBD vs. | imm_2_ | rs4851007 | <1.0E−3 | A | 128428 | 0.0861 | 103024813 | NA | NA | NA | NA |
| Ctrl | 102391245 | | | | | | | | | | |
| IBD vs. | imm_2_ | rs6753717 | <1.0E−3 | A | 128424 | 0.0859 | 102993161 | NA | NA | NA | NA |
| Ctrl | 102359593 | | | | | | | | | | |
| IBD vs. | imm_2_ | rs1807782 | <1.0E−3 | G | 128384 | 0.0856 | 103033147 | NA | NA | NA | NA |
| Ctrl | 102399579 | | | | | | | | | | |
| IBD vs. | imm_2_ | rs4851575 | <1.0E−3 | G | 127285 | 0.0857 | 103025203 | NA | NA | NA | NA |
| Ctrl | 102391635 | | | | | | | | | | |
| IBD vs. | imm_2_ | rs1558627 | <1.0E−3 | G | 126175 | 0.0856 | 102984684 | NA | NA | NA | NA |
| Ctrl | 102351116 | | | | | | | | | | |
| IBD vs. | imm_2_ | rs55664618 | <1.0E−3 | A | 128443 | −0.0730 | 103016216 | IL1RL1 | −0.0559 | 0.0418 | 0 |
| Ctrl | 102382648 | | | | | | | | | | |
| IBD vs. | imm_2_ | rs13015714 | <1.0E−3 | C | 128429 | 0.0844 | 102971865 | NA | NA | NA | NA |
| Ctrl | 102338297 | | | | | | | | | | |
| IBD vs. | imm_2_ | rs2041740 | <1.0E−3 | A | 124067 | 0.0818 | 102989734 | NA | NA | NA | NA |
| Ctrl | 102356166 | | | | | | | | | | |
| IBD vs. | imm_2_ | rs1420103 | <1.0E−3 | A | 128436 | 0.0754 | 102948632 | NA | NA | NA | NA |
| Ctrl | 102315064 | | | | | | | | | | |
| IBD vs. | imm_2_ | rs6543116 | <1.0E−3 | A | 128409 | 0.0739 | 102927726 | NA | NA | NA | NA |
| Ctrl | 1 02294158 | | | | | | | | | | |
| IBD vs. | imm_2_ | rs4851005 | <1.0E−3 | A | 128361 | −0.0600 | 103011552 | NA | NA | NA | NA |
| Ctrl | 102377984 | | | | | | | | | | |
| IBD vs. | imm_2_ | rs62152661 | <1.0E−3 | G | 128435 | 0.0593 | 102959646 | IL1R2, | 0.231497338126034, | 0.0403730838316627, | 0, 0 |
| Ctrl | 102326078 | | | | | | | IL1RL2 | 0.122966178554851 | 0.0456541874537971 | |
| IBD vs. | imm_2_ | rs1420089 | <1.0E−3 | G | 128433 | 0.0592 | 102938389 | IL1R2, | 0.231497338126034, | 0.0403730838316627, | 0, 0 |
| Ctrl | 102304821 | | | | | | | IL1RL2 | 0.122966178554851 | 0.0456541874537971 | |
| IBD vs. | imm_2_ | rs17696376 | <1.0E−3 | A | 128437 | 0.0588 | 102965153 | IL1R1, | 0.0496830463208274, | 0.0406804596047685, | 0, 0, |
| Ctrl | 102331585 | | | | | | | IL1R2, | 0.237344378396056, | 0.0430463438168161, | 0 |
| | | | | | | | | IL1RL2 | 0.126970254426939 | 0.0469360657670677 | |
| IBD vs. | imm_2_ | rs12105808 | <1.0E−3 | T | 128437 | 0.0588 | 102974222 | IL1R1, | 0.0496830463208274, | 0.0406804596047685, | 0, 0, |
| Ctrl | 102340654 | | | | | | | IL1R2, | 0.237344378396056, | 0.0430463438168161, | 0 |
| | | | | | | | | IL1RL2 | 0.126970254426939 | 0.0469360657670677 | |
| IBD vs. | imm_2_ | rs1420095 | <1.0E−3 | G | 128411 | 0.0625 | 103012902 | IL1R1, | 0.0838534335200365, | 0.00372427407991006, | 0, 0, |
| Ctrl | 102379334 | | | | | | | IL18- | 0.11811725329138, | 0.0200804208351232, | 0, 0, |
| | | | | | | | | RAP, | −0.231864788573836, | 0.0239002404371004, | 0 |
| | | | | | | | | RPL31, | 0.170724364161569, | 0.0257519329018087, | |
| | | | | | | | | IL1RL2, | −0.263402645000446 | 0.0334284392287019 | |
| | | | | | | | | RPL31 | | | |
| IBD vs. | imm_2_ | rs56030066 | <1.0E−3 | C | 128443 | 0.0623 | 103003034 | IL1R1, | 0.0838534335200365, | 0.00372427407991006, | 0, 0, |
| Ctrl | 102369466 | | | | | | | IL18- | 0.11811725329138, | 0.0200804208351232, | 0, 0, |
| | | | | | | | | RAP, | −0.231864788573836, | 0.0239002404371004, | 0 |
| | | | | | | | | RPL31, | 0.170724364161569, | 0.0257519329018087, | |
| | | | | | | | | IL1RL2, | −0.263402645000446 | 0.0334284392287019 | |
| | | | | | | | | RPL31 | | | |
| IBD vs. | imm_2_ | rs78248680 | <1.0E−3 | G | 128435 | 0.0559 | 102968670 | IL1R1, | 0.0496830463208274, | 0.0406804596047685, | 0, 0, |
| Ctrl | 102335102 | | | | | | | IL1R2, | 0.237344378396056, | 0.0430463438168161, | 0 |
| | | | | | | | | IL1RL2 | 0.126970254426939 | 0.0469360657670677 | |
| IBD vs. | imm_2_ | rs62152662 | <1.0E−3 | A | 127038 | 0.0551 | 102962341 | IL1R1, | 0.0496830463208274, | 0.0406804596047685, | 0, 0, |
| Ctrl | 102328773 | | | | | | | IL1R2, | 0.237344378396056, | 0.0430463438168161, | 0 |
| | | | | | | | | IL1RL2 | 0.126970254426939 | 0.0469360657670677 | |
| IBD vs. | imm_2_ | rs62152714 | <1.0E−3 | G | 128441 | 0.0575 | 103032726 | IL1R1, | 0.0838534335200365, | 0.00372427407991006, | 0, |
| Ctrl | 102399158 | | | | | | | IL18- | 0.11811725329138 | 0.0200804208351232, | IL18- |
| | | | | | | | | RAP, | 0.231864788573836, | 0.0239002404371004, | RAP, |
| | | | | | | | | RPL31, | 0.170724364161569, | 0.0257519329018087, | 0, 0, |
| | | | | | | | | IL1RL2, | −0.263402645000446 | 0.0334284392287019 | 0 |
| | | | | | | | | RPL31 | | | |
| IBD vs. | imm_2_ | rs17027056 | <1.0E−3 | A | 128436 | −0.0678 | 103007051 | NA | NA | NA | NA |
| Ctrl | 102373483 | | | | | | | | | | |
| IBD vs. | imm_2_ | rs4988955 | <1.0E−3 | G | 128413 | 0.0302 | 102967928 | IL1RL1 | 0.0533 | 0.0425 | IL1- |
| Ctrl | 102334360 | | | | | | | | | | RL1 |
| IBD vs. | imm_2_ | rs9807962 | <1.0E−3 | G | 127307 | 0.0304 | 102971664 | NA | NA | NA | NA |
| Ctrl | 102338096 | | | | | | | | | | |
| IBD vs. | imm_2_ | rs9808453 | <1.0E−3 | A | 128328 | 0.0301 | 102971306 | IL1RL1 | 0.0533 | 0.0425 | IL1- |
| Ctrl | 1 02337738 | | | | | | | | | | RL1 |
| IBD vs. | imm_2_ | rs13424006 | <1.0E−3 | G | 128431 | 0.0301 | 102967236 | IL1RL1 | 0.0533 | 0.0425 | IL1- |
| Ctrl | 102333668 | | | | | | | | | | RL1 |
| IBD vs. | imm_2_ | rs11695627 | <1.0E−3 | G | 127909 | 0.0298 | 102970105 | IL1RL1 | 0.0533 | 0.0425 | IL1- |
| Ctrl | 102336537 | | | | | | | | | | RL1 |
| IBD vs. | imm_2_ | rs3771166 | <1.0E−3 | A | 128393 | 0.0295 | 102986222 | IL1RL1 | 0.0533 | 0.0425 | 0 |
| Ctrl | 102352654 | | | | | | | | | | |
| IBD vs. | imm_2_ | rs10173193 | <1.0E−3 | A | 128395 | 0.0293 | 102975050 | IL1RL1 | 0.0533 | 0.0425 | 0 |
| Ctrl | 102341482 | | | | | | | | | | |
| IBD vs. | imm_2_ | rs11465575 | <1.0E−3 | G | 127363 | 0.0948 | 102980976 | NA | NA | NA | NA |
| Ctrl | 102347408 | | | | | | | | | | |
| IBD vs. | imm_2_ | rs4851566 | <1.0E−3 | C | 126710 | 0.0293 | 102972799 | IL1RL1 | 0.0533 | 0.0425 | 0 |
| Ctrl | 102339231 | | | | | | | | | | |
| IBD vs. | imm_2_ | rs9308857 | <1.0E−3 | A | 128434 | 0.0290 | 102979624 | IL1RL1 | 0.0533 | 0.0425 | 0 |
| Ctrl | 102346056 | | | | | | | | | | |
| IBD vs. | imm_2_ | rs1974675 | <1.0E−3 | A | 128436 | 0.0287 | 102986375 | IL1RL1 | 0.0533 | 0.0425 | 0 |
| Ctrl | 102352807 | | | | | | | | | | |
| IBD vs. | imm_2_ | rs6751967 | <1.0E−3 | G | 128440 | 0.0284 | 102967413 | IL1RL1 | 0.0533 | 0.0425 | IL1- |
| Ctrl | 102333845 | | | | | | | | | | RL1 |
| IBD vs. | imm_2_ | rs1921622 | <1.0E−3 | G | 9366 | 1.2030 | 102966067 | MAP- | 0.0496 | 0.0167 | 0 |
| non-IBD | 102332499 | | | | | | | 4K4 | | | |
| Controls | | | | | | | | | | | |
| IBD vs. | imm_2_ | rs1420101 | <1.0E−3 | A | 9365 | 0.8460 | 102957716 | NA | NA | NA | NA |
| non-IBD | 102324148 | | | | | | | | | | |
| Controls | | | | | | | | | | | |
| IBD vs. | imm_2_ | rs12999364 | <1.0E−3 | A | 9366 | 0.8480 | 102974129 | NA | NA | NA | NA |
| non-IBD | 102340561 | | | | | | | | | | |
| Controls | | | | | | | | | | | |
| IBD vs. | imm_2_ | rs12987977 | <1.0E−3 | C | 9366 | 0.8481 | 102975336 | NA | NA | NA | NA |
| non-IBD | 102341768 | | | | | | | | | | |
| Controls | | | | | | | | | | | |
| IBD vs. | imm_2_ | rs13001325 | <1.0E−3 | A | 9366 | 0.8475 | 102939036 | NA | NA | NA | NA |
| non-IBD | 102305468 | | | | | | | | | | |
| Controls | | | | | | | | | | | |
| IBD vs. | imm_2_ | rs12479210 | <1.0E−3 | A | 9366 | 0.8477 | 102949161 | NA | NA | NA | NA |
| non-IBD | 102315593 | | | | | | | | | | |
| Controls | | | | | | | | | | | |
| IBD vs. | imm_2_ | rs2287037 | <1.0E−3 | A | 9364 | 0.8490 | 102979028 | NA | NA | NA | NA |
| non-IBD | 102345460 | | | | | | | | | | |
| Controls | | | | | | | | | | | |
| IBD vs. | imm_2_ | rs11123923 | <1.0E−3 | A | 9366 | 0.8491 | 102967844 | NA | NA | NA | NA |
| non-IBD | 1 02334276 | | | | | | | | | | |
| Controls | | | | | | | | | | | |
| IBD vs. | imm_2_ | rs13020553 | <1.0E−3 | G | 9365 | 0.8486 | 102931826 | NA | NA | NA | NA |
| non-IBD | 102298258 | | | | | | | | | | |
| Controls | | | | | | | | | | | |
| IBD vs. | imm_2_ | rs950880 | <1.0E−3 | A | 9366 | 0.8487 | 102932562 | NA | NA | NA | NA |
| non-IBD | 102298994 | | | | | | | | | | |
| Controls | | | | | | | | | | | |
| IBD vs. | rs12998521 | rs12998521 | <1.0E−3 | A | 9364 | 0.8494 | 102974417 | NA | NA | NA | NA |
| non-IBD | | | | | | | | | | | |
| Controls | | | | | | | | | | | |
| IBD vs. | imm_2_ | rs1558627 | <1.0E−3 | G | 9355 | 1.1690 | 102984684 | NA | NA | NA | NA |
| non-IBD | 102351116 | | | | | | | | | | |
| Controls | | | | | | | | | | | |
| IBD vs. | imm_2_ | rs3771170 | <1.0E−3 | A | 9365 | 1.1680 | 102985980 | NA | NA | NA | NA |
| non-IBD | 102352412 | | | | | | | | | | |
| Controls | | | | | | | | | | | |
| IBD vs. | imm_2_ | rs2058622 | <1.0E−3 | A | 9366 | 1.1680 | 102985424 | NA | NA | NA | NA |
| non-IBD | 102351856 | | | | | | | | | | |
| Controls | | | | | | | | | | | |
| IBD vs. | imm_2_ | rs13015714 | <1.0E−3 | C | 9363 | 1.1650 | 102971865 | NA | NA | NA | NA |
| non-IBD | 102338297 | | | | | | | | | | |
| Controls | | | | | | | | | | | |
| IBD vs. | imm_2_ | rs11123926 | <1.0E−3 | C | 9366 | 1.1610 | 103016044 | NA | NA | NA | NA |
| non-IBD | 102382476 | | | | | | | | | | |
| Controls | | | | | | | | | | | |
| IBD vs. | imm_2_ | rs2270297 | <1.0E−3 | A | 9366 | 1.1600 | 102992675 | NA | NA | NA | NA |
| non-IBD | 102359107 | | | | | | | | | | |
| Controls | | | | | | | | | | | |
| IBD vs. | imm_2_ | rs6753717 | <1.0E−3 | A | 9366 | 1.1600 | 102993161 | NA | NA | NA | NA |
| non-IBD | 102359593 | | | | | | | | | | |
| Controls | | | | | | | | | | | |
| IBD vs. | imm_2_ | rs10190555 | <1.0E−3 | A | 9366 | 1.1600 | 102994056 | NA | NA | NA | NA |
| non-IBD | 102360488 | | | | | | | | | | |
| Controls | | | | | | | | | | | |
| IBD vs. | imm_2_ | rs6750020 | <1.0E−3 | G | 9366 | 1.1600 | 102994714 | NA | NA | NA | NA |
| non-IBD | 102361146 | | | | | | | | | | |
| Controls | | | | | | | | | | | |
| IBD vs. | imm_2_ | rs3755274 | <1.0E−3 | A | 9366 | 1.1600 | 103002395 | NA | NA | NA | NA |
| non-IBD | 102368827 | | | | | | | | | | |
| Controls | | | | | | | | | | | |
| IBD vs. | imm_2_ | rs2241117 | <1.0E−3 | A | 9366 | 1.1600 | 103003043 | NA | NA | NA | NA |
| non-IBD | 102369475 | | | | | | | | | | |
| Controls | | | | | | | | | | | |
| IBD vs. | imm_2_ | rs17026974 | <1.0E−3 | A | 9366 | 0.8605 | 102952360 | NA | NA | NA | NA |
| non-IBD | 102318792 | | | | | | | | | | |
| Controls | | | | | | | | | | | |
| IBD vs. | imm_2_ | rs12712145 | <1.0E−3 | A | 9364 | 1.1600 | 103008710 | NA | NA | NA | NA |
| non-IBD | 102375142 | | | | | | | | | | |
| Controls | | | | | | | | | | | |
| IBD vs. | imm_2_ | rs3860444 | <1.0E−3 | A | 9366 | 1.1600 | 103007623 | NA | NA | NA | NA |
| non-IBD | 102374055 | | | | | | | | | | |
| Controls | | | | | | | | | | | |
| IBD vs. | imm_2_ | rs1568681 | <1.0E−3 | G | 9366 | 1.1600 | 103014696 | NA | NA | NA | NA |
| non-IBD | 102381128 | | | | | | | | | | |
| Controls | | | | | | | | | | | |
| IBD vs. | imm_2_ | rs4851007 | <1.0E−3 | A | 9364 | 1.1580 | 103024813 | NA | NA | NA | NA |
| non-IBD | 102391245 | | | | | | | | | | |
| Controls | | | | | | | | | | | |
| IBD vs. | imm_2_ | rs1807782 | <1.0E−3 | G | 9366 | 1.1580 | 103033147 | NA | NA | NA | NA |
| non-IBD | 102399579 | | | | | | | | | | |
| Controls | | | | | | | | | | | |
| IBD vs. | imm_2_ | rs3771177 | <1.0E−3 | A | 9364 | 0.8621 | 102955860 | NA | NA | NA | NA |
| non-IBD | 102322292 | | | | | | | | | | |
| Controls | | | | | | | | | | | |
| IBD vs. | imm_2_ | rs873022 | <1.0E−3 | A | 9366 | 0.8622 | 102955683 | NA | NA | NA | NA |
| non-IBD | 102322115 | | | | | | | | | | |
| Controls | | | | | | | | | | | |
| IBD vs. | imm_2_ | rs3732129 | <1.0E−3 | G | 9366 | 0.8622 | 102957532 | NA | NA | NA | NA |
| non-IBD | 102323964 | | | | | | | | | | |
| Controls | | | | | | | | | | | |
| IBD vs. | imm_2_ | rs1035127 | <1.0E−3 | A | 9361 | 1.1580 | 103019919 | NA | NA | NA | NA |
| non-IBD | 102386351 | | | | | | | | | | |
| Controls | | | | | | | | | | | |
| IBD vs. | imm_2_ | rs6543116 | <1.0E−3 | A | 9366 | 1.1450 | 102927726 | NA | NA | NA | NA |
| non-IBD | 102294158 | | | | | | | | | | |
| Controls | | | | | | | | | | | |
| IBD vs. | imm_2_ | rs1420103 | <1.0E−3 | A | 9366 | 1.1450 | 102948632 | NA | NA | NA | NA |
| non-IBD | 102315064 | | | | | | | | | | |
| Controls | | | | | | | | | | | |
| IBD vs. | imm_2_ | rs3771162 | <1.0E−3 | A | 9364 | 0.8713 | 102997174 | IL1RL1 | −0.0673 | 0.0437 | 0 |
| non-IBD | 102363606 | | | | | | | | | | |
| Controls | | | | | | | | | | | |
| IBD vs. | imm_2_ | rs17027087 | <1.0E−3 | A | 9366 | 0.8711 | 103015918 | IL1RL1 | −0.0673 | 0.0437 | 0 |
| non-IBD | 102382350 | | | | | | | | | | |
| Controls | | | | | | | | | | | |
| IBD vs. | imm_2_ | rs56386507 | <1.0E−3 | A | 9366 | 0.8721 | 102971165 | IL1RL1 | −0.0675 | 0.0376 | IL1- |
| non-IBD | 1 02337597 | | | | | | | | | | RL1 |
| Controls | | | | | | | | | | | |
| IBD vs. | imm_2_ | rs2080289 | <1.0E−3 | A | 9364 | 0.8722 | 102995020 | IL1RL1 | −0.0673 | 0.0437 | 0 |
| non-IBD | 102361452 | | | | | | | | | | |
| Controls | | | | | | | | | | | |
| IBD vs. | imm_2_ | rs3771172 | <1.0E−3 | A | 9366 | 0.8724 | 102985812 | NA | NA | NA | NA |
| non-IBD | 102352244 | | | | | | | | | | |
| Controls | | | | | | | | | | | |
| IBD vs. | imm_2_ | rs2270298 | <1.0E−3 | G | 9366 | 0.8727 | 102992079 | IL1RL1 | −0.0673 | 0.0437 | 0 |
| non-IBD | 102358511 | | | | | | | | | | |
| Controls | | | | | | | | | | | |
| IBD vs. | imm_2_ | rs3821203 | <1.0E−3 | A | 9366 | 0.8727 | 102996872 | IL1RL1 | −0.0673 | 0.0437 | 0 |
| non-IBD | 102363304 | | | | | | | | | | |
| Controls | | | | | | | | | | | |
| IBD vs. | imm_2_ | rs56258475 | <1.0E−3 | G | 9366 | 0.8727 | 102999312 | IL1RL1 | −0.0673 | 0.0437 | 0 |
| non-IBD | 102365744 | | | | | | | | | | |
| Controls | | | | | | | | | | | |
| IBD vs. | imm_2_ | rs4851570 | <1.0E−3 | G | 9366 | 0.8727 | 103006387 | IL1RL1 | −0.0673 | 0.0437 | 0 |
| non-IBD | 102372819 | | | | | | | | | | |
| Controls | | | | | | | | | | | |
| IBD vs. | imm_2_ | rs17027060 | <1.0E−3 | G | 9366 | 0.8727 | 103007567 | IL1RL1 | −0.0673 | 0.0437 | 0 |
| non-IBD | 102373999 | | | | | | | | | | |
| Controls | | | | | | | | | | | |
| IBD vs. | imm_2_ | rs3732123 | <1.0E−3 | G | 9366 | 0.8727 | 103018077 | IL1RL1 | −0.0673 | 0.0437 | 0 |
| non-IBD | 1 02384509 | | | | | | | | | | |
| Controls | | | | | | | | | | | |
| IBD vs. | imm_2_ | rs17027006 | <1.0E−3 | C | 9366 | 0.8729 | 102965332 | IL1RL1 | −0.0663 | 0.0409 | IL1- |
| non-IBD | 102331764 | | | | | | | | | | RL1 |
| Controls | | | | | | | | | | | |
| IBD vs. | imm_2_ | rs12712141 | <1.0E−3 | A | 9366 | 1.1240 | 102953067 | NA | NA | NA | NA |
| non-IBD | 1 02319499 | | | | | | | | | | |
| Controls | | | | | | | | | | | |
| IBD vs. | imm_2_ | rs2287034 | <1.0E−3 | A | 9365 | 0.8732 | 103010588 | IL1RL1 | −0.0673 | 0.0437 | 0 |
| non-IBD | 102377020 | | | | | | | | | | |
| Controls | | | | | | | | | | | |
| IBD vs. | imm_2_ | rs17027037 | <1.0E−3 | G | 9366 | 0.8735 | 102994884 | IL1RL1 | −0.0673 | 0.0437 | 0 |
| non-IBD | 102361316 | | | | | | | | | | |
| Controls | | | | | | | | | | | |
| IBD vs. | imm_2_ | rs1135354 | <1.0E−3 | C | 9362 | 0.8737 | 103014302 | IL1RL1 | −0.0673 | 0.0437 | 0 |
| non-IBD | 102380734 | | | | | | | | | | |
| Controls | | | | | | | | | | | |
| IBD vs. | imm_2_ | rs12469506 | <1.0E−3 | A | 9366 | 0.8737 | 102965871 | IL1RL1 | −0.0659 | 0.0477 | IL1- |
| non-IBD | 102332303 | | | | | | | | | | RL1 |
| Controls | | | | | | | | | | | |
| IBD vs. | imm_2_ | rs11683700 | <1.0E−3 | A | 9366 | 0.8743 | 102996805 | IL1RL1 | −0.0673 | 0.0437 | 0 |
| non-IBD | 102363237 | | | | | | | | | | |
| Controls | | | | | | | | | | | |
| IBD vs. | imm_2_ | rs55927292 | <1.0E−3 | A | 9365 | 0.8776 | 102964861 | IL1RL1 | −0.0700 | 0.0305 | IL1- |
| non-IBD | 102331293 | | | | | | | | | | RL1 |
| Controls | | | | | | | | | | | |
| IBD vs. | imm_2_ | rs17027071 | <1.0E−3 | A | 9366 | 0.8777 | 103012674 | IL1RL1 | −0.0673 | 0.0437 | 0 |
| non-IBD | 102379106 | | | | | | | | | | |
| Controls | | | | | | | | | | | |
| IBD vs. | imm_2_ | rs1997467 | <1.0E−3 | A | 9361 | 1.1150 | 102951073 | NA | NA | NA | NA |
| non-IBD | 102317505 | | | | | | | | | | |
| Controls | | | | | | | | | | | |
| IBD vs. | imm_2_ | rs1997466 | <1.0E−3 | G | 9365 | 1.1140 | 102951467 | NA | NA | NA | NA |
| non-IBD | 102317899 | | | | | | | | | | |
| Controls | | | | | | | | | | | |
| IBD vs. | imm_2_ | rs12712140 | <1.0E−3 | C | 9366 | 1.1130 | 102951062 | NA | NA | NA | NA |
| non-IBD | 102317494 | | | | | | | | | | |
| Controls | | | | | | | | | | | |

### Example 5. IL18R1 SNPs Associated with Ulcerative Colitis

Multiple large-scale case-control association studies involving ulcerative colitis (UC) in populations using gene-based single nucleotide polymorphism (SNP) markers were performed. The studies included subjects recruited at the Cedars-Sinai Inflammatory Bowel Disease Centers. The studies also included GWAS data derived from the International Inflammatory Bowel Diseases Genetic Consortium (IIBDGC) and cohorts from DeLange et al., “Genome-wide association study implicates immune activation of multiple integrin genes in inflammatory bowel disease,” Nature Genetics. Vol. 49, No. 2 (February 2017). Results showed that SNPs at the IL18R1 locus provided in Table 5 are significantly associated with UC.

TABLE 5
IL18R1 SNPs Associated with UC

| | | | | | | OR_Z_ | base_pair_ | cis_ | | | |
|---|---|---|---|---|---|---|---|---|---|---|---|
| PHENOTYPE | Marker | dbSNP | p_value | A1 | n_miss | B | CH2 | eGENE | entl_beta | entl_p | priority |
| UC vs. Ctrl | imm_2_102341768 | rs12987977 | <1.0E−3 | C | 95393 | −0.0773 | 102975336 | NA | NA | NA | NA |
| UC vs. Ctrl | imm_2_102340561 | rs12999364 | <1.0E−3 | A | 95381 | −0.0768 | 102974129 | NA | NA | NA | NA |
| UC vs. Ctrl | imm_2_102345460 | rs2287037 | <1.0E−3 | A | 92068 | −0.0786 | 102979028 | NA | NA | NA | NA |
| UC vs. Ctrl | rs12998521 | rs12998521 | <1.0E−3 | A | 94754 | −0.0764 | 102974417 | NA | NA | NA | NA |
| UC vs. Ctrl | imm_2_102334276 | rs11123923 | <1.0E−3 | A | 95379 | −0.0719 | 102967844 | NA | NA | NA | NA |
| UC vs. Ctrl | imm_2_102350711 | rs1420098 | <1.0E−3 | G | 88812 | −0.0730 | 102984279 | NA | NA | NA | NA |
| UC vs. Ctrl | imm_2_102331174 | rs13017455 | <1.0E−3 | A | 93635 | −0.0724 | 102964742 | NA | NA | NA | NA |
| UC vs. Ctrl | imm_2_102343969 | rs6710885 | <1.0E−3 | G | 89377 | −0.0726 | 102977537 | NA | NA | NA | NA |
| UC vs. Ctrl | imm_2_102305468 | rs13001325 | <1.0E−3 | A | 95338 | −0.0713 | 102939036 | NA | NA | NA | NA |
| UC vs. Ctrl | imm_2_102315593 | rs12479210 | <1.0E−3 | A | 95383 | −0.0710 | 102949161 | NA | NA | NA | NA |
| UC vs. Ctrl | imm_2_102324148 | rs1420101 | <1.0E−3 | A | 95374 | −0.0706 | 102957716 | NA | NA | NA | NA |
| UC vs. Ctrl | imm_2_102298258 | rs13020553 | <1.0E−3 | G | 95381 | −0.0703 | 102931826 | NA | NA | NA | NA |
| UC vs. Ctrl | imm_2_102298994 | rs950880 | <1.0E−3 | A | 95388 | −0.0703 | 102932562 | NA | NA | NA | NA |
| UC vs. Ctrl | imm_2_102323964 | rs3732129 | <1.0E−3 | G | 95381 | −0.0753 | 102957532 | NA | NA | NA | NA |
| UC vs. Ctrl | imm_2_102322292 | rs3771177 | <1.0E−3 | A | 95389 | −0.0752 | 102955860 | NA | NA | NA | NA |
| UC vs. Ctrl | imm_2_102322115 | rs873022 | <1.0E−3 | A | 95390 | −0.0751 | 102955683 | NA | NA | NA | NA |
| UC vs. Ctrl | imm_2_102317254 | rs13019081 | <1.0E−3 | C | 93843 | −0.0692 | 102950822 | NA | NA | NA | NA |
| UC vs. Ctrl | imm_2_102352382 | rs3771171 | <1.0E−3 | G | 94548 | −0.0748 | 102985950 | NA | NA | NA | NA |
| UC vs. Ctrl | imm_2_102318792 | rs17026974 | <1.0E−3 | A | 95369 | −0.0738 | 102952360 | NA | NA | NA | NA |
| UC vs. Ctrl | imm_2_102331764 | rs17027006 | <1.0E−3 | C | 95391 | −0.0740 | 102965332 | IL1RL1 | −0.06639 | 0.0401 | IL1RL1 |
| UC vs. Ctrl | imm_2_102352586 | rs2160202 | <1.0E−3 | A | 94558 | −0.0742 | 102986154 | NA | NA | NA | NA |
| UC vs. Ctrl | imm_2_102352244 | rs3771172 | <1.0E−3 | A | 95387 | −0.0739 | 102985812 | NA | NA | NA | NA |
| UC vs. Ctrl | imm_2_102332303 | rs12469506 | <1.0E−3 | A | 95392 | −0.0737 | 102965871 | IL1RL1 | −0.06597 | 0.0471 | IL1RL1 |
| UC vs. Ctrl | imm_2_102315251 | rs1420102 | <1.0E−3 | G | 95356 | 0.0643 | 102948819 | NA | NA | NA | NA |
| UC vs. Ctrl | imm_2_102331293 | rs55927292 | <1.0E−3 | A | 95358 | −0.0727 | 102964861 | IL1RL1 | −0.07005 | 0.0301 | IL1RL1 |
| UC vs. Ctrl | imm_2_102297380 | rs12712135 | <1.0E−3 | A | 95390 | 0.0638 | 102930948 | NA | NA | NA | NA |
| UC vs. Ctrl | imm_2_102305866 | rs1420088 | <1.0E−3 | A | 95388 | 0.0637 | 102939434 | NA | NA | NA | NA |
| UC vs. Ctrl | imm_2_102298234 | rs12996505 | <1.0E−3 | A | 95390 | 0.0635 | 102931802 | NA | NA | NA | NA |
| UC vs. Ctrl | imm_2_102297982 | rs1558619 | <1.0E−3 | C | 95387 | 0.0635 | 102931550 | NA | NA | NA | NA |
| UC vs. Ctrl | imm_2_102317505 | rs1997467 | <1.0E−3 | A | 95378 | 0.0634 | 102951073 | NA | NA | NA | NA |
| UC vs. Ctrl | imm_2_102303914 | rs4142132 | <1.0E−3 | G | 95388 | 0.0632 | 102937482 | NA | NA | NA | NA |
| UC vs. Ctrl | imm_2_102315371 | rs12466380 | <1.0E−3 | A | 95390 | 0.0631 | 102948939 | NA | NA | NA | NA |
| UC vs. Ctrl | imm_2_102361452 | rs2080289 | <1.0E−3 | A | 95372 | −0.0716 | 102995020 | IL1RL1 | −0.0673 | 0.0437 | 0 |
| UC vs. Ctrl | imm_2_102298679 | rs10183388 | <1.0E−3 | G | 95390 | 0.0630 | 102932247 | NA | NA | NA | NA |
| UC vs. Ctrl | imm_2_102302563 | rs11690443 | <1.0E−3 | T | 95389 | 0.0629 | 102936131 | NA | NA | NA | NA |
| UC vs. Ctrl | imm_2_102361316 | rs17027037 | <1.0E−3 | G | 95375 | −0.0714 | 102994884 | IL1RL1 | −0.0673 | 0.0437 | 0 |
| UC vs. Ctrl | imm_2_102372819 | rs4851570 | <1.0E−3 | G | 95382 | −0.0713 | 103006387 | IL1RL1 | −0.0673 | 0.0437 | 0 |
| UC vs. Ctrl | imm_2_102306844 | rs6706844 | <1.0E−3 | A | 95369 | 0.0628 | 102940412 | NA | NA | NA | NA |
| UC vs. Ctrl | imm_2_102293518 | rs11685480 | <1.0E−3 | G | 95373 | 0.0628 | 102927086 | NA | NA | NA | NA |
| UC vs. Ctrl | imm_2_102336239 | rs67723747 | <1.0E−3 | NA | NA | −0.0730 | 102969807 | NA | NA | NA | NA |
| UC vs. Ctrl | imm_2_102303413 | rs974389 | <1.0E−3 | G | 95390 | 0.0627 | 102936981 | NA | NA | NA | NA |
| UC vs. Ctrl | imm_2_102319499 | rs12712141 | <1.0E−3 | A | 95393 | 0.0627 | 102953067 | NA | NA | NA | NA |
| UC vs. Ctrl | imm_2_102301669 | rs11123918 | <1.0E−3 | A | 95145 | 0.0628 | 102935237 | NA | NA | NA | NA |
| UC vs. Ctrl | imm_2_102365744 | rs56258475 | <1.0E−3 | G | 95358 | −0.0710 | 102999312 | IL1RL1 | −0.0673 | 0.0437 | 0 |
| UC vs. Ctrl | imm_2_102320622 | rs59247511 | <1.0E−3 | A | 90228 | 0.0634 | 102954190 | NA | NA | NA | NA |
| UC vs. Ctrl | imm_2_102363304 | rs3821203 | <1.0E−3 | A | 95344 | −0.0710 | 102996872 | IL1RL1 | −0.0673 | 0.0437 | 0 |
| UC vs. Ctrl | imm_2_102391170 | rs4851006 | <1.0E−3 | A | 94559 | −0.0712 | 103024738 | NA | NA | NA | NA |
| UC vs. Ctrl | imm_2_102379106 | rs17027071 | <1.0E−3 | A | 95393 | −0.0708 | 103012674 | IL1RL1 | −0.0673 | 0.0437 | 0 |
| UC vs. Ctrl | imm_2_102295444 | rs6733174 | <1.0E−3 | A | 95392 | 0.0624 | 102929012 | NA | NA | NA | NA |
| UC vs. Ctrl | imm_2_102377020 | rs2287034 | <1.0E−3 | A | 95383 | −0.0708 | 103010588 | IL1RL1 | −0.0673 | 0.0437 | 0 |
| UC vs. Ctrl | imm_2_102358511 | rs2270298 | <1.0E−3 | G | 95390 | −0.0708 | 102992079 | IL1RL1 | −0.0673 | 0.0437 | 0 |
| UC vs. Ctrl | imm_2_102382350 | rs17027087 | <1.0E−3 | A | 95392 | −0.0707 | 103015918 | IL1RL1 | −0.0673 | 0.0437 | 0 |
| UC vs. Ctrl | imm_2_102298358 | rs13020793 | <1.0E−3 | G | 95378 | 0.0622 | 102931926 | NA | NA | NA | NA |
| UC vs. Ctrl | imm_2_102380394 | rs3732126 | <1.0E−3 | C | 91115 | −0.0712 | 103013962 | IL1RL1 | −0.0673 | 0.0437 | 0 |
| UC vs. Ctrl | imm_2_102318230 | rs1362350 | <1.0E−3 | G | 94516 | 0.0624 | 102951798 | NA | NA | NA | NA |
| UC vs. Ctrl | imm_2_102296812 | rs10189202 | <1.0E−3 | A | 95393 | 0.0621 | 102930380 | NA | NA | NA | NA |
| UC vs. Ctrl | imm_2_102373999 | rs17027060 | <1.0E−3 | G | 95390 | −0.0704 | 103007567 | IL1RL1 | −0.0673 | 0.0437 | 0 |
| UC vs. Ctrl | imm_2_102363237 | rs11683700 | <1.0E−3 | A | 95391 | −0.0704 | 102996805 | IL1RL1 | −0.0673 | 0.0437 | 0 |
| UC vs. Ctrl | imm_2_102297089 | rs10191914 | <1.0E−3 | A | 95392 | 0.0620 | 102930657 | NA | NA | NA | NA |
| UC vs. Ctrl | imm_2_102293413 | rs11685424 | <1.0E−3 | G | 95391 | 0.0621 | 102926981 | NA | NA | NA | NA |
| UC vs. Ctrl | imm_2_102297313 | rs10189711 | <1.0E−3 | A | 95387 | 0.0619 | 102930881 | NA | NA | NA | NA |
| UC vs. Ctrl | imm_2_102296579 | rs1558622 | <1.0E−3 | G | 95364 | 0.0619 | 102930147 | NA | NA | NA | NA |
| UC vs. Ctrl | imm_2_102384509 | rs3732123 | <1.0E−3 | G | 95389 | −0.0700 | 103018077 | IL1RL1 | −0.0673 | 0.0437 | 0 |
| UC vs. Ctrl | imm_2_102364165 | rs11465633 | <1.0E−3 | A | 94483 | −0.0702 | 102997733 | NA | NA | NA | NA |
| UC vs. Ctrl | imm_2_102380734 | rs1135354 | <1.0E−3 | C | 95377 | −0.0682 | 103014302 | IL1RL1 | −0.0673 | 0.0437 | 0 |
| UC vs. Ctrl | imm_2_102297827 | rs1558620 | <1.0E−3 | A | 91113 | 0.0600 | 102931395 | NA | NA | NA | NA |
| UC vs. Ctrl | imm_2_102332499 | rs1921622 | <1.0E−3 | G | 95383 | 0.0599 | 102966067 | MAP4K4 | 0.0496 | 0.0167 | 0 |
| UC vs. Ctrl | imm_2_102318404 | rs1362349 | <1.0E−3 | G | 89644 | 0.0604 | 102951972 | NA | NA | NA | NA |
| UC vs. Ctrl | imm_2_102390763 | rs55883125 | <1.0E−3 | A | 91103 | −0.0671 | 103024331 | IL1RL1 | −0.0673 | 0.0437 | 0 |
| UC vs. Ctrl | imm_2_102367834 | rs1035130 | <1.0E−3 | A | 90081 | −0.0662 | 103001402 | NA | NA | NA | NA |
| UC vs. Ctrl | imm_2_102382648 | rs55664618 | <1.0E−3 | A | 95393 | −0.0578 | 103016216 | IL1RL1 | −0.0559 | 0.0418 | 0 |
| UC vs. Ctrl | imm_2_102299984 | rs1968171 | <1.0E−3 | NA | NA | 0.0557 | 102933552 | NA | NA | NA | NA |
| UC vs. Ctrl | imm_2_102360488 | rs10190555 | <1.0E−3 | A | 94433 | 0.0590 | 102994056 | NA | NA | NA | NA |
| UC vs. Ctrl | imm_2_102386351 | rs1035127 | <1.0E−3 | A | 95362 | 0.0542 | 103019919 | NA | NA | NA | NA |
| UC vs. Ctrl | imm_2_102375142 | rs12712145 | <1.0E−3 | A | 95381 | 0.0540 | 103008710 | NA | NA | NA | NA |
| UC vs. Ctrl | imm_2_102381128 | rs1568681 | <1.0E−3 | G | 95371 | 0.0531 | 103014696 | NA | NA | NA | NA |
| UC vs. Ctrl | imm_2_102351856 | rs2058622 | <1.0E−3 | A | 95389 | 0.0531 | 102985424 | NA | NA | NA | NA |
| UC vs. Ctrl | imm_2_102368827 | rs3755274 | <1.0E−3 | A | 95390 | 0.0529 | 103002395 | NA | NA | NA | NA |
| UC vs. Ctrl | imm_2_102361146 | rs6750020 | <1.0E−3 | G | 95385 | 0.0528 | 102994714 | NA | NA | NA | NA |
| UC vs. Ctrl | imm_2_102369475 | rs2241117 | <1.0E−3 | A | 95392 | 0.0528 | 103003043 | NA | NA | NA | NA |
| UC vs. Ctrl | imm_2_102359593 | rs6753717 | <1.0E−3 | A | 95379 | 0.0528 | 102993161 | NA | NA | NA | NA |
| UC vs. Ctrl | imm_2_102359107 | rs2270297 | <1.0E−3 | A | 95381 | 0.0527 | 102992675 | NA | NA | NA | NA |
| UC vs. Ctrl | imm_2_102374055 | rs3860444 | <1.0E−3 | A | 95375 | 0.0527 | 103007623 | NA | NA | NA | NA |
| UC vs. Ctrl | imm_2_102351116 | rs1558627 | <1.0E−3 | G | 94316 | 0.0527 | 102984684 | NA | NA | NA | NA |
| UC vs. Ctrl | imm_2_102399579 | rs1807782 | <1.0E−3 | G | 95356 | 0.0523 | 103033147 | NA | NA | NA | NA |
| UC vs. Ctrl | imm_2_102391245 | rs4851007 | <1.0E−3 | A | 95381 | 0.0516 | 103024813 | NA | NA | NA | NA |
| UC vs. Ctrl | imm_2_102338297 | rs13015714 | <1.0E−3 | C | 95382 | 0.0512 | 102971865 | NA | NA | NA | NA |
| UC vs. Ctrl | imm_2_102391635 | rs4851575 | <1.0E−3 | G | 94510 | 0.0503 | 103025203 | NA | NA | NA | NA |
| UC vs. Ctrl | imm_2_102356166 | rs2041740 | <1.0E−3 | A | 91089 | 0.0496 | 102989734 | NA | NA | NA | NA |
| UC vs. Ctrl | imm_2_102377984 | rs4851005 | <1.0E−3 | A | 95340 | −0.0409 | 103011552 | NA | NA | NA | NA |
| UC vs. Ctrl | imm_2_102338096 | rs9807962 | <1.0E−3 | G | 94529 | 0.0380 | 102971664 | NA | NA | NA | NA |
| UC vs. Ctrl | imm_2_102341482 | rs10173193 | <1.0E−3 | A | 95368 | 0.0372 | 102975050 | IL1RL1 | 0.0533 | 0.0425 | 0 |
| UC vs. Ctrl | imm_2_102334360 | rs4988955 | <1.0E−3 | G | 95373 | 0.0371 | 102967928 | IL1RL1 | 0.0533 | 0.0425 | IL1RL1 |
| UC vs. Ctrl | imm_2_102346056 | rs9308857 | <1.0E−3 | A | 95388 | 0.0371 | 102979624 | IL1RL1 | 0.0533 | 0.0425 | 0 |
| UC vs. Ctrl | imm_2_102337738 | rs9808453 | <1.0E−3 | A | 95325 | 0.0370 | 102971306 | IL1RL1 | 0.0533 | 0.0425 | IL1RL1 |
| UC vs. Ctrl | imm_2_102339231 | rs4851566 | <1.0E−3 | C | 94760 | 0.0372 | 102972799 | IL1RL1 | 0.0533 | 0.0425 | 0 |
| UC vs. Ctrl | imm_2_102333668 | rs13424006 | <1.0E−3 | G | 95385 | 0.0369 | 102967236 | IL1RL1 | 0.0533 | 0.0425 | IL1RL1 |
| UC vs. Ctrl | imm_2_102352654 | rs3771166 | <1.0E−3 | A | 95357 | 0.0369 | 102986222 | IL1RL1 | 0.0533 | 0.0425 | 0 |
| UC vs. Ctrl | imm_2_102294158 | rs6543116 | <1.0E−3 | A | 95366 | 0.0411 | 102927726 | NA | NA | NA | NA |
| UC vs. Ctrl | imm_2_102351056 | rs1362348 | <1.0E−3 | G | 90305 | 0.0369 | 102984624 | NA | NA | NA | NA |
| UC vs. Ctrl | imm_2_102315064 | rs1420103 | <1.0E−3 | A | 95390 | 0.0411 | 102948632 | NA | NA | NA | NA |
| UC vs. Ctrl | imm_2_102333845 | rs6751967 | <1.0E−3 | G | 95391 | 0.0361 | 102967413 | IL1RL1 | 0.0533 | 0.0425 | IL1RL1 |
| Ulcerative | imm_2_102332499 | rs1921622 | <1.0E−3 | G | 6864 | 1.1940 | 102966067 | MAP4K4 | 0.0496 | 0.0167 | 0 |
| Colitis vs. | | | | | | | | | | | |
| non-IBD | | | | | | | | | | | |
| Controls | | | | | | | | | | | |
| Ulcerative | imm_2_102334276 | rs11123923 | <1.0E−3 | A | 6864 | 0.8381 | 102967844 | NA | NA | NA | NA |
| Colitis vs. | | | | | | | | | | | |
| non-IBD | | | | | | | | | | | |
| Controls | | | | | | | | | | | |
| Ulcerative | imm_2_102340561 | rs12999364 | <1.0E−3 | A | 6864 | 0.8396 | 102974129 | NA | NA | NA | NA |
| Colitis vs. | | | | | | | | | | | |
| non-IBD | | | | | | | | | | | |
| Controls | | | | | | | | | | | |
| Ulcerative | imm_2_102341768 | rs12987977 | <1.0E−3 | C | 6864 | 0.8399 | 102975336 | NA | NA | NA | NA |
| Colitis vs. | | | | | | | | | | | |
| non-IBD | | | | | | | | | | | |
| Controls | | | | | | | | | | | |
| Ulcerative | rs12998521 | rs12998521 | <1.0E−3 | A | 6863 | 0.8403 | 102974417 | NA | NA | NA | NA |
| Colitis vs. | | | | | | | | | | | |
| non-IBD | | | | | | | | | | | |
| Controls | | | | | | | | | | | |
| Ulcerative | imm_2_102345460 | rs2287037 | <1.0E−3 | A | 6862 | 0.8408 | 102979028 | NA | NA | NA | NA |
| Colitis vs. | | | | | | | | | | | |
| non-IBD | | | | | | | | | | | |
| Controls | | | | | | | | | | | |
| Ulcerative | imm_2_102305468 | rs13001325 | <1.0E−3 | A | 6864 | 0.8415 | 102939036 | NA | NA | NA | NA |
| Colitis vs. | | | | | | | | | | | |
| non-IBD | | | | | | | | | | | |
| Controls | | | | | | | | | | | |
| Ulcerative | imm_2_102315593 | rs12479210 | <1.0E−3 | A | 6864 | 0.8417 | 102949161 | NA | NA | NA | NA |
| Colitis vs. | | | | | | | | | | | |
| non-IBD | | | | | | | | | | | |
| Controls | | | | | | | | | | | |
| Ulcerative | imm_2_102298258 | rs13020535 | <1.0E−3 | G | 6864 | 0.8433 | 102931826 | NA | NA | NA | NA |
| Colitis vs. | | | | | | | | | | | |
| non-IBD | | | | | | | | | | | |
| Controls | | | | | | | | | | | |
| Ulcerative | imm_2_102298994 | rs950880 | <1.0E−3 | A | 6864 | 0.8433 | 102932562 | NA | NA | NA | NA |
| Colitis vs. | | | | | | | | | | | |
| non-IBD | | | | | | | | | | | |
| Controls | | | | | | | | | | | |
| Ulcerative | imm_2_102324148 | rs1420101 | <1.0E−3 | A | 6863 | 0.8442 | 102957716 | NA | NA | NA | NA |
| Colitis vs. | | | | | | | | | | | |
| non-IBD | | | | | | | | | | | |
| Controls | | | | | | | | | | | |

TABLE 6
Description of Abbreviations Used in Disclosed Tables

| Term | Description |
|---|---|
| dbSNP | dbSNP 147 rsID for SNP associated with phenotype |
| A1 | Minor allele for SNP |
| Study Size | Number of subjects in cohort |
| (n_miss) | |
| Beta (OR_Z_B) | Odds ratio for logistic regression(OR ) or Z |
| | (Hazard ratio for coxph regression) or Beta for |
| | linear regression or meta-analysis (B) |
| | If OR < 1 ;(Z < 1); (B < 0) the minor allele |
| | correlates to a reduced risk of a patient |
| | exhibiting the listed phenotype |
| | If OR > 1; (Z > 1); (B > 0), the minor |
| | allele correlates to an increased risk of a |
| | patient exhibiting the listed phenotype |
| P (p_value) | Statistical significance of association between |
| | phenotype and SNP presence |
| BP (base_pair) | Base pair; Genomic location in hg2 |
| | coordinates |
| Chr | Chromosome |
| snp location | Location of SNP on gene |
| Gene (cis_eGene) | Local gene on same chromosome as |
| | the SNP that is differentially regulated in patients |
| | having the related SNP |
| eqtl_beta | Negative value indicates decreased expression |
| | of cis gene in subjects having the related |
| | SNP; positive value indicates increased expression |
| | of cis gene in subjects having the related |
| | SNP |
| eqtl_p | Statistical significance of association between |
| | cisgene and SNP presence |
| priority | Defined as eQTL where the SNP is part of the |
| | cis-eGene that it regulates |

TABLE 7
Description of phenotype abbreviations

| Phenotype/Disease | |
|---|---|
| location | Description |
| L1 | Disease location - ileal |
| L2 | Disease location - colonic |
| L2_colonic | Disease location - colonic |
| L3 | Disease location - ileocolonic |
| B1 | Non-stricturing/non-penetrating |
| B2a | Stricturing |
| B2b | Stricturing and penetrating |
| B2a + B2b | Stricturing with evidence of penetrating |
| B3 | Isolated internal penetrating |
| E Nodosum | Erythema nodosum |
| Paneth-D0 | % normal Paneth cells |
| Paneth-D1 | % abnormal Paneth cells |
| Paneth-D1234 | % abnormal Paneth cells |
| Paneth-D3 | % abnormal Paneth cells |
| Paneth-D4 | % abnormal Paneth cells |
| Paneth-D5 | % abnormal Paneth cells |
| Paneth high (>20%) vs | High-low % abnormal Paneth cells |
| low (<20%) | |
| anti-TNF time to loss | Subjects non-responsive to anti-TNF |
| of response | in months. The loss of response was |
| | characterized by a reappearance of |
| | symptoms consistent with a flare after |
| | initial anti-TNF response, and the |
| | time from induction of therapy to |
| | loss of response was recorded. |
| ANCA | Antineutrophil cytoplasmic antibody |
| Arthralgias | Arthralgias |
| ASCA | Saccharomyces cerevisiae Antibodies |
| IGA ASCA | Saccharomyces cerevisiae Antibodies IGA |
| IGG ASCA | Saccharomyces cerevisiae Antibodies IGG |
| OMPC | Anti-outer-membrane porin C antibody |
| Cbir | Antibody against flagellin |
| i2 | Antibody against Pseudomonas fluorescens |
| PDM | Perianal disease modifier |
| Oral ulcers | Oral ulcers |
| PSC | Primary sclerosing cholangitis |
| Psoriasis | Psoriasis |
| Time to First Surgery | As defined in Soon Man Yoon et al., |
| | “Colonic Phenotypes are Associated with |
| | Poorer Response to Anti-TNF Therapies |
| | in Patients with IBD,” Inflamm. |
| | Bowel Dis., Volume 0, Number 0 (2017) |
| Smoking Status | Current smoker |
| Smokinghx | History of smoking |
| MRUC | Medically Refractory Ulcerative Colitis |
| PyodermaG | pyoderma gangrenosum |
| A_Spondylitis | Spondylitis |
| Arthralgias | Various types of arthralgia |

### Example 6. IL18R1 SNPs Associated with Celiac Disease

A genome-wide association study meta-analysis of 4,533 individuals with celiac disease (cases) and 10,750 controls of European descent were performed on Illumina Infinium HumanHap300 beadchip. A further 231,362 additional non-HLA markers from IlluminaHap550 marker set were tested for association in 3,796 cases and 8,154 controls. Genotype imputation using BEAGLE software and HapMap3 reference samples for samples typed on HumanHap300 chip was performed. After QC SNPs from 113 loci were selected for replication in 4,918 cases and 5,684 controls. All tests for association were conducted using PLINK v1.07 allowing the discovery of 13 novel celiac disease susceptibility loci.

Single nucleotide polymorphisms (SNPs) at the IL18R1 gene or genetic locus was found to be associated with celiac disease in time to celiac disease analyses (10-4>P>5.8×10−6). The hazard ratios (HR) for the SNPs with the smallest P value was 1.45.

### Example 7. IL18R1 SNPs Associated with Primary Billary Cihrosis

Toll-like receptors (TLRs) play a key role in innate immunity. Apart from their function in host defense, dysregulation in TLR signaling can confer risk to autoimmune diseases, septic shock or cancer. SNPs conferring risk to primary biliary cirrhosis (PBC), inflammatory bowel disease (IBD) and celiac disease were found to be immune response eQTLs for IL18R1. Thus, IL18R1 represents a plausible candidate for studying the pathophysiology of these disorders in the context of TLR4 activation.

### Example 8. IL18R1 SNPs Associated with Asthma

IL18R1 has been implicated in the pathophysiology of asthma and maps to an asthma susceptibility locus on chromosome 2q12. The possibility of association between polymorphisms in IL18R1 and asthma was examined by genotyping seven SNPs in 294, 342 and 100 families from Denmark, United Kingdom and Norway and conducting family-based association analyses for asthma, atopic asthma and bronchial hyper-reactivity (BHR) phenotypes. Three SNPs in IL18R1 were associated with asthma (0.01131< or =P< or =0.01377), five with atopic asthma (0.00066< or =P < or =0.00405) and two with BHR (0.01450< or =P< or =0.03203) in the Danish population; two SNPs were associated with atopic asthma (0.00397< or =P< or =0.01481) and four with BHR (0.00435< or =P< or =0.03544) in the UK population; four SNPs showed associations with asthma (0.00015< or =P< or =0.03062), two with atopic asthma (0.01269< or =P< or =0.04042) and three with BHR (0.00259< or =P< or =0.01401) in the Norwegian population; five SNPs showed associations with asthma (0.00005< or =P< or =0.03744), five with atopic asthma (0.00001< or =P< or =0.04491) and three with BHR (0.03568< or =P< or =0.04778) in the combined population. Three intronic SNPs (rs1420099, rs1362348 and rs1974675) showed replicated association for at least one asthma-related phenotype. These results demonstrate significant association between polymorphisms in IL18R1 and asthma.

### Example 9: Phase 1A Clinical Trial

A phase 1A clinical trial is performed to evaluate the safety, tolerability, pharmacokinetics, and pharmacodynamics of a compound disclosed herein, e.g., an modulator of IL18R1, in subjects with moderate to severely active Crohn's disease. Eligible subjects are men and women 18 years and older. Optionally, two groups of subjects are selected: (i) subjects having an IL18R1 risk genotype comprising one or more of rs1921622, rs2287037, rs1974675, rs2041739, rs76362690, rs2287037, or rs80256362, or a SNP or indel in linkage disequilibrium (LD) therewith; and (ii) subjects lacking the genotype.

Inclusion Criteria: Eligible subjects are men and women 18 years and older. Two groups of subjects are selected: (i) subjects having a genotype comprising one or more of rs1921622, rs2287037, rs1974675, rs2041739, rs76362690, rs2287037, or rs80256362, or a SNP or indel in linkage disequilibrium (LD) therewith; and (ii) subjects lacking the genotype. Subjects are patients with Crohn's disease of at least 3 months' duration, confirmed at any time in the past by radiography, histology, and/or endoscopy. Female patient of childbearing potential must have a negative highly sensitive serum (beta-human chorionic gonadotropin [b-hCG]) pregnancy test result at screening and a negative urine pregnancy test result at Week 0. Subjects must adhere to the following requirements for concomitant medication for the treatment of Crohn's disease, which are permitted provided that doses meeting these requirements are stable, or have been discontinued, for at least 3 weeks before baseline (Week 0), unless otherwise specified: a) Oral 5-aminosalicylic acid (5-ASA) compounds, b) Oral corticosteroids at a prednisone-equivalent dose at or below 40 milligram per day (mg/day), or 9 mg/day of budesonide, or 5 mg/day beclomethasone dipropionate, c) Antibiotics being used as a primary treatment of Crohn's disease, d) Conventional immunomodulators (that is, azathioprine (AZA), 6-mercaptopurine (6-MP), or Methotrexate (MTX)): participants must have been taking them for at least 12 weeks and at a stable dose for at least 4 weeks before baseline. Subjects who has or had extensive colitis for greater than or equal to (>=) 8 years, or disease limited to the left side of the colon for >=12 years, must either have had a colonoscopy to assess for the presence of dysplasia within 1 year before the first administration of study agent or a colonoscopy to assess for the presence of malignancy at the screening visit, with no evidence of malignancy. Subjects must have active Crohn's disease, defined as a baseline Crohn's Disease Activity Index (CDAI) score of >=220 but <=450.

Experimental (Part I): Placebo. Subjects will receive placebo at Weeks 0, 2, 4, and 6. From Week 8 Placebo-treated subjects who are in clinical response at Week 8 (>=100-point reduction from baseline in Crohn's Disease Activity Index (CDAI) or CDAI <150) will continue to receive placebo every 2 weeks from Week 8 through Week 12. Placebo-treated subjects who are not in clinical response at Week 8 will receive test compound (a compound described herein) 400 mg at Week 8 and then test compound every two weeks from Week 10 through Week 12.

Experimental (Part I): Placebo. Test Compound. Subjects will receive test compound 400 milligram (mg) at Week 0 then 200 mg every two weeks through Week 22.

Experimental (Part II): Placebo. Placebo at Weeks 0, 2, 4, and 8. From Week 12, placebo-treated subjects who are in clinical response at Week 8 (>=100-point reduction from baseline in CDAI or CDAI <150) will continue to receive placebo at Weeks 8, 10, and 12. Placebo-treated subjects who are not in clinical response at Week 8 will receive test compound 150 mg at Week 8 and then test compound 75 mg at Weeks 10, and 12.

Experimental (Part II): Test Compound High Dose. Test compound 400 mg at Week 0 and 200 mg at Weeks 2, 4, 8, and 12.

Experimental (Part II) Test Compound Middle Dose. Test compound 150 mg at Week 0 and 75 mg at Weeks 2, 4, 8, and 12.

Experimental (Part II) Test compound Low Dose. Test compound 50 mg at Week 0 and 25 mg at Weeks 2, 4, 8, and 12.

Primary Outcome Measures: Part I: Change From Baseline in the Crohn's Disease Activity Index (CDAI) Score at Week 8 [Time Frame: Baseline through Week 8]—CDAI will be assessed by collecting information on 8 different Crohn's disease-related variables: extra-intestinal manifestations, abdominal mass, weight, hematocrit, total number of liquid stools, abdominal pain/cramping, use of antidiarrheal drug(s) and/or opiates, and general well-being. The last 4 variables are scored over 7 days by the participant on a diary card. Part II: Change From Baseline in the Crohn's Disease Activity Index (CDAI) Score at Week 8 [Time Frame: Baseline through Week 8]—CDAI are assessed by collecting information on 8 different Crohn's disease-related variables: extra-intestinal manifestations, abdominal mass, weight, hematocrit, total number of liquid stools, abdominal pain/cramping, use of antidiarrheal drug(s) and/or opiates, and general well-being. The last 4 variables are scored over 7 days by the participant on a diary card.

Secondary Outcome Measures: Part II: Clinical Remission at Week 8 as Measured by Crohn's Disease Activity Index (CDAI <150) [Time Frame: Week 8]. Part II: Clinical Response at Week 8 as Measured by CDAI (>=100-point reduction from baseline in CDAI or CDAI <150) [Time Frame: Week 8]. Part II: Change in Patient-Reported Outcome (PRO)-2 from baseline at Week 8 [Time Frame: Baseline through Week 8]—The PRO-2 score is the sum of the abdominal pain and stool frequency subscores of the CDAI score. Part II: Clinical remission at Week 8 as measured by PRO-2 (PRO-2<75) [Time Frame: Week 8]. Part II: Clinical response at Week 8 as measured by PRO-2 (>=50-point reduction from baseline in PRO-2 or PRO-2<75) [Time Frame: Week 8]. Part II: Change in Simple Endoscopic Score for Crohn's Disease (SES-CD) from baseline at Week 12 [Time Frame: Baseline through Week 12]—The SES-CD score is based on the evaluation of 4 endoscopic components (presence/size of ulcers, proportion of mucosal surface covered by ulcers, proportion of mucosal surface affected by any other lesions, and presence/type of narrowing/strictures) across 5 ileocolonic segments. Each endoscopic component is scored from 0 to 3 for each segment, and a total score is derived from the sum of all the component scores (range, 0 to 56).

### Example 10: Phase 1B Clinical Trial

A phase 1B clinical trial is performed to evaluate the efficacy of a compound described herein comprising an modulator of IL18R1, in participants with moderately to severely active Crohn's disease that have a genotype comprising one or more of rs3915617, rs1064448 rs1872691, rs2302712, rs3760012.

Experimental. 10 patients positive for rs1921622, rs2287037, rs1974675, rs2041739, rs76362690, rs2287037, or rs80256362, or a SNP or indel in linkage disequilibrium (LD) therewith, are administered the modulator of IL18R1. 5-10 patients negative for rs1921622, rs2287037, rs1974675, rs2041739, rs76362690, rs2287037, or rs80256362, or a SNP or indel in linkage disequilibrium (LD) therewith, are administered the modulator of IL18R1. Patients are monitored in real-time. Central ready of endoscopy and biopsy is employed, with readers blinded to point of time of treatment and endpoints.

Inclusion Criteria: Two groups of subjects are selected: (i) subjects having the rs1921622, rs2287037, rs1974675, rs2041739, rs76362690, rs2287037, or rs80256362, or a SNP or indel in linkage disequilibrium (LD) therewith genotype, and (ii) subjects lacking the rs1921622, rs2287037, rs1974675, rs2041739, rs76362690, rs2287037, or rs80256362, or a SNP or indel in linkage disequilibrium (LD) therewith genotype.

Primary Outcome Measures: Simple Endoscopic Score for Crohn's Disease (SESCD), Crohn's Disease Activity Index (CDAI), and Patient Reported Outcome (PRO). If the rs1921622, rs2287037, rs1974675, rs2041739, rs76362690, rs2287037, or rs80256362, or a SNP or indel in linkage disequilibrium (LD) therewith genotype positive group shows at least 20% reduction from baseline, a Phase 2a clinical trial is performed.

Inclusion Criteria: PRO entry criteria: Abdominal pain score of 2 or more and/or stool frequency score of 4 or more. Primary outcome would be pain core of 0 or 1 and stool frequency score of 3 or less with no worsening from baseline. Endoscopy entry criteria: SESCD ileum only entry at score of 4 and 6 if colon is involved. Primary endoscopic outcome is 40-50% delta of mean SESCD.

### Example 11: Phase 2A Clinical Trial

A phase 2A clinical trial is performed to evaluate efficacy of a compound disclosed herein, e.g., an modulator of IL18R1, in subjects having a genotype comprising rs1921622, rs2287037, rs1974675, rs2041739, rs76362690, rs2287037, or rs80256362, or a SNP or indel in linkage disequilibrium (LD) therewith, with moderately to severely active Crohn's disease.

Experimental. 40 patients (drug and placebo groups) positive for rs1921622, rs2287037, rs1974675, rs2041739, rs76362690, rs2287037, or rs80256362, or a SNP or indel in linkage disequilibrium (LD) therewith are administered the modulator of IL18R1 or placebo for 12 weeks. An interim analysis is performed after 20 patients from each group are treated at the highest dose to look for 40-50% delta between placebo and treated group in primary outcome (at least 20% reduction from baseline in SESCD, CDAI, and PRO).

Primary Outcome Measures: Simple Endoscopic Score for Crohn's Disease (SESCD), Crohn's Disease Activity Index (CDAI), and Patient Reported Outcome (PRO).

Inclusion Criteria: PRO entry criteria: Abdominal pain score of 2 or more and/or stool frequency score of 4 or more. Primary outcome would be pain core of 0 or 1 and stool frequency score of 3 or less with no worsening from baseline. Endoscopy entry criteria: SESCD ileum only entry at score of 4 and 6 if colon is involved. Primary endoscopic outcome is 40-50% delta of mean SESCD.

### Example 12. Treating Inflammatory Disease

An inflammatory disease is treated in a subject, by first, determining the IL18R1 risk genotype of the subject. Optionally, the subject is, or is susceptible to be, non-responsive to certain therapies such as anti-TNF, steroids, or immunomodulators, such as those disclosed herein. A sample of whole blood is obtained from the subject. An assay is performed on the sample obtained from the subject to detect a presence or absence of rs1921622, rs2287037, rs1974675, rs2041739, rs76362690, rs2287037, or rs80256362, or a SNP or indel in linkage disequilibrium (LD) therewith, by Illumina ImmunoArray or polymerase chain reaction (PCR) under standard hybridization conditions. In addition, or alternatively, a sample of intestinal tissue is obtained from the subject. In addition, or alternatively, a sample of intestinal tissue is obtained from the subject. An assay is performed on the sample obtained from the subject to detect a presence of IL18R1, by single molecule detection (e.g., Simoa) accordingly to manufacture instructions.

The subject is determined to have, or be at risk for developing, moderate to severe Crohn's disease if the IL18R1 risk genotype comprising rs1921622, rs2287037, rs1974675, rs2041739, rs76362690, rs2287037, or rs80256362, or a SNP or indel in linkage disequilibrium (LD) therewith is detected in the sample obtained from the subject. A therapeutically effective amount of a modulator of IL18R1 is administered to the subject, provided the subject is determined to have the IL18R1 risk genotype.

### Example 13: In Vitro and In Vivo Studies

Reagent validation in vitro: Patient samples are analyzed to determine IL18R1 expression and to perform a preliminary functional analysis comprising testing IL-18 induction of IL-1, IL-6 and MAPK p38 phosphorylation, controlling for IL-1beta activity.

In vivo validation: IL18R1-deficient mice from Jackson laboratories are utilized to evaluate disease phenotype and gene expression.

### Example 14. IL18R1 Sequences

mRNA and protein sequences for IL18R1 are listed in Tables 8-9.

TABLE 8
IL18R1 mRNA Sequences

| SEQ | NCBI | | |
|---|---|---|---|
| ID | Ref. | | |
| NO. | No. | Variant | Sequence |
| 10 | NM_003855.3 | 1 | TCAGGAGGCGGAGATCGCTGCTTCTCACCTACTTTCTGAACTTG |
| | | | GCCTCCGCAGTCGCGACCTGGCGTGAAGGAGGAGCTGCCGCCC |
| | | | CCGCCCCAGCCTCGGGGACGCCTCTCTGAAGAGAAGCCATTTG |
| | | | AAGCAGAATCCAAACCATGAATTGTAGAGAATTACCCTTGACC |
| | | | CTTTGGGTGCTTATATCTGTAAGCACTGCAGAATCTTGTACTTC |
| | | | ACGTCCCCACATTACTGTGGTTGAAGGGGAACCTTTCTATCTG |
| | | | AAACATTGCTCGTGTTCACTTGCACATGAGATTGAAACAACCA |
| | | | CCAAAAGCTGGTACAAAAGCAGTGGATCACAGGAACATGTGG |
| | | | AGCTGAACCCAAGGAGTTCCTCGAGAATTGCTTTGCATGATTG |
| | | | TGTTTTGGAGTTTTGGCCAGTTGAGTTGAATGACACAGGATCTT |
| | | | ACTTTTTCCAAATGAAAAATTATACTCAGAAATGGAAATTAAA |
| | | | TGTCATCAGAAGAAATAAACACAGCTGTTTCACTGAAAGACAA |
| | | | GTAACTAGTAAAATTGTGGAAGTTAAAAAATTTTTTCAGATAA |
| | | | CCTGTGAAAACAGTTACTATCAAACACTGGTCAACAGCACATC |
| | | | ATTGTATAAGAACTGTAAAAAGCTACTACTGGAGAACAATAAA |
| | | | AACCCAACGATAAAGAAGAACGCCGAGTTTGAAGATCAGGGG |
| | | | TATTACTCCTGCGTGCATTTCCTTCATCATAATGGAAAACTATT |
| | | | TAATATCACCAAAACCTTCAATATAACAATAGTGGAAGATCGC |
| | | | AGTAATATAGTTCCGGTTCTTCTTGGACCAAAGCTTAACCATGT |
| | | | TGCAGTGGAATTAGGAAAAAACGTAAGGCTCAACTGCTCTGCT |
| | | | TTGCTGAATGAAGAGGATGTAATTTATTGGATGTTCGGGGAAG |
| | | | AAAATGGATCGGATCCTAATATACATGAAGAGAAAGAAATGA |
| | | | GAATTATGACTCCAGAAGGCAAATGGCATGCTTCAAAAGTATT |
| | | | GAGAATTGAAAATATTGGTGAAAGCAATCTAAATGTTTTATAT |
| | | | AATTGCACTGTGGCCAGCACGGGAGGCACAGACACCAAAAGC |
| | | | TTCATCTTGGTGAGAAAAGCAGACATGGCTGATATCCCAGGCC |
| | | | ACGTCTTCACAAGAGGAATGATCATAGCTGTTTTGATCTTGGT |
| | | | GGCAGTAGTGTGCCTAGTGACTGTGTGTGTCATTTATAGAGTT |
| | | | GACTTGGTTCTATTTTATAGACATTTAACGAGAAGAGATGAAA |
| | | | CATTAACAGATGGAAAAACATATGATGCTTTTGTGTCTTACCT |
| | | | AAAAGAATGCCGACCTGAAAATGGAGAGGAGCACACCTTTGC |
| | | | TGTGGAGATTTTGCCCAGGGTGTTGGAGAAACATTTTGGGTAT |
| | | | AAGTTATGCATATTTGAAAGGGATGTAGTGCCTGGAGGAGCTG |
| | | | TTGTTGATGAAATCCACTCACTGATAGAGAAAAGCCGAAGACT |
| | | | AATCATTGTCCTAAGTAAAAGTTATATGTCTAATGAGGTCAGG |
| | | | TATGAACTTGAAAGTGGACTCCATGAAGCATTGGTGGAAAGAA |
| | | | AAATTAAAATAATCTTAATTGAATTTACACCTGTTACTGACTTC |
| | | | ACATTCTTGCCCCAATCACTAAAGCTTTTGAAATCTCACAGAGT |
| | | | TCTGAAGTGGAAGGCCGATAAATCTCTTTCTTATAACTCAAGG |
| | | | TTCTGGAAGAACCTTCTTTACTTAATGCCTGCAAAAACAGTCA |
| | | | AGCCAGGTAGAGACGAACCGGAAGTCTTGCCTGTTCTTTCCGA |
| | | | GTCTTAATCTTCAGAAACAGTGAACGCCAAAAAGAACTCAAGA |
| | | | TATTCTGGGGACTGAGCATATGAACCTGTTCATAACAAAGGCT |
| | | | GTGACTCGAAATAATTAACTTTGTCAAAATCCTGCTCACAATTT |
| | | | GAAGATGAAACTTGTCATTAGGTTGGCGGGAATGAGACTAAA |
| | | | GATTGCGCTGTGGGCTGTGGTCACGTGCTCCCAGAAGACCTGG |
| | | | AATTCAAAAGAAATGGAGCTATTCTTTTTCTCCCTCTTTCATAA |
| | | | CTGGATGCAGCTGCTCATACTCAATCCCATATTCAGCAAGTGT |
| | | | GAAGCTGGACGTGATGCAAAATAACCGATGCCCTACAAAAAG |
| | | | GGCGCATCTTTAAGAGTTTTAATGCCAGTGCTTAATTCGAATG |
| | | | AGGGGATTTTAAGTGTCTGAAGAGGCATTTTCTAGGGACCAGT |
| | | | GGGTGACTGAGTAACTGAAATGCTGCTTTCACTCCCTAACACC |
| | | | ATGGATCTGGTTGTGCATAGGATGTGGGAGGAGGGGCTGGCA |
| | | | GGGCCGCCTTCAGAGGCTGCAGGGCCTCAGCCTCAGGATGCAT |
| | | | TTAATGTATCCTGGCCACAGTTGCAGCCAACGGTTCTTGAAAG |
| | | | CTCGGTAAGGCCCTGCAACGCAGAGCCTGCTTATGTGGATCTA |
| | | | TTTATGGGAACTTCTTAAAAGGACCCCAGAATAGCTCTTTATCT |
| | | | TTCACAAGAGACACAAATTCTAATTGAGTTAATTATCTGGGCC |
| | | | TTTCACTTTGGATGCTCTGAAACATTTGTTGATTTTGTGTGAAT |
| | | | GTTTATATCAAAATGTTTGCCAGGTTGTATTAGCCATTGAATAG |
| | | | CAAAAAACTGATAGTTACTTGCTTGTTTTTTAAAAATTACATAT |
| | | | TAAAAATGCCCTTGGCATAAGGCAGCATGGTGTGGCAGTTAAG |
| | | | AGATGGGCTGTGCAGCCCATCCTGAGCTCCAGTCCTGAGTTTG |
| | | | CTACTTACTTCTGTGGCCTCTGGAACCTTATCCAACCTCTTGGT |
| | | | GCTTCAGTTTCCTCATCTGTGAAATTAGAATTTATAATAATTGC |
| | | | ACCTACCTCCCAGGGGTAACTAAATGAATAAATATAATAAAGT |
| | | | ACTTACAGTGGTTCCTGACACAGACTCAGCACTCCGTCAGTGT |
| | | | TGCCATGACTATTTTTATTATCATTATTAATGATTACTTAGATC |
| | | | AATTATTTAGCAGTGGACTAATGGAAGCTACAGAGCAGGGAA |
| | | | GGGAAGCAGATCTAGGGAGGAAGGCAGTTTTGATTTGAGGAG |
| | | | GTTTGCACATGTAGAGAAGCATACTGGAGAAGCATATCCAGAG |
| | | | GGCGAAAGATATCTCTCCATTGTGCATCTGCCTCTTTTGACGTT |
| | | | GGAAGACACATGTCTTACTCCCCAAAGGGAGCCCAGCACTGGG |
| | | | AGCCTTCTTGATGATCTCAAAAATAATAGCTATTCAAGAAAAT |
| | | | CACCAAGTGACTGTGAAACCGTCAGTTCGGAAGGCTGGTTAGA |
| | | | ACATGTGGGAGCAACATGAATGTTCTACAAAAGTTTAAAGCAG |
| | | | AGATTGTTTCAAATGGGTGTAGTAGATATTACTGAAAACCAAA |
| | | | AAAGAGTGAGATTGTCAGTGTAAGAATGTGATTTAATGTTTGT |
| | | | AGTGCTTACAATTTTGTGTACCAACTGGATGACTAAAAAGAGT |
| | | | AAAATAATTTAATTAATAGCTCATATTTTATGTGTGAAAACAT |
| | | | GTTAGTGAACATATATAATCAAAATAGATTTCATTGCTATTGC |
| | | | ATAGTCTCTAATACATAGAATGATTTTGCTTTTCTCTTTTATTAT |
| | | | ACTTGCTTTAAAATACTTGAAATATATTTTGCATTAAATGCATT |
| | | | TCAAGTTAAATGTCTTAAATGTATACATTAGATGTGTGTTTTAA |
| | | | AATGCATAAAACACGTTGAAATACATTAATGAACCATT |
| 11 | NM_001282399.1 | 2 | TCAGGAGGCGGAGATCGCTGCTTCTCACCTACTTTCTGAACTTG |
| | | | GCCTCCGCAGTCGCGACCTGGCGTGAAGGAGGAGCTGCCGCCC |
| | | | CCGCCCCAGCCTCGGGGACGCCTCTCTGAAGAGAAGCCATTTG |
| | | | AAGCAGAATCCAAACCATGAATTGTAGAGAATTACCCTTGACC |
| | | | CTTTGGGTGCTTATATCTGTAAGCACTGCAGAAATTATACTCAG |
| | | | AAATGGAAATTAAATGTCATCAGAAGAAATAAACACAGCTGTT |
| | | | TCACTGAAAGACAAGTAACTAGTAAAATTGTGGAAGTTAAAA |
| | | | AATTTTTTCAGATAACCTGTGAAAACAGTTACTATCAAACACT |
| | | | GGTCAACAGCACATCATTGTATAAGATAGGACCACCTATTTGC |
| | | | AGGAAAACAAGCTCAGGGCTCCACTGATTCTACATTATGAACT |
| | | | GTAAAAAGCTACTACTGGAGAACAATAAAAACCCAACGATAA |
| | | | AGAAGAACGCCGAGTTTGAAGATCAGGGGTATTACTCCTGCGT |
| | | | GCATTTCCTTCATCATAATGGAAAACTATTTAATATCACCAAA |
| | | | ACCTTCAATATAACAATAGTGGAAGATCGCAGTAATATAGTTC |
| | | | CGGTTCTTCTTGGACCAAAGCTTAACCATGTTGCAGTGGAATT |
| | | | AGGAAAAAACGTAAGGCTCAACTGCTCTGCTTTGCTGAATGAA |
| | | | GAGGATGTAATTTATTGGATGTTCGGGGAAGAAAATGGATCGG |
| | | | ATCCTAATATACATGAAGAGAAAGAAATGAGAATTATGACTCC |
| | | | AGAAGGCAAATGGCATGCTTCAAAAGTATTGAGAATTGAAAA |
| | | | TATTGGTGAAAGCAATCTAAATGTTTTATATAATTGCACTGTGG |
| | | | CCAGCACGGGAGGCACAGACACCAAAAGCTTCATCTTGGTGA |
| | | | GAAAAGACATGGCTGATATCCCAGGCCACGTCTTCACAAGAGG |
| | | | AATGATCATAGCTGTTTTGATCTTGGTGGCAGTAGTGTGCCTAG |
| | | | TGACTGTGTGTGTCATTTATAGAGTTGACTTGGTTCTATTTTAT |
| | | | AGACATTTAACGAGAAGAGATGAAACATTAACAGATGGAAAA |
| | | | ACATATGATGCTTTTGTGTCTTACCTAAAAGAATGCCGACCTG |
| | | | AAAATGGAGAGGAGCACACCTTTGCTGTGGAGATTTTGCCCAG |
| | | | GGTGTTGGAGAAACATTTTGGGTATAAGTTATGCATATTTGAA |
| | | | AGGGATGTAGTGCCTGGAGGAGCTGTTGTTGATGAAATCCACT |
| | | | CACTGATAGAGAAAAGCCGAAGACTAATCATTGTCCTAAGTAA |
| | | | AAGTTATATGTCTAATGAGGTCAGGTATGAACTTGAAAGTGGA |
| | | | CTCCATGAAGCATTGGTGGAAAGAAAAATTAAAATAATCTTAA |
| | | | TTGAATTTACACCTGTTACTGACTTCACATTCTTGCCCCAATCA |
| | | | CTAAAGCTTTTGAAATCTCACAGAGTTCTGAAGTGGAAGGCCG |
| | | | ATAAATCTCTTTCTTATAACTCAAGGTTCTGGAAGAACCTTCTT |
| | | | TACTTAATGCCTGCAAAAACAGTCAAGCCAGGTAGAGACGAA |
| | | | CCGGAAGTCTTGCCTGTTCTTTCCGAGTCTTAATCTTCAGAAAC |
| | | | AGTGAACGCCAAAAAGAACTCAAGATATTCTGGGGACTGAGC |
| | | | ATATGAACCTGTTCATAACAAAGGCTGTGACTCGAAATAATTA |
| | | | ACTTTGTCAAAATCCTGCTCACAATTTGAAGATGAAACTTGTC |
| | | | ATTAGGTTGGCGGGAATGAGACTAAAGATTGCGCTGTGGGCTG |
| | | | TGGTCACGTGCTCCCAGAAGACCTGGAATTCAAAAGAAATGGA |
| | | | GCTATTCTTTTTCTCCCTCTTTCATAACTGGATGCAGCTGCTCAT |
| | | | ACTCAATCCCATATTCAGCAAGTGTGAAGCTGGACGTGATGCA |
| | | | AAATAACCGATGCCCTACAAAAAGGGCGCATCTTTAAGAGTTT |
| | | | TAATGCCAGTGCTTAATTCGAATGAGGGGATTTTAAGTGTCTG |
| | | | AAGAGGCATTTTCTAGGGACCAGTGGGTGACTGAGTAACTGAA |
| | | | ATGCTGCTTTCACTCCCTAACACCATGGATCTGGTTGTGCATAG |
| | | | GATGTGGGAGGAGGGGCTGGCAGGGCCGCCTTCAGAGGCTGC |
| | | | AGGGCCTCAGCCTCAGGATGCATTTAATGTATCCTGGCCACAG |
| | | | TTGCAGCCAACGGTTCTTGAAAGCTCGGTAAGGCCCTGCAACG |
| | | | CAGAGCCTGCTTATGTGGATCTATTTATGGGAACTTCTTAAAA |
| | | | GGACCCCAGAATAGCTCTTTATCTTTCACAAGAGACACAAATT |
| | | | CTAATTGAGTTAATTATCTGGGCCTTTCACTTTGGATGCTCTGA |
| | | | AACATTTGTTGATTTTGTGTGAATGTTTATATCAAAATGTTTGC |
| | | | CAGGTTGTATTAGCCATTGAATAGCAAAAAACTGATAGTTACT |
| | | | TGCTTGTTTTTTAAAAATTACATATTAAAAATGCCCTTGGCATA |
| | | | AGGCAGCATGGTGTGGCAGTTAAGAGATGGGCTGTGCAGCCC |
| | | | ATCCTGAGCTCCAGTCCTGAGTTTGCTACTTACTTCTGTGGCCT |
| | | | CTGGAACCTTATCCAACCTCTTGGTGCTTCAGTTTCCTCATCTG |
| | | | TGAAATTAGAATTTATAATAATTGCACCTACCTCCCAGGGGTA |
| | | | ACTAAATGAATAAATATAATAAAGTACTTACAGTGGTTCCTGA |
| | | | CACAGACTCAGCACTCCGTCAGTGTTGCCATGACTATTTTTATT |
| | | | ATCATTATTAATGATTACTTAGATCAATTATTTAGCAGTGGACT |
| | | | AATGGAAGCTACAGAGCAGGGAAGGGAAGCAGATCTAGGGAG |
| | | | GAAGGCAGTTTTGATTTGAGGAGGTTTGCACATGTAGAGAAGC |
| | | | ATACTGGAGAAGCATATCCAGAGGGCGAAAGATATCTCTCCAT |
| | | | TGTGCATCTGCCTCTTTTGACGTTGGAAGACACATGTCTTACTC |
| | | | CCCAAAGGGAGCCCAGCACTGGGAGCCTTCTTGATGATCTCAA |
| | | | AAATAATAGCTATTCAAGAAAATCACCAAGTGACTGTGAAACC |
| | | | GTCAGTTCGGAAGGCTGGTTAGAACATGTGGGAGCAACATGA |
| | | | ATGTTCTACAAAAGTTTAAAGCAGAGATTGTTTCAAATGGGTG |
| | | | TAGTAGATATTACTGAAAACCAAAAAAGAGTGAGATTGTCAGT |
| | | | GTAAGAATGTGATTTAATGTTTGTAGTGCTTACAATTTTGTGTA |
| | | | CCAACTGGATGACTAAAAAGAGTAAAATAATTTAATTAATAGC |
| | | | TCATATTTTATGTGTGAAAACATGTTAGTGAACATATATAATCA |
| | | | AAATAGATTTCATTGCTATTGCATAGTCTCTAATACATAGAATG |
| | | | ATTTTGCTTTTCTCTTTTATTATACTTGCTTTAAAATACTTGAAA |
| | | | TATATTTTGCATTAAATGCATTTCAAGTTAAATGTCTTAAATGT |
| | | | ATACATTAGATGTGTGTTTTAAAATGCATAAAACACGTTGAAA |
| | | | TACATTAATGAACCATT |

TABLE 9
IL18R1 Protein Sequences

| SEQ | NCBI | | |
|---|---|---|---|
| ID | Ref. | | |
| NO. | No. | Isoform | Sequence |
| 8 | NP_03846.1 | 1 | MNCRELPLTLWVLISVSTAESCTSRPHITVVEGEPFYLKHCSCSLA |
| | | | HEIETTTKSWYKSSGSQEHVELNPRSSSRIALHDCVLEFWPVELND |
| | | | TGSYFFQMKNYTQKWKLNVIRRNKHSCFTERQVTSKIVEVKKFF |
| | | | QITCENSYYQTLVNSTSLYKNCKKLLLENNKNPTIKKNAEFEDQG |
| | | | YYSCVHFLHHNGKLFNITKTFNITIVEDRSNIVPVLLGPKLNHVAV |
| | | | ELGKNVRLNCSALLNEEDVIYWMFGEENGSDPNIHEEKEMRIMTP |
| | | | EGKWHASKVLRIENIGESNLNVLYNCTVASTGGTDTKSFILVRKA |
| | | | DMADIPGHVFTRGMIIAVLILVAVVCLVTVCVIYRVDLVLFYRHL |
| | | | TRRDETLTDGKTYDAFVSYLKECRPENGEEHTFAVEILPRVLEKH |
| | | | FGYKLCIFERDVVPGGAVVDEIHSLIEKSRRLIIVLSKSYMSNEVRY |
| | | | ELESGLHEALVERKIKIILIEFTPVTDFTFLPQSLKLLKSHRVLKWK |
| | | | ADKSLSYNSRFWKNLLYLMPAKTVKPGRDEPEVLPVLSES |
| 9 | NP_01269328.1 | 2 | MNCKKLLLENNKNPTIKKNAEFEDQGYYSCVHFLHHNGKLFNIT |
| | | | KTFNITIVEDRSNIVPVLLGPKLNHVAVELGKNVRLNCSALLNEED |
| | | | VIYWMFGEENGSDPNIHEEKEMRIMTPEGKWHASKVLRIENIGES |
| | | | NLNVLYNCTVASTGGTDTKSFILVRKDMADIPGHVFTRGMIIAVLI |
| | | | LVAVVCLVTVCVIYRVDLVLFYRHLTRRDETLTDGKTYDAFVSY |
| | | | LKECRPENGEEHTFAVEILPRVLEKHFGYKLCIFERDVVPGGAVV |
| | | | DEIHSLIEKSRRLIIVLSKSYMSNEVRYELESGLHEALVERKIKIILIE |
| | | | FTPVTDFTFLPQSLKLLKSHRVLKWKADKSLSYNSRFWKNLLYL |
| | | | MPAKTVKPGRDEPEVLPVLSES |

TABLE 10
Other Sequences

| SEQ ID NO. | Type of Sequence | Sequence |
|---|---|---|
| 1 | Nucleic Acid | ACTTCTTAAT TCTGTCCATA AGATTTGAAA |
| | | GAGGACTTAA AAATTGATGA N TTTTGTTCTG |
| | | GTAGCCATAG GCACTAGCTG AAATACCTTA |
| | | AAAGTACTCA |
| 2 | Nucleic Acid | AAAACAGATT CAGCCAAAGC TTTCAAACAA |
| | | AAGTGTGCCT ATCTTATGAA N GTTTAAAAAT |
| | | CTTCTGGCAC ACAGATTTTT AAAAAAACAA |
| | | CCTAGAAGAT |
| 3 | Nucleic Acid | TCTGTGTGTA CATTTCCCTC TACCTTCATT |
| | | TCTTCATCTC TATCATTGAA AA N |
| | | TATCCCTTTT GTATCCCCTT CCTTCTTAGT |
| | | TGATTTCGTT CTTAAAATTT |
| 4 | Nucleic Acid | TAATAGACCC TGAAGTTTCC CACATCCTAC |
| | | TCCTGAGTTC CTGTGAATAC N ATATGTTACA |
| | | TGGCAAAAGG AACCTCTAGG TGTGATTAAA |
| | | TTAAGGATCT |
| 5 | Nucleic Acid | CAAAACTGTA ACAAAATTAA GAAAAAGCTG |
| | | GTTCAATGAG CTTAGATTCT N TGAGATTAAT |
| | | CTGAAAAGGG AGAGTAGTTA TGAGAAGTCT |
| | | TAAAAAAGTG |
| 6 | Nucleic Acid | AAAACAGATT CAGCCAAAGC TTTCAAACAA |
| | | AAGTGTGCCT ATCTTATGAA N GTTTAAAAAT |
| | | CTTCTGGCAC ACAGATTTTT AAAAAAACAA |
| | | CCTAGAAGAT |
| 7 | Nucleic Acid | CTCTCAAATG CCTCCTGAAT CACTGGGATT |
| | | CCTTTGAGGA AAAAAGAAAA N GCCTTCTTTC |
| | | CCCTTTTGCC TCCTCTGTCC TCTCTTCACA |
| | | GATGGGTAAT |

While preferred embodiments of the present examples have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### Additional Embodiments

1. A method of treating or preventing a disease or condition in a subject, the method comprising administering a modulator of Interleukin 18 Receptor 1(IL18R1) activity or expression to the subject, provided a genotype is detected in a sample obtained the subject.
2. A method of reducing or ablating activity or expression of Interleukin 18 Receptor 1(IL18R1) in a subject, the method comprising administering a modulator of IL18R1 to the subject, provided a genotype is detected in a sample obtained from the subject.
3. A method of treating or preventing a disease or condition in a subject, the method comprising:
   a) obtaining a sample from a subject;
   b) detecting a presence or an absence of a genotype in the sample obtained from the subject; and
   c) administering to the subject a modulator of Interleukin 18 Receptor 1(IL18R1) activity or expression to the subject, provided the presence of the genotype is detected in the sample obtained from the subject.
4. A method of reducing, ablating, increasing, or activating, an activity or expression of Interleukin 18 Receptor 1 (IL18R1) in a subject, the method comprising:
   a) obtaining a sample from a subject;
   b) detecting a presence or an absence of a genotype in the sample obtained from the subject; and
   c) administering to the subject a modulator of IL18R1 activity or expression to the subject, provided the presence of the genotype is detected in the sample obtained from the subject.
5. The method of any one or claims 1-4, wherein the genotype is detected with an assay comprising polymerase chain reaction (PCR), quantitative reverse-transcription PCR (qPCR), automated sequencing, genotype array, or a combination thereof.
6. The method of any one of claims 1-5, wherein the modulator of IL18R1 activity or expression comprises an agonist or a partial agonist of IL18R1.
7. The method of any one of claims 1-5, wherein the modulator of IL18R1 activity or expression comprises an antagonist or partial antagonist of IL18R1.
8. The method of claim 6 or 7, wherein the agonist or partial agonist comprises an antibody or antigen-binding fragment, peptide, small molecule.
9. The method of any one of claims 6-7, wherein the modulator of IL18R1 activity or expression comprises an inverse agonist.
10. The method of any one of claims 6-7, wherein the modulator of IL18R1 activity or expression comprises a positive allosteric modulator (PAM).
11. The method of any one of claims 6-7, wherein the modulator of IL18R1 activity or expression comprises a negative allosteric modulator (NAM).
12. The method of any one of claims 6-7, wherein the modulator of IL18R1 activity or expression comprises a small molecule that binds to IL18R1 or IL18, or both.
13. The method of any one of claims 6-7, wherein the modulator of IL18R1 activity or expression comprises an antibody or antigen binding fragment that binds to IL18R1 or IL18, or both.
14. The method of any one of claims 6-7, wherein the modulator of IL18R1 activity or expression comprises recombinant IL18R1 peptide, or a recombinant IL18 peptide.
15. The method of any one of claims 6-7, wherein the modulator of IL18R1 activity or expression comprises a recombinant peptide comprising an amino acid sequence that is about 70%, 75%, 80%, 85%, 90%, 91%, 92%, 93%, 94%, 95%, 96%, 97%, 98%, 99%, or 100% homologous to SEQ ID NO: 8 or SEQ ID NO: 9.
16. The method of any one of claims 6-7, wherein the modulator of IL18R1 activity or expression comprises a recombinant peptide comprising an amino acid sequence that is about 70%, 75%, 80%, 85%, 90%, 91%, 92%, 93%, 94%, 95%, 96%, 97%, 98%, 99%, or 100% homologous to SEQ ID NO: 8 or SEQ ID NO: 9, and wherein the amino acid sequence is truncated at the N-terminal and/or C-terminal ends of the peptide.
17. The method of any one of claims 6-16, wherein the modulator of IL18R1 activity or expression comprises a fusion, a conjugate, or both
18. The method of any one of claims 6-17, wherein the modulator of IL18R1 activity or expression comprises an agonist or an antagonist of IL18 signaling.
19. The method of any one of claims 6-18, wherein the modulator of IL18R1 activity or expression comprises an agonist or an antagonist of IL18-IL18R1 binding.
20. The method of any one of claims 1-19, wherein the genotype is homozygous or heterozygous.
21. The method of any one or claims 1-19, wherein the disease or condition comprises and inflammatory, fibrostenotic, and/or fibrotic disease or condition.
22. The method of claim 21, wherein the inflammatory, fibrostenotic, and/or fibrotic disease or condition comprises inflammatory bowel disease (IBD), Crohn's disease (CD), perianal CD, ulcerative colitis (UC), multiple sclerosis (MS), rheumatoid arthritis (RA), primary sclerosing cholangitis (PSC), Pancolitis, primary binary cihrosis, asthma, Proctitis, Iritis, intestinal fibrosis, pulmonary fibrosis, or intestinal fibrostenosis.
23. The method of any one or claims 1-22, wherein the sample comprises whole blood, plasma, serum, or biopsy tissue.
24. The method of any one of claims 1-23, wherein subject is mammal.
25. The method of any one or claims 1-24, wherein the subject is human.
26. The method of any one of claims 1-25 wherein the subject is non-responsive to an induction of anti-Tumor Necrosis Factor (TNF) therapy, or lost response to the anti-TNF therapy after a period of time during treatment.
27. The method of any one of claims 21-26, wherein the inflammatory, fibrostenotic, and/or fibrotic disease is refractory.
28. The method of any one of claims 1-27, wherein the genotype comprises one or more single nucleotide polymorphisms (SNPs) or indels at rs1921622, rs2287037, rs1974675, rs2041739, rs76362690, rs2287037, or rs80256362, a SNP in linkage disequilibrium (LD) therewith, or any combination thereof.
29. The method of claim 28, wherein the SNP at rs1921622 comprises an “A” or a “G” on a forward DNA strand encoding the SNP.
30. The method of claim 28, wherein the SNP at rs2287037 comprises an “A” or a “G” on a reverse DNA strand encoding the SNP. In some cases, the SNP is rs10213846 and comprises a “G” or a “T” allele.
31. The method of claim 28, wherein the SNP at rs1974675 comprises a “C” or a “T” on a reverse DNA strand encoding the SNP.
32. The method of claim 28, wherein the SNP at rs2041739 comprises an “A” or a “G” on a reverse DNA strand encoding the SNP.
33. The method of claim 28, wherein the SNP at rs76362690 comprises an “A” or a “G” on a forward DNA strand encoding the SNP.
34. The method of claim 28, wherein the SNP at rs2287037 comprises an “A” or a “G” on a reverse DNA strand encoding the SNP.
35. The method of claim 28, wherein the SNP at rs80256362 comprises an “A” or a “G” on a forward DNA strand encoding the SNP.
36. The method of claim 28 or 29, wherein the Indel at rs1921622 is within SEQ ID NO: 1.
37. The method of claim 28 or 30, wherein the SNP at rs2287037 is within SEQ ID NO: 2.
38. The method of claim 28 or 31, wherein the SNP at rs1974675 is within SEQ ID NO: 3.
39. The method of claim 28 or 32, wherein the SNP at rs2041739 is within s SEQ ID NO: 4.
40. The method of claim 28 or 33, wherein the SNP at rs76362690 is within SEQ ID NO: 5.
41. The method of claim 28 or 34, wherein the SNP at rs2287037 is within SEQ ID NO: 6.
42. The method of claim 28 or 35, wherein the SNP at rs80256362 is within SEQ ID NO: 7.
43. The method of any one of claims 28-42, where LD is defined by an r² value of at least 0.80, 0.85, 0.90, 0.95, or 1.0.
44. The method of any one of claims 1-43, wherein the genotype is associated with a risk that a subject has, or will develop, inflammatory bowel disease (IBD), Crohn's disease (CD), or ulcerative colitis (UC), as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰.
45. The method of any one of claims 1-43, wherein the genotype is associated with a risk that the subject has, or will develop, a subclinical phenotype of the disease or condition as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰.
46. The method of claim 45, wherein the subclinical phenotype comprises stricturing, penetrating, or stricturing and penetrating, disease phenotypes.
47. The method of any one or claims 1-46, wherein the genotype comprises one or more SNPs in linkage disequilibrium with rs1921622 as determined by an r² value of at least about 0.80, about 0.85, about 0.90, about 0.95, or about 1.0.
48. A method of diagnosing a disease or condition in a subject, the method comprising:
   a) obtaining a sample from a subject;
   b) detecting a presence or an absence of a genotype in the sample obtained from the subject; and
   c) diagnosing the disease or condition in the subject, provided the presence of the genotype is detected in the sample obtained from the subject.
49. A method of determining whether a subject is at risk for developing a disease or condition, in a subject, the method comprising:
   a) obtaining a sample from a subject;
   b) detecting a presence or an absence of a genotype in the sample obtained from the subject; and
   c) determining the subject is at risk for developing the disease or condition, provided the presence of the genotype is detected in the sample obtained from the subject.
50. A method of determining whether a subject is suitable for treatment of a disease or condition with a modulator of Interleukin 18 Receptor 1(IL18R1) activity or expression, the method comprising:
   a) obtaining a sample from a subject;
   b) detecting a presence or an absence of a genotype in the sample obtained from the subject; and
   c) determining the subject is suitable for treatment of the disease or condition with a modulator of IL18R1, provided the presence of the genotype is detected in the sample obtained from the subject.
51. The method of any one or claims 48-50, wherein the genotype is detected with an assay comprising polymerase chain reaction (PCR), quantitative reverse-transcription PCR (qPCR), automated sequencing, genotype array, or a combination thereof.
52. The method of any one of claims 48-51, further comprising administering to the subject a modulator or IL18R1 activity or expression.
53. The method of claim 52, wherein the modulator of IL18R1 activity or expression comprises an agonist or a partial agonist of IL18R1.
54. The method of claim 52, wherein the modulator of IL18R1 activity or expression comprises an antagonist or partial antagonist of IL18R1.
55. The method of any one of claims 52-54, wherein the agonist or partial agonist comprises an antibody or antigen-binding fragment, peptide, small molecule.
56. The method of any one of claims 52-55, wherein the modulator of IL18R1 activity or expression comprises an inverse agonist.
57. The method of any one of claims 52-56, wherein the modulator of IL18R1 activity or expression comprises a positive allosteric modulator (PAM).
58. The method of any one of claims 52-56, wherein the modulator of IL18R1 activity or expression comprises a negative allosteric modulator (NAM).
59. The method of any one of claims 52-58, wherein the modulator of IL18R1 activity or expression comprises a small molecule that binds to IL18R1 or IL18, or both.
60. The method of any one of claims 52-58, wherein the modulator of IL18R1 activity or expression comprises an antibody or antigen binding fragment that binds to IL18R1 or IL18, or both.
61. The method of any one of claims 52-58, wherein the modulator of IL18R1 activity or expression comprises recombinant IL18R1 peptide, or a recombinant IL18 peptide.
62. The method of any one of claims 52-58, wherein the modulator of IL18R1 activity or expression comprises a recombinant peptide comprising an amino acid sequence that is about 70%, 75%, 80%, 85%, 90%, 91%, 92%, 93%, 94%, 95%, 96%, 97%, 98%, 99%, or 100% homologous to SEQ ID NO: 8 or SEQ ID NO: 9.
63. The method of any one of claims 52-58, wherein the modulator of IL18R1 activity or expression comprises a recombinant peptide comprising an amino acid sequence that is about 70%, 75%, 80%, 85%, 90%, 91%, 92%, 93%, 94%, 95%, 96%, 97%, 98%, 99%, or 100% homologous to SEQ ID NO: 8 or SEQ ID NO: 9, and wherein the amino acid sequence is truncated at the N-terminal and/or C-terminal ends of the peptide.
64. The method of any one of claims 52-63, wherein the modulator of IL18R1 activity or expression comprises a fusion, a conjugate, or both
65. The method of any one of claims 52-64, wherein the modulator of IL18R1 activity or expression comprises an agonist or an antagonist of IL18 signaling.
66. The method of any one of claims 52-64, wherein the modulator of IL18R1 activity or expression comprises an agonist or an antagonist of IL18-IL18R1 binding.
67. The method of any one of claims 48-66, wherein the genotype is homozygous or heterozygous.
68. The method of any one or claims 48-67, wherein the disease or condition comprises and inflammatory, fibrostenotic, and/or fibrotic disease or condition.
69. The method of claim 68, wherein the inflammatory, fibrostenotic, and/or fibrotic disease or condition comprises inflammatory bowel disease (IBD), Crohn's disease (CD), perianal CD, ulcerative colitis (UC), multiple sclerosis (MS), rheumatoid arthritis (RA), primary sclerosing cholangitis (PSC), Pancolitis, primary billary cihrosis, asthma, Proctitis, Iritis, intestinal fibrosis, pulmonary fibrosis, or intestinal fibrostenosis.
70. The method of any one or claims 48-69, wherein the sample comprises whole blood, plasma, serum, or biopsy tissue.
71. The method of any one of claims 48-70, wherein subject is mammal.
72. The method of any one or claims 48-71, wherein the subject is human.
73. The method of any one of claims 48-72, wherein the subject is non-responsive to an induction of anti-Tumor Necrosis Factor (TNF) therapy, or lost response to the anti-TNF therapy after a period of time during treatment.
74. The method of any one of claims 86-87, wherein the inflammatory, fibrostenotic, and/or fibrotic disease is refractory.
75. The method of any one of claims 48-74, wherein the genotype comprises one or more single nucleotide polymorphisms (SNPs) or indels at rs1921622, rs2287037, rs1974675, rs2041739, rs76362690, rs2287037, or rs80256362, a SNP in linkage disequilibrium (LD) therewith, or any combination thereof.
76. The method of claim 75, wherein the SNP at rs1921622 comprises an “A” or a “G” on a forward DNA strand encoding the SNP.
77. The method of claim 75, wherein the SNP at rs2287037 comprises an “A” or a “G” on a reverse DNA strand encoding the SNP. In some cases, the SNP is rs10213846 and comprises a “G” or a “T” allele.
78. The method of claim 75, wherein the SNP at rs1974675 comprises a “C” or a “T” on a reverse DNA strand encoding the SNP.
79. The method of claim 75, wherein the SNP at rs2041739 comprises an “A” or a “G” on a reverse DNA strand encoding the SNP.
80. The method of claim 75, wherein the SNP at rs76362690 comprises an “A” or a “G” on a forward DNA strand encoding the SNP.
81. The method of claim 75, wherein the SNP at rs2287037 comprises an “A” or a “G” on a reverse DNA strand encoding the SNP.
82. The method of claim 75, wherein the SNP at rs80256362 comprises an “A” or a “G” on a forward DNA strand encoding the SNP.
83. The method of claim 75 or 76, wherein the Indel at rs1921622 is within SEQ ID NO: 1.
84. The method of claim 75 or 77, wherein the SNP at rs2287037 is within SEQ ID NO: 2.
85. The method of claim 75 or 78, wherein the SNP at rs1974675 is within SEQ ID NO: 3.
86. The method of claim 75 or 79, wherein the SNP at rs2041739 is within s SEQ ID NO: 4.
87. The method of claim 75 or 80, wherein the SNP at rs76362690 is within SEQ ID NO: 5.
88. The method of claim 75 or 81, wherein the SNP at rs2287037 is within SEQ ID NO: 6.
89. The method of claim 75 or 82, wherein the SNP at rs80256362 is within SEQ ID NO: 7.
90. The method of any one of claims 75-89, where LD is defined by an r² value of at least 0.80, 0.85, 0.90, 0.95, r 1.0.
91. The method of any one of claims 48-90, wherein the genotype is associated with a risk that a subject has, or will develop, inflammatory bowel disease (IBD), Crohn's disease (CD), or ulcerative colitis (UC), as determined by a P value of at most about 1.0×10⁻⁶, about 1.0×10⁻⁷, about 1.0×10⁻⁸, about 1.0×10, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰.
92. The method of any one of claims 48-90, wherein the genotype is associated with a risk that the subject has, or will develop, a subclinical phenotype of the disease or condition as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10°, or about 1.0×10⁻¹⁰⁰.
93. The method of claim 92, wherein the subclinical phenotype comprises stricturing, penetrating, stricturing and penetrating, disease phenotypes.
94. The method of any one or claims 48-93, wherein the genotype comprises one or more SNPs in linkage disequilibrium with rs1921622 as determined by an r² value of at least about 0.80, about 0.85, about 0.90, about 0.95, or about 1.0.
95. A method for processing or analyzing a sample obtained from a subject, the method comprising:
   a) obtaining a sample from a subject;
   b) subjecting the sample to an assay by sequencing, genotype array, and/or nucleic acid amplification, to yield a data set comprising data corresponding to a presence or an absence of a genotype;
   c) in a programmed computer, inputting said data from (b) to a trained algorithm to determine whether the subject is at risk of developing, a disease or disorder, wherein the trained algorithm is trained with a plurality of training samples, and wherein said sample is independent of said plurality of training samples; and
   d) electronically outputting a report comprising the determination for the subject.
96. A method for processing or analyzing a sample obtained from a subject, the method comprising:
   a) obtaining a sample from a subject;
   b) subjecting the sample to an assay by sequencing, genotype array, and/or nucleic acid amplification, to yield a data set comprising data corresponding to a presence or an absence of a genotype;
   c) in a programmed computer, inputting said data from (b) to a trained algorithm to determine a likelihood that the subject is suitable for treatment of a disease or disorder with an agonist of IL18R1, wherein the trained algorithm is trained with a plurality of training samples, and wherein said sample is independent of said plurality of training samples; and
   d) electronically outputting a report comprising the determination for the subject.
97. The method of any one or claims 95-96, wherein (c) comprises calculating a polygenic risk score (PRS), and the PRS comprises a normalized weighted sum of a number of risk alleles within the genotype present in the subject with weights proportional to a beta value of association between the genotype with the disease or condition.
98. The method of any one of any one or claims 95-97, wherein the data set of (b) further comprises data corresponding to a presence or an absence of a surrogate genotype, provided an absence of a genotype is detected.
99. The method of claim 98, wherein the surrogate genotype is in linkage disequilibrium with the absent genotype as determined by an r² value of at least about, 0.8, about 0.85, about 0.90, about 0.95, or about 1.0.
100. The method of any one of claims 95-99, wherein the report is configured to display the determination of the subject on a user interface of an electronic device.
101. The method of claim 100, wherein the electronic device comprises a personal electronic device belonging to the subject.
102. The method of any one of claims 95-102, further comprising administering to the subject a modulator or IL18R1 activity or expression, provided the subject is determined to be at risk of having, or developing, the disease or condition.
103. The method of 102, wherein the modulator of IL18R1 activity or expression comprises an agonist or a partial agonist of IL18R1.
104. The method of claim 102, wherein the modulator of IL18R1 activity or expression comprises an antagonist or partial antagonist of IL18R1.
105. The method of any one of claims 102-104, wherein the modulator of IL18R1 activity or expression comprises an antibody or antigen-binding fragment, peptide, small molecule.
106. The method of any one of claims 102-105, wherein the modulator of IL18R1 activity or expression comprises an inverse agonist.
107. The method of any one of claims 102-105, wherein the modulator of IL18R1 activity or expression comprises a positive allosteric modulator (PAM).
108. The method of any one of claims 102-105, wherein the modulator of IL18R1 activity or expression comprises a negative allosteric modulator (NAM).
109. The method of any one of claims 102-108, wherein the modulator of IL18R1 activity or expression comprises a small molecule that binds to IL18R1 or IL18, or both.
110. The method of any one of claims 102-108, wherein the modulator of IL18R1 activity or expression comprises an antibody or antigen binding fragment that binds to IL18R1 or IL18, or both.
111. The method of any one of claims 102-108, wherein the modulator of IL18R1 activity or expression comprises recombinant IL18R1 peptide, or a recombinant IL18 peptide.
112. The method of any one of claims 102-108, wherein the modulator of IL18R1 activity or expression comprises a recombinant peptide comprising an amino acid sequence that is about 70%, 75%, 80%, 85%, 90%, 91%, 92%, 93%, 94%, 95%, 96%, 97%, 98%, 99%, or 100% homologous to SEQ ID NO: 8 or SEQ ID NO: 9.
113. The method of any one of claims 102-108, wherein the modulator of IL18R1 activity or expression comprises a recombinant peptide comprising an amino acid sequence that is about 70%, 75%, 80%, 85%, 90%, 91%, 92%, 93%, 94%, 95%, 96%, 97%, 98%, 99%, or 100% homologous to SEQ ID NO: 8 or SEQ ID NO: 9, and wherein the amino acid sequence is truncated at the N-terminal and/or C-terminal ends of the peptide.
114. The method of any one of claims 102-113, wherein the modulator of IL18R1 activity or expression comprises a fusion, a conjugate, or both
115. The method of any one of claims 102-114, wherein the modulator of IL18R1 activity or expression comprises an agonist or an antagonist of IL18 signaling.
116. The method of any one of claims 102-115, wherein the modulator of IL18R1 activity or expression comprises an agonist or an antagonist of IL18-IL18R1 binding.
117. The method of any one of claims 95-116, wherein the genotype is homozygous or heterozygous.
118. The method of any one or claims 95-117, wherein the disease or condition comprises and inflammatory, fibrostenotic, and/or fibrotic disease or condition.
119. The method of claim 118, wherein the inflammatory, fibrostenotic, and/or fibrotic disease or condition comprises inflammatory bowel disease (IBD), Crohn's disease (CD), perianal CD, ulcerative colitis (UC), multiple sclerosis (MS), rheumatoid arthritis (RA), primary sclerosing cholangitis (PSC), Pancolitis, primary billary cihrosis, asthma, Proctitis, Iritis, intestinal fibrosis, pulmonary fibrosis, or intestinal fibrostenosis.
120. The method of any one or claims 95-119, wherein the sample comprises whole blood, plasma, serum, or biopsy tissue.
121. The method of any one of claims 95-120, wherein subject is mammal.
122. The method of any one or claims 95-121, wherein the subject is human.
123. The method of any one of claims 95-122, wherein the subject is non-responsive to an induction of anti-Tumor Necrosis Factor (TNF) therapy, or lost response to the anti-TNF therapy after a period of time during treatment.
124. The method of any one of claims 118-123, wherein the inflammatory, fibrostenotic, and/or fibrotic disease is refractory.
125. The method of any one of claims 95-124, wherein the genotype comprises one or more single nucleotide polymorphisms (SNPs) or indels at rs1921622, rs2287037, rs1974675, rs2041739, rs76362690, rs2287037, or rs80256362, a SNP in linkage disequilibrium (LD) therewith, or any combination thereof.
126. The method of claim 125, wherein the SNP at rs1921622 comprises an “A” or a “G” on a forward DNA strand encoding the SNP.
127. The method of claim 125, wherein the SNP at rs2287037 comprises an “A” or a “G” on a reverse DNA strand encoding the SNP. In some cases, the SNP is rs10213846 and comprises a “G” or a “T” allele.
128. The method of claim 125, wherein the SNP at rs1974675 comprises a “C” or a “T” on a reverse DNA strand encoding the SNP.
129. The method of claim 125, wherein the SNP at rs2041739 comprises an “A” or a “G” on a reverse DNA strand encoding the SNP.
130. The method of claim 125, wherein the SNP at rs76362690 comprises an “A” or a “G” on a forward DNA strand encoding the SNP.
131. The method of claim 125, wherein the SNP at rs2287037 comprises an “A” or a “G” on a reverse DNA strand encoding the SNP.
132. The method of claim 125, wherein the SNP at rs80256362 comprises an “A” or a “G” on a forward DNA strand encoding the SNP.
133. The method of claim 125 or 126, wherein the Indel at rs1921622 is within SEQ ID NO: 1.
134. The method of claim 125 or 127, wherein the SNP at rs2287037 is within SEQ ID NO: 2.
135. The method of claim 125 or 128, wherein the SNP at rs1974675 is within SEQ ID NO: 3.
136. The method of claim 125 or 129, wherein the SNP at rs2041739 is within s SEQ ID NO: 4.
137. The method of claim 125 or 130, wherein the SNP at rs76362690 is within SEQ ID NO: 5.
138. The method of claim 125 or 131, wherein the SNP at rs2287037 is within SEQ ID NO: 6.
139. The method of claim 125 or 132, wherein the SNP at rs80256362 is within SEQ ID NO: 7.
140. The method of any one of claims 125-139, where LD is defined by an r² value of at least 0.80, 0.85, 0.90, 0.95, or 1.0.
141. The method of any one of claims 125-140, wherein the genotype is associated with a risk that a subject has, or will develop, inflammatory bowel disease (IBD), Crohn's disease (CD), or ulcerative colitis (UC), as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰.
142. The method of any one of claims 125-141, wherein the genotype is associated with a risk that the subject has, or will develop, a subclinical phenotype of the disease or condition as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰.
143. The method of claim 142 wherein the subclinical phenotype comprises stricturing, penetrating, stricturing and penetrating, disease phenotypes.
144. The method of any one or claims 125-143, wherein the genotype comprises one or more SNPs in linkage disequilibrium with rs1921622 as determined by an r² value of at least about 0.80, about 0.85, about 0.90, about 0.95, or about 1.0.
145. The method of any one of claims 125-144, wherein the genotype comprises at least about 1, 2, 3, 4, 5, 6, 7, 8, 9, 10, 11, 12, 13, or 14, single nucleotide polymorphism (SNP) or indels.
146. A method of treating a subject in need thereof with a modulator of interleukin 18 receptor 1 (IL18R1) activity or expression, wherein the subject has moderate to severe Crohn's disease (CD), and wherein the subject has a genotype characterized by the presence of one or more SNPs.
147. The method of claim 146, wherein the one or more SNPs comprises a SNP listed in Table 1.
148. The method of claim 146, wherein the single nucleotide polymorphism is associated with structuring.
149. The method of claim 148, wherein the stricturing is isolated to an ileocolonic region of an intestine.
150. The method of claim 148 or 149, wherein the one or more SNPs comprises a SNP listed in Table 2.
151. The method of claim 146, wherein the single nucleotide polymorphism is associated with a risk of a subject developing morphological defects in ileal Paneth cells.
152. The method of claim 151, wherein the one or more SNPs comprises a SNP listed in Table 3.
153. A method of treating a subject in need thereof with a modulator of interleukin 18 receptor 1 (IL18R1) activity or expression, wherein the subject has moderate to severe inflammatory bowel disease (IBD), and wherein the subject has a genotype characterized by the presence of one or more SNPs.
154. The method of claim 153, wherein the one or more SNPs comprises a SNP listed in Table 4.
155. A method of treating a subject in need thereof with a modulator of interleukin 18 receptor 1 (IL18R1) activity or expression, wherein the subject has moderate to severe ulcerative colitis, and wherein the subject has a genotype characterized by the presence of one or more SNPs.
156. The method of claim 155, wherein the one or more SNPs comprises a SNP listed in Table 5.

1. A method of treating or preventing a disease or condition in a subject, the method comprising administering a modulator of Interleukin 18 Receptor 1(IL18R1) activity or expression to the subject, provided a genotype is detected in a sample obtained the subject.

2. A method of treating or preventing a disease or condition in a subject, the method comprising:
a. obtaining a sample from a subject;
b. detecting a presence or an absence of a genotype in the sample obtained from the subject; and
c. administering to the subject a modulator of Interleukin 18 Receptor 1(IL18R1) activity or expression to the subject, provided the presence of the genotype is detected in the sample obtained from the subject.

3. The method of claim 1, wherein the modulator of IL18R1 activity or expression comprises a recombinant peptide comprising an amino acid sequence that is about 70%, 75%, 80%, 85%, 90%, 91%, 92%, 93%, 94%, 95%, 96%, 97%, 98%, 99%, or 100% homologous to SEQ ID NO: 8 or SEQ ID NO: 9.

4. The method of claim 1, wherein the modulator of IL18R1 activity or expression comprises a recombinant peptide comprising an amino acid sequence that is about 70%, 75%, 80%, 85%, 90%, 91%, 92%, 93%, 94%, 95%, 96%, 97%, 98%, 99%, or 100% homologous to SEQ ID NO: 8 or SEQ ID NO: 9, and wherein the amino acid sequence is truncated at the N-terminal and/or C-terminal ends of the peptide.

5. The method of claim 1, wherein the disease or condition comprises an inflammatory, fibrostenotic, and/or fibrotic disease or condition.

6. The method of claim 5, wherein the inflammatory, fibrostenotic, and/or fibrotic disease or condition comprises inflammatory bowel disease (IBD), Crohn's disease (CD), perianal CD, ulcerative colitis (UC), multiple sclerosis (MS), rheumatoid arthritis (RA), primary sclerosing cholangitis (PSC), Pancolitis, primary billary cihrosis, asthma, Proctitis, Iritis, intestinal fibrosis, pulmonary fibrosis, or intestinal fibrostenosis.

7. The method of claim 6, wherein the inflammatory, fibrostenotic, and/or fibrotic disease or condition comprises IBD.

8. The method of claim 1, wherein the subject is non-responsive to an induction of anti-Tumor Necrosis Factor (TNF) therapy, or lost response to the anti-TNF therapy after a period of time during treatment.

9. The method of claim 5, wherein the inflammatory, fibrostenotic, and/or fibrotic disease is refractory.

10. The method of claim 1, wherein the genotype comprises one or more single nucleotide polymorphisms (SNPs) or indels at rs76362690, rs80256362, rs1921622, rs2287037, rs1974675, or rs2041739, a SNP in linkage disequilibrium (LD) therewith, or any combination thereof.

11. The method of claim 10, wherein the genotype comprises the SNP at rs76362690, and wherein the SNP at rs76362690 is within SEQ ID NO: 5.

12. The method of claim 10, wherein the genotype comprises the SNP at rs80256362, and wherein the SNP at rs80256362 is within SEQ ID NO: 7.

13. The method of claim 10, wherein the genotype comprises the Indel at rs1921622, and wherein the Indel at rs1921622 is within SEQ ID NO: 1.

14. The method of claim 10, wherein the genotype comprises the SNP at rs1974675, and wherein the SNP at rs1974675 is within SEQ ID NO: 3.

15. The method of claim 10, wherein the genotype comprises the SNP at rs2041739, and wherein the SNP at rs2041739 is within s SEQ ID NO: 4.

16. The method of claim 10, wherein the genotype comprises the SNP at rs2287037, and wherein the SNP at rs2287037 is within SEQ ID NO: 2.

17. The method of claim 10, where the LD is defined by an r² value of at least 0.80, 0.85, 0.90, 0.95, or 1.0.

18. The method of claim 1, wherein the genotype is associated with a risk that a subject has, or will develop, inflammatory bowel disease (IBD), Crohn's disease (CD), or ulcerative colitis (UC), as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻¹⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10¹⁰⁰.

19. The method of claim 1, wherein the genotype is associated with a risk that the subject has, or will develop, a subclinical phenotype of the disease or condition as determined by a P value of at most about 1.0×10⁻⁶, about 1.0×10⁻⁷, about 1.0×10⁻⁸, about 1.0×10⁻⁹, about 1.0×10⁻¹⁰, about 1.0×10⁻²⁰, about 1.0×10⁻³⁰, about 1.0×10⁻⁴⁰, about 1.0×10⁻⁵⁰, about 1.0×10⁻⁶⁰, about 1.0×10⁻⁷⁰, about 1.0×10⁻⁸⁰, about 1.0×10⁻⁹⁰, or about 1.0×10⁻¹⁰⁰.

20. The method of claim 19, wherein the subclinical phenotype comprises stricturing, penetrating, or stricturing and penetrating, disease phenotypes.


SEQUENCE LISTING (11 sequences)

SEQ ID NO: 1 - DNA, 101 bp, Homo sapiens
  modified_base (51)..(51): a, c, t, g, unknown or other
acttcttaattctgtccataagatttgaaagaggacttaaaaattgatganttttgttctggtagccataggcactagctgaaataccttaaaagtactca

SEQ ID NO: 2 - DNA, 101 bp, Homo sapiens
  modified_base (51)..(51): a, c, t, g, unknown or other
aaaacagattcagccaaagctttcaaacaaaagtgtgcctatcttatgaangtttaaaaatcttctggcacacagatttttaaaaaaacaacctagaagat

SEQ ID NO: 3 - DNA, 103 bp, Homo sapiens
  modified_base (53)..(53): a, c, t, g, unknown or other
tctgtgtgtacatttccctctaccttcatttcttcatctctatcattgaaaantatcccttttgtatccccttccttcttagttgatttcgttcttaaaattt

SEQ ID NO: 4 - DNA, 101 bp, Homo sapiens
  modified_base (51)..(51): a, c, t, g, unknown or other
taatagaccctgaagtttcccacatcctactcctgagttcctgtgaatacnatatgttacatggcaaaaggaacctctaggtgtgattaaattaaggatct

SEQ ID NO: 5 - DNA, 101 bp, Homo sapiens
  modified_base (51)..(51): a, c, t, g, unknown or other
caaaactgtaacaaaattaagaaaaagctggttcaatgagcttagattctntgagattaatctgaaaagggagagtagttatgagaagtcttaaaaaagtg

SEQ ID NO: 6 - DNA, 101 bp, Homo sapiens; identical to SEQ ID NO: 2
  modified_base (51)..(51): a, c, t, g, unknown or other

SEQ ID NO: 7 - DNA, 101 bp, Homo sapiens
  modified_base (51)..(51): a, c, t, g, unknown or other
ctctcaaatgcctcctgaatcactgggattcctttgaggaaaaaagaaaangccttctttccccttttgcctcctctgtcctctcttcacagatgggtaat

SEQ ID NO: 8 - protein, 541 aa, Homo sapiens
MNCRELPLTLWVLISVSTAESCTSRPHITVVEGEPFYLKHCSCSLAHEIETTTKSWYKSSGSQEHVELNPRSSSRIALHDCVLEFWPVELNDTGSYFFQMKNYTQKWKLNVIRRNKHSCFTERQVTSKIVEVKKFFQITCENSYYQTLVNSTSLYKNCKKLLLENNKNPTIKKNAEFEDQGYYSCVHFLHHNGKLFNITKTFNITIVEDRSNIVPVLLGPKLNHVAVELGKNVRLNCSALLNEEDVIYWMFGEENGSDPNIHEEKEMRIMTPEGKWHASKVLRIENIGESNLNVLYNCTVASTGGTDTKSFILVRKADMADIPGHVFTRGMIIAVLILVAVVCLVTVCVIYRVDLVLFYRHLTRRDETLTDGKTYDAFVSYLKECRPENGEEHTFAVEILPRVLEKHFGYKLCIFERDVVPGGAVVDEIHSLIEKSRRLIIVLSKSYMSNEVRYELESGLHEALVERKIKIILIEFTPVTDFTFLPQSLKLLKSHRVLKWKADKSLSYNSRFWKNLLYLMPAKTVKPGRDEPEVLPVLSES

SEQ ID NO: 9 - protein, 385 aa, Homo sapiens
MNCKKLLLENNKNPTIKKNAEFEDQGYYSCVHFLHHNGKLFNITKTFNITIVEDRSNIVPVLLGPKLNHVAVELGKNVRLNCSALLNEEDVIYWMFGEENGSDPNIHEEKEMRIMTPEGKWHASKVLRIENIGESNLNVLYNCTVASTGGTDTKSFILVRKDMADIPGHVFTRGMIIAVLILVAVVCLVTVCVIYRVDLVLFYRHLTRRDETLTDGKTYDAFVSYLKECRPENGEEHTFAVEILPRVLEKHFGYKLCIFERDVVPGGAVVDEIHSLIEKSRRLIIVLSKSYMSNEVRYELESGLHEALVERKIKIILIEFTPVTDFTFLPQSLKLLKSHRVLKWKADKSLSYNSRFWKNLLYLMPAKTVKPGRDEPEVLPVLSES

SEQ ID NO: 10 - DNA, 3661 bp, Homo sapiens
tcaggaggcggagatcgctgcttctcacctactttctgaacttggcctccgcagtcgcgacctggcgtgaaggaggagctgccgcccccgccccagcctcggggacgcctctctgaagagaagccatttgaagcagaatccaaaccatgaattgtagagaattacccttgaccctttgggtgcttatatctgtaagcactgcagaatcttgtacttcacgtccccacattactgtggttgaaggggaacctttctatctgaaacattgctcgtgttcacttgcacatgagattgaaacaaccaccaaaagctggtacaaaagcagtggatcacaggaacatgtggagctgaacccaaggagttcctcgagaattgctttgcatgattgtgttttggagttttggccagttgagttgaatgacacaggatcttactttttccaaatgaaaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagcagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttaatcttcagaaacagtgaacgccaaaaagaactcaagatattctggggactgagcatatgaacctgttcataacaaaggctgtgactcgaaataattaactttgtcaaaatcctgctcacaatttgaagatgaaacttgtcattaggttggcgggaatgagactaaagattgcgctgtgggctgtggtcacgtgctcccagaagacctggaattcaaaagaaatggagctattctttttctccctctttcataactggatgcagctgctcatactcaatcccatattcagcaagtgtgaagctggacgtgatgcaaaataaccgatgccctacaaaaagggcgcatctttaagagttttaatgccagtgcttaattcgaatgaggggattttaagtgtctgaagaggcattttctagggaccagtgggtgactgagtaactgaaatgctgctttcactccctaacaccatggatctggttgtgcataggatgtgggaggaggggctggcagggccgccttcagaggctgcagggcctcagcctcaggatgcatttaatgtatcctggccacagttgcagccaacggttcttgaaagctcggtaaggccctgcaacgcagagcctgcttatgtggatctatttatgggaacttcttaaaaggaccccagaatagctctttatctttcacaagagacacaaattctaattgagttaattatctgggcctttcactttggatgctctgaaacatttgttgattttgtgtgaatgtttatatcaaaatgtttgccaggttgtattagccattgaatagcaaaaaactgatagttacttgcttgttttttaaaaattacatattaaaaatgcccttggcataaggcagcatggtgtggcagttaagagatgggctgtgcagcccatcctgagctccagtcctgagtttgctacttacttctgtggcctctggaaccttatccaacctcttggtgcttcagtttcctcatctgtgaaattagaatttataataattgcacctacctcccaggggtaactaaatgaataaatataataaagtacttacagtggttcctgacacagactcagcactccgtcagtgttgccatgactatttttattatcattattaatgattacttagatcaattatttagcagtggactaatggaagctacagagcagggaagggaagcagatctagggaggaaggcagttttgatttgaggaggtttgcacatgtagagaagcatactggagaagcatatccagagggcgaaagatatctctccattgtgcatctgcctcttttgacgttggaagacacatgtcttactccccaaagggagcccagcactgggagccttcttgatgatctcaaaaataatagctattcaagaaaatcaccaagtgactgtgaaaccgtcagttcggaaggctggttagaacatgtgggagcaacatgaatgttctacaaaagtttaaagcagagattgtttcaaatgggtgtagtagatattactgaaaaccaaaaaagagtgagattgtcagtgtaagaatgtgatttaatgtttgtagtgcttacaattttgtgtaccaactggatgactaaaaagagtaaaataatttaattaatagctcatattttatgtgtgaaaacatgttagtgaacatatataatcaaaatagatttcattgctattgcatagtctctaatacatagaatgattttgcttttctcttttattatacttgctttaaaatacttgaaatatattttgcattaaatgcatttcaagttaaatgtcttaaatgtatacattagatgtgtgttttaaaatgcataaaacacgttgaaatacattaatgaaccatt

SEQ ID NO: 11 - DNA, 3471 bp, Homo sapiens
tcaggaggcggagatcgctgcttctcacctactttctgaacttggcctccgcagtcgcgacctggcgtgaaggaggagctgccgcccccgccccagcctcggggacgcctctctgaagagaagccatttgaagcagaatccaaaccatgaattgtagagaattacccttgaccctttgggtgcttatatctgtaagcactgcagaaattatactcagaaatggaaattaaatgtcatcagaagaaataaacacagctgtttcactgaaagacaagtaactagtaaaattgtggaagttaaaaaattttttcagataacctgtgaaaacagttactatcaaacactggtcaacagcacatcattgtataagataggaccacctatttgcaggaaaacaagctcagggctccactgattctacattatgaactgtaaaaagctactactggagaacaataaaaacccaacgataaagaagaacgccgagtttgaagatcaggggtattactcctgcgtgcatttccttcatcataatggaaaactatttaatatcaccaaaaccttcaatataacaatagtggaagatcgcagtaatatagttccggttcttcttggaccaaagcttaaccatgttgcagtggaattaggaaaaaacgtaaggctcaactgctctgctttgctgaatgaagaggatgtaatttattggatgttcggggaagaaaatggatcggatcctaatatacatgaagagaaagaaatgagaattatgactccagaaggcaaatggcatgcttcaaaagtattgagaattgaaaatattggtgaaagcaatctaaatgttttatataattgcactgtggccagcacgggaggcacagacaccaaaagcttcatcttggtgagaaaagacatggctgatatcccaggccacgtcttcacaagaggaatgatcatagctgttttgatcttggtggcagtagtgtgcctagtgactgtgtgtgtcatttatagagttgacttggttctattttatagacatttaacgagaagagatgaaacattaacagatggaaaaacatatgatgcttttgtgtcttacctaaaagaatgccgacctgaaaatggagaggagcacacctttgctgtggagattttgcccagggtgttggagaaacattttgggtataagttatgcatatttgaaagggatgtagtgcctggaggagctgttgttgatgaaatccactcactgatagagaaaagccgaagactaatcattgtcctaagtaaaagttatatgtctaatgaggtcaggtatgaacttgaaagtggactccatgaagcattggtggaaagaaaaattaaaataatcttaattgaatttacacctgttactgacttcacattcttgccccaatcactaaagcttttgaaatctcacagagttctgaagtggaaggccgataaatctctttcttataactcaaggttctggaagaaccttctttacttaatgcctgcaaaaacagtcaagccaggtagagacgaaccggaagtcttgcctgttctttccgagtcttaatcttcagaaacagtgaacgccaaaaagaactcaagatattctggggactgagcatatgaacctgttcataacaaaggctgtgactcgaaataattaactttgtcaaaatcctgctcacaatttgaagatgaaacttgtcattaggttggcgggaatgagactaaagattgcgctgtgggctgtggtcacgtgctcccagaagacctggaattcaaaagaaatggagctattctttttctccctctttcataactggatgcagctgctcatactcaatcccatattcagcaagtgtgaagctggacgtgatgcaaaataaccgatgccctacaaaaagggcgcatctttaagagttttaatgccagtgcttaattcgaatgaggggattttaagtgtctgaagaggcattttctagggaccagtgggtgactgagtaactgaaatgctgctttcactccctaacaccatggatctggttgtgcataggatgtgggaggaggggctggcagggccgccttcagaggctgcagggcctcagcctcaggatgcatttaatgtatcctggccacagttgcagccaacggttcttgaaagctcggtaaggccctgcaacgcagagcctgcttatgtggatctatttatgggaacttcttaaaaggaccccagaatagctctttatctttcacaagagacacaaattctaattgagttaattatctgggcctttcactttggatgctctgaaacatttgttgattttgtgtgaatgtttatatcaaaatgtttgccaggttgtattagccattgaatagcaaaaaactgatagttacttgcttgttttttaaaaattacatattaaaaatgcccttggcataaggcagcatggtgtggcagttaagagatgggctgtgcagcccatcctgagctccagtcctgagtttgctacttacttctgtggcctctggaaccttatccaacctcttggtgcttcagtttcctcatctgtgaaattagaatttataataattgcacctacctcccaggggtaactaaatgaataaatataataaagtacttacagtggttcctgacacagactcagcactccgtcagtgttgccatgactatttttattatcattattaatgattacttagatcaattatttagcagtggactaatggaagctacagagcagggaagggaagcagatctagggaggaaggcagttttgatttgaggaggtttgcacatgtagagaagcatactggagaagcatatccagagggcgaaagatatctctccattgtgcatctgcctcttttgacgttggaagacacatgtcttactccccaaagggagcccagcactgggagccttcttgatgatctcaaaaataatagctattcaagaaaatcaccaagtgactgtgaaaccgtcagttcggaaggctggttagaacatgtgggagcaacatgaatgttctacaaaagtttaaagcagagattgtttcaaatgggtgtagtagatattactgaaaaccaaaaaagagtgagattgtcagtgtaagaatgtgatttaatgtttgtagtgcttacaattttgtgtaccaactggatgactaaaaagagtaaaataatttaattaatagctcatattttatgtgtgaaaacatgttagtgaacatatataatcaaaatagatttcattgctattgcatagtctctaatacatagaatgattttgcttttctcttttattatacttgctttaaaatacttgaaatatattttgcattaaatgcatttcaagttaaatgtcttaaatgtatacattagatgtgtgttttaaaatgcataaaacacgttgaaatacattaatgaaccatt